SEQUENCE LISTING (2302 sequences)

SEQ ID NO: 1 - DNA, 20 bp, Glycine max
  primer
ctgctgctgatgatgttcgt

SEQ ID NO: 2 - DNA, 20 bp, Glycine max
  primer
accacgaactgcgagatacc

SEQ ID NO: 3 - DNA, 20 bp, Glycine max
  primer
tttgcaactggagaacgatg

SEQ ID NO: 4 - DNA, 20 bp, Glycine max
  primer
atgagtattgggcctgacga

SEQ ID NO: 5 - DNA, 20 bp, Glycine max
  primer
tcacacactcacattccggt

SEQ ID NO: 6 - DNA, 20 bp, Glycine max
  primer
ggtccttaagtcatcagcgg

SEQ ID NO: 7 - DNA, 20 bp, Glycine max
  primer
cagcagtcagcagcagaatc

SEQ ID NO: 8 - DNA, 20 bp, Glycine max
  primer
ggaattccacaagggattga

SEQ ID NO: 9 - DNA, 20 bp, Glycine max
  primer
tcaccctcttcctcatcgtc

SEQ ID NO: 10 - DNA, 20 bp, Glycine max
  primer
ttgttgttgtctctcgctcg

SEQ ID NO: 11 - DNA, 21 bp, Glycine max
  primer
cccctatttgttttgtgagca

SEQ ID NO: 12 - DNA, 22 bp, Glycine max
  primer
cagttatgtatgggcttttcct

SEQ ID NO: 13 - DNA, 20 bp, Glycine max
  primer
gagagaaacaacagcagcga

SEQ ID NO: 14 - DNA, 20 bp, Glycine max
  primer
acttgccccacttcctcatc

SEQ ID NO: 15 - DNA, 20 bp, Glycine max
  primer
aacatcacttggcctcaacc

SEQ ID NO: 16 - DNA, 20 bp, Glycine max
  primer
gttcggactgtgagtgggat

SEQ ID NO: 17 - DNA, 20 bp, Glycine max
  primer
ccattctgattggcttctgc

SEQ ID NO: 18 - DNA, 20 bp, Glycine max
  primer
gcggaaaagagagatggatg

SEQ ID NO: 19 - DNA, 21 bp, Glycine max
  primer
tcaatctagtcgaaagccgtc

SEQ ID NO: 20 - DNA, 20 bp, Glycine max
  primer
ttccgcgtttggattactct

SEQ ID NO: 21 - DNA, 20 bp, Glycine max
  primer
cactttccacgaccacaatg

SEQ ID NO: 22 - DNA, 23 bp, Glycine max
  primer
gaagcacgagtagtgttctctct

SEQ ID NO: 23 - DNA, 20 bp, Glycine max
  primer
cgtacgcgtcaaattgagaa

SEQ ID NO: 24 - DNA, 20 bp, Glycine max
  primer
agcctttgatgtctcctcca

SEQ ID NO: 25 - DNA, 20 bp, Glycine max
  primer
cccctaggtcttccaacaca

SEQ ID NO: 26 - DNA, 20 bp, Glycine max
  primer
ctccttaggacgcaaaatgg

SEQ ID NO: 27 - DNA, 20 bp, Glycine max
  primer
ccaacaccatctcaaaatcg

SEQ ID NO: 28 - DNA, 21 bp, Glycine max
  primer
aagtgcttatttggccatgtg

SEQ ID NO: 29 - DNA, 21 bp, Glycine max
  primer
gagactcatcttcagcgacag

SEQ ID NO: 30 - DNA, 20 bp, Glycine max
  primer
ggtggggtttcagtaaccgt

SEQ ID NO: 31 - DNA, 20 bp, Glycine max
  primer
cagaggtgcattagcccttc

SEQ ID NO: 32 - DNA, 20 bp, Glycine max
  primer
catcacaattgatggatggc

SEQ ID NO: 33 - DNA, 20 bp, Glycine max
  primer
gatcaacaccaccaccacaa

SEQ ID NO: 34 - DNA, 20 bp, Glycine max
  primer
gaagggactcaccgttgcta

SEQ ID NO: 35 - DNA, 20 bp, Glycine max
  primer
aggcatcctccttcaccttt

SEQ ID NO: 36 - DNA, 20 bp, Glycine max
  primer
gaagtcctagaagcgccaag

SEQ ID NO: 37 - DNA, 21 bp, Glycine max
  primer
tctctgcctcttcttgcactc

SEQ ID NO: 38 - DNA, 20 bp, Glycine max
  primer
atgcaccaaagaacacacca

SEQ ID NO: 39 - DNA, 22 bp, Glycine max
  primer
tccagttgtattggtagcgttg

SEQ ID NO: 40 - DNA, 20 bp, Glycine max
  primer
atggtggtggtggtcgtact

SEQ ID NO: 41 - DNA, 20 bp, Glycine max
  primer
ttatgtgtatgctggagcgg

SEQ ID NO: 42 - DNA, 20 bp, Glycine max
  primer
acaacacacaaccgacctga

SEQ ID NO: 43 - DNA, 20 bp, Glycine max
  primer
tgctttccaaagaaggaagc

SEQ ID NO: 44 - DNA, 20 bp, Glycine max
  primer
ctccctctcctccttggtct

SEQ ID NO: 45 - DNA, 20 bp, Glycine max
  primer
tcaaccccttctccttcaaa

SEQ ID NO: 46 - DNA, 20 bp, Glycine max
  primer
ttttgggtggtgttgggtat

SEQ ID NO: 47 - DNA, 21 bp, Glycine max
  primer
ctgtaacatggttttgggagt

SEQ ID NO: 48 - DNA, 20 bp, Glycine max
  primer
tgctgtaacccatgatcagc

SEQ ID NO: 49 - DNA, 20 bp, Glycine max
  primer
cagcggtttcaaatgttcct

SEQ ID NO: 50 - DNA, 20 bp, Glycine max
  primer
gaggagtgagacagaggcca

SEQ ID NO: 51 - DNA, 20 bp, Glycine max
  primer
tttgggttttacgagttggc

SEQ ID NO: 52 - DNA, 20 bp, Glycine max
  primer
tggtgcctgtctcaatcaaa

SEQ ID NO: 53 - DNA, 20 bp, Glycine max
  primer
ctttgtggtgactccgttga

SEQ ID NO: 54 - DNA, 20 bp, Glycine max
  primer
ctccaactgggtcatgaggt

SEQ ID NO: 55 - DNA, 20 bp, Glycine max
  primer
ttaagccttgtcgatttccg

SEQ ID NO: 56 - DNA, 20 bp, Glycine max
  primer
gccacgaatgcgttttatct

SEQ ID NO: 57 - DNA, 20 bp, Glycine max
  primer
cacgtcagcaaacgtcagat

SEQ ID NO: 58 - DNA, 20 bp, Glycine max
  primer
ggttgtttccgacaaggaga

SEQ ID NO: 59 - DNA, 20 bp, Glycine max
  primer
ggttgtctgaaccggtcaat

SEQ ID NO: 60 - DNA, 21 bp, Glycine max
  primer
gcaacgatgaccaaactacaa

SEQ ID NO: 61 - DNA, 20 bp, Glycine max
  primer
agctctcttttgggctgaca

SEQ ID NO: 62 - DNA, 20 bp, Glycine max
  primer
cccacttcatgacccagtct

SEQ ID NO: 63 - DNA, 20 bp, Glycine max
  primer
gcagcccaaagagactcaat

SEQ ID NO: 64 - DNA, 21 bp, Glycine max
  primer
tccttccttctgcttcctttt

SEQ ID NO: 65 - DNA, 18 bp, Glycine max
  primer
catgctctcatgacttgg

SEQ ID NO: 66 - DNA, 22 bp, Glycine max
  primer
tgtgaagagacacaaagagagt

SEQ ID NO: 67 - DNA, 20 bp, Glycine max
  primer
tccagcaaaatccatcatca

SEQ ID NO: 68 - DNA, 20 bp, Glycine max
  primer
gattcattcgggaacaagga

SEQ ID NO: 69 - DNA, 20 bp, Glycine max
  primer
ttgtcgtacacaatggcagc

SEQ ID NO: 70 - DNA, 19 bp, Glycine max
  primer
gcggagataagagacccgt

SEQ ID NO: 71 - DNA, 20 bp, Glycine max
  primer
tggagtcacggcatttatga

SEQ ID NO: 72 - DNA, 20 bp, Glycine max
  primer
accctcgaagccacaaagta

SEQ ID NO: 73 - DNA, 21 bp, Glycine max
  primer
ccattccctacagttacgagc

SEQ ID NO: 74 - DNA, 20 bp, Glycine max
  primer
agcttcacctgctgcttctg

SEQ ID NO: 75 - DNA, 21 bp, Glycine max
  primer
cacgagaatggcgttttctta

SEQ ID NO: 76 - DNA, 21 bp, Glycine max
  primer
ccaaagccagagaagagacaa

SEQ ID NO: 77 - DNA, 20 bp, Glycine max
  primer
ttggacggttgaatgatttc

SEQ ID NO: 78 - DNA, 20 bp, Glycine max
  primer
cgccctaacttaatcaccct

SEQ ID NO: 79 - DNA, 20 bp, Glycine max
  primer
ggaagaagagcaggtgttgg

SEQ ID NO: 80 - DNA, 20 bp, Glycine max
  primer
atcttgggcatccaagtcag

SEQ ID NO: 81 - DNA, 23 bp, Glycine max
  primer
agtaataatatcaccaccgcacc

SEQ ID NO: 82 - DNA, 22 bp, Glycine max
  primer
tactagtctctggagaggcgtt

SEQ ID NO: 83 - DNA, 20 bp, Glycine max
  primer
tgtatctgagcaatggagcg

SEQ ID NO: 84 - DNA, 20 bp, Glycine max
  primer
aagaccaaccgagtgaaacg

SEQ ID NO: 85 - DNA, 20 bp, Glycine max
  primer
tccaatttgccagaagaacc

SEQ ID NO: 86 - DNA, 20 bp, Glycine max
  primer
cctcacacctctgtaacgcc

SEQ ID NO: 87 - DNA, 20 bp, Glycine max
  primer
aaccaaaccaaaccaaacca

SEQ ID NO: 88 - DNA, 20 bp, Glycine max
  primer
gacacagcctccatccattt

SEQ ID NO: 89 - DNA, 19 bp, Glycine max
  primer
tctcctctgtttggcgttg

SEQ ID NO: 90 - DNA, 20 bp, Glycine max
  primer
gccactttcattcccttgtg

SEQ ID NO: 91 - DNA, 20 bp, Glycine max
  primer
atccagtcgtactcgcaagc

SEQ ID NO: 92 - DNA, 22 bp, Glycine max
  primer
atgccaattttagaagagcgtc

SEQ ID NO: 93 - DNA, 19 bp, Glycine max
  primer
agctgtggaaaacccaacg

SEQ ID NO: 94 - DNA, 21 bp, Glycine max
  primer
gaataatcctttaacgccgtc

SEQ ID NO: 95 - DNA, 20 bp, Glycine max
  primer
ggagagtggatcttgggtga

SEQ ID NO: 96 - DNA, 20 bp, Glycine max
  primer
cccatttattccaccccttt

SEQ ID NO: 97 - DNA, 20 bp, Glycine max
  primer
tccatgggaagtggtaagga

SEQ ID NO: 98 - DNA, 20 bp, Glycine max
  primer
gcccgaatgtatccaatgtt

SEQ ID NO: 99 - DNA, 22 bp, Glycine max
  primer
ttgcaaagttagcagaggttga

SEQ ID NO: 100 - DNA, 21 bp, Glycine max
  primer
ttccaatatggaaccacaagc

SEQ ID NO: 101 - DNA, 20 bp, Glycine max
  primer
cgtcgccaaagtactggttt

SEQ ID NO: 102 - DNA, 20 bp, Glycine max
  primer
ttttgccaagaaattgtccc

SEQ ID NO: 103 - DNA, 20 bp, Glycine max
  primer
tgcatgaaagcaagtgacaa

SEQ ID NO: 104 - DNA, 20 bp, Glycine max
  primer
tacccctggaataaccaccc

SEQ ID NO: 105 - DNA, 20 bp, Glycine max
  primer
tttttcatctcccacttccg

SEQ ID NO: 106 - DNA, 20 bp, Glycine max
  primer
gtcaaactaaacggcgcatc

SEQ ID NO: 107 - DNA, 20 bp, Glycine max
  primer
tccatgtcatcatcctctgc

SEQ ID NO: 108 - DNA, 20 bp, Glycine max
  primer
cagctgctagtcaatccggt

SEQ ID NO: 109 - DNA, 20 bp, Glycine max
  primer
aatgcagtgtctgcaacgag

SEQ ID NO: 110 - DNA, 20 bp, Glycine max
  primer
cctccccattttcatgctta

SEQ ID NO: 111 - DNA, 20 bp, Glycine max
  primer
gaaatccgtcttccacgaaa

SEQ ID NO: 112 - DNA, 20 bp, Glycine max
  primer
tctcctcgtagcttgaaggc

SEQ ID NO: 113 - DNA, 20 bp, Glycine max
  primer
cccaaaccatttcctgagaa

SEQ ID NO: 114 - DNA, 20 bp, Glycine max
  primer
cgtgacgtccccatagaaga

SEQ ID NO: 115 - DNA, 20 bp, Glycine max
  primer
cgcttcctactcctcccttt

SEQ ID NO: 116 - DNA, 20 bp, Glycine max
  primer
ccattgttggtgcgagtttt

SEQ ID NO: 117 - DNA, 20 bp, Glycine max
  primer
gcaacaaccaagttcccttc

SEQ ID NO: 118 - DNA, 20 bp, Glycine max
  primer
agagagcgagttctgggctt

SEQ ID NO: 119 - DNA, 20 bp, Glycine max
  primer
tacaaaacctgatttgccgc

SEQ ID NO: 120 - DNA, 20 bp, Glycine max
  primer
ttcctcgcctctagacctca

SEQ ID NO: 121 - DNA, 23 bp, Glycine max
  primer
gcactactactacgcattttccg

SEQ ID NO: 122 - DNA, 20 bp, Glycine max
  primer
ggtcacaatccagacctcgt

SEQ ID NO: 123 - DNA, 20 bp, Glycine max
  primer
gagatccgtggaagaagcag

SEQ ID NO: 124 - DNA, 19 bp, Glycine max
  primer
aaattggtcttggccttgg

SEQ ID NO: 125 - DNA, 20 bp, Glycine max
  primer
acaggttttccacggatgag

SEQ ID NO: 126 - DNA, 20 bp, Glycine max
  primer
ctttgcatcaacgcagactc

SEQ ID NO: 127 - DNA, 20 bp, Glycine max
  primer
agctgaaaaggggacaacaa

SEQ ID NO: 128 - DNA, 20 bp, Glycine max
  primer
agaaggcgacgtgcataagt

SEQ ID NO: 129 - DNA, 20 bp, Glycine max
  primer
agagtcgacgctctccaaac

SEQ ID NO: 130 - DNA, 22 bp, Glycine max
  primer
gaagcttctcgagttttggact

SEQ ID NO: 131 - DNA, 20 bp, Glycine max
  primer
ctctaccttggtcagctggg

SEQ ID NO: 132 - DNA, 20 bp, Glycine max
  primer
tgggatgaccatcaagcaat

SEQ ID NO: 133 - DNA, 20 bp, Glycine max
  primer
tcgagataacggaaaccgtc

SEQ ID NO: 134 - DNA, 20 bp, Glycine max
  primer
tcgtactcggacctagtggc

SEQ ID NO: 135 - DNA, 19 bp, Glycine max
  primer
cgttggatatcgtatggcg

SEQ ID NO: 136 - DNA, 20 bp, Glycine max
  primer
aaaaccaagaaacacagcgg

SEQ ID NO: 137 - DNA, 20 bp, Glycine max
  primer
cattcgagcaactcgtttga

SEQ ID NO: 138 - DNA, 20 bp, Glycine max
  primer
aaggagcagcagaaagcaag

SEQ ID NO: 139 - DNA, 20 bp, Glycine max
  primer
gagccatagggaaacgatca

SEQ ID NO: 140 - DNA, 20 bp, Glycine max
  primer
ttgcagggaggagtttgagt

SEQ ID NO: 141 - DNA, 20 bp, Glycine max
  primer
cgcagcttctttggagtagg

SEQ ID NO: 142 - DNA, 20 bp, Glycine max
  primer
gcctcattgtgatgatggtg

SEQ ID NO: 143 - DNA, 20 bp, Glycine max
  primer
acgtcagcattggagcttct

SEQ ID NO: 144 - DNA, 20 bp, Glycine max
  primer
aatgtgcactgtggcaactc

SEQ ID NO: 145 - DNA, 20 bp, Glycine max
  primer
ttgactccccacgtggctct

SEQ ID NO: 146 - DNA, 19 bp, Glycine max
  primer
gtcgtcgccggaaagtatg

SEQ ID NO: 147 - DNA, 20 bp, Glycine max
  primer
tgggacagggattaggagtg

SEQ ID NO: 148 - DNA, 20 bp, Glycine max
  primer
ccccttttccccaataaaaa

SEQ ID NO: 149 - DNA, 20 bp, Glycine max
  primer
gacatctgggttggttgctt

SEQ ID NO: 150 - DNA, 20 bp, Glycine max
  primer
acacccttcttcggattcct

SEQ ID NO: 151 - DNA, 20 bp, Glycine max
  primer
ccatacgaagaacccaggaa

SEQ ID NO: 152 - DNA, 20 bp, Glycine max
  primer
cattttaatcccaccaacgg

SEQ ID NO: 153 - DNA, 20 bp, Glycine max
  primer
cttcctgaggatgaaaagcg

SEQ ID NO: 154 - DNA, 20 bp, Glycine max
  primer
ccgggactaagccttctctt

SEQ ID NO: 155 - DNA, 20 bp, Glycine max
  primer
aaagaggaggaagagcctgg

SEQ ID NO: 156 - DNA, 20 bp, Glycine max
  primer
agccacttcaacattccacc

SEQ ID NO: 157 - DNA, 20 bp, Glycine max
  primer
tgggaactaccaatcggaac

SEQ ID NO: 158 - DNA, 20 bp, Glycine max
  primer
aggttgatctttgaccacgg

SEQ ID NO: 159 - DNA, 20 bp, Glycine max
  primer
gctggcctttctcatacagc

SEQ ID NO: 160 - DNA, 20 bp, Glycine max
  primer
ccaaccattcattcctctgg

SEQ ID NO: 161 - DNA, 22 bp, Glycine max
  primer
acgatgtgacagaaatcagaga

SEQ ID NO: 162 - DNA, 20 bp, Glycine max
  primer
aggagcttatggcgtacgag

SEQ ID NO: 163 - DNA, 20 bp, Glycine max
  primer
attccggaaaacgtcgttag

SEQ ID NO: 164 - DNA, 20 bp, Glycine max
  primer
agagaaccgatggcacagac

SEQ ID NO: 165 - DNA, 20 bp, Glycine max
  primer
tccttccatgtctagcggag

SEQ ID NO: 166 - DNA, 20 bp, Glycine max
  primer
tgaacccagaaggaaaatga

SEQ ID NO: 167 - DNA, 20 bp, Glycine max
  primer
aggcctatgattgtgctgct

SEQ ID NO: 168 - DNA, 20 bp, Glycine max
  primer
tctccttttcctgccacaac

SEQ ID NO: 169 - DNA, 20 bp, Glycine max
  primer
ttcgtaacatgcttttcgca

SEQ ID NO: 170 - DNA, 22 bp, Glycine max
  primer
ggttgctttgccttttagtttg

SEQ ID NO: 171 - DNA, 20 bp, Glycine max
  primer
gacggagcgtgaagaagaac

SEQ ID NO: 172 - DNA, 20 bp, Glycine max
  primer
aattccacgtcagcacttcc

SEQ ID NO: 173 - DNA, 20 bp, Glycine max
  primer
ttttcttccagccagcaaat

SEQ ID NO: 174 - DNA, 20 bp, Glycine max
  primer
ctgacccactaccaccgtct

SEQ ID NO: 175 - DNA, 20 bp, Glycine max
  primer
tcatccataagggttggagc

SEQ ID NO: 176 - DNA, 20 bp, Glycine max
  primer
gtccatgtctaaggagggca

SEQ ID NO: 177 - DNA, 20 bp, Glycine max
  primer
ggaagctgctttggtctacg

SEQ ID NO: 178 - DNA, 20 bp, Glycine max
  primer
gttcaacagaggcgtgatga

SEQ ID NO: 179 - DNA, 20 bp, Glycine max
  primer
accactccctgatcagatgc

SEQ ID NO: 180 - DNA, 20 bp, Glycine max
  primer
tacccagcccatagtggttc

SEQ ID NO: 181 - DNA, 20 bp, Glycine max
  primer
cctgtctcagcacctccttc

SEQ ID NO: 182 - DNA, 20 bp, Glycine max
  primer
tcttgataagtgtgccgctg

SEQ ID NO: 183 - DNA, 20 bp, Glycine max
  primer
cgtagggagcagaagaccag

SEQ ID NO: 184 - DNA, 20 bp, Glycine max
  primer
aaaagataccgcaatggtgc

SEQ ID NO: 185 - DNA, 20 bp, Glycine max
  primer
catgggactgggagagtgtc

SEQ ID NO: 186 - DNA, 23 bp, Glycine max
  primer
tctactcctgtcaactcctgtga

SEQ ID NO: 187 - DNA, 20 bp, Glycine max
  primer
ttccctctaatgaaggcgtg

SEQ ID NO: 188 - DNA, 20 bp, Glycine max
  primer
cgcgaggaacataaacgaat

SEQ ID NO: 189 - DNA, 20 bp, Glycine max
  primer
aggcaaagggttttggagat

SEQ ID NO: 190 - DNA, 20 bp, Glycine max
  primer
ctagcggctgttagcctgtt

SEQ ID NO: 191 - DNA, 20 bp, Glycine max
  primer
cggatactctttcgtgccat

SEQ ID NO: 192 - DNA, 20 bp, Glycine max
  primer
ttgaagacgaaatcgaggct

SEQ ID NO: 193 - DNA, 20 bp, Glycine max
  primer
aaccaacaatggcacagtca

SEQ ID NO: 194 - DNA, 20 bp, Glycine max
  primer
ggatctaaaccaactccgca

SEQ ID NO: 195 - DNA, 20 bp, Glycine max
  primer
gcaaagtggttggagtggtt

SEQ ID NO: 196 - DNA, 20 bp, Glycine max
  primer
tcgaagttccccattctcac

SEQ ID NO: 197 - DNA, 20 bp, Glycine max
  primer
gtgccatctagcctgcactt

SEQ ID NO: 198 - DNA, 20 bp, Glycine max
  primer
tccatgagcatgggtctaca

SEQ ID NO: 199 - DNA, 20 bp, Glycine max
  primer
atccgtgccaccagatttag

SEQ ID NO: 200 - DNA, 20 bp, Glycine max
  primer
gtctcttctaatggctgccg

SEQ ID NO: 201 - DNA, 20 bp, Glycine max
  primer
agtattgccaccgtcagagc

SEQ ID NO: 202 - DNA, 20 bp, Glycine max
  primer
tcctcaagaagtgcagcaga

SEQ ID NO: 203 - DNA, 20 bp, Glycine max
  primer
accaagacaacctggaatgc

SEQ ID NO: 204 - DNA, 20 bp, Glycine max
  primer
atatcatcaccaagccaggg

SEQ ID NO: 205 - DNA, 20 bp, Glycine max
  primer
tcaagatggggaagttcagg

SEQ ID NO: 206 - DNA, 20 bp, Glycine max
  primer
ctggattcagtggcattcct

SEQ ID NO: 207 - DNA, 20 bp, Glycine max
  primer
tctggtgccggaatctaatc

SEQ ID NO: 208 - DNA, 20 bp, Glycine max
  primer
agtgaactcttggccttgga

SEQ ID NO: 209 - DNA, 20 bp, Glycine max
  primer
accatcctcaattttgcgtc

SEQ ID NO: 210 - DNA, 20 bp, Glycine max
  primer
tcttgtttctttgggttggc

SEQ ID NO: 211 - DNA, 20 bp, Glycine max
  primer
gggtggagaagtaggagcaa

SEQ ID NO: 212 - DNA, 20 bp, Glycine max
  primer
tgggataacaactgtggggt

SEQ ID NO: 213 - DNA, 20 bp, Glycine max
  primer
cagcaacaaccacaacaacc

SEQ ID NO: 214 - DNA, 20 bp, Glycine max
  primer
tgagctgctgaaccaaactg

SEQ ID NO: 215 - DNA, 21 bp, Glycine max
  primer
atgacatgactccacgatacg

SEQ ID NO: 216 - DNA, 23 bp, Glycine max
  primer
cacctatgctgaatctatccacg

SEQ ID NO: 217 - DNA, 20 bp, Glycine max
  primer
ccaagatccggctcctttac

SEQ ID NO: 218 - DNA, 20 bp, Glycine max
  primer
tggctgtacgtgcaaaaaga

SEQ ID NO: 219 - DNA, 20 bp, Glycine max
  primer
gtcttgcccatcttaatcgc

SEQ ID NO: 220 - DNA, 20 bp, Glycine max
  primer
taaggttgggaaattgtggc

SEQ ID NO: 221 - DNA, 20 bp, Glycine max
  primer
gcccaaccttagtgagaacg

SEQ ID NO: 222 - DNA, 20 bp, Glycine max
  primer
cgaaggtgtcttcccaacat

SEQ ID NO: 223 - DNA, 23 bp, Glycine max
  primer
gggtagggtagtaaccaaacagc

SEQ ID NO: 224 - DNA, 22 bp, Glycine max
  primer
aaaggttttcagggttgtctga

SEQ ID NO: 225 - DNA, 20 bp, Glycine max
  primer
aatttcccatggtcagcaag

SEQ ID NO: 226 - DNA, 20 bp, Glycine max
  primer
gttgcttccgactaacgtcc

SEQ ID NO: 227 - DNA, 20 bp, Glycine max
  primer
atgcttttcaagcagttggc

SEQ ID NO: 228 - DNA, 19 bp, Glycine max
  primer
aaccaaacaggcttggacc

SEQ ID NO: 229 - DNA, 20 bp, Glycine max
  primer
cgccttattcaacgcaattt

SEQ ID NO: 230 - DNA, 20 bp, Glycine max
  primer
tttgcttcagcagtgtttgg

SEQ ID NO: 231 - DNA, 20 bp, Glycine max
  primer
gaatgaggttcaggatgcgt

SEQ ID NO: 232 - DNA, 20 bp, Glycine max
  primer
cattttgatccgagccatct

SEQ ID NO: 233 - DNA, 20 bp, Glycine max
  primer
gggttccaagagatgggaat

SEQ ID NO: 234 - DNA, 20 bp, Glycine max
  primer
gcggcataacacttctctcc

SEQ ID NO: 235 - DNA, 20 bp, Glycine max
  primer
agcaatggcttcttctgcat

SEQ ID NO: 236 - DNA, 20 bp, Glycine max
  primer
ctcagaagcatgagcactgg

SEQ ID NO: 237 - DNA, 20 bp, Glycine max
  primer
gggatcggtgcactactagg

SEQ ID NO: 238 - DNA, 20 bp, Glycine max
  primer
tacaagaatgctgggccaat

SEQ ID NO: 239 - DNA, 21 bp, Glycine max
  primer
ccagctgacctatatggctgt

SEQ ID NO: 240 - DNA, 20 bp, Glycine max
  primer
tgcttttcttgtggctgcta

SEQ ID NO: 241 - DNA, 20 bp, Glycine max
  primer
cgaagagagtgctggttgtg

SEQ ID NO: 242 - DNA, 21 bp, Glycine max
  primer
cagcactaaagactgttgcga

SEQ ID NO: 243 - DNA, 20 bp, Glycine max
  primer
cgctcgcaacagtatcaaaa

SEQ ID NO: 244 - DNA, 20 bp, Glycine max
  primer
gcgccattggtagtaggaaa

SEQ ID NO: 245 - DNA, 20 bp, Glycine max
  primer
tgtccctcacttaccccatc

SEQ ID NO: 246 - DNA, 20 bp, Glycine max
  primer
tgaaactgcagggagctttt

SEQ ID NO: 247 - DNA, 20 bp, Glycine max
  primer
gttgtatccacaaccgtccc

SEQ ID NO: 248 - DNA, 20 bp, Glycine max
  primer
ggtgaggttaatgttcccca

SEQ ID NO: 249 - DNA, 20 bp, Glycine max
  primer
ggaaccagagacgtcggata

SEQ ID NO: 250 - DNA, 20 bp, Glycine max
  primer
atggtctcacagcagcattg

SEQ ID NO: 251 - DNA, 20 bp, Glycine max
  primer
ttttgaacgagtcctccacc

SEQ ID NO: 252 - DNA, 20 bp, Glycine max
  primer
aattttcccatcaaacgcct

SEQ ID NO: 253 - DNA, 20 bp, Glycine max
  primer
catgcagaatagtggtcgct

SEQ ID NO: 254 - DNA, 20 bp, Glycine max
  primer
acatgatttccgggtcaact

SEQ ID NO: 255 - DNA, 20 bp, Glycine max
  primer
cgccatgctaccaaaactaa

SEQ ID NO: 256 - DNA, 20 bp, Glycine max
  primer
tgccagctaaattaccctca

SEQ ID NO: 257 - DNA, 19 bp, Glycine max
  primer
tctctgttgtttcgcaggg

SEQ ID NO: 258 - DNA, 20 bp, Glycine max
  primer
gaagtgaactccttcgtgcc

SEQ ID NO: 259 - DNA, 20 bp, Glycine max
  primer
acgccaacaccaaccataat

SEQ ID NO: 260 - DNA, 20 bp, Glycine max
  primer
cttcttcttcgacgattccg

SEQ ID NO: 261 - DNA, 20 bp, Glycine max
  primer
atggagaggatatcgaagcg

SEQ ID NO: 262 - DNA, 20 bp, Glycine max
  primer
aacgtcactctccgtcaacc

SEQ ID NO: 263 - DNA, 20 bp, Glycine max
  primer
ttgtcgatgacaccgtagga

SEQ ID NO: 264 - DNA, 20 bp, Glycine max
  primer
cagccaaggaatcagatgct

SEQ ID NO: 265 - DNA, 20 bp, Glycine max
  primer
agaaaactggccaccacaac

SEQ ID NO: 266 - DNA, 20 bp, Glycine max
  primer
ctttggctgttccagatggt

SEQ ID NO: 267 - DNA, 20 bp, Glycine max
  primer
tcgagaatggtttccagagg

SEQ ID NO: 268 - DNA, 20 bp, Glycine max
  primer
aaagcatcacggaattttgc

SEQ ID NO: 269 - DNA, 20 bp, Glycine max
  primer
gaaccagaagaagcagtggc

SEQ ID NO: 270 - DNA, 20 bp, Glycine max
  primer
tcagacagcttgggtgtgag

SEQ ID NO: 271 - DNA, 20 bp, Glycine max
  primer
ggcttctaaggcacaggttg

SEQ ID NO: 272 - DNA, 20 bp, Glycine max
  primer
tggtttcccatccacttcat

SEQ ID NO: 273 - DNA, 20 bp, Glycine max
  primer
gtcacccaagtaacccacca

SEQ ID NO: 274 - DNA, 20 bp, Glycine max
  primer
agggcattttctcatgccta

SEQ ID NO: 275 - DNA, 20 bp, Glycine max
  primer
cgccatgacaacataaaacg

SEQ ID NO: 276 - DNA, 20 bp, Glycine max
  primer
gaagcgagaactgaaggcat

SEQ ID NO: 277 - DNA, 22 bp, Glycine max
  primer
cccgagttaatgttatggttga

SEQ ID NO: 278 - DNA, 20 bp, Glycine max
  primer
ctgtgaatgctgcgactacg

SEQ ID NO: 279 - DNA, 20 bp, Glycine max
  primer
agagaaccagtcggtgatgg

SEQ ID NO: 280 - DNA, 20 bp, Glycine max
  primer
taggcgtcaaggccatttta

SEQ ID NO: 281 - DNA, 20 bp, Glycine max
  primer
ggcattctcggaaattgatg

SEQ ID NO: 282 - DNA, 20 bp, Glycine max
  primer
caccccaccacttgactctt

SEQ ID NO: 283 - DNA, 20 bp, Glycine max
  primer
aagcttccttgggagagagg

SEQ ID NO: 284 - DNA, 20 bp, Glycine max
  primer
gctgcggaattaggagtgag

SEQ ID NO: 285 - DNA, 20 bp, Glycine max
  primer
gcagcatcaccttcctcttc

SEQ ID NO: 286 - DNA, 20 bp, Glycine max
  primer
attggcaacaagagaatcgg

SEQ ID NO: 287 - DNA, 20 bp, Glycine max
  primer
gatacccataattcgcacgc

SEQ ID NO: 288 - DNA, 21 bp, Glycine max
  primer
tcatctcctcgtgcttgtttt

SEQ ID NO: 289 - DNA, 20 bp, Glycine max
  primer
tatgctcagagggcctgttt

SEQ ID NO: 290 - DNA, 20 bp, Glycine max
  primer
acgagctttcctcccaaatc

SEQ ID NO: 291 - DNA, 20 bp, Glycine max
  primer
tgttcacctgctgaaactcg

SEQ ID NO: 292 - DNA, 20 bp, Glycine max
  primer
cgcacctagcttcattccat

SEQ ID NO: 293 - DNA, 20 bp, Glycine max
  primer
cgtcacacgtgtacctgctt

SEQ ID NO: 294 - DNA, 20 bp, Glycine max
  primer
ggtgaacggtttagcgtgtt

SEQ ID NO: 295 - DNA, 20 bp, Glycine max
  primer
ccttgcaaagctccactgtt

SEQ ID NO: 296 - DNA, 20 bp, Glycine max
  primer
ctgtgtccgctgcataagaa

SEQ ID NO: 297 - DNA, 20 bp, Glycine max
  primer
gttaaggcttggactgcctg

SEQ ID NO: 298 - DNA, 20 bp, Glycine max
  primer
gcatcaaatccacagtggtg

SEQ ID NO: 299 - DNA, 20 bp, Glycine max
  primer
gtgagcacccaaatcaacct

SEQ ID NO: 300 - DNA, 20 bp, Glycine max
  primer
ggaaacctcaggacttccct

SEQ ID NO: 301 - DNA, 20 bp, Glycine max
  primer
ttttctgatcagcgacctca

SEQ ID NO: 302 - DNA, 20 bp, Glycine max
  primer
tgacactgcctcttccttca

SEQ ID NO: 303 - DNA, 20 bp, Glycine max
  primer
tgggtgctaagctgtgtgag

SEQ ID NO: 304 - DNA, 20 bp, Glycine max
  primer
caaagctcggtctccttgag

SEQ ID NO: 305 - DNA, 20 bp, Glycine max
  primer
ctatcttcgtccatgacccc

SEQ ID NO: 306 - DNA, 20 bp, Glycine max
  primer
agttgcatgacctcccaaag

SEQ ID NO: 307 - DNA, 20 bp, Glycine max
  primer
tcccaaaactccacacatga

SEQ ID NO: 308 - DNA, 20 bp, Glycine max
  primer
tggtgagggtttgaagaagg

SEQ ID NO: 309 - DNA, 19 bp, Glycine max
  primer
ggccaagaagaacccatgt

SEQ ID NO: 310 - DNA, 20 bp, Glycine max
  primer
ggggtccaccgagttaattt

SEQ ID NO: 311 - DNA, 20 bp, Glycine max
  primer
atgggaagacaaagtcaccg

SEQ ID NO: 312 - DNA, 19 bp, Glycine max
  primer
gacttcaaattcgaggccg

SEQ ID NO: 313 - DNA, 20 bp, Glycine max
  primer
ctttgtttcctcgtttccca

SEQ ID NO: 314 - DNA, 20 bp, Glycine max
  primer
agcgctacaaagtgctggtt

SEQ ID NO: 315 - DNA, 20 bp, Glycine max
  primer
ctgagtgatgccatggagac

SEQ ID NO: 316 - DNA, 20 bp, Glycine max
  primer
ctgaacccaaccattcgttt

SEQ ID NO: 317 - DNA, 20 bp, Glycine max
  primer
accgtagacgaccacgattc

SEQ ID NO: 318 - DNA, 20 bp, Glycine max
  primer
gtggacaccgatgattttcc

SEQ ID NO: 319 - DNA, 20 bp, Glycine max
  primer
tgcatcaattatcacgcaca

SEQ ID NO: 320 - DNA, 20 bp, Glycine max
  primer
tggtgcaatacgtagccttt

SEQ ID NO: 321 - DNA, 20 bp, Glycine max
  primer
acgaccgtgattccattagc

SEQ ID NO: 322 - DNA, 21 bp, Glycine max
  primer
tgattcttttgttggacccag

SEQ ID NO: 323 - DNA, 23 bp, Glycine max
  primer
tgtacttaagctactggccaagc

SEQ ID NO: 324 - DNA, 20 bp, Glycine max
  primer
ggtgtgcacctaccatagca

SEQ ID NO: 325 - DNA, 20 bp, Glycine max
  primer
attcgttagcgtggctcatt

SEQ ID NO: 326 - DNA, 20 bp, Glycine max
  primer
gatggaccatgaattcagca

SEQ ID NO: 327 - DNA, 20 bp, Glycine max
  primer
gaaaggtcctctgcaccatc

SEQ ID NO: 328 - DNA, 20 bp, Glycine max
  primer
gtcattaaccttcttgcggc

SEQ ID NO: 329 - DNA, 20 bp, Glycine max
  primer
tgattggctctttacgagga

SEQ ID NO: 330 - DNA, 20 bp, Glycine max
  primer
tgctttgtgatttgaatggg

SEQ ID NO: 331 - DNA, 20 bp, Glycine max
  primer
tgacgtcatcgtcaaatcgt

SEQ ID NO: 332 - DNA, 20 bp, Glycine max
  primer
ttcggagacagtaaggagcg

SEQ ID NO: 333 - DNA, 20 bp, Glycine max
  primer
aaagtatcatccggtgcagg

SEQ ID NO: 334 - DNA, 20 bp, Glycine max
  primer
taattaaggtgggaaggggg

SEQ ID NO: 335 - DNA, 20 bp, Glycine max
  primer
agttggaggaaaggagagcc

SEQ ID NO: 336 - DNA, 20 bp, Glycine max
  primer
actcatgaagcccatccaag

SEQ ID NO: 337 - DNA, 20 bp, Glycine max
  primer
gcttacctcctcaacatggg

SEQ ID NO: 338 - DNA, 20 bp, Glycine max
  primer
agggaaaagatgtagccggt

SEQ ID NO: 339 - DNA, 20 bp, Glycine max
  primer
tagcatcaagattcggttcg

SEQ ID NO: 340 - DNA, 21 bp, Glycine max
  primer
tcacatgaattttaccccctg

SEQ ID NO: 341 - DNA, 20 bp, Glycine max
  primer
ccctcaaggaagcattacca

SEQ ID NO: 342 - DNA, 20 bp, Glycine max
  primer
cctgtgccatcttcaccttt

SEQ ID NO: 343 - DNA, 20 bp, Glycine max
  primer
acgatgaagacaccacctcc

SEQ ID NO: 344 - DNA, 20 bp, Glycine max
  primer
ctcaatgagcacctccttcc

SEQ ID NO: 345 - DNA, 22 bp, Glycine max
  primer
gcagattgactgctcatgatgt

SEQ ID NO: 346 - DNA, 20 bp, Glycine max
  primer
ggggctttcgttaggagttt

SEQ ID NO: 347 - DNA, 20 bp, Glycine max
  primer
cctcgcatcggagttattgt

SEQ ID NO: 348 - DNA, 20 bp, Glycine max
  primer
gagtttcaaccagcaaagcc

SEQ ID NO: 349 - DNA, 20 bp, Glycine max
  primer
ctactgccaaaggcctgaag

SEQ ID NO: 350 - DNA, 20 bp, Glycine max
  primer
ttcattgagtcgatccctcc

SEQ ID NO: 351 - DNA, 20 bp, Glycine max
  primer
aatggtggatcttccagtgc

SEQ ID NO: 352 - DNA, 20 bp, Glycine max
  primer
tggagcaattcctgataccc

SEQ ID NO: 353 - DNA, 20 bp, Glycine max
  primer
aagattccgttccttgcaga

SEQ ID NO: 354 - DNA, 20 bp, Glycine max
  primer
cactgatacgagtcctgcga

SEQ ID NO: 355 - DNA, 20 bp, Glycine max
  primer
gaacgtgctattgctgggtt

SEQ ID NO: 356 - DNA, 20 bp, Glycine max
  primer
aattgatgtggggagacgag

SEQ ID NO: 357 - DNA, 20 bp, Glycine max
  primer
tgaaggatggaatcaggagc

SEQ ID NO: 358 - DNA, 20 bp, Glycine max
  primer
cactgaagttgccacaatgc

SEQ ID NO: 359 - DNA, 20 bp, Glycine max
  primer
gccgagagacagaggagaga

SEQ ID NO: 360 - DNA, 20 bp, Glycine max
  primer
atgtacaatatggcgtcccc

SEQ ID NO: 361 - DNA, 20 bp, Glycine max
  primer
cacccagaaaacatcaatgg

SEQ ID NO: 362 - DNA, 20 bp, Glycine max
  primer
cagtgacagctccatgccta

SEQ ID NO: 363 - DNA, 20 bp, Glycine max
  primer
tgctgttgctgggtgtaatc

SEQ ID NO: 364 - DNA, 20 bp, Glycine max
  primer
aaaatgcctctcagccaatg

SEQ ID NO: 365 - DNA, 20 bp, Glycine max
  primer
accctcttggcaatcatcac

SEQ ID NO: 366 - DNA, 20 bp, Glycine max
  primer
catgtgggggtgttgttgta

SEQ ID NO: 367 - DNA, 20 bp, Glycine max
  primer
gatgaacaagggaagggctc

SEQ ID NO: 368 - DNA, 20 bp, Glycine max
  primer
acttgggatcgttaaccaaa

SEQ ID NO: 369 - DNA, 20 bp, Glycine max
  primer
ggatctaaagcttgccgtga

SEQ ID NO: 370 - DNA, 20 bp, Glycine max
  primer
gttctcacaggtctccctgg

SEQ ID NO: 371 - DNA, 22 bp, Glycine max
  primer
aaccaacaaagaacaggttagc

SEQ ID NO: 372 - DNA, 23 bp, Glycine max
  primer
tgcactaatgactcagttgaagg

SEQ ID NO: 373 - DNA, 20 bp, Glycine max
  primer
ttttgggaattttggctcag

SEQ ID NO: 374 - DNA, 20 bp, Glycine max
  primer
tcacccaccatctttcttcc

SEQ ID NO: 375 - DNA, 20 bp, Glycine max
  primer
cgagttcctcttcccacatc

SEQ ID NO: 376 - DNA, 20 bp, Glycine max
  primer
tgcaacgaagttttctccct

SEQ ID NO: 377 - DNA, 20 bp, Glycine max
  primer
tagggggcagaacatgaatc

SEQ ID NO: 378 - DNA, 20 bp, Glycine max
  primer
gttggcaggtgcagttcttt

SEQ ID NO: 379 - DNA, 20 bp, Glycine max
  primer
atccagggccatattgttga

SEQ ID NO: 380 - DNA, 20 bp, Glycine max
  primer
cttcttcgctcggaatgtgt

SEQ ID NO: 381 - DNA, 20 bp, Glycine max
  primer
accaaggttcagaagagcca

SEQ ID NO: 382 - DNA, 20 bp, Glycine max
  primer
gcaccagctgattcttcctc

SEQ ID NO: 383 - DNA, 20 bp, Glycine max
  primer
cccatcattgcatcagtgtc

SEQ ID NO: 384 - DNA, 20 bp, Glycine max
  primer
ccataagacgcatcctggtt

SEQ ID NO: 385 - DNA, 20 bp, Glycine max
  primer
gggctcctccgattttactt

SEQ ID NO: 386 - DNA, 20 bp, Glycine max
  primer
atctagtcggtgcagctggt

SEQ ID NO: 387 - DNA, 20 bp, Glycine max
  primer
catccttgtccaggaggtgt

SEQ ID NO: 388 - DNA, 20 bp, Glycine max
  primer
ccacatcaagcccttcctta

SEQ ID NO: 389 - DNA, 20 bp, Glycine max
  primer
aattcactgcctcgctcatt

SEQ ID NO: 390 - DNA, 19 bp, Glycine max
  primer
aaaggcaaaggaggcaaga

SEQ ID NO: 391 - DNA, 20 bp, Glycine max
  primer
tgaatgtgaaaccaaaccca

SEQ ID NO: 392 - DNA, 20 bp, Glycine max
  primer
ggtgaggtggaaaatggaaa

SEQ ID NO: 393 - DNA, 20 bp, Glycine max
  primer
acagcatgggaataagccct

SEQ ID NO: 394 - DNA, 20 bp, Glycine max
  primer
caagaaaagtttcgggcaaa

SEQ ID NO: 395 - DNA, 20 bp, Glycine max
  primer
ctactcgtatgccacgctca

SEQ ID NO: 396 - DNA, 20 bp, Glycine max
  primer
gccattggtgttgatggtaa

SEQ ID NO: 397 - DNA, 20 bp, Glycine max
  primer
tgatcgacgatattcccgtt

SEQ ID NO: 398 - DNA, 20 bp, Glycine max
  primer
aacaccgacattggaaggag

SEQ ID NO: 399 - DNA, 20 bp, Glycine max
  primer
gataccagtaaccggaaggc

SEQ ID NO: 400 - DNA, 20 bp, Glycine max
  primer
atgtcagtcattcaagcgca

SEQ ID NO: 401 - DNA, 20 bp, Glycine max
  primer
tgtcgtgagaaattgcgaag

SEQ ID NO: 402 - DNA, 20 bp, Glycine max
  primer
agccgcatcgcttaataatg

SEQ ID NO: 403 - DNA, 20 bp, Glycine max
  primer
ttaattcctcgcacgagctt

SEQ ID NO: 404 - DNA, 20 bp, Glycine max
  primer
tcgtttgggaaaaacaggtc

SEQ ID NO: 405 - DNA, 21 bp, Glycine max
  primer
ccaatgggactttaggtgtca

SEQ ID NO: 406 - DNA, 20 bp, Glycine max
  primer
atctagacaaggaaccccgc

SEQ ID NO: 407 - DNA, 20 bp, Glycine max
  primer
aacaggcaaaacgacgagat

SEQ ID NO: 408 - DNA, 20 bp, Glycine max
  primer
ttctgaagggtcgttggttc

SEQ ID NO: 409 - DNA, 20 bp, Glycine max
  primer
aaaacctctcttggcacgaa

SEQ ID NO: 410 - DNA, 20 bp, Glycine max
  primer
tttgagtctgcctggctctt

SEQ ID NO: 411 - DNA, 20 bp, Glycine max
  primer
caatgccaagctatgcacac

SEQ ID NO: 412 - DNA, 20 bp, Glycine max
  primer
tcccagcactcttctttgct

SEQ ID NO: 413 - DNA, 20 bp, Glycine max
  primer
attagccactgggaatgtgc

SEQ ID NO: 414 - DNA, 20 bp, Glycine max
  primer
gactcagaaggggcaaaaca

SEQ ID NO: 415 - DNA, 20 bp, Glycine max
  primer
ctcccggatagctgatgaaa

SEQ ID NO: 416 - DNA, 20 bp, Glycine max
  primer
tcaatgaatgctcaacctgc

SEQ ID NO: 417 - DNA, 20 bp, Glycine max
  primer
gattcgctccatcatcacaa

SEQ ID NO: 418 - DNA, 20 bp, Glycine max
  primer
gtgttcctcgttgacgctct

SEQ ID NO: 419 - DNA, 23 bp, Glycine max
  primer
ccactataggattccatgactga

SEQ ID NO: 420 - DNA, 22 bp, Glycine max
  primer
aatcgacagcgtacttcaactg

SEQ ID NO: 421 - DNA, 20 bp, Glycine max
  primer
gtgcaattgcctcatcttca

SEQ ID NO: 422 - DNA, 20 bp, Glycine max
  primer
ttcacggagggtacaccaat

SEQ ID NO: 423 - DNA, 20 bp, Glycine max
  primer
aacgggacagactcatgctc

SEQ ID NO: 424 - DNA, 20 bp, Glycine max
  primer
tgcacgaccagaatctgaaa

SEQ ID NO: 425 - DNA, 20 bp, Glycine max
  primer
ggaacaaccaagcaagctct

SEQ ID NO: 426 - DNA, 20 bp, Glycine max
  primer
agtccaggaacacggtcatc

SEQ ID NO: 427 - DNA, 20 bp, Glycine max
  primer
cacgtgaccgtgagctttta

SEQ ID NO: 428 - DNA, 20 bp, Glycine max
  primer
tgcccactttctcagattcc

SEQ ID NO: 429 - DNA, 20 bp, Glycine max
  primer
gactcctccccctctttcag

SEQ ID NO: 430 - DNA, 20 bp, Glycine max
  primer
ctggcctccacttcatgttt

SEQ ID NO: 431 - DNA, 20 bp, Glycine max
  primer
gctaattcctcccaatgcag

SEQ ID NO: 432 - DNA, 20 bp, Glycine max
  primer
tgctatcccaatagacgcac

SEQ ID NO: 433 - DNA, 20 bp, Glycine max
  primer
acgtgttctgcgaggacttt

SEQ ID NO: 434 - DNA, 20 bp, Glycine max
  primer
ggcttccaccagaaacaaaa

SEQ ID NO: 435 - DNA, 20 bp, Glycine max
  primer
tcagcaactacccccaagac

SEQ ID NO: 436 - DNA, 20 bp, Glycine max
  primer
ccacctggaccacctatttg

SEQ ID NO: 437 - DNA, 20 bp, Glycine max
  primer
tcagcatcaatgctctcgtc

SEQ ID NO: 438 - DNA, 20 bp, Glycine max
  primer
agcaagaaaacaagggcaga

SEQ ID NO: 439 - DNA, 20 bp, Glycine max
  primer
ggggtacggcatagtcaaac

SEQ ID NO: 440 - DNA, 20 bp, Glycine max
  primer
attttgccactcacagcctc

SEQ ID NO: 441 - DNA, 20 bp, Glycine max
  primer
atgaaaatgccctacctgcc

SEQ ID NO: 442 - DNA, 22 bp, Glycine max
  primer
tcattctaggtgtgctgagagc

SEQ ID NO: 443 - DNA, 20 bp, Glycine max
  primer
ggtgggtgtttaaggctgac

SEQ ID NO: 444 - DNA, 20 bp, Glycine max
  primer
acgcgcatatatgatcacca

SEQ ID NO: 445 - DNA, 20 bp, Glycine max
  primer
gtgttctttgtcagcagcga

SEQ ID NO: 446 - DNA, 20 bp, Glycine max
  primer
ctcatccccgacctcataga

SEQ ID NO: 447 - DNA, 20 bp, Glycine max
  primer
ttccccacacacattcttca

SEQ ID NO: 448 - DNA, 20 bp, Glycine max
  primer
tgaaccgtacacacctcgaa

SEQ ID NO: 449 - DNA, 21 bp, Glycine max
  primer
ttaaaagctggcattctgcat

SEQ ID NO: 450 - DNA, 20 bp, Glycine max
  primer
ccaaacatgaataggacccg

SEQ ID NO: 451 - DNA, 20 bp, Glycine max
  primer
ttgtgtggcagaatttccaa

SEQ ID NO: 452 - DNA, 20 bp, Glycine max
  primer
ttggttccccaaaccaaata

SEQ ID NO: 453 - DNA, 20 bp, Glycine max
  primer
tggaggagcttggaggagta

SEQ ID NO: 454 - DNA, 20 bp, Glycine max
  primer
ttccgttaacaataagcgcc

SEQ ID NO: 455 - DNA, 20 bp, Glycine max
  primer
gctccaaaaccaacaccaat

SEQ ID NO: 456 - DNA, 20 bp, Glycine max
  primer
gcaatagcttgtccacggtt

SEQ ID NO: 457 - DNA, 20 bp, Glycine max
  primer
ccgtcgtcttcctctactgg

SEQ ID NO: 458 - DNA, 20 bp, Glycine max
  primer
gggggaaatgttggagaaat

SEQ ID NO: 459 - DNA, 20 bp, Glycine max
  primer
tagaggctttggagcaggaa

SEQ ID NO: 460 - DNA, 20 bp, Glycine max
  primer
accaatagcacccaaacgag

SEQ ID NO: 461 - DNA, 20 bp, Glycine max
  primer
aggctccgacaaagacaaga

SEQ ID NO: 462 - DNA, 20 bp, Glycine max
  primer
ctctcccttgacctcacagc

SEQ ID NO: 463 - DNA, 20 bp, Glycine max
  primer
tccaacatgaaggctgaaga

SEQ ID NO: 464 - DNA, 20 bp, Glycine max
  primer
tagtacacgggcacaaatcg

SEQ ID NO: 465 - DNA, 20 bp, Glycine max
  primer
tttagaagctgggcttgacc

SEQ ID NO: 466 - DNA, 20 bp, Glycine max
  primer
aacaacgcatgacaagggat

SEQ ID NO: 467 - DNA, 20 bp, Glycine max
  primer
tctggcatgtgcactgagtt

SEQ ID NO: 468 - DNA, 20 bp, Glycine max
  primer
gtttcggtgaaacattggct

SEQ ID NO: 469 - DNA, 20 bp, Glycine max
  primer
gctattgctgggtctcaagc

SEQ ID NO: 470 - DNA, 20 bp, Glycine max
  primer
ctctccccagttctcacgac

SEQ ID NO: 471 - DNA, 20 bp, Glycine max
  primer
tatgactcggggatctttgg

SEQ ID NO: 472 - DNA, 20 bp, Glycine max
  primer
ggtagcatgcgatccaactt

SEQ ID NO: 473 - DNA, 20 bp, Glycine max
  primer
gatttctggctcacatccgt

SEQ ID NO: 474 - DNA, 20 bp, Glycine max
  primer
cagcgctcaagaaggagaag

SEQ ID NO: 475 - DNA, 20 bp, Glycine max
  primer
tgggtacagaatgagcgtga

SEQ ID NO: 476 - DNA, 20 bp, Glycine max
  primer
ttgtcgtgccagttcttcag

SEQ ID NO: 477 - DNA, 20 bp, Glycine max
  primer
tgggtacagaatgagcgtga

SEQ ID NO: 478 - DNA, 20 bp, Glycine max
  primer
tcagtttcagcctgcttcct

SEQ ID NO: 479 - DNA, 20 bp, Glycine max
  primer
ttctagctctggaccgaacc

SEQ ID NO: 480 - DNA, 20 bp, Glycine max
  primer
cctccggctctaagaaaacc

SEQ ID NO: 481 - DNA, 20 bp, Glycine max
  primer
aaccaacccgtttttcagtg

SEQ ID NO: 482 - DNA, 20 bp, Glycine max
  primer
gagaagattcacccagacgc

SEQ ID NO: 483 - DNA, 19 bp, Glycine max
  primer
tcttgccacccattggtta

SEQ ID NO: 484 - DNA, 20 bp, Glycine max
  primer
ttggacacaatctcaccgaa

SEQ ID NO: 485 - DNA, 20 bp, Glycine max
  primer
tcaagtggccaaatagtccc

SEQ ID NO: 486 - DNA, 20 bp, Glycine max
  primer
tcagcacttggaaacttgga

SEQ ID NO: 487 - DNA, 20 bp, Glycine max
  primer
gctaatggtaaggcccatga

SEQ ID NO: 488 - DNA, 20 bp, Glycine max
  primer
ttcaacaccccaaaaggaag

SEQ ID NO: 489 - DNA, 20 bp, Glycine max
  primer
gaacctgctacgccaaaaag

SEQ ID NO: 490 - DNA, 20 bp, Glycine max
  primer
tgttgttgttggtgcatgtg

SEQ ID NO: 491 - DNA, 20 bp, Glycine max
  primer
tcttctccagtgatctccga

SEQ ID NO: 492 - DNA, 20 bp, Glycine max
  primer
attgcaccaagtgtgtcctg

SEQ ID NO: 493 - DNA, 20 bp, Glycine max
  primer
agggctcatcaggtttcaga

SEQ ID NO: 494 - DNA, 20 bp, Glycine max
  primer
tgggaaacactaggaaacgg

SEQ ID NO: 495 - DNA, 20 bp, Glycine max
  primer
ccaaatcttgagcaggcttc

SEQ ID NO: 496 - DNA, 20 bp, Glycine max
  primer
aggccctccaacctgttaat

SEQ ID NO: 497 - DNA, 22 bp, Glycine max
  primer
gcacagttaatgaagttacccg

SEQ ID NO: 498 - DNA, 20 bp, Glycine max
  primer
accaggtaaaaagcccatcc

SEQ ID NO: 499 - DNA, 20 bp, Glycine max
  primer
cttgggaattgtttcctcca

SEQ ID NO: 500 - DNA, 20 bp, Glycine max
  primer
aaagatggacaggttccgtg

SEQ ID NO: 501 - DNA, 20 bp, Glycine max
  primer
cttccacaagcagtggatca

SEQ ID NO: 502 - DNA, 20 bp, Glycine max
  primer
cattgcaggttctcggagtt

SEQ ID NO: 503 - DNA, 20 bp, Glycine max
  primer
ggtatggggtgaggtacacg

SEQ ID NO: 504 - DNA, 23 bp, Glycine max
  primer
tgtatccaccgagtcatacaaca

SEQ ID NO: 505 - DNA, 20 bp, Glycine max
  primer
ttcacccaaatcaagcagaa

SEQ ID NO: 506 - DNA, 20 bp, Glycine max
  primer
tgtgagctttgtgaaccagg

SEQ ID NO: 507 - DNA, 20 bp, Glycine max
  primer
tcaatcagctcatggagtgc

SEQ ID NO: 508 - DNA, 20 bp, Glycine max
  primer
gggatgaattcactctccga

SEQ ID NO: 509 - DNA, 20 bp, Glycine max
  primer
tttcttccaggagtctgcgt

SEQ ID NO: 510 - DNA, 20 bp, Glycine max
  primer
tacagccattacacatgggg

SEQ ID NO: 511 - DNA, 20 bp, Glycine max
  primer
tggtggtggtggagacagta

SEQ ID NO: 512 - DNA, 20 bp, Glycine max
  primer
caaatcgcccaattgattct

SEQ ID NO: 513 - DNA, 22 bp, Glycine max
  primer
cctaaccaagtagcaacagcaa

SEQ ID NO: 514 - DNA, 22 bp, Glycine max
  primer
catgacaaattaggaatgaggg

SEQ ID NO: 515 - DNA, 20 bp, Glycine max
  primer
tagactgcttccgcctttgt

SEQ ID NO: 516 - DNA, 20 bp, Glycine max
  primer
agttgctggagggatgattg

SEQ ID NO: 517 - DNA, 20 bp, Glycine max
  primer
tatgagccagtcttgtcccc

SEQ ID NO: 518 - DNA, 22 bp, Glycine max
  primer
agcatcggtcatcatatcaatc

SEQ ID NO: 519 - DNA, 20 bp, Glycine max
  primer
tgtgctctgaggatcattcg

SEQ ID NO: 520 - DNA, 20 bp, Glycine max
  primer
gatgaagaagccgaagttgc

SEQ ID NO: 521 - DNA, 20 bp, Glycine max
  primer
tccagctttggaagatccac

SEQ ID NO: 522 - DNA, 20 bp, Glycine max
  primer
atccatctcactgcttccca

SEQ ID NO: 523 - DNA, 20 bp, Glycine max
  primer
ctcgagttggacctcgaaac

SEQ ID NO: 524 - DNA, 19 bp, Glycine max
  primer
agagactctttggaccgcc

SEQ ID NO: 525 - DNA, 20 bp, Glycine max
  primer
cataatgggacgtgaagtcg

SEQ ID NO: 526 - DNA, 21 bp, Glycine max
  primer
gcttgcgtagtcttgatctcc

SEQ ID NO: 527 - DNA, 20 bp, Glycine max
  primer
tggtaatgtagaggggtccg

SEQ ID NO: 528 - DNA, 21 bp, Glycine max
  primer
tcggttccagaagagttcaaa

SEQ ID NO: 529 - DNA, 20 bp, Glycine max
  primer
ttgcgtttcaacctcttcct

SEQ ID NO: 530 - DNA, 20 bp, Glycine max
  primer
gggatgggaggagatttgtt

SEQ ID NO: 531 - DNA, 20 bp, Glycine max
  primer
cgtcttgcacaaaatcgaga

SEQ ID NO: 532 - DNA, 20 bp, Glycine max
  primer
tgcacgttcaagttcttgct

SEQ ID NO: 533 - DNA, 20 bp, Glycine max
  primer
agatgcggtacatttcggag

SEQ ID NO: 534 - DNA, 20 bp, Glycine max
  primer
ggttagtgagtccagccgaa

SEQ ID NO: 535 - DNA, 20 bp, Glycine max
  primer
ctcgtttttctcgctcgact

SEQ ID NO: 536 - DNA, 20 bp, Glycine max
  primer
gatcttccatggacacgtca

SEQ ID NO: 537 - DNA, 20 bp, Glycine max
  primer
gtgggaaaggaaggatcaca

SEQ ID NO: 538 - DNA, 20 bp, Glycine max
  primer
ctgacaactgctcaagctgc

SEQ ID NO: 539 - DNA, 20 bp, Glycine max
  primer
ctccgggttctgttcacatt

SEQ ID NO: 540 - DNA, 20 bp, Glycine max
  primer
atcgcaacctatgcagctct

SEQ ID NO: 541 - DNA, 20 bp, Glycine max
  primer
gatgttttgggtgggttttg

SEQ ID NO: 542 - DNA, 20 bp, Glycine max
  primer
agcatcaacccaaactgtcc

SEQ ID NO: 543 - DNA, 20 bp, Glycine max
  primer
aggaaaagggggttggtatg

SEQ ID NO: 544 - DNA, 20 bp, Glycine max
  primer
aaaacccacccaaaacatca

SEQ ID NO: 545 - DNA, 20 bp, Glycine max
  primer
catgaatgattccaccgtga

SEQ ID NO: 546 - DNA, 21 bp, Glycine max
  primer
tcttaaccaaccaattgtggc

SEQ ID NO: 547 - DNA, 20 bp, Glycine max
  primer
catggagcaacaagcacaac

SEQ ID NO: 548 - DNA, 20 bp, Glycine max
  primer
ggaatcagtgtggctcatca

SEQ ID NO: 549 - DNA, 20 bp, Glycine max
  primer
tagggtgctgctgttccttt

SEQ ID NO: 550 - DNA, 20 bp, Glycine max
  primer
acggtcagaacttggtggag

SEQ ID NO: 551 - DNA, 20 bp, Glycine max
  primer
ttcaggactcatccccaatc

SEQ ID NO: 552 - DNA, 20 bp, Glycine max
  primer
gctgggttgcgcttatttta

SEQ ID NO: 553 - DNA, 20 bp, Glycine max
  primer
tgctggcgagaagtagaagg

SEQ ID NO: 554 - DNA, 20 bp, Glycine max
  primer
acatgctccatcattgctga

SEQ ID NO: 555 - DNA, 20 bp, Glycine max
  primer
gattgatggacgcgctaaat

SEQ ID NO: 556 - DNA, 20 bp, Glycine max
  primer
gtgatgcagagaggacagca

SEQ ID NO: 557 - DNA, 20 bp, Glycine max
  primer
cttgtcggccgctgtataat

SEQ ID NO: 558 - DNA, 20 bp, Glycine max
  primer
cccaaagtcagaatgccttg

SEQ ID NO: 559 - DNA, 20 bp, Glycine max
  primer
cgaggccaaaaactgatgat

SEQ ID NO: 560 - DNA, 20 bp, Glycine max
  primer
tttgacgcaccctctagctt

SEQ ID NO: 561 - DNA, 20 bp, Glycine max
  primer
cctgattggtcaagctccat

SEQ ID NO: 562 - DNA, 20 bp, Glycine max
  primer
aaatagggatggggagttgg

SEQ ID NO: 563 - DNA, 20 bp, Glycine max
  primer
gccactgcagacaacaacat

SEQ ID NO: 564 - DNA, 20 bp, Glycine max
  primer
attccaccgtgacgaaactc

SEQ ID NO: 565 - DNA, 20 bp, Glycine max
  primer
cttgtccccagtgcaagact

SEQ ID NO: 566 - DNA, 20 bp, Glycine max
  primer
tcagcatcgtcttcgtcatc

SEQ ID NO: 567 - DNA, 20 bp, Glycine max
  primer
cacctgagcctaagccaaag

SEQ ID NO: 568 - DNA, 20 bp, Glycine max
  primer
gcatgggcaagaattaggaa

SEQ ID NO: 569 - DNA, 20 bp, Glycine max
  primer
ttgaggactcttgcagcttg

SEQ ID NO: 570 - DNA, 20 bp, Glycine max
  primer
agtcaaagccggttgaagaa

SEQ ID NO: 571 - DNA, 20 bp, Glycine max
  primer
tcagatcctctcctcaagcc

SEQ ID NO: 572 - DNA, 20 bp, Glycine max
  primer
cccaaacgaagaaagagcaa

SEQ ID NO: 573 - DNA, 19 bp, Glycine max
  primer
cgccatgactaggggatct

SEQ ID NO: 574 - DNA, 21 bp, Glycine max
  primer
gagaaggattagtcggctgtg

SEQ ID NO: 575 - DNA, 20 bp, Glycine max
  primer
ccagcagcacaacaggagta

SEQ ID NO: 576 - DNA, 20 bp, Glycine max
  primer
ccagcactggttgcatattg

SEQ ID NO: 577 - DNA, 20 bp, Glycine max
  primer
ctctgtgccaaaggattggt

SEQ ID NO: 578 - DNA, 20 bp, Glycine max
  primer
ggagggagcacataggttga

SEQ ID NO: 579 - DNA, 20 bp, Glycine max
  primer
tcattatcggtattcggcgt

SEQ ID NO: 580 - DNA, 20 bp, Glycine max
  primer
gtctcgaatttgtgcggaat

SEQ ID NO: 581 - DNA, 20 bp, Glycine max
  primer
gttgatgtcctggagaggga

SEQ ID NO: 582 - DNA, 20 bp, Glycine max
  primer
tgtgcaaatcattggctgtt

SEQ ID NO: 583 - DNA, 20 bp, Glycine max
  primer
acacattcgggtatttccca

SEQ ID NO: 584 - DNA, 20 bp, Glycine max
  primer
agcttcaatgcatgcctctt

SEQ ID NO: 585 - DNA, 20 bp, Glycine max
  primer
caagatcactgccaaggaca

SEQ ID NO: 586 - DNA, 20 bp, Glycine max
  primer
cgccaaaatgaattgggata

SEQ ID NO: 587 - DNA, 22 bp, Glycine max
  primer
ccatgagttaacctataccggg

SEQ ID NO: 588 - DNA, 20 bp, Glycine max
  primer
ttccagcatgcagataagga

SEQ ID NO: 589 - DNA, 20 bp, Glycine max
  primer
acagcacatcatggtacgga

SEQ ID NO: 590 - DNA, 20 bp, Glycine max
  primer
catcaccaagtctgacgcat

SEQ ID NO: 591 - DNA, 20 bp, Glycine max
  primer
tctttgcccaagctatgctc

SEQ ID NO: 592 - DNA, 20 bp, Glycine max
  primer
cacaactcattcctgtgctg

SEQ ID NO: 593 - DNA, 20 bp, Glycine max
  primer
tcaagaaaccaaaactcccc

SEQ ID NO: 594 - DNA, 20 bp, Glycine max
  primer
cttcccttttcctcgacaga

SEQ ID NO: 595 - DNA, 20 bp, Glycine max
  primer
tgctcttcttcactgccctt

SEQ ID NO: 596 - DNA, 20 bp, Glycine max
  primer
tgagaatggtaggcgcttct

SEQ ID NO: 597 - DNA, 20 bp, Glycine max
  primer
atatacgatgtggcatcggg

SEQ ID NO: 598 - DNA, 20 bp, Glycine max
  primer
cgagaagctacatgcaaagc

SEQ ID NO: 599 - DNA, 20 bp, Glycine max
  primer
atactgcattccttggtcgc

SEQ ID NO: 600 - DNA, 21 bp, Glycine max
  primer
ggccatacagatctggtttca

SEQ ID NO: 601 - DNA, 20 bp, Glycine max
  primer
gccttgtggacgtcatcttt

SEQ ID NO: 602 - DNA, 20 bp, Glycine max
  primer
ggaggatgacttgcctgact

SEQ ID NO: 603 - DNA, 20 bp, Glycine max
  primer
gaaatagggtgccatgcagt

SEQ ID NO: 604 - DNA, 20 bp, Glycine max
  primer
cttttgctgccttctgttcc

SEQ ID NO: 605 - DNA, 20 bp, Glycine max
  primer
ccatgcaagaatgtgtgtcc

SEQ ID NO: 606 - DNA, 20 bp, Glycine max
  primer
agcaaatatcgtcgccattc

SEQ ID NO: 607 - DNA, 20 bp, Glycine max
  primer
aaggttggagcagtgacctg

SEQ ID NO: 608 - DNA, 20 bp, Glycine max
  primer
cttggatcttccgtccactc

SEQ ID NO: 609 - DNA, 20 bp, Glycine max
  primer
atggagggagagaagaccgt

SEQ ID NO: 610 - DNA, 20 bp, Glycine max
  primer
gcacttgatgatggtaggca

SEQ ID NO: 611 - DNA, 20 bp, Glycine max
  primer
ccgagagatggagggtgata

SEQ ID NO: 612 - DNA, 20 bp, Glycine max
  primer
gctgagcattaggacttggc

SEQ ID NO: 613 - DNA, 20 bp, Glycine max
  primer
actggcgtggaaaacatacg

SEQ ID NO: 614 - DNA, 22 bp, Glycine max
  primer
gggtacctgatccttaaattgg

SEQ ID NO: 615 - DNA, 22 bp, Glycine max
  primer
gaaacatgtatgagcatctgcc

SEQ ID NO: 616 - DNA, 20 bp, Glycine max
  primer
ccctccctctacctcacctt

SEQ ID NO: 617 - DNA, 21 bp, Glycine max
  primer
gcagcatctcttactcttccc

SEQ ID NO: 618 - DNA, 20 bp, Glycine max
  primer
aatgggcgagtacattcacg

SEQ ID NO: 619 - DNA, 20 bp, Glycine max
  primer
agtggagctaccagcctgtc

SEQ ID NO: 620 - DNA, 20 bp, Glycine max
  primer
accataaccaacttgggtgg

SEQ ID NO: 621 - DNA, 20 bp, Glycine max
  primer
aactgcacaactgaagccct

SEQ ID NO: 622 - DNA, 20 bp, Glycine max
  primer
tgcagtgatgagtttttggg

SEQ ID NO: 623 - DNA, 20 bp, Glycine max
  primer
ctgtagctgttccttcccca

SEQ ID NO: 624 - DNA, 20 bp, Glycine max
  primer
ctgctgttgttggtgttgct

SEQ ID NO: 625 - DNA, 20 bp, Glycine max
  primer
tgcaggctactttccaacct

SEQ ID NO: 626 - DNA, 20 bp, Glycine max
  primer
catacacaacccctgcaaca

SEQ ID NO: 627 - DNA, 22 bp, Glycine max
  primer
cactcttcaatttcaaacgcac

SEQ ID NO: 628 - DNA, 20 bp, Glycine max
  primer
actgagaaagcgaggtttgc

SEQ ID NO: 629 - DNA, 20 bp, Glycine max
  primer
ctaggttcaaaggccaacca

SEQ ID NO: 630 - DNA, 21 bp, Glycine max
  primer
agggaaacttgacaccatttg

SEQ ID NO: 631 - DNA, 20 bp, Glycine max
  primer
accagaatgtgcaccagtga

SEQ ID NO: 632 - DNA, 21 bp, Glycine max
  primer
tgctttgaatagggttagggg

SEQ ID NO: 633 - DNA, 22 bp, Glycine max
  primer
ctggatttctgactttgtgtgg

SEQ ID NO: 634 - DNA, 20 bp, Glycine max
  primer
tggagggtaagtccagatcg

SEQ ID NO: 635 - DNA, 20 bp, Glycine max
  primer
ccatggcccatagtaaatcg

SEQ ID NO: 636 - DNA, 20 bp, Glycine max
  primer
agacacaatgcaagaatgcg

SEQ ID NO: 637 - DNA, 20 bp, Glycine max
  primer
tgagccgagaaagaaaagga

SEQ ID NO: 638 - DNA, 23 bp, Glycine max
  primer
tcaccttaatcactctcaccgtt

SEQ ID NO: 639 - DNA, 20 bp, Glycine max
  primer
ccaaggcttgtgacctcttc

SEQ ID NO: 640 - DNA, 20 bp, Glycine max
  primer
gtgcaaagtcctccttttgc

SEQ ID NO: 641 - DNA, 20 bp, Glycine max
  primer
gctgaactgtggcttgtgaa

SEQ ID NO: 642 - DNA, 20 bp, Glycine max
  primer
ggcaacaatactcgtgcaaa

SEQ ID NO: 643 - DNA, 20 bp, Glycine max
  primer
tttagaaacacacccgctcc

SEQ ID NO: 644 - DNA, 20 bp, Glycine max
  primer
tgtcacatcaccatccacaa

SEQ ID NO: 645 - DNA, 20 bp, Glycine max
  primer
taagccaaggatgatttgcc

SEQ ID NO: 646 - DNA, 20 bp, Glycine max
  primer
actcacctttggtggtggag

SEQ ID NO: 647 - DNA, 22 bp, Glycine max
  primer
ccctagctggttttgttagctt

SEQ ID NO: 648 - DNA, 20 bp, Glycine max
  primer
caaatagctgcagcaaagca

SEQ ID NO: 649 - DNA, 20 bp, Glycine max
  primer
gaacgcatccctcaactttc

SEQ ID NO: 650 - DNA, 20 bp, Glycine max
  primer
gttgaacaagcttgcggagt

SEQ ID NO: 651 - DNA, 20 bp, Glycine max
  primer
gctgattcgtcaagtcatcg

SEQ ID NO: 652 - DNA, 20 bp, Glycine max
  primer
ggtagggttttgtggggtct

SEQ ID NO: 653 - DNA, 20 bp, Glycine max
  primer
gctgaagccctgacttgttc

SEQ ID NO: 654 - DNA, 20 bp, Glycine max
  primer
ttgacactgactggaaccca

SEQ ID NO: 655 - DNA, 20 bp, Glycine max
  primer
ggaattatggtccctgctca

SEQ ID NO: 656 - DNA, 20 bp, Glycine max
  primer
gcaaagggagcattaaacca

SEQ ID NO: 657 - DNA, 20 bp, Glycine max
  primer
tcctgatgggaaaagaccac

SEQ ID NO: 658 - DNA, 20 bp, Glycine max
  primer
cttgtcaaagctttcgaggg

SEQ ID NO: 659 - DNA, 20 bp, Glycine max
  primer
aacccttctgatcccgattc

SEQ ID NO: 660 - DNA, 20 bp, Glycine max
  primer
atttgtgttacaaaggcggg

SEQ ID NO: 661 - DNA, 20 bp, Glycine max
  primer
gctgatgctggaactgtgaa

SEQ ID NO: 662 - DNA, 20 bp, Glycine max
  primer
aacgcttgacaaggagagga

SEQ ID NO: 663 - DNA, 20 bp, Glycine max
  primer
cttccaaaagccgtgctagt

SEQ ID NO: 664 - DNA, 20 bp, Glycine max
  primer
atacgacacctcggatctgc

SEQ ID NO: 665 - DNA, 20 bp, Glycine max
  primer
aggctgatccatttggtttg

SEQ ID NO: 666 - DNA, 20 bp, Glycine max
  primer
catcgatgatccagcacttg

SEQ ID NO: 667 - DNA, 20 bp, Glycine max
  primer
ccgttcctgatctcgttgat

SEQ ID NO: 668 - DNA, 20 bp, Glycine max
  primer
gttgaagcacatccacatgc

SEQ ID NO: 669 - DNA, 20 bp, Glycine max
  primer
cgtgaaaatgcaagactcca

SEQ ID NO: 670 - DNA, 20 bp, Glycine max
  primer
cactgcattcccaacttgaa

SEQ ID NO: 671 - DNA, 20 bp, Glycine max
  primer
aggtgagtctgagccaggaa

SEQ ID NO: 672 - DNA, 20 bp, Glycine max
  primer
gaaacccagtagccatctcg

SEQ ID NO: 673 - DNA, 23 bp, Glycine max
  primer
gcttcactgtttctttgtcacac

SEQ ID NO: 674 - DNA, 19 bp, Glycine max
  primer
ccgtgcacatggaacataa

SEQ ID NO: 675 - DNA, 20 bp, Glycine max
  primer
ttctgcatcctctgatggaa

SEQ ID NO: 676 - DNA, 21 bp, Glycine max
  primer
tcaggattcaggttcattgga

SEQ ID NO: 677 - DNA, 20 bp, Glycine max
  primer
gctgcgcaggtaatcattct

SEQ ID NO: 678 - DNA, 19 bp, Glycine max
  primer
ctaggccattgcttgctca

SEQ ID NO: 679 - DNA, 20 bp, Glycine max
  primer
aaaaccgccattttgtgttt

SEQ ID NO: 680 - DNA, 21 bp, Glycine max
  primer
cgaaggagagagacagaacga

SEQ ID NO: 681 - DNA, 20 bp, Glycine max
  primer
tgagggccgttttgagatac

SEQ ID NO: 682 - DNA, 20 bp, Glycine max
  primer
agaccgacattccaccagtc

SEQ ID NO: 683 - DNA, 19 bp, Glycine max
  primer
aaagatcaattctgcgggg

SEQ ID NO: 684 - DNA, 20 bp, Glycine max
  primer
attgtcgtacaactgcgtcg

SEQ ID NO: 685 - DNA, 20 bp, Glycine max
  primer
cgcatgtcatttctgttgct

SEQ ID NO: 686 - DNA, 20 bp, Glycine max
  primer
gatggaaccagatgcagaca

SEQ ID NO: 687 - DNA, 21 bp, Glycine max
  primer
cactgatgaggtctttgtggc

SEQ ID NO: 688 - DNA, 20 bp, Glycine max
  primer
aaataaacgtggccaactgc

SEQ ID NO: 689 - DNA, 21 bp, Glycine max
  primer
aagaccatcgaaatggttgtg

SEQ ID NO: 690 - DNA, 20 bp, Glycine max
  primer
tttccctaggagcaacgcta

SEQ ID NO: 691 - DNA, 20 bp, Glycine max
  primer
tagcctcatccatttttggc

SEQ ID NO: 692 - DNA, 20 bp, Glycine max
  primer
attgcagaagggtggttgtc

SEQ ID NO: 693 - DNA, 19 bp, Glycine max
  primer
ggatctcgcgaaaccgtta

SEQ ID NO: 694 - DNA, 20 bp, Glycine max
  primer
agcctaagcctctccacctc

SEQ ID NO: 695 - DNA, 20 bp, Glycine max
  primer
gttgctgctgcctatgactg

SEQ ID NO: 696 - DNA, 20 bp, Glycine max
  primer
aaccgttgtgtccggattag

SEQ ID NO: 697 - DNA, 20 bp, Glycine max
  primer
ctgaggaggtggctcagaac

SEQ ID NO: 698 - DNA, 20 bp, Glycine max
  primer
gcaggtgatgttgtgcagtt

SEQ ID NO: 699 - DNA, 20 bp, Glycine max
  primer
aatgacattttgctctgggc

SEQ ID NO: 700 - DNA, 20 bp, Glycine max
  primer
agtacgtttgtcctcgctgc

SEQ ID NO: 701 - DNA, 20 bp, Glycine max
  primer
taaagccaatcatgacaccg

SEQ ID NO: 702 - DNA, 20 bp, Glycine max
  primer
tttcagggaaaggagctgaa

SEQ ID NO: 703 - DNA, 20 bp, Glycine max
  primer
acttttgttatggccaaccg

SEQ ID NO: 704 - DNA, 20 bp, Glycine max
  primer
cgtcaccgtactctcgttca

SEQ ID NO: 705 - DNA, 20 bp, Glycine max
  primer
agaaaggcccgttggactat

SEQ ID NO: 706 - DNA, 20 bp, Glycine max
  primer
aagtagccaaacggcaaaga

SEQ ID NO: 707 - DNA, 20 bp, Glycine max
  primer
tgtcttctcttccaccaccc

SEQ ID NO: 708 - DNA, 20 bp, Glycine max
  primer
ccatcctgccgaagtaagaa

SEQ ID NO: 709 - DNA, 20 bp, Glycine max
  primer
gccgatccaaatcgtcttta

SEQ ID NO: 710 - DNA, 20 bp, Glycine max
  primer
gcaaaagggattctcaaagc

SEQ ID NO: 711 - DNA, 20 bp, Glycine max
  primer
gttggctacaatgccactcc

SEQ ID NO: 712 - DNA, 19 bp, Glycine max
  primer
aagccacgtcctggaaatc

SEQ ID NO: 713 - DNA, 20 bp, Glycine max
  primer
aatggctgcaaaataccgag

SEQ ID NO: 714 - DNA, 20 bp, Glycine max
  primer
actcagaccccaaatgcaaa

SEQ ID NO: 715 - DNA, 20 bp, Glycine max
  primer
atttcaacatccttcagccg

SEQ ID NO: 716 - DNA, 19 bp, Glycine max
  primer
agtgcaaagtggggtgatt

SEQ ID NO: 717 - DNA, 20 bp, Glycine max
  primer
cttttcccccaaatttcgtt

SEQ ID NO: 718 - DNA, 20 bp, Glycine max
  primer
aatcatgaacccctgcaaag

SEQ ID NO: 719 - DNA, 20 bp, Glycine max
  primer
gcaactcttccaaggcattc

SEQ ID NO: 720 - DNA, 20 bp, Glycine max
  primer
tcctctgcctatggacaagc

SEQ ID NO: 721 - DNA, 20 bp, Glycine max
  primer
taaaagaagacacggcaccc

SEQ ID NO: 722 - DNA, 20 bp, Glycine max
  primer
ggagtttgtgcaatgtgtgg

SEQ ID NO: 723 - DNA, 20 bp, Glycine max
  primer
gccctacaatcgaagggaat

SEQ ID NO: 724 - DNA, 21 bp, Glycine max
  primer
tgatggccttgtagcctaatg

SEQ ID NO: 725 - DNA, 20 bp, Glycine max
  primer
caatatctgccagggcttgt

SEQ ID NO: 726 - DNA, 20 bp, Glycine max
  primer
aagagtgcctttgaggcaga

SEQ ID NO: 727 - DNA, 20 bp, Glycine max
  primer
tcaagatttgttcggccagt

SEQ ID NO: 728 - DNA, 20 bp, Glycine max
  primer
ccgccatcaggacatctaat

SEQ ID NO: 729 - DNA, 20 bp, Glycine max
  primer
ctctccctccagatgtcagc

SEQ ID NO: 730 - DNA, 20 bp, Glycine max
  primer
tggcttaaccttcgttccac

SEQ ID NO: 731 - DNA, 20 bp, Glycine max
  primer
tccaaacatccttttccgtg

SEQ ID NO: 732 - DNA, 20 bp, Glycine max
  primer
gtgtgaggggaaaaacatgg

SEQ ID NO: 733 - DNA, 20 bp, Glycine max
  primer
tttggtcaaacatgcagagg

SEQ ID NO: 734 - DNA, 20 bp, Glycine max
  primer
gagaccaatgccttccaaaa

SEQ ID NO: 735 - DNA, 20 bp, Glycine max
  primer
ttcgatcgaggaactgagtg

SEQ ID NO: 736 - DNA, 20 bp, Glycine max
  primer
agatggttcagcaaagcagc

SEQ ID NO: 737 - DNA, 20 bp, Glycine max
  primer
tatcacttccaaacgccctt

SEQ ID NO: 738 - DNA, 20 bp, Glycine max
  primer
ttctgaagggaagacatggg

SEQ ID NO: 739 - DNA, 20 bp, Glycine max
  primer
cgggcttctatcgtgtcatt

SEQ ID NO: 740 - DNA, 20 bp, Glycine max
  primer
ctgattacatgggagcacga

SEQ ID NO: 741 - DNA, 20 bp, Glycine max
  primer
gaggccacagaagacagtcc

SEQ ID NO: 742 - DNA, 20 bp, Glycine max
  primer
gatcctgccgaatgaagtgt

SEQ ID NO: 743 - DNA, 20 bp, Glycine max
  primer
aagactgccagttcacagcc

SEQ ID NO: 744 - DNA, 23 bp, Glycine max
  primer
caagagatcttcttctgcgaatg

SEQ ID NO: 745 - DNA, 20 bp, Glycine max
  primer
gaagcacaaatgggtggagt

SEQ ID NO: 746 - DNA, 20 bp, Glycine max
  primer
tcaggtgctggtagttgtgc

SEQ ID NO: 747 - DNA, 20 bp, Glycine max
  primer
tattggagcttgagccgcta

SEQ ID NO: 748 - DNA, 20 bp, Glycine max
  primer
tccatccgagacaatgatga

SEQ ID NO: 749 - DNA, 19 bp, Glycine max
  primer
accttctcagcagcttcgc

SEQ ID NO: 750 - DNA, 20 bp, Glycine max
  primer
gctccctgcaaattgtcatt

SEQ ID NO: 751 - DNA, 20 bp, Glycine max
  primer
aatgcaaaagagtccttcgg

SEQ ID NO: 752 - DNA, 23 bp, Glycine max
  primer
gcttgactttgttgtaccattcc

SEQ ID NO: 753 - DNA, 20 bp, Glycine max
  primer
accacttcctcaggacaacg

SEQ ID NO: 754 - DNA, 20 bp, Glycine max
  primer
tacacttacaccccacccgt

SEQ ID NO: 755 - DNA, 20 bp, Glycine max
  primer
tgggctaagatcccttcctt

SEQ ID NO: 756 - DNA, 20 bp, Glycine max
  primer
atccaaaggagcagaaagca

SEQ ID NO: 757 - DNA, 20 bp, Glycine max
  primer
aggtgtcctttgccttgtca

SEQ ID NO: 758 - DNA, 20 bp, Glycine max
  primer
cagcagccaagattgtttca

SEQ ID NO: 759 - DNA, 20 bp, Glycine max
  primer
cggagttgatcactgggatt

SEQ ID NO: 760 - DNA, 20 bp, Glycine max
  primer
tccagaaaacaagccgagat

SEQ ID NO: 761 - DNA, 20 bp, Glycine max
  primer
gctctggacaatggacatca

SEQ ID NO: 762 - DNA, 20 bp, Glycine max
  primer
taaacaaatcccgaatgcac

SEQ ID NO: 763 - DNA, 20 bp, Glycine max
  primer
ccgaaatcggtttgacgtat

SEQ ID NO: 764 - DNA, 20 bp, Glycine max
  primer
gaacgtgacaaaggggaaga

SEQ ID NO: 765 - DNA, 20 bp, Glycine max
  primer
gatggttgtgatggggaaac

SEQ ID NO: 766 - DNA, 20 bp, Glycine max
  primer
ttatgcaatgagcaatccca

SEQ ID NO: 767 - DNA, 20 bp, Glycine max
  primer
agggcttaagcttttcgcac

SEQ ID NO: 768 - DNA, 21 bp, Glycine max
  primer
ttgcgtggatcatatcctttc

SEQ ID NO: 769 - DNA, 20 bp, Glycine max
  primer
gacttgctggtggtggaaat

SEQ ID NO: 770 - DNA, 20 bp, Glycine max
  primer
tcatcatttctctgggaggg

SEQ ID NO: 771 - DNA, 20 bp, Glycine max
  primer
gttttgccacgtgaaatcct

SEQ ID NO: 772 - DNA, 20 bp, Glycine max
  primer
cggtgcagttaagccagttt

SEQ ID NO: 773 - DNA, 20 bp, Glycine max
  primer
gctgcagcatgaaaatcaaa

SEQ ID NO: 774 - DNA, 20 bp, Glycine max
  primer
ggcggactacacatagtggg

SEQ ID NO: 775 - DNA, 20 bp, Glycine max
  primer
aggctgcattcttggctaaa

SEQ ID NO: 776 - DNA, 20 bp, Glycine max
  primer
attatgcctttccccattcc

SEQ ID NO: 777 - DNA, 20 bp, Glycine max
  primer
tacccttaccaaccccatca

SEQ ID NO: 778 - DNA, 20 bp, Glycine max
  primer
gtgggggagaaggagtagga

SEQ ID NO: 779 - DNA, 20 bp, Glycine max
  primer
gcttcttgtcatctctgggg

SEQ ID NO: 780 - DNA, 20 bp, Glycine max
  primer
acgtccccattctttcacag

SEQ ID NO: 781 - DNA, 20 bp, Glycine max
  primer
cgttcacgtgattgatttcg

SEQ ID NO: 782 - DNA, 19 bp, Glycine max
  primer
agtcggaaaaccggaggac

SEQ ID NO: 783 - DNA, 20 bp, Glycine max
  primer
ccgagtcgcggttaaagtag

SEQ ID NO: 784 - DNA, 20 bp, Glycine max
  primer
taacacaagcagatgcgacg

SEQ ID NO: 785 - DNA, 20 bp, Glycine max
  primer
tccacatttgaaaatcaccg

SEQ ID NO: 786 - DNA, 20 bp, Glycine max
  primer
ccaacttttctgcctcctca

SEQ ID NO: 787 - DNA, 20 bp, Glycine max
  primer
tcatcaaatctgacggttgc

SEQ ID NO: 788 - DNA, 20 bp, Glycine max
  primer
tggtcgaagagaatggttcc

SEQ ID NO: 789 - DNA, 20 bp, Glycine max
  primer
cttcccttcgagttcttccc

SEQ ID NO: 790 - DNA, 20 bp, Glycine max
  primer
gattgcctcgttaggtcgaa

SEQ ID NO: 791 - DNA, 20 bp, Glycine max
  primer
aatgctcctttctttgccac

SEQ ID NO: 792 - DNA, 20 bp, Glycine max
  primer
aacctccattcgttttcacg

SEQ ID NO: 793 - DNA, 20 bp, Glycine max
  primer
attcctggcatagcagccta

SEQ ID NO: 794 - DNA, 20 bp, Glycine max
  primer
ggcgcttgttgatgttgtta

SEQ ID NO: 795 - DNA, 20 bp, Glycine max
  primer
tcccaaggtacaactcggac

SEQ ID NO: 796 - DNA, 20 bp, Glycine max
  primer
tccagtcttttcgactcgct

SEQ ID NO: 797 - DNA, 20 bp, Glycine max
  primer
gcaggcatcagagcaacata

SEQ ID NO: 798 - DNA, 20 bp, Glycine max
  primer
atttcgactccgatactgcg

SEQ ID NO: 799 - DNA, 20 bp, Glycine max
  primer
ttctcaaagaattgcggctt

SEQ ID NO: 800 - DNA, 20 bp, Glycine max
  primer
ggaggttccttgcatctcaa

SEQ ID NO: 801 - DNA, 20 bp, Glycine max
  primer
agccaaagctccacatcatc

SEQ ID NO: 802 - DNA, 20 bp, Glycine max
  primer
tgaggtgtctcatcgtttcg

SEQ ID NO: 803 - DNA, 20 bp, Glycine max
  primer
tctcttagccaccaattccg

SEQ ID NO: 804 - DNA, 20 bp, Glycine max
  primer
aagattgatgtgtggagggc

SEQ ID NO: 805 - DNA, 20 bp, Glycine max
  primer
gcgtggtggattttgagatt

SEQ ID NO: 806 - DNA, 20 bp, Glycine max
  primer
tcctttttctgctacggctg

SEQ ID NO: 807 - DNA, 20 bp, Glycine max
  primer
tggctctggctcaattctct

SEQ ID NO: 808 - DNA, 20 bp, Glycine max
  primer
gggaattggaggaggatgat

SEQ ID NO: 809 - DNA, 20 bp, Glycine max
  primer
tttatcctcttgctgcctcg

SEQ ID NO: 810 - DNA, 21 bp, Glycine max
  primer
ggttgaacttgttcgagtgga

SEQ ID NO: 811 - DNA, 20 bp, Glycine max
  primer
aaaaaccccaaccaaagtca

SEQ ID NO: 812 - DNA, 20 bp, Glycine max
  primer
acacgggaagagtggtgaat

SEQ ID NO: 813 - DNA, 20 bp, Glycine max
  primer
tttgtgagggcatctgtgag

SEQ ID NO: 814 - DNA, 20 bp, Glycine max
  primer
catcttggggctcagaacat

SEQ ID NO: 815 - DNA, 20 bp, Glycine max
  primer
cttctgggggatggattttt

SEQ ID NO: 816 - DNA, 20 bp, Glycine max
  primer
gccctttcagtgacatctcc

SEQ ID NO: 817 - DNA, 20 bp, Glycine max
  primer
ccattttccattggttggac

SEQ ID NO: 818 - DNA, 20 bp, Glycine max
  primer
gccaatcctatttgggatga

SEQ ID NO: 819 - DNA, 20 bp, Glycine max
  primer
ctcgcctcaaggagtcaaag

SEQ ID NO: 820 - DNA, 20 bp, Glycine max
  primer
aaagattacgtggcgaggtg

SEQ ID NO: 821 - DNA, 20 bp, Glycine max
  primer
ctaatacggtgacggtggct

SEQ ID NO: 822 - DNA, 20 bp, Glycine max
  primer
ccagcaatcggagatgagtt

SEQ ID NO: 823 - DNA, 20 bp, Glycine max
  primer
aaatgaggctgcaaaagcat

SEQ ID NO: 824 - DNA, 20 bp, Glycine max
  primer
gatgcaatggcagaaggaat

SEQ ID NO: 825 - DNA, 19 bp, Glycine max
  primer
aacccaacacgactccaca

SEQ ID NO: 826 - DNA, 20 bp, Glycine max
  primer
gcacgaggctaggaagagag

SEQ ID NO: 827 - DNA, 22 bp, Glycine max
  primer
tctcttggtcatcatggaacat

SEQ ID NO: 828 - DNA, 20 bp, Glycine max
  primer
tttacgaagtcccttgcacc

SEQ ID NO: 829 - DNA, 20 bp, Glycine max
  primer
aaataattggcgtttggctg

SEQ ID NO: 830 - DNA, 20 bp, Glycine max
  primer
atcccatcagaagcaactgg

SEQ ID NO: 831 - DNA, 20 bp, Glycine max
  primer
ctgcgtttacacggatgaaa

SEQ ID NO: 832 - DNA, 20 bp, Glycine max
  primer
ctggctcctcctaagtgcat

SEQ ID NO: 833 - DNA, 20 bp, Glycine max
  primer
gcggtgcagtctgattacaa

SEQ ID NO: 834 - DNA, 20 bp, Glycine max
  primer
tctccacccttgagaaaacg

SEQ ID NO: 835 - DNA, 20 bp, Glycine max
  primer
caactaccgagcaaacccat

SEQ ID NO: 836 - DNA, 20 bp, Glycine max
  primer
catgcccaactcaaagtgtg

SEQ ID NO: 837 - DNA, 20 bp, Glycine max
  primer
tggtgttccagacgatgaag

SEQ ID NO: 838 - DNA, 20 bp, Glycine max
  primer
tctcaccaaacccttccaac

SEQ ID NO: 839 - DNA, 22 bp, Glycine max
  primer
cattgaactagctgggtgacag

SEQ ID NO: 840 - DNA, 20 bp, Glycine max
  primer
ttgggccaagaaattgagtc

SEQ ID NO: 841 - DNA, 20 bp, Glycine max
  primer
attccgcttcattgtatggc

SEQ ID NO: 842 - DNA, 20 bp, Glycine max
  primer
aagttgacggacgaaactgg

SEQ ID NO: 843 - DNA, 20 bp, Glycine max
  primer
gattggccaacacattgaca

SEQ ID NO: 844 - DNA, 20 bp, Glycine max
  primer
gtgagggttttgagggtgaa

SEQ ID NO: 845 - DNA, 20 bp, Glycine max
  primer
ttggcttaggaagtttggga

SEQ ID NO: 846 - DNA, 20 bp, Glycine max
  primer
ggttgaccagcttgaccatt

SEQ ID NO: 847 - DNA, 19 bp, Glycine max
  primer
gaagcttgtgttcgtgcgt

SEQ ID NO: 848 - DNA, 21 bp, Glycine max
  primer
gcggacatatggataggaaaa

SEQ ID NO: 849 - DNA, 20 bp, Glycine max
  primer
gaagcagtgacatgtggtgg

SEQ ID NO: 850 - DNA, 20 bp, Glycine max
  primer
atcttgctcagaaacggagg

SEQ ID NO: 851 - DNA, 20 bp, Glycine max
  primer
tcaaagggtgtgcaactgac

SEQ ID NO: 852 - DNA, 20 bp, Glycine max
  primer
tttcggattccctacagcac

SEQ ID NO: 853 - DNA, 21 bp, Glycine max
  primer
tcactatagggaatttggccc

SEQ ID NO: 854 - DNA, 21 bp, Glycine max
  primer
ttcaacactaccctcaatggc

SEQ ID NO: 855 - DNA, 20 bp, Glycine max
  primer
gctttcactcatctcagccc

SEQ ID NO: 856 - DNA, 20 bp, Glycine max
  primer
aaggccaatgttgtttggag

SEQ ID NO: 857 - DNA, 20 bp, Glycine max
  primer
ccccatgtctgaccaagact

SEQ ID NO: 858 - DNA, 20 bp, Glycine max
  primer
gtggatcccaaaccacaaag

SEQ ID NO: 859 - DNA, 23 bp, Glycine max
  primer
tcggtgtactaatcagatgcaga

SEQ ID NO: 860 - DNA, 20 bp, Glycine max
  primer
tccatttccgagggctacta

SEQ ID NO: 861 - DNA, 21 bp, Glycine max
  primer
tttcttgatcacagaccctct

SEQ ID NO: 862 - DNA, 20 bp, Glycine max
  primer
tccctgaagaatagcaccca

SEQ ID NO: 863 - DNA, 20 bp, Glycine max
  primer
gcagggcagtatttacgcat

SEQ ID NO: 864 - DNA, 20 bp, Glycine max
  primer
tttgtggtaactgcgctttg

SEQ ID NO: 865 - DNA, 20 bp, Glycine max
  primer
tgggcattctcccacttatc

SEQ ID NO: 866 - DNA, 20 bp, Glycine max
  primer
tggctgcatggcatatagaa

SEQ ID NO: 867 - DNA, 20 bp, Glycine max
  primer
ttgcatgcacacttgcaata

SEQ ID NO: 868 - DNA, 20 bp, Glycine max
  primer
gcagctcacttccaagttcc

SEQ ID NO: 869 - DNA, 20 bp, Glycine max
  primer
tgcagaaggagcagaaggat

SEQ ID NO: 870 - DNA, 20 bp, Glycine max
  primer
gtaactgaaacggctcccaa

SEQ ID NO: 871 - DNA, 20 bp, Glycine max
  primer
gatcgtgagaaggaagcctg

SEQ ID NO: 872 - DNA, 20 bp, Glycine max
  primer
cttcaatgagcggggttcta

SEQ ID NO: 873 - DNA, 20 bp, Glycine max
  primer
gtgttggtttctcaggcgtt

SEQ ID NO: 874 - DNA, 20 bp, Glycine max
  primer
caacactctctggagcatcg

SEQ ID NO: 875 - DNA, 20 bp, Glycine max
  primer
ccactcatcagctaccccat

SEQ ID NO: 876 - DNA, 20 bp, Glycine max
  primer
taatttgatgttccctcgcc

SEQ ID NO: 877 - DNA, 20 bp, Glycine max
  primer
atggttgcatctcagcctct

SEQ ID NO: 878 - DNA, 20 bp, Glycine max
  primer
gagactgtctgaccaagggc

SEQ ID NO: 879 - DNA, 20 bp, Glycine max
  primer
ctcaatgccttcggcataat

SEQ ID NO: 880 - DNA, 20 bp, Glycine max
  primer
ggaaggcaatcgtggttaaa

SEQ ID NO: 881 - DNA, 20 bp, Glycine max
  primer
acaagggaagatggtgatcg

SEQ ID NO: 882 - DNA, 20 bp, Glycine max
  primer
attgccatcgttgtgttcaa

SEQ ID NO: 883 - DNA, 21 bp, Glycine max
  primer
atcattgtaggttggctggag

SEQ ID NO: 884 - DNA, 19 bp, Glycine max
  primer
atggaaaaactggcgcgaa

SEQ ID NO: 885 - DNA, 20 bp, Glycine max
  primer
gatgaccgaaaggttggaaa

SEQ ID NO: 886 - DNA, 20 bp, Glycine max
  primer
tgggtggtcttttaggcttg

SEQ ID NO: 887 - DNA, 20 bp, Glycine max
  primer
ttttgtgctggtgaaaggaa

SEQ ID NO: 888 - DNA, 20 bp, Glycine max
  primer
ttaagggtccatgccaaaag

SEQ ID NO: 889 - DNA, 20 bp, Glycine max
  primer
taaccgctcctgttcgactt

SEQ ID NO: 890 - DNA, 20 bp, Glycine max
  primer
gccgaaggcacatctagttc

SEQ ID NO: 891 - DNA, 20 bp, Glycine max
  primer
gcaggaagcgacacgttaat

SEQ ID NO: 892 - DNA, 20 bp, Glycine max
  primer
tctacccttgatccagtgcc

SEQ ID NO: 893 - DNA, 20 bp, Glycine max
  primer
tcagcaatttcagctcatgg

SEQ ID NO: 894 - DNA, 20 bp, Glycine max
  primer
ttccgtcggttccatatttc

SEQ ID NO: 895 - DNA, 20 bp, Glycine max
  primer
agtcaattcccgaaccacag

SEQ ID NO: 896 - DNA, 20 bp, Glycine max
  primer
actgagggagtcaagagcga

SEQ ID NO: 897 - DNA, 20 bp, Glycine max
  primer
ctgggccattgttgattttc

SEQ ID NO: 898 - DNA, 20 bp, Glycine max
  primer
gaataacgcagccagaggac

SEQ ID NO: 899 - DNA, 20 bp, Glycine max
  primer
tggttctgagcttgaagtgc

SEQ ID NO: 900 - DNA, 20 bp, Glycine max
  primer
caggtggaagaccaagcagt

SEQ ID NO: 901 - DNA, 20 bp, Glycine max
  primer
tgttgtagtcacctgctggc

SEQ ID NO: 902 - DNA, 20 bp, Glycine max
  primer
gcttttgatgggctgctatc

SEQ ID NO: 903 - DNA, 20 bp, Glycine max
  primer
caggtctaatggtgggtgct

SEQ ID NO: 904 - DNA, 20 bp, Glycine max
  primer
tgcaagtgaatgtcgggata

SEQ ID NO: 905 - DNA, 20 bp, Glycine max
  primer
gcaactgaacttccaaaggg

SEQ ID NO: 906 - DNA, 20 bp, Glycine max
  primer
attcattggtgggaattgga

SEQ ID NO: 907 - DNA, 20 bp, Glycine max
  primer
gttgtccaaggaacaggcat

SEQ ID NO: 908 - DNA, 20 bp, Glycine max
  primer
ccaaagcttgcttttgcttc

SEQ ID NO: 909 - DNA, 20 bp, Glycine max
  primer
ccaacaattgggaatgatcc

SEQ ID NO: 910 - DNA, 20 bp, Glycine max
  primer
aggaagtgttcgaagagcca

SEQ ID NO: 911 - DNA, 20 bp, Glycine max
  primer
tcattcaataatcagctgcg

SEQ ID NO: 912 - DNA, 21 bp, Glycine max
  primer
gatgaaggggtttgagtttga

SEQ ID NO: 913 - DNA, 20 bp, Glycine max
  primer
ttgacttttcattgacccga

SEQ ID NO: 914 - DNA, 20 bp, Glycine max
  primer
tcactcgattcgactagcca

SEQ ID NO: 915 - DNA, 20 bp, Glycine max
  primer
aaggaaagggagggaacaga

SEQ ID NO: 916 - DNA, 20 bp, Glycine max
  primer
agggatactgaaaaccgcct

SEQ ID NO: 917 - DNA, 20 bp, Glycine max
  primer
ccttctggttttcgcatcat

SEQ ID NO: 918 - DNA, 20 bp, Glycine max
  primer
caagtgcagaagccaaatca

SEQ ID NO: 919 - DNA, 20 bp, Glycine max
  primer
tcctccgagagaaggaacaa

SEQ ID NO: 920 - DNA, 20 bp, Glycine max
  primer
cgagtttcttggctaggctg

SEQ ID NO: 921 - DNA, 20 bp, Glycine max
  primer
atctttcccgttttctgggt

SEQ ID NO: 922 - DNA, 20 bp, Glycine max
  primer
ccctcgttctctgtgtggtt

SEQ ID NO: 923 - DNA, 20 bp, Glycine max
  primer
tgaacctgtggtttcgatga

SEQ ID NO: 924 - DNA, 20 bp, Glycine max
  primer
acgcagggtttttcattcag

SEQ ID NO: 925 - DNA, 19 bp, Glycine max
  primer
gaaacacggtcgttcctgc

SEQ ID NO: 926 - DNA, 19 bp, Glycine max
  primer
tcgttttccgctcacgcac

SEQ ID NO: 927 - DNA, 20 bp, Glycine max
  primer
cgtcaggtttcgaattggtt

SEQ ID NO: 928 - DNA, 20 bp, Glycine max
  primer
cgtcgttttcttgctccttc

SEQ ID NO: 929 - DNA, 20 bp, Glycine max
  primer
attttgtgtcagggctgagg

SEQ ID NO: 930 - DNA, 20 bp, Glycine max
  primer
tgcctcgcagttatcttgtg

SEQ ID NO: 931 - DNA, 20 bp, Glycine max
  primer
ccgagaggaagatttggcta

SEQ ID NO: 932 - DNA, 20 bp, Glycine max
  primer
ttccatctgcttggtcttcc

SEQ ID NO: 933 - DNA, 20 bp, Glycine max
  primer
ttcccctagaagctctgcaa

SEQ ID NO: 934 - DNA, 20 bp, Glycine max
  primer
aggtcttcgcttgatgagga

SEQ ID NO: 935 - DNA, 20 bp, Glycine max
  primer
tcatcaacggtactggctca

SEQ ID NO: 936 - DNA, 20 bp, Glycine max
  primer
ccagtgacgttggactgaga

SEQ ID NO: 937 - DNA, 20 bp, Glycine max
  primer
cgaacgttctggatggactt

SEQ ID NO: 938 - DNA, 20 bp, Glycine max
  primer
cgacgaagcatgtgaaaatc

SEQ ID NO: 939 - DNA, 20 bp, Glycine max
  primer
attgccattttcaagccatc

SEQ ID NO: 940 - DNA, 20 bp, Glycine max
  primer
tggagcaacagtacgccata

SEQ ID NO: 941 - DNA, 20 bp, Glycine max
  primer
atccctgtgcagttgattcc

SEQ ID NO: 942 - DNA, 20 bp, Glycine max
  primer
cactgattgaatggggtgtg

SEQ ID NO: 943 - DNA, 22 bp, Glycine max
  primer
gcaatgctaatctaatggcaca

SEQ ID NO: 944 - DNA, 21 bp, Glycine max
  primer
ttgtcacaccaacaacgaatg

SEQ ID NO: 945 - DNA, 20 bp, Glycine max
  primer
ttatcgggaagatggtccac

SEQ ID NO: 946 - DNA, 20 bp, Glycine max
  primer
aagagcaggatttgcagcat

SEQ ID NO: 947 - DNA, 20 bp, Glycine max
  primer
atgcagtttgtggtgatgga

SEQ ID NO: 948 - DNA, 20 bp, Glycine max
  primer
tagagcatgggatgggaaag

SEQ ID NO: 949 - DNA, 25 bp, Glycine max
  primer
tgaaccatatctagagactactact

SEQ ID NO: 950 - DNA, 22 bp, Glycine max
  primer
agcatacttcatacatagggca

SEQ ID NO: 951 - DNA, 20 bp, Glycine max
  primer
tctgctttaattgcagccct

SEQ ID NO: 952 - DNA, 20 bp, Glycine max
  primer
gcgacaccacttccctttta

SEQ ID NO: 953 - DNA, 20 bp, Glycine max
  primer
taatgaaccccgggtatgtc

SEQ ID NO: 954 - DNA, 20 bp, Glycine max
  primer
ggggagactttgtagggagg

SEQ ID NO: 955 - DNA, 20 bp, Glycine max
  primer
cacacatcacacgagcagaa

SEQ ID NO: 956 - DNA, 20 bp, Glycine max
  primer
ggtgtaagtggcagtggctt

SEQ ID NO: 957 - DNA, 20 bp, Glycine max
  primer
cacacatcacacgagcagaa

SEQ ID NO: 958 - DNA, 20 bp, Glycine max
  primer
ggtgtaagtggcagtggctt

SEQ ID NO: 959 - DNA, 22 bp, Glycine max
  primer
aagtctctgtgctcttgttgga

SEQ ID NO: 960 - DNA, 20 bp, Glycine max
  primer
tgatgataggatgggcacta

SEQ ID NO: 961 - DNA, 20 bp, Glycine max
  primer
cagctgaaggcggagataac

SEQ ID NO: 962 - DNA, 20 bp, Glycine max
  primer
tgagcatcgatgagtggaag

SEQ ID NO: 963 - DNA, 20 bp, Glycine max
  primer
atcgttgtcttcttcgctgg

SEQ ID NO: 964 - DNA, 20 bp, Glycine max
  primer
tccacctccaccttgttgat

SEQ ID NO: 965 - DNA, 20 bp, Glycine max
  primer
gcaccgacccttatattgga

SEQ ID NO: 966 - DNA, 20 bp, Glycine max
  primer
atcttgggtgtccaaaggtg

SEQ ID NO: 967 - DNA, 20 bp, Glycine max
  primer
acttcaacatccctcaacgc

SEQ ID NO: 968 - DNA, 20 bp, Glycine max
  primer
ggaaaacgacattgaacgct

SEQ ID NO: 969 - DNA, 20 bp, Glycine max
  primer
ctgaacttgcttttcgaggg

SEQ ID NO: 970 - DNA, 20 bp, Glycine max
  primer
tcatacagttcgtccggtca

SEQ ID NO: 971 - DNA, 20 bp, Glycine max
  primer
ttggcccaaatctccataag

SEQ ID NO: 972 - DNA, 20 bp, Glycine max
  primer
ctggccgggttaaaaagaat

SEQ ID NO: 973 - DNA, 21 bp, Glycine max
  primer
tttctccacctcatcatcctg

SEQ ID NO: 974 - DNA, 20 bp, Glycine max
  primer
cggaggatccaattccaagt

SEQ ID NO: 975 - DNA, 20 bp, Glycine max
  primer
gagagttgcactctgcggat

SEQ ID NO: 976 - DNA, 21 bp, Glycine max
  primer
cataaaccagaggaagaggca

SEQ ID NO: 977 - DNA, 20 bp, Glycine max
  primer
ccgccatctttaactggaaa

SEQ ID NO: 978 - DNA, 20 bp, Glycine max
  primer
tgttggtccatgtctggaaa

SEQ ID NO: 979 - DNA, 20 bp, Glycine max
  primer
ggccacaaattctacatcca

SEQ ID NO: 980 - DNA, 21 bp, Glycine max
  primer
tggagggtgagtcattgttgt

SEQ ID NO: 981 - DNA, 20 bp, Glycine max
  primer
aggctcaagccttgtctctg

SEQ ID NO: 982 - DNA, 20 bp, Glycine max
  primer
accaccccatcaagatcaaa

SEQ ID NO: 983 - DNA, 21 bp, Glycine max
  primer
tccctttttcatccagaatcc

SEQ ID NO: 984 - DNA, 20 bp, Glycine max
  primer
cccttttaatgcatgctcgt

SEQ ID NO: 985 - DNA, 20 bp, Glycine max
  primer
gtttcacggaggagcaagag

SEQ ID NO: 986 - DNA, 20 bp, Glycine max
  primer
cggtgtcgaggaaattctgt

SEQ ID NO: 987 - DNA, 20 bp, Glycine max
  primer
ggggttacacacctacacgg

SEQ ID NO: 988 - DNA, 20 bp, Glycine max
  primer
ccaccactgatcttgagggt

SEQ ID NO: 989 - DNA, 22 bp, Glycine max
  primer
caaaaaccaaagaagagttgcc

SEQ ID NO: 990 - DNA, 25 bp, Glycine max
  primer
cactagctatgtagttcataagacg

SEQ ID NO: 991 - DNA, 20 bp, Glycine max
  primer
gccgccagaaagaaacttag

SEQ ID NO: 992 - DNA, 20 bp, Glycine max
  primer
gcttcgccaaagcttgaata

SEQ ID NO: 993 - DNA, 20 bp, Glycine max
  primer
tcttcgtcgccaaattcttt

SEQ ID NO: 994 - DNA, 20 bp, Glycine max
  primer
cagcgactgaaacagagcag

SEQ ID NO: 995 - DNA, 20 bp, Glycine max
  primer
tggctctttgagcacttcct

SEQ ID NO: 996 - DNA, 20 bp, Glycine max
  primer
caatttgccacctggttttt

SEQ ID NO: 997 - DNA, 20 bp, Glycine max
  primer
gagtctgcaggcctcgttat

SEQ ID NO: 998 - DNA, 20 bp, Glycine max
  primer
aacgaagccttacgaaagca

SEQ ID NO: 999 - DNA, 20 bp, Glycine max
  primer
cggaaccagaaactacaggc

SEQ ID NO: 1000 - DNA, 20 bp, Glycine max
  primer
attgctccatgaaccctcag

SEQ ID NO: 1001 - DNA, 20 bp, Glycine max
  primer
gaagcggtccatgtcgttat

SEQ ID NO: 1002 - DNA, 20 bp, Glycine max
  primer
gaagaccccatcatcggata

SEQ ID NO: 1003 - DNA, 20 bp, Glycine max
  primer
ttcttcagatccacccgttc

SEQ ID NO: 1004 - DNA, 20 bp, Glycine max
  primer
cacacgttccatacccagtg

SEQ ID NO: 1005 - DNA, 20 bp, Glycine max
  primer
gagactggctctctgggttg

SEQ ID NO: 1006 - DNA, 20 bp, Glycine max
  primer
aagacaggggaatacagggg

SEQ ID NO: 1007 - DNA, 20 bp, Glycine max
  primer
tgcacccagttgtcatcaat

SEQ ID NO: 1008 - DNA, 20 bp, Glycine max
  primer
ttgagcagcatccaatcaag

SEQ ID NO: 1009 - DNA, 20 bp, Glycine max
  primer
ggttttggccagtggaatta

SEQ ID NO: 1010 - DNA, 20 bp, Glycine max
  primer
catcagggactccttttcca

SEQ ID NO: 1011 - DNA, 20 bp, Glycine max
  primer
gttgcagattgtgccgtatg

SEQ ID NO: 1012 - DNA, 20 bp, Glycine max
  primer
cccagactcacttctctggc

SEQ ID NO: 1013 - DNA, 20 bp, Glycine max
  primer
cgccattttctttacctcca

SEQ ID NO: 1014 - DNA, 20 bp, Glycine max
  primer
ggaatttgtgtcccctgaaa

SEQ ID NO: 1015 - DNA, 20 bp, Glycine max
  primer
gatgactcccctgctgaaaa

SEQ ID NO: 1016 - DNA, 20 bp, Glycine max
  primer
gcttgctacagggaaacacc

SEQ ID NO: 1017 - DNA, 20 bp, Glycine max
  primer
gtggttccaccattgcttct

SEQ ID NO: 1018 - DNA, 20 bp, Glycine max
  primer
aaaacttgggcatgttcagc

SEQ ID NO: 1019 - DNA, 20 bp, Glycine max
  primer
cctgcgactgcattgaacta

SEQ ID NO: 1020 - DNA, 20 bp, Glycine max
  primer
gagagtatccggcgtcacat

SEQ ID NO: 1021 - DNA, 20 bp, Glycine max
  primer
tgaaaagggagacgaatgct

SEQ ID NO: 1022 - DNA, 20 bp, Glycine max
  primer
tgattcttgtacggtggctg

SEQ ID NO: 1023 - DNA, 20 bp, Glycine max
  primer
aagcgaaggactcagactcg

SEQ ID NO: 1024 - DNA, 20 bp, Glycine max
  primer
cgacgagtagaacgcagtga

SEQ ID NO: 1025 - DNA, 24 bp, Glycine max
  primer
ggaaactggtcatggtaagtagaa

SEQ ID NO: 1026 - DNA, 19 bp, Glycine max
  primer
ccaccagcttgagtcatgg

SEQ ID NO: 1027 - DNA, 22 bp, Glycine max
  primer
tccttgccttacgctagtcttt

SEQ ID NO: 1028 - DNA, 22 bp, Glycine max
  primer
tgacaacaagcttcaaaggaga

SEQ ID NO: 1029 - DNA, 21 bp, Glycine max
  primer
gaaggaatgtatctgatgggg

SEQ ID NO: 1030 - DNA, 22 bp, Glycine max
  primer
ttgtgtttcagaatatggcctg

SEQ ID NO: 1031 - DNA, 20 bp, Glycine max
  primer
aggttgctttagtctccgca

SEQ ID NO: 1032 - DNA, 20 bp, Glycine max
  primer
ccaagggaaagaacaggaca

SEQ ID NO: 1033 - DNA, 20 bp, Glycine max
  primer
agtcgccacggagatatgat

SEQ ID NO: 1034 - DNA, 20 bp, Glycine max
  primer
tatgtggtagtgcgtgggag

SEQ ID NO: 1035 - DNA, 20 bp, Glycine max
  primer
tcacaagccttgcacttttg

SEQ ID NO: 1036 - DNA, 20 bp, Glycine max
  primer
ttggaatgggtggtgaattt

SEQ ID NO: 1037 - DNA, 20 bp, Glycine max
  primer
cacgggacattcaacatctg

SEQ ID NO: 1038 - DNA, 20 bp, Glycine max
  primer
tgccattgtttatgctccaa

SEQ ID NO: 1039 - DNA, 20 bp, Glycine max
  primer
tctccacaagttcaagcacg

SEQ ID NO: 1040 - DNA, 20 bp, Glycine max
  primer
accagcagctctgggattta

SEQ ID NO: 1041 - DNA, 20 bp, Glycine max
  primer
tctttgggtggaaatcaagg

SEQ ID NO: 1042 - DNA, 20 bp, Glycine max
  primer
cgtttgatacaactgtgcgg

SEQ ID NO: 1043 - DNA, 20 bp, Glycine max
  primer
cctcttttgccatttgggta

SEQ ID NO: 1044 - DNA, 21 bp, Glycine max
  primer
tgaaacaggatacaacagggg

SEQ ID NO: 1045 - DNA, 20 bp, Glycine max
  primer
gcatcacatgtccctcacac

SEQ ID NO: 1046 - DNA, 20 bp, Glycine max
  primer
ttaaggctgagccgttgact

SEQ ID NO: 1047 - DNA, 20 bp, Glycine max
  primer
gcaagctcactcgctttctt

SEQ ID NO: 1048 - DNA, 20 bp, Glycine max
  primer
taagaagaccaaaggtcggc

SEQ ID NO: 1049 - DNA, 20 bp, Glycine max
  primer
ccacggagaagattcgtgag

SEQ ID NO: 1050 - DNA, 22 bp, Glycine max
  primer
tgcttaagctctctccatcaga

SEQ ID NO: 1051 - DNA, 20 bp, Glycine max
  primer
agaaggtgtgggaaacatgc

SEQ ID NO: 1052 - DNA, 20 bp, Glycine max
  primer
gctgttttaggctagctgcg

SEQ ID NO: 1053 - DNA, 20 bp, Glycine max
  primer
atttgacttctggggagcct

SEQ ID NO: 1054 - DNA, 20 bp, Glycine max
  primer
gaccccacaagagcaagaag

SEQ ID NO: 1055 - DNA, 20 bp, Glycine max
  primer
gaccccacaagagcaagaag

SEQ ID NO: 1056 - DNA, 20 bp, Glycine max
  primer
atttgacttctggggagcct

SEQ ID NO: 1057 - DNA, 20 bp, Glycine max
  primer
gcataagatccactgcacca

SEQ ID NO: 1058 - DNA, 20 bp, Glycine max
  primer
acacggcagacacttacagc

SEQ ID NO: 1059 - DNA, 20 bp, Glycine max
  primer
tggaggggagtacgagtctg

SEQ ID NO: 1060 - DNA, 20 bp, Glycine max
  primer
taggatggcttggctgtagg

SEQ ID NO: 1061 - DNA, 20 bp, Glycine max
  primer
gacgaagaggattacgacgg

SEQ ID NO: 1062 - DNA, 20 bp, Glycine max
  primer
aggccggacattcaactcta

SEQ ID NO: 1063 - DNA, 21 bp, Glycine max
  primer
cgtggtgatgaaatggatctt

SEQ ID NO: 1064 - DNA, 20 bp, Glycine max
  primer
ggagttggggttccttcatt

SEQ ID NO: 1065 - DNA, 20 bp, Glycine max
  primer
gatactccagaacgggacga

SEQ ID NO: 1066 - DNA, 20 bp, Glycine max
  primer
gctatgctgatgctcagtcg

SEQ ID NO: 1067 - DNA, 20 bp, Glycine max
  primer
atgctttggccaatgtgaat

SEQ ID NO: 1068 - DNA, 20 bp, Glycine max
  primer
tcttcgttggcatggtcata

SEQ ID NO: 1069 - DNA, 20 bp, Glycine max
  primer
gaatggattccgatgattgc

SEQ ID NO: 1070 - DNA, 20 bp, Glycine max
  primer
tatgcaagagatcagcacgc

SEQ ID NO: 1071 - DNA, 20 bp, Glycine max
  primer
tcaagggttgagtgtgcaag

SEQ ID NO: 1072 - DNA, 20 bp, Glycine max
  primer
cgtggtgacacggtctattg

SEQ ID NO: 1073 - DNA, 20 bp, Glycine max
  primer
attcctgcattagggaacca

SEQ ID NO: 1074 - DNA, 20 bp, Glycine max
  primer
aagcaagttccccaggctac

SEQ ID NO: 1075 - DNA, 20 bp, Glycine max
  primer
ttgttgtggttttgcagctc

SEQ ID NO: 1076 - DNA, 21 bp, Glycine max
  primer
cgagggtagattggagaaagg

SEQ ID NO: 1077 - DNA, 20 bp, Glycine max
  primer
gtgctgatgacagaacgcat

SEQ ID NO: 1078 - DNA, 20 bp, Glycine max
  primer
tgcgatccatccacaattta

SEQ ID NO: 1079 - DNA, 20 bp, Glycine max
  primer
agtacgagttttgcagcggt

SEQ ID NO: 1080 - DNA, 20 bp, Glycine max
  primer
gcttcctttgttgccacatt

SEQ ID NO: 1081 - DNA, 20 bp, Glycine max
  primer
gtctgtcaaggcgagaaagc

SEQ ID NO: 1082 - DNA, 20 bp, Glycine max
  primer
ccgaagctcctcaatctgtc

SEQ ID NO: 1083 - DNA, 20 bp, Glycine max
  primer
ccttgtgtggagttgaagca

SEQ ID NO: 1084 - DNA, 20 bp, Glycine max
  primer
ggagtgtgccaatacagggt

SEQ ID NO: 1085 - DNA, 20 bp, Glycine max
  primer
ctaccaatcgccaagtcaca

SEQ ID NO: 1086 - DNA, 20 bp, Glycine max
  primer
cgtccacggctagagaaaac

SEQ ID NO: 1087 - DNA, 21 bp, Glycine max
  primer
aaccctattgaacacccttga

SEQ ID NO: 1088 - DNA, 22 bp, Glycine max
  primer
ttctgcatacactcatgcaaca

SEQ ID NO: 1089 - DNA, 20 bp, Glycine max
  primer
tatttcctttcgcaggatgc

SEQ ID NO: 1090 - DNA, 20 bp, Glycine max
  primer
gcattcagggattcaaggat

SEQ ID NO: 1091 - DNA, 20 bp, Glycine max
  primer
gctgaacacgagaaagcaca

SEQ ID NO: 1092 - DNA, 20 bp, Glycine max
  primer
taacagggaagaaattgcgg

SEQ ID NO: 1093 - DNA, 20 bp, Glycine max
  primer
cgggtacgaatttgcttgag

SEQ ID NO: 1094 - DNA, 20 bp, Glycine max
  primer
ttgcagagaaaccataggca

SEQ ID NO: 1095 - DNA, 20 bp, Glycine max
  primer
ttggaaaattgggagtgagg

SEQ ID NO: 1096 - DNA, 20 bp, Glycine max
  primer
accggcataagatccacaac

SEQ ID NO: 1097 - DNA, 20 bp, Glycine max
  primer
ttctttgggggttgaagttg

SEQ ID NO: 1098 - DNA, 20 bp, Glycine max
  primer
ccgctccaagaaaaattctg

SEQ ID NO: 1099 - DNA, 20 bp, Glycine max
  primer
agagcttgtggaattccctg

SEQ ID NO: 1100 - DNA, 20 bp, Glycine max
  primer
agcatccaattcaaggaaca

SEQ ID NO: 1101 - DNA, 20 bp, Glycine max
  primer
ttggatttgtgatgccgtta

SEQ ID NO: 1102 - DNA, 20 bp, Glycine max
  primer
catcataggaagggaggcaa

SEQ ID NO: 1103 - DNA, 20 bp, Glycine max
  primer
ttcttttcaagcaacgctga

SEQ ID NO: 1104 - DNA, 20 bp, Glycine max
  primer
agtagtgggcactcgtcacc

SEQ ID NO: 1105 - DNA, 20 bp, Glycine max
  primer
atcagcagtcaagagcacca

SEQ ID NO: 1106 - DNA, 20 bp, Glycine max
  primer
caaattgcagacacgatgct

SEQ ID NO: 1107 - DNA, 20 bp, Glycine max
  primer
ggttcttggactgttgaccg

SEQ ID NO: 1108 - DNA, 21 bp, Glycine max
  primer
gaaatgcaagtaatttccccc

SEQ ID NO: 1109 - DNA, 20 bp, Glycine max
  primer
acacctttgtccaccgattc

SEQ ID NO: 1110 - DNA, 20 bp, Glycine max
  primer
tccgtccaccaagaaaaatc

SEQ ID NO: 1111 - DNA, 20 bp, Glycine max
  primer
tgccgaattcaatgataccc

SEQ ID NO: 1112 - DNA, 20 bp, Glycine max
  primer
tggcatgcatttctggtatg

SEQ ID NO: 1113 - DNA, 20 bp, Glycine max
  primer
ctgtcaacggaaagtgcaga

SEQ ID NO: 1114 - DNA, 20 bp, Glycine max
  primer
ctgcatcaccaaaaccattg

SEQ ID NO: 1115 - DNA, 20 bp, Glycine max
  primer
gccactcctttcaggaagtt

SEQ ID NO: 1116 - DNA, 23 bp, Glycine max
  primer
cccaagttcttatgtgaataccc

SEQ ID NO: 1117 - DNA, 21 bp, Glycine max
  primer
tgcatttactagatcacgggg

SEQ ID NO: 1118 - DNA, 21 bp, Glycine max
  primer
tggaatatctgcaacaggatg

SEQ ID NO: 1119 - DNA, 20 bp, Glycine max
  primer
gcatcgagaaggaaaacgaa

SEQ ID NO: 1120 - DNA, 20 bp, Glycine max
  primer
ttcctctgatttttccccag

SEQ ID NO: 1121 - DNA, 20 bp, Glycine max
  primer
cgttgttcctttggcaattt

SEQ ID NO: 1122 - DNA, 20 bp, Glycine max
  primer
cttccatgcagatgatgcac

SEQ ID NO: 1123 - DNA, 20 bp, Glycine max
  primer
taggcacagtttcacatggc

SEQ ID NO: 1124 - DNA, 20 bp, Glycine max
  primer
atccaccatcccagaatcaa

SEQ ID NO: 1125 - DNA, 20 bp, Glycine max
  primer
gtttggcgtcttggtttgat

SEQ ID NO: 1126 - DNA, 20 bp, Glycine max
  primer
aagaagaggctgccacaaaa

SEQ ID NO: 1127 - DNA, 20 bp, Glycine max
  primer
cttggagggttatgttccca

SEQ ID NO: 1128 - DNA, 20 bp, Glycine max
  primer
gtctaaaacgaacgggcaaa

SEQ ID NO: 1129 - DNA, 20 bp, Glycine max
  primer
gttactgggaagcaagtgcc

SEQ ID NO: 1130 - DNA, 21 bp, Glycine max
  primer
tcaattcccaagaagagagca

SEQ ID NO: 1131 - DNA, 20 bp, Glycine max
  primer
agcagtggcaacaacaacag

SEQ ID NO: 1132 - DNA, 20 bp, Glycine max
  primer
agttgaggtgctggaaagga

SEQ ID NO: 1133 - DNA, 20 bp, Glycine max
  primer
cttttgcagtagcatcaccg

SEQ ID NO: 1134 - DNA, 20 bp, Glycine max
  primer
tgtgacatggaacacaccaa

SEQ ID NO: 1135 - DNA, 20 bp, Glycine max
  primer
gccatatgcaaatgcagaaa

SEQ ID NO: 1136 - DNA, 20 bp, Glycine max
  primer
agcagctgcaatagctgtca

SEQ ID NO: 1137 - DNA, 20 bp, Glycine max
  primer
gccgttaagaaccactggaa

SEQ ID NO: 1138 - DNA, 20 bp, Glycine max
  primer
ggaggagcaagagtcaatgc

SEQ ID NO: 1139 - DNA, 20 bp, Glycine max
  primer
ttcccctctaattcaacccc

SEQ ID NO: 1140 - DNA, 20 bp, Glycine max
  primer
tctcctgtgaggcaactcct

SEQ ID NO: 1141 - DNA, 20 bp, Glycine max
  primer
aagcacttacccatgcgaac

SEQ ID NO: 1142 - DNA, 20 bp, Glycine max
  primer
cttgagggatccacagcatt

SEQ ID NO: 1143 - DNA, 20 bp, Glycine max
  primer
tcctttctcttttggtggga

SEQ ID NO: 1144 - DNA, 20 bp, Glycine max
  primer
gggtccgtacaaggaacaga

SEQ ID NO: 1145 - DNA, 20 bp, Glycine max
  primer
aggaccttttcattggcctt

SEQ ID NO: 1146 - DNA, 20 bp, Glycine max
  primer
atcatcatgctcttccggtc

SEQ ID NO: 1147 - DNA, 20 bp, Glycine max
  primer
ttctccagtgttcccgtttc

SEQ ID NO: 1148 - DNA, 20 bp, Glycine max
  primer
tgcagttggtttcagcactt

SEQ ID NO: 1149 - DNA, 20 bp, Glycine max
  primer
tttcatcaggcaaagcaatg

SEQ ID NO: 1150 - DNA, 20 bp, Glycine max
  primer
gcagtgtcagctgcttcatc

SEQ ID NO: 1151 - DNA, 20 bp, Glycine max
  primer
taaatgaagagggcccatga

SEQ ID NO: 1152 - DNA, 20 bp, Glycine max
  primer
cgtcgtgaatggataagcaa

SEQ ID NO: 1153 - DNA, 22 bp, Glycine max
  primer
tgcagtctggttgcataatagc

SEQ ID NO: 1154 - DNA, 20 bp, Glycine max
  primer
cgtcgtttttcaggcaagat

SEQ ID NO: 1155 - DNA, 20 bp, Glycine max
  primer
cacgaaatttggtccctcat

SEQ ID NO: 1156 - DNA, 20 bp, Glycine max
  primer
gggtaagctgattgcaccat

SEQ ID NO: 1157 - DNA, 20 bp, Glycine max
  primer
cctggaagaaccgataacga

SEQ ID NO: 1158 - DNA, 20 bp, Glycine max
  primer
tgagtttgagggtcgattcc

SEQ ID NO: 1159 - DNA, 21 bp, Glycine max
  primer
caatgagaacaccccttttga

SEQ ID NO: 1160 - DNA, 20 bp, Glycine max
  primer
ctccagaatgtggtgggaat

SEQ ID NO: 1161 - DNA, 20 bp, Glycine max
  primer
cagaatacagctcgtgccaa

SEQ ID NO: 1162 - DNA, 20 bp, Glycine max
  primer
tgaccaagtttggaccccta

SEQ ID NO: 1163 - DNA, 20 bp, Glycine max
  primer
gccccaaagagatcaacaaa

SEQ ID NO: 1164 - DNA, 20 bp, Glycine max
  primer
ccgcatctctttaaacctgc

SEQ ID NO: 1165 - DNA, 24 bp, Glycine max
  primer
tcagctgataagaatcagacttgt

SEQ ID NO: 1166 - DNA, 21 bp, Glycine max
  primer
tttccaagctgatagaacgct

SEQ ID NO: 1167 - DNA, 20 bp, Glycine max
  primer
agtggcagtgcaattcacaa

SEQ ID NO: 1168 - DNA, 20 bp, Glycine max
  primer
tgtccaaccacccttagcac

SEQ ID NO: 1169 - DNA, 20 bp, Glycine max
  primer
tgaagtgcatcatgctttgg

SEQ ID NO: 1170 - DNA, 20 bp, Glycine max
  primer
tcctccatcttctccctcct

SEQ ID NO: 1171 - DNA, 20 bp, Glycine max
  primer
aatagctgggagattgcctg

SEQ ID NO: 1172 - DNA, 20 bp, Glycine max
  primer
gggtcaatgcctttgctaat

SEQ ID NO: 1173 - DNA, 20 bp, Glycine max
  primer
aaccacatgattgattgcca

SEQ ID NO: 1174 - DNA, 21 bp, Glycine max
  primer
tctggttactcgtagcatcgc

SEQ ID NO: 1175 - DNA, 20 bp, Glycine max
  primer
ttaccacctcaagagccacc

SEQ ID NO: 1176 - DNA, 20 bp, Glycine max
  primer
agccgaagctctcataccaa

SEQ ID NO: 1177 - DNA, 19 bp, Glycine max
  primer
tggtgctccagcaacaact

SEQ ID NO: 1178 - DNA, 20 bp, Glycine max
  primer
accccagtgatgaaccttcc

SEQ ID NO: 1179 - DNA, 20 bp, Glycine max
  primer
gcttttgctttgctttgctt

SEQ ID NO: 1180 - DNA, 20 bp, Glycine max
  primer
agggacacagatccgagatg

SEQ ID NO: 1181 - DNA, 20 bp, Glycine max
  primer
tgtgtaccaaacgaatccga

SEQ ID NO: 1182 - DNA, 20 bp, Glycine max
  primer
tgggaacatgatggtgagaa

SEQ ID NO: 1183 - DNA, 20 bp, Glycine max
  primer
cttggcatctttgtgtccct

SEQ ID NO: 1184 - DNA, 20 bp, Glycine max
  primer
cattctggtgctttgtccac

SEQ ID NO: 1185 - DNA, 20 bp, Glycine max
  primer
ctgcatcaccaaaaccattg

SEQ ID NO: 1186 - DNA, 20 bp, Glycine max
  primer
ttcatcatcggaaagtgcag

SEQ ID NO: 1187 - DNA, 20 bp, Glycine max
  primer
tgtcaaaccgcttaacacca

SEQ ID NO: 1188 - DNA, 20 bp, Glycine max
  primer
gtgcaagatattccccatgc

SEQ ID NO: 1189 - DNA, 20 bp, Glycine max
  primer
caagctcgtcattttgctca

SEQ ID NO: 1190 - DNA, 20 bp, Glycine max
  primer
tcaagctaccgaactcccat

SEQ ID NO: 1191 - DNA, 20 bp, Glycine max
  primer
aatcccttgaattggaaccc

SEQ ID NO: 1192 - DNA, 20 bp, Glycine max
  primer
ttccaaggacatccagaagc

SEQ ID NO: 1193 - DNA, 20 bp, Glycine max
  primer
tgtggtgattctcgtccatc

SEQ ID NO: 1194 - DNA, 20 bp, Glycine max
  primer
gctgctggaaacctttctga

SEQ ID NO: 1195 - DNA, 20 bp, Glycine max
  primer
aaagatgttgctgccgactt

SEQ ID NO: 1196 - DNA, 20 bp, Glycine max
  primer
agcacacacctgtggtcaga

SEQ ID NO: 1197 - DNA, 20 bp, Glycine max
  primer
catcctcttctttgatccgc

SEQ ID NO: 1198 - DNA, 20 bp, Glycine max
  primer
gtgctccactgaaagttgcc

SEQ ID NO: 1199 - DNA, 20 bp, Glycine max
  primer
caccccaaaagtccttcaaa

SEQ ID NO: 1200 - DNA, 20 bp, Glycine max
  primer
aagcggatccatgtttatgc

SEQ ID NO: 1201 - DNA, 20 bp, Glycine max
  primer
tcagacttgggttcctcctc

SEQ ID NO: 1202 - DNA, 20 bp, Glycine max
  primer
acccaaacgtacccatttga

SEQ ID NO: 1203 - DNA, 20 bp, Glycine max
  primer
agatgggtcaccattcttgc

SEQ ID NO: 1204 - DNA, 20 bp, Glycine max
  primer
catagccgtgagtggtgatg

SEQ ID NO: 1205 - DNA, 20 bp, Glycine max
  primer
agaagctccttggcaaacaa

SEQ ID NO: 1206 - DNA, 20 bp, Glycine max
  primer
tgacatcttgcttctgctgg

SEQ ID NO: 1207 - DNA, 20 bp, Glycine max
  primer
cctgttgcatactcttcgca

SEQ ID NO: 1208 - DNA, 19 bp, Glycine max
  primer
agggtcattggaggacgac

SEQ ID NO: 1209 - DNA, 20 bp, Glycine max
  primer
ccaaaagttcttggggaaca

SEQ ID NO: 1210 - DNA, 20 bp, Glycine max
  primer
tggcgtgatgttaagctttg

SEQ ID NO: 1211 - DNA, 20 bp, Glycine max
  primer
tccaaatggggaaataggtt

SEQ ID NO: 1212 - DNA, 21 bp, Glycine max
  primer
tgagtgatgatgattggaagg

SEQ ID NO: 1213 - DNA, 20 bp, Glycine max
  primer
accaaatggaagtttgtcgc

SEQ ID NO: 1214 - DNA, 20 bp, Glycine max
  primer
cccagcttcttcctcagatg

SEQ ID NO: 1215 - DNA, 20 bp, Glycine max
  primer
tcagctcagaatcagccaaa

SEQ ID NO: 1216 - DNA, 20 bp, Glycine max
  primer
atcaatgcttcctccatcca

SEQ ID NO: 1217 - DNA, 20 bp, Glycine max
  primer
atttgttgaggcaggagctg

SEQ ID NO: 1218 - DNA, 20 bp, Glycine max
  primer
aggaaacctggtgcacaatc

SEQ ID NO: 1219 - DNA, 20 bp, Glycine max
  primer
tccttttctcttcgcttggt

SEQ ID NO: 1220 - DNA, 20 bp, Glycine max
  primer
ataacggtggccttcagaac

SEQ ID NO: 1221 - DNA, 20 bp, Glycine max
  primer
ctcctgtggtttgcttgtga

SEQ ID NO: 1222 - DNA, 20 bp, Glycine max
  primer
tttctcttgatgaaagggca

SEQ ID NO: 1223 - DNA, 20 bp, Glycine max
  primer
tgtgaggcacatttaggcag

SEQ ID NO: 1224 - DNA, 20 bp, Glycine max
  primer
gcttttatggtgatggggaa

SEQ ID NO: 1225 - DNA, 20 bp, Glycine max
  primer
tggacttggtgagtttggtg

SEQ ID NO: 1226 - DNA, 22 bp, Glycine max
  primer
tgttgaatagatcaagggcaga

SEQ ID NO: 1227 - DNA, 20 bp, Glycine max
  primer
cccattcatatggccacttc

SEQ ID NO: 1228 - DNA, 20 bp, Glycine max
  primer
gggggtgggtttaggaataa

SEQ ID NO: 1229 - DNA, 20 bp, Glycine max
  primer
ttggatttccggtacagagg

SEQ ID NO: 1230 - DNA, 20 bp, Glycine max
  primer
tttgaaaatccattccagcc

SEQ ID NO: 1231 - DNA, 20 bp, Glycine max
  primer
atctcttacgctttgcagcc

SEQ ID NO: 1232 - DNA, 20 bp, Glycine max
  primer
ggcatctgcaacaactctga

SEQ ID NO: 1233 - DNA, 21 bp, Glycine max
  primer
tggctttttatcttgcgtctg

SEQ ID NO: 1234 - DNA, 19 bp, Glycine max
  primer
acaaagcaacccaggaaat

SEQ ID NO: 1235 - DNA, 20 bp, Glycine max
  primer
cccctagctagtgtgaccca

SEQ ID NO: 1236 - DNA, 22 bp, Glycine max
  primer
ctcgctatcctattggatgttt

SEQ ID NO: 1237 - DNA, 20 bp, Glycine max
  primer
gctgtcttcaccggacctta

SEQ ID NO: 1238 - DNA, 20 bp, Glycine max
  primer
gctccagttggtacttcgga

SEQ ID NO: 1239 - DNA, 20 bp, Glycine max
  primer
tccggtggtgtaatcagctt

SEQ ID NO: 1240 - DNA, 20 bp, Glycine max
  primer
tgcatgggctgaaactatga

SEQ ID NO: 1241 - DNA, 20 bp, Glycine max
  primer
tgaacttgcagactttggga

SEQ ID NO: 1242 - DNA, 20 bp, Glycine max
  primer
aagcaatccaaagggctagg

SEQ ID NO: 1243 - DNA, 20 bp, Glycine max
  primer
actttgcgaaaagcaaggaa

SEQ ID NO: 1244 - DNA, 20 bp, Glycine max
  primer
tgacagattgcctatgctgg

SEQ ID NO: 1245 - DNA, 20 bp, Glycine max
  primer
ctgttgaggaactgcctgtg

SEQ ID NO: 1246 - DNA, 21 bp, Glycine max
  primer
ggctaatttgctccctaattg

SEQ ID NO: 1247 - DNA, 20 bp, Glycine max
  primer
tggaccaggaatatgcacaa

SEQ ID NO: 1248 - DNA, 20 bp, Glycine max
  primer
tcccgagacaggatgagaac

SEQ ID NO: 1249 - DNA, 21 bp, Glycine max
  primer
caccttccgtgaaagaggtaa

SEQ ID NO: 1250 - DNA, 22 bp, Glycine max
  primer
gccattagtctgttttccatca

SEQ ID NO: 1251 - DNA, 20 bp, Glycine max
  primer
caagagaaggaggaaagccc

SEQ ID NO: 1252 - DNA, 20 bp, Glycine max
  primer
ggtcctcactgaagaagcca

SEQ ID NO: 1253 - DNA, 20 bp, Glycine max
  primer
tgttgttgccaccatcactt

SEQ ID NO: 1254 - DNA, 20 bp, Glycine max
  primer
tggaacacccatctaagcaa

SEQ ID NO: 1255 - DNA, 20 bp, Glycine max
  primer
aagccagagacattccagtg

SEQ ID NO: 1256 - DNA, 21 bp, Glycine max
  primer
agttactgaacggggattaaa

SEQ ID NO: 1257 - DNA, 22 bp, Glycine max
  primer
ttccactctcctacttagcctg

SEQ ID NO: 1258 - DNA, 20 bp, Glycine max
  primer
tccaagatgatgccatttga

SEQ ID NO: 1259 - DNA, 20 bp, Glycine max
  primer
cttgcctcttaggccctctt

SEQ ID NO: 1260 - DNA, 20 bp, Glycine max
  primer
cttgccttggttttccatgt

SEQ ID NO: 1261 - DNA, 20 bp, Glycine max
  primer
cctccaggcaagagtcaatc

SEQ ID NO: 1262 - DNA, 20 bp, Glycine max
  primer
cgtcgtctcttcttgcattg

SEQ ID NO: 1263 - DNA, 20 bp, Glycine max
  primer
agagccggagtagcagatga

SEQ ID NO: 1264 - DNA, 20 bp, Glycine max
  primer
atggcttcagggtttgattg

SEQ ID NO: 1265 - DNA, 20 bp, Glycine max
  primer
tcctgtctttttggtgggag

SEQ ID NO: 1266 - DNA, 20 bp, Glycine max
  primer
cggggtctgtacaaggaaca

SEQ ID NO: 1267 - DNA, 20 bp, Glycine max
  primer
agcattgttgattgatgggc

SEQ ID NO: 1268 - DNA, 20 bp, Glycine max
  primer
atcactgtgaatgggccaaa

SEQ ID NO: 1269 - DNA, 20 bp, Glycine max
  primer
ttgaactttgaagtgccgtg

SEQ ID NO: 1270 - DNA, 22 bp, Glycine max
  primer
ttttgatttcctgtctcactgg

SEQ ID NO: 1271 - DNA, 20 bp, Glycine max
  primer
aaggaggtttacagcgctca

SEQ ID NO: 1272 - DNA, 20 bp, Glycine max
  primer
aatcaatctgtttgtggcgg

SEQ ID NO: 1273 - DNA, 21 bp, Glycine max
  primer
aacttggcctctaatgaggga

SEQ ID NO: 1274 - DNA, 20 bp, Glycine max
  primer
ccccttatgggtcctgaagt

SEQ ID NO: 1275 - DNA, 20 bp, Glycine max
  primer
tccttccccctctagtcaca

SEQ ID NO: 1276 - DNA, 21 bp, Glycine max
  primer
ccaaaagtaactccaatgcca

SEQ ID NO: 1277 - DNA, 20 bp, Glycine max
  primer
catggcaatttcgaggtctt

SEQ ID NO: 1278 - DNA, 20 bp, Glycine max
  primer
ctcgtagccgtatcaaggaa

SEQ ID NO: 1279 - DNA, 20 bp, Glycine max
  primer
aaaatgccttggcaattcac

SEQ ID NO: 1280 - DNA, 20 bp, Glycine max
  primer
ccaaggttttccctggtaca

SEQ ID NO: 1281 - DNA, 20 bp, Glycine max
  primer
gcactgagacacctgaatcg

SEQ ID NO: 1282 - DNA, 20 bp, Glycine max
  primer
tttgggcaccagtttttctc

SEQ ID NO: 1283 - DNA, 20 bp, Glycine max
  primer
tgcagcaaagttgttgaagg

SEQ ID NO: 1284 - DNA, 20 bp, Glycine max
  primer
aagggttggatgaaaaaccc

SEQ ID NO: 1285 - DNA, 20 bp, Glycine max
  primer
gggtggatgaaaaacacacc

SEQ ID NO: 1286 - DNA, 20 bp, Glycine max
  primer
agtgcttgttgtgcttccct

SEQ ID NO: 1287 - DNA, 20 bp, Glycine max
  primer
gcagggagtgaatcaaccat

SEQ ID NO: 1288 - DNA, 20 bp, Glycine max
  primer
gagtcttcgaaaaggagggg

SEQ ID NO: 1289 - DNA, 20 bp, Glycine max
  primer
ccttaaacgttgcttcccac

SEQ ID NO: 1290 - DNA, 19 bp, Glycine max
  primer
cttgcaaatgctggggttt

SEQ ID NO: 1291 - DNA, 20 bp, Glycine max
  primer
tcatgcacccaacattcatc

SEQ ID NO: 1292 - DNA, 20 bp, Glycine max
  primer
gacactgcactctccatcca

SEQ ID NO: 1293 - DNA, 20 bp, Glycine max
  primer
gacccatcacgaaaagagga

SEQ ID NO: 1294 - DNA, 20 bp, Glycine max
  primer
aaagctgtttgtgcagagca

SEQ ID NO: 1295 - DNA, 20 bp, Glycine max
  primer
gccatgtagcacatgactcg

SEQ ID NO: 1296 - DNA, 20 bp, Glycine max
  primer
cccgtttattctgggaaaca

SEQ ID NO: 1297 - DNA, 20 bp, Glycine max
  primer
ttcccaacacaacacgtgaa

SEQ ID NO: 1298 - DNA, 20 bp, Glycine max
  primer
tgtttcccagttttgaaccc

SEQ ID NO: 1299 - DNA, 20 bp, Glycine max
  primer
tggctttgtttttcggctac

SEQ ID NO: 1300 - DNA, 20 bp, Glycine max
  primer
tgatgagcagcagcattttt

SEQ ID NO: 1301 - DNA, 21 bp, Glycine max
  primer
gaggaaacatttcttcggatg

SEQ ID NO: 1302 - DNA, 20 bp, Glycine max
  primer
cgggtaatcgtcctgcaata

SEQ ID NO: 1303 - DNA, 20 bp, Glycine max
  primer
caaaaagccttggactgagc

SEQ ID NO: 1304 - DNA, 20 bp, Glycine max
  primer
ggcagcagtttggctatttc

SEQ ID NO: 1305 - DNA, 20 bp, Glycine max
  primer
ccagagcacaaagatggtga

SEQ ID NO: 1306 - DNA, 20 bp, Glycine max
  primer
tggccatgtttttggatgta

SEQ ID NO: 1307 - DNA, 21 bp, Glycine max
  primer
tcatcaattgcagcttctgac

SEQ ID NO: 1308 - DNA, 20 bp, Glycine max
  primer
tgatttttcatcagtcacgg

SEQ ID NO: 1309 - DNA, 20 bp, Glycine max
  primer
caagctttcaaccccatgat

SEQ ID NO: 1310 - DNA, 20 bp, Glycine max
  primer
gaaatgggctcaacctgttc

SEQ ID NO: 1311 - DNA, 20 bp, Glycine max
  primer
ttttgggttcgaatttgagg

SEQ ID NO: 1312 - DNA, 20 bp, Glycine max
  primer
acaactatgcctccaccagc

SEQ ID NO: 1313 - DNA, 20 bp, Glycine max
  primer
cactcagtctcgtgcttcca

SEQ ID NO: 1314 - DNA, 20 bp, Glycine max
  primer
ccttctgaaatcaacacgca

SEQ ID NO: 1315 - DNA, 20 bp, Glycine max
  primer
ttagaatccaatccctcccc

SEQ ID NO: 1316 - DNA, 20 bp, Glycine max
  primer
gttggcacccaaacgataac

SEQ ID NO: 1317 - DNA, 20 bp, Glycine max
  primer
atcaacggcagaagcagagt

SEQ ID NO: 1318 - DNA, 20 bp, Glycine max
  primer
ggatttggttttggggttct

SEQ ID NO: 1319 - DNA, 20 bp, Glycine max
  primer
cgctgccatcactttctaca

SEQ ID NO: 1320 - DNA, 20 bp, Glycine max
  primer
agaaactggtgctgccaact

SEQ ID NO: 1321 - DNA, 20 bp, Glycine max
  primer
tctgggatgatgatgttgga

SEQ ID NO: 1322 - DNA, 20 bp, Glycine max
  primer
ctttggtgttgttgccaatg

SEQ ID NO: 1323 - DNA, 20 bp, Glycine max
  primer
ttggttgcatccattgctaa

SEQ ID NO: 1324 - DNA, 20 bp, Glycine max
  primer
atgaccaattgggtggttgt

SEQ ID NO: 1325 - DNA, 20 bp, Glycine max
  primer
catgtgtaattccactggcg

SEQ ID NO: 1326 - DNA, 20 bp, Glycine max
  primer
tggggaggagagcaactcta

SEQ ID NO: 1327 - DNA, 20 bp, Glycine max
  primer
ttgccagcctctatcattcc

SEQ ID NO: 1328 - DNA, 20 bp, Glycine max
  primer
tgatgggtgtgaatggaaaa

SEQ ID NO: 1329 - DNA, 20 bp, Glycine max
  primer
gatcgattggaagagcttgg

SEQ ID NO: 1330 - DNA, 20 bp, Glycine max
  primer
gatcatggttatggggcatc

SEQ ID NO: 1331 - DNA, 20 bp, Glycine max
  primer
agaatcgatacatgcgggtt

SEQ ID NO: 1332 - DNA, 20 bp, Glycine max
  primer
gcaactcacggatcctcgta

SEQ ID NO: 1333 - DNA, 20 bp, Glycine max
  primer
tattatgactcgcatgggca

SEQ ID NO: 1334 - DNA, 20 bp, Glycine max
  primer
tgaatggtggaagtgtccaa

SEQ ID NO: 1335 - DNA, 20 bp, Glycine max
  primer
agaaattgaaccggctgatg

SEQ ID NO: 1336 - DNA, 20 bp, Glycine max
  primer
cccaaagaatccccacctat

SEQ ID NO: 1337 - DNA, 20 bp, Glycine max
  primer
cctacaacaacggtgcattg

SEQ ID NO: 1338 - DNA, 20 bp, Glycine max
  primer
ccctccgttgctgttaccta

SEQ ID NO: 1339 - DNA, 20 bp, Glycine max
  primer
aaaggttcgagatgcgctta

SEQ ID NO: 1340 - DNA, 21 bp, Glycine max
  primer
tgattgatgagcattcagcag

SEQ ID NO: 1341 - DNA, 20 bp, Glycine max
  primer
acacacaacacagaacgacg

SEQ ID NO: 1342 - DNA, 21 bp, Glycine max
  primer
ctcgggaataatcagatgtcg

SEQ ID NO: 1343 - DNA, 20 bp, Glycine max
  primer
tctcccacatggaacacaaa

SEQ ID NO: 1344 - DNA, 20 bp, Glycine max
  primer
tggaaaccaacgggaataga

SEQ ID NO: 1345 - DNA, 20 bp, Glycine max
  primer
agaaggaaaagtggcaccct

SEQ ID NO: 1346 - DNA, 20 bp, Glycine max
  primer
tttgtctctttggggactcg

SEQ ID NO: 1347 - DNA, 20 bp, Glycine max
  primer
gcttggtgacccttttaggc

SEQ ID NO: 1348 - DNA, 22 bp, Glycine max
  primer
tgggttattgcttagacccttt

SEQ ID NO: 1349 - DNA, 20 bp, Glycine max
  primer
agctaaggggctgtctaggg

SEQ ID NO: 1350 - DNA, 20 bp, Glycine max
  primer
gatgctgctcaggaagaagg

SEQ ID NO: 1351 - DNA, 20 bp, Glycine max
  primer
tgcttcagggtattggaagg

SEQ ID NO: 1352 - DNA, 20 bp, Glycine max
  primer
ttcacaccaacgctctcttg

SEQ ID NO: 1353 - DNA, 20 bp, Glycine max
  primer
aatcagcggttaatgcttgg

SEQ ID NO: 1354 - DNA, 20 bp, Glycine max
  primer
tttggtgtgctcagcttctg

SEQ ID NO: 1355 - DNA, 20 bp, Glycine max
  primer
aagttgccaattgggttcag

SEQ ID NO: 1356 - DNA, 20 bp, Glycine max
  primer
gttgagcaaacgccttcttc

SEQ ID NO: 1357 - DNA, 20 bp, Glycine max
  primer
aggacgcgtttcgttttcta

SEQ ID NO: 1358 - DNA, 20 bp, Glycine max
  primer
gaagccagaaagcgatcaac

SEQ ID NO: 1359 - DNA, 20 bp, Glycine max
  primer
aacaagacgagaaggaggca

SEQ ID NO: 1360 - DNA, 23 bp, Glycine max
  primer
cgtactctgtaatttggttcagg

SEQ ID NO: 1361 - DNA, 20 bp, Glycine max
  primer
ccgagctttgaatcgaatgt

SEQ ID NO: 1362 - DNA, 20 bp, Glycine max
  primer
aatggaagtccctttctgcc

SEQ ID NO: 1363 - DNA, 20 bp, Glycine max
  primer
gcacttcagacatcaggggt

SEQ ID NO: 1364 - DNA, 20 bp, Glycine max
  primer
gcatagcatgcacgttgttt

SEQ ID NO: 1365 - DNA, 20 bp, Glycine max
  primer
tcttggagttcctcgtgtca

SEQ ID NO: 1366 - DNA, 22 bp, Glycine max
  primer
cgaccttttacaattcttgcag

SEQ ID NO: 1367 - DNA, 22 bp, Glycine max
  primer
ggaaaaaccatactttgtcagc

SEQ ID NO: 1368 - DNA, 20 bp, Glycine max
  primer
aatttgtccctcctgcatca

SEQ ID NO: 1369 - DNA, 20 bp, Glycine max
  primer
tttatgcctgaggtgacgtg

SEQ ID NO: 1370 - DNA, 20 bp, Glycine max
  primer
acacatcctcgtgctgattg

SEQ ID NO: 1371 - DNA, 20 bp, Glycine max
  primer
acgcaagggagagctgataa

SEQ ID NO: 1372 - DNA, 20 bp, Glycine max
  primer
ttccttcccggacacaagta

SEQ ID NO: 1373 - DNA, 20 bp, Glycine max
  primer
aatcgaaggtcttgctgtgg

SEQ ID NO: 1374 - DNA, 21 bp, Glycine max
  primer
agtaaaggccctgaacagttt

SEQ ID NO: 1375 - DNA, 20 bp, Glycine max
  primer
tagctttgtaatggggcgtg

SEQ ID NO: 1376 - DNA, 20 bp, Glycine max
  primer
ccgtgaacttgcacgattat

SEQ ID NO: 1377 - DNA, 20 bp, Glycine max
  primer
gcgatatctctgctccaagg

SEQ ID NO: 1378 - DNA, 20 bp, Glycine max
  primer
acagtcagggccaaaacaac

SEQ ID NO: 1379 - DNA, 20 bp, Glycine max
  primer
gatgctcaagaaggacgagg

SEQ ID NO: 1380 - DNA, 20 bp, Glycine max
  primer
gttgtacgcatactggggct

SEQ ID NO: 1381 - DNA, 20 bp, Glycine max
  primer
ccggtgtttatccactgctt

SEQ ID NO: 1382 - DNA, 20 bp, Glycine max
  primer
gcaagtgcatcatttcatgg

SEQ ID NO: 1383 - DNA, 20 bp, Glycine max
  primer
agggggagaatgacgagact

SEQ ID NO: 1384 - DNA, 20 bp, Glycine max
  primer
tgcactttttccagttgcac

SEQ ID NO: 1385 - DNA, 20 bp, Glycine max
  primer
caagcccatgtccctaaaag

SEQ ID NO: 1386 - DNA, 20 bp, Glycine max
  primer
aatggaagcaatcaacgacc

SEQ ID NO: 1387 - DNA, 20 bp, Glycine max
  primer
taagccgccagtgaaatcat

SEQ ID NO: 1388 - DNA, 20 bp, Glycine max
  primer
gcacttttggcctgttcagt

SEQ ID NO: 1389 - DNA, 20 bp, Glycine max
  primer
acatgccagtgagtgcagat

SEQ ID NO: 1390 - DNA, 20 bp, Glycine max
  primer
gtgttggttcagtcccatgt

SEQ ID NO: 1391 - DNA, 20 bp, Glycine max
  primer
ctgcaagtacggggttcact

SEQ ID NO: 1392 - DNA, 20 bp, Glycine max
  primer
ttctccaggggagattcctt

SEQ ID NO: 1393 - DNA, 20 bp, Glycine max
  primer
tatcaagatgccccaagagc

SEQ ID NO: 1394 - DNA, 20 bp, Glycine max
  primer
gcaaaacatggacattgacg

SEQ ID NO: 1395 - DNA, 20 bp, Glycine max
  primer
catggcaattgaaacacctg

SEQ ID NO: 1396 - DNA, 20 bp, Glycine max
  primer
gtggaagaaatgacggagga

SEQ ID NO: 1397 - DNA, 20 bp, Glycine max
  primer
tgcgataagcatcaagaacg

SEQ ID NO: 1398 - DNA, 20 bp, Glycine max
  primer
ccgataagcgtgggaaaata

SEQ ID NO: 1399 - DNA, 20 bp, Glycine max
  primer
gagtgggcaaatcccaaata

SEQ ID NO: 1400 - DNA, 20 bp, Glycine max
  primer
tgcttgggctcctcatagtt

SEQ ID NO: 1401 - DNA, 20 bp, Glycine max
  primer
ggcagaaacagttgcctcat

SEQ ID NO: 1402 - DNA, 20 bp, Glycine max
  primer
agcaacaatagatccgtggg

SEQ ID NO: 1403 - DNA, 20 bp, Glycine max
  primer
gttcttccgtgttttcggac

SEQ ID NO: 1404 - DNA, 20 bp, Glycine max
  primer
cttggctgccacatacagaa

SEQ ID NO: 1405 - DNA, 20 bp, Glycine max
  primer
tgggggaatccatgttattg

SEQ ID NO: 1406 - DNA, 20 bp, Glycine max
  primer
acaccttgttgattgcgttg

SEQ ID NO: 1407 - DNA, 21 bp, Glycine max
  primer
ccaccttgagttaacacctcg

SEQ ID NO: 1408 - DNA, 20 bp, Glycine max
  primer
gcattatggtgctgttccct

SEQ ID NO: 1409 - DNA, 20 bp, Glycine max
  primer
attaattcgcttcgtggtgc

SEQ ID NO: 1410 - DNA, 20 bp, Glycine max
  primer
ccaaagtgccgaggtattgt

SEQ ID NO: 1411 - DNA, 20 bp, Glycine max
  primer
tccaagctgtatctggcctt

SEQ ID NO: 1412 - DNA, 20 bp, Glycine max
  primer
ccgtggttcttttggttgat

SEQ ID NO: 1413 - DNA, 20 bp, Glycine max
  primer
agtccacccacaggtttcac

SEQ ID NO: 1414 - DNA, 20 bp, Glycine max
  primer
atgcctttacattcgcatcc

SEQ ID NO: 1415 - DNA, 20 bp, Glycine max
  primer
ggcaaattcaattcttggga

SEQ ID NO: 1416 - DNA, 20 bp, Glycine max
  primer
taaaactgaggggcctgatg

SEQ ID NO: 1417 - DNA, 20 bp, Glycine max
  primer
ctcaagccacttcatttggt

SEQ ID NO: 1418 - DNA, 21 bp, Glycine max
  primer
tttcccaagaaactaccttcc

SEQ ID NO: 1419 - DNA, 20 bp, Glycine max
  primer
agaattcatcccctccttga

SEQ ID NO: 1420 - DNA, 22 bp, Glycine max
  primer
tgatgatgatgatgatatgcac

SEQ ID NO: 1421 - DNA, 20 bp, Glycine max
  primer
gtgcaggatgtctacgggac

SEQ ID NO: 1422 - DNA, 20 bp, Glycine max
  primer
ggctttctcagctttgggta

SEQ ID NO: 1423 - DNA, 20 bp, Glycine max
  primer
tggttcatggctttgtgaga

SEQ ID NO: 1424 - DNA, 20 bp, Glycine max
  primer
tgacccaaacggagaagaag

SEQ ID NO: 1425 - DNA, 20 bp, Glycine max
  primer
caccttgcagaatatccggt

SEQ ID NO: 1426 - DNA, 20 bp, Glycine max
  primer
caaaagcttgggaaaccaaa

SEQ ID NO: 1427 - DNA, 20 bp, Glycine max
  primer
aaagtggcggttgttgaaag

SEQ ID NO: 1428 - DNA, 20 bp, Glycine max
  primer
aaaggtggagcaatgcaatc

SEQ ID NO: 1429 - DNA, 20 bp, Glycine max
  primer
agcaatggtggagccataag

SEQ ID NO: 1430 - DNA, 20 bp, Glycine max
  primer
ccggacagtcttcccagtag

SEQ ID NO: 1431 - DNA, 20 bp, Glycine max
  primer
tggagtgacgacgatgagtc

SEQ ID NO: 1432 - DNA, 20 bp, Glycine max
  primer
atgctttggagttttcccct

SEQ ID NO: 1433 - DNA, 20 bp, Glycine max
  primer
ccagcgctgatttgatgtta

SEQ ID NO: 1434 - DNA, 20 bp, Glycine max
  primer
ccagcagaaagctccaaaac

SEQ ID NO: 1435 - DNA, 20 bp, Glycine max
  primer
ctctcacccaaaatccctca

SEQ ID NO: 1436 - DNA, 20 bp, Glycine max
  primer
atggctaatggatccccttt

SEQ ID NO: 1437 - DNA, 20 bp, Glycine max
  primer
gatgacaaggtcccacgaat

SEQ ID NO: 1438 - DNA, 20 bp, Glycine max
  primer
gccaagcaacctcttctttg

SEQ ID NO: 1439 - DNA, 20 bp, Glycine max
  primer
ggagaagtgaggtgtgaggc

SEQ ID NO: 1440 - DNA, 20 bp, Glycine max
  primer
aatttgtgggctccactgtc

SEQ ID NO: 1441 - DNA, 20 bp, Glycine max
  primer
gttcagtgttgcagccatgt

SEQ ID NO: 1442 - DNA, 21 bp, Glycine max
  primer
aacctacccaacgtagcaaaa

SEQ ID NO: 1443 - DNA, 20 bp, Glycine max
  primer
tgaagatccccaatcccata

SEQ ID NO: 1444 - DNA, 20 bp, Glycine max
  primer
ctttggtggctcggatctaa

SEQ ID NO: 1445 - DNA, 20 bp, Glycine max
  primer
atctggctttgccaatttgt

SEQ ID NO: 1446 - DNA, 20 bp, Glycine max
  primer
gtcaggcatttcctgcttct

SEQ ID NO: 1447 - DNA, 20 bp, Glycine max
  primer
ttatccgagtccattttggg

SEQ ID NO: 1448 - DNA, 20 bp, Glycine max
  primer
gccattcagaacacgaggtt

SEQ ID NO: 1449 - DNA, 20 bp, Glycine max
  primer
taggccctttcaaccacaac

SEQ ID NO: 1450 - DNA, 20 bp, Glycine max
  primer
atccagctgtccgaacttgt

SEQ ID NO: 1451 - DNA, 20 bp, Glycine max
  primer
gagaaccaaacgctggatgt

SEQ ID NO: 1452 - DNA, 20 bp, Glycine max
  primer
gcgagtccttttcaccactc

SEQ ID NO: 1453 - DNA, 20 bp, Glycine max
  primer
acattatggcttgtgccgat

SEQ ID NO: 1454 - DNA, 20 bp, Glycine max
  primer
actgtgtcatgattcgcagc

SEQ ID NO: 1455 - DNA, 20 bp, Glycine max
  primer
agaccaagaccagaacgacg

SEQ ID NO: 1456 - DNA, 20 bp, Glycine max
  primer
gctccaaacaaagaaaccca

SEQ ID NO: 1457 - DNA, 20 bp, Glycine max
  primer
ctgcagggtagagttggagc

SEQ ID NO: 1458 - DNA, 20 bp, Glycine max
  primer
gtgcatcttcatcaacaccg

SEQ ID NO: 1459 - DNA, 20 bp, Glycine max
  primer
aggaaccccctgagagctac

SEQ ID NO: 1460 - DNA, 21 bp, Glycine max
  primer
gcaaagaagaacgacagagga

SEQ ID NO: 1461 - DNA, 20 bp, Glycine max
  primer
acgcctatgaacgtgaaacc

SEQ ID NO: 1462 - DNA, 20 bp, Glycine max
  primer
gcattcggtgggaattagaa

SEQ ID NO: 1463 - DNA, 20 bp, Glycine max
  primer
gggaaaacctcatgagtcca

SEQ ID NO: 1464 - DNA, 20 bp, Glycine max
  primer
gtccggtaggctcgatacaa

SEQ ID NO: 1465 - DNA, 20 bp, Glycine max
  primer
ggagttgttgtgagcgtgtg

SEQ ID NO: 1466 - DNA, 22 bp, Glycine max
  primer
tatttgatcgtagatccagcac

SEQ ID NO: 1467 - DNA, 20 bp, Glycine max
  primer
tggtttgtgcaaatatcccc

SEQ ID NO: 1468 - DNA, 20 bp, Glycine max
  primer
caattgtgagaaagagcgca

SEQ ID NO: 1469 - DNA, 20 bp, Glycine max
  primer
agaagttgtgcaaaatgggg

SEQ ID NO: 1470 - DNA, 20 bp, Glycine max
  primer
ttgtgcaagatcccctaacc

SEQ ID NO: 1471 - DNA, 20 bp, Glycine max
  primer
gagagagggaagcccgttag

SEQ ID NO: 1472 - DNA, 20 bp, Glycine max
  primer
tccaccaataacaccaacca

SEQ ID NO: 1473 - DNA, 20 bp, Glycine max
  primer
tttaggacagttgcttgggc

SEQ ID NO: 1474 - DNA, 20 bp, Glycine max
  primer
gagagtgtcggggatgtgtt

SEQ ID NO: 1475 - DNA, 20 bp, Glycine max
  primer
cccatggagcaaatacacct

SEQ ID NO: 1476 - DNA, 20 bp, Glycine max
  primer
agcaagcaaaagtttccagg

SEQ ID NO: 1477 - DNA, 20 bp, Glycine max
  primer
gtccgattggagaatcatgc

SEQ ID NO: 1478 - DNA, 20 bp, Glycine max
  primer
gaatctcaaattcggtccca

SEQ ID NO: 1479 - DNA, 20 bp, Glycine max
  primer
tatggggctataccgctacg

SEQ ID NO: 1480 - DNA, 20 bp, Glycine max
  primer
cgccttctatacccactgga

SEQ ID NO: 1481 - DNA, 20 bp, Glycine max
  primer
ctcttcacggacttcttgcc

SEQ ID NO: 1482 - DNA, 20 bp, Glycine max
  primer
aaggatcgcgtttagaacca

SEQ ID NO: 1483 - DNA, 20 bp, Glycine max
  primer
cgcgtccgataacaataaca

SEQ ID NO: 1484 - DNA, 20 bp, Glycine max
  primer
agagaattgccgatggtgat

SEQ ID NO: 1485 - DNA, 21 bp, Glycine max
  primer
cccagatgcttacacaaaagc

SEQ ID NO: 1486 - DNA, 20 bp, Glycine max
  primer
cagaatttgagtgcgcttga

SEQ ID NO: 1487 - DNA, 20 bp, Glycine max
  primer
aggcaaaaggggataaatgc

SEQ ID NO: 1488 - DNA, 20 bp, Glycine max
  primer
gcttgtttcaaatggctcgt

SEQ ID NO: 1489 - DNA, 20 bp, Glycine max
  primer
aggcactttgttttcccttg

SEQ ID NO: 1490 - DNA, 20 bp, Glycine max
  primer
tgcatgtttactgcagcgat

SEQ ID NO: 1491 - DNA, 21 bp, Glycine max
  primer
aaactggagctttgacaccaa

SEQ ID NO: 1492 - DNA, 20 bp, Glycine max
  primer
atatgttcatccctggctgc

SEQ ID NO: 1493 - DNA, 20 bp, Glycine max
  primer
aaagaagccaacaggcagaa

SEQ ID NO: 1494 - DNA, 20 bp, Glycine max
  primer
ccttccgatgcagaaatcat

SEQ ID NO: 1495 - DNA, 20 bp, Glycine max
  primer
aagttgtatggttgggcctg

SEQ ID NO: 1496 - DNA, 20 bp, Glycine max
  primer
atccccgcctcatactatcc

SEQ ID NO: 1497 - DNA, 20 bp, Glycine max
  primer
ttgatgtggaaaggggacac

SEQ ID NO: 1498 - DNA, 20 bp, Glycine max
  primer
cgttggcaaagttatcggtt

SEQ ID NO: 1499 - DNA, 20 bp, Glycine max
  primer
gtgtgttgaggggttttggt

SEQ ID NO: 1500 - DNA, 20 bp, Glycine max
  primer
ctctgcttctgcttgaaccc

SEQ ID NO: 1501 - DNA, 20 bp, Glycine max
  primer
atgtggttgttgttggttgg

SEQ ID NO: 1502 - DNA, 23 bp, Glycine max
  primer
cacttgacagctgaattccagta

SEQ ID NO: 1503 - DNA, 19 bp, Glycine max
  primer
ggccgtgttaaaacgtgtg

SEQ ID NO: 1504 - DNA, 20 bp, Glycine max
  primer
ggcttttgctttagccagtg

SEQ ID NO: 1505 - DNA, 20 bp, Glycine max
  primer
gtttacgcaaacaccgacct

SEQ ID NO: 1506 - DNA, 20 bp, Glycine max
  primer
attggatgcagagggttttg

SEQ ID NO: 1507 - DNA, 20 bp, Glycine max
  primer
cgacaagaagaatgcgaaca

SEQ ID NO: 1508 - DNA, 20 bp, Glycine max
  primer
ctgagactcactggccttcc

SEQ ID NO: 1509 - DNA, 20 bp, Glycine max
  primer
ccaagatcaagtgcaacacc

SEQ ID NO: 1510 - DNA, 20 bp, Glycine max
  primer
ggacccatgtgaaattgacc

SEQ ID NO: 1511 - DNA, 20 bp, Glycine max
  primer
gcactgtttttccatcgtca

SEQ ID NO: 1512 - DNA, 20 bp, Glycine max
  primer
ctcgtgaccattgtggtttg

SEQ ID NO: 1513 - DNA, 20 bp, Glycine max
  primer
tgctgggtgatattggtgaa

SEQ ID NO: 1514 - DNA, 20 bp, Glycine max
  primer
gtctctgctggcaccattct

SEQ ID NO: 1515 - DNA, 20 bp, Glycine max
  primer
atggggagcatatgcagtgt

SEQ ID NO: 1516 - DNA, 20 bp, Glycine max
  primer
tcgaccaagtagggtcttga

SEQ ID NO: 1517 - DNA, 20 bp, Glycine max
  primer
caaggctgttccaacacaaa

SEQ ID NO: 1518 - DNA, 20 bp, Glycine max
  primer
tagccatcatcaagacgcag

SEQ ID NO: 1519 - DNA, 20 bp, Glycine max
  primer
atggccaattggagtattgc

SEQ ID NO: 1520 - DNA, 20 bp, Glycine max
  primer
ggacaaccagtcaagggaaa

SEQ ID NO: 1521 - DNA, 20 bp, Glycine max
  primer
cgtcggattagaacccttga

SEQ ID NO: 1522 - DNA, 20 bp, Glycine max
  primer
gctttttcacgaaagcaacc

SEQ ID NO: 1523 - DNA, 20 bp, Glycine max
  primer
atcacaatgcttggagaccc

SEQ ID NO: 1524 - DNA, 20 bp, Glycine max
  primer
tgtgcttgtctgagtcctgg

SEQ ID NO: 1525 - DNA, 20 bp, Glycine max
  primer
tttttcctcgcagttatgcc

SEQ ID NO: 1526 - DNA, 21 bp, Glycine max
  primer
tccaaagactaagagggggaa

SEQ ID NO: 1527 - DNA, 20 bp, Glycine max
  primer
tgccatgcgtattttctgag

SEQ ID NO: 1528 - DNA, 20 bp, Glycine max
  primer
ggccgcaagctttttaatct

SEQ ID NO: 1529 - DNA, 20 bp, Glycine max
  primer
acaagcgaaggaaggagtga

SEQ ID NO: 1530 - DNA, 20 bp, Glycine max
  primer
gtccgtcccttgctattcaa

SEQ ID NO: 1531 - DNA, 20 bp, Glycine max
  primer
gtccctttgcagtggtgact

SEQ ID NO: 1532 - DNA, 20 bp, Glycine max
  primer
tcaagatctgccaccaaatg

SEQ ID NO: 1533 - DNA, 20 bp, Glycine max
  primer
ctctgctggtggaagttggt

SEQ ID NO: 1534 - DNA, 20 bp, Glycine max
  primer
gatcccgaaatcatccgtaa

SEQ ID NO: 1535 - DNA, 20 bp, Glycine max
  primer
tatttaaaggtggtcgccct

SEQ ID NO: 1536 - DNA, 20 bp, Glycine max
  primer
atgacagcgatgaagaggct

SEQ ID NO: 1537 - DNA, 20 bp, Glycine max
  primer
actgcattcattccggtttc

SEQ ID NO: 1538 - DNA, 20 bp, Glycine max
  primer
ggaagaaatccttcgggttc

SEQ ID NO: 1539 - DNA, 20 bp, Glycine max
  primer
ttttggacggctaagtgtca

SEQ ID NO: 1540 - DNA, 20 bp, Glycine max
  primer
tcagataaggtgcgcagttg

SEQ ID NO: 1541 - DNA, 20 bp, Glycine max
  primer
ggattcagtcacagcagcaa

SEQ ID NO: 1542 - DNA, 20 bp, Glycine max
  primer
acaccgagagacgaccagac

SEQ ID NO: 1543 - DNA, 20 bp, Glycine max
  primer
cagtgggagaaggagcgata

SEQ ID NO: 1544 - DNA, 20 bp, Glycine max
  primer
ccgaaatatcggaagggatt

SEQ ID NO: 1545 - DNA, 20 bp, Glycine max
  primer
gcctcttgatgacactgcaa

SEQ ID NO: 1546 - DNA, 20 bp, Glycine max
  primer
ttcaatgcactctccactgc

SEQ ID NO: 1547 - DNA, 20 bp, Glycine max
  primer
ttttcgaacagcctccctaa

SEQ ID NO: 1548 - DNA, 20 bp, Glycine max
  primer
atgcggagtgatggttatgt

SEQ ID NO: 1549 - DNA, 20 bp, Glycine max
  primer
catctacgggtactggcgat

SEQ ID NO: 1550 - DNA, 20 bp, Glycine max
  primer
tccggaaaccagaacttgac

SEQ ID NO: 1551 - DNA, 20 bp, Glycine max
  primer
tgcttgagcaaggttttgtg

SEQ ID NO: 1552 - DNA, 20 bp, Glycine max
  primer
aacatggctgacgtatgggt

SEQ ID NO: 1553 - DNA, 20 bp, Glycine max
  primer
gcaactcgtgaaaggtaggc

SEQ ID NO: 1554 - DNA, 20 bp, Glycine max
  primer
tttcatccggcacagtatca

SEQ ID NO: 1555 - DNA, 20 bp, Glycine max
  primer
tccattgaggaattgcatga

SEQ ID NO: 1556 - DNA, 20 bp, Glycine max
  primer
gcgttgaaacagatttgggt

SEQ ID NO: 1557 - DNA, 20 bp, Glycine max
  primer
cgttcatcaatggcagaaga

SEQ ID NO: 1558 - DNA, 20 bp, Glycine max
  primer
aaggagcattgctgcatttt

SEQ ID NO: 1559 - DNA, 20 bp, Glycine max
  primer
ccatggatgctgaggaactt

SEQ ID NO: 1560 - DNA, 20 bp, Glycine max
  primer
ctgccacttcatcctttggt

SEQ ID NO: 1561 - DNA, 20 bp, Glycine max
  primer
acaatcaaccgaggctcaac

SEQ ID NO: 1562 - DNA, 20 bp, Glycine max
  primer
cgaatcatcgtcctcatcct

SEQ ID NO: 1563 - DNA, 20 bp, Glycine max
  primer
cccaggtatggtccttctca

SEQ ID NO: 1564 - DNA, 20 bp, Glycine max
  primer
cttctaccccatggcaagag

SEQ ID NO: 1565 - DNA, 20 bp, Glycine max
  primer
ccgtgctgttgtggaatatg

SEQ ID NO: 1566 - DNA, 20 bp, Glycine max
  primer
accaggacacctgactccag

SEQ ID NO: 1567 - DNA, 20 bp, Glycine max
  primer
ccggtctttctaggaggagg

SEQ ID NO: 1568 - DNA, 20 bp, Glycine max
  primer
tccaggatgaagcaaagacc

SEQ ID NO: 1569 - DNA, 20 bp, Glycine max
  primer
ggccgtagttgactgtaggg

SEQ ID NO: 1570 - DNA, 20 bp, Glycine max
  primer
agttgaatcccccaacgact

SEQ ID NO: 1571 - DNA, 20 bp, Glycine max
  primer
gtgtccaaaaatgggcaatc

SEQ ID NO: 1572 - DNA, 20 bp, Glycine max
  primer
tgacgaccaatgaggtgtgt

SEQ ID NO: 1573 - DNA, 20 bp, Glycine max
  primer
cacaaaaacctcaactgcga

SEQ ID NO: 1574 - DNA, 20 bp, Glycine max
  primer
aataaaaggtgcatgtggca

SEQ ID NO: 1575 - DNA, 20 bp, Glycine max
  primer
tgcattttaccccctttgaa

SEQ ID NO: 1576 - DNA, 20 bp, Glycine max
  primer
agggttttggggattttgtc

SEQ ID NO: 1577 - DNA, 20 bp, Glycine max
  primer
cggaaaccctacggtagaca

SEQ ID NO: 1578 - DNA, 20 bp, Glycine max
  primer
cagtgcttcgggaagatagg

SEQ ID NO: 1579 - DNA, 22 bp, Glycine max
  primer
ggttgactatttccacctacct

SEQ ID NO: 1580 - DNA, 21 bp, Glycine max
  primer
tgctgtctttttgtctcagtg

SEQ ID NO: 1581 - DNA, 20 bp, Glycine max
  primer
aaaaagacgaccacagcgac

SEQ ID NO: 1582 - DNA, 20 bp, Glycine max
  primer
atcatcgtcgtcgtcatcaa

SEQ ID NO: 1583 - DNA, 21 bp, Glycine max
  primer
catcaattcaagagaatgggg

SEQ ID NO: 1584 - DNA, 22 bp, Glycine max
  primer
cttctgaagaatgcctaattgc

SEQ ID NO: 1585 - DNA, 20 bp, Glycine max
  primer
agcagcaggacagaacaggt

SEQ ID NO: 1586 - DNA, 20 bp, Glycine max
  primer
agcagccctacatggacatc

SEQ ID NO: 1587 - DNA, 20 bp, Glycine max
  primer
cgaaaggatgaaactctcgc

SEQ ID NO: 1588 - DNA, 20 bp, Glycine max
  primer
gccaaatactttccgatcca

SEQ ID NO: 1589 - DNA, 20 bp, Glycine max
  primer
cgaaacggaaccaaagaaga

SEQ ID NO: 1590 - DNA, 20 bp, Glycine max
  primer
cttcaacctcgggtgattgt

SEQ ID NO: 1591 - DNA, 20 bp, Glycine max
  primer
gaggaatcgacgttggtgat

SEQ ID NO: 1592 - DNA, 20 bp, Glycine max
  primer
ccgtctctttccatctgctc

SEQ ID NO: 1593 - DNA, 20 bp, Glycine max
  primer
taccctttccctgctcctct

SEQ ID NO: 1594 - DNA, 20 bp, Glycine max
  primer
cgattgacaactcaaccgag

SEQ ID NO: 1595 - DNA, 20 bp, Glycine max
  primer
tgatggtattgctgctccag

SEQ ID NO: 1596 - DNA, 20 bp, Glycine max
  primer
tgctgcagatcctgtttttg

SEQ ID NO: 1597 - DNA, 20 bp, Glycine max
  primer
tcaaaattgttggccagtga

SEQ ID NO: 1598 - DNA, 20 bp, Glycine max
  primer
tcttgtgcttgtttcatcgc

SEQ ID NO: 1599 - DNA, 20 bp, Glycine max
  primer
tgctcattgctacctcaacg

SEQ ID NO: 1600 - DNA, 20 bp, Glycine max
  primer
acggccatagatcaccaaag

SEQ ID NO: 1601 - DNA, 20 bp, Glycine max
  primer
ttcggaacagtttgtcgaag

SEQ ID NO: 1602 - DNA, 20 bp, Glycine max
  primer
gaccaatcacaacacatgcc

SEQ ID NO: 1603 - DNA, 20 bp, Glycine max
  primer
atatgatgactgccacgggt

SEQ ID NO: 1604 - DNA, 20 bp, Glycine max
  primer
tgctgtcctctcgaatgatg

SEQ ID NO: 1605 - DNA, 20 bp, Glycine max
  primer
ccaccttccccatgatacac

SEQ ID NO: 1606 - DNA, 20 bp, Glycine max
  primer
agaagacatgccctggactg

SEQ ID NO: 1607 - DNA, 20 bp, Glycine max
  primer
tacctatcaccgagaagcgg

SEQ ID NO: 1608 - DNA, 20 bp, Glycine max
  primer
atatgttcctggcgaaaacg

SEQ ID NO: 1609 - DNA, 20 bp, Glycine max
  primer
gtgagggagagacgaagacg

SEQ ID NO: 1610 - DNA, 20 bp, Glycine max
  primer
ctccattccctctcacgaaa

SEQ ID NO: 1611 - DNA, 20 bp, Glycine max
  primer
tcaagggcatggctataggt

SEQ ID NO: 1612 - DNA, 20 bp, Glycine max
  primer
ccagcacggttggattatct

SEQ ID NO: 1613 - DNA, 20 bp, Glycine max
  primer
atgaagctgcagccaaactt

SEQ ID NO: 1614 - DNA, 20 bp, Glycine max
  primer
cttcctcctcctccacaaga

SEQ ID NO: 1615 - DNA, 20 bp, Glycine max
  primer
accatcgtccgttcatcaat

SEQ ID NO: 1616 - DNA, 20 bp, Glycine max
  primer
tcctcagggagttgttttgg

SEQ ID NO: 1617 - DNA, 20 bp, Glycine max
  primer
gttgtgccagcatttcttga

SEQ ID NO: 1618 - DNA, 20 bp, Glycine max
  primer
aatttgagcccacaggtcag

SEQ ID NO: 1619 - DNA, 19 bp, Glycine max
  primer
attcggcacgagggtaatc

SEQ ID NO: 1620 - DNA, 23 bp, Glycine max
  primer
caacatcgtaaggaacattaggc

SEQ ID NO: 1621 - DNA, 20 bp, Glycine max
  primer
acagccagagcctcgttaaa

SEQ ID NO: 1622 - DNA, 20 bp, Glycine max
  primer
acgaagaggcagctgaagtc

SEQ ID NO: 1623 - DNA, 20 bp, Glycine max
  primer
ttacaagctgtggatgtgcc

SEQ ID NO: 1624 - DNA, 20 bp, Glycine max
  primer
tggatgaggtcttggtcctt

SEQ ID NO: 1625 - DNA, 19 bp, Glycine max
  primer
caaattggggtttccttcg

SEQ ID NO: 1626 - DNA, 20 bp, Glycine max
  primer
tttgcttgtcgagttcgatg

SEQ ID NO: 1627 - DNA, 20 bp, Glycine max
  primer
gtgatgagcgaactgtgcat

SEQ ID NO: 1628 - DNA, 20 bp, Glycine max
  primer
tgccagataaggctgcagta

SEQ ID NO: 1629 - DNA, 20 bp, Glycine max
  primer
gagctcagtcttcctcgtcg

SEQ ID NO: 1630 - DNA, 20 bp, Glycine max
  primer
agggttcgtgctttggtatg

SEQ ID NO: 1631 - DNA, 20 bp, Glycine max
  primer
agcgggtagagttcacgttg

SEQ ID NO: 1632 - DNA, 20 bp, Glycine max
  primer
tattgttgacgctcctccgt

SEQ ID NO: 1633 - DNA, 20 bp, Glycine max
  primer
tatggtggcatgaaaacagc

SEQ ID NO: 1634 - DNA, 21 bp, Glycine max
  primer
tgagcttttgaagagcaaagc

SEQ ID NO: 1635 - DNA, 20 bp, Glycine max
  primer
atatgcacccccagacaaaa

SEQ ID NO: 1636 - DNA, 20 bp, Glycine max
  primer
aaggccactggaatcatcag

SEQ ID NO: 1637 - DNA, 20 bp, Glycine max
  primer
gcacgtgttgttggtttttg

SEQ ID NO: 1638 - DNA, 20 bp, Glycine max
  primer
tatgactatgcatccctgcg

SEQ ID NO: 1639 - DNA, 20 bp, Glycine max
  primer
ccccaatgtaactttcccct

SEQ ID NO: 1640 - DNA, 20 bp, Glycine max
  primer
cacacttagctggaatggca

SEQ ID NO: 1641 - DNA, 20 bp, Glycine max
  primer
gattgggttgaagtgttggg

SEQ ID NO: 1642 - DNA, 20 bp, Glycine max
  primer
gcaagtttatgggcaaccag

SEQ ID NO: 1643 - DNA, 20 bp, Glycine max
  primer
cattggttcatatcccccac

SEQ ID NO: 1644 - DNA, 20 bp, Glycine max
  primer
cctagccgctactctccctt

SEQ ID NO: 1645 - DNA, 20 bp, Glycine max
  primer
gaatccgacataggccagaa

SEQ ID NO: 1646 - DNA, 20 bp, Glycine max
  primer
accccagattccaacctctc

SEQ ID NO: 1647 - DNA, 20 bp, Glycine max
  primer
ccattcccatggaaaacaac

SEQ ID NO: 1648 - DNA, 20 bp, Glycine max
  primer
ggcatttggctaggattgaa

SEQ ID NO: 1649 - DNA, 20 bp, Glycine max
  primer
gtggtctcagccttcaggac

SEQ ID NO: 1650 - DNA, 20 bp, Glycine max
  primer
taagtacaaaaccggcaccc

SEQ ID NO: 1651 - DNA, 20 bp, Glycine max
  primer
ctgaacagcggtaccaggat

SEQ ID NO: 1652 - DNA, 20 bp, Glycine max
  primer
gcagccaggttctctgattt

SEQ ID NO: 1653 - DNA, 22 bp, Glycine max
  primer
ctgcagactcagcaattgagat

SEQ ID NO: 1654 - DNA, 20 bp, Glycine max
  primer
agcctgattatgcccctttc

SEQ ID NO: 1655 - DNA, 20 bp, Glycine max
  primer
cgtgcatttattttcagggg

SEQ ID NO: 1656 - DNA, 20 bp, Glycine max
  primer
atgaggctggtgctgctact

SEQ ID NO: 1657 - DNA, 20 bp, Glycine max
  primer
ctggtacatacaacgtgccg

SEQ ID NO: 1658 - DNA, 20 bp, Glycine max
  primer
actcggaggatctgcttctg

SEQ ID NO: 1659 - DNA, 20 bp, Glycine max
  primer
gatggaagagaacgagcgac

SEQ ID NO: 1660 - DNA, 20 bp, Glycine max
  primer
ccgaagactgaccttcatcc

SEQ ID NO: 1661 - DNA, 20 bp, Glycine max
  primer
agtctgcaaggaagaaggca

SEQ ID NO: 1662 - DNA, 20 bp, Glycine max
  primer
ttgggctgatagcgtctttt

SEQ ID NO: 1663 - DNA, 20 bp, Glycine max
  primer
tcattcgttcatcagtggga

SEQ ID NO: 1664 - DNA, 20 bp, Glycine max
  primer
ttcatcactttctggcgttg

SEQ ID NO: 1665 - DNA, 20 bp, Glycine max
  primer
cgattgcaaggaagaggaag

SEQ ID NO: 1666 - DNA, 20 bp, Glycine max
  primer
ctattgcatttctcgacgca

SEQ ID NO: 1667 - DNA, 21 bp, Glycine max
  primer
agcagaggcaacagtatccaa

SEQ ID NO: 1668 - DNA, 20 bp, Glycine max
  primer
ctgctgtcaatggcacagat

SEQ ID NO: 1669 - DNA, 21 bp, Glycine max
  primer
tcttctggaagctatttcgca

SEQ ID NO: 1670 - DNA, 20 bp, Glycine max
  primer
attgattcgcaaaaggaagc

SEQ ID NO: 1671 - DNA, 20 bp, Glycine max
  primer
ggtccgcagaggattttgta

SEQ ID NO: 1672 - DNA, 20 bp, Glycine max
  primer
cccatgcttcaaagcagatt

SEQ ID NO: 1673 - DNA, 20 bp, Glycine max
  primer
agcctgacataaggtgtgcc

SEQ ID NO: 1674 - DNA, 20 bp, Glycine max
  primer
gacatgtattctcccggtgg

SEQ ID NO: 1675 - DNA, 21 bp, Glycine max
  primer
gggaagtgcaataatgaagca

SEQ ID NO: 1676 - DNA, 20 bp, Glycine max
  primer
tacgtagaagaaagggccga

SEQ ID NO: 1677 - DNA, 20 bp, Glycine max
  primer
ggtggctcttctgatgctct

SEQ ID NO: 1678 - DNA, 20 bp, Glycine max
  primer
ggtcgagatacaaagcctgc

SEQ ID NO: 1679 - DNA, 20 bp, Glycine max
  primer
ctcagccatgcaattcttca

SEQ ID NO: 1680 - DNA, 20 bp, Glycine max
  primer
attgttttgggaagcacagc

SEQ ID NO: 1681 - DNA, 20 bp, Glycine max
  primer
gcatacaacaagttcacccg

SEQ ID NO: 1682 - DNA, 20 bp, Glycine max
  primer
aagtccatttgccacagagg

SEQ ID NO: 1683 - DNA, 20 bp, Glycine max
  primer
attgttgaggcctgtatcgg

SEQ ID NO: 1684 - DNA, 20 bp, Glycine max
  primer
tgatggcagcttttaggtcc

SEQ ID NO: 1685 - DNA, 20 bp, Glycine max
  primer
gaagccggtgtcaaggacta

SEQ ID NO: 1686 - DNA, 20 bp, Glycine max
  primer
ggacactactctcggctgct

SEQ ID NO: 1687 - DNA, 20 bp, Glycine max
  primer
ggctgagctaactttgagcg

SEQ ID NO: 1688 - DNA, 22 bp, Glycine max
  primer
tgaagtcctgaatcagtagcca

SEQ ID NO: 1689 - DNA, 21 bp, Glycine max
  primer
aaaccattcactgtttgctgg

SEQ ID NO: 1690 - DNA, 20 bp, Glycine max
  primer
tggttaaccgaagggtttca

SEQ ID NO: 1691 - DNA, 20 bp, Glycine max
  primer
ttcccagccaaatttaagga

SEQ ID NO: 1692 - DNA, 20 bp, Glycine max
  primer
ggaatatgcaagaccctcca

SEQ ID NO: 1693 - DNA, 20 bp, Glycine max
  primer
acatatggatggtggccaat

SEQ ID NO: 1694 - DNA, 20 bp, Glycine max
  primer
tgcctcgatacaaagcactg

SEQ ID NO: 1695 - DNA, 20 bp, Glycine max
  primer
tttgaaccaagccaaaaacc

SEQ ID NO: 1696 - DNA, 20 bp, Glycine max
  primer
gtggacctaacaatgtgccc

SEQ ID NO: 1697 - DNA, 20 bp, Glycine max
  primer
gctggtgatggttgttgttg

SEQ ID NO: 1698 - DNA, 20 bp, Glycine max
  primer
tcgcctatagacggatccac

SEQ ID NO: 1699 - DNA, 20 bp, Glycine max
  primer
aaggttgaaaagctgcgaaa

SEQ ID NO: 1700 - DNA, 20 bp, Glycine max
  primer
gcactgcatctacacccaaa

SEQ ID NO: 1701 - DNA, 21 bp, Glycine max
  primer
tgagaagttccgaagatcgaa

SEQ ID NO: 1702 - DNA, 20 bp, Glycine max
  primer
gttgaagagcataggggcaa

SEQ ID NO: 1703 - DNA, 20 bp, Glycine max
  primer
ctgcttcctccgattctcac

SEQ ID NO: 1704 - DNA, 20 bp, Glycine max
  primer
cccaattgattccaaggaga

SEQ ID NO: 1705 - DNA, 20 bp, Glycine max
  primer
ctccagaaccagtagccagg

SEQ ID NO: 1706 - DNA, 20 bp, Glycine max
  primer
gctcgttgttgttgtggttg

SEQ ID NO: 1707 - DNA, 21 bp, Glycine max
  primer
ccccatattgttctttctccc

SEQ ID NO: 1708 - DNA, 20 bp, Glycine max
  primer
ttaagggcagaccaaagcag

SEQ ID NO: 1709 - DNA, 20 bp, Glycine max
  primer
accagcctttcccaactttt

SEQ ID NO: 1710 - DNA, 20 bp, Glycine max
  primer
tcagatgggttggtggtgta

SEQ ID NO: 1711 - DNA, 20 bp, Glycine max
  primer
tgctggctgaggtttctaca

SEQ ID NO: 1712 - DNA, 20 bp, Glycine max
  primer
aaggggctaaaccaaatcca

SEQ ID NO: 1713 - DNA, 20 bp, Glycine max
  primer
tgctgttgggtgaatgaaga

SEQ ID NO: 1714 - DNA, 20 bp, Glycine max
  primer
gttctcaaaatccattggcg

SEQ ID NO: 1715 - DNA, 20 bp, Glycine max
  primer
gtcggacttgtgtcccagtt

SEQ ID NO: 1716 - DNA, 19 bp, Glycine max
  primer
acacgaaaggtggagggtc

SEQ ID NO: 1717 - DNA, 20 bp, Glycine max
  primer
gaggttggcctccattgata

SEQ ID NO: 1718 - DNA, 20 bp, Glycine max
  primer
tctctctcttggtgttgggc

SEQ ID NO: 1719 - DNA, 20 bp, Glycine max
  primer
tgaccgggtttcaggagtaa

SEQ ID NO: 1720 - DNA, 20 bp, Glycine max
  primer
tctccatccatccctttctg

SEQ ID NO: 1721 - DNA, 20 bp, Glycine max
  primer
cggcactggtttccaagata

SEQ ID NO: 1722 - DNA, 20 bp, Glycine max
  primer
tcagcaacgttcgtcatttc

SEQ ID NO: 1723 - DNA, 20 bp, Glycine max
  primer
tcgacctctccaaatctgct

SEQ ID NO: 1724 - DNA, 20 bp, Glycine max
  primer
ttgtaagtggaaggggcatc

SEQ ID NO: 1725 - DNA, 20 bp, Glycine max
  primer
acagcatcaaccttagccgt

SEQ ID NO: 1726 - DNA, 20 bp, Glycine max
  primer
ttacaccccagctgttcctc

SEQ ID NO: 1727 - DNA, 20 bp, Glycine max
  primer
atgtgcccaattctgctacc

SEQ ID NO: 1728 - DNA, 20 bp, Glycine max
  primer
agttgctagttccggcaaga

SEQ ID NO: 1729 - DNA, 20 bp, Glycine max
  primer
gaccaatcattccaggcatt

SEQ ID NO: 1730 - DNA, 20 bp, Glycine max
  primer
gccgagagaggacaaacaaa

SEQ ID NO: 1731 - DNA, 20 bp, Glycine max
  primer
tgttgcttgtcttgctttgc

SEQ ID NO: 1732 - DNA, 20 bp, Glycine max
  primer
aagtgcggttttcaatgtcc

SEQ ID NO: 1733 - DNA, 20 bp, Glycine max
  primer
ttctgccctttctgatttcc

SEQ ID NO: 1734 - DNA, 20 bp, Glycine max
  primer
gccaagtaatgctccaccaa

SEQ ID NO: 1735 - DNA, 20 bp, Glycine max
  primer
gccatttctcttagggggtt

SEQ ID NO: 1736 - DNA, 19 bp, Glycine max
  primer
gggaaaggggtttcacaga

SEQ ID NO: 1737 - DNA, 20 bp, Glycine max
  primer
aagaccctgcgggctactat

SEQ ID NO: 1738 - DNA, 20 bp, Glycine max
  primer
aagctgaaccaagtgcctgt

SEQ ID NO: 1739 - DNA, 20 bp, Glycine max
  primer
gcaaattcatggaagaggga

SEQ ID NO: 1740 - DNA, 20 bp, Glycine max
  primer
aattgcttcctggaccgtaa

SEQ ID NO: 1741 - DNA, 20 bp, Glycine max
  primer
gatcactcagaatccagggc

SEQ ID NO: 1742 - DNA, 20 bp, Glycine max
  primer
gcatcgcatcagtacaacca

SEQ ID NO: 1743 - DNA, 20 bp, Glycine max
  primer
cattgcaaagcaagggtttt

SEQ ID NO: 1744 - DNA, 20 bp, Glycine max
  primer
acgcgattgagttttgatcc

SEQ ID NO: 1745 - DNA, 21 bp, Glycine max
  primer
tgagtcgatatgtttgtgcca

SEQ ID NO: 1746 - DNA, 21 bp, Glycine max
  primer
ccccctcgaggtattttatga

SEQ ID NO: 1747 - DNA, 20 bp, Glycine max
  primer
tcacgccatgtgctctactc

SEQ ID NO: 1748 - DNA, 20 bp, Glycine max
  primer
aggagagagacgccacagaa

SEQ ID NO: 1749 - DNA, 20 bp, Glycine max
  primer
tgttacttctggtggtcccc

SEQ ID NO: 1750 - DNA, 20 bp, Glycine max
  primer
ccagacagcgcaatgaaata

SEQ ID NO: 1751 - DNA, 20 bp, Glycine max
  primer
atgaatttggtcctttcgct

SEQ ID NO: 1752 - DNA, 21 bp, Glycine max
  primer
gtcatgcacctgcttcatatt

SEQ ID NO: 1753 - DNA, 20 bp, Glycine max
  primer
cggacgtcaagaacacaaga

SEQ ID NO: 1754 - DNA, 20 bp, Glycine max
  primer
attaggcgtattggtgaccg

SEQ ID NO: 1755 - DNA, 20 bp, Glycine max
  primer
ctgcaaagttgttgcttgga

SEQ ID NO: 1756 - DNA, 20 bp, Glycine max
  primer
tggaggataacacattcgca

SEQ ID NO: 1757 - DNA, 20 bp, Glycine max
  primer
caataaatgcacgcaacctg

SEQ ID NO: 1758 - DNA, 20 bp, Glycine max
  primer
ctgcacggtcaaagcatcta

SEQ ID NO: 1759 - DNA, 21 bp, Glycine max
  primer
ccagatcgaatcaatggaaag

SEQ ID NO: 1760 - DNA, 20 bp, Glycine max
  primer
taccaggctgcaatgcataa

SEQ ID NO: 1761 - DNA, 22 bp, Glycine max
  primer
caagcttttacaccagagcaga

SEQ ID NO: 1762 - DNA, 20 bp, Glycine max
  primer
tcgttgcccatcatagttca

SEQ ID NO: 1763 - DNA, 20 bp, Glycine max
  primer
gttccttctttggagttgcg

SEQ ID NO: 1764 - DNA, 20 bp, Glycine max
  primer
cttcaaagccaacagcaaca

SEQ ID NO: 1765 - DNA, 20 bp, Glycine max
  primer
attcttccatgatgggggtt

SEQ ID NO: 1766 - DNA, 20 bp, Glycine max
  primer
cctgagcaagagtggaggac

SEQ ID NO: 1767 - DNA, 20 bp, Glycine max
  primer
taccactctccacctccacc

SEQ ID NO: 1768 - DNA, 20 bp, Glycine max
  primer
ccatgttgtggattcagtgc

SEQ ID NO: 1769 - DNA, 22 bp, Glycine max
  primer
ttaagtctgaaactggaagtgc

SEQ ID NO: 1770 - DNA, 20 bp, Glycine max
  primer
cctctccacgttgttccttt

SEQ ID NO: 1771 - DNA, 20 bp, Glycine max
  primer
ccttgtttgtgtgttcaggc

SEQ ID NO: 1772 - DNA, 20 bp, Glycine max
  primer
ctttggcagattcgaggaag

SEQ ID NO: 1773 - DNA, 20 bp, Glycine max
  primer
tcaaccaaggacaattagca

SEQ ID NO: 1774 - DNA, 20 bp, Glycine max
  primer
gcacatcgtgactagcaggt

SEQ ID NO: 1775 - DNA, 22 bp, Glycine max
  primer
gcgacatcttggttcttatttg

SEQ ID NO: 1776 - DNA, 21 bp, Glycine max
  primer
aaggcatttttccttctctgg

SEQ ID NO: 1777 - DNA, 19 bp, Glycine max
  primer
ctgctgcagttggtaaccg

SEQ ID NO: 1778 - DNA, 20 bp, Glycine max
  primer
attccctcctccaaccatgt

SEQ ID NO: 1779 - DNA, 20 bp, Glycine max
  primer
ttcttttgtcgtctcggacc

SEQ ID NO: 1780 - DNA, 20 bp, Glycine max
  primer
ccctaaatcggaaccagaaa

SEQ ID NO: 1781 - DNA, 20 bp, Glycine max
  primer
gggggaaaacacccatgtat

SEQ ID NO: 1782 - DNA, 20 bp, Glycine max
  primer
ttccagaagacacaccaagc

SEQ ID NO: 1783 - DNA, 20 bp, Glycine max
  primer
ctgtgtgtttcgctccaaga

SEQ ID NO: 1784 - DNA, 20 bp, Glycine max
  primer
gggaatggatcccgaattat

SEQ ID NO: 1785 - DNA, 20 bp, Glycine max
  primer
tgggcttcctcaattacacc

SEQ ID NO: 1786 - DNA, 20 bp, Glycine max
  primer
gttgggatactgcattggct

SEQ ID NO: 1787 - DNA, 19 bp, Glycine max
  primer
gtccctggagctgatggat

SEQ ID NO: 1788 - DNA, 20 bp, Glycine max
  primer
tgggactcgatacaatgtgc

SEQ ID NO: 1789 - DNA, 20 bp, Glycine max
  primer
aggaggtgcctggtctgtta

SEQ ID NO: 1790 - DNA, 20 bp, Glycine max
  primer
acaacatggaaacctgctcc

SEQ ID NO: 1791 - DNA, 20 bp, Glycine max
  primer
catggggctcctttttgtta

SEQ ID NO: 1792 - DNA, 20 bp, Glycine max
  primer
ttcatccagctcatggacaa

SEQ ID NO: 1793 - DNA, 20 bp, Glycine max
  primer
gaattgctcggctcattttc

SEQ ID NO: 1794 - DNA, 20 bp, Glycine max
  primer
tgaaggcgaagagtctgacc

SEQ ID NO: 1795 - DNA, 20 bp, Glycine max
  primer
gcaaaccagcttctggagag

SEQ ID NO: 1796 - DNA, 20 bp, Glycine max
  primer
cgacaatcctgaacccaaat

SEQ ID NO: 1797 - DNA, 20 bp, Glycine max
  primer
tagtgaaagcacgagagcga

SEQ ID NO: 1798 - DNA, 20 bp, Glycine max
  primer
caagaacgaagctttgaccc

SEQ ID NO: 1799 - DNA, 20 bp, Glycine max
  primer
cggttacaatgggcttctgt

SEQ ID NO: 1800 - DNA, 20 bp, Glycine max
  primer
caggctggtgatgtcatttg

SEQ ID NO: 1801 - DNA, 20 bp, Glycine max
  primer
caacaaccacctccacaaaa

SEQ ID NO: 1802 - DNA, 20 bp, Glycine max
  primer
caacaccaatggagcttgtg

SEQ ID NO: 1803 - DNA, 20 bp, Glycine max
  primer
tttccgtgattttctgaccc

SEQ ID NO: 1804 - DNA, 20 bp, Glycine max
  primer
caccacgatatatggcagca

SEQ ID NO: 1805 - DNA, 20 bp, Glycine max
  primer
ctgcattctctgcaactcca

SEQ ID NO: 1806 - DNA, 20 bp, Glycine max
  primer
tctgaaattcggtgaggctt

SEQ ID NO: 1807 - DNA, 20 bp, Glycine max
  primer
aacaccttcaaagccaccac

SEQ ID NO: 1808 - DNA, 20 bp, Glycine max
  primer
tggatggaacagtggcatta

SEQ ID NO: 1809 - DNA, 20 bp, Glycine max
  primer
tgtggtgttgccagtggtat

SEQ ID NO: 1810 - DNA, 20 bp, Glycine max
  primer
gagaagaactcggtggcaag

SEQ ID NO: 1811 - DNA, 22 bp, Glycine max
  primer
tgatacagggaaagagagacgc

SEQ ID NO: 1812 - DNA, 19 bp, Glycine max
  primer
gacctgacccgacccaaat

SEQ ID NO: 1813 - DNA, 20 bp, Glycine max
  primer
accagcaaacaaaaactggg

SEQ ID NO: 1814 - DNA, 20 bp, Glycine max
  primer
catcacaaacaagctggtgg

SEQ ID NO: 1815 - DNA, 21 bp, Glycine max
  primer
ccagggatcatagatgtcgaa

SEQ ID NO: 1816 - DNA, 20 bp, Glycine max
  primer
tacagcacggaaccactagc

SEQ ID NO: 1817 - DNA, 20 bp, Glycine max
  primer
tgcagcttcacacacaatga

SEQ ID NO: 1818 - DNA, 20 bp, Glycine max
  primer
cttgggacttgttgaaggga

SEQ ID NO: 1819 - DNA, 20 bp, Glycine max
  primer
cgctggattgattctggagt

SEQ ID NO: 1820 - DNA, 20 bp, Glycine max
  primer
gcatgcatctaccaccacac

SEQ ID NO: 1821 - DNA, 20 bp, Glycine max
  primer
agttacaatgttggcgcctt

SEQ ID NO: 1822 - DNA, 20 bp, Glycine max
  primer
ggagctggttgagatggtgt

SEQ ID NO: 1823 - DNA, 20 bp, Glycine max
  primer
ttgtcatcacccatgaatcg

SEQ ID NO: 1824 - DNA, 20 bp, Glycine max
  primer
ttttggaaggcatttctgct

SEQ ID NO: 1825 - DNA, 20 bp, Glycine max
  primer
aattcccaagaatcccttgc

SEQ ID NO: 1826 - DNA, 19 bp, Glycine max
  primer
ccctcagttggtgctgatg

SEQ ID NO: 1827 - DNA, 20 bp, Glycine max
  primer
gcattctattgaagagcgcc

SEQ ID NO: 1828 - DNA, 20 bp, Glycine max
  primer
agcggtcatgggtatcaaag

SEQ ID NO: 1829 - DNA, 20 bp, Glycine max
  primer
tcacagggtgattggtgaaa

SEQ ID NO: 1830 - DNA, 20 bp, Glycine max
  primer
atgccaacccaagatatgga

SEQ ID NO: 1831 - DNA, 20 bp, Glycine max
  primer
aaaacctgtgttcactgggc

SEQ ID NO: 1832 - DNA, 20 bp, Glycine max
  primer
cagggcctatcagtgcaaat

SEQ ID NO: 1833 - DNA, 20 bp, Glycine max
  primer
agaaaaaggtcaagcgctca

SEQ ID NO: 1834 - DNA, 20 bp, Glycine max
  primer
agcgcttgttaggatgagga

SEQ ID NO: 1835 - DNA, 20 bp, Glycine max
  primer
caatctctccgcgttttctc

SEQ ID NO: 1836 - DNA, 20 bp, Glycine max
  primer
ttgaagtgcgaacaagaacg

SEQ ID NO: 1837 - DNA, 20 bp, Glycine max
  primer
ctttcagcagcagcaacaac

SEQ ID NO: 1838 - DNA, 20 bp, Glycine max
  primer
cggaacatcatttctgcttg

SEQ ID NO: 1839 - DNA, 20 bp, Glycine max
  primer
tccttggctctggaagagaa

SEQ ID NO: 1840 - DNA, 20 bp, Glycine max
  primer
tttggattctcagggtttgg

SEQ ID NO: 1841 - DNA, 20 bp, Glycine max
  primer
aaattttggaagtgggggac

SEQ ID NO: 1842 - DNA, 20 bp, Glycine max
  primer
ccaatcctgtggctgtataa

SEQ ID NO: 1843 - DNA, 20 bp, Glycine max
  primer
ctctcatccaaactgcctgg

SEQ ID NO: 1844 - DNA, 20 bp, Glycine max
  primer
tgctgaccgatacaaatgga

SEQ ID NO: 1845 - DNA, 20 bp, Glycine max
  primer
ttatcaccgatcctcatccc

SEQ ID NO: 1846 - DNA, 20 bp, Glycine max
  primer
caagatcaagccccatttgt

SEQ ID NO: 1847 - DNA, 20 bp, Glycine max
  primer
tggccaagagtcaacgacta

SEQ ID NO: 1848 - DNA, 22 bp, Glycine max
  primer
gtgatacacgcatcacgtaaaa

SEQ ID NO: 1849 - DNA, 22 bp, Glycine max
  primer
tctccttgatttccctctatcg

SEQ ID NO: 1850 - DNA, 20 bp, Glycine max
  primer
cgcaggttgctggttgttat

SEQ ID NO: 1851 - DNA, 22 bp, Glycine max
  primer
ctggttgtatgtgatatctcgg

SEQ ID NO: 1852 - DNA, 20 bp, Glycine max
  primer
accttcatatcgacagggca

SEQ ID NO: 1853 - DNA, 20 bp, Glycine max
  primer
ttaatgccccttcttcaacg

SEQ ID NO: 1854 - DNA, 20 bp, Glycine max
  primer
ctgcagtgaagttcggatca

SEQ ID NO: 1855 - DNA, 20 bp, Glycine max
  primer
tttcagccccaacttcagtc

SEQ ID NO: 1856 - DNA, 20 bp, Glycine max
  primer
gaaagggaaatccgtgtcaa

SEQ ID NO: 1857 - DNA, 20 bp, Glycine max
  primer
cgcaacaaacacatagccac

SEQ ID NO: 1858 - DNA, 20 bp, Glycine max
  primer
ctgccattttctcaccgatt

SEQ ID NO: 1859 - DNA, 20 bp, Glycine max
  primer
tttacattgcaaccaccacc

SEQ ID NO: 1860 - DNA, 20 bp, Glycine max
  primer
aagaaaggggaactgttggg

SEQ ID NO: 1861 - DNA, 20 bp, Glycine max
  primer
gataaccgtcactctgccgt

SEQ ID NO: 1862 - DNA, 20 bp, Glycine max
  primer
cagcatcttccaacacgaga

SEQ ID NO: 1863 - DNA, 20 bp, Glycine max
  primer
agaagtgaggctattgggca

SEQ ID NO: 1864 - DNA, 20 bp, Glycine max
  primer
cccagctcaagtcactctcc

SEQ ID NO: 1865 - DNA, 20 bp, Glycine max
  primer
ttgcagcttgcgtaatatcg

SEQ ID NO: 1866 - DNA, 20 bp, Glycine max
  primer
tgtgtcgtccattcgtcatt

SEQ ID NO: 1867 - DNA, 20 bp, Glycine max
  primer
tcatctccttactcagccgc

SEQ ID NO: 1868 - DNA, 19 bp, Glycine max
  primer
aaggtggagggaggttggt

SEQ ID NO: 1869 - DNA, 20 bp, Glycine max
  primer
gctccaaactcatcaaccgt

SEQ ID NO: 1870 - DNA, 20 bp, Glycine max
  primer
ttcaagagaaaaaccgtggg

SEQ ID NO: 1871 - DNA, 20 bp, Glycine max
  primer
ccatcacctgatatccccac

SEQ ID NO: 1872 - DNA, 20 bp, Glycine max
  primer
atgacccagagccaaaaaga

SEQ ID NO: 1873 - DNA, 21 bp, Glycine max
  primer
aaggtcgcatgaataagttcg

SEQ ID NO: 1874 - DNA, 20 bp, Glycine max
  primer
ccccctcgagtttttgtttt

SEQ ID NO: 1875 - DNA, 20 bp, Glycine max
  primer
gtttggaaacaaaaccgtgg

SEQ ID NO: 1876 - DNA, 20 bp, Glycine max
  primer
ggcaacaacacatggtgaag

SEQ ID NO: 1877 - DNA, 20 bp, Glycine max
  primer
tcaactgaaagcttcgagca

SEQ ID NO: 1878 - DNA, 20 bp, Glycine max
  primer
gtttccatccatgtcaccct

SEQ ID NO: 1879 - DNA, 20 bp, Glycine max
  primer
ttctacccagttttgcaccc

SEQ ID NO: 1880 - DNA, 20 bp, Glycine max
  primer
ttgcagggctgctactttct

SEQ ID NO: 1881 - DNA, 20 bp, Glycine max
  primer
aattctggctccgtgttagc

SEQ ID NO: 1882 - DNA, 20 bp, Glycine max
  primer
gctccctttaatgcccttct

SEQ ID NO: 1883 - DNA, 20 bp, Glycine max
  primer
cgatgtggatgtattggacg

SEQ ID NO: 1884 - DNA, 20 bp, Glycine max
  primer
tatatacctggggtgctgcg

SEQ ID NO: 1885 - DNA, 20 bp, Glycine max
  primer
gcaagcttttctctttggga

SEQ ID NO: 1886 - DNA, 20 bp, Glycine max
  primer
actcacccgcttcagttcct

SEQ ID NO: 1887 - DNA, 20 bp, Glycine max
  primer
gttattaccggtgcacccac

SEQ ID NO: 1888 - DNA, 20 bp, Glycine max
  primer
tgaatttgaatcgtcgcaag

SEQ ID NO: 1889 - DNA, 20 bp, Glycine max
  primer
actccttttcaaccccatcc

SEQ ID NO: 1890 - DNA, 20 bp, Glycine max
  primer
gaggaaattgagggagggac

SEQ ID NO: 1891 - DNA, 20 bp, Glycine max
  primer
tcagggatcctcatcctcac

SEQ ID NO: 1892 - DNA, 20 bp, Glycine max
  primer
tggataatattgttggcgca

SEQ ID NO: 1893 - DNA, 22 bp, Glycine max
  primer
gcatcggcaaatacttacacaa

SEQ ID NO: 1894 - DNA, 22 bp, Glycine max
  primer
cttggtcccattactcaatcaa

SEQ ID NO: 1895 - DNA, 20 bp, Glycine max
  primer
acgtacaccggagaccactc

SEQ ID NO: 1896 - DNA, 20 bp, Glycine max
  primer
gaagcaggagagtgacccag

SEQ ID NO: 1897 - DNA, 20 bp, Glycine max
  primer
tcggcacgagaaaacttctt

SEQ ID NO: 1898 - DNA, 20 bp, Glycine max
  primer
gggcatgatgtcctgaaact

SEQ ID NO: 1899 - DNA, 20 bp, Glycine max
  primer
tccttcccaacacaaacaca

SEQ ID NO: 1900 - DNA, 20 bp, Glycine max
  primer
tttctggaaaactccatccg

SEQ ID NO: 1901 - DNA, 20 bp, Glycine max
  primer
taagctcctgccttccagtg

SEQ ID NO: 1902 - DNA, 20 bp, Glycine max
  primer
ggtgcttcttgcaaaggttc

SEQ ID NO: 1903 - DNA, 20 bp, Glycine max
  primer
gcggtgagggtgtatctctt

SEQ ID NO: 1904 - DNA, 20 bp, Glycine max
  primer
cgcgcgttaataccacctat

SEQ ID NO: 1905 - DNA, 21 bp, Glycine max
  primer
cccaaacctctaaggacaacc

SEQ ID NO: 1906 - DNA, 20 bp, Glycine max
  primer
tgaccatgcaatgaaagagg

SEQ ID NO: 1907 - DNA, 20 bp, Glycine max
  primer
attctgatctcccaagcgaa

SEQ ID NO: 1908 - DNA, 21 bp, Glycine max
  primer
tgagtcatcgcgactagacaa

SEQ ID NO: 1909 - DNA, 20 bp, Glycine max
  primer
aaggaagcaagttgagcgaa

SEQ ID NO: 1910 - DNA, 20 bp, Glycine max
  primer
gagagggagggagtggttgt

SEQ ID NO: 1911 - DNA, 20 bp, Glycine max
  primer
ccacaccttgctgacacagt

SEQ ID NO: 1912 - DNA, 19 bp, Glycine max
  primer
atggaagtgatggctgctg

SEQ ID NO: 1913 - DNA, 20 bp, Glycine max
  primer
tcttccccaccaacagctac

SEQ ID NO: 1914 - DNA, 20 bp, Glycine max
  primer
tgctctaacataacctgcgg

SEQ ID NO: 1915 - DNA, 21 bp, Glycine max
  primer
cagctattgcttttgttccca

SEQ ID NO: 1916 - DNA, 22 bp, Glycine max
  primer
gagaaagagagagagggtccaa

SEQ ID NO: 1917 - DNA, 20 bp, Glycine max
  primer
acagcctgagaagttgcgat

SEQ ID NO: 1918 - DNA, 20 bp, Glycine max
  primer
actgtccatttggaacaccg

SEQ ID NO: 1919 - DNA, 19 bp, Glycine max
  primer
gattccccgtcaacctcag

SEQ ID NO: 1920 - DNA, 20 bp, Glycine max
  primer
tgagagggtggaggtgtagg

SEQ ID NO: 1921 - DNA, 21 bp, Glycine max
  primer
tgaaaaacttccctcttgtgc

SEQ ID NO: 1922 - DNA, 20 bp, Glycine max
  primer
tttccattgcaaaccaaaca

SEQ ID NO: 1923 - DNA, 20 bp, Glycine max
  primer
gatcacgagccctctctcac

SEQ ID NO: 1924 - DNA, 21 bp, Glycine max
  primer
cctaaatcctcagagctgcac

SEQ ID NO: 1925 - DNA, 20 bp, Glycine max
  primer
gagccaattgatcaacacga

SEQ ID NO: 1926 - DNA, 20 bp, Glycine max
  primer
tcactctcggcagcttttct

SEQ ID NO: 1927 - DNA, 20 bp, Glycine max
  primer
gcacttcgaattgtcgctgt

SEQ ID NO: 1928 - DNA, 20 bp, Glycine max
  primer
ctcaaaccaaagtgaagccc

SEQ ID NO: 1929 - DNA, 20 bp, Glycine max
  primer
aagcacattagattgcgtcg

SEQ ID NO: 1930 - DNA, 20 bp, Glycine max
  primer
tgtgacatcgcctcgagtaa

SEQ ID NO: 1931 - DNA, 20 bp, Glycine max
  primer
gatggttaccgatggaggaa

SEQ ID NO: 1932 - DNA, 20 bp, Glycine max
  primer
ttgcttcttcacattgcacc

SEQ ID NO: 1933 - DNA, 20 bp, Glycine max
  primer
ttggtcttcctcctttgtgg

SEQ ID NO: 1934 - DNA, 20 bp, Glycine max
  primer
aattcaccccaacaaccaaa

SEQ ID NO: 1935 - DNA, 20 bp, Glycine max
  primer
ttgcaaagtttagagaccaa

SEQ ID NO: 1936 - DNA, 21 bp, Glycine max
  primer
tgggttgacaaattagtcctt

SEQ ID NO: 1937 - DNA, 20 bp, Glycine max
  primer
ggacagggatgaggatgaaa

SEQ ID NO: 1938 - DNA, 20 bp, Glycine max
  primer
atacgaggatcctatggggc

SEQ ID NO: 1939 - DNA, 20 bp, Glycine max
  primer
gcaggaagggaatactgacg

SEQ ID NO: 1940 - DNA, 19 bp, Glycine max
  primer
cctacattccaggcccagt

SEQ ID NO: 1941 - DNA, 20 bp, Glycine max
  primer
ccctcagtcacagaaacagc

SEQ ID NO: 1942 - DNA, 20 bp, Glycine max
  primer
gctctactgcctcaaatggc

SEQ ID NO: 1943 - DNA, 20 bp, Glycine max
  primer
ggcacgagataaacggaagt

SEQ ID NO: 1944 - DNA, 20 bp, Glycine max
  primer
tcaggagtcttcccatccag

SEQ ID NO: 1945 - DNA, 20 bp, Glycine max
  primer
gggctcattttccccatatt

SEQ ID NO: 1946 - DNA, 20 bp, Glycine max
  primer
tattcaatagcgcagccctt

SEQ ID NO: 1947 - DNA, 20 bp, Glycine max
  primer
ttatcccaacgccttttctg

SEQ ID NO: 1948 - DNA, 20 bp, Glycine max
  primer
aggaagagccaaaacaccaa

SEQ ID NO: 1949 - DNA, 22 bp, Glycine max
  primer
tcgtgatgagagagtatcgctt

SEQ ID NO: 1950 - DNA, 20 bp, Glycine max
  primer
tccgtccagactgcacataa

SEQ ID NO: 1951 - DNA, 20 bp, Glycine max
  primer
aaaccacccaaggtgatctg

SEQ ID NO: 1952 - DNA, 20 bp, Glycine max
  primer
tgtcgcgaatcgtatgagaa

SEQ ID NO: 1953 - DNA, 20 bp, Glycine max
  primer
ctggtgtatcgtgtgcgtct

SEQ ID NO: 1954 - DNA, 20 bp, Glycine max
  primer
aaagggagaggttggtggtt

SEQ ID NO: 1955 - DNA, 20 bp, Glycine max
  primer
cgaaccgagtgctttcactt

SEQ ID NO: 1956 - DNA, 20 bp, Glycine max
  primer
atgatgcttctgggtaacgg

SEQ ID NO: 1957 - DNA, 20 bp, Glycine max
  primer
gaaggaagaaacaacgctcg

SEQ ID NO: 1958 - DNA, 20 bp, Glycine max
  primer
cgaaccagtgtcactagcca

SEQ ID NO: 1959 - DNA, 20 bp, Glycine max
  primer
tgcttcgtttgcacctaatg

SEQ ID NO: 1960 - DNA, 20 bp, Glycine max
  primer
cggccatagtgtctccactt

SEQ ID NO: 1961 - DNA, 20 bp, Glycine max
  primer
aaatggatcagcagagtggg

SEQ ID NO: 1962 - DNA, 20 bp, Glycine max
  primer
gggaggagtcatctgtggaa

SEQ ID NO: 1963 - DNA, 20 bp, Glycine max
  primer
caggaacagacatggcactg

SEQ ID NO: 1964 - DNA, 20 bp, Glycine max
  primer
tggacagttcctcagatccc

SEQ ID NO: 1965 - DNA, 20 bp, Glycine max
  primer
ggtgttggaaccataggcat

SEQ ID NO: 1966 - DNA, 20 bp, Glycine max
  primer
aagcattggaaccaggtgag

SEQ ID NO: 1967 - DNA, 20 bp, Glycine max
  primer
agctgctttaaggaacgtgg

SEQ ID NO: 1968 - DNA, 21 bp, Glycine max
  primer
gctttcatatggatgagctgc

SEQ ID NO: 1969 - DNA, 20 bp, Glycine max
  primer
agccagtagcctttctgcaa

SEQ ID NO: 1970 - DNA, 20 bp, Glycine max
  primer
acgtgacctttttcattgcc

SEQ ID NO: 1971 - DNA, 20 bp, Glycine max
  primer
aaggttgtgttgcgtcttca

SEQ ID NO: 1972 - DNA, 20 bp, Glycine max
  primer
aaggcataacacatctccgc

SEQ ID NO: 1973 - DNA, 20 bp, Glycine max
  primer
gctgaaattgcaactgggat

SEQ ID NO: 1974 - DNA, 20 bp, Glycine max
  primer
aaggttgtaagcaggccctt

SEQ ID NO: 1975 - DNA, 20 bp, Glycine max
  primer
tggtatccggctcatctttc

SEQ ID NO: 1976 - DNA, 20 bp, Glycine max
  primer
cggttcataaccctcatgct

SEQ ID NO: 1977 - DNA, 20 bp, Glycine max
  primer
gtgcaagagaaaccctctgc

SEQ ID NO: 1978 - DNA, 20 bp, Glycine max
  primer
cctagggcttgtgagtttgc

SEQ ID NO: 1979 - DNA, 23 bp, Glycine max
  primer
tggatgaagcaggatatagatgg

SEQ ID NO: 1980 - DNA, 20 bp, Glycine max
  primer
atcaacctacgcaccgctac

SEQ ID NO: 1981 - DNA, 20 bp, Glycine max
  primer
gccacttgtaccgcctgtta

SEQ ID NO: 1982 - DNA, 20 bp, Glycine max
  primer
ggggaattttcaggcaactc

SEQ ID NO: 1983 - DNA, 20 bp, Glycine max
  primer
gatctcaacttgccagctcc

SEQ ID NO: 1984 - DNA, 20 bp, Glycine max
  primer
acccaattgctgcagagaag

SEQ ID NO: 1985 - DNA, 21 bp, Glycine max
  primer
ttactccatcggtctctcgac

SEQ ID NO: 1986 - DNA, 19 bp, Glycine max
  primer
gtgagttcggtctccgaca

SEQ ID NO: 1987 - DNA, 20 bp, Glycine max
  primer
gagaaggggtagggatccag

SEQ ID NO: 1988 - DNA, 20 bp, Glycine max
  primer
caaggaggacatggagttgg

SEQ ID NO: 1989 - DNA, 20 bp, Glycine max
  primer
aatgtttcaagcaaccaggc

SEQ ID NO: 1990 - DNA, 20 bp, Glycine max
  primer
ttggctgtggaaaggttttt

SEQ ID NO: 1991 - DNA, 19 bp, Glycine max
  primer
tcaaggatgcctcggtcac

SEQ ID NO: 1992 - DNA, 21 bp, Glycine max
  primer
tcatgctgtagaaggtgctga

SEQ ID NO: 1993 - DNA, 21 bp, Glycine max
  primer
ttggacttggagttacacctg

SEQ ID NO: 1994 - DNA, 22 bp, Glycine max
  primer
agaaaaagaagctgaggtggtg

SEQ ID NO: 1995 - DNA, 20 bp, Glycine max
  primer
aatgcaacctcgttttcgtc

SEQ ID NO: 1996 - DNA, 20 bp, Glycine max
  primer
tatgatccaaccttgccctc

SEQ ID NO: 1997 - DNA, 23 bp, Glycine max
  primer
caattgcagaaggtagatgagtc

SEQ ID NO: 1998 - DNA, 22 bp, Glycine max
  primer
gccaattgtactgtttggtttg

SEQ ID NO: 1999 - DNA, 20 bp, Glycine max
  primer
gggattcaaggtccacttca

SEQ ID NO: 2000 - DNA, 20 bp, Glycine max
  primer
gcgagagacaggaggaagaa

SEQ ID NO: 2001 - DNA, 20 bp, Glycine max
  primer
taagcctaggccacgaagaa

SEQ ID NO: 2002 - DNA, 20 bp, Glycine max
  primer
accccaacctgcactatctg

SEQ ID NO: 2003 - DNA, 20 bp, Glycine max
  primer
gggtaacctcgtcatcaacg

SEQ ID NO: 2004 - DNA, 20 bp, Glycine max
  primer
tggtccactcacacaggaag

SEQ ID NO: 2005 - DNA, 20 bp, Glycine max
  primer
tccctcggctcaaatatcac

SEQ ID NO: 2006 - DNA, 20 bp, Glycine max
  primer
cccttaatagggttgggctt

SEQ ID NO: 2007 - DNA, 20 bp, Glycine max
  primer
gccagtccaactgtgacctt

SEQ ID NO: 2008 - DNA, 20 bp, Glycine max
  primer
tcatcgggcatgaaaggtat

SEQ ID NO: 2009 - DNA, 20 bp, Glycine max
  primer
ggtccaccttcttcctcctc

SEQ ID NO: 2010 - DNA, 20 bp, Glycine max
  primer
aaacagtgctctcggatgct

SEQ ID NO: 2011 - DNA, 20 bp, Glycine max
  primer
gaaaatggggtggctaacaa

SEQ ID NO: 2012 - DNA, 20 bp, Glycine max
  primer
gagagagacacaacctcggc

SEQ ID NO: 2013 - DNA, 20 bp, Glycine max
  primer
agaagcttgtggtggaggag

SEQ ID NO: 2014 - DNA, 20 bp, Glycine max
  primer
gaccaacaaggagctggtgt

SEQ ID NO: 2015 - DNA, 22 bp, Glycine max
  primer
ttttctagctaccctagcgaat

SEQ ID NO: 2016 - DNA, 21 bp, Glycine max
  primer
gctggctattaatcccacgta

SEQ ID NO: 2017 - DNA, 20 bp, Glycine max
  primer
atcctggctgctcattatgg

SEQ ID NO: 2018 - DNA, 20 bp, Glycine max
  primer
ctgtacccaaaggaggtgga

SEQ ID NO: 2019 - DNA, 20 bp, Glycine max
  primer
tttccggactactcagcagg

SEQ ID NO: 2020 - DNA, 20 bp, Glycine max
  primer
tgaggattttcaatcatggg

SEQ ID NO: 2021 - DNA, 20 bp, Glycine max
  primer
cccaccaaggtttgtaatgc

SEQ ID NO: 2022 - DNA, 20 bp, Glycine max
  primer
gcagcacctgaaattaggga

SEQ ID NO: 2023 - DNA, 20 bp, Glycine max
  primer
gtggtgcagctgggaataat

SEQ ID NO: 2024 - DNA, 20 bp, Glycine max
  primer
catggatgcaatttccaatg

SEQ ID NO: 2025 - DNA, 22 bp, Glycine max
  primer
catggagtgatcttgttgttgc

SEQ ID NO: 2026 - DNA, 22 bp, Glycine max
  primer
caacaagccttaacgagacaga

SEQ ID NO: 2027 - DNA, 20 bp, Glycine max
  primer
ggtgatggcgagttgaaagt

SEQ ID NO: 2028 - DNA, 20 bp, Glycine max
  primer
aacccttggagttgctgatg

SEQ ID NO: 2029 - DNA, 20 bp, Glycine max
  primer
agcatctatcacggccaatc

SEQ ID NO: 2030 - DNA, 20 bp, Glycine max
  primer
aaaggcaaaagagccatcaa

SEQ ID NO: 2031 - DNA, 20 bp, Glycine max
  primer
ctagccacaagaagcccaag

SEQ ID NO: 2032 - DNA, 20 bp, Glycine max
  primer
ccatgccacaaattgaacac

SEQ ID NO: 2033 - DNA, 20 bp, Glycine max
  primer
cgaactccgttggagaaaag

SEQ ID NO: 2034 - DNA, 20 bp, Glycine max
  primer
aggcttggcaaaaagtctca

SEQ ID NO: 2035 - DNA, 20 bp, Glycine max
  primer
aagcttctgctttgcctgag

SEQ ID NO: 2036 - DNA, 22 bp, Glycine max
  primer
tctccacttcaaggaatatcca

SEQ ID NO: 2037 - DNA, 20 bp, Glycine max
  primer
cacctccgttgttgttgttg

SEQ ID NO: 2038 - DNA, 20 bp, Glycine max
  primer
caaatgggttccaccagaag

SEQ ID NO: 2039 - DNA, 20 bp, Glycine max
  primer
ggagttcgcctagttcctga

SEQ ID NO: 2040 - DNA, 20 bp, Glycine max
  primer
ctcataattcgatgggtcgc

SEQ ID NO: 2041 - DNA, 20 bp, Glycine max
  primer
ggttgcacttgacttgggtt

SEQ ID NO: 2042 - DNA, 20 bp, Glycine max
  primer
aatgtcctggtcccacaaag

SEQ ID NO: 2043 - DNA, 20 bp, Glycine max
  primer
aagaaaggcttttgcagcat

SEQ ID NO: 2044 - DNA, 21 bp, Glycine max
  primer
tgaggacaatttttcccacac

SEQ ID NO: 2045 - DNA, 20 bp, Glycine max
  primer
ggaagtaacagcgttggagg

SEQ ID NO: 2046 - DNA, 20 bp, Glycine max
  primer
cccactcattcccctcacta

SEQ ID NO: 2047 - DNA, 20 bp, Glycine max
  primer
caagctttgggaggacacat

SEQ ID NO: 2048 - DNA, 20 bp, Glycine max
  primer
ctgctgccagaactcatcaa

SEQ ID NO: 2049 - DNA, 20 bp, Glycine max
  primer
cctcctgttagggtggtgaa

SEQ ID NO: 2050 - DNA, 20 bp, Glycine max
  primer
agctccacctccagcagtta

SEQ ID NO: 2051 - DNA, 20 bp, Glycine max
  primer
caacgatgccaccaacatag

SEQ ID NO: 2052 - DNA, 20 bp, Glycine max
  primer
tagcggtgatagcagtggtg

SEQ ID NO: 2053 - DNA, 20 bp, Glycine max
  primer
gtttgggacatcatcgtcgt

SEQ ID NO: 2054 - DNA, 21 bp, Glycine max
  primer
cgttggcatgtgtaaatgatg

SEQ ID NO: 2055 - DNA, 20 bp, Glycine max
  primer
ttcatgtgaatggctttgga

SEQ ID NO: 2056 - DNA, 20 bp, Glycine max
  primer
aagctttgctattccgggtt

SEQ ID NO: 2057 - DNA, 20 bp, Glycine max
  primer
ccttggattggacaaccatc

SEQ ID NO: 2058 - DNA, 20 bp, Glycine max
  primer
gaccaggaccaccacctcta

SEQ ID NO: 2059 - DNA, 20 bp, Glycine max
  primer
aaatgacaagcctttgtggc

SEQ ID NO: 2060 - DNA, 20 bp, Glycine max
  primer
tggatgaccttgtttcagca

SEQ ID NO: 2061 - DNA, 20 bp, Glycine max
  primer
tgaagttcatgctctgcacc

SEQ ID NO: 2062 - DNA, 20 bp, Glycine max
  primer
ttggatgacactaaaggggc

SEQ ID NO: 2063 - DNA, 20 bp, Glycine max
  primer
gaccccagtgtgatgttgaa

SEQ ID NO: 2064 - DNA, 20 bp, Glycine max
  primer
atgcctttttgacgagcaat

SEQ ID NO: 2065 - DNA, 20 bp, Glycine max
  primer
aggatttgtgacaagcgtgg

SEQ ID NO: 2066 - DNA, 20 bp, Glycine max
  primer
aggaacacaaactcgccaat

SEQ ID NO: 2067 - DNA, 19 bp, Glycine max
  primer
tttcagcaatggcagagcc

SEQ ID NO: 2068 - DNA, 20 bp, Glycine max
  primer
agtgaagctttggagggaga

SEQ ID NO: 2069 - DNA, 20 bp, Glycine max
  primer
gaaccgtcaaggtttttgga

SEQ ID NO: 2070 - DNA, 20 bp, Glycine max
  primer
acagtttcatcgcgatcctt

SEQ ID NO: 2071 - DNA, 20 bp, Glycine max
  primer
actctcagaattccatcgcc

SEQ ID NO: 2072 - DNA, 20 bp, Glycine max
  primer
atcgagtgtttgcttcgctt

SEQ ID NO: 2073 - DNA, 20 bp, Glycine max
  primer
tcgcggtactcttcgaattt

SEQ ID NO: 2074 - DNA, 20 bp, Glycine max
  primer
caagccattcccaaccataa

SEQ ID NO: 2075 - DNA, 20 bp, Glycine max
  primer
agagcagtggcagtggaaat

SEQ ID NO: 2076 - DNA, 20 bp, Glycine max
  primer
cacatgatccaccaaagcag

SEQ ID NO: 2077 - DNA, 20 bp, Glycine max
  primer
atagcacgagggtggttacg

SEQ ID NO: 2078 - DNA, 20 bp, Glycine max
  primer
tgccatctttccaaacaaca

SEQ ID NO: 2079 - DNA, 20 bp, Glycine max
  primer
tcacctcagttgcttcaacg

SEQ ID NO: 2080 - DNA, 20 bp, Glycine max
  primer
aaacactttgcattccctgg

SEQ ID NO: 2081 - DNA, 20 bp, Glycine max
  primer
taaggcctgagagtttccga

SEQ ID NO: 2082 - DNA, 20 bp, Glycine max
  primer
cccactaacagagcaggagg

SEQ ID NO: 2083 - DNA, 20 bp, Glycine max
  primer
tgaactgatgtcagggtcca

SEQ ID NO: 2084 - DNA, 20 bp, Glycine max
  primer
tagcgagacagacccacctt

SEQ ID NO: 2085 - DNA, 20 bp, Glycine max
  primer
aattgggaagggtgtgtgaa

SEQ ID NO: 2086 - DNA, 20 bp, Glycine max
  primer
gatttggatcgattcgtgct

SEQ ID NO: 2087 - DNA, 20 bp, Glycine max
  primer
ccgccattccctttattgta

SEQ ID NO: 2088 - DNA, 20 bp, Glycine max
  primer
gggcctaaaaaccatggaaa

SEQ ID NO: 2089 - DNA, 20 bp, Glycine max
  primer
ttgtaacccgattcttggga

SEQ ID NO: 2090 - DNA, 20 bp, Glycine max
  primer
agtttccagaaaggcctggt

SEQ ID NO: 2091 - DNA, 20 bp, Glycine max
  primer
aaaatgccaagagttggctg

SEQ ID NO: 2092 - DNA, 20 bp, Glycine max
  primer
tacttctgcgagcattgtgc

SEQ ID NO: 2093 - DNA, 20 bp, Glycine max
  primer
tgatgtggctgaaaatggag

SEQ ID NO: 2094 - DNA, 20 bp, Glycine max
  primer
aagattcttttccggccatt

SEQ ID NO: 2095 - DNA, 21 bp, Glycine max
  primer
cttgtcacaacatcaccgtgt

SEQ ID NO: 2096 - DNA, 20 bp, Glycine max
  primer
tgtttgcactgttcccaact

SEQ ID NO: 2097 - DNA, 20 bp, Glycine max
  primer
agtaatcgaaccccagaccc

SEQ ID NO: 2098 - DNA, 20 bp, Glycine max
  primer
aaactctgcccctgtagcaa

SEQ ID NO: 2099 - DNA, 20 bp, Glycine max
  primer
tctcgatttcatcgccttct

SEQ ID NO: 2100 - DNA, 20 bp, Glycine max
  primer
aacctgcaagtttgaccacc

SEQ ID NO: 2101 - DNA, 20 bp, Glycine max
  primer
cacagatatggaggcggtct

SEQ ID NO: 2102 - DNA, 20 bp, Glycine max
  primer
tttgaaggccctcccttatt

SEQ ID NO: 2103 - DNA, 20 bp, Glycine max
  primer
ttttggcaaaggctctgtct

SEQ ID NO: 2104 - DNA, 20 bp, Glycine max
  primer
ctgctcaggcaaaccagaat

SEQ ID NO: 2105 - DNA, 20 bp, Glycine max
  primer
gatagatcaggctcctcccc

SEQ ID NO: 2106 - DNA, 20 bp, Glycine max
  primer
tcctcatgggaatggaaaag

SEQ ID NO: 2107 - DNA, 20 bp, Glycine max
  primer
gataggacagccagaatgcc

SEQ ID NO: 2108 - DNA, 20 bp, Glycine max
  primer
atggcaactcttccagcaat

SEQ ID NO: 2109 - DNA, 22 bp, Glycine max
  primer
ttttgatggcaactgttcaaag

SEQ ID NO: 2110 - DNA, 20 bp, Glycine max
  primer
atggggtgagcacaaaagag

SEQ ID NO: 2111 - DNA, 20 bp, Glycine max
  primer
gaagatggcaaggtccttca

SEQ ID NO: 2112 - DNA, 20 bp, Glycine max
  primer
gattgaccccatttgaccac

SEQ ID NO: 2113 - DNA, 20 bp, Glycine max
  primer
gctcttcctctttctgccct

SEQ ID NO: 2114 - DNA, 19 bp, Glycine max
  primer
aatgccactcgcaacaaag

SEQ ID NO: 2115 - DNA, 21 bp, Glycine max
  primer
tctgatgtcttttcagttgcg

SEQ ID NO: 2116 - DNA, 20 bp, Glycine max
  primer
tgaagcaccttctcagtcca

SEQ ID NO: 2117 - DNA, 20 bp, Glycine max
  primer
ttccagtctgggttctcctg

SEQ ID NO: 2118 - DNA, 20 bp, Glycine max
  primer
aagagcaaacagctgcatca

SEQ ID NO: 2119 - DNA, 20 bp, Glycine max
  primer
tgctcctgcctttgattctt

SEQ ID NO: 2120 - DNA, 20 bp, Glycine max
  primer
tgtagctccatctcctggct

SEQ ID NO: 2121 - DNA, 20 bp, Glycine max
  primer
ccatggatggagcagctgta

SEQ ID NO: 2122 - DNA, 20 bp, Glycine max
  primer
ataaccaagaagcattgcca

SEQ ID NO: 2123 - DNA, 20 bp, Glycine max
  primer
gattttcccattgcctgaga

SEQ ID NO: 2124 - DNA, 21 bp, Glycine max
  primer
gcagcatgaattcagaccact

SEQ ID NO: 2125 - DNA, 20 bp, Glycine max
  primer
gattccactgttccctccaa

SEQ ID NO: 2126 - DNA, 20 bp, Glycine max
  primer
aggcatagtagtccctgcca

SEQ ID NO: 2127 - DNA, 20 bp, Glycine max
  primer
tgctcctcaaggaaggaaaa

SEQ ID NO: 2128 - DNA, 20 bp, Glycine max
  primer
ggtcaggataccactgggtg

SEQ ID NO: 2129 - DNA, 21 bp, Glycine max
  primer
gccaggtaacatgaaatccag

SEQ ID NO: 2130 - DNA, 20 bp, Glycine max
  primer
cattgccggagatgtacaga

SEQ ID NO: 2131 - DNA, 20 bp, Glycine max
  primer
gacccgaccaaccttaaaca

SEQ ID NO: 2132 - DNA, 20 bp, Glycine max
  primer
tcttgggccaaagcaaatac

SEQ ID NO: 2133 - DNA, 20 bp, Glycine max
  primer
tgtcatgcgatcgaaatgtt

SEQ ID NO: 2134 - DNA, 20 bp, Glycine max
  primer
ttgtgaattgcatctctcgc

SEQ ID NO: 2135 - DNA, 20 bp, Glycine max
  primer
taaccgtaggtgaacggctc

SEQ ID NO: 2136 - DNA, 20 bp, Glycine max
  primer
cgaagacggagcagaaaagt

SEQ ID NO: 2137 - DNA, 20 bp, Glycine max
  primer
agaggagcgagtccaatctg

SEQ ID NO: 2138 - DNA, 20 bp, Glycine max
  primer
gagtaactgtgcgcaaacga

SEQ ID NO: 2139 - DNA, 20 bp, Glycine max
  primer
aatatggaacagaagccccc

SEQ ID NO: 2140 - DNA, 20 bp, Glycine max
  primer
cgcgatgggaagattattgt

SEQ ID NO: 2141 - DNA, 20 bp, Glycine max
  primer
gagggagatttgtgaaggca

SEQ ID NO: 2142 - DNA, 20 bp, Glycine max
  primer
acacacgagcattgaactcg

SEQ ID NO: 2143 - DNA, 20 bp, Glycine max
  primer
ggattgctgttgtgtcagga

SEQ ID NO: 2144 - DNA, 20 bp, Glycine max
  primer
tatcgcagtaccctcgcttc

SEQ ID NO: 2145 - DNA, 20 bp, Glycine max
  primer
ttcaccccatgtttatcgtg

SEQ ID NO: 2146 - DNA, 20 bp, Glycine max
  primer
ggtgatgatgggttaaggga

SEQ ID NO: 2147 - DNA, 20 bp, Glycine max
  primer
ccaaccagctcttctccaag

SEQ ID NO: 2148 - DNA, 20 bp, Glycine max
  primer
tctggcacagaacagaggtg

SEQ ID NO: 2149 - DNA, 20 bp, Glycine max
  primer
ttacactgttgaacgcagcc

SEQ ID NO: 2150 - DNA, 20 bp, Glycine max
  primer
atgaccctttgagcacaacc

SEQ ID NO: 2151 - DNA, 20 bp, Glycine max
  primer
tgtagcctaacccctccctt

SEQ ID NO: 2152 - DNA, 20 bp, Glycine max
  primer
cgtcacatgctcttgcagtt

SEQ ID NO: 2153 - DNA, 22 bp, Glycine max
  primer
cacaacacaacaattccaacct

SEQ ID NO: 2154 - DNA, 20 bp, Glycine max
  primer
atttgcaatattgtggggga

SEQ ID NO: 2155 - DNA, 20 bp, Glycine max
  primer
ataccgatatgatcggcgag

SEQ ID NO: 2156 - DNA, 20 bp, Glycine max
  primer
ctttgaaaggggaatgctga

SEQ ID NO: 2157 - DNA, 20 bp, Glycine max
  primer
tttgctttcaaatgtggctg

SEQ ID NO: 2158 - DNA, 20 bp, Glycine max
  primer
ctccacctgatgcacttctg

SEQ ID NO: 2159 - DNA, 20 bp, Glycine max
  primer
ccaacctttctgcagcattt

SEQ ID NO: 2160 - DNA, 21 bp, Glycine max
  primer
cctgttcactctgacaggctc

SEQ ID NO: 2161 - DNA, 20 bp, Glycine max
  primer
aacaagatccttgcaccacc

SEQ ID NO: 2162 - DNA, 20 bp, Glycine max
  primer
actttaagccaccacatggc

SEQ ID NO: 2163 - DNA, 20 bp, Glycine max
  primer
aaactgttcttcgacggagc

SEQ ID NO: 2164 - DNA, 20 bp, Glycine max
  primer
gctccactttaaccgtgacc

SEQ ID NO: 2165 - DNA, 20 bp, Glycine max
  primer
ggagggtctgaatccaactg

SEQ ID NO: 2166 - DNA, 20 bp, Glycine max
  primer
gacccgaaaccaaattcaaa

SEQ ID NO: 2167 - DNA, 20 bp, Glycine max
  primer
ggcttgcattgaatggtttt

SEQ ID NO: 2168 - DNA, 20 bp, Glycine max
  primer
ctatatgggcaacactgggg

SEQ ID NO: 2169 - DNA, 20 bp, Glycine max
  primer
tgctggttcgtacccttttc

SEQ ID NO: 2170 - DNA, 20 bp, Glycine max
  primer
accgatggcatctgagaaac

SEQ ID NO: 2171 - DNA, 20 bp, Glycine max
  primer
ctctagctccaccacgaacc

SEQ ID NO: 2172 - DNA, 20 bp, Glycine max
  primer
aaaccttgggaaaggaacac

SEQ ID NO: 2173 - DNA, 20 bp, Glycine max
  primer
tgccaaaagggaactgaaac

SEQ ID NO: 2174 - DNA, 19 bp, Glycine max
  primer
catcacccccagtttcctc

SEQ ID NO: 2175 - DNA, 20 bp, Glycine max
  primer
tgacccaaacctatgtgcaa

SEQ ID NO: 2176 - DNA, 20 bp, Glycine max
  primer
ggcattatgctgttgagggt

SEQ ID NO: 2177 - DNA, 20 bp, Glycine max
  primer
tgttccacttgatcagcagc

SEQ ID NO: 2178 - DNA, 20 bp, Glycine max
  primer
ggtggtggcagagttttgtt

SEQ ID NO: 2179 - DNA, 20 bp, Glycine max
  primer
catttcccggtgttgaaatc

SEQ ID NO: 2180 - DNA, 20 bp, Glycine max
  primer
cattgcgtcttctggagtca

SEQ ID NO: 2181 - DNA, 20 bp, Glycine max
  primer
agcaccttccaacaacaacc

SEQ ID NO: 2182 - DNA, 20 bp, Glycine max
  primer
ccatgtatagggccaaggaa

SEQ ID NO: 2183 - DNA, 20 bp, Glycine max
  primer
cctcaaggaagaaggaaccc

SEQ ID NO: 2184 - DNA, 20 bp, Glycine max
  primer
ggttcggtagctcagcaaag

SEQ ID NO: 2185 - DNA, 20 bp, Glycine max
  primer
ctaggcaacgagccaaaaag

SEQ ID NO: 2186 - DNA, 20 bp, Glycine max
  primer
tatggtgactactcgcacgc

SEQ ID NO: 2187 - DNA, 20 bp, Glycine max
  primer
tgatgatcctggaggaaagg

SEQ ID NO: 2188 - DNA, 20 bp, Glycine max
  primer
actctgtgcaatgcttgtgg

SEQ ID NO: 2189 - DNA, 20 bp, Glycine max
  primer
gcttcccggtttttgaattt

SEQ ID NO: 2190 - DNA, 20 bp, Glycine max
  primer
cccactgaaacaggtccatt

SEQ ID NO: 2191 - DNA, 20 bp, Glycine max
  primer
attacgggaaagtgcgactg

SEQ ID NO: 2192 - DNA, 20 bp, Glycine max
  primer
tccgcaaccataattgtgac

SEQ ID NO: 2193 - DNA, 20 bp, Glycine max
  primer
tgaagaaagaggaggagcca

SEQ ID NO: 2194 - DNA, 20 bp, Glycine max
  primer
gctttcaaggactgagaccg

SEQ ID NO: 2195 - DNA, 20 bp, Glycine max
  primer
aaagaaacgggcatatggtg

SEQ ID NO: 2196 - DNA, 20 bp, Glycine max
  primer
gcctttccatcattctccac

SEQ ID NO: 2197 - DNA, 20 bp, Glycine max
  primer
gggtaatttgggggaaaaga

SEQ ID NO: 2198 - DNA, 20 bp, Glycine max
  primer
tatgttccgtggcgtacaaa

SEQ ID NO: 2199 - DNA, 20 bp, Glycine max
  primer
cacgcgatgtttggctacta

SEQ ID NO: 2200 - DNA, 20 bp, Glycine max
  primer
gaggacggaccgtatgtgac

SEQ ID NO: 2201 - DNA, 19 bp, Glycine max
  primer
gtcttcagctcctcctcgg

SEQ ID NO: 2202 - DNA, 20 bp, Glycine max
  primer
tccccagtgatcctcatttc

SEQ ID NO: 2203 - DNA, 20 bp, Glycine max
  primer
cttcctcagggaacagtcca

SEQ ID NO: 2204 - DNA, 20 bp, Glycine max
  primer
gagaggagtcttggtggtgc

SEQ ID NO: 2205 - DNA, 20 bp, Glycine max
  primer
gttgcacccagaaaatgctt

SEQ ID NO: 2206 - DNA, 20 bp, Glycine max
  primer
caggcattgcatagggtctt

SEQ ID NO: 2207 - DNA, 20 bp, Glycine max
  primer
gttgcacccagaaaatgctt

SEQ ID NO: 2208 - DNA, 20 bp, Glycine max
  primer
caggcattgcatagggtctt

SEQ ID NO: 2209 - DNA, 20 bp, Glycine max
  primer
tggagatttgatgaagccaa

SEQ ID NO: 2210 - DNA, 20 bp, Glycine max
  primer
gcactcaaactgccacaaga

SEQ ID NO: 2211 - DNA, 20 bp, Glycine max
  primer
cccacactttttggtcctca

SEQ ID NO: 2212 - DNA, 20 bp, Glycine max
  primer
ttaggaaaggggagggaaaa

SEQ ID NO: 2213 - DNA, 20 bp, Glycine max
  primer
gggctcgtaggtaacgtcag

SEQ ID NO: 2214 - DNA, 20 bp, Glycine max
  primer
gtcatagccggcgaattaag

SEQ ID NO: 2215 - DNA, 20 bp, Glycine max
  primer
tggaattcgacaaaggaagg

SEQ ID NO: 2216 - DNA, 20 bp, Glycine max
  primer
gctatgcaacgtgtttccct

SEQ ID NO: 2217 - DNA, 20 bp, Glycine max
  primer
gagtggcaggatagtccagg

SEQ ID NO: 2218 - DNA, 20 bp, Glycine max
  primer
ctctctccttatccgctccc

SEQ ID NO: 2219 - DNA, 20 bp, Glycine max
  primer
gctagcttctggggagccta

SEQ ID NO: 2220 - DNA, 20 bp, Glycine max
  primer
caggttgtgaggcattttga

SEQ ID NO: 2221 - DNA, 20 bp, Glycine max
  primer
ccagagttggctgttccatt

SEQ ID NO: 2222 - DNA, 21 bp, Glycine max
  primer
agcttcctcagtcaaatgtgc

SEQ ID NO: 2223 - DNA, 20 bp, Glycine max
  primer
actggtttgccacaaggaac

SEQ ID NO: 2224 - DNA, 20 bp, Glycine max
  primer
tcccgaaggaaagcactcta

SEQ ID NO: 2225 - DNA, 20 bp, Glycine max
  primer
ccttgagctgagttctggct

SEQ ID NO: 2226 - DNA, 20 bp, Glycine max
  primer
ggttttcatgatgaccctgg

SEQ ID NO: 2227 - DNA, 20 bp, Glycine max
  primer
catcgtcatcttgatcgtcc

SEQ ID NO: 2228 - DNA, 20 bp, Glycine max
  primer
aagtccagctctaagcagcg

SEQ ID NO: 2229 - DNA, 20 bp, Glycine max
  primer
acaaggctgataggaagcga

SEQ ID NO: 2230 - DNA, 20 bp, Glycine max
  primer
ttccttgtttcttggccatc

SEQ ID NO: 2231 - DNA, 21 bp, Glycine max
  primer
gcaacagatgtcaaatagccg

SEQ ID NO: 2232 - DNA, 21 bp, Glycine max
  primer
aagctttacaaacccatgacg

SEQ ID NO: 2233 - DNA, 20 bp, Glycine max
  primer
ttttaatggggtctggcaac

SEQ ID NO: 2234 - DNA, 20 bp, Glycine max
  primer
acgcgttagttctgcttcgt

SEQ ID NO: 2235 - DNA, 20 bp, Glycine max
  primer
gttatcaaaaggaccgtggc

SEQ ID NO: 2236 - DNA, 20 bp, Glycine max
  primer
ttgccttgcttccttgttct

SEQ ID NO: 2237 - DNA, 20 bp, Glycine max
  primer
gaggcctccaatgtaatcca

SEQ ID NO: 2238 - DNA, 20 bp, Glycine max
  primer
tctcttccttgggaagcaac

SEQ ID NO: 2239 - DNA, 20 bp, Glycine max
  primer
tcttcttgtggtgcttgtgc

SEQ ID NO: 2240 - DNA, 20 bp, Glycine max
  primer
gttgcggtaaccacaggaat

SEQ ID NO: 2241 - DNA, 20 bp, Glycine max
  primer
ctttggagatcccatcatgc

SEQ ID NO: 2242 - DNA, 20 bp, Glycine max
  primer
cgttgagcttctggtggaat

SEQ ID NO: 2243 - DNA, 20 bp, Glycine max
  primer
gcgcacattgttctgcttta

SEQ ID NO: 2244 - DNA, 20 bp, Glycine max
  primer
tccttgctcaagttcaacca

SEQ ID NO: 2245 - DNA, 20 bp, Glycine max
  primer
tcacggttcgtactgacgag

SEQ ID NO: 2246 - DNA, 20 bp, Glycine max
  primer
agtgctccacccattgttgt

SEQ ID NO: 2247 - DNA, 20 bp, Glycine max
  primer
caatgctgcgtctcacttgt

SEQ ID NO: 2248 - DNA, 20 bp, Glycine max
  primer
catacatgaatggggcctct

SEQ ID NO: 2249 - DNA, 20 bp, Glycine max
  primer
ctaccacaactaggagccgc

SEQ ID NO: 2250 - DNA, 20 bp, Glycine max
  primer
cattatcacggcttgcagaa

SEQ ID NO: 2251 - DNA, 20 bp, Glycine max
  primer
caatgccgattactctccgt

SEQ ID NO: 2252 - DNA, 20 bp, Glycine max
  primer
gagacggaacctccgagtct

SEQ ID NO: 2253 - DNA, 20 bp, Glycine max
  primer
tttacagttccagcacagcg

SEQ ID NO: 2254 - DNA, 20 bp, Glycine max
  primer
attatgcaagagaatgcccg

SEQ ID NO: 2255 - DNA, 20 bp, Glycine max
  primer
aggtcacgggaggaagattt

SEQ ID NO: 2256 - DNA, 20 bp, Glycine max
  primer
gagatgggtgctaggcatgt

SEQ ID NO: 2257 - DNA, 20 bp, Glycine max
  primer
tgaaacttccaggccaaaac

SEQ ID NO: 2258 - DNA, 20 bp, Glycine max
  primer
agcgaaattcgggaaagact

SEQ ID NO: 2259 - DNA, 20 bp, Glycine max
  primer
aaataggggcattgatgacg

SEQ ID NO: 2260 - DNA, 19 bp, Glycine max
  primer
ttccaatcccggtccatag

SEQ ID NO: 2261 - DNA, 20 bp, Glycine max
  primer
acattcatgcccccatctaa

SEQ ID NO: 2262 - DNA, 20 bp, Glycine max
  primer
cgcaacacaacatatgctcc

SEQ ID NO: 2263 - DNA, 20 bp, Glycine max
  primer
catctccaacgtctcggttt

SEQ ID NO: 2264 - DNA, 20 bp, Glycine max
  primer
cctgcaaagaagcttgatga

SEQ ID NO: 2265 - DNA, 21 bp, Glycine max
  primer
agaccagttttggcattgaga

SEQ ID NO: 2266 - DNA, 21 bp, Glycine max
  primer
ttccaagcgtgtttaccagtc

SEQ ID NO: 2267 - DNA, 20 bp, Glycine max
  primer
ttgagctaggtttgacggct

SEQ ID NO: 2268 - DNA, 20 bp, Glycine max
  primer
tggatttgtccaaggtgtga

SEQ ID NO: 2269 - DNA, 20 bp, Glycine max
  primer
tggcatcaaaaaggagaaca

SEQ ID NO: 2270 - DNA, 20 bp, Glycine max
  primer
tgaatgctggcatcgtaaag

SEQ ID NO: 2271 - DNA, 20 bp, Glycine max
  primer
tattggtccagttttgggga

SEQ ID NO: 2272 - DNA, 20 bp, Glycine max
  primer
caaccttccaatatccctgg

SEQ ID NO: 2273 - DNA, 20 bp, Glycine max
  primer
tgccagtcaggatcagtttg

SEQ ID NO: 2274 - DNA, 20 bp, Glycine max
  primer
cccagatagcattgaaggga

SEQ ID NO: 2275 - DNA, 20 bp, Glycine max
  primer
acgtgaccataacaacgggt

SEQ ID NO: 2276 - DNA, 20 bp, Glycine max
  primer
gtgcaccgttgacaaagcta

SEQ ID NO: 2277 - DNA, 20 bp, Glycine max
  primer
gggaggccatactcatcaga

SEQ ID NO: 2278 - DNA, 20 bp, Glycine max
  primer
aactcaggtggatgattcgc

SEQ ID NO: 2279 - DNA, 20 bp, Glycine max
  primer
caattacaccgagcatcacg

SEQ ID NO: 2280 - DNA, 20 bp, Glycine max
  primer
atcatcgctcatcgtgtcag

SEQ ID NO: 2281 - DNA, 20 bp, Glycine max
  primer
tctctcccgctaaggtacga

SEQ ID NO: 2282 - DNA, 20 bp, Glycine max
  primer
accattgcatccaacaatga

SEQ ID NO: 2283 - DNA, 20 bp, Glycine max
  primer
tccccaaggaagcgtaaata

SEQ ID NO: 2284 - DNA, 20 bp, Glycine max
  primer
acgttcggctacatcaaagc

SEQ ID NO: 2285 - DNA, 20 bp, Glycine max
  primer
ttaattgctgagcagggacc

SEQ ID NO: 2286 - DNA, 20 bp, Glycine max
  primer
ttgcagcagtgcgataattc

SEQ ID NO: 2287 - DNA, 20 bp, Glycine max
  primer
tctggctctcttggaattgg

SEQ ID NO: 2288 - DNA, 20 bp, Glycine max
  primer
gatcgggtgatagttcacgg

SEQ ID NO: 2289 - DNA, 20 bp, Glycine max
  primer
ggcttgcatcttttggttct

SEQ ID NO: 2290 - DNA, 20 bp, Glycine max
  primer
tccctcatctgcaattttcc

SEQ ID NO: 2291 - DNA, 20 bp, Glycine max
  primer
agtgcctcctctgctatgga

SEQ ID NO: 2292 - DNA, 20 bp, Glycine max
  primer
caagcaattgaagcactgga

SEQ ID NO: 2293 - DNA, 20 bp, Glycine max
  primer
tgttttgttggcatggagaa

SEQ ID NO: 2294 - DNA, 20 bp, Glycine max
  primer
agctgaaactacctcgccaa

SEQ ID NO: 2295 - DNA, 20 bp, Glycine max
  primer
tctcatcctgttttctgccc

SEQ ID NO: 2296 - DNA, 20 bp, Glycine max
  primer
tgacatccttgacgtggaaa

SEQ ID NO: 2297 - DNA, 20 bp, Glycine max
  primer
tctcctcggttaaaggggtt

SEQ ID NO: 2298 - DNA, 20 bp, Glycine max
  primer
gcacccagtatcgcagtgta

SEQ ID NO: 2299 - DNA, 1106 bp, Glycine max
tttcacttcttcttcttcttcttcttcaatatcttggttctttgatattattctattgagcaaaaaatgggagttccagagagagaccctcttgcacaattgagtttgcctcctgggtttaggttttaccccacagatgaggagcttttggttcagtacctttgccgcaaggttgctggccatcatttctctcttccaatcattgctgaagttgatttgtacaagtttgatccatgggttcttccaggtaaggcagtgtttggagagaaggagtggtacttttttagcccaagagacaggaagtacccgaatggttcacgaccaaacagagtcgcgggttctgggtattggaaagcaactggaacagacaagatcatcaccactgaaggtagaaaagttggcataaaaaaagcacttgttttctacattggcaaagcacccaaaggctccaaaaccaattggatcatgcacgagtatcgccttctcgattcttcccgcaaacacaacctcggaaccgcaaagcttgatgattgggttctatgtcgtatctataagaagaactcaagttcacaaaaggttgaggctaattttttggctatggaatgcagcaatgggtcatcaccttcttcatcgtcccacgtggatgacatgctgggatcgttgccggagataaatgaccggtgcttcaccctgccacgagtgaactcactcagaacaatgcaccagcaggatgagaaattcgggtctccgaacatgggatccgggtttttctcggattgggttaactcgaccgatctcgattcgatttccgaattcgagtcgggttgccaaacccaaagaatggtgaattatgattgcaatgacttttttgttccttctctgccgcccttgggccatgtggactacatggtggatgcacctttggaggaggaggttcaaagtggtgtgagaacccgacgggtcgacgggccggggcattttcaaccgaatccagatacccgattgttaccgggctcaggtgacccattcgggtttgggtttattatgggtcagcaagttgagttcgggtttagggactgatagcgttgttctgtaaataattgggg

SEQ ID NO: 2300 - DNA, 1152 bp, Glycine max
ttccttgcaccttcttctttgaagatcgaaattgtgtttttgaaaaaatgggagttccagaggaagaccctctttcccaattgagtttgcctcctggttttcggttttaccccaccgacgaggagctcctcgttcagtatctatgccgcaaggtcgctggccaccatttctctcttccaatcattgctgaaattgatttgtacaagttcgacccatgggttcttccaagtaaggcgattttcggtgagaaagagtggtactttttcagccctagagacaggaaataccctaacgggtcccgacccaacagagtagccgggtcgggttattggaaagccaccggaaccgacaagatcatcaccaccgaaggtagaaaagttggcataaaaaaagccctcgttttctacattggcaaagcccccaaaggcaccaaaaccaattggatcatgcacgagtatcgcctcctcgactcttcccgaaaaaacaccggcaccaagcttgatgactgggttctgtgtcgtatctacaagaagaactcgagtgcacagaaggcggtgcaaaacggcgtcgttccgagcaacgagcacactcaatacagcaacggttcctcttcctcttcttcgtcacaactcgacgacgttctggaatcgctgccagcgattgatgaacggtgtttcccgatgccacgtgtcaacacgctgcagcaacagcagcacgaggagaaggtcaatgttcagaacttgggtgaaggggggttactggattggaccaacccttcggttctgaattcggtcgttgatttcgtatcggggaataataatcataatcaattggtgcaggaccagacgcaaggcatggtgaactacaacgcgtgcaatgacctctatgtccctgcgttatgccatgtgggcacgtcagttccgcaaaagatggaggaagaggtgcaaagcggcgtgagaaaccaacgggtccagaacaattcctggtttcttcagaatgatttcacacaggggtttcagaattcggttgacacgtctgggtttaagtacccggttcaaccggtggggttcgggttcaggaattgaaccctgttatcgattaataatagtagtaatcttttaaatcggtaaataggtggggattctttgggaa

SEQ ID NO: 2301 - DNA, 519 bp, Glycine max
atgaagagaggcagagaagagagtaagttagacatggctaattgcttgatgctattgacgaaagttggagagagtgaaaccaattacccaatatcaaagggtagtgatattggtgatttcaagtgcaagacttgcaatagaaggttctcttcttttcaagcccttggtggccatagagcaagccacaaaaaaccaaagctcatggtcacagatctttcgtgccatcaagagttaccgaacccaaccatgaaacaacaacctaggatgcacccgtgtccgatttgtgggcttgagtttgctattggacaagctttgggagggcacatgagaaagcatagaactgctattaatgatggcttgttgtgtggtaaaccttcttcttcgttgtccatcttgaaggaatcatcgaaagatggtgatcaaaagttgaatttgcgcttggacttgaacttgacgccattggaggaggatgatcttaagctcaacctaaggacgcccgtgctcaattgtttcatttga

SEQ ID NO: 2302 - DNA, 969 bp, Glycine max
atgggagaggaaacaactgagagtgagaaggaagatggagtgaccaatcttgatactgtctcaagaccaggggaagtgtctattaacccctctaccaagaaggaggatcttacattgaaaacttcaaaaaagaccgaacaagaacaaggtgaaaattcccaagataaaacactgagaaagccggacaaaatactgccttgcccccgctgcaatagcatggataccaagttctgctactacaataattacaatgtcaaccaaccccgccatttctgcaagaattgtcagagatactggactgccggtggagtgatgaggaatgttcctgtgggtgctggtcgcaggaagaacaagaattttacttctcagtatcgtcagataactgttcctgaagcagcactcttgagttctcagccggacttgccaaatggtccttccttgaactgtaatgccacactccttgcatttgggtctgatacacccttgtgtgaatcaatgacatctgttttgaaccttgctgaaaaagcagtgaacaattccacaacaaatggatttaatggctcagaagaactagaaattccttttgcttatgttgctggagaaaaagtgaatgatgattccaataaatcttctgacatgtcaacaaagccaatggaaggtgaaaccaccaataggtcccaagaacaagtcatagagaattgtcatcccttcccacctcatgctccctactttcctggtgccccttggcctttcccatggaatccagcactgtggaactccccggtaccatcaccacctgcttttttcccacaaggatttaccatgcctctctatcctgcagcagcttattggggtcttactataccgggtgcatggagcatcccatggctagcgcaaccatcattaccaaatagcacagtgtctaactctggtccagaccccattgcccgaaccatagccatgtcttga

(19) United States
(12) Patent Application Publication
(10) Pub. No.: US 2012/0198587 A1
(43) Pub. Date: Aug. 2, 2012

(54) SOYBEAN TRANSCRIPTION FACTORS AND OTHER GENES AND METHODS OF THEIR USE

(75) Inventors: Henry T. Nguyen, Columbia, MO (US); Gary Stacey, Columbia, MO (US); Dong Xu, Columbia, MO (US); Jianlin Cheng, Columbia, MO (US); Trupti Joshi, Columbia, MO (US); Marc Libault, Norman, OK (US); Babu Valliyodan, Columbia, MO (US)

(73) Assignee: The Curators of the University of Missouri

(21) Appl. No.: 13/381,448

(22) PCT Filed: Jun. 30, 2010
(86) PCT No.: PCT/US10/40687

§ 371 Date: Apr. 17, 2012

Related U.S. Application Data
(60) Provisional application No. 61/270,204, filed on Jun. 30, 2009.

(51) Int. Cl. C12N 15/82; A01H 5/00

(52) U.S. Cl. 800/287; 800/278; 800/289; 800/298; 800/312; 800/320.1; 800/320.3; 800/320.2; 800/314

(57) ABSTRACT

Gene expression is controlled at the transcriptional level by very diverse group of proteins called transcription factors (TFs). 5671 soybean (Glycine max) genes have been identified and disclosed as putative transcription factors through mining of soybean genome sequences. Distinct classes of the TFs are also disclosed which may be expressed and or function in a manner that is tissue specific, developmental stage specific, biotic and/or abiotic stress specific. Manipulation and/or genetic engineering of specific transcription factors may improve the agronomic performance or nutritional quality of plants. Transgenic plants expressing a select number of these TFs are disclosed. These transgenic plants show some promising traits, such as improving the capability of the plant to grow and reproduce under drought conditions.

### RELATED APPLICATIONS

This application claims priority to U.S. Provisional Application No. 61/270,204 filed Jun. 30, 2009, the contents of which are hereby incorporated into this application by reference.

### BACKGROUND

1. Field of the Invention

The present invention relates to methods and materials for identifying genes and the regulatory networks that control gene expression in an organism. More particularly, the present invention relates to soybean genes encoding transcription factors or other functional proteins that are expressed in a tissue specific, developmental stage specific, or biotic and abiotic stress specific manner.

2. Description of the Related Art

Gene expression is controlled at the transcriptional level by a very diverse group of proteins called transcription factors (TF or TFs). These proteins identify specific promoters of the genes regulated by them, and through protein-DNA and/or protein-protein interactions, these TFs help to assemble the basal transcription machinery in the cell. Transcription factors are master controllers in many living cells. They control or influence many biological processes, including cell cycle progression, metabolism, growth, development, reproduction, and responses to the environment. (Czechowski et al. 2004).

TFs play critical roles in all aspects of a higher plant's life cycle. Although several studies have analyzed the function of individual TFs, collectively these studies have provided information on only a few TFs. Therefore, it is important to identify and to understand the functions of more TFs in order to dissect their specific role in plant development, stress tolerance and plant-microbe interaction.c

Molecular tailoring of novel TFs, for example, has the potential to overcome a number of limitations in creating transgenic soybean plants with stress tolerance and better yield. A number of published reports show that genetic engineering of plants, both monocot and dicot, to modify gene expression can lead to enhanced stress tolerance. For example, over-expression of different types of TFs, such as DREB1A, ANAC, MYB, MYC and ZFHD in Arabidopsis strongly improved the drought and salt tolerance of transgenic plants (Liu et al. 1998; Abe et al. 2003; Tran et al. 2007).

Recently, introduction of SNAC 1 and ZmNF-YB2 TFs into rice and maize, respectively, enhanced the drought tolerance of transgenic plants, as demonstrated by field studies. Transgenic rice over-expressing the SNAC1 gene had 22-34% higher seed set than a negative control in the field under severe drought stress conditions at the reproductive stage, whereas transgenic maize over-expressing the ZmNF-YB2 gene (from Monsanto) produced a ˜50% increase in yield, relative to the controls, when water was withheld from the planted field area during the late vegetative stage (Hu et al. 2006; Nelson et al. 2007). The regulations forcing the listing or banning of trans-fats have spurred the development of low-linolenic soybeans. Recently, some modified zinc finger TFs (ZFP-TFs) that can specifically down-regulate the expression of the endogenous soybean FAD2-1 gene, which catalyzes the conversion of oleic acid to linoleic acid, were introduced into soybean. Seed-specific expression of these ZFP-TFs in transgenic soybean somatic embryos repressed FAD2-1 transcription and increased significantly the levels of oleic acid, indicating that engineering of TFs is capable of regulating fatty acid metabolism and modulating the expression of endogenous genes in plants (Wu et al. 2004).

Other studies have demonstrated the role of TFs during legume nodulation by characterizing mutant plant phenotypes. For example, The Medicago truncatula MtNSP1 and MtNSP2 genes encode two GRAS family TFs (Catoira et al., 2000; Oldroyd and Long, 2003; Kalo et al., 2005; Smit et al., 2005) that are essential for nodule development. MtERN, a member of the ETHYLENE RESPONSIVE FACTOR (ERF) family (Middleton et al., 2007), was shown to play a key role in the initiation and the maintenance of rhizobial infection. The Lotus japonicus NIN gene encodes a putative TF gene (Schauser et al., 1999). Mutants in the L. japonicus nm gene or the Pisum sativum ortholog (i.e. Sym35) failed to support rhizobial infection and did not show cortical cell division upon inoculation (Schauser et al., 1999; Borisov et al., 2003). In contrast, the L. japonicus astray mutant exhibited hypernodulation. The ASTRAY gene encodes for a bZIP TF (Nishimura et al., 2002).

DNA microarray analysis allows fast and simultaneous measurement of the expression levels of thousands of genes in a single experiment. However, current DNA microarray technology fails to accurately measure the expression levels of genes expressed at very low levels. For example, TFs are often missed in DNA microarray analysis due to the very low levels they are usually expressed in cells.

Drought is one of the major abiotic stress factors limiting crop productivity worldwide. Global climate changes may further exacerbate the drought situation in major crop-producing countries. Although irrigation may in theory solve the drought problem, it is usually not a viable option because of the cost associated with building and maintaining an effective irrigation system, as well as other non-economical issues, such as the general availability of water (Boyer, 1983). Thus, alternative means for alleviating plant water stress are needed.

In soybean, drought stress during flowering and early pod development significantly increases the rate of flower and pod abortion, thus decreasing final yield (Boyer 1983; Westgate and Peterson 1993). Soybean yield reduction of 40% because of drought is common experience among soybean producers in the United States (Muchow & Sinclair, 1986; Specht et al. 1999).

Mechanisms for selecting drought tolerant plants fall into three general categories. The first is called drought escape, in which selection is aimed at those developmental and maturation traits that match seasonal water availability with crop needs. The second is dehydration avoidance, in which selection is focused on traits that: lessen evaporatory water loss from plant surfaces or maintain water uptake during drought via a deeper and more extensive root system. The last mechanism is dehydration tolerance, in which selection is directed at maintaining cell turgor or enhancing cellular constituents that protect cytoplasmic proteins and membranes from drying.

The molecular mechanisms of abiotic stress responses and the genetic regulatory networks of drought stress tolerance have been reviewed recently (Wang et at 2003; Vinocur and Altman 2005; Chaves and Oliveira 2004; Shinozaki et al. 2003). Plant modification for enhanced drought tolerance is mostly based on the manipulation of either transcription and/or signaling factors or genes that directly protect plant cells against water deficit. Despite much progress in the field, understanding the basic biochemical and molecular mechanisms for drought stress perception, transduction, response and tolerance remains a major challenge in the field. Utilization of the knowledge on drought tolerance to generate plants that can tolerate extreme water deficit condition is even a bigger challenge.

Analysis of changes in gene expression within a target plant is important for revealing the transcriptional regulatory networks. Elucidation of these complex regulatory networks may contribute to our understanding of the responses mounted by a plant to various stresses and developmental changes, which may ultimately lead to crop improvement. DNA microarray assays (Schena et al 1995; Shalon et al. 1996) have provided an unprecedented opportunity for the generation of gene expression data on a whole-genome scale.

Gene expression profiling using cDNAs or oligonucleotides microarray technology has advanced our understanding of gene regulatory network when a plant is subject to various stresses (Bray 2004; Denby and Gehring 2005). For example, numerous genes that respond to dehydration stress have been identified in Arabidopsis and have been categorized as “rd” (responsive to dehydration) or “erd” (early response to dehydration) (Shinozaki and Yamaguchi-Shinozaki 1999).

There are at least four independent regulatory pathways for gene expression in response to water stress. Out of the four pathways, two are abscisic acid (ABA) dependent and the other two are ABA independent (Shinozaki and Yamaguchi-Shinozaki 2000). In the ABA independent regulatory pathways, a cis-acting element is involved and the Dehydration-responsive element/C-repeat (DRE/CRT) has been identified. DRE/CRT also functions in cold response and high-salt-responsive gene expression. When the DRE/CRT binding protein DREB1/ICBF is overexpressed in a transgenic Arabidopsis plant, changes in expression of more than 40 stress-inducible genes can be observed, which lead to enhanced tolerance to freeze, high salt, and drought (Seki et al, 2001; Fowler and Thomashow 2002; Murayama et al. 2004).

The production of microarrays and the global transcript profiling of plants have revolutionized the study of gene expression which provides a unique snapshot of how these plants are responding to a particular stress. However, no transcriptional profiling or transcriptome changes have been reported for soybean plants under various stress conditions, such as drought, flooding, disease infections, etc. There is also a lack of knowledge with respect to tissue specific expression of soybean genes and regulation of gene expression during different stage of soybean growth or reproduction. Moreover, no studies have systematically classified soybean TFs based on the structure of these proteins.

### SUMMARY

The instrumentalities described herein overcome the problems outlined above and advance the art by providing genes and DNA regulatory elements which may play an important role in regulating the growth and reproduction of a plant under normal or distress such as drought conditions, among others. Methodology is also provided whereby these genes responsive to various distress conditions may be introduced into a host plant to enhance its capability to grow and reproduce under such conditions. The regulatory elements may also be employed to control expression of heterologous genes which may be beneficial for enhancing a plant's capability to grow under such conditions.

Expression of many plant proteins are regulated by a group of proteins termed transcription factors (TFs). The expression of TFs may themselves be regulated. TF genes are generally expressed at relatively low levels which makes the detection and quantitation of their expression difficult. Quantitative reverse transcriptase-polymerase chain reaction (qRT-PCR) is the most sensitive technology currently available to quantify gene expression. High-throughput qRT-PCR has been used in several other plant species (e.g. A. thaliana, O. sativa and M. truncatula) to quantitate the expression of TF genes. See Czechowski T, Bari R P, Stitt M, Scheible W R, Udvardi M K (2004) Plant J 38: 366-379; Caldana C, Scheible W R, Mueller-Roeber B, Ruzicic S (2007). Plant Methods 3: 7; and Kakar K, Wandrey M, Czechowski T, Gaertner T, Scheible W R, Stitt M, Torres-Jerez I, Xiao Y, Redman J C, Wu H C, Cheung F, Town C D, Udvardi M K (2008) Plant Methods 4: 18.

It is also disclosed here a library of primers specifically designed for transcription factors (TF) In one embodiment, qRT-PCR may be used to profile gene expression in various soybean tissues using the primers specific for these genes. In another embodiment, the same primers may be used to identified genes whose expression levels change during various developmental or reproductive stages, such as during nodulation by rhizobia in roots, under drought stress, under flooding, or in developing seeds. Among the variety of results obtained was the identification of a number of transcription factors that are specifically expressed in soybean tissues, such as leaves, seeds, roots, etc.

In addition to qRT-PCR, high-through-put sequencing technologies (Illumina-Solexa) may be used to profile gene expression. Compared to more conventional high-through-put technologies (e.g. DNA microarray hybridization), Illumina-Solexa sequencing is more sensitive and allows full coverage of all genes expressed. qRT-PCR and high-through-put sequencing may also be combined to quantify low expressed genes such as TF genes. Using the most sensitive technologies available (i.e. qRT-PCR and high-through-put sequencing technologies (Illumina-Solexa)), a large number of TF genes have been identified and disclosed herein which may prove important in response to various environmental stresses, or to control plant development.

In one embodiment, microarray experiments may be conducted to analyze the gene expression pattern in soybean root and leaf tissues in response to drought stress. Tissue specific transcriptomes may be compared to help elucidate the transcriptional regulatory network and facilitate the identification of stress specific genes and promoters.

In another embodiment, a number of soybean TFs are shown to be expressed only in certain soybean tissues but not in others. These TFs may play an important role in regulating gene expression within the specific tissues. The DNA elements, responsible for tissue specific expression of these genes may be used to control the expression of other genes. Such DNA elements may include but are not limited to a promoter, an enhancer, etc. For instance, sometimes it may be desirable to express a plant transgene only in certain tissues, but not in others. To accomplish this goal, a transgene from the same or different plant may be placed under control of a tissue-specific promoter in order to drive the expression of the gene only in the certain tissues.

In another embodiment, certain soybean TF genes are expressed during seeding, or only at specific stage during seeding (termed “TFIS” for “TF implicated in seeding”). These TFs may play a role in seed filling and may function to control seed compositions. In one aspect, manipulation of these TFs through gene overexpression, gene silencing, or transgenic expression may prove useful in controlling the number, size or composition of the seeds.

In one embodiment, a method is disclosed for generating a transgenic plant from a host plant to create a transgenic plant that is more tolerant to an adverse condition when compared to the host plant. The method may include a step of altering the expression levels of a transcription factor or fragment thereof, and the adverse condition may be selected from one or more of an environmental conditions, such as, by way of example, too high or too low of water, salt, acidity, temperature or combination thereof. Preferably, the transcription factor has been shown to be upregulated or downregulated in an organism in response to the adverse condition, more preferably, by at least two fold. In another aspect, the organism is a second plant that is different from the host plant.

In one aspect, the transcription factor may be endogenous or exogenous to the host plant. “Exogenous” means the transcription factor is from a plant that is genetically different from the host plant. “Endogenous” means that the transcription factor is from the host plant.

In one embodiment, the transcription factor is encoded by a coding sequence such as polynucleotide sequence of SEQ ID. No. 2299, SEQ ID. No. 2300, SEQ ID. No. 2301, SEQ ID. No. 2302, or other transcription factors that are inducible by the adverse condition or those that may regulate expression of proteins that play a role in plant response to the adverse condition.

In another embodiment, the regulatory sequence in the genes encoding the transcription factors of this disclosure may be operably linked to a coding sequence to promote the expression of such coding sequence. Preferably, such coding sequence encode a protein that play a role in plant response to the adverse condition.

In another embodiment, some plant TF genes are induced by drought (these genes are termed DRG or TFIRD) or flooding stress (termed TFIRF). These TFs may help mobilize or activate proteins in plants in response to the drought or flooding conditions.

For purpose of this disclosure, genes whose expression are either up- or down-regulated in response to drought condition are referred to as Drought Response Genes (or DRGs). A DRG that is a transcription factor is also termed “Transcription factors in response to drought” (“TFIRD”). For purpose of this disclosure, a “DRG protein” refers to a protein encoded by a DRG. Some DRGs may show tissue specific expression patterns in response to drought condition. A transcription factor that is induced by flooding is termed “TFIRF” for “Transcription factors in response to Flooding.”

It is to be recognized that although the present disclosure primarily uses drought as an example of environmental distress, the methodology disclosed herein to identify plant genes that are upregulated or downregulated in response to various environmental stimuli and the methodology to manipulate such genes to enhance a plant's capability to growth under stress are applicable to other situations such as flooding, infection, etc.

The microarray experiments described in this disclosure may not have uncovered all the DRGs in all plants, or even in soybean alone, due to the variations in experimental conditions, and more importantly, due to the different gene expressions among different plant species. It is also to be understood that certain DRGs or TFs disclosed here may have been identified and studied previously; however, regulation of their expression under drought condition or their role in drought response may not have been appreciated in previous studies. Alternatively, some DRGs or TFs may contain novel coding sequences. Thus, it is an object of the present disclosure to identify known or unknown genes whose expression levels are altered in response to drought condition.

In order to generate a transgenic plant that is more tolerant to drought condition when compared to a host plant, the expression levels of a protein encoded by an endogenous Drought Response Gene (DRG) or a fragment thereof may be altered to confer a drought resistant phenotype to the host plant. More particularly, the transcription, translation or protein stability of the protein encoded by the DRG or TF may be modified so that the levels of this protein are rendered significantly higher than the levels of this protein would otherwise be even under the same drought condition. To this end, either the coding or non-coding regions, or both, of the endogenous DRG or TF may be modified.

In another aspect, in order to generate a transgenic plant that is more tolerant to drought condition when compared to a host plant, the method may comprise the steps of: (a) introducing into a plant cell a construct comprising a Drought Response Gene (DRG) or a fragment thereof encoding a polypeptide; and (b) generating a transgenic plant expressing said polypeptide or a fragment thereof. In one embodiment, the Drought Response Gene or a fragment thereof is derived from a plant that is genetically different from the host plant. In another embodiment, the Drought Response Gene or a fragment thereof is derived from a plant that belongs to the same species as the host plant. For instance, a DRG identified in soybean may be introduced into soybean as a transgene to confer upon the host increased capability to grow and/or reproduced under mild to severe drought conditions.

The DRGs or TFs disclosed here include known genes as well as genes whose functions are not yet fully understood. Nevertheless, both known or unknown DRGs or TFs may be placed under control of a promoter and be transformed into a host plant in accodance with standard plant transformation protocols. The transgenic plants thus obtained may be tested for the expression of the DRGs or TFs and their capability to grow and/or reproduce under drought conditions as compared to the original host (or parental) plant.

Although the TFs or DRGs disclosed herein are identified in soybean, they may be introduced into other plants as transgenes. Examples of such other plants may include corn, wheat, rice, cotton, sugar cane, or Arabidopsis. In another aspect, homologs in other plant species may be identified by PCR, hybridization or by genome search which may share substantial sequence similarity with the DRGs or TFs disclosed herein. In a preferred embodiment, such a homolog shares at least 90%, more preferably 98%, or even more preferably 99% sequence identity with a protein encoded by a soybean DRG or TF.

In another embodiment, a portion of the DRGs disclosed herein are transcription factors, such as most of the DRGs or fragments thereof listed in Table 6. Conversely, a portion of the TFs disclosed herein are DRGs. It is desirable to introduce one or more of these DRGs or fragments thereof into a host plant so that the transcription factors may be expressed at a sufficiently high level to drive the expression of other downstream effector proteins that may result in increased drought resistance to the transgenic plant.

It is further an object to identify the non-coding sequences of the DRGs, termed Drought Response Regulatory Elements (DRREs) for purpose of this disclosure. These DRREs may be used to prepare DNA constructs for the expression of genes of interest in a host plant. The DREEs or the DRGs may also be used to screen for factors or chemicals that may affect the expression of certain DRGs by interacting with a DREE. Such factors or chemicals may be used to induce drought responses by activating expression of certain genes in a plant.

For purpose of this disclosure, the genes of interest may be genes from other plants or even non-plant organisms. The genes of interest may be those identified and listed in this disclosure, or they may be any other genes that have been found to enhance the capability of a host plant to grow under water deficit condition.

In a preferred embodiment, the genes of interest may be placed under control of the DRREs such that their expression may be upregulated under drought condition. This arrangement is particularly useful for those genes of interest that may not be desirable under normal conditions, because such genes may be placed under a tightly regulated DRRE which only drives the expression of the genes of interest when water deficit condition is sensed by the plant. Under control of such a DRRE, expression of the gene of interest may be only detected under drought condition.

It is an object of this disclosure to provide a system and a method for the genetic modification of a plant, to increase the resistance of the plant to adverse conditions such as drought and/or excessive temperatures, compared to an unmodified plant.

It is another object of the present invention to provide a transgenic plant that exhibits increased resistance to adverse conditions such as drought and/or excessive temperatures as compared to an unmodified plant.

It is another object of the present invention to provide a system and method of modifying a plant, to alter the metabolism or development of the plant.

In one embodiment, a gene of interest may be placed under control of a tissue specific promoter such that such gene of interest may be expressed in specific site, for example, the guard cells. The expression of the introduced genes may enhance the capacity of a plant to modulate guard cell activity in response to water stress. For instance, the transgene may help reduce stomatal water loss. In addition, other characteristics such as early maturation of plants may be introduced into plants to help cope with drought condition.

Preferably, the transgene is under control of a promoter, which may be a constitutive or inducible promoter. An inducible promoter is inactive under normal condition, and is activated under certain conditions to drive the expression of the gene under its control. Conditions that may activate a promoter include but are not limited to light, heat, certain nutrients or chemicals, and water conditions. A promoter that is activated under water deficit condition is preferred.

In another aspect, a tissue specific promoter, an organ specific promoter, or a cell-specific promoter may be employed to control the transgene. Despite their different names, these promoters are similar in that they are only activated in certain cell, tissue or organ types. It is to be understood that a gene under control of an inducible promoter, or a promoter specific for certain cells, tissues or organs may have low level of expression even under conditions that are not supposed to activate the promoter, a phenomenon known as “leaky expression” in the field. A promoter can be both inducible and tissue specific. By way of example, a transgene may be placed under control of a guard cell specific promoter such that the gene can be inducibly expressed in the guard cell of the transgenic plant.

In another aspect, the present disclosure provides a method of generating a transgenic plant having an altered stress response or an altered phenotype compared to an unmodified plant. The coding sequences of the genes that are disclosed to be upregulated may be placed under a promoter such that the genes can be expressed in the transgenic plant. The method may contain two steps: (a) introducing into a plant cell capable of being transformed and regenerated into a whole plant a construct comprising, in addition to the DNA sequences required for transformation and selection in plants, an expression construct including the coding sequence of a gene that a operatively linked to a promoter for expressing said DNA sequence; and (b) recovery of a plant which contains the expression construct.

The transgenic plant generated by the methods disclosed above may exhibit an altered trait or stress response. The altered traits may include increased tolerance to extreme temperature, such as heat or cold; or increased tolerance to extreme water condition such as drought or excessive water. The transgenic plant may exhibits one or more altered phenotype that may contribute to the resistance to drought condition. These phenotypes may include, by way of example, early maturation, increased growth rate, increased biomass, or increased lipid content.

In accordance with the disclosed methods, the coding sequence to be introduced in the transgenic plant preferably encodes a peptide having at least 70%, more preferably at least 90%, more preferably at least 98% identity, and even more preferably at least 99% identity to the polypeptide encoded by the DRGs disclosed in this application. In an alternative aspect, DNA sequence may be oriented in an antisense direction relative to said promoter within said construct.

In accordance with the methods of the present invention, the promoter is preferably selected from the group consisting of an constitutive promoter, an inducible promoter, a tissue specific promoter, and organ specific promoter, a cell-specific promoter. More preferably the promoter is an inducible promoter for expressing said DNA sequence under water deficit conditions.

In another aspect, the present invention provides a method of identifying whether a plant that has been successfully transformed with a construct, characterized in that the method comprises the steps of: (a) introducing into plant cells capable of being transformed and regenerated into whole plants a construct comprising, in addition to the DNA sequences required for transformation and selection in plants, an expression construct that includes a DNA sequence selected from at least one of the DRGs disclosed herein, said DNA sequence may be operatively linked to a promoter for expressing said DNA sequence; (b) regenerating the plant cells into whole plants; and (c) subjecting the plants to a screening process to differentiate between transformed plants and non-transformed plants.

The screening process may involve subjecting the plants to environmental conditions suitable to kill non-transformed plants, retain viability in transformed plants. For instance by growing the plants in a medium or soil that contains certain chemicals, such that only those plants expressing the transgenes can survive. In one particular embodiment, after obtaining a transgenic plant that appear to be expressing the transgene, a functional screening may be carried out by growing the plants under water deficit conditions to select for those that can tolerate such a condition.

In another aspect, the present disclosure provides a kit for generating a transgenic plant having an altered stress response or an altered phenotype compared to an unmodified plant, characterized in that the kit comprises: an expression construct including a DNA sequence selected from at least one of the DRGs disclosed herein, said DNA sequence may be operatively linked to an promoter suitable for expressing said DNA sequence in a plant cell.

Preferably the kit further includes targeting means for targeting the activity of the protein expressed from the construct to certain tissues or cells of the plant. Preferably the targeting means comprises an inducible, tissue-specific promoter for specific expression of the DNA sequence within certain tissues of the plant. Alternatively the targeting means may be a signal sequence encoded by said expression construct and may contain a series of amino acids covalently linked to the expressed protein.

In accordance with the kit of the present invention, the DNA sequence may encode a peptide having at least 70%, more preferably at least 90%, more preferably at least 98%, or even 99% identity to the peptide encoded by coding sequences selected from at least one of the DRGs disclosed herein. In one aspect, said DNA sequence may be oriented in an antisense direction relative to said promoter within said construct.

### BRIEF DESCRIPTION OF THE DRAWINGS

FIG. 1 shows the classification of soybean transcription factor families and the number of putative members in each family.

FIG. 2 shows the number of TF genes included in the Soybean transcription factor primer library.

FIG. 3 illustrate the number of soybean tissue specific transcription factors identified through quantitative real time PCR.

FIG. 4 shows some examples of soybean tissue specific genes and their expression pattern across ten soybean tissues.

FIG. 5 shows expression of a bHLH TF gene in mature root cells in a reporter gene system using GUS (β-glucosidase) and GFP (green fluorescent protein) as reporter genes.

FIG. 6 shows gene expression patterns of selected transcription factors which are expressed at specific developmental stages during seed development.

FIG. 7 demonstrates different Soybean transcription factors showing significantly different expression patterns of selected transcription factors across two soybean genotypes, one being flooding resistant, the other being flooding sensitive.

FIG. 8 shows the expression patterns of soybean selected regulatory genes regulated during nodule development. The expression pattern through different stages of nodule development [0 (white bar), 4 (light grey bars), 8 (grey bars), 16 (dark grey bars), 24 (bars with horizontal stripes) and 32 days (black bars) after B. japonicum inoculation and in response to KNO₃ treatment (bars with slanted stripes) were investigated for 16 different soybean regulatory genes

FIG. 9 shows the effects of silencing of 523065855 MYB transcription factor affects soybean nodule development. Standard error bars are shown. P-value <0.04. (A) Comparison of nodule number between RNAi-GUS (grey bar) and RNAi 523065855 soybean roots (white bar). (B) Comparison of nodule size between RNAi-GUS (left) and RNAi 523065855 (right) roots. (C) Gene expression analysis of S23065855 in RNAi-GUS (left) and RNAi S23065855 (right) nodules. (D) Confirmation of the specificity of RNAi construct in the silencing of S23065855.

FIG. 10 shows the expression pattern of a MYB transcription factor during nodulation using GFP (A, B) and GUS (C, D, E, F) as reporter genes.

FIG. 11 shows the expression pattern of selected transcription factors in soybean root nodules.

FIG. 12 summarizes the classification of drought responsive transcripts in soybean leaf tissues based on reported or predicted function of the corresponding proteins.

FIG. 13 summarizes the classification of drought responsive transcripts in soybean root tissues based on reported or predicted function of the corresponding proteins.

FIG. 14 shows the distribution of soybean transcription factor genes expressed specifically in one soybean tissue based on their family membership. Sub-pies highlight the distribution of specific transcription factor gene families in the different tissues based on the specificity of their expression.

FIG. 15 shows the genome database ID numbes of members of the ABI3-vpl family of soybean transcription factors.

FIG. 16 shows the genome database ID numbes of members of the Alfin family of soybean transcription factors.

FIG. 17 shows the genome database ID numbes of members of the AP2-EREBP family of soybean transcription factors.

FIG. 18 shows the genome database ID numbes of members of the ARF family of soybean transcription factors.

FIG. 19 shows the genome database ID numbes of members of the ARID family of soybean transcription factors.

FIG. 20 shows the genome database ID numbes of members of the AS2 family of soybean transcription factors.

FIG. 21 shows the genome database ID numbes of members of the AUX-IAA family of soybean transcription factors.

FIG. 22 shows the genome database ID numbes of members of the BBR-BPC family of soybean transcription factors.

FIG. 23 shows the genome database ID numbes of members of the BES1 family of soybean transcription factors.

FIG. 24 shows the genome database ID numbes of members of the bHLH family of soybean transcription factors.

FIG. 25 shows the genome database ID numbes of members of the bZIP family of soybean transcription factors.

FIG. 26 shows the genome database ID numbes of members of the C2C2-CO like family of soybean transcription factors.

FIG. 27 shows the genome database ID numbes of members of the C2C2-DOF family of soybean transcription factors.

FIG. 28 shows the genome database ID numbes of members of the C2C2-GATA family of soybean transcription factors.

FIG. 29 shows the genome database ID numbes of members of the C2C2-YABBY family of soybean transcription factors.

FIG. 30 shows the genome database ID numbes of members of the C2H2 family of soybean transcription factors.

FIG. 31 shows the genome database ID numbes of members of the C3H family of soybean transcription factors.

FIG. 32 shows the genome database ID numbes of members of the CAMTA family of soybean transcription factors.

FIG. 33 shows the genome database ID numbes of members of the CCAAT-DR1 family of soybean transcription factors.

FIG. 34 shows the genome database ID numbes of members of the CCAAT-HAP2 family of soybean transcription factors.

FIG. 35 shows the genome database ID numbes of members of the CCAAT-HAP3 family of soybean transcription factors.

FIG. 36 shows the genome database ID numbes of members of the CCAAT-HAP5 family of soybean transcription factors.

FIG. 37 shows the genome database ID numbes of members of the CPP family of soybean transcription factors.

FIG. 38 shows the genome database ID numbes of members of the E2F-DP family of soybean transcription factors.

FIG. 39 shows the genome database ID numbes of members of the EIL family of soybean transcription factors.

FIG. 40 shows the genome database ID numbes of members of the FHA family of soybean transcription factors.

FIG. 41 shows the genome database ID numbes of members of the GARP-ARR-B family of soybean transcription factors.

FIG. 42 shows the genome database ID numbes of members of the GARP-G2-like family of soybean transcription factors.

FIG. 43 shows the genome database ID numbes of members of the GeBP family of soybean transcription factors.

FIG. 44 shows the genome database ID numbes of members of the GIF family of soybean transcription factors.

FIG. 45 shows the genome database ID numbes of members of the GRAS family of soybean transcription factors.

FIG. 46 shows the genome database ID numbes of members of the GRF family of soybean transcription factors.

FIG. 47 shows the genome database ID numbes of members of the HB family of soybean transcription factors.

FIG. 48 shows the genome database ID numbes of members of the HMG family of soybean transcription factors.

FIG. 49 shows the genome database ID numbes of members of the HRT-like family of soybean transcription factors.

FIG. 50 shows the genome database ID numbes of members of the HSF family of soybean transcription factors.

FIG. 51 shows the genome database ID numbes of members of the JUMONJI family of soybean transcription factors.

FIG. 52 shows the genome database ID numbes of members of the LFY family of soybean transcription factors.

FIG. 53 shows the genome database ID numbes of members of the LIM family of soybean transcription factors.

FIG. 54 shows the genome database ID numbes of members of the LUG family of soybean transcription factors.

FIG. 55 shows the genome database ID numbes of members of the MADS family of soybean transcription factors.

FIG. 56 shows the genome database ID numbes of members of the MBF1 family of soybean transcription factors.

FIG. 57 shows the genome database ID numbes of members of the MYB family of soybean transcription factors.

FIG. 58 shows the genome database ID numbes of members of the MYB-related family of soybean transcription factors.

FIG. 59 shows the genome database ID numbes of members of the NAC family of soybean transcription factors.

FIG. 60 shows the genome database ID numbes of members of the NIN-like family of soybean transcription factors.

FIG. 61 shows the genome database ID numbes of members of the NZZ family of soybean transcription factors.

FIG. 62 shows the genome database ID numbes of members of the PcG family of soybean transcription factors.

FIG. 63 shows the genome database ID numbes of members of the PHD family of soybean transcription factors.

FIG. 64 shows the genome database ID numbes of members of the PLATZ family of soybean transcription factors.

FIG. 65 shows the genome database ID numbes of members of the S1Fa-like family of soybean transcription factors.

FIG. 66 shows the genome database ID numbes of members of the SAP family of soybean transcription factors.

FIG. 67 shows the genome database ID numbes of members of the SBP family of soybean transcription factors.

FIG. 68 shows the genome database ID numbes of members of the SRS family of soybean transcription factors.

FIG. 69 shows the genome database ID numbes of members of the TAZ family of soybean transcription factors.

FIG. 70 shows the genome database ID numbes of members of the TCP family of soybean transcription factors.

FIG. 71 shows the genome database ID numbes of members of the TLP family of soybean transcription factors.

FIG. 72 shows the genome database ID numbes of members of the Trihelix family of soybean transcription factors.

FIG. 73 shows the genome database ID numbes of members of the ULT family of soybean transcription factors.

FIG. 74 shows the genome database ID numbes of members of the VOZ family of soybean transcription factors.

FIG. 75 shows the genome database ID numbes of members of the Whirly family of soybean transcription factors.

FIG. 76 shows the genome database ID numbes of members of the WRKY family of soybean transcription factors.

FIG. 77 shows the genome database ID numbes of members of the ZD-HD family of soybean transcription factors.

FIG. 78 shows the genome database ID number of members of the ZIM family of soybean transcription factors.

FIG. 79 shows that expression of soybean homeologous genes during nodulation and in response to KNO₃ and KCl treatments.

FIG. 80 shows gene expression patterns of arabidopsis genes involved in the formation and maintenance of the SAM and the determination of flower organs (A) and their putative orthologs in soybean (B). Genevestigator (Hruz et al., 2008) and the soybean gene atlas were mined to establish the expression pattern of the arabidopsis and soybean. genes, respectively.

FIG. 81 shows expression pattern of several related NAC transcription factors under abiotic stress (water, ABA, NaCl and cold stresses).

FIG. 82 shows drought responses of the dehydration inducible GmNAC genes.

FIG. 83 shows transgene expression levels in the independent Arabidopsis transgenic lines. (Q1 is the independent transgenic lines expressing GmNAC3 and Q2 is the independent transgenic lines expressing GmNAC4).

FIG. 84 shows preliminary phenotypic analysis of the transgenic Arabidopsis plants developed using soybean NAC transcription factors.

FIG. 85 shows transgenic Arabidopsis plants with vector control, GmC2H2 and GmDOF27 transcription factors.

### DETAILED DESCRIPTION

The methods and materials described herein relate to gene expression profiling using microarrays, quantitative RT-PCR, or high throughput sequencing methods, and follow-up analysis to decode the regulatory network that controls a plant's response to stress. More particularly, drought response is analyzed at the molecular level to identify genes and/or promoters which may be activated under water deficit conditions. The coding sequences of such genes may be introduced into a host plant to obtain transgenic plants that are more tolerant to drought than unmodified plants.

It is to be understood that the materials and methods are taught by way of example, and not by limitation. The disclosed instrumentalities may be broader than the particular methods and materials described herein, which may vary within the skill of the art. It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the related art. The following terminology and grammatical variants are used in accordance with the definitions set out below.

The present disclosure provides genes whose expression levels are altered in response to stress conditions in soybean plants using genome-wide microarray (or gene chip) analysis of soybean plants grown under water deficit conditions. Those genes identified using microarray analysis may be subject to validation to confirm that their expression levels are altered under the stress conditions. Validation may be conducted using high throughput two-step qRT-PCR or by the delta delta CT method.

Sequences of those genes that have been validated may be subject to further sequence analysis by comparing their sequences to published sequences of various families of genes or proteins. For instance, some of these DRGs may encode proteins with substantial sequence similarity to known transcription factors. These transcription factors may play a role in the stress response by activating the transcription of other genes.

The present disclosure provides a system and a method for expressing a protein that may enhance a host's capability to grow or to survive in an adverse environment characterized by water deficit. Although plants are the most preferred host for purpose of this disclosure, the genetic constructs described herein may be introduced into other eukaryotic organisms, if the traits conferred upon these organisms by the constructs are desirable.

The term “transgenic plant” refers to a host plant into which a gene construct has been introduced. A gene construct, also referred to as a construct, an expression construct, or a DNA construct, generally contains as its components at least a coding sequence and a regulatory sequence. A gene construct typically contains at least on component that is foreign to the host plant. For purpose of this disclosure, all components of a gene construct may be from the host plant, but these components are not arranged in the host in the same manner as they are in the gene construct. A regulatory sequence is a non-coding sequence that typically contribute to the regulation of gene expression, at the transcription or translation levels. It is to be understood that certain segments in the coding sequence may be translated but may be later removed from the functional protein. An example of these segments is the so-called signal peptide, which may facilitate the maturation or localization of the translated protein, but is typically removed once the protein reaches its destination. Examples of a regulatory sequence include but are not limited to a promoter, an enhancer, and certain post-transcriptional regulatory elements.

After its introduction into a host plant, a gene construct may exist separately from the host chromosomes. Preferably, the entire gene construct, or at least part of it, is integrated onto a host chromosome. The integration may be mediated by a recombination event, which may be homologous, or non-homologous recombination. The term “express” or “expression” refers to production of RNAs using DNAs as template through transcription or translation of proteins from RNAs or the combination of both transcription and translation.

A “host cell,” as used herein, refers to a prokaryotic or eukaryotic cell that contains heterologous DNA which has been introduced into the cell by any means, e.g., electroporation, calcium phosphate precipitation, microinjection, transformation, viral infection, and/or the like. A “host plant” is a plant into which a transgene is to be introduced.

A “vector” is a composition for facilitating introduction, replication and/or expression of a selected nucleic acid in a cell. Vectors include, for example, plasmids, cosmids, viruses, yeast artificial chromosomes (YACs), etc. A “vector nucleic acid” is a nucleic acid vector into which heterologous nucleic acid is optionally inserted and which can then be introduced into an appropriate host cell. Vectors preferably have one or more origins of replication, and one or more sites into which the recombinant DNA can be inserted. Vectors often have convenient markers by which cells with vectors can be selected from those without. By way of example, a vector may encode a drug resistance gene to facilitate selection of cells that are transformed with the vector. Common vectors include plasmids, phages and other viruses, and “artificial chromosomes.” “Expression vectors” are vectors that comprise elements that provide for or facilitate transcription of nucleic acids which are cloned into the vectors. Such elements may include, for example, promoters and/or enhancers operably coupled to a nucleic acid of interest.

“Plasmids” generally are designated herein by a lower case “p” preceded and/or followed by capital letters and/or numbers, in accordance with standard nomenclatures that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors are well known and readily available to those of skill in the art. Moreover, those of skill readily may construct any number of other plasmids suitable for use as described below. The properties, construction and use of such plasmids, as well as other vectors, is readily apparent to those of ordinary skill upon reading the present disclosure.

When a molecule is identified in or can be isolated from a organism, it can be said that such a molecule is derived from said organism. When two organisms have significant difference in the genetic materials in their respective genomes, these two organisms can be said to be genetically different. For purpose of this disclosure, the term “plant” means a whole plant, a seed, or any organ or tissue of a plant that may potentially deveolop into a whole plant.

The term “isolated” means that the material is removed from its original environment, such as the native or natural environment if the material is naturally occurring. For example, a naturally-occurring nucleic acid, polypeptide, or cell present in a living animal is not isolated, but the same polynucleotide, polypeptide, or cell separated from some or all of the coexisting materials in the natural system, is isolated, even if subsequently reintroduced into the natural system. Such nucleic acids can be part of a vector and/or such nucleic acids or polypeptides could be part of a composition, and still be isolated in that such vector or composition is not part of its natural environment.

A “recombinant nucleic acid” is one that is made by recombining nucleic acids, e.g., during cloning, DNA evolution or other procedures. A “recombinant polypeptide” is a polypeptide which is produced by expression of a recombinant nucleic acid. An “amino acid sequence” is a polymer of amino acid residues (a protein, polypeptide, etc.) or a character string representing an amino acid polymer, depending on context. Either the given nucleic acid or the complementary nucleic acid can be determined from any specified polynucleotide sequence.

The terms “nucleic acid,” or “polynucleotide” refer to a deoxyribonucleotide, in the case of DNA, or ribonucleotide in the case of RNA polymer in either single- or double-stranded form, and unless otherwise specified, encompasses known analogues of natural nucleotides that can be incorporated into nucleic acids in a manner similar to naturally occurring nucleotides. A “polynucleotide sequence” is a nucleic acid which is a polymer of nucleotides (A,C,T,U,G, etc. or naturally occurring or artificial nucleotide analogues) or a character string representing a nucleic acid, depending on context. Either the given nucleic acid or the complementary nucleic acid can be determined from any specified polynucleotide sequence.

A “subsequence” or “fragment” is any portion of an entire sequence of a DNA, RNA or polypeptide molecule, up to and including the complete sequence. Typically a subsequence or fragment comprises less than the full-length sequence, and is sometimes referred to as the “truncated version.”

Nucleic acids and/or nucleic acid sequences are “homologous” when they are derived, naturally or artificially, from a common ancestral nucleic acid or nucleic acid sequence. Proteins and/or protein sequences are homologous when their encoding DNAs are derived, naturally or artificially, from a common ancestral nucleic acid or nucleic acid sequence. Similarly, nucleic acids and/or nucleic acid sequences are homologous when they are derived, naturally or artificially, from a common ancestral nucleic acid or nucleic acid sequence. The homologous molecules can be termed homologs. For example, any naturally occurring DRGs, as described herein, can be modified by any available mutagenesis method. When expressed, this mutagenized nucleic acid encodes a polypeptide that is homologous to the protein encoded by the original DRGs. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The terms “identical” or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci. U.S.A. 85:2444; by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp (1988) Gene 73:237-244 and Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment.

In one class of embodiments, the polypeptides herein are at least 70%, generally at least 75%, optionally at least 80%, 85%, 90%, 98% or 99% or more identical to a reference polypeptide, e.g., those that are encoded by DNA sequences as set forth by any one of the DRGs disclosed herein or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or more identical to a reference nucleic acid, e.g., those that are set forth by any one of the DRGs disclosed herein or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” as applied to nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “polypeptide” is used interchangeably with the terms “polypeptides” and “protein(s)”, and refers to a polymer of amino acid residues. A ‘mature protein’ is a protein which is full-length and which, optionally, includes glycosylation or other modifications typical for the protein in a given cellular environment.

The term “variant” or “mutant” with respect to a polypeptide refers to an amino acid sequence that is altered by one or more amino acids with respect to a reference sequence. The variant may have “conservative” changes, wherein a substituted amino acid has similar structural or chemical properties, e.g., replacement of leucine with isoleucine. Alternatively, a variant may have “nonconservative” changes, e.g., replacement of a glycine with a tryptophan. Analogous minor variation can also include amino acid deletion or insertion, or both. Guidance in determining which amino acid residues can be substituted, inserted, or deleted without eliminating biological or immunological activity can be found using computer programs well known in the art, for example, DNASTAR software.

A variety of additional terms are defined or otherwise characterized herein. In practicing the instrumentalities described herein, many conventional techniques in molecular biology, microbiology, and recombinant DNA are optionally used. These techniques are well known to those of ordinary skill in the art. For example, one skilled in the art would be familiar with techniques for in vitro amplification methods, including the polymerase chain reaction (PCR), for the production of the homologous nucleic acids described herein.

In addition, commercially available kits may facilitate the purification of plasmids or other relevant nucleic acids from cells. See, for example, EasyPrep™ and FlexiPrep™ kits, both from Pharmacia Biotech; StrataClean™ from Stratagene; and, QIAprep™ from Qiagen. Any isolated and/or purified nucleic acid can be further manipulated to produce other nucleic acids, used to transfect cells, incorporated into related vectors to infect organisms, or the like. Typical cloning vectors contain transcription terminators, transcription initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both.

Various types of mutagenesis are optionally used to modify DRGs and their encoded polypeptides, as described herein, to produce conservative or non-conservative variants. Any available mutagenesis procedure can be used. Such mutagenesis procedures optionally include selection of mutant nucleic acids and polypeptides for one or more activity of interest. Procedures that can be used include, but are not limited to: site-directed point mutagenesis, random point mutagenesis, in vitro or in vivo homologous recombination (DNA shuffling), mutagenesis using uracil-containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, double-strand break repair, mutagenesis by chimeric constructs, and many others known to persons of skill in the art.

In one embodiment, mutagenesis can be guided by known information about the naturally occurring molecule or altered or mutated naturally occurring molecule. By way of example, this known information may include sequence, sequence comparisons, physical properties, crystal structure and the like. In another class of mutagenesis, modification is essentially random, e.g., as in classical DNA shuffling.

Polypeptides may include variants, in which the amino acid sequence has at least 70% identity, preferably at least 80% identity, typically 90% identity, preferably at least 95% identity, more preferably at least 98% identity and most preferably at least 99% identity, to the amino acid sequences as encoded by the DNA sequences set forth in any one of the DRGs disclosed herein.

The aforementioned polypeptides may be obtained by any of a variety of methods. Smaller peptides (less than 50 amino acids long) are conveniently synthesized by standard chemical techniques and can be chemically or enzymatically ligated to form larger polypeptides. Polypeptides can be purified from biological sources by methods well known in the art, for example, as described in Protein Purification, Principles and Practice, Second Edition Scopes, Springer Verlag, N.Y. (1987) Polypeptides are optionally but preferably produced in their naturally occurring, truncated, or fusion protein forms by recombinant DNA technology using techniques well known in the art. These methods include, for example, in vitro recombinant DNA techniques, synthetic techniques and in vivo genetic recombination. See, for example, the techniques described in Sambrook et al. (2001) Molecular Cloning, A Laboratory Manual, Third Edition, Cold Spring Harbor Press, N.Y.; and Ausubel et al., eds. (1997) Current Protocols in Molecular Biology, Green Publishing Associates, Inc., and John Wiley & Sons, Inc., N.Y (supplemented through 2002). RNA encoding the proteins may also be chemically synthesized. See, for example, the techniques described in Oligonucleotide Synthesis, (1984) Gait ed., IRL Press, Oxford, which is incorporated by reference herein in its entirety.

The nucleic acid molecules described herein may be expressed in a suitable host cell or an organism to produce proteins. Expression may be achieved by placing a nucleotide sequence encoding these proteins into an appropriate expression vector and introducing the expression vector into a suitable host cell, culturing the transformed host cell under conditions suitable for expression of the proteins described or variants thereof, or a polypeptide that comprises one or more domains of such proteins. The recombinant proteins from the host cell may be purified to obtain purified and, preferably, active protein. Alternatively, the expressed protein may be allowed to function in the intact host cell or host organism.

Appropriate expression vectors are known in the art, and may be purchased or applied for use according to the manufacturer's instructions to incorporate suitable genetic modifications. For example, pET-14b, pcDNAlAmp, and pVL1392 are available from Novagen and Invitrogen, and are suitable vectors for expression in E. coli, mammalian cells and insect cells, respectively. These vectors are illustrative of those that are known in the art, and many other vectors can be used for the same purposes. Suitable host cells can be any cell capable of growth in a suitable media and allowing purification of the expressed protein. Examples of suitable host cells include bacterial cells, such as E. coli, Streptococci, Staphylococci, Streptomyces and Bacillus subtilis cells; fungal cells such as Saccharomyces and Aspergillus cells; insect cells such as Drosophila S2 and Spodoptera Sf9 cells, mammalian cells such as CHO, COS, HeLa, 293 cells; and plant cells.

Culturing and growth of the transformed host cells can occur under conditions that are known in the art. The conditions will generally depend upon the host cell and the type of vector used. Suitable culturing conditions may be used such as temperature and chemicals and will depend on the type of promoter utilized.

Purification of the proteins or domains of such proteins, if desired, may be accomplished using known techniques without performing undue experimentation. Generally, the transformed cells expressing one of these proteins are broken, crude purification occurs to remove debris and some contaminating proteins, followed by chromatography to further purify the protein to the desired level of purity. Host cells may be broken by known techniques such as homogenization, sonication, detergent lysis and freeze-thaw techniques. Crude purification can occur using ammonium sulfate precipitation, centrifugation or other known techniques. Suitable chromatography includes anion exchange, cation exchange, high performance liquid chromatography (HPLC), gel filtration, affinity chromatography, hydrophobic interaction chromatography, etc. Well known techniques for refolding proteins can be used to obtain the active conformation of the protein when the protein is denatured during intracellular synthesis, isolation or purification.

In general, DRG proteins or domains, or antibodies to such proteins can be purified, either partially (e.g., achieving a 5×, 10×, 100×, 500×, or 1000× or greater purification), or even substantially to homogeneity (e.g., where the protein is the main component of a solution, typically excluding the solvent (e.g., water or DMSO) and buffer components (e.g., salts and stabilizers) that the protein is suspended in, e.g., if the protein is in a liquid phase), according to standard procedures known to and used by those of skill in the art. Accordingly, the polypeptides can be recovered and purified by any of a number of methods well known in the art, including, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. In one embodiment, antibodies made against the proteins described herein are used as purification reagents, e.g., for affinity-based purification of proteins comprising one or more DRG protein domains or antibodies thereto. Once purified, partially or to homogeneity, as desired, the polypeptides are optionally used e.g., as assay components, therapeutic reagents or as immunogens for antibody production.

In addition to other references noted herein, a variety of purification methods are well known in the art, including, for example, those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana, Bioseparation of Proteins, Academic Press, Inc. (1997); Bollag et al., Protein Methods, 2nd Edition Wiley-Liss, NY; Walker (1996) The Protein Protocols Handbook Humana Press, NJ; Harris and Angal Protein Purification Applications: A Practical Approach IRL Press at Oxford, Oxford, England (1990); Scopes, Protein Purification: Principles and Practice 3rd Edition Springer Verlag, NY (1993); Janson and Ryden, Protein Purification: Principles, High Resolution Methods and Applications, Second Edition Wiley-VCH, NY (1998); and Walker, Protein Protocols on CD-ROM Humana Press, NJ (1998); and the references cited therein.

After synthesis, expression and/or purification, proteins may possess a confoimation different from the desired conformations of the relevant polypeptides. For example, polypeptides produced by prokaryotic systems often are optimized by exposure to chaotropic agents to achieve proper folding. During purification from, e.g., lysates derived from E. coli, the expressed protein is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art. Debinski, et al., for example, describe the denaturation and reduction of inclusion body proteins in guanidine-DTE. The proteins can be refolded in a redox buffer containing, e.g., oxidized glutathione and L-arginine. Refolding reagents can be flowed or otherwise moved into contact with the one or more polypeptide or other expression product, or vice-versa.

In another aspect, antibodies to the DRG proteins or fragments thereof may be generated using methods that are well known in the art. The antibodies may be utilized for detecting and/or purifying the DRG proteins, optionally discriminating the proteins from various homologues. As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein.

General protocols that may be adapted for detecting and measuring the expression of the described DRG proteins using the above mentioned antibodies are known. Such methods include, but are not limited to, dot blotting, western blotting, competitive and noncompetitive protein binding assays, enzyme-linked immunosorbant assays (ELISA), immunohistochemistry, fluorescence-activated cell sorting (FACS), and other protocols that are commonly used and widely described in scientific and patent literature.

Sequence of the DRG genes may also be used in genetic mapping of plants or in plant breeding. Polynucleotides derived from the DRG gene sequences may be used in in situ hybridization to determine the chromosomal locus of the DRG genes on the chromosomes. These polynucleotides may also be used to detect segregation of different alleles at certain DRG loci.

Sequence information of the DRG genes may also be used to design oligonucleotides for detecting DRG mRNA levels in the cells or in plant tissues. For example, the oligonucleotides can be used in a Northern blot analysis to quantify the levels of DRG mRNA. Moreover, full-length or fragment of the DRG genes may be used in preparing microarrays (or gene chips). Full-length or fragment of the DRG genes may also be used in microarray experiments to study expression profile of the DRG genes. High-throughput screening can be conducted to measure expression levels of the DRG genes in different cells or tissues. Various compounds or other external factors may be screened for their effects expression of the DRG gene expression.

Sequences of the DRG genes and proteins may also provide a tool for identification of other proteins that may be involved in plant drought response. For example, chimeric DRG proteins can be used as a “bait” to identify other proteins that interact with DRG proteins in a yeast two-hybrid screening. Recombinant DRG proteins can also be used in pull-down experiment to identify their interacting proteins. These other proteins may be cofactors that enhance the function of the DRG proteins, or they may be DRG proteins themselves which have not been identified in the experiments disclosed herein.

The DRG polypeptides may possess structural features which can be recognized, for example, by using immunological assays. The generation of antisera which specifically bind the DRG polypeptides, as well as the polypeptides which are bound by such antisera, are a feature of the disclosed embodiments.

In order to produce antisera for use in an immunoassay, one or more of the immunogenic DRG polypeptides or fragments thereof are produced and purified as described herein. For example, recombinant protein may be produced in a host cell such as a bacterial or an insect cell. The resultant proteins can be used to immunize a host organism in combination with a standard adjuvant, such as Freund's adjuvant. Commonly used host organisms include rabbits, mice, rats, donkeys, chickens, goats, horses, etc. An inbred strain of mice may also be used to obtain more reproducible results due to the virtual genetic identity of the mice. The mice are immunized with the immunogenic DRG polypeptides in combination with a standard adjuvant, such as Freund's adjuvant, and a standard mouse immunization protocol. See, for example, Harlow and Lane, Antibodies, A Laboratory Manual, Cold Spring Harbor Publications, New York (1988), which provides comprehensive descriptions of antibody generation, immunoassay formats and conditions that can be used to determine specific immunoreactivity. Alternatively, one or more synthetic or recombinant DRG polypeptides or fragments thereof derived from the sequences disclosed herein is conjugated to a carrier protein and used as an immunogen.

Antisera that specifically bind the DRG proteins may be used in a range of applications, including but not limited to immunofluorescence staining of cells for the expression level and localization of the DRG proteins, cytological staining for the expression of DRG proteins in tissues, as well as in Western blot analysis.

Another aspect of the disclosure includes screening for potential or candidate modulators of DRG protein activity. For example, potential modulators may include small molecules, organic molecules, inorganic molecules, proteins, hormones, transcription factors, or the like, which can be contacted to a cell or certain tissues that express the DRG proteins to assess the effects, if any, of the candidate modulator upon DRG protein activity.

Alternatively, candidate modulators may be screened to modulate expression of DRG proteins. For example, potential modulators may include small molecules, organic molecules, inorganic molecules, proteins, hormones, transcription factors, or the like, which can be contacted to a cell or certain tissues that express the DRG proteins, to assess the effects, if any, of the candidate modulator upon DRG protein expression. Expression of a DRG gene described herein may be detected, for example, via Northern blot analysis or quantitative (optionally real time) RT-PCR, before and after application of potential expression modulators. Alternatively, promoter regions of the various DRG genes may be coupled to reporter constructs including, without limitation, CAT, beta-galactosidase, luciferase or any other available reporter, and may similarly be tested for expression activity modulation by the candidate modulator. Promoter regions of the various genes are generally sequences in the proximity upstream of the start site of transcription, typically within 1 Kb or less of the start site, such as within 500 bp, 250 by or 100 by of the start site. In certain cases, a promoter region may be located between 1 and 5 Kb from the start site.

In either case, whether the assay is to detect modulated activity or expression, a plurality of assays may be performed in a high-throughput fashion, for example, using automated fluid handling and/or detection systems in serial or parallel fashion. Similarly, candidate modulators can be tested by contacting a potential modulator to an appropriate cell using any of the activity detection methods herein, regardless of whether the activity that is detected is the result of activity modulation, expression modulation or both.

A method of modifying a plant may include introducing into a host plant one or more DRG genes described above. The DRG genes may be placed in an expression construct, which may be designed such that the DRG protein(s) are expressed constitutively, or inducibly. The construct may also be designed such that the DRG protein(s) are expressed in certain tissue(s), but not in other tissue(s). The DRG protein(s) may enhance the ability of the host plant in drought tolerance, such as by reducing water loss or by other mechanisms that help a plant cope with water deficit growth conditions. The host plant may include any plants whose growth and/or yield may be enhanced by a modified drought response. Methods for generating such transgenic plants is well known in the field. See e.g., Leandro Peña (Editor), Transgenic Plants: Methods and Protocols (Methods in Molecular Biology), Humana Press, 2004.

The use of gene inhibition technologies such as antisense RNA or co-suppression or double stranded RNA interference is also within the scope of the present disclosure. In these approaches, the isolated gene sequence is operably linked to a suitable regulatory element. In one embodiment of the disclosure, the construct contains a DNA expression cassette that contains, in addition to the DNA sequences required for transformation and selection in said cells, a DNA sequence that encodes a DRG proteins or a DRG modulator protein, with at least a portion of said DNA sequence in an antisense orientation relative to the normal presentation to the transcriptional regulatory region, operably linked to a suitable transcriptional regulatory region such that said recombinant DNA construct expresses an antisense RNA or portion thereof of an antisense RNA in the resultant transgenic plant.

It is apparent to one of skill in the art that the polynucleotide encoding the DRG proteins or a DRG modulator proteins can be in the antisense (for inhibition by antisense RNA) or sense (for inhibition by co-suppression) orientation, relative to the transcriptional regulatory region. Alternatively a combination of sense and antisense RNA expression can be utilized to induce double stranded RNA interference. See, e.g., Chuang and Meyerowitz, PNAS 97: 4985-4990, 2000; also Smith et al., Nature 407: 319-320, 2000.

These methods for generation of transgenic plants generally entail the use of transformation techniques to introduce the gene or construct encoding the DRG proteins or a DRG modulator proteins, or a part or a homolog thereof, into plant cells. Transfoimation of a plant cell can be accomplished by a variety of different methodology. Methods that have general utility include, for example, Agrobacterium based systems, using either binary and/or cointegrate plasmids of both A. tumifaciens and A. rhyzogenies, (See e.g., U.S. Pat. No. 4,940,838, U.S. Pat. No. 5,464,763), the biolistic approach (See e.g, U.S. Pat. No. 4,945,050, U.S. Pat. No. 5,015,580, U.S. Pat. No. 5,149,655), microinjection, (See e.g., U.S. Pat. No. 4,743,548), direct DNA uptake by protoplasts, (See e.g., U.S. Pat. No. 5,231,019, U.S. Pat. No. 5,453,367) or needle-like whiskers (See e.g., U.S. Pat. No. 5,302,523). Any method for the introduction of foreign DNA into a plant cell and for expression therein may be used within the context of the present disclosure.

Plants that are capable of being transformed encompass a wide range of species, including but not limited to soybean, corn, potato, rice, wheat and many other crops, fruit plants, vegetables and tobacco. See generally, Vain, P., Thirty years of plant transformation technology development, Plant Biotechnol J. 2007 March; 5(2):221-9. Any plants that are capable of taking in foreign DNA and transcribing the DNA into RNA and/or further translating the RNA into a protein may be a suitable host.

The modulators described above that may alter the expression levels or the activity of the DRG proteins (collectively called DRG modulators) may also be introduced into a host plant in the same or similar manner as described above.

The DRG proteins or the DRG modulators may be used to modify a target plant by causing them to be assimilated by the plant. Alternatively, the DRG proteins or the DRG modulators may be applied to a target plant by causing them to be in contact with the plant, or with a specific organ or tissue of the plant. In one embodiment, organic or inorganic molecules that can function as DRG modulators may be caused to be in contact with a plant such that these chemicals may enhance the drought response of the target plant.

In addition to the DRG modulators, DRG polypeptides or DRG nucleic acids, a composition containing other ingredients may be introduced, administered or delivered to the plant to be modified. In one aspect, a composition containing an agriculturally acceptable ingredient may be used in conjunction with the DRG modulators to be administered or delivered to the plant.

Bioinformatic systems are widely used in the art, and can be utilized to identify homology or similarity between different character strings, or can be used to perform other desirable functions such as to control output files, provide the basis for making presentations of information including the sequences and the like. Examples include BLAST, discussed supra. For example, commercially available databases, computers, computer readable media and systems may contain character strings corresponding to the sequence information herein for the DRG polypeptides and nucleic acids described herein. These sequences may include specifically the DRG sequences listed herein and the various silent substitutions and conservative substitutions thereof.

The bioinformatic systems contain a wide variety of information that includes, for example, a complete sequence listings for the entire genome of an individual organism representing a species. Thus, for example, using the DRG sequences as a basis for comparison, the bioinformatic systems may be used to compare different types of homology and similarity of various stringency and length on the basis of reported data. These comparisons are useful to identify homologs or orthologs where, for example, the basic DRG gene ortholog is shown to be conserved across different organisms. Thus, the bioinformatic systems may be used to detect or recognize the homologs or orthologs, and to predict the function of recognized homologs or orthologs. By way of example, many homology determination methods have been designed for comparative analysis of sequences of biopolymers including nucleic acids, proteins, etc. With an understanding of hydrogen bonding between the principal bases in natural polynucleotides, models that simulate annealing of complementary homologous polynucleotide strings can also be used as a foundation of sequence alignment or other operations typically performed on the character strings corresponding to the sequences herein. One example of a software package for calculating sequence similarity is BLAST, which can be adapted to the present invention by inputting character strings corresponding to the sequences herein.

The software can also include output elements for controlling nucleic acid synthesis (e.g., based upon a sequence or an alignment of a sequences herein) or other operations which occur downstream from an alignment or other operation performed using a character string corresponding to a sequence herein.

In an additional aspect, kits may embody any of the methods, compositions, systems or apparatus described above. Kits may optionally comprise one or more of the following: (1) a composition, system, or system component as described herein; (2) instructions for practicing the methods described herein, and/or for using the compositions or operating the system or system components herein; (3) a container for holding components or compositions, and, (4) packaging materials.

### EXAMPLES

The nonlimiting examples that follow report general procedures, reagents and characterization methods that teach by way of example, and should not be construed in a narrowing manner that limits the disclosure to what is specifically disclosed. Those skilled in the art will understand that numerous modifications may be made and still the result will fall within the spirit and scope of the present invention.

### Example 1

### Classification of Regulatory Genes in the Soybean Genome

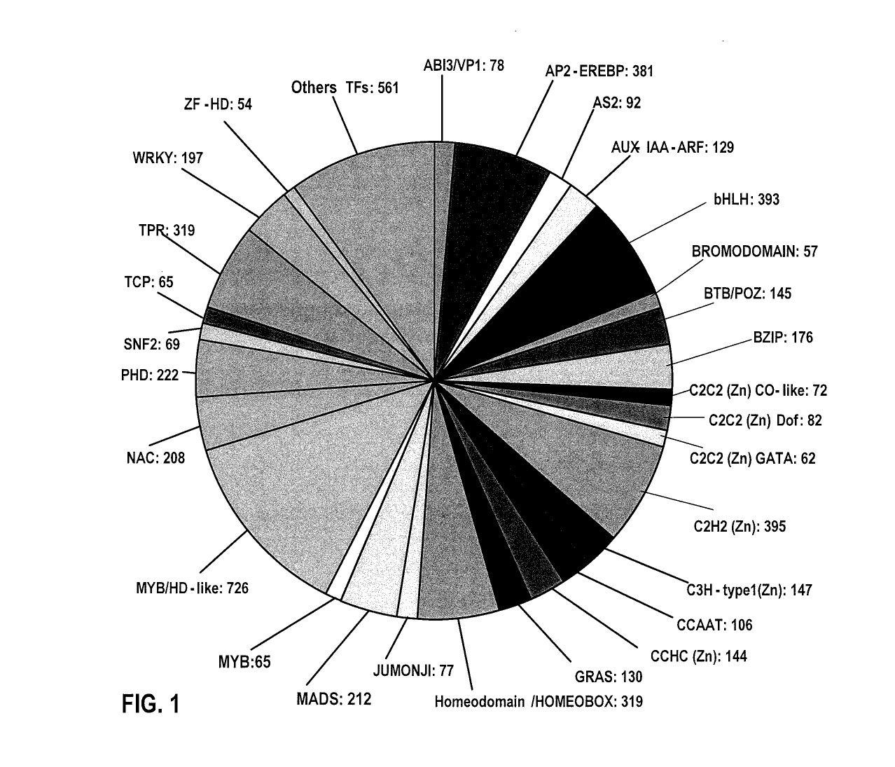

The soybean genome has been sequenced by the Department of Energy-Joint Genome Institute (DOE-JGI) and is publicly available. Mining of this sequence identified 5671 soybean genes as putative regulatory genes, including transcription factors. These genes were comprehensively annotated based on their domain structures. (FIG. 1).

To provide easy access to all soybean TF genes, SoyDB—a central knowledge database has been developed for all the transcription factors in the soybean genome. The database contains protein sequences, predicted tertiary structures, DNA binding sites, domains, homologous templates in the Protein Data Bank (Berman 2000) (PDB), protein family classifications, multiple sequence alignments, consensus DNA binding motifs, web logo of each family, and web links to general protein databases including SwissProt (Boeckmann et al. 2003), Gene Ontology (Ashburner et al 2000), KEGG (Kanehisa et al. 2008), EMBL (Angiuoli et al. 2008), TAIR (Rhee et al. 2003), InterPro (Mulder et al. 2002), SMART (Letunic et al. 2006), PROSITE (Hulo et al. 2006), NCBI, and Pfam (Bateman et al. 2004). The database can be accessed through an interactive and convenient web server, which supports full-text search, PSI-BLAST sequence search, database browsing by protein family, and automatic classification of a new protein sequence into one of 64 annotated transcription factor families by hidden Markov model. Major groups of these families are shown in FIG. 1.

The database schema were implemented in MySQL, together with web-based database access scripts. The scripts automatically execute bioinformatics tools, parse results, create a MySQL database, generated PHP web scripts, and search other protein databases. The fully automated approach can be easily used to create protein annotation databases for any species.

Several bioinformatics tools were used to generate annotations of the soybean transcription factors. An accurate protein structure prediction tool MULTICOM (Cheng 2008) was also used to predict the tertiary structure of each transcription factor when homologous template structures could be found in the PDB. According to the official evaluations during the 8th community-wide Critical Assessment of Techniques for Protein Structure Prediction (CASP8) (http://predictioncenter.org/casp8/), MULTICOM was able to predict with high accuracy three dimensional structures with an average GDT-TS score 0.87 if suitable templates can be found. GDT-TS score ranges from 0 to 1 measuring the similarities of the predicted and real structures, while 1 indicates completely the same and 0 completely different. In SoyDB, the predicted tertiary structure is visualized by Jmol Zemla 2003). Users can view the structures from various perspectives in a three dimensional way.

The predicted structure was parsed into domains by Protein Domain Parser (PDP) (Hughes and Krough 1995). Since a few transcription factors did not have homologous templates in the PDB, DOMAC (Cheng 2007), an accurate ab initio domain prediction tool, was also used to predict the domains for each protein. During the structure prediction process, MULTICOM also generates the sequence alignments between the transcription factor and its homologous templates using PSI-BLAST.

The protein sequences in the same family were aligned into a multiple sequence alignment by MUSCLE (Edgar 2004). A consensus sequence was derived from the multiple sequence alignment. The multiple alignments were also used to identify the conserved signatures (DNA binding sites) for each family. The conserved binding sites were visualized by WebLogo (Crooks et al. 2004).

In order to annotate the functions of soybean transcription factors, each protein sequence was searched against other protein databases by PSI-BLAST periodically. The other databases include Swiss-port, TAIR, RefSeq, SMART, Pfam, KEGG, SPRINTS, EMBL, InterPro, PROSITE, and Gene Ontology. Web links to other databases were created at SoyDB when the same transcription factor or its homologous protein was found in other databases. For almost every transcription factor, several links to the outsides databases were created, which greatly expanded the annotations. For example, the expanded annotations include: protein features in Swiss-Prot, protein function in Gene Ontology, pathways in KEGG, function sites in PROSITE, and so on.

The comprehensive collection and analyses in SoyDB allows us to perform comparison of TF family distribution across the plant kingdom. The large number of soybean TF genes (5671) described in this study is likely due to the two soybean whole genome duplication events that are known to have occurred, one estimated at 40-50 million years ago (mya) and the most recent approximately 10-15 million years ago (Schlueter, J., et al., Gene duplication and paleopolyploidy in soybean and the implications for whole genome sequencing. BMC genomics, 2007. 8(1): p. 330; and Schlueter, J., et al., Mining EST databases to resolve evolutionary events in major crop species. Genome, 2004. 47(5): p. 868-876.) By comparing the total number of genes in different organisms, it was found that the increase of plant gene number is related to multicellularity and ploidy. For example, compared to the unicellular eukaryote Chlamydomonas reinhardtii where 15,143 genes are predicted (Merchant, S., et al., The Chlamydomonas Genome Reveals the Evolution of Key Animal and Plant Functions. Science, 2007. 318(5848): p. 245), larger numbers of protein-encoding genes are reported in multicellular plant organisms [e.g. Physcomitrella patens (35,938; See Rensing, S., et al., The Physcomitrella Genome Reveals Evolutionary Insights into the Conquest of Land by Plants. Science, 2008. 319(5859): p. 64), Arabidopsis thaliana (32,944; TAIR, http://www.arabidopsis.org/)] and the tetraploid Glycine max [(66,153, Phytozome, http://www.phytozome.net/soybean).

It is hypothesized that TF gene number also follows the same trend as land plants, which have a larger number of TF genes compared to algae. To perform the most complete and current comparisons of plant TF genes and their distributions across TF gene families, we mined the last updated DBD database [9] in eleven plant species (C. reinhardtii, P. patens, Oryza sativa, Zea mays, Sorghum bicolor, Lotus japonicum, Medicago truncatula, A. thaliana, Vinis vinifera, Ricinus communis, and Populus trichocarpa). These species were then compared with the soybean TF genes stored in our SoyDB database.

Our analysis shows that the unicellular C. reinhardtii has the lowest number of TF genes when compared to multicellular land plants (the exceptions are L. japonicus and M. truncatula where only a partial genome sequence is available). This trend also reflects the differences of total gene number in the organisms. For example, it is interesting to note that homeobox, MYB, NAC, and WRKY TF genes in C. reinhardtii lack or have very low representations compared to the eleven other plant models. Previous studies defined a role for homeobox and WRKY genes in plant organ and plant cell development. Therefore, the occurrence of these genes only in multicellular plants may reflect their special roles in development. In addition, a close relationship between TF gene number and total gene number is observed when comparing the TF gene numbers of G. max and A. thaliana with their total gene numbers (i.e. G. max encodes 66,153 protein-coding genes including 5,683 TF genes; A. thaliana encodes 32,944 protein-coding genes and 1,738 TF genes). Thus, the family distribution of soybean TF genes is similar to other land plant species, except for P. patens (e.g. AP2 represents 7% of total TF genes in soybean vs. 8-12% for other land plants; bZIP: 3% vs. 3-7%; bHLH: 7% vs. 8-11%; homeobox: 6% vs. 4-7%; MYB: 14% vs. 7-14%; NAC: 4% vs. 4-9%; WRKY: 3% vs. 4-7%; ZF-C2H2: 7% vs. 5-9%).

### Example 2

### A Primer Library for PCR Amplification of Genes Encoding Soybean Transcription Factors

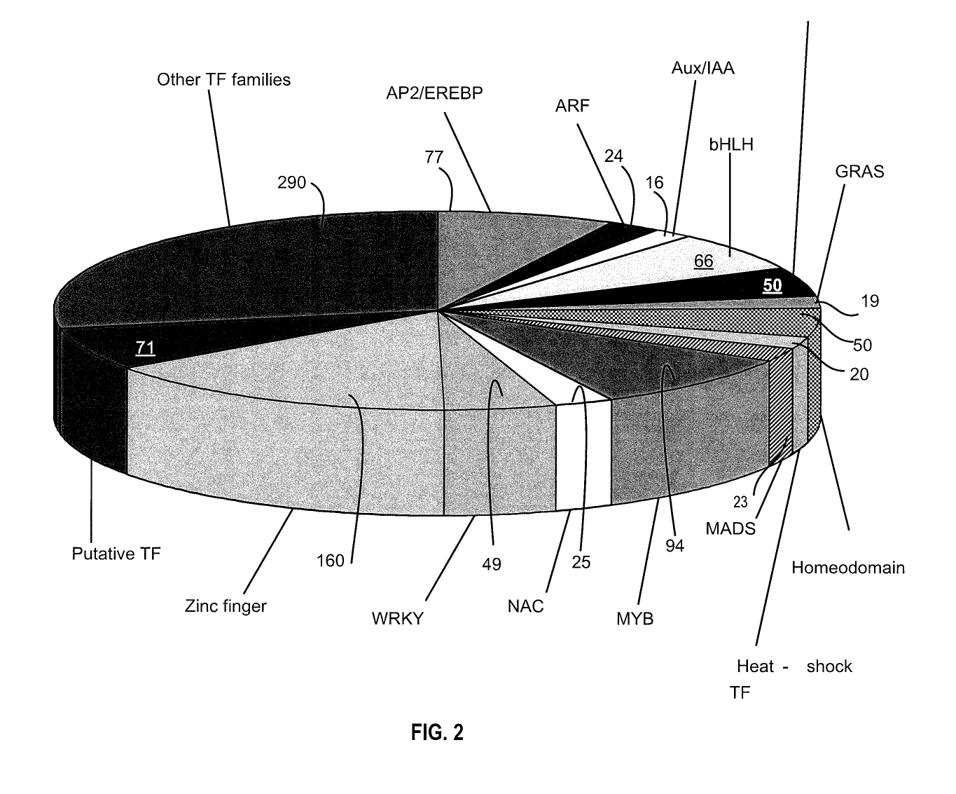

In order to quantitate the expression of TF genes in soybean, a library containing 1149 sets (or pairs) of PCR primer was designed and synthesized. The sequences of these primers and the Identifier of the corresponding gene are listed in Table 1. These primers allowed for sensitive measurement of the expression levels of 1034 different soybean transcription factors (20% of total TF soybean genes). The number and classification of these TF genes are shown in FIG. 2.

TABLE 1
List of primers and sequences in the primer library

| Forward primer | Reverse primer | ID number | Soybean gene ID |
|---|---|---|---|
| CTGCTGCTGATGATGTTCGT (SEQ ID = 1) | ACCACGAACTGCGAGATACC (SEQ ID = 2) | S4898534 | Glyma17g34990 |
| TTTGCAACTGGAGAACGATG (SEQ ID = 3) | ATGAGTATTGGGCCTGACGA (SEQ ID = 4) | S4915781 | Glyma14g29160 |
| TCACACACTCACATTCCGGT (SEQ ID = 5) | GGTCCTTAAGTCATCAGCGG (SEQ ID = 6) | S4901877 | Glyma19g37780 |
| CAGCAGTCAGCAGCAGAATC (SEQ ID = 7) | GGAATTCCACAAGGGATTGA (SEQ ID = 8) | S5096279 | Glyma01g02760 |
| TCACCCTCTTCCTCATCGTC (SEQ ID = 9) | TTGTTGTTGTCTCTCGCTCG (SEQ ID = 10) | TC211213 | Glyma01g35010 |
| CCCCTATTTGTTTTGTGAGCA (SEQ ID = 11) | CAGTTATGTATGGGCTTTTCCT (SEQ ID = 12) | S4911482 | Glyma01g39520 |
| GAGAGAAACAACAGCAGCGA (SEQ ID = 13) | ACTTGCCCCACTTCCTCATC (SEQ ID = 14) | S4969502 | Glyma01g39540 |
| AACATCACTTGGCCTCAACC (SEQ ID = 15) | GTTCGGACTGTGAGTGGGAT (SEQ ID = 16) | CD404474 | Glyma01g39540 |
| CCATTCTGATTGGCTTCTGC (SEQ ID = 17) | GCGGAAAAGAGAGATGGATG (SEQ ID = 18) | S5142323 | Glyma01g40380 |
| TCAATCTAGTCGAAAGCCGTC (SEQ ID = 19) | TTCCGCGTTTGGATTACTCT (SEQ ID = 20) | BE023264 | Glyma01g41530 |
| CACTTTCCACGACCACAATG (SEQ ID = 21) | GAAGCACGAGTAGTGTTCTCTCT (SEQ ID = 22) | AI443715 | Glyma01g41550 |
| CGTACGCGTCAAATTGAGAA (SEQ ID = 23) | AGCCTTTGATGTCTCCTCCA (SEQ ID = 24) | S4991587 | Glyma01g42500 |
| CCCCTAGGTCTTCCAACACA (SEQ ID = 25) | CTCCTTAGGACGCAAAATGG (SEQ ID = 26) | S21567471 | Glyma02g00870 |
| CCAACACCATCTCAAAATCG (SEQ ID = 27) | AAGTGCTTATTTGGCCATGTG (SEQ ID = 28) | CF808401 | Glyma02g07310 |
| GAGACTCATCTTCAGCGACAG (SEQ ID = 29) | GGTGGGGTTTCAGTAACCGT (SEQ ID = 30) | S19677224 | Glyma02g08840 |
| CAGAGGTGCATTAGCCCTTC (SEQ ID = 31) | CATCACAATTGATGGATGGC (SEQ ID = 32) | BI468684 | Glyma02g09600 |
| GATCAACACCACCACCACAA (SEQ ID = 33) | GAAGGGACTCACCGTTGCTA (SEQ ID = 34) | S4892093 | Glyma02g46340 |
| AGGCATCCTCCTTCACCTTT (SEQ ID = 35) | GAAGTCCTAGAAGCGCCAAG (SEQ ID = 36) | BG043825 | Glyma03g26780 |
| TCTCTGCCTCTTCTTGCACTC (SEQ ID = 37) | ATGCACCAAAGAACACACCA (SEQ ID = 38) | S23071305 | Glyma03g27050 |
| TCCAGTTGTATTGGTAGCGTTG (SEQ ID = 39) | ATGGTGGTGGTGGTCGTACT (SEQ ID = 40) | BQ080756 | Glyma03g31940 |
| TTATGTGTATGCTGGAGCGG (SEQ ID = 41) | ACAACACACAACCGACCTGA (SEQ ID = 42) | S5100664 | Glyma04g04350 |
| TGCTTTCCAAAGAAGGAAGC (SEQ ID = 43) | CTCCCTCTCCTCCTTGGTCT (SEQ ID = 44) | S15854043 | Glyma04g08900 |
| TCAACCCCTTCTCCTTCAAA (SEQ ID = 45) | TTTTGGGTGGTGTTGGGTAT (SEQ ID = 46) | TC225042 | Glyma04g11290 |
| CTGTAACATGGTTTTGGGAGT (SEQ ID = 47) | TGCTGTAACCCATGATCAGC (SEQ ID = 48) | S21539774 | Glyma05g18170 |
| CAGCGGTTTCAAATGTTCCT (SEQ ID = 49) | GAGGAGTGAGACAGAGGCCA (SEQ ID = 50) | S5100428 | Glyma05g32040 |
| TTTGGGTTTTACGAGTTGGC (SEQ ID = 51) | TGGTGCCTGTCTCAATCAAA (SEQ ID = 52) | BU965378 | Glyma05g37120 |
| CTTTGTGGTGACTCCGTTGA (SEQ ID = 53) | CTCCAACTGGGTCATGAGGT (SEQ ID = 54) | S5090687 | Glyma06g07240 |
| TTAAGCCTTGTCGATTTCCG (SEQ ID = 55) | GCCACGAATGCGTTTTATCT (SEQ ID = 56) | TC208898 | Glyma06g08990 |
| CACGTCAGCAAACGTCAGAT (SEQ ID = 57) | GGTTGTTTCCGACAAGGAGA (SEQ ID = 58) | S23065007; | Glyma06g11010 |
| | | TC225047 | |
| GGTTGTCTGAACCGGTCAAT (SEQ ID = 59) | GCAACGATGACCAAACTACAA (SEQ ID = 60) | S4875747 | Glyma06g35710 |
| AGCTCTCTTTTGGGCTGACA (SEQ ID = 61) | CCCACTTCATGACCCAGTCT (SEQ ID = 62) | BM527363 | Glyma06g44430 |
| GCAGCCCAAAGAGACTCAAT (SEQ ID = 63) | TCCTTCCTTCTGCTTCCTTTT (SEQ ID = 64) | S4882660 | Glyma06g44430 |
| CATGCTCTCATGACTTGG (SEQ ID = 65) | TGTGAAGAGACACAAAGAGAGT (SEQ ID = 66) | S4877810 | Glyma07g06080 |
| TCCAGCAAAATCCATCATCA (SEQ ID = 67) | GATTCATTCGGGAACAAGGA (SEQ ID = 68) | S4874772 | Glyma07g33510 |
| TTGTCGTACACAATGGCAGC (SEQ ID = 69) | GCGGAGATAAGAGACCCGT (SEQ ID = 70) | S21539521 | Glyma08g02460 |
| TGGAGTCACGGCATTTATGA (SEQ ID = 71) | ACCCTCGAAGCCACAAAGTA (SEQ ID = 72) | S5078767 | Glyma08g03910 |
| CCATTCCCTACAGTTACGAGC (SEQ ID = 73) | AGCTTCACCTGCTGCTTCTG (SEQ ID = 74) | S15851345 | Glyma08g38190 |
| CACGAGAATGGCGTTTTCTTA (SEQ ID = 75) | CCAAAGCCAGAGAAGAGACAA (SEQ ID = 76) | S4943022 | Glyma09g04630 |
| TTGGACGGTTGAATGATTTC (SEQ ID = 77) | CGCCCTAACTTAATCACCCT (SEQ ID = 78) | TC225578 | Glyma09g04630 |
| GGAAGAAGAGCAGGTGTTGG (SEQ ID = 79) | ATCTTGGGCATCCAAGTCAG (SEQ ID = 80) | S22668583 | Glyma09g27180 |
| AGTAATAATATCACCACCGCACC (SEQ ID = 81) | TACTAGTCTCTGGAGAGGCGTT (SEQ ID = 82) | TC234528 | Glyma09g33240 |
| TGTATCTGAGCAATGGAGCG (SEQ ID = 83) | AAGACCAACCGAGTGAAACG (SEQ ID = 84) | BI321654 | Glyma10g33770 |
| TCCAATTTGCCAGAAGAACC (SEQ ID = 85) | CCTCACACCTCTGTAACGCC (SEQ ID = 86) | TC206902 | Glyma10g33810 |
| AACCAAACCAAACCAAACCA (SEQ ID = 87) | GACACAGCCTCCATCCATTT (SEQ ID = 88) | S26574424 | Glyma10g34760 |
| TCTCCTCTGTTTGGCGTTG (SEQ ID = 89) | GCCACTTTCATTCCCTTGTG (SEQ ID = 90) | CF806953 | Glyma10g36760 |
| ATCCAGTCGTACTCGCAAGC (SEQ ID = 91) | ATGCCAATTTTAGAAGAGCGTC (SEQ ID = 92) | S4910467 | Glyma11g01680 |
| AGCTGTGGAAAACCCAACG (SEQ ID = 93) | GAATAATCCTTTAACGCCGTC (SEQ ID = 94) | S22952295 | Glyma11g03900 |
| GGAGAGTGGATCTTGGGTGA (SEQ ID = 95) | CCCATTTATTCCACCCCTTT (SEQ ID = 96) | TC232915 | Glyma11g03910 |
| TCCATGGGAAGTGGTAAGGA (SEQ ID = 97) | GCCCGAATGTATCCAATGTT (SEQ ID = 98) | TC205929 | Glyma11g14040 |
| TTGCAAAGTTAGCAGAGGTTGA (SEQ ID = 99) | TTCCAATATGGAACCACAAGC (SEQ ID = 100) | S5141801 | Glyma11g14040 |
| CGTCGCCAAAGTACTGGTTT (SEQ ID = 101) | TTTTGCCAAGAAATTGTCCC (SEQ ID = 102) | CB063558 | Glyma11g15650 |
| TGCATGAAAGCAAGTGACAA (SEQ ID = 103) | TACCCCTGGAATAACCACCC (SEQ ID = 104) | S15849732 | Glyma11g31400 |
| TTTTTCATCTCCCACTTCCG (SEQ ID = 105) | GTCAAACTAAACGGCGCATC (SEQ ID = 106) | BE609353 | Glyma11g31400 |
| TCCATGTCATCATCCTCTGC (SEQ ID = 107) | CAGCTGCTAGTCAATCCGGT (SEQ ID = 108) | S23062106 | Glyma12g11150 |
| AATGCAGTGTCTGCAACGAG (SEQ ID = 109) | CCTCCCCATTTTCATGCTTA (SEQ ID = 110) | S4861946 | Glyma12g32400 |
| GAAATCCGTCTTCCACGAAA (SEQ ID = 111) | TCTCCTCGTAGCTTGAAGGC (SEQ ID = 112) | TC220118 | Glyma12g33020 |
| CCCAAACCATTTCCTGAGAA (SEQ ID = 113) | CGTGACGTCCCCATAGAAGA (SEQ ID = 114) | S21565746 | Glyma12g33020 |
| CGCTTCCTACTCCTCCCTTT (SEQ ID = 115) | CCATTGTTGGTGCGAGTTTT (SEQ ID = 116) | S6673193 | Glyma12g35550 |
| GCAACAACCAAGTTCCCTTC (SEQ ID = 117) | AGAGAGCGAGTTCTGGGCTT (SEQ ID = 118) | TC215663 | Glyma13g01930 |
| TACAAAACCTGATTTGCCGC (SEQ ID = 119) | TTCCTCGCCTCTAGACCTCA (SEQ ID = 120) | S15927008 | Glyma13g30990 |
| GCACTACTACTACGCATTTTCCG (SEQ ID = 121) | GGTCACAATCCAGACCTCGT (SEQ ID = 122) | S4870460 | Glyma13g34920 |
| GAGATCCGTGGAAGAAGCAG (SEQ ID = 123) | AAATTGGTCTTGGCCTTGG (SEQ ID = 124) | CF807860 | Glyma14g05470 |
| ACAGGTTTTCCACGGATGAG (SEQ ID = 125) | CTTTGCATCAACGCAGACTC (SEQ ID = 126) | S5049738 | Glyma14g06080 |
| AGCTGAAAAGGGGACAACAA (SEQ ID = 127) | AGAAGGCGACGTGCATAAGT (SEQ ID = 128) | S5141710 | Glyma14g06080 |
| AGAGTCGACGCTCTCCAAAC (SEQ ID = 129) | GAAGCTTCTCGAGTTTTGGACT (SEQ ID = 130) | S4867812 | Glyma14g09320 |
| CTCTACCTTGGTCAGCTGGG (SEQ ID = 131) | TGGGATGACCATCAAGCAAT (SEQ ID = 132) | S4898590 | Glyma14g34590 |
| TCGAGATAACGGAAACCGTC (SEQ ID = 133) | TCGTACTCGGACCTAGTGGC (SEQ ID = 134) | BE821939 | Glyma14g38610 |
| CGTTGGATATCGTATGGCG (SEQ ID = 135) | AAAACCAAGAAACACAGCGG (SEQ ID = 136) | S4871445 | Glyma15g16260 |
| CATTCGAGCAACTCGTTTGA (SEQ ID = 137) | AAGGAGCAGCAGAAAGCAAG (SEQ ID = 138) | S16535713 | Glyma16g01500 |
| GAGCCATAGGGAAACGATCA (SEQ ID = 139) | TTGCAGGGAGGAGTTTGAGT (SEQ ID = 140) | BI971027 | Glyma16g04410 |
| CGCAGCTTCTTTGGAGTAGG (SEQ ID = 141) | GCCTCATTGTGATGATGGTG (SEQ ID = 142) | BF598552 | Glyma16g05190 |
| ACGTCAGCATTGGAGCTTCT (SEQ ID = 143) | AATGTGCACTGTGGCAACTC (SEQ ID = 144) | S4984668 | Glyma17g07860 |
| TTGACTCCCCACGTGGCTCT (SEQ ID = 145) | GTCGTCGCCGGAAAGTATG (SEQ ID = 146) | CD392418 | Glyma17g15480 |
| TGGGACAGGGATTAGGAGTG (SEQ ID = 147) | CCCCTTTTCCCCAATAAAAA (SEQ ID = 148) | CA803122 | Glyma17g18580 |
| GACATCTGGGTTGGTTGCTT (SEQ ID = 149) | ACACCCTTCTTCGGATTCCT (SEQ ID = 150) | BE191084 | Glyma17g18640 |
| CCATACGAAGAACCCAGGAA (SEQ ID = 151) | CATTTTAATCCCACCAACGG (SEQ ID = 152) | S21537044 | Glyma18g29400 |
| CTTCCTGAGGATGAAAAGCG (SEQ ID = 153) | CCGGGACTAAGCCTTCTCTT (SEQ ID = 154) | BF426105 | Glyma18g33460 |
| AAAGAGGAGGAAGAGCCTGG (SEQ ID = 155) | AGCCACTTCAACATTCCACC (SEQ ID = 156) | S5146194 | Glyma18g48730 |
| TGGGAACTACCAATCGGAAC (SEQ ID = 157) | AGGTTGATCTTTGACCACGG (SEQ ID = 158) | TC222644 | Glyma18g51680 |
| GCTGGCCTTTCTCATACAGC (SEQ ID = 159) | CCAACCATTCATTCCTCTGG (SEQ ID = 160) | BF423665 | Glyma19g31960 |
| ACGATGTGACAGAAATCAGAGA (SEQ ID = 161) | AGGAGCTTATGGCGTACGAG (SEQ ID = 162) | S5119153 | Glyma19g40070 |
| ATTCCGGAAAACGTCGTTAG (SEQ ID = 163) | AGAGAACCGATGGCACAGAC (SEQ ID = 164) | S5035194 | Glyma19g40070 |
| TCCTTCCATGTCTAGCGGAG (SEQ ID = 165) | TGAACCCAGAAGGAAAATGA (SEQ ID = 166) | TC225489 | Glyma19g45200 |
| AGGCCTATGATTGTGCTGCT (SEQ ID = 167) | TCTCCTTTTCCTGCCACAAC (SEQ ID = 168) | S4912458 | Glyma20g16920 |
| TTCGTAACATGCTTTTCGCA (SEQ ID = 169) | GGTTGCTTTGCCTTTTAGTTTG (SEQ ID = 170) | S15924601 | Glyma20g16920 |
| GACGGAGCGTGAAGAAGAAC (SEQ ID = 171) | AATTCCACGTCAGCACTTCC (SEQ ID = 172) | AI988637 | Glyma20g29410 |
| TTTTCTTCCAGCCAGCAAAT (SEQ ID = 173) | CTGACCCACTACCACCGTCT (SEQ ID = 174) | S4908467 | Glyma20g30840 |
| TCATCCATAAGGGTTGGAGC (SEQ ID = 175) | GTCCATGTCTAAGGAGGGCA (SEQ ID = 176) | TC211971 | Glyma20g33890 |
| GGAAGCTGCTTTGGTCTACG (SEQ ID = 177) | GTTCAACAGAGGCGTGATGA (SEQ ID = 178) | BE556009 | Glyma20g35820 |
| ACCACTCCCTGATCAGATGC (SEQ ID = 179) | TACCCAGCCCATAGTGGTTC (SEQ ID = 180) | S23061605 | Glyma09g11720 |
| CCTGTCTCAGCACCTCCTTC (SEQ ID = 181) | TCTTGATAAGTGTGCCGCTG (SEQ ID = 182) | TC207359 | Glyma02g40650 |
| CGTAGGGAGCAGAAGACCAG (SEQ ID = 183) | AAAAGATACCGCAATGGTGC (SEQ ID = 184) | S21568762 | Glyma02g40650 |
| CATGGGACTGGGAGAGTGTC (SEQ ID = 185) | TCTACTCCTGTCAACTCCTGTGA (SEQ ID = 186) | S4935262 | Glyma02g45100 |
| TTCCCTCTAATGAAGGCGTG (SEQ ID = 187) | CGCGAGGAACATAAACGAAT (SEQ ID = 188) | BU763867 | Glyma03g36710 |
| AGGCAAAGGGTTTTGGAGAT (SEQ ID = 189) | CTAGCGGCTGTTAGCCTGTT (SEQ ID = 190) | S5043967 | Glyma03g41920 |
| CGGATACTCTTTCGTGCCAT (SEQ ID = 191) | TTGAAGACGAAATCGAGGCT (SEQ ID = 192) | S23070360 | Glyma04g37760 |
| AACCAACAATGGCACAGTCA (SEQ ID = 193) | GGATCTAAACCAACTCCGCA (SEQ ID = 194) | S23069218 | Glyma04g43350 |
| GCAAAGTGGTTGGAGTGGTT (SEQ ID = 195) | TCGAAGTTCCCCATTCTCAC (SEQ ID = 196) | BF598372 | Glyma05g38540 |
| GTGCCATCTAGCCTGCACTT (SEQ ID = 197) | TCCATGAGCATGGGTCTACA (SEQ ID = 198) | S4862027 | Glyma05g38540 |
| ATCCGTGCCACCAGATTTAG (SEQ ID = 199) | GTCTCTTCTAATGGCTGCCG (SEQ ID = 200) | S5127363 | Glyma06g39690 |
| AGTATTGCCACCGTCAGAGC (SEQ ID = 201) | TCCTCAAGAAGTGCAGCAGA (SEQ ID = 202) | S23068348 | Glyma07g15640 |
| ACCAAGACAACCTGGAATGC (SEQ ID = 203) | ATATCATCACCAAGCCAGGG (SEQ ID = 204) | BM891891 | Glyma07g15640 |
| TCAAGATGGGGAAGTTCAGG (SEQ ID = 205) | CTGGATTCAGTGGCATTCCT (SEQ ID = 206) | S5133827 | Glyma07g15640 |
| TCTGGTGCCGGAATCTAATC (SEQ ID = 207) | AGTGAACTCTTGGCCTTGGA (SEQ ID = 208) | BG790017 | Glyma07g16170 |
| ACCATCCTCAATTTTGCGTC (SEQ ID = 209) | TCTTGTTTCTTTGGGTTGGC (SEQ ID = 210) | AI440841 | Glyma07g40270 |
| GGGTGGAGAAGTAGGAGCAA (SEQ ID = 211) | TGGGATAACAACTGTGGGGT (SEQ ID = 212) | AI438005; | Glyma08g10550 |
| | | S4866372 | |
| CAGCAACAACCACAACAACC (SEQ ID = 213) | TGAGCTGCTGAACCAAACTG (SEQ ID = 214) | BE440918 | Glyma08g10550 |
| ATGACATGACTCCACGATACG (SEQ ID = 215) | CACCTATGCTGAATCTATCCACG (SEQ ID = 216) | S4981647 | Glyma08g10550 |
| CCAAGATCCGGCTCCTTTAC (SEQ ID = 217) | TGGCTGTACGTGCAAAAAGA (SEQ ID = 218) | S4891658 | Glyma09g08350 |
| GTCTTGCCCATCTTAATCGC (SEQ ID = 219) | TAAGGTTGGGAAATTGTGGC (SEQ ID = 220) | S4939214 | Glyma09g20030 |
| GCCCAACCTTAGTGAGAACG (SEQ ID = 221) | CGAAGGTGTCTTCCCAACAT (SEQ ID = 222) | S6670416 | Glyma10g06080 |
| GGGTAGGGTAGTAACCAAACAGC (SEQ ID = 223) | AAAGGTTTTCAGGGTTGTCTGA (SEQ ID = 224) | BE823048 | Glyma11g15910 |
| AATTTCCCATGGTCAGCAAG (SEQ ID = 225) | GTTGCTTCCGACTAACGTCC (SEQ ID = 226) | S23068849 | Glyma12g29720 |
| ATGCTTTTCAAGCAGTTGGC (SEQ ID = 227) | AACCAAACAGGCTTGGACC (SEQ ID = 228) | S4862156 | Glyma13g17270 |
| CGCCTTATTCAACGCAATTT (SEQ ID = 229) | TTTGCTTCAGCAGTGTTTGG (SEQ ID = 230) | BG238597 | Glyma13g20370 |
| GAATGAGGTTCAGGATGCGT (SEQ ID = 231) | CATTTTGATCCGAGCCATCT (SEQ ID = 232) | TC211634 | Glyma13g30750 |
| GGGTTCCAAGAGATGGGAAT (SEQ ID = 233) | GCGGCATAACACTTCTCTCC (SEQ ID = 234) | S4877094 | Glyma13g35740 |
| AGCAATGGCTTCTTCTGCAT (SEQ ID = 235) | CTCAGAAGCATGAGCACTGG (SEQ ID = 236) | AW761516 | Glyma14g03650 |
| GGGATCGGTGCACTACTAGG (SEQ ID = 237) | TACAAGAATGCTGGGCCAAT (SEQ ID = 238) | S4871774 | Glyma14g03650 |
| CCAGCTGACCTATATGGCTGT (SEQ ID = 239) | TGCTTTTCTTGTGGCTGCTA (SEQ ID = 240) | S22951343 | Glyma15g19980 |
| CGAAGAGAGTGCTGGTTGTG (SEQ ID = 241) | CAGCACTAAAGACTGTTGCGA (SEQ ID = 242) | S4897074 | Glyma17g05220 |
| CGCTCGCAACAGTATCAAAA (SEQ ID = 243) | GCGCCATTGGTAGTAGGAAA (SEQ ID = 244) | S4989599 | Glyma02g44260 |
| TGTCCCTCACTTACCCCATC (SEQ ID = 245) | TGAAACTGCAGGGAGCTTTT (SEQ ID = 246) | S21565486 | Glyma06923920 |
| GTTGTATCCACAACCGTCCC (SEQ ID = 247) | GGTGAGGTTAATGTTCCCCA (SEQ ID = 248) | S23062053 | Glyma13g26240 |
| GGAACCAGAGACGTCGGATA (SEQ ID = 249) | ATGGTCTCACAGCAGCATTG (SEQ ID = 250) | S4876974 | Glyma16g34300 |
| TTTTGAACGAGTCCTCCACC (SEQ ID = 251) | AATTTTCCCATCAAACGCCT (SEQ ID = 252) | S23063969 | Glyma06g01640 |
| CATGCAGAATAGTGGTCGCT (SEQ ID = 253) | ACATGATTTCCGGGTCAACT (SEQ ID = 254) | S4976159 | Glyma11g09370 |
| CGCCATGCTACCAAAACTAA (SEQ ID = 255) | TGCCAGCTAAATTACCCTCA (SEQ ID = 256) | S4938841 | Glyma16g21840 |
| TCTCTGTTGTTTCGCAGGG (SEQ ID = 257) | GAAGTGAACTCCTTCGTGCC (SEQ ID = 258) | S4876683 | Glyma13g19380 |
| ACGCCAACACCAACCATAAT (SEQ ID = 259) | CTTCTTCTTCGACGATTCCG (SEQ ID = 260) | BE473509 | Glyma01g40690 |
| ATGGAGAGGATATCGAAGCG (SEQ ID = 261) | AACGTCACTCTCCGTCAACC (SEQ ID = 262) | S21566169 | Glyma02g37680 |
| TTGTCGATGACACCGTAGGA (SEQ ID = 263) | CAGCCAAGGAATCAGATGCT (SEQ ID = 264) | AI966815 | Glyma09g40520 |
| AGAAAACTGGCCACCACAAC (SEQ ID = 265) | CTTTGGCTGTTCCAGATGGT (SEQ ID = 266) | S23063344 | Glyma10g32150 |
| TCGAGAATGGTTTCCAGAGG (SEQ ID = 267) | AAAGCATCACGGAATTTTGC (SEQ ID = 268) | S5139707 | Glyma13g34680 |
| GAACCAGAAGAAGCAGTGGC (SEQ ID = 269) | TCAGACAGCTTGGGTGTGAG (SEQ ID = 270) | S5115432 | Glyma18g07510 |
| GGCTTCTAAGGCACAGGTTG (SEQ ID = 271) | TGGTTTCCCATCCACTTCAT (SEQ ID = 272) | S5146625 | Glyma01g02350 |
| GTCACCCAAGTAACCCACCA (SEQ ID = 273) | AGGGCATTTTCTCATGCCTA (SEQ ID = 274) | S22951976 | Glyma01g24100 |
| CGCCATGACAACATAAAACG (SEQ ID = 275) | GAAGCGAGAACTGAAGGCAT (SEQ ID = 276) | S23061455 | Glyma04g09550 |
| CCCGAGTTAATGTTATGGTTGA (SEQ ID = 277) | CTGTGAATGCTGCGACTACG (SEQ ID = 278) | S35599000 | Glyma04g09550 |
| AGAGAACCAGTCGGTGATGG (SEQ ID = 279) | TAGGCGTCAAGGCCATTTTA (SEQ ID = 280) | S5101674 | Glyma06g17320 |
| GGCATTCTCGGAAATTGATG (SEQ ID = 281) | CACCCCACCACTTGACTCTT (SEQ ID = 282) | S5146871 | Glyma08g22190 |
| AAGCTTCCTTGGGAGAGAGG (SEQ ID = 283) | GCTGCGGAATTAGGAGTGAG (SEQ ID = 284) | S23064650 | Glyma10g03720 |
| GCAGCATCACCTTCCTCTTC (SEQ ID = 285) | ATTGGCAACAAGAGAATCGG (SEQ ID = 286) | BM732148 | Glyma10g04610 |
| GATACCCATAATTCGCACGC (SEQ ID = 287) | TCATCTCCTCGTGCTTGTTTT (SEQ ID = 288) | CF806335 | Glyma10g30440 |
| TATGCTCAGAGGGCCTGTTT (SEQ ID = 289) | ACGAGCTTTCCTCCCAAATC (SEQ ID = 290) | S15931785 | Glyma11g20490 |
| TGTTCACCTGCTGAAACTCG (SEQ ID = 291) | CGCACCTAGCTTCATTCCAT (SEQ ID = 292) | S4875111 | Glyma13g43050 |
| CGTCACACGTGTACCTGCTT (SEQ ID = 293) | GGTGAACGGTTTAGCGTGTT (SEQ ID = 294) | S5080036 | Glyma14g09390 |
| CCTTGCAAAGCTCCACTGTT (SEQ ID = 295) | CTGTGTCCGCTGCATAAGAA (SEQ ID = 296) | BE823122 | Glyma17g37580 |
| GTTAAGGCTTGGACTGCCTG (SEQ ID = 297) | GCATCAAATCCACAGTGGTG (SEQ ID = 298) | S5146870 | Glyma19g34380 |
| GTGAGCACCCAAATCAACCT (SEQ ID = 299) | GGAAACCTCAGGACTTCCCT (SEQ ID = 300) | S5139519 | Glyma19g35180 |
| TTTTCTGATCAGCGACCTCA (SEQ ID = 301) | TGACACTGCCTCTTCCTTCA (SEQ ID = 302) | S5129544 | Glyma19g40970 |
| TGGGTGCTAAGCTGTGTGAG (SEQ ID = 303) | CAAAGCTCGGTCTCCTTGAG (SEQ ID = 304) | S4878791 | Glyma20g35270 |
| CTATCTTCGTCCATGACCCC (SEQ ID = 305) | AGTTGCATGACCTCCCAAAG (SEQ ID = 306) | S23068785 | Glyma02g18250 |
| TCCCAAAACTCCACACATGA (SEQ ID = 307) | TGGTGAGGGTTTGAAGAAGG (SEQ ID = 308) | S5142874 | Glyma19g38340 |
| GGCCAAGAAGAACCCATGT (SEQ ID = 309) | GGGGTCCACCGAGTTAATTT (SEQ ID = 310) | S5126647 | Glyma01g02250 |
| ATGGGAAGACAAAGTCACCG (SEQ ID = 311) | GACTTCAAATTCGAGGCCG (SEQ ID = 312) | BF325042 | Glyma01g02250 |
| CTTTGTTTCCTCGTTTCCCA (SEQ ID = 313) | AGCGCTACAAAGTGCTGGTT (SEQ ID = 314) | AW310700 | Glyma01g09010 |
| CTGAGTGATGCCATGGAGAC (SEQ ID = 315) | CTGAACCCAACCATTCGTTT (SEQ ID = 316) | S4891278 | Glyma01g09010 |
| ACCGTAGACGACCACGATTC (SEQ ID = 317) | GTGGACACCGATGATTTTCC (SEQ ID = 318) | S5028099 | Glyma01g15930 |
| TGCATCAATTATCACGCACA (SEQ ID = 319) | TGGTGCAATACGTAGCCTTT (SEQ ID = 320) | S4930680 | Glyma02g37310 |
| ACGACCGTGATTCCATTAGC (SEQ ID = 321) | TGATTCTTTTGTTGGACCCAG (SEQ ID = 322) | S18957200 | Glyma03g04000 |
| TGTACTTAAGCTACTGGCCAAGC (SEQ ID = 323) | GGTGTGCACCTACCATAGCA (SEQ ID = 324) | TC229276; | Glyma03g25280 |
| | | S7107502 | |
| ATTCGTTAGCGTGGCTCATT (SEQ ID = 325) | GATGGACCATGAATTCAGCA (SEQ ID = 326) | AW309251 | Glyma03g25280 |
| GAAAGGTCCTCTGCACCATC (SEQ ID = 327) | GTCATTAACCTTCTTGCGGC (SEQ ID = 328) | BQ611037 | Glyma03g28630 |
| TGATTGGCTCTTTACGAGGA (SEQ ID = 329) | TGCTTTGTGATTTGAATGGG (SEQ ID = 330) | BE473577 | Glyma03g29710 |
| TGACGTCATCGTCAAATCGT (SEQ ID = 331) | TTCGGAGACAGTAAGGAGCG (SEQ ID = 332) | S5014134 | Glyma03g32740 |
| AAAGTATCATCCGGTGCAGG (SEQ ID = 333) | TAATTAAGGTGGGAAGGGGG (SEQ ID = 334) | CA785248 | Glyma03g41900 |
| AGTTGGAGGAAAGGAGAGCC (SEQ ID = 335) | ACTCATGAAGCCCATCCAAG (SEQ ID = 336) | S4885609 | Glyma05g37770 |
| GCTTACCTCCTCAACATGGG (SEQ ID = 337) | AGGGAAAAGATGTAGCCGGT (SEQ ID = 338) | S5015816 | Glyma06g01430 |
| TAGCATCAAGATTCGGTTCG (SEQ ID = 339) | TCACATGAATTTTACCCCCTG (SEQ ID = 340) | S21565817 | Glyma06g17330 |
| CCCTCAAGGAAGCATTACCA (SEQ ID = 341) | CCTGTGCCATCTTCACCTTT (SEQ ID = 342) | BM732581 | Glyma06g44660 |
| ACGATGAAGACACCACCTCC (SEQ ID = 343) | CTCAATGAGCACCTCCTTCC (SEQ ID = 344) | S4904362 | Glyma07g03060 |
| GCAGATTGACTGCTCATGATGT (SEQ ID = 345) | GGGGCTTTCGTTAGGAGTTT (SEQ ID = 346) | BI970205 | Glyma07g09180 |
| CCTCGCATCGGAGTTATTGT (SEQ ID = 347) | GAGTTTCAACCAGCAAAGCC (SEQ ID = 348) | S23071477 | Glyma08g04110 |
| CTACTGCCAAAGGCCTGAAG (SEQ ID = 349) | TTCATTGAGTCGATCCCTCC (SEQ ID = 350) | BU965443 | Glyma08g15740 |
| AATGGTGGATCTTCCAGTGC (SEQ ID = 351) | TGGAGCAATTCCTGATACCC (SEQ ID = 352) | TC217902 | Glyma08g16190 |
| AAGATTCCGTTCCTTGCAGA (SEQ ID = 353) | CACTGATACGAGTCCTGCGA (SEQ ID = 354) | S5093793 | Glyma08g26110 |
| GAACGTGCTATTGCTGGGTT (SEQ ID = 355) | AATTGATGTGGGGAGACGAG (SEQ ID = 356) | S5142763 | Glyma08g28010 |
| TGAAGGATGGAATCAGGAGC (SEQ ID = 357) | CACTGAAGTTGCCACAATGC (SEQ ID = 358) | AW507968 | Glyma08g28010 |
| GCCGAGAGACAGAGGAGAGA (SEQ ID = 359) | ATGTACAATATGGCGTCCCC (SEQ ID = 360) | S4865763 | Glyma08g36720 |
| CACCCAGAAAACATCAATGG (SEQ ID = 361) | CAGTGACAGCTCCATGCCTA (SEQ ID = 362) | S4877270 | Glyma08g40540 |
| TGCTGTTGCTGGGTGTAATC (SEQ ID = 363) | AAAATGCCTCTCAGCCAATG (SEQ ID = 364) | CD398155 | Glyma08g41620 |
| ACCCTCTTGGCAATCATCAC (SEQ ID = 365) | CATGTGGGGGTGTTGTTGTA (SEQ ID = 366) | S5025226 | Glyma08g46040 |
| GATGAACAAGGGAAGGGCTC (SEQ ID = 367) | ACTTGGGATCGTTAACCAAA (SEQ ID = 368) | TC223273 | Glyma09g33730 |
| GGATCTAAAGCTTGCCGTGA (SEQ ID = 369) | GTTCTCACAGGTCTCCCTGG (SEQ ID = 370) | CF805700 | Glyma10g01010 |
| AACCAACAAAGAACAGGTTAGC (SEQ ID = 371) | TGCACTAATGACTCAGTTGAAGG (SEQ ID = 372) | S23069022 | Glyma10g01780 |
| TTTTGGGAATTTTGGCTCAG (SEQ ID = 373) | TCACCCACCATCTTTCTTCC (SEQ ID = 374) | S5143908 | Glyma10g03950 |
| CGAGTTCCTCTTCCCACATC (SEQ ID = 375) | TGCAACGAAGTTTTCTCCCT (SEQ ID = 376) | S21566702 | Glyma10g04890 |
| TAGGGGGCAGAACATGAATC (SEQ ID = 377) | GTTGGCAGGTGCAGTTCTTT (SEQ ID = 378) | BU550119 | Glyma10g04890 |
| ATCCAGGGCCATATTGTTGA (SEQ ID = 379) | CTTCTTCGCTCGGAATGTGT (SEQ ID = 380) | S23062909 | Glyma10g12150 |
| ACCAAGGTTCAGAAGAGCCA (SEQ ID = 381) | GCACCAGCTGATTCTTCCTC (SEQ ID = 382) | S4974129 | Glyma10g28290 |
| CCCATCATTGCATCAGTGTC (SEQ ID = 383) | CCATAAGACGCATCCTGGTT (SEQ ID = 384) | AW760679 | Glyma10g28290 |
| GGGCTCCTCCGATTTTACTT (SEQ ID = 385) | ATCTAGTCGGTGCAGCTGGT (SEQ ID = 386) | S21538929 | Glyma10g30430 |
| CATCCTTGTCCAGGAGGTGT (SEQ ID = 387) | CCACATCAAGCCCTTCCTTA (SEQ ID = 388) | BE020687 | Glyma10g38620 |
| AATTCACTGCCTCGCTCATT (SEQ ID = 389) | AAAGGCAAAGGAGGCAAGA (SEQ ID = 390) | BI968952 | Glyma10g38630 |
| TGAATGTGAAACCAAACCCA (SEQ ID = 391) | GGTGAGGTGGAAAATGGAAA (SEQ ID = 392) | S23065851 | Glyma11g13960 |
| ACAGCATGGGAATAAGCCCT (SEQ ID = 393) | CAAGAAAAGTTTCGGGCAAA (SEQ ID = 394) | S5011517 | Glyma12g04670 |
| CTACTCGTATGCCACGCTCA (SEQ ID = 395) | GCCATTGGTGTTGATGGTAA (SEQ ID = 396) | S4898095 | Glyma12g09990 |
| TGATCGACGATATTCCCGTT (SEQ ID = 397) | AACACCGACATTGGAAGGAG (SEQ ID = 398) | S4897794 | Glyma12g16560 |
| GATACCAGTAACCGGAAGGC (SEQ ID = 399) | ATGTCAGTCATTCAAGCGCA (SEQ ID = 400) | S4861813 | Glyma12g31460 |
| TGTCGTGAGAAATTGCGAAG (SEQ ID = 401) | AGCCGCATCGCTTAATAATG (SEQ ID = 402) | S6671401 | Glyma12g32280 |
| TTAATTCCTCGCACGAGCTT (SEQ ID = 403) | TCGTTTGGGAAAAACAGGTC (SEQ ID = 404) | S4874826 | Glyma13g00480 |
| CCAATGGGACTTTAGGTGTCA (SEQ ID = 405) | ATCTAGACAAGGAACCCCGC (SEQ ID = 406) | S5093492 | Glyma13g18130 |
| AACAGGCAAAACGACGAGAT (SEQ ID = 407) | TTCTGAAGGGTCGTTGGTTC (SEQ ID = 408) | AW734878 | Glyma13g19250 |
| AAAACCTCTCTTGGCACGAA (SEQ ID = 409) | TTTGAGTCTGCCTGGCTCTT (SEQ ID = 410) | S5129107 | Glyma13g27460 |
| CAATGCCAAGCTATGCACAC (SEQ ID = 411) | TCCCAGCACTCTTCTTTGCT (SEQ ID = 412) | TC209223 | Glyma13g27460 |
| ATTAGCCACTGGGAATGTGC (SEQ ID = 413) | GACTCAGAAGGGGCAAAACA (SEQ ID = 414) | BU547516 | Glyma13g32320 |
| CTCCCGGATAGCTGATGAAA (SEQ ID = 415) | TCAATGAATGCTCAACCTGC (SEQ ID = 416) | S23061550 | Glyma13g36260 |
| GATTCGCTCCATCATCACAA (SEQ ID = 417) | GTGTTCCTCGTTGACGCTCT (SEQ ID = 418) | TC216048 | Glyma13g41670 |
| CCACTATAGGATTCCATGACTGA (SEQ ID = 419) | AATCGACAGCGTACTTCAACTG (SEQ ID = 420) | BU546499 | Glyma14g06830 |
| GTGCAATTGCCTCATCTTCA (SEQ ID = 421) | TTCACGGAGGGTACACCAAT (SEQ ID = 422) | BG352463 | Glyma14g09230 |
| AACGGGACAGACTCATGCTC (SEQ ID = 423) | TGCACGACCAGAATCTGAAA (SEQ ID = 424) | S5055402 | Glyma15g03740 |
| GGAACAACCAAGCAAGCTCT (SEQ ID = 425) | AGTCCAGGAACACGGTCATC (SEQ ID = 426) | S5025536 | Glyma15g18580 |
| CACGTGACCGTGAGCTTTTA (SEQ ID = 427) | TGCCCACTTTCTCAGATTCC (SEQ ID = 428) | S21700422 | Glyma15g33020 |
| GACTCCTCCCCCTCTTTCAG (SEQ ID = 429) | CTGGCCTCCACTTCATGTTT (SEQ ID = 430) | TC217569 | Glyma16g05390 |
| GCTAATTCCTCCCAATGCAG (SEQ ID = 431) | TGCTATCCCAATAGACGCAC (SEQ ID = 432) | S22951832 | Glyma16g26290 |
| ACGTGTTCTGCGAGGACTTT (SEQ ID = 433) | GGCTTCCACCAGAAACAAAA (SEQ ID = 434) | S23066270 | Glyma17g07640 |
| TCAGCAACTACCCCCAAGAC (SEQ ID = 435) | CCACCTGGACCACCTATTTG (SEQ ID = 436) | BM885371 | Glyma17g08980 |
| TCAGCATCAATGCTCTCGTC (SEQ ID = 437) | AGCAAGAAAACAAGGGCAGA (SEQ ID = 438) | S23070422 | Glyma17g16720 |
| GGGGTACGGCATAGTCAAAC (SEQ ID = 439) | ATTTTGCCACTCACAGCCTC (SEQ ID = 440) | S4937428 | Glyma18g14530 |
| ATGAAAATGCCCTACCTGCC (SEQ ID = 441) | TCATTCTAGGTGTGCTGAGAGC (SEQ ID = 442) | S15849327 | Glyma18g49320 |
| GGTGGGTGTTTAAGGCTGAC (SEQ ID = 443) | ACGCGCATATATGATCACCA (SEQ ID = 444) | S4932282 | Glyma19g27480 |
| GTGTTCTTTGTCAGCAGCGA (SEQ ID = 445) | CTCATCCCCGACCTCATAGA (SEQ ID = 446) | S4936213 | Glyma19g30910 |
| TTCCCCACACACATTCTTCA (SEQ ID = 447) | TGAACCGTACACACCTCGAA (SEQ ID = 448) | BG362671 | Glyma19g32570 |
| TTAAAAGCTGGCATTCTGCAT (SEQ ID = 449) | CCAAACATGAATAGGACCCG (SEQ ID = 450) | S21565183 | Glyma19g32600 |
| TTGTGTGGCAGAATTTCCAA (SEQ ID = 451) | TTGGTTCCCCAAACCAAATA (SEQ ID = 452) | S4994398 | Glyma19g40980 |
| TGGAGGAGCTTGGAGGAGTA (SEQ ID = 453) | TTCCGTTAACAATAAGCGCC (SEQ ID = 454) | S23064706 | Glyma19g41580 |
| GCTCCAAAACCAACACCAAT (SEQ ID = 455) | GCAATAGCTTGTCCACGGTT (SEQ ID = 456) | S4911216 | Glyma20g39220 |
| CCGTCGTCTTCCTCTACTGG (SEQ ID = 457) | GGGGGAAATGTTGGAGAAAT (SEQ ID = 458) | TC205627 | Glyma02g01600 |
| TAGAGGCTTTGGAGCAGGAA (SEQ ID = 459) | ACCAATAGCACCCAAACGAG (SEQ ID = 460) | S34818003 | Glyma02g09140 |
| AGGCTCCGACAAAGACAAGA (SEQ ID = 461) | CTCTCCCTTGACCTCACAGC (SEQ ID = 462) | S34818022 | Glyma02g19870 |
| TCCAACATGAAGGCTGAAGA (SEQ ID = 463) | TAGTACACGGGCACAAATCG (SEQ ID = 464) | S5104924 | Glyma02g39780 |
| TTTAGAAGCTGGGCTTGACC (SEQ ID = 465) | AACAACGCATGACAAGGGAT (SEQ ID = 466) | TC206111 | Glyma03g27860 |
| TCTGGCATGTGCACTGAGTT (SEQ ID = 467) | GTTTCGGTGAAACATTGGCT (SEQ ID = 468) | S4865864 | Glyma03g27860 |
| GCTATTGCTGGGTCTCAAGC (SEQ ID = 469) | CTCTCCCCAGTTCTCACGAC (SEQ ID = 470) | S34818015 | Glyma03g28320 |
| TATGACTCGGGGATCTTTGG (SEQ ID = 471) | GGTAGCATGCGATCCAACTT (SEQ ID = 472) | S34818013 | Glyma03g40730 |
| GATTTCTGGCTCACATCCGT (SEQ ID = 473) | CAGCGCTCAAGAAGGAGAAG (SEQ ID = 474) | S4864503 | Glyma03g40730 |
| TGGGTACAGAATGAGCGTGA (SEQ ID = 475) | TTGTCGTGCCAGTTCTTCAG (SEQ ID = 476) | S4881352 | Glyma03g41590 |
| TGGGTACAGAATGAGCGTGA (SEQ ID = 477) | TCAGTTTCAGCCTGCTTCCT (SEQ ID = 478) | S34818019 | Glyma03g41590 |
| TTCTAGCTCTGGACCGAACC (SEQ ID = 479) | CCTCCGGCTCTAAGAAAACC (SEQ ID = 480) | S15937626 | Glyma04g02420 |
| AACCAACCCGTTTTTCAGTG (SEQ ID = 481) | GAGAAGATTCACCCAGACGC (SEQ ID = 482) | TC209970 | Glyma04g03200 |
| TCTTGCCACCCATTGGTTA (SEQ ID = 483) | TTGGACACAATCTCACCGAA (SEQ ID = 484) | TC229348 | Glyma04g04170 |
| TCAAGTGGCCAAATAGTCCC (SEQ ID = 485) | TCAGCACTTGGAAACTTGGA (SEQ ID = 486) | S23070844 | Glyma04g08290 |
| GCTAATGGTAAGGCCCATGA (SEQ ID = 487) | TTCAACACCCCAAAAGGAAG (SEQ ID = 488) | S4866994 | Glyma04g08290 |
| GAACCTGCTACGCCAAAAAG (SEQ ID = 489) | TGTTGTTGTTGGTGCATGTG (SEQ ID = 490) | S5132128 | Glyma05g22860 |
| TCTTCTCCAGTGATCTCCGA (SEQ ID = 491) | ATTGCACCAAGTGTGTCCTG (SEQ ID = 492) | TC216155 | Glyma05g28960 |
| AGGGCTCATCAGGTTTCAGA (SEQ ID = 493) | TGGGAAACACTAGGAAACGG (SEQ ID = 494) | S34818035 | Glyma05g30170 |
| CCAAATCTTGAGCAGGCTTC (SEQ ID = 495) | AGGCCCTCCAACCTGTTAAT (SEQ ID = 496) | S34818007 | Glyma06g01240 |
| GCACAGTTAATGAAGTTACCCG (SEQ ID = 497) | ACCAGGTAAAAAGCCCATCC (SEQ ID = 498) | BU761457 | Glyma07g06620 |
| CTTGGGAATTGTTTCCTCCA (SEQ ID = 499) | AAAGATGGACAGGTTCCGTG (SEQ ID = 500) | S4864656 | Glyma07g33600 |
| CTTCCACAAGCAGTGGATCA (SEQ ID = 501) | CATTGCAGGTTCTCGGAGTT (SEQ ID = 502) | S5140472 | Glyma08g08220 |
| GGTATGGGGTGAGGTACACG (SEQ ID = 503) | TGTATCCACCGAGTCATACAACA (SEQ ID = 504) | S4974571 | Glyma08g08220 |
| TTCACCCAAATCAAGCAGAA (SEQ ID = 505) | TGTGAGCTTTGTGAACCAGG (SEQ ID = 506) | S21567935 | Glyma08g14840 |
| TCAATCAGCTCATGGAGTGC (SEQ ID = 507) | GGGATGAATTCACTCTCCGA (SEQ ID = 508) | BM524950 | Glyma08g19590 |
| TTTCTTCCAGGAGTCTGCGT (SEQ ID = 509) | TACAGCCATTACACATGGGG (SEQ ID = 510) | S4989510 | Glyma08g24340 |
| TGGTGGTGGTGGAGACAGTA (SEQ ID = 511) | CAAATCGCCCAATTGATTCT (SEQ ID = 512) | S4957187 | Glyma08g24340 |
| CCTAACCAAGTAGCAACAGCAA (SEQ ID = 513) | CATGACAAATTAGGAATGAGGG (SEQ ID = 514) | TC218693 | Glyma08g34280 |
| TAGACTGCTTCCGCCTTTGT (SEQ ID = 515) | AGTTGCTGGAGGGATGATTG (SEQ ID = 516) | S23064509 | Glyma08g34280 |
| TATGAGCCAGTCTTGTCCCC (SEQ ID = 517) | AGCATCGGTCATCATATCAATC (SEQ ID = 518) | S5146449 | Glyma08g41450 |
| TGTGCTCTGAGGATCATTCG (SEQ ID = 519) | GATGAAGAAGCCGAAGTTGC (SEQ ID = 520) | S15850391 | Glyma08g45670 |
| TCCAGCTTTGGAAGATCCAC (SEQ ID = 521) | ATCCATCTCACTGCTTCCCA (SEQ ID = 522) | TC220458 | Glyma09g34170 |
| CTCGAGTTGGACCTCGAAAC (SEQ ID = 523) | AGAGACTCTTTGGACCGCC (SEQ ID = 524) | S34818018 | Glyma09g37800 |
| CATAATGGGACGTGAAGTCG (SEQ ID = 525) | GCTTGCGTAGTCTTGATCTCC (SEQ ID = 526) | S5146765 | Glyma11g06960 |
| TGGTAATGTAGAGGGGTCCG (SEQ ID = 527) | TCGGTTCCAGAAGAGTTCAAA (SEQ ID = 528) | S34817997 | Glyma11g11790 |
| TTGCGTTTCAACCTCTTCCT (SEQ ID = 529) | GGGATGGGAGGAGATTTGTT (SEQ ID = 530) | S4891443 | Glyma11g12250 |
| CGTCTTGCACAAAATCGAGA (SEQ ID = 531) | TGCACGTTCAAGTTCTTGCT (SEQ ID = 532) | S34818027 | Glyma11g36010 |
| AGATGCGGTACATTTCGGAG (SEQ ID = 533) | GGTTAGTGAGTCCAGCCGAA (SEQ ID = 534) | TC216103 | Glyma12g04050 |
| CTCGTTTTTCTCGCTCGACT (SEQ ID = 535) | GATCTTCCATGGACACGTCA (SEQ ID = 536) | TC232817 | Glyma12g04050 |
| GTGGGAAAGGAAGGATCACA (SEQ ID = 537) | CTGACAACTGCTCAAGCTGC (SEQ ID = 538) | BE821907 | Glyma13g02360 |
| CTCCGGGTTCTGTTCACATT (SEQ ID = 539) | ATCGCAACCTATGCAGCTCT (SEQ ID = 540) | S34818014 | Glyma13g26280 |
| GATGTTTTGGGTGGGTTTTG (SEQ ID = 541) | AGCATCAACCCAAACTGTCC (SEQ ID = 542) | S16523242 | Glyma13g42030 |
| AGGAAAAGGGGGTTGGTATG (SEQ ID = 543) | AAAACCCACCCAAAACATCA (SEQ ID = 544) | TC208796 | Glyma13g42030 |
| CATGAATGATTCCACCGTGA (SEQ ID = 545) | TCTTAACCAACCAATTGTGGC (SEQ ID = 546) | S5139088 | Glyma14g07800 |
| CATGGAGCAACAAGCACAAC (SEQ ID = 547) | GGAATCAGTGTGGCTCATCA (SEQ ID = 548) | TC221650 | Glyma14g38460 |
| TAGGGTGCTGCTGTTCCTTT (SEQ ID = 549) | ACGGTCAGAACTTGGTGGAG (SEQ ID = 550) | S23063669 | Glyma14g40580 |
| TTCAGGACTCATCCCCAATC (SEQ ID = 551) | GCTGGGTTGCGCTTATTTTA (SEQ ID = 552) | S4993988 | Glyma15g01790 |
| TGCTGGCGAGAAGTAGAAGG (SEQ ID = 553) | ACATGCTCCATCATTGCTGA (SEQ ID = 554) | BQ786172 | Glyma15g27040 |
| GATTGATGGACGCGCTAAAT (SEQ ID = 555) | GTGATGCAGAGAGGACAGCA (SEQ ID = 556) | S4911209 | Glyma15g37220 |
| CTTGTCGGCCGCTGTATAAT (SEQ ID = 557) | CCCAAAGTCAGAATGCCTTG (SEQ ID = 558) | S5146764 | Glyma16g03190 |
| CGAGGCCAAAAACTGATGAT (SEQ ID = 559) | TTTGACGCACCCTCTAGCTT (SEQ ID = 560) | S34818001 | Glyma16g13570 |
| CCTGATTGGTCAAGCTCCAT (SEQ ID = 561) | AAATAGGGATGGGGAGTTGG (SEQ ID = 562) | S5019309 | Glyma16g25600 |
| GCCACTGCAGACAACAACAT (SEQ ID = 563) | ATTCCACCGTGACGAAACTC (SEQ ID = 564) | S4890532 | Glyma17g37180 |
| CTTGTCCCCAGTGCAAGACT (SEQ ID = 565) | TCAGCATCGTCTTCGTCATC (SEQ ID = 566) | S34818031; | Glyma18g14750 |
| | | S5146448 | |
| CACCTGAGCCTAAGCCAAAG (SEQ ID = 567) | GCATGGGCAAGAATTAGGAA (SEQ ID = 568) | S5076266 | Glyma19g20090 |
| TTGAGGACTCTTGCAGCTTG (SEQ ID = 569) | AGTCAAAGCCGGTTGAAGAA (SEQ ID = 570) | BU545299 | Glyma19g37910 |
| TCAGATCCTCTCCTCAAGCC (SEQ ID = 571) | CCCAAACGAAGAAAGAGCAA (SEQ ID = 572) | S4865594 | Glyma19g40390 |
| CGCCATGACTAGGGGATCT (SEQ ID = 573) | GAGAAGGATTAGTCGGCTGTG (SEQ ID = 574) | S34818017 | Glyma20g36750 |
| CCAGCAGCACAACAGGAGTA (SEQ ID = 575) | CCAGCACTGGTTGCATATTG (SEQ ID = 576) | S23066857 | Glyma11g13690 |
| CTCTGTGCCAAAGGATTGGT (SEQ ID = 577) | GGAGGGAGCACATAGGTTGA (SEQ ID = 578) | AI440589 | Glyma07g39930 |
| TCATTATCGGTATTCGGCGT (SEQ ID = 579) | GTCTCGAATTTGTGCGGAAT (SEQ ID = 580) | CF808139 | Glyma02g16840 |
| GTTGATGTCCTGGAGAGGGA (SEQ ID = 581) | TGTGCAAATCATTGGCTGTT (SEQ ID = 582) | BM528163 | Glyma02g45260 |
| ACACATTCGGGTATTTCCCA (SEQ ID = 583) | AGCTTCAATGCATGCCTCTT (SEQ ID = 584) | TC212833 | Glyma02g47680 |
| CAAGATCACTGCCAAGGACA (SEQ ID = 585) | CGCCAAAATGAATTGGGATA (SEQ ID = 586) | S21567300 | Glyma04g42350 |
| CCATGAGTTAACCTATACCGGG (SEQ ID = 587) | TTCCAGCATGCAGATAAGGA (SEQ ID = 588) | S5127388 | Glyma06g12140 |
| ACAGCACATCATGGTACGGA (SEQ ID = 589) | CATCACCAAGTCTGACGCAT (SEQ ID = 590) | BI786004 | Glyma06g12440 |
| TCTTTGCCCAAGCTATGCTC (SEQ ID = 591) | CACAACTCATTCCTGTGCTG (SEQ ID = 592) | TC208469 | Glyma06g45770 |
| TCAAGAAACCAAAACTCCCC (SEQ ID = 593) | CTTCCCTTTTCCTCGACAGA (SEQ ID = 594) | S5055004 | Glyma12g30500 |
| TGCTCTTCTTCACTGCCCTT (SEQ ID = 595) | TGAGAATGGTAGGCGCTTCT (SEQ ID = 596) | S4993306 | Glyma14g03510 |
| ATATACGATGTGGCATCGGG (SEQ ID = 597) | CGAGAAGCTACATGCAAAGC (SEQ ID = 598) | S5022954 | Glyma14g05000 |
| ATACTGCATTCCTTGGTCGC (SEQ ID = 599) | GGCCATACAGATCTGGTTTCA (SEQ ID = 600) | S4980150 | Glyma14g23960 |
| GCCTTGTGGACGTCATCTTT (SEQ ID = 601) | GGAGGATGACTTGCCTGACT (SEQ ID = 602) | S4934562 | Glyma15g13320 |
| GAAATAGGGTGCCATGCAGT (SEQ ID = 603) | CTTTTGCTGCCTTCTGTTCC (SEQ ID = 604) | CA802838 | Glyma18g00840 |
| CCATGCAAGAATGTGTGTCC (SEQ ID = 605) | AGCAAATATCGTCGCCATTC (SEQ ID = 606) | S4863935 | Glyma02g17310 |
| AAGGTTGGAGCAGTGACCTG (SEQ ID = 607) | CTTGGATCTTCCGTCCACTC (SEQ ID = 608) | S4925563 | Glyma02g35190 |
| ATGGAGGGAGAGAAGACCGT (SEQ ID = 609) | GCACTTGATGATGGTAGGCA (SEQ ID = 610) | S4912143 | Glyma02g46970 |
| CCGAGAGATGGAGGGTGATA (SEQ ID = 611) | GCTGAGCATTAGGACTTGGC (SEQ ID = 612) | S4904793 | Glyma03g33490 |
| ACTGGCGTGGAAAACATACG (SEQ ID = 613) | GGGTACCTGATCCTTAAATTGG (SEQ ID = 614) | S15847588 | Glyma03g33490 |
| GAAACATGTATGAGCATCTGCC (SEQ ID = 615) | CCCTCCCTCTACCTCACCTT (SEQ ID = 616) | S4900633 | Glyma06g17780 |
| GCAGCATCTCTTACTCTTCCC (SEQ ID = 617) | AATGGGCGAGTACATTCACG (SEQ ID = 618) | S4891274 | Glyma06g23240 |
| AGTGGAGCTACCAGCCTGTC (SEQ ID = 619) | ACCATAACCAACTTGGGTGG (SEQ ID = 620) | BU760757 | Glyma06g23240 |
| AACTGCACAACTGAAGCCCT (SEQ ID = 621) | TGCAGTGATGAGTTTTTGGG (SEQ ID = 622) | CD411387 | Glyma07g37830 |
| CTGTAGCTGTTCCTTCCCCA (SEQ ID = 623) | CTGCTGTTGTTGGTGTTGCT (SEQ ID = 624) | S4996612 | Glyma08g17630 |
| TGCAGGCTACTTTCCAACCT (SEQ ID = 625) | CATACACAACCCCTGCAACA (SEQ ID = 626) | CK605647 | Glyma08g17630 |
| CACTCTTCAATTTCAAACGCAC (SEQ ID = 627) | ACTGAGAAAGCGAGGTTTGC (SEQ ID = 628) | BE659926 | Glyma08g17630 |
| CTAGGTTCAAAGGCCAACCA (SEQ ID = 629) | AGGGAAACTTGACACCATTTG (SEQ ID = 630) | TC209551 | Glyma08g44140 |
| ACCAGAATGTGCACCAGTGA (SEQ ID = 631) | TGCTTTGAATAGGGTTAGGGG (SEQ ID = 632) | S4994511 | Glyma09g07960 |
| CTGGATTTCTGACTTTGTGTGG (SEQ ID = 633) | TGGAGGGTAAGTCCAGATCG (SEQ ID = 634) | S5108906 | Glyma10g10240 |
| CCATGGCCCATAGTAAATCG (SEQ ID = 635) | AGACACAATGCAAGAATGCG (SEQ ID = 636) | S23064915 | Glyma10g33550 |
| TGAGCCGAGAAAGAAAAGGA (SEQ ID = 637) | TCACCTTAATCACTCTCACCGTT (SEQ ID = 638) | S4909265 | Glyma11g18960 |
| CCAAGGCTTGTGACCTCTTC (SEQ ID = 639) | GTGCAAAGTCCTCCTTTTGC (SEQ ID = 640) | AW831868 | Glyma12g34510 |
| GCTGAACTGTGGCTTGTGAA (SEQ ID = 641) | GGCAACAATACTCGTGCAAA (SEQ ID = 642) | S4935933 | Glyma12g36540 |
| TTTAGAAACACACCCGCTCC (SEQ ID = 643) | TGTCACATCACCATCCACAA (SEQ ID = 644) | TC211034 | Glyma15g12570 |
| TAAGCCAAGGATGATTTGCC (SEQ ID = 645) | ACTCACCTTTGGTGGTGGAG (SEQ ID = 646) | S5141662 | Glyma13g16770 |
| CCCTAGCTGGTTTTGTTAGCTT (SEQ ID = 647) | CAAATAGCTGCAGCAAAGCA (SEQ ID = 648) | CA800598 | Glyma04g06620 |
| GAACGCATCCCTCAACTTTC (SEQ ID = 649) | GTTGAACAAGCTTGCGGAGT (SEQ ID = 650) | S6672372 | Glyma06g06700 |
| GCTGATTCGTCAAGTCATCG (SEQ ID = 651) | GGTAGGGTTTTGTGGGGTCT (SEQ ID = 652) | S6681156 | Glyma12g31300 |
| GCTGAAGCCCTGACTTGTTC (SEQ ID = 653) | TTGACACTGACTGGAACCCA (SEQ ID = 654) | S23070450 | Glyma07g38180 |
| GGAATTATGGTCCCTGCTCA (SEQ ID = 655) | GCAAAGGGAGCATTAAACCA (SEQ ID = 656) | AW164518 | Glyma11g00640 |
| TCCTGATGGGAAAAGACCAC (SEQ ID = 657) | CTTGTCAAAGCTTTCGAGGG (SEQ ID = 658) | S15930971 | Glyma11g10310 |
| AACCCTTCTGATCCCGATTC (SEQ ID = 659) | ATTTGTGTTACAAAGGCGGG (SEQ ID = 660) | S5931556 | Glyma13g17760 |
| GCTGATGCTGGAACTGTGAA (SEQ ID = 661) | AACGCTTGACAAGGAGAGGA (SEQ ID = 662) | TC228853 | Glyma15g07590 |
| CTTCCAAAAGCCGTGCTAGT (SEQ ID = 663) | ATACGACACCTCGGATCTGC (SEQ ID = 664) | S4878382 | Glyma15g10370 |
| AGGCTGATCCATTTGGTTTG (SEQ ID = 665) | CATCGATGATCCAGCACTTG (SEQ ID = 666) | S4884795 | Glyma16g08450 |
| CCGTTCCTGATCTCGTTGAT (SEQ ID = 667) | GTTGAAGCACATCCACATGC (SEQ ID = 668) | AW471580 | Glyma04g00340 |
| CGTGAAAATGCAAGACTCCA (SEQ ID = 669) | CACTGCATTCCCAACTTGAA (SEQ ID = 670) | BQ610340 | Glyma01g01120 |
| AGGTGAGTCTGAGCCAGGAA (SEQ ID = 671) | GAAACCCAGTAGCCATCTCG (SEQ ID = 672) | BM887031 | Glyma07g04780 |
| GCTTCACTGTTTCTTTGTCACAC (SEQ ID = 673) | CCGTGCACATGGAACATAA (SEQ ID = 674) | CA938763 | Glyma14g37230 |
| TTCTGCATCCTCTGATGGAA (SEQ ID = 675) | TCAGGATTCAGGTTCATTGGA (SEQ ID = 676) | BG881491 | Glyma14g37230 |
| GCTGCGCAGGTAATCATTCT (SEQ ID = 677) | CTAGGCCATTGCTTGCTCA (SEQ ID = 678) | S21566814 | Glyma06g08610 |
| AAAACCGCCATTTTGTGTTT (SEQ ID = 679) | CGAAGGAGAGAGACAGAACGA (SEQ ID = 680) | S5014530 | Glyma01g29420 |
| TGAGGGCCGTTTTGAGATAC (SEQ ID = 681) | AGACCGACATTCCACCAGTC (SEQ ID = 682) | S4895927 | Glyma01g34410 |
| AAAGATCAATTCTGCGGGG (SEQ ID = 683) | ATTGTCGTACAACTGCGTCG (SEQ ID = 684) | S5076242 | Glyma03g07420 |
| CGCATGTCATTTCTGTTGCT (SEQ ID = 685) | GATGGAACCAGATGCAGACA (SEQ ID = 686) | BG316001 | Glyma03g41230 |
| CACTGATGAGGTCTTTGTGGC (SEQ ID = 687) | AAATAAACGTGGCCAACTGC (SEQ ID = 688) | TC214989 | Glyma05g01640 |
| AAGACCATCGAAATGGTTGTG (SEQ ID = 689) | TTTCCCTAGGAGCAACGCTA (SEQ ID = 690) | CD393873 | Glyma05g28090 |
| TAGCCTCATCCATTTTTGGC (SEQ ID = 691) | ATTGCAGAAGGGTGGTTGTC (SEQ ID = 692) | S15937116 | Glyma06g10400 |
| GGATCTCGCGAAACCGTTA (SEQ ID = 693) | AGCCTAAGCCTCTCCACCTC (SEQ ID = 694) | S4932942 | Glyma06g39800 |
| GTTGCTGCTGCCTATGACTG (SEQ ID = 695) | AACCGTTGTGTCCGGATTAG (SEQ ID = 696) | S4950242 | Glyma07g18500 |
| CTGAGGAGGTGGCTCAGAAC (SEQ ID = 697) | GCAGGTGATGTTGTGCAGTT (SEQ ID = 698) | S4932151; | Glyma08g01720 |
| | | S4932199 | |
| AATGACATTTTGCTCTGGGC (SEQ ID = 699) | AGTACGTTTGTCCTCGCTGC (SEQ ID = 700) | S5128657 | Glyma09g08690 |
| TAAAGCCAATCATGACACCG (SEQ ID = 701) | TTTCAGGGAAAGGAGCTGAA (SEQ ID = 702) | S5933258 | Glyma09g28080 |
| ACTTTTGTTATGGCCAACCG (SEQ ID = 703) | CGTCACCGTACTCTCGTTCA (SEQ ID = 704) | CF807678 | Glyma10g31020 |
| AGAAAGGCCCGTTGGACTAT (SEQ ID = 705) | AAGTAGCCAAACGGCAAAGA (SEQ ID = 706) | S4912433 | Glyma13g40560 |
| TGTCTTCTCTTCCACCACCC (SEQ ID = 707) | CCATCCTGCCGAAGTAAGAA (SEQ ID = 708) | S4912357 | Glyma17g11420 |
| GCCGATCCAAATCGTCTTTA (SEQ ID = 709) | GCAAAAGGGATTCTCAAAGC (SEQ ID = 710) | S4883295 | Glyma17g36490 |
| GTTGGCTACAATGCCACTCC (SEQ ID = 711) | AAGCCACGTCCTGGAAATC (SEQ ID = 712) | S21567638 | Glyma18g04060 |
| AATGGCTGCAAAATACCGAG (SEQ ID = 713) | ACTCAGACCCCAAATGCAAA (SEQ ID = 714) | S4863794 | Glyma18g46470 |
| ATTTCAACATCCTTCAGCCG (SEQ ID = 715) | AGTGCAAAGTGGGGTGATT (SEQ ID = 716) | S4995230 | Glyma19g32390 |
| CTTTTCCCCCAAATTTCGTT (SEQ ID = 717) | AATCATGAACCCCTGCAAAG (SEQ ID = 718) | CA785033 | Glyma08g32320 |
| GCAACTCTTCCAAGGCATTC (SEQ ID = 719) | TCCTCTGCCTATGGACAAGC (SEQ ID = 720) | CD418002 | Glyma09g36500 |
| TAAAAGAAGACACGGCACCC (SEQ ID = 721) | GGAGTTTGTGCAATGTGTGG (SEQ ID = 722) | S15851442 | Glyma20g27960 |
| GCCCTACAATCGAAGGGAAT (SEQ ID = 723) | TGATGGCCTTGTAGCCTAATG (SEQ ID = 724) | BI969358 | Glyma05g26040 |
| CAATATCTGCCAGGGCTTGT (SEQ ID = 725) | AAGAGTGCCTTTGAGGCAGA (SEQ ID = 726) | S22951692 | Glyma12g01050 |
| TCAAGATTTGTTCGGCCAGT (SEQ ID = 727) | CCGCCATCAGGACATCTAAT (SEQ ID = 728) | AI736779 | Glyma17g23500 |
| CTCTCCCTCCAGATGTCAGC (SEQ ID = 729) | TGGCTTAACCTTCGTTCCAC (SEQ ID = 730) | BE612133 | Glyma18g42790 |
| TCCAAACATCCTTTTCCGTG (SEQ ID = 731) | GTGTGAGGGGAAAAACATGG (SEQ ID = 732) | S4992234 | Glyma06g19840 |
| TTTGGTCAAACATGCAGAGG (SEQ ID = 733) | GAGACCAATGCCTTCCAAAA (SEQ ID = 734) | BI700659 | Glyma10g09410 |
| TTCGATCGAGGAACTGAGTG (SEQ ID = 735) | AGATGGTTCAGCAAAGCAGC (SEQ ID = 736) | TC230461 | Glyma12g09860 |
| TATCACTTCCAAACGCCCTT (SEQ ID = 737) | TTCTGAAGGGAAGACATGGG (SEQ ID = 738) | S23069339 | Glyma17g10130 |
| CGGGCTTCTATCGTGTCATT (SEQ ID = 739) | CTGATTACATGGGAGCACGA (SEQ ID = 740) | S4901375 | Glyma02g44220 |
| GAGGCCACAGAAGACAGTCC (SEQ ID = 741) | GATCCTGCCGAATGAAGTGT (SEQ ID = 742) | S4910851 | Glyma13g03660 |
| AAGACTGCCAGTTCACAGCC (SEQ ID = 743) | CAAGAGATCTTCTTCTGCGAATG (SEQ ID = 744) | S5035170 | Glyma13g03700 |
| GAAGCACAAATGGGTGGAGT (SEQ ID = 745) | TCAGGTGCTGGTAGTTGTGC (SEQ ID = 746) | CA819903 | Glyma13g41750 |
| TATTGGAGCTTGAGCCGCTA (SEQ ID = 747) | TCCATCCGAGACAATGATGA (SEQ ID = 748) | S4966677 | Glyma13g41750 |
| ACCTTCTCAGCAGCTTCGC (SEQ ID = 749) | GCTCCCTGCAAATTGTCATT (SEQ ID = 750) | S4876928 | Glyma20g12250 |
| AATGCAAAAGAGTCCTTCGG (SEQ ID = 751) | GCTTGACTTTGTTGTACCATTCC (SEQ ID = 752) | BG239314 | Glyma04g40150 |
| ACCACTTCCTCAGGACAACG (SEQ ID = 753) | TACACTTACACCCCACCCGT (SEQ ID = 754) | S21537202; | Glyma02g43240 |
| | | TC219068 | |
| TGGGCTAAGATCCCTTCCTT (SEQ ID = 755) | ATCCAAAGGAGCAGAAAGCA (SEQ ID = 756) | TC225486 | Glyma03g42450 |
| AGGTGTCCTTTGCCTTGTCA (SEQ ID = 757) | CAGCAGCCAAGATTGTTTCA (SEQ ID = 758) | S4882789 | Glyma03g42450 |
| CGGAGTTGATCACTGGGATT (SEQ ID = 759) | TCCAGAAAACAAGCCGAGAT (SEQ ID = 760) | BI468894 | Glyma03g42450 |
| GCTCTGGACAATGGACATCA (SEQ ID = 761) | TAAACAAATCCCGAATGCAC (SEQ ID = 762) | S4882586 | Glyma07g03250 |
| CCGAAATCGGTTTGACGTAT (SEQ ID = 763) | GAACGTGACAAAGGGGAAGA (SEQ ID = 764) | S18957277 | Glyma17g36500 |
| GATGGTTGTGATGGGGAAAC (SEQ ID = 765) | TTATGCAATGAGCAATCCCA (SEQ ID = 766) | BM731530 | Glyma11g07840 |
| AGGGCTTAAGCTTTTCGCAC (SEQ ID = 767) | TTGCGTGGATCATATCCTTTC (SEQ ID = 768) | TC212659 | Glyma11g08780 |
| GACTTGCTGGTGGTGGAAAT (SEQ ID = 769) | TCATCATTTCTCTGGGAGGG (SEQ ID = 770) | BE330095 | Glyma18g05080 |
| GTTTTGCCACGTGAAATCCT (SEQ ID = 771) | CGGTGCAGTTAAGCCAGTTT (SEQ ID = 772) | BU544833 | Glyma01g38360 |
| GCTGCAGCATGAAAATCAAA (SEQ ID = 773) | GGCGGACTACACATAGTGGG (SEQ ID = 774) | S23062201 | Glyma02g47640 |
| AGGCTGCATTCTTGGCTAAA (SEQ ID = 775) | ATTATGCCTTTCCCCATTCC (SEQ ID = 776) | CD405336 | Glyma03g03760 |
| TACCCTTACCAACCCCATCA (SEQ ID = 777) | GTGGGGGAGAAGGAGTAGGA (SEQ ID = 778) | BU926447 | Glyma05g22460 |
| GCTTCTTGTCATCTCTGGGG (SEQ ID = 779) | ACGTCCCCATTCTTTCACAG (SEQ ID = 780) | S5145856 | Glyma07g39650 |
| CGTTCACGTGATTGATTTCG (SEQ ID = 781) | AGTCGGAAAACCGGAGGAC (SEQ ID = 782) | CF808358 | Glyma08g10140 |
| CCGAGTCGCGGTTAAAGTAG (SEQ ID = 783) | TAACACAAGCAGATGCGACG (SEQ ID = 784) | S4911235 | Glyma10g37640 |
| TCCACATTTGAAAATCACCG (SEQ ID = 785) | CCAACTTTTCTGCCTCCTCA (SEQ ID = 786) | BU764181 | Glyma11g01850 |
| TCATCAAATCTGACGGTTGC (SEQ ID = 787) | TGGTCGAAGAGAATGGTTCC (SEQ ID = 788) | BU547766 | Glyma11g10220 |
| CTTCCCTTCGAGTTCTTCCC (SEQ ID = 789) | GATTGCCTCGTTAGGTCGAA (SEQ ID = 790) | S5137708 | Glyma11g10220 |
| AATGCTCCTTTCTTTGCCAC (SEQ ID = 791) | AACCTCCATTCGTTTTCACG (SEQ ID = 792) | S5087855 | Glyma11g14740 |
| ATTCCTGGCATAGCAGCCTA (SEQ ID = 793) | GGCGCTTGTTGATGTTGTTA (SEQ ID = 794) | S4996626 | Glyma11g33720 |
| TCCCAAGGTACAACTCGGAC (SEQ ID = 795) | TCCAGTCTTTTCGACTCGCT (SEQ ID = 796) | S23071313 | Glyma11g33720 |
| GCAGGCATCAGAGCAACATA (SEQ ID = 797) | ATTTCGACTCCGATACTGCG (SEQ ID = 798) | S19676947 | Glyma14g01020 |
| TTCTCAAAGAATTGCGGCTT (SEQ ID = 799) | GGAGGTTCCTTGCATCTCAA (SEQ ID = 800) | BU761164 | Glyma14g27290 |
| AGCCAAAGCTCCACATCATC (SEQ ID = 801) | TGAGGTGTCTCATCGTTTCG (SEQ ID = 802) | S21568820 | Glyma15g03290 |
| TCTCTTAGCCACCAATTCCG (SEQ ID = 803) | AAGATTGATGTGTGGAGGGC (SEQ ID = 804) | BU547981 | Glyma15g15110 |
| GCGTGGTGGATTTTGAGATT (SEQ ID = 805) | TCCTTTTTCTGCTACGGCTG (SEQ ID = 806) | BU763373 | Glyma16g29900 |
| TGGCTCTGGCTCAATTCTCT (SEQ ID = 807) | GGGAATTGGAGGAGGATGAT (SEQ ID = 808) | S15849261 | Glyma17g14030 |
| TTTATCCTCTTGCTGCCTCG (SEQ ID = 809) | GGTTGAACTTGTTCGAGTGGA (SEQ ID = 810) | BI944140 | Glyma18g04500 |
| AAAAACCCCAACCAAAGTCA (SEQ ID = 811) | ACACGGGAAGAGTGGTGAAT (SEQ ID = 812) | S23068790 | Glyma20934260 |
| TTTGTGAGGGCATCTGTGAG (SEQ ID = 813) | CATCTTGGGGCTCAGAACAT (SEQ ID = 814) | BU549908 | Glyma05938580 |
| CTTCTGGGGGATGGATTTTT (SEQ ID = 815) | GCCCTTTCAGTGACATCTCC (SEQ ID = 816) | BI945044 | Glyma20g30650 |
| CCATTTTCCATTGGTTGGAC (SEQ ID = 817) | GCCAATCCTATTTGGGATGA (SEQ ID = 818) | S21538571 | Glyma01901990 |
| CTCGCCTCAAGGAGTCAAAG (SEQ ID = 819) | AAAGATTACGTGGCGAGGTG (SEQ ID = 820) | S5146776 | Glyma01g39260 |
| CTAATACGGTGACGGTGGCT (SEQ ID = 821) | CCAGCAATCGGAGATGAGTT (SEQ ID = 822) | S5146735 | Glyma01g42640 |
| AAATGAGGCTGCAAAAGCAT (SEQ ID = 823) | GATGCAATGGCAGAAGGAAT (SEQ ID = 824) | BM271159 | Glyma01944330 |
| AACCCAACACGACTCCACA (SEQ ID = 825) | GCACGAGGCTAGGAAGAGAG (SEQ ID = 826) | CD403874 | Glyma03929190 |
| TCTCTTGGTCATCATGGAACAT (SEQ ID = 827) | TTTACGAAGTCCCTTGCACC (SEQ ID = 828) | TC210199 | Glyma05920460 |
| AAATAATTGGCGTTTGGCTG (SEQ ID = 829) | ATCCCATCAGAAGCAACTGG (SEQ ID = 830) | TC208761 | Glyma05934450 |
| CTGCGTTTACACGGATGAAA (SEQ ID = 831) | CTGGCTCCTCCTAAGTGCAT (SEQ ID = 832) | S4861816 | Glyma06904390 |
| GCGGTGCAGTCTGATTACAA (SEQ ID = 833) | TCTCCACCCTTGAGAAAACG (SEQ ID = 834) | BGT54271 | Glyma08906130 |
| CAACTACCGAGCAAACCCAT (SEQ ID = 835) | CATGCCCAACTCAAAGTGTG (SEQ ID = 836) | TC219635 | Glyma08911460 |
| TGGTGTTCCAGACGATGAAG (SEQ ID = 837) | TCTCACCAAACCCTTCCAAC (SEQ ID = 838) | S23072015 | Glyma10g38240 |
| CATTGAACTAGCTGGGTGACAG (SEQ ID = 839) | TTGGGCCAAGAAATTGAGTC (SEQ ID = 840) | BI699405 | Glyma10938930 |
| ATTCCGCTTCATTGTATGGC (SEQ ID = 841) | AAGTTGACGGACGAAACTGG (SEQ ID = 842) | S5146771 | Glyma11902800 |
| GATTGGCCAACACATTGACA (SEQ ID = 843) | GTGAGGGTTTTGAGGGTGAA (SEQ ID = 844) | S4980779 | Glyma11g13600 |
| TTGGCTTAGGAAGTTTGGGA (SEQ ID = 845) | GGTTGACCAGCTTGACCATT (SEQ ID = 846) | TC212225 | Glyma13g21490 |
| GAAGCTTGTGTTCGTGCGT (SEQ ID = 847) | GCGGACATATGGATAGGAAAA (SEQ ID = 848) | TC221978 | Glyma14g09190 |
| GAAGCAGTGACATGTGGTGG (SEQ ID = 849) | ATCTTGCTCAGAAACGGAGG (SEQ ID = 850) | S5146772 | Glyma14911030 |
| TCAAAGGGTGTGCAACTGAC (SEQ ID = 851) | TTTCGGATTCCCTACAGCAC (SEQ ID = 852) | TC206227 | Glyma16g32070 |
| TCACTATAGGGAATTTGGCCC (SEQ ID = 853) | TTCAACACTACCCTCAATGGC (SEQ ID = 854) | S4937910 | Glyma16932070 |
| GCTTTCACTCATCTCAGCCC (SEQ ID = 855) | AAGGCCAATGTTGTTTGGAG (SEQ ID = 856) | S21566681 | Glyma19g31940 |
| CCCCATGTCTGACCAAGACT (SEQ ID = 857) | GTGGATCCCAAACCACAAAG (SEQ ID = 858) | BE348040 | Glyma19g34210 |
| TCGGTGTACTAATCAGATGCAGA (SEQ ID = 859) | TCCATTTCCGAGGGCTACTA (SEQ ID = 860) | TC216962 | Glyma04g10340 |
| TTTCTTGATCACAGACCCTCT (SEQ ID = 861) | TCCCTGAAGAATAGCACCCA (SEQ ID = 862) | S4876002 | Glyma04g16180 |
| GCAGGGCAGTATTTACGCAT (SEQ ID = 863) | TTTGTGGTAACTGCGCTTTG (SEQ ID = 864) | CD395272 | Glyma03g34850 |
| TGGGCATTCTCCCACTTATC (SEQ ID = 865) | TGGCTGCATGGCATATAGAA (SEQ ID = 866) | S7107295 | Glyma05g32600 |
| TTGCATGCACACTTGCAATA (SEQ ID = 867) | GCAGCTCACTTCCAAGTTCC (SEQ ID = 868) | CD408414 | Glyma05g32600 |
| TGCAGAAGGAGCAGAAGGAT (SEQ ID = 869) | GTAACTGAAACGGCTCCCAA (SEQ ID = 870) | AW509447 | Glyma17g13000 |
| GATCGTGAGAAGGAAGCCTG (SEQ ID = 871) | CTTCAATGAGCGGGGTTCTA (SEQ ID = 872) | BE191307 | Glyma13g04790 |
| GTGTTGGTTTCTCAGGCGTT (SEQ ID = 873) | CAACACTCTCTGGAGCATCG (SEQ ID = 874) | AW132814 | Glyma02g41830 |
| CCACTCATCAGCTACCCCAT (SEQ ID = 875) | TAATTTGATGTTCCCTCGCC (SEQ ID = 876) | S23068139 | Glyma07g19420 |
| ATGGTTGCATCTCAGCCTCT (SEQ ID = 877) | GAGACTGTCTGACCAAGGGC (SEQ ID = 878) | BU764116 | Glyma08g09700 |
| CTCAATGCCTTCGGCATAAT (SEQ ID = 879) | GGAAGGCAATCGTGGTTAAA (SEQ ID = 880) | S5059806 | Glyma08g09700 |
| ACAAGGGAAGATGGTGATCG (SEQ ID = 881) | ATTGCCATCGTTGTGTTCAA (SEQ ID = 882) | AW703667 | Glyma13g25640 |
| ATCATTGTAGGTTGGCTGGAG (SEQ ID = 883) | ATGGAAAAACTGGCGCGAA (SEQ ID = 884) | S4901892 | Glyma07g04200 |
| GATGACCGAAAGGTTGGAAA (SEQ ID = 885) | TGGGTGGTCTTTTAGGCTTG (SEQ ID = 886) | CF808586 | Glyma03g08270 |
| TTTTGTGCTGGTGAAAGGAA (SEQ ID = 887) | TTAAGGGTCCATGCCAAAAG (SEQ ID = 888) | S4862200 | Glyma03g08270 |
| TAACCGCTCCTGTTCGACTT (SEQ ID = 889) | GCCGAAGGCACATCTAGTTC (SEQ ID = 890) | S23070980 | Glyma06g48010 |
| GCAGGAAGCGACACGTTAAT (SEQ ID = 891) | TCTACCCTTGATCCAGTGCC (SEQ ID = 892) | S4993820 | Glyma17g14520 |
| TCAGCAATTTCAGCTCATGG (SEQ ID = 893) | TTCCGTCGGTTCCATATTTC (SEQ ID = 894) | S5006690 | Glyma18g46540 |
| AGTCAATTCCCGAACCACAG (SEQ ID = 895) | ACTGAGGGAGTCAAGAGCGA (SEQ ID = 896) | S15853197 | Glyma01g01850 |
| CTGGGCCATTGTTGATTTTC (SEQ ID = 897) | GAATAACGCAGCCAGAGGAC (SEQ ID = 898) | BM893519 | Glyma01g01850 |
| TGGTTCTGAGCTTGAAGTGC (SEQ ID = 899) | CAGGTGGAAGACCAAGCAGT (SEQ ID = 900) | S23068795 | Glyma02g02290 |
| TGTTGTAGTCACCTGCTGGC (SEQ ID = 901) | GCTTTTGATGGGCTGCTATC (SEQ ID = 902) | CF807495 | Glyma02g10410 |
| CAGGTCTAATGGTGGGTGCT (SEQ ID = 903) | TGCAAGTGAATGTCGGGATA (SEQ ID = 904) | S5142660 | Glyma02g42200 |
| GCAACTGAACTTCCAAAGGG (SEQ ID = 905) | ATTCATTGGTGGGAATTGGA (SEQ ID = 906) | BM308002 | Glyma03g01000 |
| GTTGTCCAAGGAACAGGCAT (SEQ ID = 907) | CCAAAGCTTGCTTTTGCTTC (SEQ ID = 908) | AI795005 | Glyma03g26700 |
| CCAACAATTGGGAATGATCC (SEQ ID = 909) | AGGAAGTGTTCGAAGAGCCA (SEQ ID = 910) | BU765815 | Glyma03g36070 |
| TCATTCAATAATCAGCTGCG (SEQ ID = 911) | GATGAAGGGGTTTGAGTTTGA (SEQ ID = 912) | S4936521 | Glyma04g04310 |
| TTGACTTTTCATTGACCCGA (SEQ ID = 913) | TCACTCGATTCGACTAGCCA (SEQ ID = 914) | S4865673 | Glyma04g04310 |
| AAGGAAAGGGAGGGAACAGA (SEQ ID = 915) | AGGGATACTGAAAACCGCCT (SEQ ID = 916) | S22953100 | Glyma04g06810 |
| CCTTCTGGTTTTCGCATCAT (SEQ ID = 917) | CAAGTGCAGAAGCCAAATCA (SEQ ID = 918) | TC206511 | Glyma04g09000 |
| TCCTCCGAGAGAAGGAACAA (SEQ ID = 919) | CGAGTTTCTTGGCTAGGCTG (SEQ ID = 920) | BM887093 | Glyma04g40960 |
| ATCTTTCCCGTTTTCTGGGT (SEQ ID = 921) | CCCTCGTTCTCTGTGTGGTT (SEQ ID = 922) | S4979247 | Glyma05g01060 |
| TGAACCTGTGGTTTCGATGA (SEQ ID = 923) | ACGCAGGGTTTTTCATTCAG (SEQ ID = 924) | S4872528 | Glyma05g01400 |
| GAAACACGGTCGTTCCTGC (SEQ ID = 925) | TCGTTTTCCGCTCACGCAC (SEQ ID = 926) | CA783321; | Glyma05g04990 |
| | | S6669218 | |
| CGTCAGGTTTCGAATTGGTT (SEQ ID = 927) | CGTCGTTTTCTTGCTCCTTC (SEQ ID = 928) | S4981726 | Glyma05g37550 |
| ATTTTGTGTCAGGGCTGAGG (SEQ ID = 929) | TGCCTCGCAGTTATCTTGTG (SEQ ID = 930) | CA799411 | Glyma06g01940 |
| CCGAGAGGAAGATTTGGCTA (SEQ ID = 931) | TTCCATCTGCTTGGTCTTCC (SEQ ID = 932) | S4896994 | Glyma06g20230 |
| TTCCCCTAGAAGCTCTGCAA (SEQ ID = 933) | AGGTCTTCGCTTGATGAGGA (SEQ ID = 934) | AW395625 | Glyma06g44290 |
| TCATCAACGGTACTGGCTCA (SEQ ID = 935) | CCAGTGACGTTGGACTGAGA (SEQ ID = 936) | CF808925 | Glyma07g01950 |
| CGAACGTTCTGGATGGACTT (SEQ ID = 937) | CGACGAAGCATGTGAAAATC (SEQ ID = 938) | BG041551 | Glyma07g02220 |
| ATTGCCATTTTCAAGCCATC (SEQ ID = 939) | TGGAGCAACAGTACGCCATA (SEQ ID = 940) | S21539727 | Glyma07g06460 |
| ATCCCTGTGCAGTTGATTCC (SEQ ID = 941) | CACTGATTGAATGGGGTGTG (SEQ ID = 942) | TC233702 | Glyma08g03160 |
| GCAATGCTAATCTAATGGCACA (SEQ ID = 943) | TTGTCACACCAACAACGAATG (SEQ ID = 944) | S22951609 | Glyma08g13110 |
| TTATCGGGAAGATGGTCCAC (SEQ ID = 945) | AAGAGCAGGATTTGCAGCAT (SEQ ID = 946) | BM528044 | Glyma08g41330 |
| ATGCAGTTTGTGGTGATGGA (SEQ ID = 947) | TAGAGCATGGGATGGGAAAG (SEQ ID = 948) | S5146881 | Glyma09g01000 |
| TGAACCATATCTAGAGACTACTACT (SEQ ID = 949) | AGCATACTTCATACATAGGGCA (SEQ ID = 950) | S5075763 | Glyma09g02750 |
| TCTGCTTTAATTGCAGCCCT (SEQ ID = 951) | GCGACACCACTTCCCTTTTA (SEQ ID = 952) | S4867945 | Glyma09g12820 |
| TAATGAACCCCGGGTATGTC (SEQ ID = 953) | GGGGAGACTTTGTAGGGAGG (SEQ ID = 954) | BI469367 | Glyma10g10040 |
| CACACATCACACGAGCAGAA (SEQ ID = 955) | GGTGTAAGTGGCAGTGGCTT (SEQ ID = 956) | S21567823 | Glyma10g28820 |
| CACACATCACACGAGCAGAA (SEQ ID = 957) | GGTGTAAGTGGCAGTGGCTT (SEQ ID = 958) | BU548090 | Glyma10g28820 |
| AAGTCTCTGTGCTCTTGTTGGA (SEQ ID = 959) | TGATGATAGGATGGGCACTA (SEQ ID = 960) | S4883516 | Glyma10g38280 |
| CAGCTGAAGGCGGAGATAAC (SEQ ID = 961) | TGAGCATCGATGAGTGGAAG (SEQ.ID = 962) | TC217986 | Glyma11g02960 |
| ATCGTTGTCTTCTTCGCTGG (SEQ ID = 963) | TCCACCTCCACCTTGTTGAT (SEQ ID = 964) | AW757139 | Glyma11g06640 |
| GCACCGACCCTTATATTGGA (SEQ ID = 965) | ATCTTGGGTGTCCAAAGGTG (SEQ ID = 966) | S4916693 | Glyma12g33430 |
| ACTTCAACATCCCTCAACGC (SEQ ID = 967) | GGAAAACGACATTGAACGCT (SEQ ID = 968) | S5115730 | Glyma13g05270 |
| CTGAACTTGCTTTTCGAGGG (SEQ ID = 969) | TCATACAGTTCGTCCGGTCA (SEQ ID = 970) | BG239618 | Glyma13g23890 |
| TTGGCCCAAATCTCCATAAG (SEQ ID = 971) | CTGGCCGGGTTAAAAAGAAT (SEQ ID = 972) | S23067438 | Glyma13g44930 |
| TTTCTCCACCTCATCATCCTG (SEQ ID = 973) | CGGAGGATCCAATTCCAAGT (SEQ ID = 974) | BQ253856 | Glyma14g09310 |
| GAGAGTTGCACTCTGCGGAT (SEQ ID = 975) | CATAAACCAGAGGAAGAGGCA (SEQ ID = 976) | BE658510 | Glyma14g10430 |
| CCGCCATCTTTAACTGGAAA (SEQ ID = 977) | TGTTGGTCCATGTCTGGAAA (SEQ ID = 978) | S5146505 | Glyma15g04700 |
| GGCCACAAATTCTACATCCA (SEQ ID = 979) | TGGAGGGTGAGTCATTGTTGT (SEQ ID = 980) | S5874971 | Glyma15g42380 |
| AGGCTCAAGCCTTGTCTCTG (SEQ ID = 981) | ACCACCCCATCAAGATCAAA (SEQ ID = 982) | S23069184 | Glyma16g02390 |
| TCCCTTTTTCATCCAGAATCC (SEQ ID = 983) | CCCTTTTAATGCATGCTCGT (SEQ ID = 984) | S4934495 | Glyma17g11330 |
| GTTTCACGGAGGAGCAAGAG (SEQ ID = 985) | CGGTGTCGAGGAAATTCTGT (SEQ ID = 986) | S5055444 | Glyma17g11330 |
| GGGGTTACACACCTACACGG (SEQ ID = 987) | CCACCACTGATCTTGAGGGT (SEQ ID = 988) | S23064210 | Glyma17g15380 |
| CAAAAACCAAAGAAGAGTTGCC (SEQ ID = 989) | CACTAGCTATGTAGTTCATAAGACG (SEQ ID = 990) | S4898544 | Glyma17g16930 |
| GCCGCCAGAAAGAAACTTAG (SEQ ID = 991) | GCTTCGCCAAAGCTTGAATA (SEQ ID = 992) | TC205125 | Glyma17g16930 |
| TCTTCGTCGCCAAATTCTTT (SEQ ID = 993) | CAGCGACTGAAACAGAGCAG (SEQ ID = 994) | S4904898 | Glyma17g17540 |
| TGGCTCTTTGAGCACTTCCT (SEQ ID = 995) | CAATTTGCCACCTGGTTTTT (SEQ ID = 996) | BM568090 | Glyma17g37260 |
| GAGTCTGCAGGCCTCGTTAT (SEQ ID = 997) | AACGAAGCCTTACGAAAGCA (SEQ ID = 998) | S23062061 | Glyma18g01830 |
| CGGAACCAGAAACTACAGGC (SEQ ID = 999) | ATTGCTCCATGAACCCTCAG (SEQ ID = 1000) | BE211253 | Glyma18g49290 |
| GAAGCGGTCCATGTCGTTAT (SEQ ID = 1001) | GAAGACCCCATCATCGGATA (SEQ ID = 1002) | S5118421 | Glyma18g49290 |
| TTCTTCAGATCCACCCGTTC (SEQ ID = 1003) | CACACGTTCCATACCCAGTG (SEQ ID = 1004) | BM954422 | Glyma19g33100 |
| GAGACTGGCTCTCTGGGTTG (SEQ ID = 1005) | AAGACAGGGGAATACAGGGG (SEQ ID = 1006) | BE347092 | Glyma20g26700 |
| TGCACCCAGTTGTCATCAAT (SEQ ID = 1007) | TTGAGCAGCATCCAATCAAG (SEQ ID = 1008) | S15850208 | Glyma05g29040 |
| GGTTTTGGCCAGTGGAATTA (SEQ ID = 1009) | CATCAGGGACTCCTTTTCCA (SEQ ID = 1010) | S5050877 | Glyma06g10660 |
| GTTGCAGATTGTGCCGTATG (SEQ ID = 1011) | CCCAGACTCACTTCTCTGGC (SEQ ID = 1012) | BI974743 | Glyma08g06460 |
| CGCCATTTTCTTTACCTCCA (SEQ ID = 1013) | GGAATTTGTGTCCCCTGAAA (SEQ ID = 1014) | BE820243 | Glyma08g06460 |
| GATGACTCCCCTGCTGAAAA (SEQ ID = 1015) | GCTTGCTACAGGGAAACACC (SEQ ID = 1016) | AW734397 | Glyma10g35350 |
| GTGGTTCCACCATTGCTTCT (SEQ ID = 1017) | AAAACTTGGGCATGTTCAGC (SEQ ID = 1018) | BI967222 | Glyma09g30330 |
| CCTGCGACTGCATTGAACTA (SEQ ID = 1019) | GAGAGTATCCGGCGTCACAT (SEQ ID = 1020) | S4916861 | Glyma04g04880 |
| TGAAAAGGGAGACGAATGCT (SEQ ID = 1021) | TGATTCTTGTACGGTGGCTG (SEQ ID = 1022) | S4994481 | Glyma04g05500 |
| AAGCGAAGGACTCAGACTCG (SEQ ID = 1023) | CGACGAGTAGAACGCAGTGA (SEQ ID = 1024) | S4913107 | Glyma04g05500 |
| GGAAACTGGTCATGGTAAGTAGAA (SEQ ID = | CCACCAGCTTGAGTCATGG (SEQ ID = 1026) | S15922397 | Glyma14g06800 |
| 1025) | | | |
| TCCTTGCCTTACGCTAGTCTTT (SEQ ID = 1027) | TGACAACAAGCTTCAAAGGAGA (SEQ ID = 1028) | TC208095 | Glyma14g12350 |
| GAAGGAATGTATCTGATGGGG (SEQ ID = 1029) | TTGTGTTTCAGAATATGGCCTG (SEQ ID = 1030) | S21568145 | Glyma14g12350 |
| AGGTTGCTTTAGTCTCCGCA (SEQ ID = 1031) | CCAAGGGAAAGAACAGGACA (SEQ ID = 1032) | TC204441 | Glyma17g35290 |
| AGTCGCCACGGAGATATGAT (SEQ ID = 1033) | TATGTGGTAGTGCGTGGGAG (SEQ ID = 1034) | S4877587 | Glyma17g35290 |
| TCACAAGCCTTGCACTTTTG (SEQ ID = 1035) | TTGGAATGGGTGGTGAATTT (SEQ ID = 1036) | S23064130 | Glyma18g03490 |
| CACGGGACATTCAACATCTG (SEQ ID = 1037) | TGCCATTGTTTATGCTCCAA (SEQ ID = 1038) | BM526782 | Glyma04g07460 |
| TCTCCACAAGTTCAAGCACG (SEQ ID = 1039) | ACCAGCAGCTCTGGGATTTA (SEQ ID = 1040) | AW508563 | Glyma04g07460 |
| TCTTTGGGTGGAAATCAAGG (SEQ ID = 1041) | CGTTTGATACAACTGTGCGG (SEQ ID = 1042) | S23061430 | Glyma10g18620 |
| CCTCTTTTGCCATTTGGGTA (SEQ ID = 1043) | TGAAACAGGATACAACAGGGG (SEQ ID = 1044) | S5084249 | Glyma17g30910 |
| GCATCACATGTCCCTCACAC (SEQ ID = 1045) | TTAAGGCTGAGCCGTTGACT (SEQ ID = 1046) | S5058162 | Glyma02g04710 |
| GCAAGCTCACTCGCTTTCTT (SEQ ID = 1047) | TAAGAAGACCAAAGGTCGGC (SEQ ID = 1048) | S5108603 | Glyma02g30990 |
| CCACGGAGAAGATTCGTGAG (SEQ ID = 1049) | TGCTTAAGCTCTCTCCATCAGA (SEQ ID = 1050) | BU549106 | Glyma04g02980 |
| AGAAGGTGTGGGAAACATGC (SEQ ID = 1051) | GCTGTTTTAGGCTAGCTGCG (SEQ ID = 1052) | BE058034 | Glyma04g42420 |
| ATTTGACTTCTGGGGAGCCT (SEQ ID = 1053) | GACCCCACAAGAGCAAGAAG (SEQ ID = 1054) | S21538617 | Glyma05g07380 |
| GACCCCACAAGAGCAAGAAG (SEQ ID = 1055) | ATTTGACTTCTGGGGAGCCT (SEQ ID = 1056) | TC208789 | Glyma05g07380 |
| GCATAAGATCCACTGCACCA (SEQ ID = 1057) | ACACGGCAGACACTTACAGC (SEQ ID = 1058) | S4889056 | Glyma05g28140 |
| TGGAGGGGAGTACGAGTCTG (SEQ ID = 1059) | TAGGATGGCTTGGCTGTAGG (SEQ ID = 1060) | S22336596 | Glyma06g02990 |
| GACGAAGAGGATTACGACGG (SEQ ID = 1061) | AGGCCGGACATTCAACTCTA (SEQ ID = 1062) | S4876998 | Glyma06g09870 |
| CGTGGTGATGAAATGGATCTT (SEQ ID = 1063) | GGAGTTGGGGTTCCTTCATT (SEQ ID = 1064) | S5062283 | Glyma06g22660 |
| GATACTCCAGAACGGGACGA (SEQ ID = 1065) | GCTATGCTGATGCTCAGTCG (SEQ ID = 1066) | S4891674 | Glyma06g48270 |
| ATGCTTTGGCCAATGTGAAT (SEQ ID = 1067) | TCTTCGTTGGCATGGTCATA (SEQ ID = 1068) | S5103646 | Glyma08g02930 |
| GAATGGATTCCGATGATTGC (SEQ ID = 1069) | TATGCAAGAGATCAGCACGC (SEQ ID = 1070) | S15850478 | Glyma08g07260 |
| TCAAGGGTTGAGTGTGCAAG (SEQ ID = 1071) | CGTGGTGACACGGTCTATTG (SEQ ID = 1072) | S21540484 | Glyma08g11110 |
| ATTCCTGCATTAGGGAACCA (SEQ ID = 1073) | AAGCAAGTTCCCCAGGCTAC (SEQ ID = 1074) | S5049230 | Glyma08g11110 |
| TTGTTGTGGTTTTGCAGCTC (SEQ ID = 1075) | CGAGGGTAGATTGGAGAAAGG (SEQ ID = 1076) | S4993992 | Glyma08g42300 |
| GTGCTGATGACAGAACGCAT (SEQ ID = 1077) | TGCGATCCATCCACAATTTA (SEQ ID = 1078) | S4992495 | Glyma11g07820 |
| AGTACGAGTTTTGCAGCGGT (SEQ ID = 1079) | GCTTCCTTTGTTGCCACATT (SEQ ID = 1080) | S23162106 | Glyma11g36890 |
| GTCTGTCAAGGCGAGAAAGC (SEQ ID = 1081) | CCGAAGCTCCTCAATCTGTC (SEQ ID = 1082) | S21691323 | Glyma12g17720 |
| CCTTGTGTGGAGTTGAAGCA (SEQ ID = 1083) | GGAGTGTGCCAATACAGGGT (SEQ ID = 1084) | BE610209 | Glyma13g07720 |
| CTACCAATCGCCAAGTCACA (SEQ ID = 1085) | CGTCCACGGCTAGAGAAAAC (SEQ ID = 1086) | S29966237 | Glyma13g29510 |
| AACCCTATTGAACACCCTTGA (SEQ ID = 1087) | TTCTGCATACACTCATGCAACA (SEQ ID = 1088) | S4884815 | Glyma13g33020 |
| TATTTCCTTTCGCAGGATGC (SEQ ID = 1089) | GCATTCAGGGATTCAAGGAT (SEQ ID = 1090) | S15853888 | Glyma13g33040 |
| GCTGAACACGAGAAAGCACA (SEQ ID = 1091) | TAACAGGGAAGAAATTGCGG (SEQ ID = 1092) | AW433203; | Glyma14g03100 |
| | | S4907367 | |
| CGGGTACGAATTTGCTTGAG (SEQ ID = 1093) | TTGCAGAGAAACCATAGGCA (SEQ ID = 1094) | S15940131 | Glyma16g13070 |
| TTGGAAAATTGGGAGTGAGG (SEQ ID = 1095) | ACCGGCATAAGATCCACAAC (SEQ ID = 1096) | TC231648 | Glyma02g38800 |
| TTCTTTGGGGGTTGAAGTTG (SEQ ID = 1097) | CCGCTCCAAGAAAAATTCTG (SEQ ID = 1098) | TC229785 | Glyma05g15170 |
| AGAGCTTGTGGAATTCCCTG (SEQ ID = 1099) | AGCATCCAATTCAAGGAACA (SEQ ID = 1100) | TC211088 | Glyma08g05110 |
| TTGGATTTGTGATGCCGTTA (SEQ ID = 1101) | CATCATAGGAAGGGAGGCAA (SEQ ID = 1102) | S4967171 | Glyma01g00600 |
| TTCTTTTCAAGCAACGCTGA (SEQ ID = 1103) | AGTAGTGGGCACTCGTCACC (SEQ ID = 1104) | S23062403 | Glyma01g04530 |
| ATCAGCAGTCAAGAGCACCA (SEQ ID = 1105) | CAAATTGCAGACACGATGCT (SEQ ID = 1106) | AI900277 | Glyma01g05190 |
| GGTTCTTGGACTGTTGACCG (SEQ ID = 1107) | GAAATGCAAGTAATTTCCCCC (SEQ ID = 1108) | TC224483 | Glyma01g26650 |
| ACACCTTTGTCCACCGATTC (SEQ ID = 1109) | TCCGTCCACCAAGAAAAATC (SEQ ID = 1110) | BU578344 | Glyma01g40220 |
| TGCCGAATTCAATGATACCC (SEQ ID = 1111) | TGGCATGCATTTCTGGTATG (SEQ ID = 1112) | S5143215 | Glyma02g00820 |
| CTGTCAACGGAAAGTGCAGA (SEQ ID = 1113) | CTGCATCACCAAAACCATTG (SEQ ID = 1114) | S34273499 | Glyma02g01300 |
| GCCACTCCTTTCAGGAAGTT (SEQ ID = 1115) | CCCAAGTTCTTATGTGAATACCC (SEQ ID = 1116) | S23063261 | Glyma02g39000 |
| TGCATTTACTAGATCACGGGG (SEQ ID = 1117) | TGGAATATCTGCAACAGGATG (SEQ ID = 1118) | TC227422 | Glyma02g40800 |
| GCATCGAGAAGGAAAACGAA (SEQ ID = 1119) | TTCCTCTGATTTTTCCCCAG (SEQ ID = 1120) | TC221184 | Glyma02g43280 |
| CGTTGTTCCTTTGGCAATTT (SEQ ID = 1121) | CTTCCATGCAGATGATGCAC (SEQ ID = 1122) | S5001333 | Glyma02g43280 |
| TAGGCACAGTTTCACATGGC (SEQ ID = 1123) | ATCCACCATCCCAGAATCAA (SEQ ID = 1124) | S23068701; | Glyma03g14440 |
| | | TC228909 | |
| GTTTGGCGTCTTGGTTTGAT (SEQ ID = 1125) | AAGAAGAGGCTGCCACAAAA (SEQ ID = 1126) | S23065855 | Glyma03g31980 |
| CTTGGAGGGTTATGTTCCCA (SEQ ID = 1127) | GTCTAAAACGAACGGGCAAA (SEQ ID = 1128) | S23068160 | Glyma03g38040 |
| GTTACTGGGAAGCAAGTGCC (SEQ ID = 1129) | TCAATTCCCAAGAAGAGAGCA (SEQ ID = 1130) | S4896043 | Glyma03g38410 |
| AGCAGTGGCAACAACAACAG (SEQ ID = 1131) | AGTTGAGGTGCTGGAAAGGA (SEQ ID = 1132) | TC211951 | Glyma03g38660 |
| CTTTTGCAGTAGCATCACCG (SEQ ID = 1133) | TGTGACATGGAACACACCAA (SEQ ID = 1134) | S34273417 | Glyma03g42260 |
| GCCATATGCAAATGCAGAAA (SEQ ID = 1135) | AGCAGCTGCAATAGCTGTCA (SEQ ID = 1136) | S34273457 | Glyma03g42260 |
| GCCGTTAAGAACCACTGGAA (SEQ ID = 1137) | GGAGGAGCAAGAGTCAATGC (SEQ ID = 1138) | S4873244 | Glyma04g03910 |
| TTCCCCTCTAATTCAACCCC (SEQ ID = 1139) | TCTCCTGTGAGGCAACTCCT (SEQ ID = 1140) | S4975581 | Glyma04g32690 |
| AAGCACTTACCCATGCGAAC (SEQ ID = 1141) | CTTGAGGGATCCACAGCATT (SEQ ID = 1142) | BI785347 | Glyma04g33210 |
| TCCTTTCTCTTTTGGTGGGA (SEQ ID = 1143) | GGGTCCGTACAAGGAACAGA (SEQ ID = 1144) | S4870629 | Glyma04g34720 |
| AGGACCTTTTCATTGGCCTT (SEQ ID = 1145) | ATCATCATGCTCTTCCGGTC (SEQ ID = 1146) | S4982467 | Glyma04g38240 |
| TTCTCCAGTGTTCCCGTTTC (SEQ ID = 1147) | TGCAGTTGGTTTCAGCACTT (SEQ ID = 1148) | S4910460 | Glyma05g04950 |
| TTTCATCAGGCAAAGCAATG (SEQ ID = 1149) | GCAGTGTCAGCTGCTTCATC (SEQ ID = 1150) | TC215913 | Glyma05g04950 |
| TAAATGAAGAGGGCCCATGA (SEQ ID = 1151) | CGTCGTGAATGGATAAGCAA (SEQ ID = 1152) | S34273496 | Glyma05g35050 |
| TGCAGTCTGGTTGCATAATAGC (SEQ ID = 1153) | CGTCGTTTTTCAGGCAAGAT (SEQ ID = 1154) | S4875209 | Glyma06g00630 |
| CACGAAATTTGGTCCCTCAT (SEQ ID = 1155) | GGGTAAGCTGATTGCACCAT (SEQ ID = 1156) | S4928297 | Glyma06g04010 |
| CCTGGAAGAACCGATAACGA (SEQ ID = 1157) | TGAGTTTGAGGGTCGATTCC (SEQ ID = 1158) | BM308450 | Glyma06g16820 |
| CAATGAGAACACCCCTTTTGA (SEQ ID = 1159) | CTCCAGAATGTGGTGGGAAT (SEQ ID = 1160) | TC233743 | Glyma06g45520 |
| CAGAATACAGCTCGTGCCAA (SEQ ID = 1161) | TGACCAAGTTTGGACCCCTA (SEQ ID = 1162) | BU549656 | Glyma06g47000 |
| GCCCCAAAGAGATCAACAAA (SEQ ID = 1163) | CCGCATCTCTTTAAACCTGC (SEQ ID = 1164) | S4891301 | Glyma07g04210 |
| TCAGCTGATAAGAATCAGACTTGT (SEQ ID = 1165) | TTTCCAAGCTGATAGAACGCT (SEQ ID = 1166) | S19677672 | Glyma07g05960 |
| AGTGGCAGTGCAATTCACAA (SEQ ID = 1167) | TGTCCAACCACCCTTAGCAC (SEQ ID = 1168) | TC231964 | Glyma07g15820 |
| TGAAGTGCATCATGCTTTGG (SEQ ID = 1169) | TCCTCCATCTTCTCCCTCCT (SEQ ID = 1170) | S25049562 | Glyma07g15850 |
| AATAGCTGGGAGATTGCCTG (SEQ ID = 1171) | GGGTCAATGCCTTTGCTAAT (SEQ ID = 1172) | S34273436 | Glyma07g33960 |
| AACCACATGATTGATTGCCA (SEQ ID = 1173) | TCTGGTTACTCGTAGCATCGC (SEQ ID = 1174) | S5011023 | Glyma08g04670 |
| TTACCACCTCAAGAGCCACC (SEQ ID = 1175) | AGCCGAAGCTCTCATACCAA (SEQ ID = 1176) | TC219749 | Glyma08g17400 |
| TGGTGCTCCAGCAACAACT (SEQ ID = 1177) | ACCCCAGTGATGAACCTTCC (SEQ ID = 1178) | S5144915 | Glyma08g40020 |
| GCTTTTGCTTTGCTTTGCTT (SEQ ID = 1179) | AGGGACACAGATCCGAGATG (SEQ ID = 1180) | BF598100 | Glyma09g02030 |
| TGTGTACCAAACGAATCCGA (SEQ ID = 1181) | TGGGAACATGATGGTGAGAA (SEQ ID = 1182) | S21538601 | Glyma09g03690 |
| CTTGGCATCTTTGTGTCCCT (SEQ ID = 1183) | CATTCTGGTGCTTTGTCCAC (SEQ ID = 1184) | S4898539 | Glyma09929800 |
| CTGCATCACCAAAACCATTG (SEQ ID = 1185) | TTCATCATCGGAAAGTGCAG (SEQ ID = 1186) | S5146038 | Glyma10g01340 |
| TGTCAAACCGCTTAACACCA (SEQ ID = 1187) | GTGCAAGATATTCCCCATGC (SEQ ID = 1188) | S4870840 | Glyma10g05560 |
| CAAGCTCGTCATTTTGCTCA (SEQ ID = 1189) | TCAAGCTACCGAACTCCCAT (SEQ ID = 1190) | S4995311 | Glyma10g06560 |
| AATCCCTTGAATTGGAACCC (SEQ ID = 1191) | TTCCAAGGACATCCAGAAGC (SEQ ID = 1192) | S23069233 | Glyma10g27940 |
| TGTGGTGATTCTCGTCCATC (SEQ ID = 1193) | GCTGCTGGAAACCTTTCTGA (SEQ ID = 1194) | BM893228 | Glyma10g27940 |
| AAAGATGTTGCTGCCGACTT (SEQ ID = 1195) | AGCACACACCTGTGGTCAGA (SEQ ID = 1196) | S5870749 | Glyma10g28250 |
| CATCCTCTTCTTTGATCCGC (SEQ ID = 1197) | GTGCTCCACTGAAAGTTGCC (SEQ ID = 1198) | CD396488 | Glyma10g34050 |
| CACCCCAAAAGTCCTTCAAA (SEQ ID = 1199) | AAGCGGATCCATGTTTATGC (SEQ ID = 1200) | BE058570 | Glyma10g41930 |
| TCAGACTTGGGTTCCTCCTC (SEQ ID = 1201) | ACCCAAACGTACCCATTTGA (SEQ ID = 1202) | S5146207 | Glyma10g42450 |
| AGATGGGTCACCATTCTTGC (SEQ ID = 1203) | CATAGCCGTGAGTGGTGATG (SEQ ID = 1204) | BE611938 | Glyma11g02400 |
| AGAAGCTCCTTGGCAAACAA (SEQ ID = 1205) | TGACATCTTGCTTCTGCTGG (SEQ ID = 1206) | BQ473403 | Glyma11g04880 |
| CCTGTTGCATACTCTTCGCA (SEQ ID = 1207) | AGGGTCATTGGAGGACGAC (SEQ ID = 1208) | S4897857 | Glyma11g05550 |
| CCAAAAGTTCTTGGGGAACA (SEQ ID = 1209) | TGGCGTGATGTTAAGCTTTG (SEQ ID = 1210) | S21538769 | Glyma11g14760 |
| TCCAAATGGGGAAATAGGTT (SEQ ID = 1211) | TGAGTGATGATGATTGGAAGG (SEQ ID = 1212) | TC209021 | Glyma11g15180 |
| ACCAAATGGAAGTTTGTCGC (SEQ ID = 1213) | CCCAGCTTCTTCCTCAGATG (SEQ ID = 1214) | S4973270 | Glyma11g33180 |
| TCAGCTCAGAATCAGCCAAA (SEQ ID = 1215) | ATCAATGCTTCCTCCATCCA (SEQ ID = 1216) | S15177336 | Glyma12g01960 |
| ATTTGTTGAGGCAGGAGCTG (SEQ ID = 1217) | AGGAAACCTGGTGCACAATC (SEQ ID = 1218) | S5126262 | Glyma12g29030 |
| TCCTTTTCTCTTCGCTTGGT (SEQ ID = 1219) | ATAACGGTGGCCTTCAGAAC (SEQ ID = 1220) | S4877491 | Glyma12g29030 |
| CTCCTGTGGTTTGCTTGTGA (SEQ ID = 1221) | TTTCTCTTGATGAAAGGGCA (SEQ ID = 1222) | TC232993 | Glyma12g36630 |
| TGTGAGGCACATTTAGGCAG (SEQ ID = 1223) | GCTTTTATGGTGATGGGGAA (SEQ ID = 1224) | TC225081 | Glyma13g05550 |
| TGGACTTGGTGAGTTTGGTG (SEQ ID = 1225) | TGTTGAATAGATCAAGGGCAGA (SEQ ID = 1226) | TC222536 | Glyma13g09980 |
| CCCATTCATATGGCCACTTC (SEQ ID = 1227) | GGGGGTGGGTTTAGGAATAA (SEQ ID = 1228) | BM092559 | Glyma13g16890 |
| TTGGATTTCCGGTACAGAGG (SEQ ID = 1229) | TTTGAAAATCCATTCCAGCC (SEQ ID = 1230) | S5141204 | Glyma13g25720 |
| ATCTCTTACGCTTTGCAGCC (SEQ ID = 1231) | GGCATCTGCAACAACTCTGA (SEQ ID = 1232) | S15850286 | Glyma13g26790 |
| TGGCTTTTTATCTTGCGTCTG (SEQ ID = 1233) | ACAAAGCAACCCAGGAAAT (SEQ ID = 1234) | S4892930 | Glyma13g38340 |
| CCCCTAGCTAGTGTGACCCA (SEQ ID = 1235) | CTCGCTATCCTATTGGATGTTT (SEQ ID = 1236) | S34273475 | Glyma13g40830 |
| GCTGTCTTCACCGGACCTTA (SEQ ID = 1237) | GCTCCAGTTGGTACTTCGGA (SEQ ID = 1238) | S21566837; | Glyma13g43120 |
| | | S34273505 | |
| TCCGGTGGTGTAATCAGCTT (SEQ ID = 1239) | TGCATGGGCTGAAACTATGA (SEQ ID = 1240) | CA785073 | Glyma14g06870 |
| TGAACTTGCAGACTTTGGGA (SEQ ID = 1241) | AAGCAATCCAAAGGGCTAGG (SEQ ID = 1242) | S5050105 | Glyma14g39130 |
| ACTTTGCGAAAAGCAAGGAA (SEQ ID = 1243) | TGACAGATTGCCTATGCTGG (SEQ ID = 1244) | S5127272 | Glyma15g03920 |
| CTGTTGAGGAACTGCCTGTG (SEQ ID = 1245) | GGCTAATTTGCTCCCTAATTG (SEQ ID = 1246) | BM955055 | Glyma15g12930 |
| TGGACCAGGAATATGCACAA (SEQ ID = 1247) | TCCCGAGACAGGATGAGAAC (SEQ ID = 1248) | S23072065 | Glyma15g14320 |
| CACCTTCCGTGAAAGAGGTAA (SEQ ID = 1249) | GCCATTAGTCTGTTTTCCATCA (SEQ ID = 1250) | BM528066 | Glyma16g01980 |
| CAAGAGAAGGAGGAAAGCCC (SEQ ID = 1251) | GGTCCTCACTGAAGAAGCCA (SEQ ID = 1252) | S34273491 | Glyma16g02570 |
| TGTTGTTGCCACCATCACTT (SEQ ID = 1253) | TGGAACACCCATCTAAGCAA (SEQ ID = 1254) | S23062212 | Glyma16g02570 |
| AAGCCAGAGACATTCCAGTG (SEQ ID = 1255) | AGTTACTGAACGGGGATTAAA (SEQ ID = 1256) | S4990094 | Glyma16g07960 |
| TTCCACTCTCCTACTTAGCCTG (SEQ ID = 1257) | TCCAAGATGATGCCATTTGA (SEQ ID = 1258) | BI469606 | Glyma16g25250 |
| CTTGCCTCTTAGGCCCTCTT (SEQ ID = 1259) | CTTGCCTTGGTTTTCCATGT (SEQ ID = 1260) | TC216457 | Glyma16g34340 |
| CCTCCAGGCAAGAGTCAATC (SEQ ID = 1261) | CGTCGTCTCTTCTTGCATTG (SEQ ID = 1262) | BE058375 | Glyma16g34490 |
| AGAGCCGGAGTAGCAGATGA (SEQ ID = 1263) | ATGGCTTCAGGGTTTGATTG (SEQ ID = 1264) | S23061916 | Glyma17g07330 |
| TCCTGTCTTTTTGGTGGGAG (SEQ ID = 1265) | CGGGGTCTGTACAAGGAACA (SEQ ID = 1266) | TC214990 | Glyma17g10250 |
| AGCATTGTTGATTGATGGGC (SEQ ID = 1267) | ATCACTGTGAATGGGCCAAA (SEQ ID = 1268) | S34273489 | Glyma17g15330 |
| TTGAACTTTGAAGTGCCGTG (SEQ ID = 1269) | TTTTGATTTCCTGTCTCACTGG (SEQ ID = 1270) | S4882412 | Glyma17g15330 |
| AAGGAGGTTTACAGCGCTCA (SEQ ID = 1271) | AATCAATCTGTTTGTGGCGG (SEQ ID = 1272) | AI938079 | Glyma17g18310 |
| AACTTGGCCTCTAATGAGGGA (SEQ ID = 1273) | CCCCTTATGGGTCCTGAAGT (SEQ ID = 1274) | CA852521 | Glyma17g36370 |
| TCCTTCCCCCTCTAGTCACA (SEQ ID = 1275) | CCAAAAGTAACTCCAATGCCA (SEQ ID = 1276) | CA936556 | Glyma18g04250 |
| CATGGCAATTTCGAGGTCTT (SEQ ID = 1277) | CTCGTAGCCGTATCAAGGAA (SEQ ID = 1278) | BG508957 | Glyma18g05900 |
| AAAATGCCTTGGCAATTCAC (SEQ ID = 1279) | CCAAGGTTTTCCCTGGTACA (SEQ ID = 1280) | CA937180 | Glyma18g18140 |
| GCACTGAGACACCTGAATCG (SEQ ID = 1281) | TTTGGGCACCAGTTTTTCTC (SEQ ID = 1282) | BE805410 | Glyma18g39740 |
| TGCAGCAAAGTTGTTGAAGG (SEQ ID = 1283) | AAGGGTTGGATGAAAAACCC (SEQ ID = 1284) | S23069986 | Glyma18g49360 |
| GGGTGGATGAAAAACACACC (SEQ ID = 1285) | AGTGCTTGTTGTGCTTCCCT (SEQ ID = 1286) | S34273430 | Glyma19g02600 |
| GCAGGGAGTGAATCAACCAT (SEQ ID = 1287) | GAGTCTTCGAAAAGGAGGGG (SEQ ID = 1288) | BU926469 | Glyma19g29670 |
| CCTTAAACGTTGCTTCCCAC (SEQ ID = 1289) | CTTGCAAATGCTGGGGTTT (SEQ ID = 1290) | S21566054 | Glyma19g30220 |
| TCATGCACCCAACATTCATC (SEQ ID = 1291) | GACACTGCACTCTCCATCCA (SEQ ID = 1292) | BU544987 | Glyma19g30220 |
| GACCCATCACGAAAAGAGGA (SEQ ID = 1293) | AAAGCTGTTTGTGCAGAGCA (SEQ ID = 1294) | S21537216 | Glyma19g40630 |
| GCCATGTAGCACATGACTCG (SEQ ID = 1295) | CCCGTTTATTCTGGGAAACA (SEQ ID = 1296) | S4993462 | Glyma20g22230 |
| TTCCCAACACAACACGTGAA (SEQ ID = 1297) | TGTTTCCCAGTTTTGAACCC (SEQ ID = 1298) | TC229776 | Glyma20g22230 |
| TGGCTTTGTTTTTCGGCTAC (SEQ ID = 1299) | TGATGAGCAGCAGCATTTTT (SEQ ID = 1300) | AW733383 | Glyma20g30250 |
| GAGGAAACATTTCTTCGGATG (SEQ ID = 1301) | CGGGTAATCGTCCTGCAATA (SEQ ID = 1302) | S5146478 | Glyma20g32510 |
| CAAAAAGCCTTGGACTGAGC (SEQ ID = 1303) | GGCAGCAGTTTGGCTATTTC (SEQ ID = 1304) | CA938036 | Glyma20g34420 |
| CCAGAGCACAAAGATGGTGA (SEQ ID = 1305) | TGGCCATGTTTTTGGATGTA (SEQ ID = 1306) | CA800552 | Glyma20g35180 |
| TCATCAATTGCAGCTTCTGAC (SEQ ID = 1307) | TGATTTTTCATCAGTCACGG (SEQ ID = 1308) | S4990921 | Glyma20g35180 |
| CAAGCTTTCAACCCCATGAT (SEQ ID = 1309) | GAAATGGGCTCAACCTGTTC (SEQ ID = 1310) | AW317542 | Glyma01g37310 |
| TTTTGGGTTCGAATTTGAGG (SEQ ID = 1311) | ACAACTATGCCTCCACCAGC (SEQ ID = 1312) | S21565729 | Glyma02g07760 |
| CACTCAGTCTCGTGCTTCCA (SEQ ID = 1313) | CCTTCTGAAATCAACACGCA (SEQ ID = 1314) | AW310386 | Glyma02g26480 |
| TTAGAATCCAATCCCTCCCC (SEQ ID = 1315) | GTTGGCACCCAAACGATAAC (SEQ ID = 1316) | BU546675 | Glyma03g30650 |
| ATCAACGGCAGAAGCAGAGT (SEQ ID = 1317) | GGATTTGGTTTTGGGGTTCT (SEQ ID = 1318) | BM271180 | Glyma05g09110 |
| CGCTGCCATCACTTTCTACA (SEQ ID = 1319) | AGAAACTGGTGCTGCCAACT (SEQ ID = 1320) | S21566467 | Glyma05g38380 |
| TCTGGGATGATGATGTTGGA (SEQ ID = 1321) | CTTTGGTGTTGTTGCCAATG (SEQ ID = 1322) | S5146166 | Glyma06g21020 |
| TTGGTTGCATCCATTGCTAA (SEQ ID = 1323) | ATGACCAATTGGGTGGTTGT (SEQ ID = 1324) | S23063408 | Glyma07g32250 |
| CATGTGTAATTCCACTGGCG (SEQ ID = 1325) | TGGGGAGGAGAGCAACTCTA (SEQ ID = 1326) | S5126778 | Glyma08g47520 |
| TTGCCAGCCTCTATCATTCC (SEQ ID = 1327) | TGATGGGTGTGAATGGAAAA (SEQ ID = 1328) | AW185294 | Glyma08g47520 |
| GATCGATTGGAAGAGCTTGG (SEQ ID = 1329) | GATCATGGTTATGGGGCATC (SEQ ID = 1330) | BE346203 | Glyma10g36050 |
| AGAATCGATACATGCGGGTT (SEQ ID = 1331) | GCAACTCACGGATCCTCGTA (SEQ ID = 1332) | S5050636 | Glyma12g35000 |
| TATTATGACTCGCATGGGCA (SEQ ID = 1333) | TGAATGGTGGAAGTGTCCAA (SEQ ID = 1334) | S21537720 | Glyma13g30800 |
| AGAAATTGAACCGGCTGATG (SEQ ID = 1335) | CCCAAAGAATCCCCACCTAT (SEQ ID = 1336) | BI892702 | Glyma13g35550 |
| CCTACAACAACGGTGCATTG (SEQ ID = 1337) | CCCTCCGTTGCTGTTACCTA (SEQ ID = 1338) | S4986242 | Glyma13g35560 |
| AAAGGTTCGAGATGCGCTTA (SEQ ID = 1339) | TGATTGATGAGCATTCAGCAG (SEQ ID = 1340) | S4981904 | Glyma13g39120 |
| ACACACAACACAGAACGACG (SEQ ID = 1341) | CTCGGGAATAATCAGATGTCG (SEQ ID = 1342) | S22952239 | Glyma14g24220 |
| TCTCCCACATGGAACACAAA (SEQ ID = 1343) | TGGAAACCAACGGGAATAGA (SEQ ID = 1344) | S5143635 | Glyma15g05690 |
| AGAAGGAAAAGTGGCACCCT (SEQ ID = 1345) | TTTGTCTCTTTGGGGACTCG (SEQ ID = 1346) | CF806665 | Glyma15g08480 |
| GCTTGGTGACCCTTTTAGGC (SEQ ID = 1347) | TGGGTTATTGCTTAGACCCTTT (SEQ ID = 1348) | BU547906 | Glyma15g40510 |
| AGCTAAGGGGCTGTCTAGGG (SEQ ID = 1349) | GATGCTGCTCAGGAAGAAGG (SEQ ID = 1350) | S5142288 | Glyma16g02200 |
| TGCTTCAGGGTATTGGAAGG (SEQ ID = 1351) | TTCACACCAACGCTCTCTTG (SEQ ID = 1352) | S4883048 | Glyma16g04740 |
| AATCAGCGGTTAATGCTTGG (SEQ ID = 1353) | TTTGGTGTGCTCAGCTTCTG (SEQ ID = 1354) | BE800180 | Glyma16g04740 |
| AAGTTGCCAATTGGGTTCAG (SEQ ID = 1355) | GTTGAGCAAACGCCTTCTTC (SEQ ID = 1356) | S6675832 | Glyma17g23740 |
| AGGACGCGTTTCGTTTTCTA (SEQ ID = 1357) | GAAGCCAGAAAGCGATCAAC (SEQ ID = 1358) | S15942527 | Glyma17g35930 |
| AACAAGACGAGAAGGAGGCA (SEQ ID = 1359) | CGTACTCTGTAATTTGGTTCAGG (SEQ ID = 1360) | CF806363 | Glyma19g40280 |
| CCGAGCTTTGAATCGAATGT (SEQ ID = 1361) | AATGGAAGTCCCTTTCTGCC (SEQ ID = 1362) | AW598682 | Glyma20g31210 |
| GCACTTCAGACATCAGGGGT (SEQ ID = 1363) | GCATAGCATGCACGTTGTTT (SEQ ID = 1364) | S4918140 | Glyma10g12530 |
| TCTTGGAGTTCCTCGTGTCA (SEQ ID = 1365) | CGACCTTTTACAATTCTTGCAG (SEQ ID = 1366) | BGT54332 | Glyma11g15530 |
| GGAAAAACCATACTTTGTCAGC (SEQ ID = 1367) | AATTTGTCCCTCCTGCATCA (SEQ ID = 1368) | TC215075 | Glyma02g12800 |
| TTTATGCCTGAGGTGACGTG (SEQ ID = 1369) | ACACATCCTCGTGCTGATTG (SEQ ID = 1370) | S5055354 | Glyma20g38260 |
| ACGCAAGGGAGAGCTGATAA (SEQ ID = 1371) | TTCCTTCCCGGACACAAGTA (SEQ ID = 1372) | AI900215 | Glyma09g06750 |
| AATCGAAGGTCTTGCTGTGG (SEQ ID = 1373) | AGTAAAGGCCCTGAACAGTTT (SEQ ID = 1374) | S23062993 | Glyma13g40460 |
| TAGCTTTGTAATGGGGCGTG (SEQ ID = 1375) | CCGTGAACTTGCACGATTAT (SEQ ID = 1376) | S4872357 | Glyma04g17600 |
| GCGATATCTCTGCTCCAAGG (SEQ ID = 1377) | ACAGTCAGGGCCAAAACAAC (SEQ ID = 1378) | S5129056 | Glyma02g41260 |
| GATGCTCAAGAAGGACGAGG (SEQ ID = 1379) | GTTGTACGCATACTGGGGCT (SEQ ID = 1380) | BU763734 | Glyma19g29260 |
| CCGGTGTTTATCCACTGCTT (SEQ ID = 1381) | GCAAGTGCATCATTTCATGG (SEQ ID = 1382) | S4918730 | Glyma06g06570 |
| AGGGGGAGAATGACGAGACT (SEQ ID = 1383) | TGCACTTTTTCCAGTTGCAC (SEQ ID = 1384) | BQ630497 | Glyma06g06570 |
| CAAGCCCATGTCCCTAAAAG (SEQ ID = 1385) | AATGGAAGCAATCAACGACC (SEQ ID = 1386) | S5126920 | Glyma08g18840 |
| TAAGCCGCCAGTGAAATCAT (SEQ ID = 1387) | GCACTTTTGGCCTGTTCAGT (SEQ ID = 1388) | S5144486 | Glyma11g01290 |
| ACATGCCAGTGAGTGCAGAT (SEQ ID = 1389) | GTGTTGGTTCAGTCCCATGT (SEQ ID = 1390) | BU926162 | Glyma09g17220 |
| CTGCAAGTACGGGGTTCACT (SEQ ID = 1391) | TTCTCCAGGGGAGATTCCTT (SEQ ID = 1392) | S22951169 | Glyma09g31080 |
| TATCAAGATGCCCCAAGAGC (SEQ ID = 1393) | GCAAAACATGGACATTGACG (SEQ ID = 1394) | BM890728 | Glyma01g39490 |
| CATGGCAATTGAAACACCTG (SEQ ID = 1395) | GTGGAAGAAATGACGGAGGA (SEQ ID = 1396) | S22952607 | Glyma01g41460 |
| TGCGATAAGCATCAAGAACG (SEQ ID = 1397) | CCGATAAGCGTGGGAAAATA (SEQ ID = 1398) | S23068862 | Glyma02g01540 |
| GAGTGGGCAAATCCCAAATA (SEQ ID = 1399) | TGCTTGGGCTCCTCATAGTT (SEQ ID = 1400) | S15924495 | Glyma04g40610 |
| GGCAGAAACAGTTGCCTCAT (SEQ ID = 1401) | AGCAACAATAGATCCGTGGG (SEQ ID = 1402) | BE330878 | Glyma10g01580 |
| GTTCTTCCGTGTTTTCGGAC (SEQ ID = 1403) | CTTGGCTGCCACATACAGAA (SEQ ID = 1404) | CA785184 | Glyma10g31970 |
| TGGGGGAATCCATGTTATTG (SEQ ID = 1405) | ACACCTTGTTGATTGCGTTG (SEQ ID = 1406) | BI426372 | Glyma14g13790 |
| CCACCTTGAGTTAACACCTCG (SEQ ID = 1407) | GCATTATGGTGCTGTTCCCT (SEQ ID = 1408) | BU544012 | Glyma17g10770 |
| ATTAATTCGCTTCGTGGTGC (SEQ ID = 1409) | CCAAAGTGCCGAGGTATTGT (SEQ ID = 1410) | S21538807 | Glyma18g51890 |
| TCCAAGCTGTATCTGGCCTT (SEQ ID = 1411) | CCGTGGTTCTTTTGGTTGAT (SEQ ID = 1412) | BU545160 | Glyma13g25640 |
| AGTCCACCCACAGGTTTCAC (SEQ ID = 1413) | ATGCCTTTACATTCGCATCC (SEQ ID = 1414) | S4977219 | Glyma19g27690 |
| GGCAAATTCAATTCTTGGGA (SEQ ID = 1415) | TAAAACTGAGGGGCCTGATG (SEQ ID = 1416) | S21700413 | Glyma01g02210 |
| CTCAAGCCACTTCATTTGGT (SEQ ID = 1417) | TTTCCCAAGAAACTACCTTCC (SEQ ID = 1418) | S5045510 | Glyma01g04610 |
| AGAATTCATCCCCTCCTTGA (SEQ ID = 1419) | TGATGATGATGATGATATGCAC (SEQ ID = 1420) | S15852371 | Glyma01g23010 |
| GTGCAGGATGTCTACGGGAC (SEQ ID = 1421) | GGCTTTCTCAGCTTTGGGTA (SEQ ID = 1422) | S4916603 | Glyma01g23010 |
| TGGTTCATGGCTTTGTGAGA (SEQ ID = 1423) | TGACCCAAACGGAGAAGAAG (SEQ ID = 1424) | S4983140 | Glyma01g24880 |
| CACCTTGCAGAATATCCGGT (SEQ ID = 1425) | CAAAAGCTTGGGAAACCAAA (SEQ ID = 1426) | S4989469 | Glyma01g44670 |
| AAAGTGGCGGTTGTTGAAAG (SEQ ID = 1427) | AAAGGTGGAGCAATGCAATC (SEQ ID = 1428) | CA783023 | Glyma02g01680 |
| AGCAATGGTGGAGCCATAAG (SEQ ID = 1429) | CCGGACAGTCTTCCCAGTAG (SEQ ID = 1430) | S21538340 | Glyma02g01760 |
| TGGAGTGACGACGATGAGTC (SEQ ID = 1431) | ATGCTTTGGAGTTTTCCCCT (SEQ ID = 1432) | S5026438 | Glyma02g16410 |
| CCAGCGCTGATTTGATGTTA (SEQ ID = 1433) | CCAGCAGAAAGCTCCAAAAC (SEQ ID = 1434) | S4869132 | Glyma02g17160 |
| CTCTCACCCAAAATCCCTCA (SEQ ID = 1435) | ATGGCTAATGGATCCCCTTT (SEQ ID = 1436) | S5035276 | Glyma02g18680 |
| GATGACAAGGTCCCACGAAT (SEQ ID = 1437) | GCCAAGCAACCTCTTCTTTG (SEQ ID = 1438) | BU550564 | Glyma02g44040 |
| GGAGAAGTGAGGTGTGAGGC (SEQ ID = 1439) | AATTTGTGGGCTCCACTGTC (SEQ ID = 1440) | BM094448 | Glyma02g48040 |
| GTTCAGTGTTGCAGCCATGT (SEQ ID = 1441) | AACCTACCCAACGTAGCAAAA (SEQ ID = 1442) | S5130128 | Glyma04g39480 |
| TGAAGATCCCCAATCCCATA (SEQ ID = 1443) | CTTTGGTGGCTCGGATCTAA (SEQ ID = 1444) | S19679391 | Glyma05g11200 |
| ATCTGGCTTTGCCAATTTGT (SEQ ID = 1445) | GTCAGGCATTTCCTGCTTCT (SEQ ID = 1446) | BU548721 | Glyma05g11200 |
| TTATCCGAGTCCATTTTGGG (SEQ ID = 1447) | GCCATTCAGAACACGAGGTT (SEQ ID = 1448) | S17641808 | Glyma05g13530 |
| TAGGCCCTTTCAACCACAAC (SEQ ID = 1449) | ATCCAGCTGTCCGAACTTGT (SEQ ID = 1450) | BE346622 | Glyma05g25630 |
| GAGAACCAAACGCTGGATGT (SEQ ID = 1451) | GCGAGTCCTTTTCACCACTC (SEQ ID = 1452) | S4918062 | Glyma05g29300 |
| ACATTATGGCTTGTGCCGAT (SEQ ID = 1453) | ACTGTGTCATGATTCGCAGC (SEQ ID = 1454) | S4868859 | Glyma05g34980 |
| AGACCAAGACCAGAACGACG (SEQ ID = 1455) | GCTCCAAACAAAGAAACCCA (SEQ ID = 1456) | S21537813 | Glyma06g01300 |
| CTGCAGGGTAGAGTTGGAGC (SEQ ID = 1457) | GTGCATCTTCATCAACACCG (SEQ ID = 1458) | S21537673 | Glyma06g08790 |
| AGGAACCCCCTGAGAGCTAC (SEQ ID = 1459) | GCAAAGAAGAACGACAGAGGA (SEQ ID = 1460) | S16521981 | Glyma06g15490 |
| ACGCCTATGAACGTGAAACC (SEQ ID = 1461) | GCATTCGGTGGGAATTAGAA (SEQ ID = 1462) | S17640718 | Glyma06g26610 |
| GGGAAAACCTCATGAGTCCA (SEQ ID = 1463) | GTCCGGTAGGCTCGATACAA (SEQ ID = 1464) | BE658021 | Glyma07g04780 |
| GGAGTTGTTGTGAGCGTGTG (SEQ ID = 1465) | TATTTGATCGTAGATCCAGCAC (SEQ ID = 1466) | S5023085 | Glyma07g16420 |
| TGGTTTGTGCAAATATCCCC (SEQ ID = 1467) | CAATTGTGAGAAAGAGCGCA (SEQ ID = 1468) | S4891180 | Glyma07g28520 |
| AGAAGTTGTGCAAAATGGGG (SEQ ID = 1469) | TTGTGCAAGATCCCCTAACC (SEQ ID = 1470) | S4925169 | Glyma07g30140 |
| GAGAGAGGGAAGCCCGTTAG (SEQ ID = 1471) | TCCACCAATAACACCAACCA (SEQ ID = 1472) | S5030137 | Glyma07g32770 |
| TTTAGGACAGTTGCTTGGGC (SEQ ID = 1473) | GAGAGTGTCGGGGATGTGTT (SEQ ID = 1474) | S5088770 | Glyma07g37000 |
| CCCATGGAGCAAATACACCT (SEQ ID = 1475) | AGCAAGCAAAAGTTTCCAGG (SEQ ID = 1476) | S21567824 | Glyma08g04760 |
| GTCCGATTGGAGAATCATGC (SEQ ID = 1477) | GAATCTCAAATTCGGTCCCA (SEQ ID = 1478) | S4903121 | Glyma08g07170 |
| TATGGGGCTATACCGCTACG (SEQ ID = 1479) | CGCCTTCTATACCCACTGGA (SEQ ID = 1480) | S4866857 | Glyma08g12460 |
| CTCTTCACGGACTTCTTGCC (SEQ ID = 1481) | AAGGATCGCGTTTAGAACCA (SEQ ID = 1482) | S23065233 | Glyma08g15050 |
| CGCGTCCGATAACAATAACA (SEQ ID = 1483) | AGAGAATTGCCGATGGTGAT (SEQ ID = 1484) | S18956636 | Glyma08g16370 |
| CCCAGATGCTTACACAAAAGC (SEQ ID = 1485) | CAGAATTTGAGTGCGCTTGA (SEQ ID = 1486) | S4911119 | Glyma08g16830 |
| AGGCAAAAGGGGATAAATGC (SEQ ID = 1487) | GCTTGTTTCAAATGGCTCGT (SEQ ID = 1488) | BQ453457 | Glyma08g23240 |
| AGGCACTTTGTTTTCCCTTG (SEQ ID = 1489) | TGCATGTTTACTGCAGCGAT (SEQ ID = 1490) | S5101279 | Glyma08g47570 |
| AAACTGGAGCTTTGACACCAA (SEQ ID = 1491) | ATATGTTCATCCCTGGCTGC (SEQ ID = 1492) | S4973725 | Glyma09g06690 |
| AAAGAAGCCAACAGGCAGAA (SEQ ID = 1493) | CCTTCCGATGCAGAAATCAT (SEQ ID = 1494) | S4925834 | Glyma09g11870 |
| AAGTTGTATGGTTGGGCCTG (SEQ ID = 1495) | ATCCCCGCCTCATACTATCC (SEQ ID = 1496) | S21565790 | Glyma09g18050 |
| TTGATGTGGAAAGGGGACAC (SEQ ID = 1497) | CGTTGGCAAAGTTATCGGTT (SEQ ID = 1498) | S4903128 | Glyma10g02890 |
| GTGTGTTGAGGGGTTTTGGT (SEQ ID = 1499) | CTCTGCTTCTGCTTGAACCC (SEQ ID = 1500) | BM522547 | Glyma10g21570 |
| ATGTGGTTGTTGTTGGTTGG (SEQ ID = 1501) | CACTTGACAGCTGAATTCCAGTA (SEQ ID = 1502) | S5100930 | Glyma10g37390 |
| GGCCGTGTTAAAACGTGTG (SEQ ID = 1503) | GGCTTTTGCTTTAGCCAGTG (SEQ ID = 1504) | S4883701 | Glyma10g42460 |
| GTTTACGCAAACACCGACCT (SEQ ID = 1505) | ATTGGATGCAGAGGGTTTTG (SEQ ID = 1506) | BM085598 | Glyma10g42900 |
| CGACAAGAAGAATGCGAACA (SEQ ID = 1507) | CTGAGACTCACTGGCCTTCC (SEQ ID = 1508) | BQ630507 | Glyma11g08110 |
| CCAAGATCAAGTGCAACACC (SEQ ID = 1509) | GGACCCATGTGAAATTGACC (SEQ ID = 1510) | S5011331 | Glyma11g08590 |
| GCACTGTTTTTCCATCGTCA (SEQ ID = 1511) | CTCGTGACCATTGTGGTTTG (SEQ ID = 1512) | S21539044 | Glyma11g10910 |
| TGCTGGGTGATATTGGTGAA (SEQ ID = 1513) | GTCTCTGCTGGCACCATTCT (SEQ ID = 1514) | S4934473 | Glyma11g12560 |
| ATGGGGAGCATATGCAGTGT (SEQ ID = 1515) | TCGACCAAGTAGGGTCTTGA (SEQ ID = 1516) | BE820313 | Glyma11g20080 |
| CAAGGCTGTTCCAACACAAA (SEQ ID = 1517) | TAGCCATCATCAAGACGCAG (SEQ ID = 1518) | S21566925 | Glyma12g03130 |
| ATGGCCAATTGGAGTATTGC (SEQ ID = 1519) | GGACAACCAGTCAAGGGAAA (SEQ ID = 1520) | S21539619 | Glyma12g14030 |
| CGTCGGATTAGAACCCTTGA (SEQ ID = 1521) | GCTTTTTCACGAAAGCAACC (SEQ ID = 1522) | TC229886 | Glyma13g01310 |
| ATCACAATGCTTGGAGACCC (SEQ ID = 1523) | TGTGCTTGTCTGAGTCCTGG (SEQ ID = 1524) | S4911726 | Glyma13g31720 |
| 1TTTTCCTCGCAGTTATGCC (SEQ ID = 1525) | TCCAAAGACTAAGAGGGGGAA (SEQ ID = 1526) | S4954000 | Glyma13g37320 |
| TGCCATGCGTATTTTCTGAG (SEQ ID = 1527) | GGCCGCAAGCTTTTTAATCT (SEQ ID = 1528) | S4937572 | Glyma13g39990 |
| ACAAGCGAAGGAAGGAGTGA (SEQ ID = 1529) | GTCCGTCCCTTGCTATTCAA (SEQ ID = 1530) | S5035841 | Glyma14g00670 |
| GTCCCTTTGCAGTGGTGACT (SEQ ID = 1531) | TCAAGATCTGCCACCAAATG (SEQ ID = 1532) | S15925681 | Glyma14g03340 |
| CTCTGCTGGTGGAAGTTGGT (SEQ ID = 1533) | GATCCCGAAATCATCCGTAA (SEQ ID = 1534) | S4876235 | Glyma15g03810 |
| TATTTAAAGGTGGTCGCCCT (SEQ ID = 1535) | ATGACAGCGATGAAGAGGCT (SEQ ID = 1536) | S23064226 | Glyma15g36170 |
| ACTGCATTCATTCCGGTTTC (SEQ ID = 1537) | GGAAGAAATCCTTCGGGTTC (SEQ ID = 1538) | BU761035 | Glyma15g37270 |
| TTTTGGACGGCTAAGTGTCA (SEQ ID = 1539) | TCAGATAAGGTGCGCAGTTG (SEQ ID = 1540) | S21566203 | Glyma17g13090 |
| GGATTCAGTCACAGCAGCAA (SEQ ID = 1541) | ACACCGAGAGACGACCAGAC (SEQ ID = 1542) | S4936226 | Glyma17g15240 |
| CAGTGGGAGAAGGAGCGATA (SEQ ID = 1543) | CCGAAATATCGGAAGGGATT (SEQ ID = 1544) | TC216262 | Glyma17g33500 |
| GCCTCTTGATGACACTGCAA (SEQ ID = 1545) | TTCAATGCACTCTCCACTGC (SEQ ID = 1546) | S18530324 | Glyma17g35230 |
| TTTTCGAACAGCCTCCCTAA (SEQ ID = 1547) | ATGCGGAGTGATGGTTATGT (SEQ ID = 1548) | S21540325 | Glyma17g37310 |
| CATCTACGGGTACTGGCGAT (SEQ ID = 1549) | TCCGGAAACCAGAACTTGAC (SEQ ID = 1550) | S4992048 | Glyma18g01040 |
| TGCTTGAGCAAGGTTTTGTG (SEQ ID = 1551) | AACATGGCTGACGTATGGGT (SEQ ID = 1552) | CD412532 | Glyma18g03990 |
| GCAACTCGTGAAAGGTAGGC (SEQ ID = 1553) | TTTCATCCGGCACAGTATCA (SEQ ID = 1554) | CD399559 | Glyma18g08720 |
| TCCATTGAGGAATTGCATGA (SEQ ID = 1555) | GCGTTGAAACAGATTTGGGT (SEQ ID = 1556) | TC231646 | Glyma18g47300 |
| CGTTCATCAATGGCAGAAGA (SEQ ID = 1557) | AAGGAGCATTGCTGCATTTT (SEQ D = 1558) | S21537328 | Glyma18g48000 |
| CCATGGATGCTGAGGAACTT (SEQ ID = 1559) | CTGCCACTTCATCCTTTGGT (SEQ ID = 1560) | TC220047 | Glyma19g36270 |
| ACAATCAACCGAGGCTCAAC (SEQ ID = 1561) | CGAATCATCGTCCTCATCCT (SEQ ID = 1562) | S5146199 | Glyma19g37410 |
| CCCAGGTATGGTCCTTCTCA (SEQ ID = 1563) | CTTCTACCCCATGGCAAGAG (SEQ ID = 1564) | CD395499 | Glyma20g38050 |
| CCGTGCTGTTGTGGAATATG (SEQ ID = 1565) | ACCAGGACACCTGACTCCAG (SEQ ID = 1566) | BG238414 | Glyma04g38010 |
| CCGGTCTTTCTAGGAGGAGG (SEQ ID = 1567) | TCCAGGATGAAGCAAAGACC (SEQ ID = 1568) | BU544268 | Glyma06g17050 |
| GGCCGTAGTTGACTGTAGGG (SEQ ID = 1569) | AGTTGAATCCCCCAACGACT (SEQ ID = 1570) | S21540167 | Glyma06g17050 |
| GTGTCCAAAAATGGGCAATC (SEQ ID = 1571) | TGACGACCAATGAGGTGTGT (SEQ ID = 1572) | AW568684 | Glyma06g17050 |
| CACAAAAACCTCAACTGCGA (SEQ ID = 1573) | AATAAAAGGTGCATGTGGCA (SEQ ID = 1574) | S23063598 | Glyma08g00910 |
| TGCATTTTACCCCCTTTGAA (SEQ ID = 1575) | AGGGTTTTGGGGATTTTGTC (SEQ ID = 1576) | S4911429 | Glyma10g02980 |
| CGGAAACCCTACGGTAGACA (SEQ ID = 1577) | CAGTGCTTCGGGAAGATAGG (SEQ ID = 1578) | AW831041 | Glyma01g03570 |
| GGTTGACTATTTCCACCTACCT (SEQ ID = 1579) | TGCTGTCTTTTTGTCTCAGTG (SEQ ID = 1580) | S4994979 | Glyma07g31650 |
| AAAAAGACGACCACAGCGAC (SEQ ID = 1581) | ATCATCGTCGTCGTCATCAA (SEQ ID = 1582) | AW153030 | Glyma13g24790 |
| CATCAATTCAAGAGAATGGGG (SEQ ID = 1583) | CTTCTGAAGAATGCCTAATTGC (SEQ ID = 1584) | BU549127 | Glyma15g41230 |
| AGCAGCAGGACAGAACAGGT (SEQ ID = 1585) | AGCAGCCCTACATGGACATC (SEQ ID = 1586) | S21539760 | Glyma06g07110 |
| CGAAAGGATGAAACTCTCGC (SEQ ID = 1587) | GCCAAATACTTTCCGATCCA (SEQ ID = 1588) | S4891446 | Glyma13g40460 |
| CGAAACGGAACCAAAGAAGA (SEQ ID = 1589) | CTTCAACCTCGGGTGATTGT (SEQ ID = 1590) | BQ613064 | Glyma13g41500 |
| GAGGAATCGACGTTGGTGAT (SEQ ID = 1591) | CCGTCTCTTTCCATCTGCTC (SEQ ID = 1592) | S4933793 | Glyma17g09900 |
| TACCCTTTCCCTGCTCCTCT (SEQ ID = 1593) | CGATTGACAACTCAACCGAG (SEQ ID = 1594) | S4991114 | Glyma02g09030 |
| TGATGGTATTGCTGCTCCAG (SEQ ID = 1595) | TGCTGCAGATCCTGTTTTTG (SEQ ID = 1596) | CF808484 | Glyma01g00980 |
| TCAAAATTGTTGGCCAGTGA (SEQ ID = 1597) | TCTTGTGCTTGTTTCATCGC (SEQ ID = 1598) | S15933266 | Glyma09g15750 |
| TGCTCATTGCTACCTCAACG (SEQ ID = 1599) | ACGGCCATAGATCACCAAAG (SEQ ID = 1600) | S23068376 | Glyma0022s00470 |
| TTCGGAACAGTTTGTCGAAG (SEQ ID = 1601) | GACCAATCACAACACATGCC (SEQ ID = 1602) | BG362762 | Glyma11g08610 |
| ATATGATGACTGCCACGGGT (SEQ ID = 1603) | TGCTGTCCTCTCGAATGATG (SEQ ID = 1604) | S18957274 | Glyma11g15530 |
| CCACCTTCCCCATGATACAC (SEQ ID = 1605) | AGAAGACATGCCCTGGACTG (SEQ ID = 1606) | S21565951 | Glyma15g18790 |
| TACCTATCACCGAGAAGCGG (SEQ ID = 1607) | ATATGTTCCTGGCGAAAACG (SEQ ID = 1608) | S15926407 | Glyma20g34690 |
| GTGAGGGAGAGACGAAGACG (SEQ ID = 1609) | CTCCATTCCCTCTCACGAAA (SEQ ID = 1610) | S23071286 | Glyma03g28510 |
| TCAAGGGCATGGCTATAGGT (SEQ ID = 1611) | CCAGCACGGTTGGATTATCT (SEQ ID = 1612) | S23067653 | Glyma14g31370 |
| ATGAAGCTGCAGCCAAACTT (SEQ ID = 1613) | CTTCCTCCTCCTCCACAAGA (SEQ ID = 1614) | S5057766 | Glyma14g31370 |
| ACCATCGTCCGTTCATCAAT (SEQ ID = 1615) | TCCTCAGGGAGTTGTTTTGG (SEQ ID = 1616) | S4989926 | Glyma20g36110 |
| GTTGTGCCAGCATTTCTTGA (SEQ ID = 1617) | AATTTGAGCCCACAGGTCAG (SEQ ID = 1618) | AW201880 | Glyma20g36110 |
| ATTCGGCACGAGGGTAATC (SEQ ID = 1619) | CAACATCGTAAGGAACATTAGGC (SEQ ID = 1620) | BG653915 | Glyma03g37950 |
| ACAGCCAGAGCCTCGTTAAA (SEQ ID = 1621) | ACGAAGAGGCAGCTGAAGTC (SEQ ID = 1622) | S21537528 | Glyma01g01210 |
| TTACAAGCTGTGGATGTGCC (SEQ ID = 1623) | TGGATGAGGTCTTGGTCCTT (SEQ ID = 1624) | BI321021 | Glyma02g09470 |
| CAAATTGGGGTTTCCTTCG (SEQ ID = 1625) | TTTGCTTGTCGAGTTCGATG (SEQ ID = 1626) | S5025673 | Glyma01g08060 |
| GTGATGAGCGAACTGTGCAT (SEQ ID = 1627) | TGCCAGATAAGGCTGCAGTA (SEQ ID = 1628) | S4876508 | Glyma02g01160 |
| GAGCTCAGTCTTCCTCGTCG (SEQ ID = 1629) | AGGGTTCGTGCTTTGGTATG (SEQ ID = 1630) | S6675747 | Glyma03g27180 |
| AGCGGGTAGAGTTCACGTTG (SEQ ID = 1631) | TATTGTTGACGCTCCTCCGT (SEQ ID = 1632) | BG650304 | Glyma07g14610 |
| TATGGTGGCATGAAAACAGC (SEQ ID = 1633) | TGAGCTTTTGAAGAGCAAAGC (SEQ ID = 1634) | S5117294 | Glyma07g36180 |
| ATATGCACCCCCAGACAAAA (SEQ ID = 1635) | AAGGCCACTGGAATCATCAG (SEQ ID = 1636) | BU578952 | Glyma11g36980 |
| GCACGTGTTGTTGGTTTTTG (SEQ ID = 1637) | TATGACTATGCATCCCTGCG (SEQ ID = 1638) | S23070894 | Glyma15g21860 |
| CCCCAATGTAACTTTCCCCT (SEQ ID = 1639) | CACACTTAGCTGGAATGGCA (SEQ ID = 1640) | S23068686 | Glyma19g32800 |
| GATTGGGTTGAAGTGTTGGG (SEQ ID = 1641) | GCAAGTTTATGGGCAACCAG (SEQ ID = 1642) | BM092903 | Glyma20g00900 |
| CATTGGTTCATATCCCCCAC (SEQ ID = 1643) | CCTAGCCGCTACTCTCCCTT (SEQ ID = 1644) | BU551328 | Glyma01g33260 |
| GAATCCGACATAGGCCAGAA (SEQ ID = 1645) | ACCCCAGATTCCAACCTCTC (SEQ ID = 1646) | BE473856 | Glyma13g38080 |
| CCATTCCCATGGAAAACAAC (SEQ ID = 1647) | GGCATTTGGCTAGGATTGAA (SEQ ID = 1648) | S23064758 | Glyma02g12280 |
| GTGGTCTCAGCCTTCAGGAC (SEQ ID = 1649) | TAAGTACAAAACCGGCACCC (SEQ ID = 1650) | AW759718 | Glyma03g33970 |
| CTGAACAGCGGTACCAGGAT (SEQ ID = 1651) | GCAGCCAGGTTCTCTGATTT (SEQ ID = 1652) | S5101165 | Glyma10g06500 |
| CTGCAGACTCAGCAATTGAGAT (SEQ ID = 1653) | AGCCTGATTATGCCCCTTTC (SEQ ID = 1654) | BQ272709 | Glyma19g36710 |
| CGTGCATTTATTTTCAGGGG (SEQ ID = 1655) | ATGAGGCTGGTGCTGCTACT (SEQ ID = 1656) | S4991641 | Glyma04g38730 |
| CTGGTACATACAACGTGCCG (SEQ ID = 1657) | ACTCGGAGGATCTGCTTCTG (SEQ ID = 1658) | S4965728 | Glyma04g38730 |
| GATGGAAGAGAACGAGCGAC (SEQ ID = 1659) | CCGAAGACTGACCTTCATCC (SEQ ID = 1660) | S5109674; | Glyma01g02880 |
| | | BQ610438 | |
| AGTCTGCAAGGAAGAAGGCA (SEQ ID = 1661) | TTGGGCTGATAGCGTCTTTT (SEQ ID = 1662) | BU927363 | Glyma01g13950 |
| TCATTCGTTCATCAGTGGGA (SEQ ID = 1663) | TTCATCACTTTCTGGCGTTG (SEQ ID = 1664) | S5015932 | Glyma02g38370 |
| CGATTGCAAGGAAGAGGAAG (SEQ ID = 1665) | CTATTGCATTTCTCGACGCA (SEQ ID = 1666) | S4916150 | Glyma03g33900 |
| AGCAGAGGCAACAGTATCCAA (SEQ ID = 1667) | CTGCTGTCAATGGCACAGAT (SEQ ID = 1668) | S5128683 | Glyma04g01600 |
| TCTTCTGGAAGCTATTTCGCA (SEQ ID = 1669) | ATTGATTCGCAAAAGGAAGC (SEQ ID = 1670) | BQ296202 | Glyma04g01600 |
| GGTCCGCAGAGGATTTTGTA (SEQ ID = 1671) | CCCATGCTTCAAAGCAGATT (SEQ ID = 1672) | S5020524 | Glyma04g42200 |
| AGCCTGACATAAGGTGTGCC (SEQ ID = 1673) | GACATGTATTCTCCCGGTGG (SEQ ID = 1674) | BU550308 | Glyma06g21530 |
| GGGAAGTGCAATAATGAAGCA (SEQ ID = 1675) | TACGTAGAAGAAAGGGCCGA (SEQ ID = 1676) | BU761371 | Glyma11g07220 |
| GGTGGCTCTTCTGATGCTCT (SEQ ID = 1677) | GGTCGAGATACAAAGCCTGC (SEQ ID = 1678) | S4980774 | Glyma12g31910 |
| CTCAGCCATGCAATTCTTCA (SEQ ID = 1679) | ATTGTTTTGGGAAGCACAGC (SEQ ID = 1680) | S4915127 | Glyma15g07590 |
| GCATACAACAAGTTCACCCG (SEQ ID = 1681) | AAGTCCATTTGCCACAGAGG (SEQ ID = 1682) | S15847407 | Glyma16g03950 |
| ATTGTTGAGGCCTGTATCGG (SEQ ID = 1683) | TGATGGCAGCTTTTAGGTCC (SEQ ID = 1684) | S4980388 | Glyma04g42590 |
| GAAGCCGGTGTCAAGGACTA (SEQ ID = 1685) | GGACACTACTCTCGGCTGCT (SEQ ID = 1686) | S5030305 | Glyma14g24290 |
| GGCTGAGCTAACTTTGAGCG (SEQ ID = 1687) | TGAAGTCCTGAATCAGTAGCCA (SEQ ID = 1688) | CA938591 | Glyma02g10220 |
| AAACCATTCACTGTTTGCTGG (SEQ ID = 1689) | TGGTTAACCGAAGGGTTTCA (SEQ ID = 1690) | S4916506 | Glyma05g07750 |
| TTCCCAGCCAAATTTAAGGA (SEQ ID = 1691) | GGAATATGCAAGACCCTCCA (SEQ ID = 1692) | S5146784 | Glyma16g25450 |
| ACATATGGATGGTGGCCAAT (SEQ ID = 1693) | TGCCTCGATACAAAGCACTG (SEQ ID = 1694) | S5032746 | Glyma05g01130 |
| TTTGAACCAAGCCAAAAACC (SEQ ID = 1695) | GTGGACCTAACAATGTGCCC (SEQ ID = 1696) | BQ297035 | Glyma06g43720 |
| GCTGGTGATGGTTGTTGTTG (SEQ ID = 1697) | TCGCCTATAGACGGATCCAC (SEQ ID = 1698) | S21567689 | Glyma08g10350 |
| AAGGTTGAAAAGCTGCGAAA (SEQ ID = 1699) | GCACTGCATCTACACCCAAA (SEQ ID = 1700) | S4877244 | Glyma08g12970 |
| TGAGAAGTTCCGAAGATCGAA (SEQ ID = 1701) | GTTGAAGAGCATAGGGGCAA (SEQ ID = 1702) | S21537611 | Glyma10g42280 |
| CTGCTTCCTCCGATTCTCAC (SEQ ID = 1703) | CCCAATTGATTCCAAGGAGA (SEQ ID = 1704) | BG044834 | Glyma12g35720 |
| CTCCAGAACCAGTAGCCAGG (SEQ ID = 1705) | GCTCGTTGTTGTTGTGGTTG (SEQ ID = 1706) | BE804085 | Glyma13g34690 |
| CCCCATATTGTTCTTTCTCCC (SEQ ID = 1707) | TTAAGGGCAGACCAAAGCAG (SEQ ID = 1708) | S4875309 | Glyma16g05840 |
| ACCAGCCTTTCCCAACTTTT (SEQ ID = 1709) | TCAGATGGGTTGGTGGTGTA (SEQ ID = 1710) | S23071068 | Glyma18g01580 |
| TGCTGGCTGAGGTTTCTACA (SEQ ID = 1711) | AAGGGGCTAAACCAAATCCA (SEQ ID = 1712) | TC205922 | Glyma19g26560 |
| TGCTGTTGGGTGAATGAAGA (SEQ ID = 1713) | GTTCTCAAAATCCATTGGCG (SEQ ID = 1714) | S5002246 | Glyma19g29330 |
| GTCGGACTTGTGTCCCAGTT (SEQ ID = 1715) | ACACGAAAGGTGGAGGGTC (SEQ ID = 1716) | S23071353 | Glyma20g29330 |
| GAGGTTGGCCTCCATTGATA (SEQ ID = 1717) | TCTCTCTCTTGGTGTTGGGC (SEQ ID = 1718) | TC210810 | Glyma08g05240 |
| TGACCGGGTTTCAGGAGTAA (SEQ ID = 1719) | TCTCCATCCATCCCTTTCTG (SEQ ID = 1720) | S4925034 | Glyma11g34050 |
| CGGCACTGGTTTCCAAGATA (SEQ ID = 1721) | TCAGCAACGTTCGTCATTTC (SEQ ID = 1722) | S4897670 | Glyma11g14450 |
| TCGACCTCTCCAAATCTGCT (SEQ ID = 1723) | TTGTAAGTGGAAGGGGCATC (SEQ ID = 1724) | S21539162 | Glyma13g41390 |
| ACAGCATCAACCTTAGCCGT (SEQ ID = 1725) | TTACACCCCAGCTGTTCCTC (SEQ ID = 1726) | S21540786 | Glyma01g38090 |
| ATGTGCCCAATTCTGCTACC (SEQ ID = 1727) | AGTTGCTAGTTCCGGCAAGA (SEQ ID = 1728) | S4898759 | Glyma02g38030 |
| GACCAATCATTCCAGGCATT (SEQ ID = 1729) | GCCGAGAGAGGACAAACAAA (SEQ ID = 1730) | S23070876 | Glyma06g03070 |
| TGTTGCTTGTCTTGCTTTGC (SEQ ID = 1731) | AAGTGCGGTTTTCAATGTCC (SEQ ID = 1732) | S23063028 | Glyma05g24700 |
| TTCTGCCCTTTCTGATTTCC (SEQ ID = 1733) | GCCAAGTAATGCTCCACCAA (SEQ ID = 1734) | TC227176 | GTyma18g06110 |
| GCCATTTCTCTTAGGGGGTT (SEQ ID = 1735) | GGGAAAGGGGTTTCACAGA (SEQ ID = 1736) | S4866988 | Glyma17g00250 |
| AAGACCCTGCGGGCTACTAT (SEQ ID = 1737) | AAGCTGAACCAAGTGCCTGT (SEQ ID = 1738) | S23069945 | Glyma13g11200 |
| GCAAATTCATGGAAGAGGGA (SEQ ID = 1739) | AATTGCTTCCTGGACCGTAA (SEQ ID = 1740) | S4872880 | Glyma04g03310 |
| GATCACTCAGAATCCAGGGC (SEQ ID = 1741) | GCATCGCATCAGTACAACCA (SEQ ID = 1742) | S22952242 | Glyma07g21160 |
| CATTGCAAAGCAAGGGTTTT (SEQ ID = 1743) | ACGCGATTGAGTTTTGATCC (SEQ ID = 1744) | BE802348 | Glyma07g21160 |
| TGAGTCGATATGTTTGTGCCA (SEQ ID = 1745) | CCCCCTCGAGGTATTTTATGA (SEQ ID = 1746) | S4912396 | Glyma07g21160 |
| TCACGCCATGTGCTCTACTC (SEQ ID = 1747) | AGGAGAGAGACGCCACAGAA (SEQ ID = 1748) | S4865868 | Glyma12g04380 |
| TGTTACTTCTGGTGGTCCCC (SEQ ID = 1749) | CCAGACAGCGCAATGAAATA (SEQ ID = 1750) | S4907392 | Glyma12g33130 |
| ATGAATTTGGTCCTTTCGCT (SEQ ID = 1751) | GTCATGCACCTGCTTCATATT (SEQ ID = 1752) | TC230059 | Glyma17g10130 |
| CGGACGTCAAGAACACAAGA (SEQ ID = 1753) | ATTAGGCGTATTGGTGACCG (SEQ ID = 1754) | S4981395 | Glyma11g09750 |
| CTGCAAAGTTGTTGCTTGGA (SEQ ID = 1755) | TGGAGGATAACACATTCGCA (SEQ ID = 1756) | S4885448 | Glyma06g19840 |
| CAATAAATGCACGCAACCTG (SEQ ID = 1757) | CTGCACGGTCAAAGCATCTA (SEQ ID = 1758) | S23071155 | Glyma17g10130 |
| CCAGATCGAATCAATGGAAAG (SEQ ID = 1759) | TACCAGGCTGCAATGCATAA (SEQ ID = 1760) | S4904547 | Glyma11g34010 |
| CAAGCTTTTACACCAGAGCAGA (SEQ ID = 1761) | TCGTTGCCCATCATAGTTCA (SEQ ID = 1762) | BI785471 | Glyma05g38060 |
| GTTCCTTCTTTGGAGTTGCG (SEQ ID = 1763) | CTTCAAAGCCAACAGCAACA (SEQ ID = 1764) | S22952966 | Glyma09g01260 |
| ATTCTTCCATGATGGGGGTT (SEQ ID = 1765) | CCTGAGCAAGAGTGGAGGAC (SEQ ID = 1766) | BM521609 | Glyma18g10040 |
| TACCACTCTCCACCTCCACC (SEQ ID = 1767) | CCATGTTGTGGATTCAGTGC (SEQ ID = 1768) | BE330208 | Glyma03g00420 |
| TTAAGTCTGAAACTGGAAGTGC (SEQ ID = 1769) | CCTCTCCACGTTGTTCCTTT (SEQ ID = 1770) | AW308923 | Glyma06g23400 |
| CCTTGTTTGTGTGTTCAGGC (SEQ ID = 1771) | CTTTGGCAGATTCGAGGAAG (SEQ ID = 1772) | BG155054 | Glyma05g24700 |
| TCAACCAAGGACAATTAGCA (SEQ ID = 1773) | GCACATCGTGACTAGCAGGT (SEQ ID = 1774) | CD395607 | Glyma19g28580 |
| GCGACATCTTGGTTCTTATTTG (SEQ ID = 1775) | AAGGCATTTTTCCTTCTCTGG (SEQ ID = 1776) | S22952516 | Glyma02g07830 |
| CTGCTGCAGTTGGTAACCG (SEQ ID = 1777) | ATTCCCTCCTCCAACCATGT (SEQ ID = 1778) | BU761888 | Glyma11g15480 |
| TTCTTTTGTCGTCTCGGACC (SEQ ID = 1779) | CCCTAAATCGGAACCAGAAA (SEQ ID = 1780) | S5871274 | Glyma11g15480 |
| GGGGGAAAACACCCATGTAT (SEQ ID = 1781) | TTCCAGAAGACACACCAAGC (SEQ ID = 1782) | S4876163 | Glyma13g19860 |
| CTGTGTGTTTCGCTCCAAGA (SEQ ID = 1783) | GGGAATGGATCCCGAATTAT (SEQ ID = 1784) | S23066904 | Glyma20g02370 |
| TGGGCTTCCTCAATTACACC (SEQ ID = 1785) | GTTGGGATACTGCATTGGCT (SEQ ID = 1786) | S5146307 | Glyma01g22680 |
| GTCCCTGGAGCTGATGGAT (SEQ ID = 1787) | TGGGACTCGATACAATGTGC (SEQ ID = 1788) | S5142129 | Glyma03g27270 |
| AGGAGGTGCCTGGTCTGTTA (SEQ ID = 1789) | ACAACATGGAAACCTGCTCC (SEQ ID = 1790) | BQ613024 | Glyma03g27270 |
| CATGGGGCTCCTTTTTGTTA (SEQ ID = 1791) | TTCATCCAGCTCATGGACAA (SEQ ID = 1792) | S21538774 | Glyma19g01920 |
| GAATTGCTCGGCTCATTTTC (SEQ ID = 1793) | TGAAGGCGAAGAGTCTGACC (SEQ ID = 1794) | S23061205 | Glyma18g08990 |
| GCAAACCAGCTTCTGGAGAG (SEQ ID = 1795) | CGACAATCCTGAACCCAAAT (SEQ ID = 1796) | S5146235 | Glyma02g09060 |
| TAGTGAAAGCACGAGAGCGA (SEQ ID = 1797) | CAAGAACGAAGCTTTGACCC (SEQ ID = 1798) | BE807568 | Glyma04g05820 |
| CGGTTACAATGGGCTTCTGT (SEQ ID = 1799) | CAGGCTGGTGATGTCATTTG (SEQ ID = 1800) | S23061947 | Glyma05g05490 |
| CAACAACCACCTCCACAAAA (SEQ ID = 1801) | CAACACCAATGGAGCTTGTG (SEQ ID = 1802) | S16523441 | Glyma10g36950 |
| TTTCCGTGATTTTCTGACCC (SEQ ID = 1803) | CACCACGATATATGGCAGCA (SEQ ID = 1804) | S4880628 | Glyma11g37390 |
| CTGCATTCTCTGCAACTCCA (SEQ ID = 1805) | TCTGAAATTCGGTGAGGCTT (SEQ ID = 1806) | S22952226 | Glyma16g01370 |
| AACACCTTCAAAGCCACCAC (SEQ ID = 1807) | TGGATGGAACAGTGGCATTA (SEQ ID = 1808) | S5146234 | Glyma16g28250 |
| TGTGGTGTTGCCAGTGGTAT (SEQ ID = 1809) | GAGAAGAACTCGGTGGCAAG (SEQ ID = 1810) | BM519961 | Glyma20g30640 |
| TGATACAGGGAAAGAGAGACGC (SEQ ID = 1811) | GACCTGACCCGACCCAAAT (SEQ ID = 1812) | BI699475 | Glyma20g39410 |
| ACCAGCAAACAAAAACTGGG (SEQ ID = 1813) | CATCACAAACAAGCTGGTGG (SEQ ID = 1814) | BE802758 | Glyma06g08780 |
| CCAGGGATCATAGATGTCGAA (SEQ ID = 1815) | TACAGCACGGAACCACTAGC (SEQ ID = 1816) | S5142330 | Glyma09g32420 |
| TGCAGCTTCACACACAATGA (SEQ ID = 1817) | CTTGGGACTTGTTGAAGGGA (SEQ ID = 1818) | S5146302 | Glyma17g31400 |
| CGCTGGATTGATTCTGGAGT (SEQ ID = 1819) | GCATGCATCTACCACCACAC (SEQ ID = 1820) | S21539810 | Glyma14g08020 |
| AGTTACAATGTTGGCGCCTT (SEQ ID = 1821) | GGAGCTGGTTGAGATGGTGT (SEQ ID = 1822) | S4901474 | Glyma15g05490 |
| TTGTCATCACCCATGAATCG (SEQ ID = 1823) | TTTTGGAAGGCATTTCTGCT (SEQ ID = 1824) | BU549842 | Glyma19g33170 |
| AATTCCCAAGAATCCCTTGC (SEQ ID = 1825) | CCCTCAGTTGGTGCTGATG (SEQ ID = 1826) | S15849836 | Glyma01g05000 |
| GCATTCTATTGAAGAGCGCC (SEQ ID = 1827) | AGCGGTCATGGGTATCAAAG (SEQ ID = 1828) | S5076201 | Glyma03g41270 |
| TCACAGGGTGATTGGTGAAA (SEQ ID = 1829) | ATGCCAACCCAAGATATGGA (SEQ ID = 1830) | S5145495 | Glyma08g40850 |
| AAAACCTGTGTTCACTGGGC (SEQ ID = 1831) | CAGGGCCTATCAGTGCAAAT (SEQ ID = 1832) | S4898136 | Glyma01g06550 |
| AGAAAAAGGTCAAGCGCTCA (SEQ ID = 1833) | AGCGCTTGTTAGGATGAGGA (SEQ ID = 1834) | AI966268 | Glyma01g06550 |
| CAATCTCTCCGCGTTTTCTC (SEQ ID = 1835) | TTGAAGTGCGAACAAGAACG (SEQ ID = 1836) | TC231049 | Glyma01g06870 |
| CTTTCAGCAGCAGCAACAAC (SEQ ID = 1837) | CGGAACATCATTTCTGCTTG (SEQ ID = 1838) | TC207514 | Glyma02g15920 |
| TCCTTGGCTCTGGAAGAGAA (SEQ ID = 1839) | TTTGGATTCTCAGGGTTTGG (SEQ ID = 1840) | BE657634 | Glyma02g39870 |
| AAATTTTGGAAGTGGGGGAC (SEQ ID = 1841) | CCAATCCTGTGGCTGTATAA (SEQ ID = 1842) | S4911583 | Glyma02g39870 |
| CTCTCATCCAAACTGCCTGG (SEQ ID = 1843) | TGCTGACCGATACAAATGGA (SEQ ID = 1844) | BU578846 | Glyma02g47650 |
| TTATCACCGATCCTCATCCC (SEQ ID = 1845) | CAAGATCAAGCCCCATTTGT (SEQ ID = 1846) | S15850879 | Glyma03g31630 |
| TGGCCAAGAGTCAACGACTA (SEQ ID = 1847) | GTGATACACGCATCACGTAAAA (SEQ ID = 1848) | AW507762 | Glyma03g37670 |
| TCTCCTTGATTTCCCTCTATCG (SEQ ID = 1849) | CGCAGGTTGCTGGTTGTTAT (SEQ ID = 1850) | TC231690 | Glyma03g37940 |
| CTGGTTGTATGTGATATCTCGG (SEQ ID = 1851) | ACCTTCATATCGACAGGGCA (SEQ ID = 1852) | S4999395 | Glyma03g37940 |
| TTAATGCCCCTTCTTCAACG (SEQ ID = 1853) | CTGCAGTGAAGTTCGGATCA (SEQ ID = 1854) | TC212079 | Glyma03g38360 |
| TTTCAGCCCCAACTTCAGTC (SEQ ID = 1855) | GAAAGGGAAATCCGTGTCAA (SEQ ID = 1856) | TC209320 | Glyma03g41750 |
| CGCAACAAACACATAGCCAC (SEQ ID = 1857) | CTGCCATTTTCTCACCGATT (SEQ ID = 1858) | TC216813 | Glyma04g08060 |
| TTTACATTGCAACCACCACC (SEQ ID = 1859) | AAGAAAGGGGAACTGTTGGG (SEQ ID = 1860) | S22953062 | Glyma04g08060 |
| GATAACCGTCACTCTGCCGT (SEQ ID = 1861) | CAGCATCTTCCAACACGAGA (SEQ ID = 1862) | TC221320 | Glyma04g39650 |
| AGAAGTGAGGCTATTGGGCA (SEQ ID = 1863) | CCCAGCTCAAGTCACTCTCC (SEQ ID = 1864) | BM144029 | Glyma05g36970 |
| TTGCAGCTTGCGTAATATCG (SEQ ID = 1865) | TGTGTCGTCCATTCGTCATT (SEQ ID = 1866) | S5017551 | Glyma05936980 |
| TCATCTCCTTACTCAGCCGC (SEQ ID = 1867) | AAGGTGGAGGGAGGTTGGT (SEQ ID = 1868) | CA936030 | Glyma06g08120 |
| GCTCCAAACTCATCAACCGT (SEQ ID = 1869) | TTCAAGAGAAAAACCGTGGG (SEQ ID = 1870) | S4909087 | Glyma06g13090 |
| CCATCACCTGATATCCCCAC (SEQ ID = 1871) | ATGACCCAGAGCCAAAAAGA (SEQ ID = 1872) | S21567785 | Glyma06g27440 |
| AAGGTCGCATGAATAAGTTCG (SEQ ID = 1873) | CCCCCTCGAGTTTTTGTTTT (SEQ ID = 1874) | S4883851 | Glyma07g02630 |
| GTTTGGAAACAAAACCGTGG (SEQ ID = 1875) | GGCAACAACACATGGTGAAG (SEQ ID = 1876) | S15852359 | Glyma07g13610 |
| TCAACTGAAAGCTTCGAGCA (SEQ ID = 1877) | GTTTCCATCCATGTCACCCT (SEQ ID = 1878) | TC213679 | Glyma08g01430 |
| TTCTACCCAGTTTTGCACCC (SEQ ID = 1879) | TTGCAGGGCTGCTACTTTCT (SEQ ID = 1880) | TC232713 | Glyma08g02160 |
| AATTCTGGCTCCGTGTTAGC (SEQ ID = 1881) | GCTCCCTTTAATGCCCTTCT (SEQ ID = 1882) | S4904584 | Glyma08g02580 |
| CGATGTGGATGTATTGGACG (SEQ ID = 1883) | TATATACCTGGGGTGCTGCG (SEQ ID = 1884) | TC223475 | Glyma08g15210 |
| GCAAGCTTTTCTCTTTGGGA (SEQ ID = 1885) | ACTCACCCGCTTCAGTTCCT (SEQ ID = 1886) | S5871333; | Glyma08g23380 |
| | | TC225723 | |
| GTTATTACCGGTGCACCCAC (SEQ ID = 1887) | TGAATTTGAATCGTCGCAAG (SEQ ID = 1888) | TC232880 | Glyma09g37930 |
| ACTCCTTTTCAACCCCATCC (SEQ ID = 1889) | GAGGAAATTGAGGGAGGGAC (SEQ ID = 1890) | CF809068 | Glyma09g41050 |
| TCAGGGATCCTCATCCTCAC (SEQ ID = 1891) | TGGATAATATTGTTGGCGCA (SEQ ID = 1892) | S4875903 | Glyma10g03820 |
| GCATCGGCAAATACTTACACAA (SEQ ID = 1893) | CTTGGTCCCATTACTCAATCAA (SEQ ID = 1894) | S21538195 | Glyma10g13720 |
| ACGTACACCGGAGACCACTC (SEQ ID = 1895) | GAAGCAGGAGAGTGACCCAG (SEQ ID = 1896) | TC223128 | Glyma10g37460 |
| TCGGCACGAGAAAACTTCTT (SEQ ID = 1897) | GGGCATGATGTCCTGAAACT (SEQ ID = 1898) | S4897912 | Glyma11g18810 |
| TCCTTCCCAACACAAACACA (SEQ ID = 1899) | TTTCTGGAAAACTCCATCCG (SEQ ID = 1900) | S4983390 | Glyma11g29720 |
| TAAGCTCCTGCCTTCCAGTG (SEQ ID = 1901) | GGTGCTTCTTGCAAAGGTTC (SEQ ID = 1902) | TC220597 | Glyma12g23950 |
| GCGGTGAGGGTGTATCTCTT (SEQ ID = 1903) | CGCGCGTTAATACCACCTAT (SEQ ID = 1904) | S4906707 | Glyma13g00380 |
| CCCAAACCTCTAAGGACAACC (SEQ ID = 1905) | TGACCATGCAATGAAAGAGG (SEQ ID = 1906) | TC208324 | Glyma13g17800 |
| ATTCTGATCTCCCAAGCGAA (SEQ ID = 1907) | TGAGTCATCGCGACTAGACAA (SEQ ID = 1908) | TC222844 | Glyma13g29600 |
| AAGGAAGCAAGTTGAGCGAA (SEQ ID = 1909) | GAGAGGGAGGGAGTGGTTGT (SEQ ID = 1910) | S4873428 | Glyma13g36540 |
| CCACACCTTGCTGACACAGT (SEQ ID = 1911) | ATGGAAGTGATGGCTGCTG (SEQ ID = 1912) | S5052631 | Glyma13g38630 |
| TCTTCCCCACCAACAGCTAC (SEQ ID = 1913) | TGCTCTAACATAACCTGCGG (SEQ ID = 1914) | S4904543 | Glyma13g44730 |
| CAGCTATTGCTTTTGTTCCCA (SEQ ID = 1915) | GAGAAAGAGAGAGAGGGTCCAA (SEQ ID = 1916) | S22953012 | Glyma14g17730 |
| ACAGCCTGAGAAGTTGCGAT (SEQ ID = 1917) | ACTGTCCATTTGGAACACCG (SEQ ID = 1918) | BE820324 | Glyma15g00570 |
| GATTCCCCGTCAACCTCAG (SEQ ID = 1919) | TGAGAGGGTGGAGGTGTAGG (SEQ ID = 1920) | CF807231 | Glyma15g11680 |
| TGAAAAACTTCCCTCTTGTGC (SEQ ID = 1921) | TTTCCATTGCAAACCAAACA (SEQ ID = 1922) | S4909263 | Glyma16g02960 |
| GATCACGAGCCCTCTCTCAC (SEQ ID = 1923) | CCTAAATCCTCAGAGCTGCAC (SEQ ID = 1924) | S4901804 | Glyma17g18480 |
| GAGCCAATTGATCAACACGA (SEQ ID = 1925) | TCACTCTCGGCAGCTTTTCT (SEQ ID = 1926) | BM188198 | Glyma17g33890 |
| GCACTTCGAATTGTCGCTGT (SEQ ID = 1927) | CTCAAACCAAAGTGAAGCCC (SEQ ID = 1928) | S4992221 | Glyma17g33890 |
| AAGCACATTAGATTGCGTCG (SEQ ID = 1929) | TGTGACATCGCCTCGAGTAA (SEQ ID = 1930) | S4925263 | Glyma18g47350 |
| GATGGTTACCGATGGAGGAA (SEQ ID = 1931) | TTGCTTCTTCACATTGCACC (SEQ ID = 1932) | S4874738 | Glyma19g26400 |
| TTGGTCTTCCTCCTTTGTGG (SEQ ID = 1933) | AATTCACCCCAACAACCAAA (SEQ ID = 1934) | S21566010 | Glyma19g40470 |
| TTGCAAAGTTTAGAGACCAA (SEQ ID = 1935) | TGGGTTGACAAATTAGTCCTT (SEQ ID = 1936) | S4864975 | Glyma20g03410 |
| GGACAGGGATGAGGATGAAA (SEQ ID = 1937) | ATACGAGGATCCTATGGGGC (SEQ ID = 1938) | S21568212 | Glyma20g03410 |
| GCAGGAAGGGAATACTGACG (SEQ ID = 1939) | CCTACATTCCAGGCCCAGT (SEQ ID = 1940) | S4971908 | Glyma03g03500 |
| CCCTCAGTCACAGAAACAGC (SEQ ID = 1941) | GCTCTACTGCCTCAAATGGC (SEQ ID = 1942) | TC215832 | Glyma12g10210 |
| GGCACGAGATAAACGGAAGT (SEQ ID = 1943) | TCAGGAGTCTTCCCATCCAG (SEQ ID = 1944) | S4911826 | Glyma13g38750 |
| GGGCTCATTTTCCCCATATT (SEQ ID = 1945) | TATTCAATAGCGCAGCCCTT (SEQ ID = 1946) | S4877093 | Glyma17g12200 |
| TTATCCCAACGCCTTTTCTG (SEQ ID = 1947) | AGGAAGAGCCAAAACACCAA (SEQ ID = 1948) | BGT55046 | Glyma08g23720 |
| TCGTGATGAGAGAGTATCGCTT (SEQ ID = 1949) | TCCGTCCAGACTGCACATAA (SEQ ID = 1950) | S5055124 | Glyma08g23720 |
| AAACCACCCAAGGTGATCTG (SEQ ID = 1951) | TGTCGCGAATCGTATGAGAA (SEQ ID = 1952) | S15940089 | Glyma10g35330 |
| CTGGTGTATCGTGTGCGTCT (SEQ ID = 1953) | AAAGGGAGAGGTTGGTGGTT (SEQ ID = 1954) | BM886879 | Glyma12g30920 |
| CGAACCGAGTGCTTTCACTT (SEQ ID = 1955) | ATGATGCTTCTGGGTAACGG (SEQ ID = 1956) | S5138328 | Glyma12g07510 |
| GAAGGAAGAAACAACGCTCG (SEQ ID = 1957) | CGAACCAGTGTCACTAGCCA (SEQ ID = 1958) | BM095044 | Glyma04g01120 |
| TGCTTCGTTTGCACCTAATG (SEQ ID = 1959) | CGGCCATAGTGTCTCCACTT (SEQ ID = 1960) | CA783495 | Glyma06g01140 |
| AAATGGATCAGCAGAGTGGG (SEQ ID = 1961) | GGGAGGAGTCATCTGTGGAA (SEQ ID = 1962) | CA820031 | Glyma06g02970 |
| CAGGAACAGACATGGCACTG (SEQ ID = 1963) | TGGACAGTTCCTCAGATCCC (SEQ ID = 1964) | S21538405 | Glyma09g14880 |
| GGTGTTGGAACCATAGGCAT (SEQ ID = 1965) | AAGCATTGGAACCAGGTGAG (SEQ ID = 1966) | S22952581 | Glyma11g07930 |
| AGCTGCTTTAAGGAACGTGG (SEQ ID = 1967) | GCTTTCATATGGATGAGCTGC (SEQ ID = 1968) | S4995471 | Glyma11g11850 |
| AGCCAGTAGCCTTTCTGCAA (SEQ ID = 1969) | ACGTGACCTTTTTCATTGCC (SEQ ID = 1970) | S28053803 | Glyma12g05570 |
| AAGGTTGTGTTGCGTCTTCA (SEQ ID = 1971) | AAGGCATAACACATCTCCGC (SEQ ID = 1972) | S5104460 | Glyma13g33420 |
| GCTGAAATTGCAACTGGGAT (SEQ ID = 1973) | AAGGTTGTAAGCAGGCCCTT (SEQ ID = 1974) | S5140118 | Glyma14g36930 |
| TGGTATCCGGCTCATCTTTC (SEQ ID = 1975) | CGGTTCATAACCCTCATGCT (SEQ ID = 1976) | CD405603 | Glyma11g31270 |
| GTGCAAGAGAAACCCTCTGC (SEQ ID = 1977) | CCTAGGGCTTGTGAGTTTGC (SEQ ID = 1978) | BG047435 | Glyma01g04310 |
| TGGATGAAGCAGGATATAGATGG (SEQ ID = 1979) | ATCAACCTACGCACCGCTAC (SEQ ID = 1980) | S5010723 | Glyma01g24820 |
| GCCACTTGTACCGCCTGTTA (SEQ ID = 1981) | GGGGAATTTTCAGGCAACTC (SEQ ID = 1982) | BG362868 | Glyma01g38290 |
| GATCTCAACTTGCCAGCTCC (SEQ ID = 1983) | ACCCAATTGCTGCAGAGAAG (SEQ ID = 1984) | S4908810 | Glyma01g41780 |
| TTACTCCATCGGTCTCTCGAC (SEQ ID = 1985) | GTGAGTTCGGTCTCCGACA (SEQ ID = 1986) | CD405808 | Glyma01g41780 |
| GAGAAGGGGTAGGGATCCAG (SEQ ID = 1987) | CAAGGAGGACATGGAGTTGG (SEQ ID = 1988) | S21537487 | Glyma02g31270 |
| AATGTTTCAAGCAACCAGGC (SEQ ID = 1989) | TTGGCTGTGGAAAGGTTTTT (SEQ ID = 1990) | S21540805 | Glyma02g46270 |
| TCAAGGATGCCTCGGTCAC (SEQ ID = 1991) | TCATGCTGTAGAAGGTGCTGA (SEQ ID = 1992) | TC210774 | Glyma02g46270 |
| TTGGACTTGGAGTTACACCTG (SEQ ID = 1993) | AGAAAAAGAAGCTGAGGTGGTG (SEQ ID = 1994) | AW598570 | Glyma03g33070 |
| AATGCAACCTCGTTTTCGTC (SEQ ID = 1995) | TATGATCCAACCTTGCCCTC (SEQ ID = 1996) | BM086022 | Glyma03g38180 |
| CAATTGCAGAAGGTAGATGAGTC (SEQ ID = 1997) | GCCAATTGTACTGTTTGGTTTG (SEQ ID = 1998) | S21537369 | Glyma03g38180 |
| GGGATTCAAGGTCCACTTCA (SEQ ID = 1999) | GCGAGAGACAGGAGGAAGAA (SEQ ID = 2000) | S23067472 | Glyma03g39120 |
| TAAGCCTAGGCCACGAAGAA (SEQ ID = 2001) | ACCCCAACCTGCACTATCTG (SEQ ID = 2002) | S22953038 | Glyma04g03560 |
| GGGTAACCTCGTCATCAACG (SEQ ID = 2003) | TGGTCCACTCACACAGGAAG (SEQ ID = 2004) | BF324775 | Glyma04g04760 |
| TCCCTCGGCTCAAATATCAC (SEQ ID = 2005) | CCCTTAATAGGGTTGGGCTT (SEQ ID = 2006) | S23070418 | Glyma04g15990 |
| GCCAGTCCAACTGTGACCTT (SEQ ID = 2007) | TCATCGGGCATGAAAGGTAT (SEQ ID = 2008) | AI461128 | Glyma04g16850 |
| GGTCCACCTTCTTCCTCCTC (SEQ ID = 2009) | AAACAGTGCTCTCGGATGCT (SEQ ID = 2010) | S23065601 | Glyma04g36630 |
| GAAAATGGGGTGGCTAACAA (SEQ ID = 2011) | GAGAGAGACACAACCTCGGC (SEQ ID = 2012) | BM527349 | Glyma05g26780 |
| AGAAGCTTGTGGTGGAGGAG (SEQ ID = 2013) | GACCAACAAGGAGCTGGTGT (SEQ ID = 2014) | S5129767 | Glyma05g26990 |
| TTTTCTAGCTACCCTAGCGAAT (SEQ ID = 2015) | GCTGGCTATTAATCCCACGTA (SEQ ID = 2016) | BQ299693 | Glyma05g33590 |
| ATCCTGGCTGCTCATTATGG (SEQ ID = 2017) | CTGTACCCAAAGGAGGTGGA (SEQ ID = 2018) | BM142986 | Glyma05g34280 |
| TTTCCGGACTACTCAGCAGG (SEQ ID = 2019) | TGAGGATTTTCAATCATGGG (SEQ ID = 2020) | S4873409 | Glyma06g04840 |
| CCCACCAAGGTTTGTAATGC (SEQ ID = 2021) | GCAGCACCTGAAATTAGGGA (SEQ ID = 2022) | S23062231 | Glyma06g21730 |
| GTGGTGCAGCTGGGAATAAT (SEQ ID = 2023) | CATGGATGCAATTTCCAATG (SEQ ID = 2024) | S5059623 | Glyma07g01130 |
| CATGGAGTGATCTTGTTGTTGC (SEQ ID = 2025) | CAACAAGCCTTAACGAGACAGA (SEQ ID = 2026) | S15937949 | Glyma07g17810 |
| GGTGATGGCGAGTTGAAAGT (SEQ ID = 2027) | AACCCTTGGAGTTGCTGATG (SEQ ID = 2028) | S4916522 | Glyma08g09970 |
| AGCATCTATCACGGCCAATC (SEQ ID = 2029) | AAAGGCAAAAGAGCCATCAA (SEQ ID = 2030) | S5145792 | Glyma08g13310 |
| CTAGCCACAAGAAGCCCAAG (SEQ ID = 2031) | CCATGCCACAAATTGAACAC (SEQ ID = 2032) | S5045942 | Glyma10g05210 |
| CGAACTCCGTTGGAGAAAAG (SEQ ID = 2033) | AGGCTTGGCAAAAAGTCTCA (SEQ ID = 2034) | S23062194 | Glyma10g05210 |
| AAGCTTCTGCTTTGCCTGAG (SEQ ID = 2035) | TCTCCACTTCAAGGAATATCCA (SEQ ID = 2036) | S5146708 | Glyma10g05850 |
| CACCTCCGTTGTTGTTGTTG (SEQ ID = 2037) | CAAATGGGTTCCACCAGAAG (SEQ ID = 2038) | S21539084 | Glyma10g05880 |
| GGAGTTCGCCTAGTTCCTGA (SEQ ID = 2039) | CTCATAATTCGATGGGTCGC (SEQ ID = 2040) | AI794788 | Glyma10g17510 |
| GGTTGCACTTGACTTGGGTT (SEQ ID = 2041) | AATGTCCTGGTCCCACAAAG (SEQ ID = 2042) | S4993174 | Glyma10g17510 |
| AAGAAAGGCTTTTGCAGCAT (SEQ ID = 2043) | TGAGGACAATTTTTCCCACAC (SEQ ID = 2044) | S21566969 | Glyma10g37780 |
| GGAAGTAACAGCGTTGGAGG (SEQ ID = 2045) | CCCACTCATTCCCCTCACTA (SEQ ID = 2046) | S4913507 | Glyma10g42660 |
| CAAGCTTTGGGAGGACACAT (SEQ ID = 2047) | CTGCTGCCAGAACTCATCAA (SEQ ID = 2048) | BI321317 | Glyma10g43630 |
| CCTCCTGTTAGGGTGGTGAA (SEQ ID = 2049) | AGCTCCACCTCCAGCAGTTA (SEQ ID = 2050) | BG508740 | Glyma10g44160 |
| CAACGATGCCACCAACATAG (SEQ ID = 2051) | TAGCGGTGATAGCAGTGGTG (SEQ ID = 2052) | CA786021 | Glyma12g30270 |
| GTTTGGGACATCATCGTCGT (SEQ ID = 2053) | CGTTGGCATGTGTAAATGATG (SEQ ID = 2054) | AW568213 | Glyma13g40240 |
| TTCATGTGAATGGCTTTGGA (SEQ ID = 2055) | AAGCTTTGCTATTCCGGGTT (SEQ ID = 2056) | S6670395 | Glyma14g13360 |
| CCTTGGATTGGACAACCATC (SEQ ID = 2057) | GACCAGGACCACCACCTCTA (SEQ ID = 2058) | S4964820 | Glyma15g02840 |
| AAATGACAAGCCTTTGTGGC (SEQ ID = 2059) | TGGATGACCTTGTTTCAGCA (SEQ ID = 2060) | S21540601 | Glyma16g06040 |
| TGAAGTTCATGCTCTGCACC (SEQ ID = 2061) | TTGGATGACACTAAAGGGGC (SEQ ID = 2062) | S4993204 | Glyma16g27280 |
| GACCCCAGTGTGATGTTGAA (SEQ ID = 2063) | ATGCCTTTTTGACGAGCAAT (SEQ ID = 2064) | S19678454 | Glyma16g27280 |
| AGGATTTGTGACAAGCGTGG (SEQ ID = 2065) | AGGAACACAAACTCGCCAAT (SEQ ID = 2066) | BU548087 | Glyma17g15140 |
| TTTCAGCAATGGCAGAGCC (SEQ ID = 2067) | AGTGAAGCTTTGGAGGGAGA (SEQ ID = 2068) | BI892530 | Glyma17g15140 |
| GAACCGTCAAGGTTTTTGGA (SEQ ID = 2069) | ACAGTTTCATCGCGATCCTT (SEQ ID = 2070) | BM887582 | Glyma17g33140 |
| ACTCTCAGAATTCCATCGCC (SEQ ID = 2071) | ATCGAGTGTTTGCTTCGCTT (SEQ ID = 2072) | BU964979 | Glyma18g02010 |
| TCGCGGTACTCTTCGAATTT (SEQ ID = 2073) | CAAGCCATTCCCAACCATAA (SEQ ID = 2074) | S23067146 | Glyma18g07330 |
| AGAGCAGTGGCAGTGGAAAT (SEQ ID = 2075) | CACATGATCCACCAAAGCAG (SEQ ID = 2076) | BI424123 | Glyma19g32220 |
| ATAGCACGAGGGTGGTTACG (SEQ ID = 2077) | TGCCATCTTTCCAAACAACA (SEQ ID = 2078) | AW306777 | Glyma19g35740 |
| TCACCTCAGTTGCTTCAACG (SEQ ID = 2079) | AAACACTTTGCATTCCCTGG (SEQ ID = 2080) | BI785592 | Glyma19g36430 |
| TAAGGCCTGAGAGTTTCCGA (SEQ ID = 2081) | CCCACTAACAGAGCAGGAGG (SEQ ID = 2082) | S21540486 | Glyma19g40220 |
| TGAACTGATGTCAGGGTCCA (SEQ ID = 2083) | TAGCGAGACAGACCCACCTT (SEQ ID = 2084) | TC219174 | Glyma02g17260 |
| AATTGGGAAGGGTGTGTGAA (SEQ ID = 2085) | GATTTGGATCGATTCGTGCT (SEQ ID = 2086) | S4915601 | Glyma02g29360 |
| CCGCCATTCCCTTTATTGTA (SEQ ID = 2087) | GGGCCTAAAAACCATGGAAA (SEQ ID = 2088) | S4866216 | Glyma02g39210 |
| TTGTAACCCGATTCTTGGGA (SEQ ID = 2089) | AGTTTCCAGAAAGGCCTGGT (SEQ ID = 2090) | S23067580 | Glyma05g02920 |
| AAAATGCCAAGAGTTGGCTG (SEQ ID = 2091) | TACTTCTGCGAGCATTGTGC (SEQ ID = 2092) | S5128425 | Glyma05g37520 |
| TGATGTGGCTGAAAATGGAG (SEQ ID = 2093) | AAGATTCTTTTCCGGCCATT (SEQ ID = 2094) | S4863815 | Glyma06g18240 |
| CTTGTCACAACATCACCGTGT (SEQ ID = 2095) | TGTTTGCACTGTTCCCAACT (SEQ ID = 2096) | S5129446 | Glyma07g37980 |
| AGTAATCGAACCCCAGACCC (SEQ ID = 2097) | AAACTCTGCCCCTGTAGCAA (SEQ ID = 2098) | CA953058 | Glyma08g16340 |
| TCTCGATTTCATCGCCTTCT (SEQ ID = 2099) | AACCTGCAAGTTTGACCACC (SEQ ID = 2100) | BU546851 | Glyma08g25050 |
| CACAGATATGGAGGCGGTCT (SEQ ID = 2101) | TTTGAAGGCCCTCCCTTATT (SEQ ID = 2102) | S5080459 | Glyma08g36540 |
| TTTTGGCAAAGGCTCTGTCT (SEQ ID = 2103) | CTGCTCAGGCAAACCAGAAT (SEQ ID = 2104) | CA785414 | Glyma08g43270 |
| GATAGATCAGGCTCCTCCCC (SEQ ID = 2105) | TCCTCATGGGAATGGAAAAG (SEQ ID = 2106) | S21566772 | Glyma09g15600 |
| GATAGGACAGCCAGAATGCC (SEQ ID = 2107) | ATGGCAACTCTTCCAGCAAT (SEQ ID = 2108) | BI786323 | Glyma09g38650 |
| TTTTGATGGCAACTGTTCAAAG (SEQ ID = 2109) | ATGGGGTGAGCACAAAAGAG (SEQ ID = 2110) | S5102318 | Glyma10g02540 |
| GAAGATGGCAAGGTCCTTCA (SEQ ID = 2111) | GATTGACCCCATTTGACCAC (SEQ ID = 2112) | S18531023 | Glyma10g31370 |
| GCTCTTCCTCTTTCTGCCCT (SEQ ID = 2113) | AATGCCACTCGCAACAAAG (SEQ ID = 2114) | S23065610 | Glyma10g41530 |
| TCTGATGTCTTTTCAGTTGCG (SEQ ID = 2115) | TGAAGCACCTTCTCAGTCCA (SEQ ID = 2116) | S4924581 | Glyma11g10610 |
| TTCCAGTCTGGGTTCTCCTG (SEQ ID = 2117) | AAGAGCAAACAGCTGCATCA (SEQ ID = 2118) | TC225717 | Glyma12g36600 |
| TGCTCCTGCCTTTGATTCTT (SEQ ID = 2119) | TGTAGCTCCATCTCCTGGCT (SEQ ID = 2120) | TC224861 | Glyma14g01990 |
| CCATGGATGGAGCAGCTGTA (SEQ ID = 2121) | ATAACCAAGAAGCATTGCCA (SEQ ID = 2122) | S4898613 | Glyma14g01990 |
| GATTTTCCCATTGCCTGAGA (SEQ ID = 2123) | GCAGCATGAATTCAGACCACT (SEQ ID = 2124) | S4867817 | Glyma18g47660 |
| GATTCCACTGTTCCCTCCAA (SEQ ID = 2125) | AGGCATAGTAGTCCCTGCCA (SEQ ID = 2126) | BU964406 | Glyma19g27980 |
| TGCTCCTCAAGGAAGGAAAA (SEQ ID = 2127) | GGTCAGGATACCACTGGGTG (SEQ ID = 2128) | CD409339 | Glyma19g32340 |
| GCCAGGTAACATGAAATCCAG (SEQ ID = 2129) | CATTGCCGGAGATGTACAGA (SEQ ID = 2130) | CD408173 | Glyma20g36140 |
| GACCCGACCAACCTTAAACA (SEQ ID = 2131) | TCTTGGGCCAAAGCAAATAC (SEQ ID = 2132) | S4866746 | Glyma20g39160 |
| TGTCATGCGATCGAAATGTT (SEQ ID = 2133) | TTGTGAATTGCATCTCTCGC (SEQ ID = 2134) | CF806129 | Glyma02g38870 |
| TAACCGTAGGTGAACGGCTC (SEQ ID = 2135) | CGAAGACGGAGCAGAAAAGT (SEQ ID = 2136) | CD413483 | Glyma06g06300 |
| AGAGGAGCGAGTCCAATCTG (SEQ ID = 2137) | GAGTAACTGTGCGCAAACGA (SEQ ID = 2138) | S4981738 | Glyma07g02320 |
| AATATGGAACAGAAGCCCCC (SEQ ID = 2139) | CGCGATGGGAAGATTATTGT (SEQ ID = 2140) | CD402050 | Glyma13g01290 |
| GAGGGAGATTTGTGAAGGCA (SEQ ID = 2141) | ACACACGAGCATTGAACTCG (SEQ ID = 2142) | S4948369 | Glyma16g05540 |
| GGATTGCTGTTGTGTCAGGA (SEQ ID = 2143) | TATCGCAGTACCCTCGCTTC (SEQ ID = 2144) | S4912269 | Glyma17g07420 |
| TTCACCCCATGTTTATCGTG (SEQ ID = 2145) | GGTGATGATGGGTTAAGGGA (SEQ ID = 2146) | AW567640 | Glyma19g27240 |
| CCAACCAGCTCTTCTCCAAG (SEQ ID = 2147) | TCTGGCACAGAACAGAGGTG (SEQ ID = 2148) | AW756603 | Glyma19g39460 |
| TTACACTGTTGAACGCAGCC (SEQ ID = 2149) | ATGACCCTTTGAGCACAACC (SEQ ID = 2150) | S21566080 | Glyma20g07050 |
| TGTAGCCTAACCCCTCCCTT (SEQ ID = 2151) | CGTCACATGCTCTTGCAGTT (SEQ ID = 2152) | AW598554 | Glyma20g24940 |
| CACAACACAACAATTCCAACCT (SEQ ID = 2153) | ATTTGCAATATTGTGGGGGA (SEQ ID = 2154) | BU548330 | Glyma16g26140 |
| ATACCGATATGATCGGCGAG (SEQ ID = 2155) | CTTTGAAAGGGGAATGCTGA (SEQ ID = 2156) | BM521216 | Glyma19g27160 |
| TTTGCTTTCAAATGTGGCTG (SEQ ID = 2157) | CTCCACCTGATGCACTTCTG (SEQ ID = 2158) | BI321109 | Glyma09g41790 |
| CCAACCTTTCTGCAGCATTT (SEQ ID = 2159) | CCTGTTCACTCTGACAGGCTC (SEQ ID = 2160) | AW459839 | Glyma02g12080 |
| AACAAGATCCTTGCACCACC (SEQ ID = 2161) | ACTTTAAGCCACCACATGGC (SEQ ID = 2162) | S5127299 | Glyma04g41170 |
| AAACTGTTCTTCGACGGAGC (SEQ ID = 2163) | GCTCCACTTTAACCGTGACC (SEQ ID = 2164) | S21540121 | Glyma06g22800 |
| GGAGGGTCTGAATCCAACTG (SEQ ID = 2165) | GACCCGAAACCAAATTCAAA (SEQ ID = 2166) | S34534192 | Glyma08g20840 |
| GGCTTGCATTGAATGGTTTT (SEQ ID = 2167) | CTATATGGGCAACACTGGGG (SEQ ID = 2168) | S5143054 | Glyma09g37170 |
| TGCTGGTTCGTACCCTTTTC (SEQ ID = 2169) | ACCGATGGCATCTGAGAAAC (SEQ ID = 2170) | BI497850 | Glyma12g06880 |
| CTCTAGCTCCACCACGAACC (SEQ ID = 2171) | AAACCTTGGGAAAGGAACAC (SEQ ID = 2172) | S34534190 | Glyma13g24600 |
| TGCCAAAAGGGAACTGAAAC (SEQ ID = 2173) | CATCACCCCCAGTTTCCTC (SEQ ID = 2174) | S23070950 | Glyma15g02620 |
| TGACCCAAACCTATGTGCAA (SEQ ID = 2175) | GGCATTATGCTGTTGAGGGT (SEQ ID = 2176) | S34534176 | Glyma15g07730 |
| TGTTCCACTTGATCAGCAGC (SEQ ID = 2177) | GGTGGTGGCAGAGTTTTGTT (SEQ ID = 2178) | S4932109 | Glyma16g02550 |
| CATTTCCCGGTGTTGAAATC (SEQ ID = 2179) | CATTGCGTCTTCTGGAGTCA (SEQ ID = 2180) | BE657938 | Glyma16g26030 |
| AGCACCTTCCAACAACAACC (SEQ ID = 2181) | CCATGTATAGGGCCAAGGAA (SEQ ID = 2182) | S34534182 | Glyma17g10920 |
| CCTCAAGGAAGAAGGAACCC (SEQ ID = 2183) | GGTTCGGTAGCTCAGCAAAG (SEQ ID = 2184) | S34534187 | Glyma17g21540 |
| CTAGGCAACGAGCCAAAAAG (SEQ ID = 2185) | TATGGTGACTACTCGCACGC (SEQ ID = 2186) | S5143416 | Glyma15g09330 |
| TGATGATCCTGGAGGAAAGG (SEQ ID = 2187) | ACTCTGTGCAATGCTTGTGG (SEQ ID = 2188) | BQ453782 | Glyma01g10390 |
| GCTTCCCGGTTTTTGAATTT (SEQ ID = 2189) | CCCACTGAAACAGGTCCATT (SEQ ID = 2190) | TC234963 | Glyma02g05710 |
| ATTACGGGAAAGTGCGACTG (SEQ ID = 2191) | TCCGCAACCATAATTGTGAC (SEQ ID = 2192) | BE820520 | Glyma02g07850 |
| TGAAGAAAGAGGAGGAGCCA (SEQ ID = 2193) | GCTTTCAAGGACTGAGACCG (SEQ ID = 2194) | CA799894 | Glyma02g08150 |
| AAAGAAACGGGCATATGGTG (SEQ ID = 2195) | GCCTTTCCATCATTCTCCAC (SEQ ID = 2196) | S4925538 | Glyma03g27250 |
| GGGTAATTTGGGGGAAAAGA (SEQ ID = 2197) | TATGTTCCGTGGCGTACAAA (SEQ ID = 2198) | S4864621 | Glyma04g01090 |
| CACGCGATGTTTGGCTACTA (SEQ ID = 2199) | GAGGACGGACCGTATGTGAC (SEQ ID = 2200) | S4872958 | Glyma06g01110 |
| GTCTTCAGCTCCTCCTCGG (SEQ ID = 2201) | TCCCCAGTGATCCTCATTTC (SEQ ID = 2202) | S23071239 | Glyma07g01960 |
| CTTCCTCAGGGAACAGTCCA (SEQ ID = 2203) | GAGAGGAGTCTTGGTGGTGC (SEQ ID = 2204) | S4885901 | Glyma07g37190 |
| GTTGCACCCAGAAAATGCTT (SEQ ID = 2205) | CAGGCATTGCATAGGGTCTT (SEQ ID = 2206) | S4897423 | Glyma11g20480 |
| GTTGCACCCAGAAAATGCTT (SEQ ID = 2207) | CAGGCATTGCATAGGGTCTT (SEQ ID = 2208) | BE556639 | Glyma11g20480 |
| TGGAGATTTGATGAAGCCAA (SEQ ID = 2209) | GCACTCAAACTGCCACAAGA (SEQ ID = 2210) | BE658870 | Glyma12g29730 |
| CCCACACTTTTTGGTCCTCA (SEQ ID = 2211) | TTAGGAAAGGGGAGGGAAAA (SEQ ID = 2212) | S5142472 | Glyma13g00200 |
| GGGCTCGTAGGTAACGTCAG (SEQ ID = 2213) | GTCATAGCCGGCGAATTAAG (SEQ ID = 2214) | S4875857 | Glyma13g40020 |
| TGGAATTCGACAAAGGAAGG (SEQ ID = 2215) | GCTATGCAACGTGTTTCCCT (SEQ ID = 2216) | S5061040 | Glyma15g18380 |
| GAGTGGCAGGATAGTCCAGG (SEQ ID = 2217) | CTCTCTCCTTATCCGCTCCC (SEQ ID = 2218) | S5019221 | Glyma17g06290 |
| GCTAGCTTCTGGGGAGCCTA (SEQ ID = 2219) | CAGGTTGTGAGGCATTTTGA (SEQ ID = 2220) | BU082623 | Glyma20g32050 |
| CCAGAGTTGGCTGTTCCATT (SEQ ID = 2221) | AGCTTCCTCAGTCAAATGTGC (SEQ ID = 2222) | S23064229 | Glyma09g10010 |
| ACTGGTTTGCCACAAGGAAC (SEQ ID = 2223) | TCCCGAAGGAAAGCACTCTA (SEQ ID = 2224) | S5141720 | Glyma03g31820 |
| CCTTGAGCTGAGTTCTGGCT (SEQ ID = 2225) | GGTTTTCATGATGACCCTGG (SEQ ID = 2226) | S22951753 | Glyma02g10480 |
| CATCGTCATCTTGATCGTCC (SEQ ID = 2227) | AAGTCCAGCTCTAAGCAGCG (SEQ ID = 2228) | S23061682 | Glyma07g04040 |
| ACAAGGCTGATAGGAAGCGA (SEQ ID = 2229) | TTCCTTGTTTCTTGGCCATC (SEQ ID = 2230) | S4883098 | Glyma14g11400 |
| GCAACAGATGTCAAATAGCCG (SEQ ID = 2231) | AAGCTTTACAAACCCATGACG (SEQ ID = 2232) | CF808329 | Glyma19g17460 |
| TTTTAATGGGGTCTGGCAAC (SEQ ID = 2233) | ACGCGTTAGTTCTGCTTCGT (SEQ ID = 2234) | CF808357 | Glyma07g00230 |
| GTTATCAAAAGGACCGTGGC (SEQ ID = 2235) | TTGCCTTGCTTCCTTGTTCT (SEQ ID = 2236) | AW102412 | Glyma15g00250 |
| GAGGCCTCCAATGTAATCCA (SEQ ID = 2237) | TCTCTTCCTTGGGAAGCAAC (SEQ ID = 2238) | S5079445 | Glyma02g47850 |
| TCTTCTTGTGGTGCTTGTGC (SEQ ID = 2239) | GTTGCGGTAACCACAGGAAT (SEQ ID = 2240) | BF066816 | Glyma07g34890 |
| CTTTGGAGATCCCATCATGC (SEQ ID = 2241) | CGTTGAGCTTCTGGTGGAAT (SEQ ID = 2242) | BI786075 | Glyma20g02690 |
| GCGCACATTGTTCTGCTTTA (SEQ ID = 2243) | TCCTTGCTCAAGTTCAACCA (SEQ ID = 2244) | BU550961 | Glyma01g43000 |
| TCACGGTTCGTACTGACGAG (SEQ ID = 2245) | AGTGCTCCACCCATTGTTGT (SEQ ID = 2246) | S4882921 | Glyma03g02930 |
| CAATGCTGCGTCTCACTTGT (SEQ ID = 2247) | CATACATGAATGGGGCCTCT (SEQ ID = 2248) | S21537821 | Glyma04g41500 |
| CTACCACAACTAGGAGCCGC (SEQ ID = 2249) | CATTATCACGGCTTGCAGAA (SEQ ID = 2250) | BU550136 | Glyma05g01100 |
| CAATGCCGATTACTCTCCGT (SEQ ID = 2251) | GAGACGGAACCTCCGAGTCT (SEQ ID = 2252) | S6674973 | Glyma05g36110 |
| TTTACAGTTCCAGCACAGCG (SEQ ID = 2253) | ATTATGCAAGAGAATGCCCG (SEQ ID = 2254) | S23064088 | Glyma06g01090 |
| AGGTCACGGGAGGAAGATTT (SEQ ID = 2255) | GAGATGGGTGCTAGGCATGT (SEQ ID = 2256) | S21567496 | Glyma06g34960 |
| TGAAACTTCCAGGCCAAAAC (SEQ ID = 2257) | AGCGAAATTCGGGAAAGACT (SEQ ID = 2258) | S4865156 | Glyma07g27820 |
| AAATAGGGGCATTGATGACG (SEQ ID = 2259) | TTCCAATCCCGGTCCATAG (SEQ ID = 2260) | S4934838 | Glyma08g13630 |
| ACATTCATGCCCCCATCTAA (SEQ ID = 2261) | CGCAACACAACATATGCTCC (SEQ ID = 2262) | S4865951 | Glyma08g13630 |
| CATCTCCAACGTCTCGGTTT (SEQ ID = 2263) | CCTGCAAAGAAGCTTGATGA (SEQ ID = 2264) | S4877743 | Glyma08g36700 |
| AGACCAGTTTTGGCATTGAGA (SEQ ID = 2265) | TTCCAAGCGTGTTTACCAGTC (SEQ ID = 2266) | S23072300 | Glyma08g40840 |
| TTGAGCTAGGTTTGACGGCT (SEQ ID = 2267) | TGGATTTGTCCAAGGTGTGA (SEQ ID = 2268) | BI094989 | Glyma09g37750 |
| TGGCATCAAAAAGGAGAACA (SEQ ID = 2269) | TGAATGCTGGCATCGTAAAG (SEQ ID = 2270) | S5142209 | Glyma10g05910 |
| TATTGGTCCAGTTTTGGGGA (SEQ ID = 2271) | CAACCTTCCAATATCCCTGG (SEQ ID = 2272) | BM178746 | Glyma10g21950 |
| TGCCAGTCAGGATCAGTTTG (SEQ ID = 2273) | CCCAGATAGCATTGAAGGGA (SEQ ID = 2274) | BE346270 | Glyma10g41540 |
| ACGTGACCATAACAACGGGT (SEQ ID = 2275) | GTGCACCGTTGACAAAGCTA (SEQ ID = 2276) | BF009919 | Glyma10g41870 |
| GGGAGGCCATACTCATCAGA (SEQ ID = 2277) | AACTCAGGTGGATGATTCGC (SEQ ID = 2278) | BI315918 | Glyma11g33420 |
| CAATTACACCGAGCATCACG (SEQ ID = 2279) | ATCATCGCTCATCGTGTCAG (SEQ ID = 2280) | S4876881 | Glyma12g30920 |
| TCTCTCCCGCTAAGGTACGA (SEQ ID = 2281) | ACCATTGCATCCAACAATGA (SEQ ID = 2282) | S5144973 | Glyma13g19790 |
| TCCCCAAGGAAGCGTAAATA (SEQ ID = 2283) | ACGTTCGGCTACATCAAAGC (SEQ ID = 2284) | S4980807 | Glyma13g41450 |
| TTAATTGCTGAGCAGGGACC (SEQ ID = 2285) | TTGCAGCAGTGCGATAATTC (SEQ ID = 2286) | S4891868 | Glyma13g41590 |
| TCTGGCTCTCTTGGAATTGG (SEQ ID = 2287) | GATCGGGTGATAGTTCACGG (SEQ ID = 2288) | BU546053 | Glyma17g37430 |
| GGCTTGCATCTTTTGGTTCT (SEQ ID = 2289) | TCCCTCATCTGCAATTTTCC (SEQ ID = 2290) | AI748637 | Glyma18g15520 |
| AGTGCCTCCTCTGCTATGGA (SEQ ID = 2291) | CAAGCAATTGAAGCACTGGA (SEQ ID = 2292) | S6669987 | Glyma19g32340 |
| TGTTTTGTTGGCATGGAGAA (SEQ ID = 2293) | AGCTGAAACTACCTCGCCAA (SEQ ID = 2294) | BM526462 | Glyma19g39460 |
| TCTCATCCTGTTTTCTGCCC (SEQ ID = 2295) | TGACATCCTTGACGTGGAAA (SEQ ID = 2296) | S21700432 | Glyma19g39460 |
| TCTCCTCGGTTAAAGGGGTT (SEQ ID = 2297) | GCACCCAGTATCGCAGTGTA (SEQ ID = 2298) | BM954606 | Glyma20g29060 |

### Example 3

### Tissue Specific Transcription Factors in Soybean

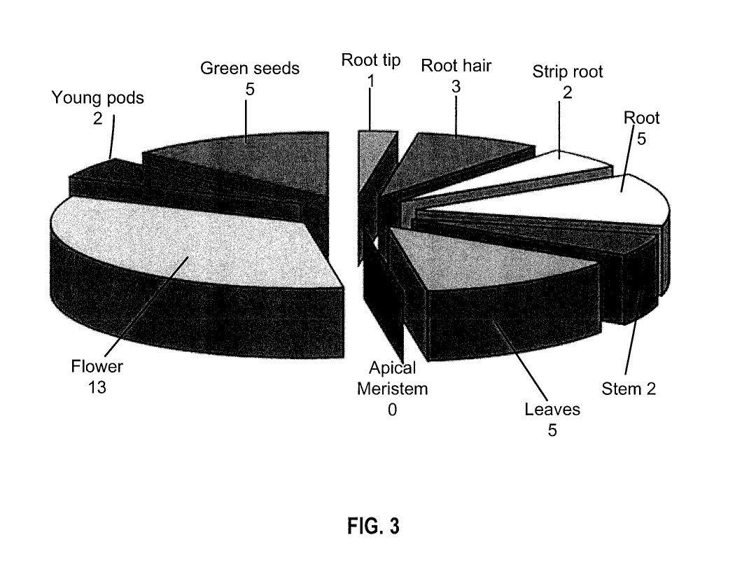

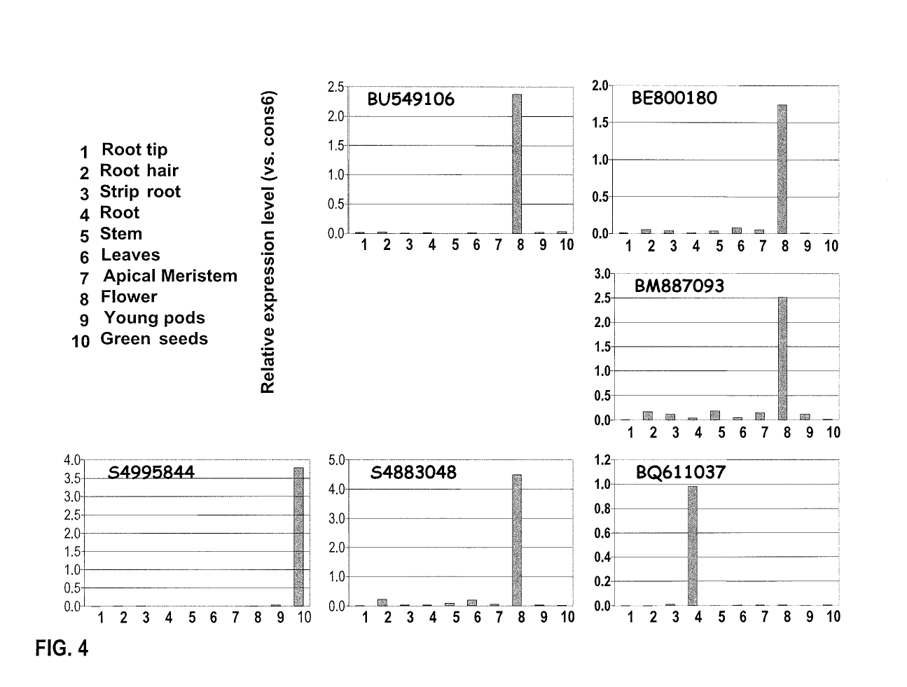

The primers in the primer library described in Example 2 were used to quantitate TF gene expression in 10 tissues from soybean plants. Briefly, soybean strain Williams 82 was grown under normal conditions. RNA samples from 10 different tissues were prepared as described in Example 7 and in U.S. patent application Ser. No. 12/138,392. cDNA were prepared from these RNA samples by reverse transcription. The cDNA samples thus obtained were then used as templates for PCR using primer pairs specific for soybean TFs. The PCR products of each TF gene in different tissues were quantitated and the results are summarized in Table 2. FIG. 3 summarizes a total of 38 TFs found to be expressed at much higher levels in one soybean tissue than its expression levels in 9 other tissues tested. The detailed expression levels of all these TFs are shown in Table 2. FIG. 4 shows the expression pattern of a number of representative TFs. These tissue specific TF genes may play a specific role in the development and function of the particular tissue in which they are highly expressed.

TABLE 2
Tissue specific expression of soybean transcription factors (expression levels are
relative to Cons6)

| | Gene | | | | | |
|---|---|---|---|---|---|---|
| | annotation | | Root | Strip | | |
| ID number | number | Root tip | hair | root | Root | Stem |
| AW831868 | Glyma12g34510 | 0.000377 | 0.000913 | 0.001047 | 0.025711 | 0.001901 |
| BE058570 | Glyma10g41930 | 0.006345 | 0.032269 | 0.007563 | 0.002613 | 0.007938 |
| BE800180 | Glyma16g04740 | 0.006846 | 0.053484 | 0.040451 | 0.013657 | 0.03417 |
| BI469606 | Glyma16g25250 | 0.006882 | 0.000671 | 0.000388 | 0.011848 | 0.017494 |
| BI971027 | Glyma16g04410 | 0.022791 | 1.303916 | 0.052251 | 0.099274 | 0.004044 |
| BM887093 | Glyma04g40960 | 0.007407 | 0.16902 | 0.124614 | 0.03937 | 0.188003 |
| BQ080756 | Glyma03g31940 | 0.00101 | 0.00664 | 0.003759 | 0.124583 | 0.001814 |
| BQ611037 | Glyma03g28630 | 0.000398 | 0.000386 | 0.010116 | 0.979969 | 0.000673 |
| BU549106 | Glyma04g02980 | 0.01402 | 0.019978 | 0.003652 | 0.009667 | 1.98E−06 |
| BU550564 | Glyma02g44040 | 1.98E−06 | 1.98E−06 | 1.98E−06 | 1.98E−06 | 1.98E−06 |
| BU550961 | Glyma01g43000 | 0.003684 | 0.002649 | 0.008877 | 0.01521 | 0.005019 |
| BU761035 | Glyma15g37270 | 1.98E−06 | 1.98E−06 | 1.98E−06 | 1.98E−06 | 0.000283 |
| CA938036 | Glyma20g34420 | 1.98E−06 | 1.98E−06 | 1.98E−06 | 0.00018 | 1.98E−06 |
| CF806953 | Glyma10g36760 | 0.004128 | 0.01162 | 0.002918 | 0.014551 | 0.001365 |
| S17640718 | Glyma06g26610 | 0.004416 | 0.473948 | 0.003488 | 0.004315 | 0.004902 |
| S21537044 | Glyma18g29400 | 0.034376 | 0.008795 | 0.018193 | 0.003953 | 0.005454 |
| S21537813 | Glyma06g01300 | 0.070762 | 0.00725 | 0.115771 | 0.288467 | 0.162836 |
| S21539810 | Glyma14g08020 | 0.138422 | 0.196741 | 0.206804 | 0.080272 | 0.118622 |
| S22336596 | Glyma06g02990 | 0.000506 | 0.001179 | 0.00017 | 0.001694 | 0.001099 |
| S4862200 | Glyma03g08270 | 1.98E−06 | 1.98E−06 | 1.98E−06 | 1.98E−06 | 3.85E−05 |
| S4864621 | Glyma04g01090 | 1.98E−06 | 1.98E−06 | 4.65E−05 | 1.98E−06 | 1.98E−06 |
| S4866216 | Glyma02g39210 | 1.98E−06 | 1.98E−06 | 1.98E−06 | 1.98E−06 | 1.98E−06 |
| S4873428 | Glyma13g36540 | 0.287638 | 5.152291 | 0.209787 | 0.583371 | 0.096919 |
| S4874772 | Glyma07g33510 | 0.000897 | 0.001974 | 0.00094 | 0.005291 | 0.000768 |
| S4878382 | Glyma15g10370 | 0.012597 | 1.98E−06 | 1.98E−06 | 1.98E−06 | 1.98E−06 |
| S4883048 | Glyma16g04740 | 0.01051 | 0.22375 | 0.029437 | 0.027897 | 0.088106 |
| S4883295 | Glyma17g36490 | 1.98E−06 | 1.98E−06 | 1.98E−06 | 1.98E−06 | 1.98E−06 |
| S4891301 | Glyma07g04210 | 0.000887 | 1.98E−06 | 0.000688 | 0.008373 | 0.012137 |
| S4901892 | Glyma07g04200 | 1.98E−06 | 1.98E−06 | 1.98E−06 | 1.98E−06 | 1.98E−06 |
| S4906707 | Glyma13g00380 | 1.98E−06 | 1.98E−06 | 1.98E−06 | 1.98E−06 | 1.98E−06 |
| S4912396 | Glyma07g21160 | 1.98E−06 | 1.98E−06 | 1.98E−06 | 1.98E−06 | 1.98E−06 |
| S4913107 | Glyma04g05500 | 1.98E−06 | 1.98E−06 | 3.98E−05 | 1.98E−06 | 1.98E−06 |
| S4937572 | Glyma13g39990 | 1.98E−06 | 1.98E−06 | 1.98E−06 | 1.98E−06 | 1.98E−06 |
| S4989510 | Glyma08g24340 | 0.042589 | 0.09655 | 0.041722 | 0.060124 | 0.048579 |
| S4995844 | Glyma08g47240 | 0.001913 | 0.012798 | 0.007723 | 9.63E−05 | 6.73E−05 |
| S5045510 | Glyma01g04610 | 1.98E−06 | 1.98E−06 | 1.98E−06 | 1.98E−06 | 1.98E−06 |
| S5132128 | Glyma05g22860 | 0.008098 | 0.004085 | 0.025675 | 0.325942 | 0.031254 |
| TC229552 | Glyma07g32380 | 1.98E−06 | 0.000806 | 1.98E−06 | 0.002534 | 0.011291 |

| | | | | | | Tissue |
|---|---|---|---|---|---|---|
| | | | | | | with the |
| | | Apical | | Young | Green | highest |
| ID number | Leaves | meristem | Flower | pod | seed | expression |
| AW831868 | 0.000453 | 0.001683 | 0.000788 | 0.000963 | 0.000547 | root |
| BE058570 | 0.001846 | 0.006939 | 0.44787 | 0.010157 | 0.010481 | flower |
| BE800180 | 0.07513 | 0.05112 | 1.741048 | 0.010309 | 0.002802 | flower |
| BI469606 | 0.357805 | 0.002047 | 0.024918 | 0.005017 | 0.00083 | leaves |
| BI971027 | 0.019503 | 0.004129 | 0.012126 | 0.002966 | 0.004464 | root hair |
| BM887093 | 0.047448 | 0.148805 | 2.518399 | 0.118856 | 0.010943 | flower |
| BQ080756 | 0.001012 | 0.000118 | 0.00584 | 0.003366 | 0.001692 | root |
| BQ611037 | 0.001543 | 0.001235 | 0.003832 | 0.000636 | 0.003859 | root |
| BU549106 | 0.011153 | 0.000713 | 2.374515 | 0.020434 | 0.034092 | flower |
| BU550564 | 1.98E−06 | 1.98E−06 | 0.000213 | 1.98E−06 | 1.98E−06 | flower |
| BU550961 | 0.000521 | 0.002986 | 0.000785 | 0.004731 | 0.137695 | green seed |
| BU761035 | 1.98E−06 | 1.98E−06 | 1.98E−06 | 1.98E−06 | 1.98E−06 | stem |
| CA938036 | 1.98E−06 | 1.98E−06 | 1.98E−06 | 1.98E−06 | 1.98E−06 | root |
| CF806953 | 0.000748 | 0.000924 | 0.188744 | 0.00106 | 0.007963 | flower |
| S17640718 | 0.002196 | 0.007197 | 0.009113 | 0.001554 | 0.001936 | root hair |
| S21537044 | 0.002606 | 0.01036 | 0.003158 | 0.012512 | 0.706535 | green seed |
| S21537813 | 0.083595 | 0.041227 | 0.134828 | 39.06024 | 0.117816 | young pods |
| S21539810 | 0.021762 | 0.069847 | 0.046511 | 69.95437 | 0.023965 | young pods |
| S22336596 | 0.000857 | 0.001766 | 0.458955 | 0.002108 | 0.003727 | flower |
| S4862200 | 1.98E−06 | 1.98E−06 | 1.98E−06 | 1.98E−06 | 1.98E−06 | stem |
| S4864621 | 1.98E−06 | 1.98E−06 | 1.98E−06 | 1.98E−06 | 1.98E−06 | strip root |
| S4866216 | 1.98E−06 | 1.98E−06 | 3.76E−05 | 1.98E−06 | 1.98E−06 | flower |
| S4873428 | 0.162969 | 0.04782 | 0.249838 | 0.051913 | 0.055284 | root hair |
| S4874772 | 0.279323 | 0.000438 | 0.005084 | 0.000848 | 0.001814 | leaves |
| S4878382 | 1.98E−06 | 1.98E−06 | 1.98E−06 | 1.98E−06 | 1.98E−06 | root tip |
| S4883048 | 0.209528 | 0.057981 | 4.490488 | 0.030216 | 0.006497 | flower |
| S4883295 | 0.000243 | 1.98E−06 | 1.98E−06 | 1.98E−06 | 1.98E−06 | leaves |
| S4891301 | 0.000356 | 0.002711 | 0.003744 | 0.021872 | 0.369057 | green seed |
| S4901892 | 1.98E−06 | 1.98E−06 | 3.83E−05 | 1.98E−06 | 1.98E−06 | flower |
| S4906707 | 1.98E−06 | 1.98E−06 | 4.29E−05 | 1.98E−06 | 1.98E−06 | flower |
| S4912396 | 1.98E−06 | 1.98E−06 | 0.00044 | 1.98E−06 | 1.98E−06 | flower |
| S4913107 | 1.98E−06 | 3.2E−06 | 1.98E−06 | 1.98E−06 | 2.89E−06 | strip root |
| S4937572 | 1.98E−06 | 1.98E−06 | 2.54E−05 | 1.98E−06 | 1.98E−06 | flower |
| S4989510 | 0.032361 | 0.044497 | 0.035619 | 0.04467 | 1.037795 | green seed |
| S4995844 | 0.000194 | 0.000888 | 0.00123 | 0.03702 | 3.785746 | green seed |
| S5045510 | 3.7E−05 | 1.98E−06 | 1.98E−06 | 1.98E−06 | 1.98E−06 | leaves |
| S5132128 | 0.002617 | 0.023895 | 0.007964 | 0.008026 | 0.015074 | root |
| TC229552 | 2.641493 | 0.040778 | 0.279462 | 0.054674 | 0.129584 | leaves |

The tissue specific expression of some of these TFs was confirmed by creating a transcriptional fusion with GUS (i.e., β-glucosidase) or GFP (green fluorescent protein) reported genes. The coding regions of the reporter gene was cloned under control of the promoter of the tissue specific TF gene as described below.

Briefly, the Gateway system by Invitrogen Inc. (Carlsbad, Calif.) was used to clone promoter upstream to the GFP and GUS cDNAs. A 2 kb DNA fragment 5′ to the first codon of the bHLH gene was identified by mining genomic sequences available on Phytozome website (http://www.phytozome.net/soybean.php). Through two independent PCR reactions, AttB sites at the extremities of the promoter sequences were created. Genomic DNA from the soybean strain Williams 82 was used as template for PCR. Using the Gateway® BP Clonase® II enzyme mix, the promoter fragment was introduced first into the pDONR-Zeo vector (Invitrogen, Carlsbad, Calif.) then into pYXT1 or pYXT2 destination vectors using the Gateway® LR Clonase® II enzyme mix (Invitrogen, Carlsbad, Calif.). pYXT1 and pYXT2 were destination vectors carrying the GUS and GFP reporter genes respectively (Xiao et al., 2005).

A. rhizogenes (strain K599) was transformed by electroporation with bHLHpromoter-pYXT1 and bHLHpromoter-pYXT2 vectors. Soybean hairy root transformation was carried out essentially as described by Taylor et al. (2006). Briefly, two-week old soybean shoots were cut between the first true leaves and the first trifoliate and placed into rock-wall cubes (Fibrgro, Sarnia, Canada). Each shoot was inoculated with 4 ml of A. rhizogenes (OD₆₀₀=0.3) and then allowed to dry for approximately 3 days (23° C., 50% humidity, long day conditions) before watering with deionized water. After one week, the plants were transferred to pots with vermiculite:perlite mix (3:1) wetted with nitrogen-free plant nutrient solution (Lullien et al., 1987). One week later, the shoots were transferred to the green house (27° C., 20% humidity, long day conditions). Two weeks after vermiculite-perlite transfer, the shoots were inoculated with B. japonicum (10 ml, OD₆₀₀=0.08).

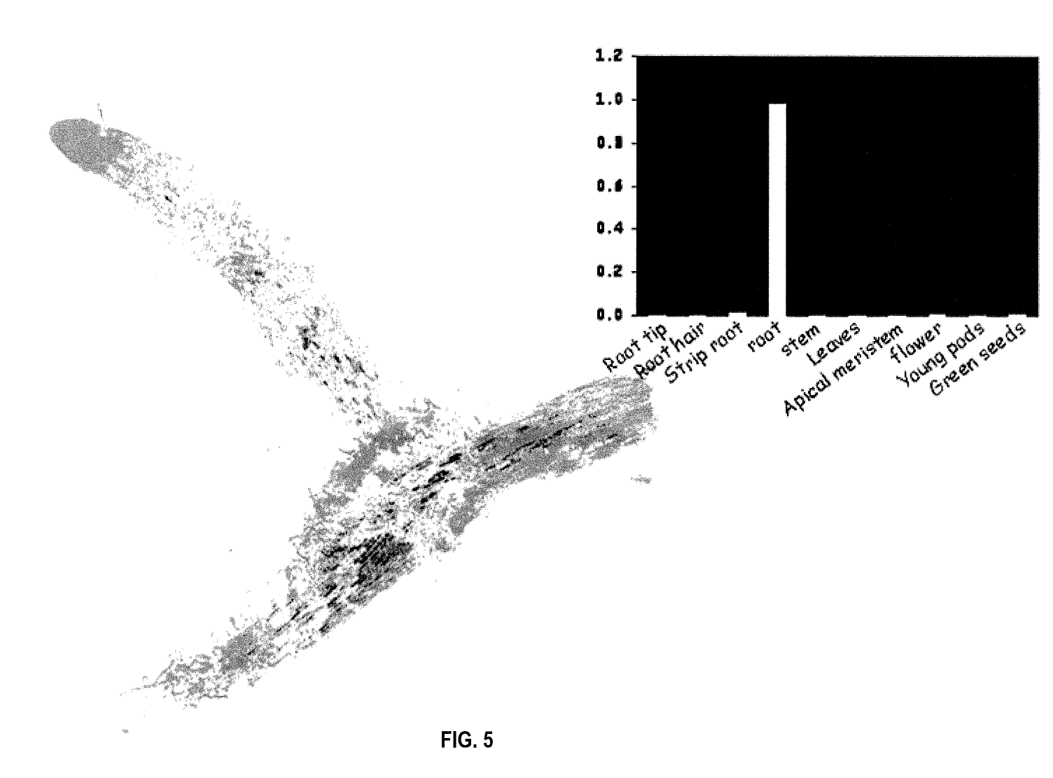

FIG. 5 shows the protein localization of the bHLH TF gene (Glyma03g28630) in mature root cells as indirectly shown by the localization of the reporter proteins, namely, GUS and GFP. The inset is a bar chart showing the tissue specific expression of the bHLH gene (FIG. 5).

### Example 4

### Soybean Transcription Factors Regulated by Different Seed Developmental Stages

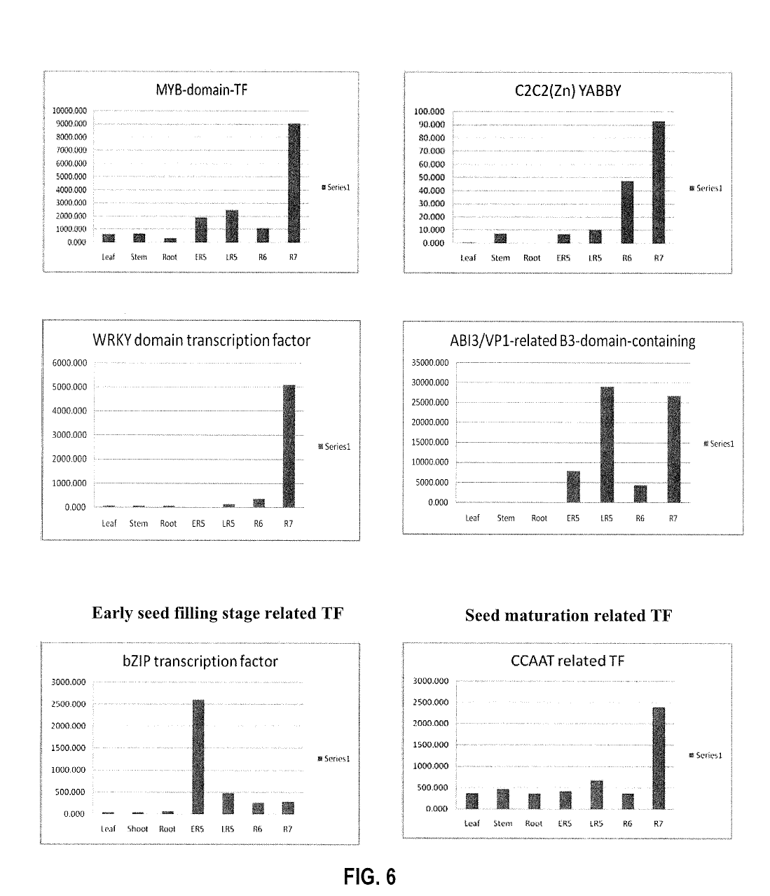

In order to identify soybean TF genes whose expression levels are regulated at different seed developmental stages, soybean tissues including roots, leaves, stems and seeds were harvested and RNA extracted. qRT-PCR was performed as described in Examples 7-9 and in U.S. patent application Ser. No. 12/138, 392 to determine the expression levels of each TF at different seed developmental stages, ER5 (early R5 stage-R5 starting of seed filling), LR5 (late R5 stage-seed filing ongoing), R6 (seed filling stage), and R7 (maturation stage) and R8 matures seed stage. TF Genes that showed stage specific expression during seed development are termed “Transcription Factors Implicated in Seed Development” (TFISD). Examples of TFISD include, for example, Myb, C2C2, bZip, CCAAT binding, DOF, etc. FIG. 6 shows the relative expression levels some of the TFISD genes at ER5, LR5, R6, and R7 stages as compared to the expression levels in leaf, stem and root tissues.

Further functional investigation of these TFISDs will help to understand the mechanisms regulating seed filling and seed composition. These soybean TFISDs, such as bZip and CCAAT, are overexpressed in Arabidopsis thaliana under the control of inducible or constitutive promoters. The expression levels of various genes implicated in seed development are determined to help elucidate which downstream genes are regulated by a TFISD. The filling or composition of the seeds and other characteristics of the seeds are also examined to establish the relationship between the expression of a TFISD and seed development.

In another aspect, the DNA elements responsible for the stage specific expression of a TFISD during seed development are determined using various reporter genes as described above. These DNA elements include but are not limited to promoters, enhancers, attenuators, methylation sites etc. Structural or functional genes are placed under control of the DNA elements of the soybean TFISDs such that they are expressed at specific stage during seed development. The structural or functional genes may be from soybean or other plants that have been identified to control seed composition, such as protein and/or oil content.

### Example 5

### Soybean Transcription Factors Implicated in Flood Resistance

Some soybean strains are naturally more resistant to flooding than others. To identify soybean genes that may confer upon a plant flood resistant phenotype, the gene expression of two soybean strains are profiled. One strain, PI 408105A (PI—Plant introduction), is flooding stress tolerant; the other strain, S99-2281 (Breeding line), is flooding stress sensitive.

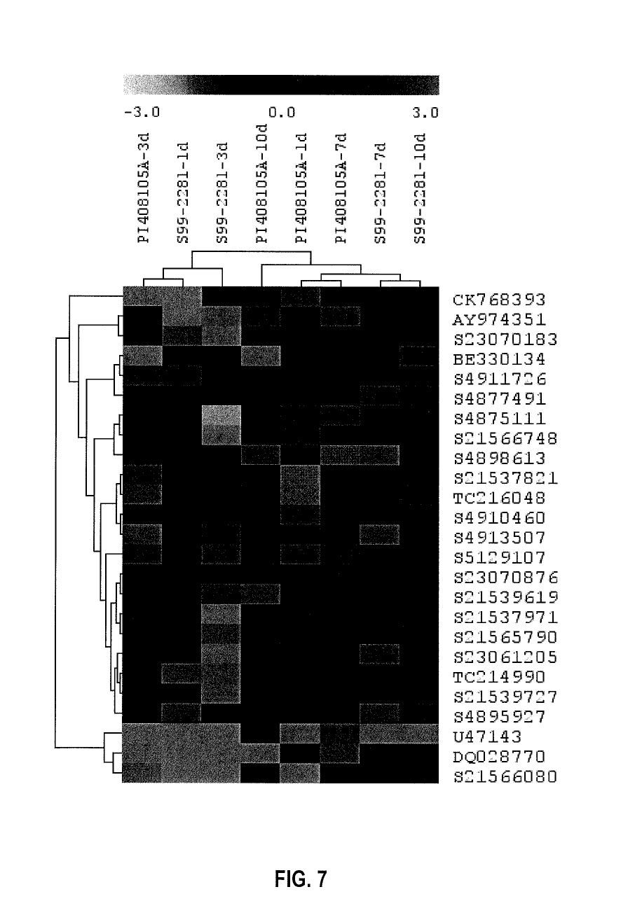

The two soybean strains were grown under normal conditions and water was introduced to flood the plants. Tissues samples were collected at Day 1, Day 3, Day 7 and Day 10 post flooding. Microarray profiling was used to determine the expression levels of all genes across the entire genome as described above. FIG. 7 shows a representative result of this study showing some of the genes that have different expression pattern between the flood tolerant strain and the flood sensitive strain.

### Example 6

### Soybean Transcription Factors Implicated in Root Nodule Development

The expression patterns of soybean regulatory genes regulated during nodule development were studied using qRT-PCR. Expression of 126 soybean TF genes were profiled to identify soybean TFs that are upregulated or downregulated during root nodule development. Table 3 lists the changes of expression levels for these 126 genes recorded at 4 days, 8 days and 24 days after inoculation. These genes are candidate genes that control nodule development, plant-symbiont interaction or nitrogen fixation and assimilation.

TABLE 3
Soybean TFs regulated by nodulation

| | | | 4DAI inoculated/ | | | 8DAI inoculated/ | | | 24DAI inoculated/ | | |
|---|---|---|---|---|---|---|---|---|---|---|---|
| | | | uninoculated | | | uninoculated | | | uninoculated | | |
| | | | | standard | | | standard | | | standard | |
| Soybean gene ID | ID number | putative function | average | error | T-test | average | error | T-test | average | error | T-test |
| Glyma13g34920 | S4870460 | AP2/EREBP | null | null | null | null | null | null | 0.0041 | 0.0010 | 0.0254 |
| Glyma03g27250 | S4925538 | Zinc finger (GATA) | 2.7610 | 1.2381 | 0.1782 | 1.1661 | 0.3447 | 0.7931 | 0.0930 | 0.0189 | 0.0003 |
| Glyma06g10400 | S15937116 | DNA-binding protein | 0.7604 | 0.1929 | 0.2622 | 0.6342 | 0.0154 | | 0.1056 | 0.0254 | 0.0018 |
| Glyma10g43630 | BI321317 | Zinc finger (C2H2) | 1.1479 | 0.5524 | 0.9952 | 1.5142 | 0.3195 | 0.5968 | 0.1113 | 0.0044 | 0.0397 |
| Glyma15g18580 | S5025536 | Basic Helix-Loop-Helix | 1.7694 | 0.6192 | 0.3160 | 1.0650 | 0.3202 | 0.9332 | 0.1169 | 0.0528 | 0.0150 |
| | | (bHLH) | | | | | | | | | |
| Glyma20g38260 | S5055354 | nucleic acid single- | 0.9342 | 0.2630 | | null | null | null | 0.1261 | 0.0752 | 0.0126 |
| | | stranded binding protein | | | | | | | | | |
| Glyma04g05820 | BE807568 | Trihelix, Triple-Helix | 1.1654 | 0.2850 | 0.8227 | 1.1297 | 0.4484 | 0.7938 | 0.1972 | 0.0985 | 0.0040 |
| | | transcription factor | | | | | | | | | |
| Glyma10g33810 | TC206902 | AP2/EREBP | 1.0222 | 0.1972 | 0.7975 | 1.0252 | 0.2560 | 0.8413 | 0.1980 | 0.0274 | 0.0064 |
| Glyma19g26400 | S4874738 | WRKY | 1.0750 | 0.2885 | 0.8497 | 0.6926 | 0.1175 | 0.2617 | 0.1999 | 0.0688 | 0.0384 |
| Glyma18g29400 | S21537044 | AP2/EREBP | 1.1727 | 0.1290 | 0.3358 | 0.7855 | 0.4581 | 0.6265 | 2.0647 | 0.3327 | 0.0162 |
| Glyma10g42280 | S21537611 | TCP transcription factor | 0.9488 | 0.1247 | 0.5827 | 1.3880 | 0.2083 | 0.1428 | 2.0656 | 0.2021 | 0.0149 |
| Glyma12g36540 | S4935933 | CCAAT-box binding | 1.1503 | 0.1860 | 0.4094 | 1.3646 | 0.1570 | 0.0769 | 2.1097 | 0.3208 | 0.0105 |
| | | trancription factor | | | | | | | | | |
| Glyma12g04050 | TC232817 | Basic Leucine Zipper | 0.9649 | 0.1227 | 0.6352 | 1.3929 | 0.1991 | 0.1372 | 2.1559 | 0.2155 | 0.0167 |
| | | (bZIP) | | | | | | | | | |
| Glyma10g09410 | BI700659 | E2F transcription factor | 1.0123 | 0.0596 | 0.8801 | 1.6292 | 0.4756 | 0.1134 | 2.2668 | 0.5909 | 0.0317 |
| Glyma03g27050 | S23071305 | AP2/EREBP | 1.0683 | 0.1425 | 0.6706 | 1.1717 | 0.1123 | 0.5999 | 2.3737 | 0.4996 | 0.0121 |
| Glyma07g37980 | S5129446 | Zinc finger (C3H) | 1.2206 | 0.1404 | 0.3311 | 1.0549 | 0.0576 | 0.4002 | 2.3915 | 0.4416 | 0.0016 |
| Glyma10g42660 | S4913507 | Zinc finger (C2H2) | 1.0050 | 0.1657 | 0.9669 | 0.9960 | 0.0282 | 0.9611 | 2.7025 | 0.0492 | 0.0001 |
| Glyma13g30750 | TC211634 | ARF | 0.8151 | 0.0087 | 0.2390 | 1.2921 | 0.4398 | 0.5716 | 2.8829 | 0.4239 | 0.0062 |
| Glyma19g32340 | CD409339 | Zinc finger (C3H) | 0.8513 | 0.1819 | 0.2863 | 1.1554 | 0.2271 | 0.4972 | 2.9131 | 0.8257 | 0.0496 |
| Glyma09g37800 | S34818018 | Basic Leucine Zipper | 0.9686 | 0.2486 | 0.7747 | 1.1879 | 0.2154 | 0.6097 | 3.3727 | 1.5487 | 0.0161 |
| | | (bZIP) | | | | | | | | | |
| Glyma08g22190 | S5146871 | AUX/IAA | 0.6252 | 0.1419 | 0.3734 | 1.1201 | 0.5247 | 0.8074 | 3.4143 | 0.5200 | 0.0344 |
| Glyma03g30650 | BU546675 | NAC | 1.2833 | 0.4010 | 0.5563 | 1.2886 | 0.0867 | 0.0371 | 3.7703 | 0.3376 | 0.0428 |
| Glyma19g29670 | BU926469 | MYB | 0.9438 | 0.1614 | 0.7317 | 1.5806 | 0.3393 | 0.1133 | 4.0482 | 0.4318 | 0.0061 |
| Glyma13g41500 | BQ613064 | RNA binding protein | 1.1564 | 0.0456 | 0.3395 | 1.1898 | 0.2049 | 0.4907 | 4.2031 | 0.7354 | 0.0187 |
| Glyma05g22860 | S5132128 | Basic Leucine Zipper | 1.5438 | 0.1840 | 0.0347 | 1.3781 | 0.2110 | 0.0742 | 4.6022 | 0.9991 | 0.0001 |
| | | (bZIP) | | | | | | | | | |
| Glyma19g37410 | S5146199 | Putative trancription | 0.8374 | 0.1658 | 0.4889 | 1.2023 | 0.2185 | 0.5208 | 5.0210 | 0.6797 | 0.0122 |
| | | factor | | | | | | | | | |
| Glyma19g34380 | S5146870 | AUX/IAA | 1.0066 | 0.2793 | 0.9851 | 1.1874 | 0.1551 | 0.5041 | 7.8049 | 2.9402 | 0.0016 |
| Glyma01g24880 | S4983140 | Putative trancription | 0.9389 | 0.3863 | 0.5745 | null | null | null | 151.7420 | 28.6031 | 0.0012 |
| | | factor | | | | | | | | | |
| Glyma18g49360 | S23069986 | MYB | 0.8181 | 0.1675 | 0.3751 | 1.0255 | 0.3074 | 0.9919 | 47.7709 | 18.4422 | 0.0015 |
| Glyma08g15050 | S23065233 | Putative trancription | 1.5286 | 0.5863 | 0.3851 | 1.4524 | 0.6690 | 0.9583 | 0.2158 | 0.0385 | 0.0449 |
| | | factor | | | | | | | | | |
| Glyma10g03820 | S4875903 | WRKY | 1.0083 | 0.1463 | 0.9516 | 0.8669 | 0.0792 | 0.1634 | 0.2209 | 0.0628 | 0.0046 |
| Glyma07g06620 | BU761457 | Basic Leucine Zipper | 0.9533 | 0.2330 | 0.7092 | 1.0947 | 0.3143 | 0.8183 | 0.2393 | 0.1377 | 0.0247 |
| | | (bZIP) | | | | | | | | | |
| Glyma08g47520 | AW185294 | NAC | 0.7773 | 0.1326 | 0.0981 | 1.0578 | 0.3354 | 0.6729 | 0.2409 | 0.1048 | 0.0158 |
| Glyma08g28010 | AW507968 | Basic Helix-Loop-Helix | 0.8930 | 0.1309 | 0.4916 | 1.4171 | 0.3733 | 0.2802 | 0.2426 | 0.1314 | 0.0335 |
| | | (bHLH) | | | | | | | | | |
| Glyma18g04250 | CA936556 | MYB | 1.1707 | 0.2022 | 0.5279 | 1.3043 | 0.2824 | 0.6232 | 0.2429 | 0.0415 | 0.0005 |
| Glyma02g07760 | S21565729 | NAC | 0.9406 | 0.0987 | 0.4297 | 0.9266 | 0.0731 | 0.7875 | 0.2745 | 0.0483 | 0.0075 |
| Glyma16g25250 | BI469606 | MYB | 1.3212 | 0.2494 | 0.2994 | 0.8117 | 0.1320 | 0.3082 | 0.2795 | 0.0472 | 0.0094 |
| Glyma05g29300 | S4918062 | Putative trancription | 1.0378 | 0.2134 | 0.8786 | 1.0915 | 0.1172 | 0.3748 | 0.2829 | 0.0317 | 0.0197 |
| | | factor | | | | | | | | | |
| Glyma02g00870 | S21567471 | AP2/EREBP | 2.4161 | 1.4434 | | 0.6669 | 0.3493 | 0.2401 | 0.2846 | 0.1714 | 0.0398 |
| Glyma06g17330 | S21565817 | Basic Helix-Loop-Helix | 1.1535 | 0.8609 | 0.3088 | 1.1882 | 0.1552 | 0.3092 | 0.2947 | 0.1238 | 0.0490 |
| | | (bHLH) | | | | | | | | | |
| Glyma11g15180 | TC209021 | MYB | 0.7496 | 0.1867 | 0.2943 | 1.1878 | 0.3354 | 0.8175 | 0.2984 | 0.1063 | 0.0227 |
| Glyma17g36370 | CA852521 | MYB | 0.8230 | 0.1616 | 0.2173 | 0.6856 | 0.0771 | 0.2644 | 0.3023 | 0.1798 | 0.0156 |
| Glyma03g38040 | S23068160 | MYB | 1.2749 | 0.1861 | 0.3516 | 1.2714 | 0.4377 | 0.8142 | 0.3097 | 0.0370 | 0.0225 |
| Glyma18g49290 | BE211253 | homeobox | 1.0295 | 0.0622 | 0.8308 | 0.8473 | 0.1265 | 0.3704 | 0.3129 | 0.0747 | 0.0012 |
| Glyma02g39870 | S4911583 | WRKY | 1.1196 | 0.1051 | 0.4969 | 1.0034 | 0.0764 | 0.9774 | 0.3179 | 0.0526 | 0.0101 |
| Glyma17g15330 | S4882412 | MYB | 1.1342 | 0.2042 | 0.5399 | 0.7354 | 0.1591 | 0.2876 | 0.3214 | 0.0159 | 0.0194 |
| Glyma03g29190 | CD403874 | Heat Shock | 0.7127 | 0.2722 | 0.2374 | null | null | null | 0.3249 | 0.1398 | 0.0206 |
| Glyma11g31400 | S15849732 | AP2/EREBP | 1.0140 | 0.3891 | 0.6382 | 1.2984 | 0.2967 | 0.3441 | 0.3253 | 0.0606 | 0.0192 |
| Glyma08g23380 | S5871333; | WRKY | 1.4950 | 0.1788 | 0.0166 | 1.2729 | 0.2751 | 0.6005 | 0.3260 | 0.0995 | 0.0468 |
| | TC225723 | | | | | | | | | | |
| Glyma13g39990 | S4937572 | Putative trancription | null | null | null | 0.0739 | 0.0515 | 0.1236 | 0.3281 | 0.1650 | 0.0329 |
| | | factor | | | | | | | | | |
| Glyma04g39650 | TC221320 | WRKY | 1.1538 | 0.4635 | 0.7449 | 1.1197 | 0.2534 | 0.9211 | 0.3330 | 0.1177 | 0.0114 |
| Glyma13g26790 | S15850286 | MYB | 1.3668 | 0.6214 | 0.9855 | 1.2882 | 0.6793 | 0.8892 | 0.3378 | 0.1162 | 0.0352 |
| Glyma15g42380 | S5874971 | homeobox | 0.8199 | 0.1138 | 0.1728 | 0.9709 | 0.0327 | 0.9446 | 0.3396 | 0.0718 | 0.0297 |
| Glyma03g42450 | BI468894 | ERF | 1.3218 | 0.3525 | 0.3497 | 1.1025 | 0.3557 | 0.8416 | 0.3409 | 0.1496 | 0.0460 |
| Glyma08g05240 | TC210810 | Telomeric DNA binding | 0.8258 | 0.0486 | 0.1032 | 1.0829 | 0.0788 | 0.7860 | 0.3453 | 0.0743 | 0.0224 |
| | | protein | | | | | | | | | |
| Glyma01g02210 | S21700413 | Putative trancription | 0.7219 | 0.1185 | 0.1956 | 1.0696 | 0.1139 | 0.6855 | 0.3462 | 0.0749 | 0.0063 |
| | | factor | | | | | | | | | |
| Glyma15g12930 | BM955055 | MYB | 1.2772 | 0.1592 | 0.2876 | 1.6597 | 0.8282 | 0.6742 | 0.3476 | 0.0789 | 0.0072 |
| Glyma13g03700 | S5035170 | EIL transcription factor | 1.0633 | 0.2527 | 0.9572 | 1.0433 | 0.2308 | 0.9362 | 0.3530 | 0.0693 | 0.0285 |
| Glyma18g51680 | TC222644 | AP2/EREBP | 1.0475 | 0.2480 | 0.8205 | 0.8431 | 0.2389 | 0.4574 | 0.3611 | 0.0843 | 0.0060 |
| Glyma20g07050 | S21566080 | Zinc finger (Constans) | 0.8561 | 0.1378 | 0.1995 | 0.9250 | 0.0635 | 0.7803 | 0.3683 | 0.0749 | 0.0438 |
| Glyma07g37000 | S5088770 | Putative trancription | 0.8949 | 0.1126 | 0.5691 | 1.0733 | 0.1454 | 0.7785 | 0.3802 | 0.0074 | 0.0012 |
| | | factor | | | | | | | | | |
| Glyma08g10550 | BE440918 | ARF | 1.0060 | 0.1462 | 0.9541 | 1.1239 | 0.1115 | 0.7283 | 0.3820 | 0.0990 | 0.0023 |
| Glyma13g01930 | TC215663 | AP2/EREBP | 0.7809 | 0.1389 | 0.1295 | 0.8062 | 0.0327 | 0.0750 | 0.3855 | 0.0877 | 0.0173 |
| Glyma20g26700 | BE347092 | homeobox | 1.1685 | 0.2355 | 0.7085 | 0.8903 | 0.1832 | 0.3591 | 0.3883 | 0.1272 | 0.0083 |
| Glyma11g14040 | TC205929 | AP2/EREBP | 1.0685 | 0.1079 | 0.9306 | 2.0874 | 0.5212 | 0.0513 | 0.3886 | 0.0443 | 0.0173 |
| Glyma13g40830 | S34273475 | MYB | 0.9417 | 0.1920 | 0.5502 | 0.8718 | 0.1023 | 0.3014 | 0.3895 | 0.1084 | 0.0062 |
| Glyma03g41750 | TC209320 | WRKY | 1.4823 | 0.5589 | 0.3749 | 1.6455 | 0.7602 | 0.5298 | 0.3943 | 0.1082 | 0.0108 |
| Glyma04g06620 | CA800598 | CCR4-NOT transcription | 0.9729 | 0.0484 | 0.8915 | 0.8324 | 0.0885 | 0.1191 | 0.4053 | 0.1565 | 0.0203 |
| | | factor protein | | | | | | | | | |
| Glyma16g02570 | S23062212 | MYB | 1.2342 | 0.2333 | 0.3848 | 0.9812 | 0.1631 | 0.7190 | 0.4099 | 0.0342 | 0.0123 |
| Glyma08g02930 | S5103646 | MADS-box transcription | 1.0981 | 0.2118 | 0.6936 | 0.8036 | 0.0353 | 0.0996 | 0.4124 | 0.0754 | 0.0166 |
| | | factor | | | | | | | | | |
| Glyma01g00980 | CF808484 | RNA polymerase | 1.1548 | 0.1079 | 0.3052 | 1.3258 | 0.2230 | 0.4004 | 0.4311 | 0.0623 | 0.0111 |
| Glyma06g07110 | S21539760 | RNA binding protein | 1.0194 | 0.0779 | 0.8477 | 0.9515 | 0.0679 | 0.7690 | 0.4333 | 0.0839 | 0.0088 |
| Glyma08g09970 | S4916522 | Zinc finger (C2H2) | 1.2207 | 0.2167 | 0.4408 | 0.9998 | 0.1144 | 0.9344 | 0.4387 | 0.0580 | 0.0014 |
| Glyma08g40840 | S23072300 | Zinc finger transcription | 0.7525 | 0.1954 | 0.1852 | 1.0100 | 0.2741 | 0.8588 | 0.4388 | 0.1056 | 0.0298 |
| | | factor | | | | | | | | | |
| Glyma18g04060 | S21567638 | DNA-binding protein | 0.8622 | 0.2695 | 0.3083 | 1.0005 | 0.1033 | 0.9705 | 0.4392 | 0.0554 | 0.0262 |
| Glyma04g04170 | TC229348 | Basic Leucine Zipper | 0.9751 | 0.1371 | 0.6604 | 0.9994 | 0.1136 | 0.8394 | 0.4426 | 0.0699 | 0.0296 |
| | | (bZIP) | | | | | | | | | |
| Glyma16g34490 | BE058375 | MYB | 1.0663 | 0.0958 | 0.6121 | 0.8559 | 0.0752 | 0.0655 | 0.4456 | 0.0708 | 0.0032 |
| Glyma04g43350 | S23069218 | ARF | 0.9859 | 0.0722 | 0.8526 | 0.9822 | 0.0488 | 0.6723 | 0.4498 | 0.0395 | 0.0425 |
| Glyma02g47640 | S23062201 | GRAS | 1.3510 | 0.0920 | 0.0816 | 0.8958 | 0.0701 | 0.2491 | 0.4506 | 0.0475 | 0.0093 |
| Glyma18g00840 | CA802838 | calmodulin binding/ | 0.8793 | 0.1428 | 0.4504 | 0.9442 | 0.1922 | 0.4961 | 0.4512 | 0.0579 | 0.0157 |
| | | transcription regulator | | | | | | | | | |
| Glyma04g38730 | S4991641 | SRT2 DNA binding | 0.9981 | 0.0984 | 0.9424 | 0.9012 | 0.0941 | 0.2597 | 0.4583 | 0.1385 | 0.0276 |
| | | protein | | | | | | | | | |
| Glyma16g01500 | S16535713 | AP2/EREBP | 0.8188 | 0.1319 | 0.1801 | 1.0489 | 0.1163 | 0.8918 | 0.4610 | 0.0945 | 0.0495 |
| Glyma02g38870 | CF806129 | Zinc finger (Constans) | 0.8538 | 0.0911 | 0.1033 | 0.9632 | 0.2704 | 0.5319 | 0.4611 | 0.1052 | 0.0335 |
| Glyma13g38630 | S5052631 | WRKY | 0.4547 | 0.2339 | 0.2011 | 0.8259 | 0.0097 | 0.2332 | 0.4629 | 0.0997 | 0.0258 |
| Glyma13g36540 | S4873428 | WRKY | 1.0814 | 0.2457 | 0.8593 | 0.9587 | 0.0670 | 0.6690 | 0.4651 | 0.0393 | 0.0354 |
| Glyma06g45770 | TC208469 | BTB-POZ domain | 0.8203 | 0.1084 | 0.1372 | 0.9540 | 0.1041 | 0.5595 | 0.4662 | 0.0308 | 0.0104 |
| | | containing protein | | | | | | | | | |
| Glyma03g33900 | S4916150 | SWI2/SNF2 | 1.0370 | 0.2073 | 0.9081 | 1.2713 | 0.2168 | 0.2528 | 0.4741 | 0.0885 | 0.0209 |
| Glyma17g16930 | S4898544 | homeobox | 1.0337 | 0.1258 | 0.8089 | 0.8724 | 0.1388 | 0.3013 | 0.4763 | 0.0294 | 0.0003 |
| Glyma06g11010 | S23065007; | AP2/EREBP | 1.1101 | 0.1506 | 0.4878 | 0.9704 | 0.0980 | 0.9202 | 0.4781 | 0.0688 | 0.0212 |
| | TC225047 | | | | | | | | | | |
| Glyma14g17730 | S22953012 | WRKY | 1.3342 | 0.2613 | 0.1882 | 1.0379 | 0.0247 | 0.6640 | 0.4783 | 0.0468 | 0.0317 |
| Glyma01g40380 | S5142323 | AP2/EREBP | 0.8435 | 0.1130 | 0.1451 | 1.0290 | 0.0598 | 0.8371 | 0.4816 | 0.0562 | 0.0048 |
| Glyma06g01300 | S21537813 | Putative trancription | 0.8343 | 0.1654 | 0.2005 | 1.1674 | 0.0547 | 0.1321 | 0.4878 | 0.0601 | 0.0046 |
| | | factor | | | | | | | | | |
| Glyma09g03690 | S21538601 | MYB | 1.3245 | 0.2860 | 0.3070 | 1.0924 | 0.4345 | 0.7433 | 0.4922 | 0.1123 | 0.0185 |
| Glyma20g30650 | BI945044 | GT2 transcription factor | 0.9957 | 0.1774 | 0.8315 | 0.8892 | 0.0798 | 0.5330 | 0.4929 | 0.1354 | 0.0156 |
| Glyma14g24290 | S5030305 | SWIRM | 1.2861 | 0.1341 | 0.3337 | 0.8821 | 0.0535 | 0.6059 | 0.4992 | 0.0346 | 0.0482 |
| Glyma13g05270 | S5115730 | homeobox | 0.8988 | 0.0397 | 0.3734 | 1.2276 | 0.1554 | 0.3701 | 0.4210 | 0.1351 | 0.0463 |
| Glyma17g15480 | CD392418 | AP2/EREBP | 0.9608 | 0.4122 | 0.8250 | 0.7739 | 0.0666 | 0.7026 | 0.4330 | 0.2568 | 0.0422 |
| Glyma05g20460 | TC210199 | Heat Shock | 1.2608 | 0.2567 | 0.4055 | 0.9835 | 0.1699 | 0.7049 | 0.4697 | 0.0216 | 0.0038 |
| Glyma03g38360 | TC212079 | WRKY | 0.9683 | 0.0588 | 0.7941 | 0.8400 | 0.1458 | 0.2406 | 0.4713 | 0.0491 | 0.0237 |
| Glyma07g16170 | BG790017 | ARF | 0.9410 | 0.0803 | 0.6827 | 1.0808 | 0.2229 | 0.9300 | 0.4976 | 0.0693 | 0.0452 |
| Glyma06g21020 | S5146166 | NAC | 1.1051 | 0.1515 | 0.8157 | 0.7941 | 0.1055 | 0.2808 | 0.4231 | 0.0543 | 0.0042 |
| Glyma19g31940 | S21566681 | Heat Shock | 0.9619 | 0.5212 | 0.7035 | 0.7648 | 0.3109 | 0.2292 | 0.2116 | 0.0222 | 0.0053 |
| Glyma02g15920 | TC207514 | WRKY | 0.8653 | 0.0569 | 0.1970 | 0.9529 | 0.0585 | 0.7881 | 0.2216 | 0.0500 | 0.0158 |
| Glyma08g41620 | CD398155 | Basic Helix-Loop-Helix | 0.8224 | 0.0664 | 0.4187 | 0.9041 | 0.1365 | 0.5857 | 0.3323 | 0.0900 | 0.0015 |
| | | (bHLH) | | | | | | | | | |
| Glyma13g29600 | TC222844 | WRKY | 1.2688 | 0.3646 | 0.5880 | 1.1817 | 0.0802 | 0.3056 | 0.3511 | 0.0337 | 0.0014 |
| Glyma05g28960 | TC216155 | Basic Leucine Zipper | 0.9342 | 0.1680 | 0.4743 | 0.9865 | 0.3481 | 0.8462 | 2.7218 | 0.7822 | 0.0190 |
| | | (bZIP) | | | | | | | | | |
| Glyma02g42200 | S5142660 | homeobox | 1.8122 | 0.2169 | 0.0538 | 2.6317 | 1.0563 | 0.0328 | 0.3776 | 0.2415 | |
| Glyma01g02760 | S5096279 | AP2/EREBP | 1.3732 | 0.2569 | 0.2281 | 2.6576 | 0.9045 | 0.0438 | 0.7916 | 0.0852 | 0.4686 |
| Glyma07g14610 | BG650304 | SBP (squamosa) | 0.6999 | 0.1691 | 0.1354 | 6.7245 | 1.8803 | 0.0023 | 0.6831 | 0.0664 | |
| Glyma06g08610 | S21566814 | DNA methyltransferase | 0.9672 | 0.1052 | 0.6099 | 2.6527 | 0.2000 | 0.0058 | 1.3852 | 0.2100 | 0.1410 |
| | | MET | | | | | | | | | |
| Glyma09g33240 | TC234528 | AP2/EREBP | 1.2172 | 0.1224 | 0.3082 | 4.2588 | 1.9736 | 0.0370 | 1.4063 | 0.6678 | 0.7125 |
| Glyma14g03100 | AW433203; | MADS-box transcription | 0.5703 | 0.2149 | | 0.2785 | 0.0103 | 0.0428 | 121.5298 | 82.1908 | 0.4000 |
| | S4907367 | factor | | | | | | | | | |
| Glyma03g27180 | S6675747 | SBP (squamosa) | 0.8921 | 0.2391 | 0.7628 | 4.1947 | 1.4340 | 0.0078 | 0.7373 | 0.4142 | |
| Glyma03g26700 | AI795005 | homeobox | 1.2921 | 0.2658 | 0.3942 | 2.6577 | 0.5534 | 0.0074 | null | null | null |
| Glyma08g01720 | S4932151; | DNA-binding protein | 0.9799 | 0.1063 | 0.7141 | 2.0629 | 0.3361 | 0.0048 | 1.5672 | 0.7780 | 0.7498 |
| | S4932199 | | | | | | | | | | |
| Glyma03g31980 | S23065855 | MYB | 0.7106 | 0.1967 | | 4.2979 | 1.4269 | 0.0463 | 5.6824 | 3.1100 | 0.0649 |
| Glyma05g38580 | BU549908 | Gt-2 related transcription | 1.4156 | 0.1620 | 0.1199 | 6.4978 | 1.5640 | 0.0025 | 3.1237 | 1.5513 | |
| | | factor | | | | | | | | | |
| Glyma03g42260 | S34273417 | MYB | 0.3535 | 0.0639 | 0.0182 | 0.5732 | 0.2556 | 0.1130 | 0.0562 | 0.0169 | 0.1460 |
| Glyma12g34510 | AW831868 | CCAAT-box binding | 17.3134 | 3.5968 | 0.0003 | 4.9513 | 1.2052 | 0.0253 | 0.5121 | 0.2223 | 0.0483 |
| | | trancription factor | | | | | | | | | |
| Glyma02g35190 | S4925563 | CCAAT-box binding | 2.5915 | 0.5040 | 0.0051 | 3.3677 | 0.8492 | 0.0351 | 2.4274 | 0.7438 | 0.0713 |
| | | trancription factor | | | | | | | | | |
| Glyma16g04410 | BI971027 | AP2/EREBP | 2.6167 | 0.1800 | 0.0008 | 3.0160 | 0.7454 | 0.0064 | 1.3674 | 0.5438 | 0.5911 |
| Glyma17g07330 | S23061916 | MYB | 0.9442 | 0.0613 | 0.4210 | 2.1859 | 0.2877 | 0.0013 | 5.7650 | 1.0579 | 0.0002 |
| Glyma16g26290 | S22951832 | Basic Helix-Loop-Helix | 1.0193 | 0.0470 | 0.9066 | 2.9187 | 0.3793 | 0.0006 | 7.4517 | 1.6829 | 0.0001 |
| | | (bHLH) | | | | | | | | | |
| Glyma13g40240 | AW568213 | Zinc finger (C2H2) | 0.8720 | 0.1869 | 0.6470 | 4.9161 | 0.6953 | 0.0096 | 7.8311 | 1.4691 | 0.0008 |
| Glyma01g01210 | S21537528 | RNA-dependent RNA | 1.1556 | 0.2210 | 0.5509 | 2.1941 | 0.2437 | 0.0087 | 4.2572 | 0.9753 | 0.0486 |
| | | polymerase | | | | | | | | | |
| Glyma10g10240 | S5108906 | CCAAT-box binding | 6.8243 | 0.9302 | 0.0214 | 13.7461 | 3.8739 | 0.0007 | 6.8275 | 1.8162 | 0.0250 |
| | | trancription factor | | | | | | | | | |

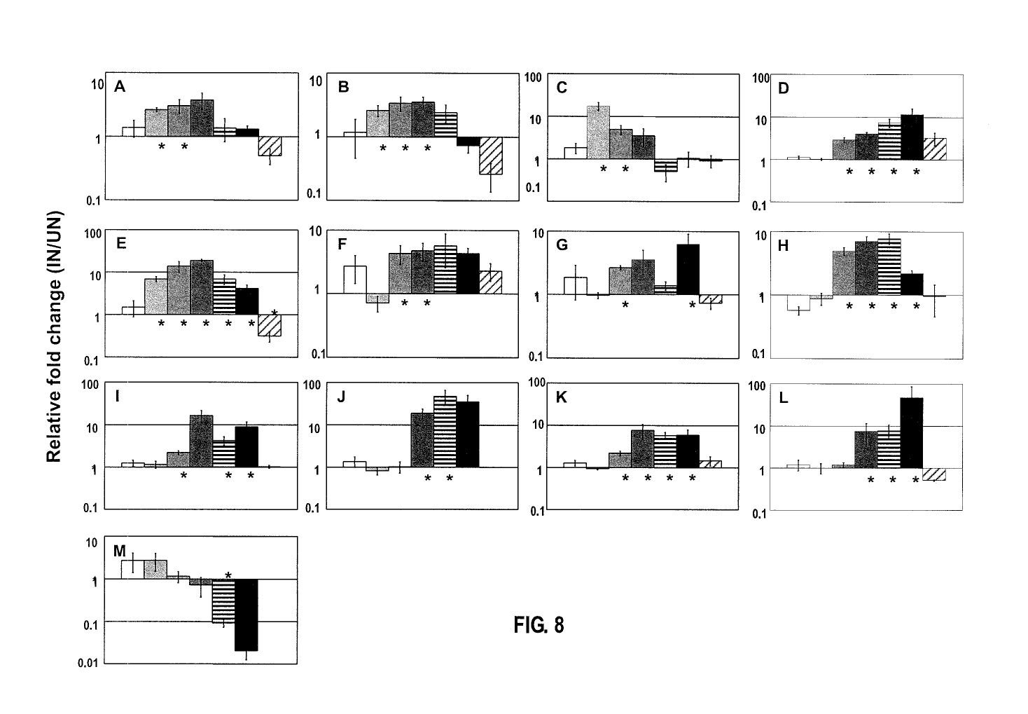

The expression pattern of 13 of these TF genes through different stages of nodule development after inoculation of B. japonicum are shown in FIG. 8. These 13 genes are: panel A: Glyma16g04410 (AP2/EREBP); B: Glyma02g35190 (CCAAT-Box); C: Glyma12g34510 (CCAAT-Box); D: Glyma16g26290 (bHLH); E: Glyma10g10240 (putative transcription factor); F: Glyma03g31980 (Myb); G: Glyma06g08610 (DNA methyltransferase); H: Glyma13g40240 (Zinc Finger); I: Glyma01g01210 (RNA-dependent RNA polymerase); J: Glyma18g49360 (Myb); K: Glyma17g07330 (Myb); L: Glyma19g34380 (Aux/IAA); M: Glyma03g27250 (Zinc finger (GATA). The expression pattern through different stages of nodule development 0 (white bar), 4 (light grey bars), 8 (grey bars), 16 (dark grey bars), 24 (black grey bars) and 32 days (black bars) after B. japonicum inoculation and in response to KNO₃ treatment (open bars) are shown. “*” means the data were statistically significant.

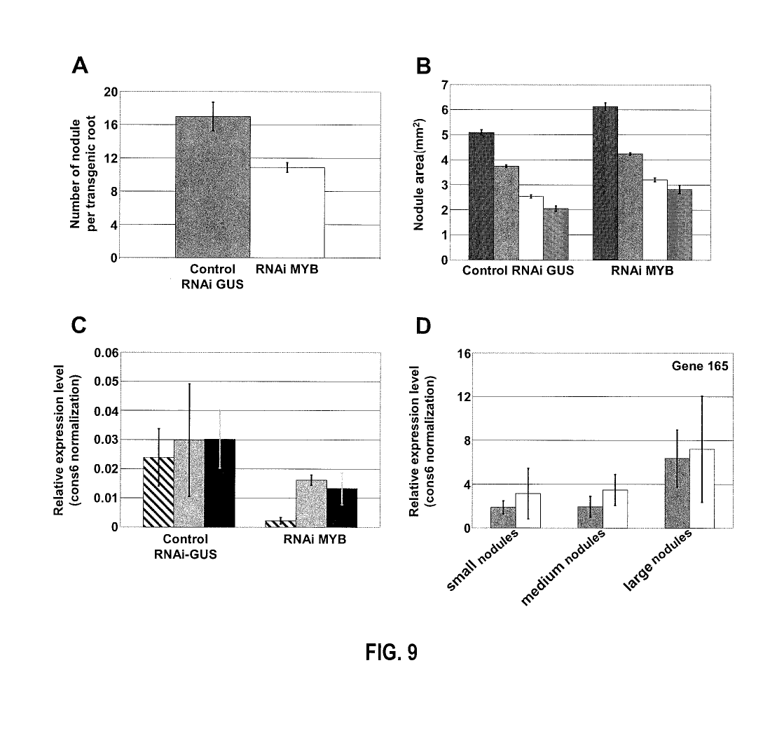

Using a RNAi gene-silencing strategy, the functions of some TFs implicated in nodule development were further characterized. When one of these TFs, MYB, was silenced, lower number but bigger nodules were observed. This result suggests that this MYB gene plays a role in the nodulation process (FIG. 9).

Panel A of FIG. 9 compares the number of nodules between RNAi-GUS (grey bar) and RNAi 523065855 soybean roots (white bar). The number of nodules was reduced when expression of the 523065855 gene was suppressed. Panel B shows the comparison of nodule size between RNAi-GUS (left) and RNAi 523065855 (right) roots. According to their size, nodules were divided in four categories: large (dotted bars), medium (grey bars) and small nodules with leghemoglobin (white bars) and immature nodules (i.e. lack of leghemoglobin; vertical striped bars). Panel C shows gene expression levels of 523065855 in RNAi-GUS (left) and RNAi 523065855 (right) nodules to confirm that the RNA silencing worked. Transcriptomic analysis was performed on large, medium and small size nodule (open, grey and black bars respectively). Gene expression levels were normalized using Cons6 gene. Panel D shows the expression levels of a gene, Glyma19g34740, which shares strong nucleotide sequences homology with, but is different from 523065855. The expression levels of Glyma19g34740 were not altered by RNAi 523065855, indicating the specificity of RNAi construct in the silencing of 523065855. Gene expression levels were quantified by qRT-PCR on RNAi-GUS (grey bars) and RNAi 523065855 (white bars) small, medium and large nodules and were normalized by Cons6 gene.

Next, the localization of the TF genes during nodulation was determined by using the GUS or GFP reporter genes system described above. Transcriptional fusions containing promoter sequences of the TF genes and coding sequence of the reporter gene were constructed and introduced into soybean plants. Briefly, Gateway system (Invitrogen, Carlsbad, Calif.) was used to clone the promoter of the Glyma03g31980 gene upstream of the GFP and GUS cDNAs. By mining genomic sequences available on Phytozome website (http://www.phytozome.net/soybean.php), a 1967 by DNA fragment 5′ to the first codon of the Glyma03g31980 gene was identified. By two independent PCR reactions, the AttB sites were created at the extremities of the promoter sequences. Soybean Williams 82 genomic DNA was used as template and the following primers were used for these two PCRs:

First PCR:
Glyma03g31980promoAttB-for:
5′-AAAAAGCAGGCTCCTACATGAATATGTGTTCAAAATA
and
Glyma03g31980promoAttB-rev:
5′-AGAAAGCTGGGTTTTGATGACTTAGACTACTCCTTC
Second PCR:
universal AttB primers-attB1 adaptor:
5′-GGGGACAAGTTTGTACAAAAAAGCAGGCT
and
attB2adaptor:
5′-GGGGACCACTTTGTACAAGAAAGCTGGGT.

Using the Gateway® BP Clonase® II enzyme mix, the Glyma03g31980 promoter fragment was introduced first into the pDONR-Zeo vector (Invitrogen, Carlsbad, Calif.), then into pYXT1 or pYXT2 destination vectors using the Gateway® LR Clonase® II enzyme mix (Invitrogen, Carlsbad, Calif.). pYXT1 or pYXT2 destination vectors carry the GUS or GFP reporter genes, respectively (Xiao et al., 2005). A. rhizogenes (strain K599) was transformed by electroporation with Glyma03g31980promoter-pYXT1 and Glyma03g31980promoter-pYXT2 vectors.

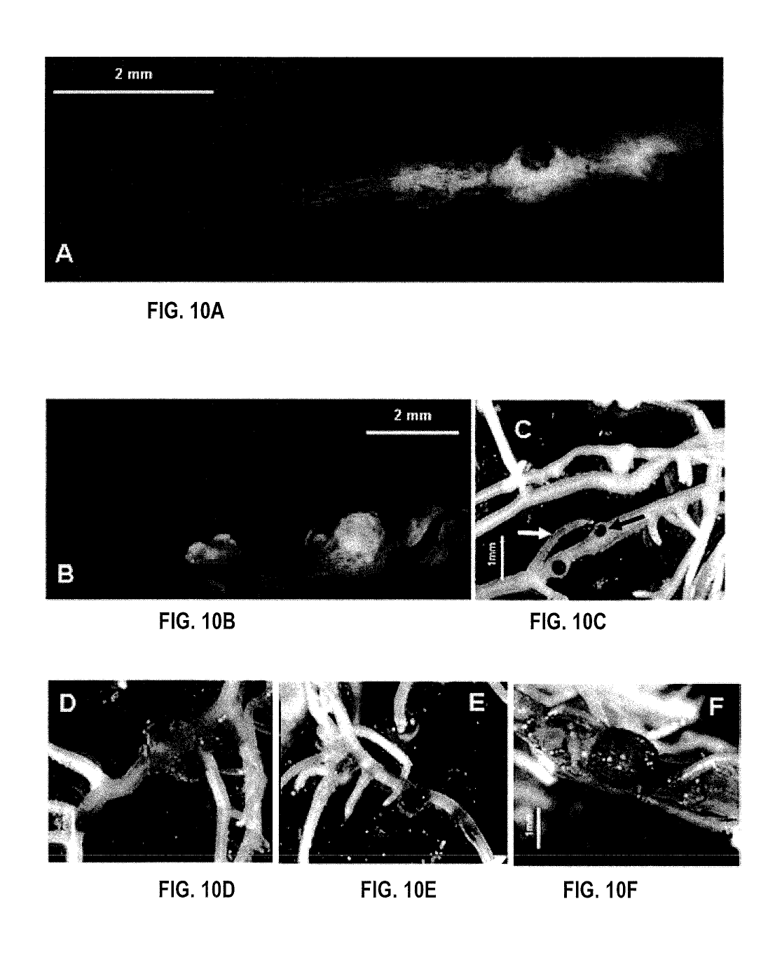

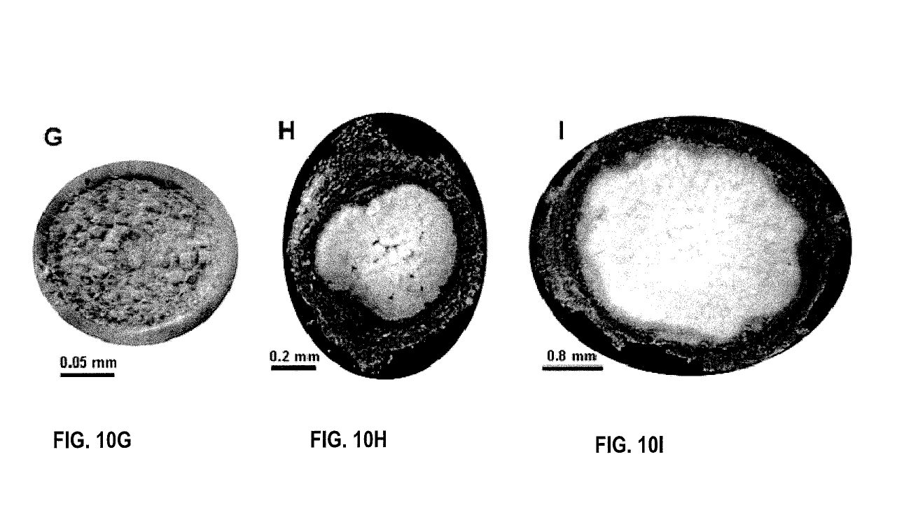

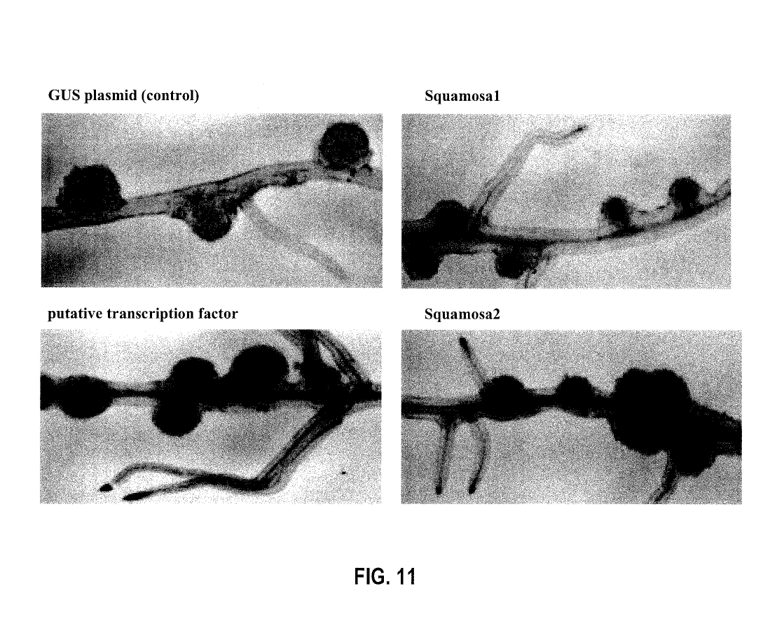

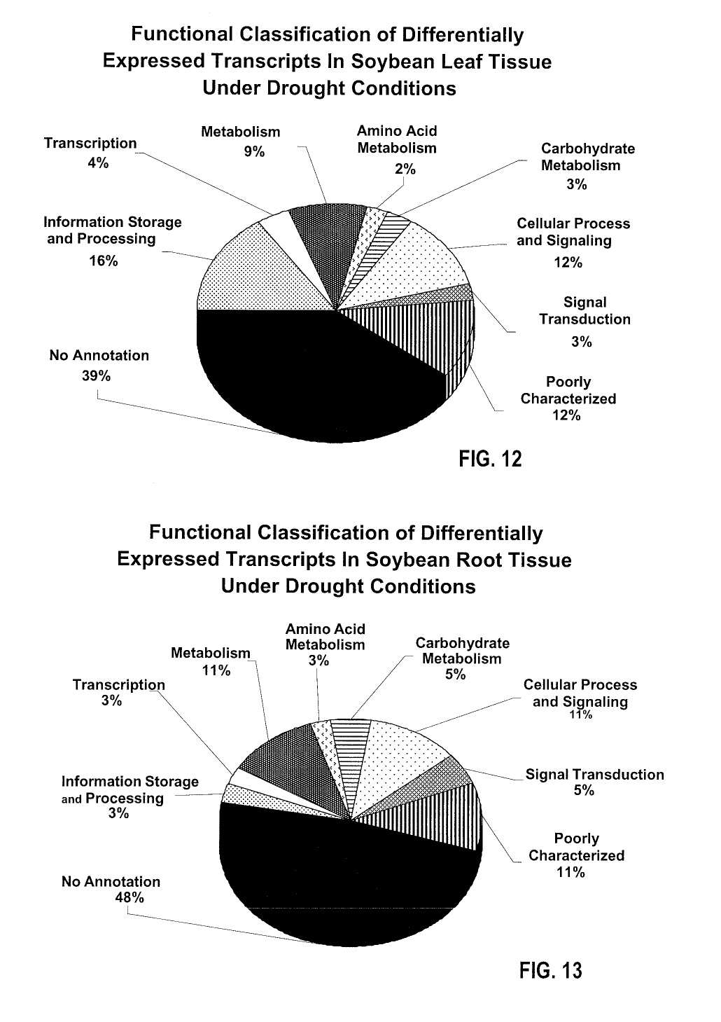

The expression of the reporter genes was monitored by following the GUS (blue) or GFP (green) signals. FIG. 10 shows the expression pattern of a MYB transcription factor during nodulation using GFP (A, B) and GUS (C, D, E, F) as reporter genes, respectively. Sections of root and nodules showed a strong expression of the MYB gene in the epidermal and endodermal cells, and vascular tissues and, in less strong in infected zone of the nodule (G, H, I). Also, as shown in FIG. 10, the MYB TV gene was not exclusively expressed in the nodule (FIG. 10). Expression patterns or other TFs are shown in FIG. 11, which also confirms their strong expression in the soybean nodules. Squamosa1=Glyma07g14610; Squamosa2=Glyma03g27180; Putative Transcription factor=Glyma01g40230.

### Example 7

### Gene Profiling of Drought Response Genes in Soybean

Genetic material and the growing system: cv Williams 82 was used for the green house experiments. Plants were grown in Turface-sand medium in 3 gallon pots. One-month old soybean plants were subjected to gradual stress by withholding water and the samples were collected in three biological replicates. To quantitate the stress level we monitored relative water content (RWC), leaf water potential, and turface-soil mixture water potential and moisture content. Leaf RWC, leaf water potential, and soil water content were 95%.-0.3 MPa, and 20% (v/v), respectively, for well-watered samples. These values were 65%, −1.6 MPa, 9.6% for the water-stressed samples.

RNA isolation and the microarray: Flash-frozen plant tissue samples were ground under liquid nitrogen with a mortar and pestle. Total RNA is extracted using a modified Trizol (Invitrogen Corp., Carlsbad, Calif.) protocol followed by additional purification using RNEasy columns (Qiagen, Valencia, Calif.). RNA quality is assayed using an Agilent 2100Bioanalyzer to determine integrity and purity; RNA purity is further assayed by measuring absorbance at 200 nm and 280 nm using a Nanoprop spectrophotometer.

Microarray hybridization, data acquisition, and image processing: We used the pair wise comparison experimental plan for the microarray experiments. A total number of 12 hybridizations were conducted as: 2 biological conditions×3 biological replicates×2 tissue types. First strand GDNA were synthesized with 30 pg total RNA and T7-Oligo(dT) primer. The total RNA were processed to use on Affymetrix Soybean GeneChip arrays, according to the manufacturer's protocol (Affymetrix, Santa Clara, Calif.). The GeneChip soybean genome array consists of 35,611 soybean transcripts (details as in the results description). Microarray hybridization, washing and scanning with Affymetrix high density scanner were performed according to the standard protocols. The scanned images were processed and the data acquired using GCOS. Having selected genes that are significantly correlated with phenotype or treatment, data mining is conducted using a variety of tools focusing on class discovery and class comparison in order to identify and prioritize candidates.

Confirmation of gene expression by qRT-PCR: Validation of the microarray profiling and the expression of significant genes at significant time points in the experiments were determined by a high-throughput two-step quantitative RT-PCR (qRT-PCR) assay using SYBR Green on the ABI 7900 HT and by the delta delta CT method (Applied Biosystems) developed in course of these studies.

One-month old soybean plants were subjected to gradual stress by withholding water and the samples were collected in three biological replicates. To quantitate the stress level we monitored relative water content (RWC), leaf water potential, and surface-soil mixture water potential and moisture content. Total RNA isolation and microarray hybridizations were conducted using standard protocols. We used 60K soybean Affymetrix GeneChips for the transcriptome profiling. The GeneChip® Soybean Genome Array is a 49-format, 11-micron array design, and it contains 11 probe pairs per probe set. Sequence Information for this array includes public content from GenBank® and dbEST. Sequence clusters were created from UniGene Build 13 (Nov. 5, 2003). The GeneChip® Soybean Genome Array contains ˜60,000 transcripts and 37,500 transcripts are specific for soybean. In addition to extensive soybean coverage, the GeneChip® Soybean Genome Array includes probe sets to detect approximately 15,800 transcripts for Phytophthora sojae (a water mold that commonly attacks soybean crops) as well as 7,500 Heterodera glycines (cyst nematode pathogen) transcripts. (www.affymetrix.com) The affymetrix chip hybridization data of the soybean root under stress were processed. The statistical analysis of the data was performed using the mixed linear model ANOVA (log2 (pm)˜probe+trt+array (trt)). The response variable “log2 (pm)” is the log base 2 transformed perfect match intensity after RMA background correction and quantile normalization; the covarlate “probe” indicates the probe levels since for each gene there are usually 11 probes; “trt” is the treatment/condition effect and it specifies if the array considered is treatment or control; “array(trt)” is the array nested within trt effect, as there are replicate arrays for each treatment.

FDR adjusted p-value is less than 0.01 cutoff point where fdrp is less than 0.01.

The statistically analyzed data were sorted and the functional classifications (KOG and G0) were performed. Significantly differentially expressed transcripts in root and leaf tissues between well-watered and water stressed condition are:

p value adjusted FDR 5%
Leaf tissue—2497 up regulated, 938 down regulated
Root tissue—885 up regulated, 5428 down regulated
Leaf vs root—769 up regulated, 406 down regulated
   p value adjusted FDR 1%
Leaf tissue—2088 up regulated, 863 down regulated
Root tissue—800 up regulated, 5428 down regulated
Leaf vs root—576 up regulated, 211 down regulated

The functional classification of the differentially expressed genes in soybean leaf under drought condition is summarized in Table 4, which shows the numbers of genes that are either up- or down-regulated in each category as defined by protein function.

TABLE 4
Functional Classification of drought responsive transcripts in
soybean leaf tissues:

| | Up | Down | Up + Down |
|---|---|---|---|
| Leaf tissue | regulated | regulated | regulated |
| Information Storage and | 508 | 29 | 537 |
| Processing | | | |
| Transcription | 106 | 27 | 133 |
| Metabolism | 225 | 88 | 313 |
| Amino Acid Metabolism | 74 | 10 | 84 |
| Carbohydrate Metabolism | 80 | 28 | 108 |
| Cellular Process and Signaling | 320 | 80 | 400 |
| Signal Transduction | 42 | 46 | 88 |
| Poorly Characterized | 302 | 102 | 404 |
| No Annotation | 840 | 524 | 1364 |
| Total | 2497 | 934 | 3431 |

Sequences for the genes and proteins disclosed in this disclosure can be found in GenBank, a nucleotide and protein sequence database maintained by the National Center for Biotechnology Information (NCBI), or in the Soybean genome database maintained by the University of Missouri at Columbia, Mo. Both databases are freely available to the general public.

The functional classification of the differentially expressed genes in soybean root under drought condition is summarized in Table 5, which shows the numbers of genes that are either up- or down-regulated in each category as defined by protein function.

TABLE 5
Functional Classification of drought responsive transcripts in
soybean root tissues:

| | Up | Down | Up + Down |
|---|---|---|---|
| Root tissue | regulated | regulated | regulated |
| Information Storage and | 14 | 187 | 201 |
| Processing | | | |
| Transcription | 23 | 147 | 170 |
| Metabolism | 96 | 619 | 715 |
| Amino Acid Metabolism | 28 | 132 | 160 |
| Carbohydrate Metabolism | 36 | 273 | 309 |
| Cellular Process and Signaling | 125 | 599 | 724 |
| Signal Transduction | 44 | 274 | 318 |
| Poorly Characterized | 109 | 574 | 683 |
| No Annotation | 409 | 2624 | 3033 |
| Total | 884 | 5429 | 6313 |

### Example 8

### Identification of Transcription Factors that are Upregulated in Response to Drought Condition

Based on database mining of transcription factors, domain homology analysis, and the soybean microarray data obtained in Example 1 using drought-treated root tissues from greenhouse-grown plants, 199 candidate transcription factor genes or ESTs derived from these genes with putative function for drought tolerance were identified. 64 of the candidates showed high sequence similarity to known transcription factor domains and might possess high potential for drought tolerant gene identification. The remaining 135 of the candidates showed relatively low sequence similarity to known transcription factors domains and thus might represent a valuable resource for the identification of novel genes of drought tolerance. The candidates generally belonged to the NAM, zinc finger, bHLH, MYB, AP2, CCAAT-binding, bZIP and WRKY families.

On the basis of family novelty and the magnitude of drought-inducibility, three transcripts were chosen for a pilot experiment to characterize and isolate promoters for drought tolerance studies. The three candidates were BG156308, BI970909, and BI893889, which belonged to the bHLH, CCAAT-binding, and NAM families, respectively. Under drought condition, the expression levels of these three genes were increased from 2.5 to 252-fold. Moreover, no transcription factor from those families has been reported to control drought tolerance in soybean and other crops. Therefore, these candidate genes may represent novel members of these families that may also play a role in plant drought response. Functional characterization of these transcription factors may help elucidate pathways that are involved in plant drought response.

### Example 9

### Validation of Genes that are Upregulated in Response to Drought Conditions

A set of 62 candidate drought response genes (or DRGs) identified in the microarray experiment were further confirmed by quantitative reverse transcription-PCR (qRT-RCR). Briefly, RNA samples from root or leaf tissues obtained from soybean plants grown under normal or drought conditions were prepared as described in Example 1. cDNA were prepared from these RNA samples by reverse transcription. The cDNA samples thus obtained were then used as template for PCR using primer pairs specific for 64 candidate genes. The PCR products of each gene under either drought or normal conditions were quantified and the results are summarized in Table 6. The Column with the heading “qRT-PCR Root log ratio of expression level” shows the base 2 logarithm of the ratio between the root expression level of the particular gene under drought condition and the expression level of the same gene under normal condition. Similarly, the Column with the heading “qRT-PCR Leaf log ratio of expression level” shows a similar set of data obtained from leaf tissues. The qRT-PCR results are generally consistent with the microarray data, suggesting that the genes whose expression levels are up-regulated or down-regulated are likely to be true Drought Response Genes (DRGs).

TABLE 6
List of the 62 Root Drought Response Genes and the fold change
in their expression levels under drought condition

| | | | qRT-PCR | qRT-PCR |
|---|---|---|---|---|
| | NCBI | | Root log | Leaf log |
| | Accession# | Fold | ratio of | ratio of |
| Item | of soybean | Change in | expression | expression |
| No. | EST | Microarray | level | level |
| 1 | AW100172 | 3.084026621 | 1.1797147 | 0.89568458 |
| 2 | BI700189 | 5.250749017 | 2.89530165 | 0.90051965 |
| 3 | AW101461 | 2.131337965 | 3.21871313 | 1.09980849 |
| 4 | BI701724 | 2.445271745 | 0.77306449 | 2.11599468 |
| 5 | CD405935 | 2.378775421 | 1.76596939 | 0.43572003 |
| 6 | CF806221 | 5.844540021 | 2.70717347 | 1.78868292 |
| 7 | CF806953 | 3.07486286 | 2.42832356 | 31.9623187 |
| 8 | CF807326 | 2.533554706 | 4.31347621 | 0.86931523 |
| 9 | CF807343 | 8.420142043 | 2.81313931 | 2.38497146 |
| 10 | CF807784 | 3.526862338 | 0.75168858 | 5.96195575 |
| 11 | BE807836 | 11.39265251 | 3.19859278 | 1.743448 |
| 12 | CF807852 | 3.418157687 | 1.80999411 | 2.07365181 |
| 13 | AW507968 | 3.104335099 | 2.57047147 | 1.06228435 |
| 14 | CF808510 | 11.48486693 | 2.51601932 | 2.12556985 |
| 15 | CF808574 | 6.774193077 | 1.21492591 | 3.76595519 |
| 16 | CD409075 | 2.893022301 | 3.22692788 | 0.98651507 |
| 17 | CD415193 | 2.82518237 | 1.60014503 | 1.40222319 |
| 18 | BE820446 | 2.634118248 | 2.33678338 | 1.42179684 |
| 19 | BE821438 | 2.543318408 | 1.07485769 | 0.92875609 |
| 20 | BI321576 | 2.207357752 | 0.63989821 | 1.21050888 |
| 21 | BE821939 | 2.355222512 | 0.75568942 | 1.01744913 |
| 22 | BE822796 | 2.095832928 | 2.06451848 | 0.57453114 |
| 23 | BF324082 | 3.416959863 | 2.93603195 | 0.11280892 |
| 24 | BF325482 | 5.267479195 | 2.84297419 | 1.26288389 |
| 25 | BF425742 | 2.068872398 | 0.22402707 | 5.84737453 |
| 26 | BI427426 | 4.769527624 | 0.82651543 | 0.63576272 |
| 27 | BQ628686 | 4.497761581 | 2.56211932 | 0.99246743 |
| 28 | BM731850 | 2.044991104 | 7.95105702 | 0 |
| 29 | BQ741562 | 10.24611681 | 15.9935984 | 1.69791001 |
| 30 | BU544037 | 3.939302141 | 1.60124419 | 2.81553158 |
| 31 | BU545050 | 2.494897545 | 1.32904873 | 2.10737637 |
| 32 | BI945178 | 2.772128801 | 0.92235029 | 11.833886 |
| 33 | BU545579 | 3.055064447 | 0.62824172 | 1.59091674 |
| 34 | BE346777 | 2.151895139 | 5.74552211 | 0.9252839 |
| 35 | BU547499 | 5.270995487 | 0.18070183 | 2.2429669 |
| 36 | BU549025 | 5.875864511 | 4.88986172 | 0.64500951 |
| 37 | AW349551 | 2.153270217 | 0.70421783 | 2.97328413 |
| 38 | BU550139 | 3.139509682 | 0.70494926 | 0.85223744 |
| 39 | AW351262 | 17.11708494 | 7.26594779 | 0.80510266 |
| 40 | BG653183 | 2.017838456 | 1.04722758 | 1.21660345 |
| 41 | AW458014 | 2.091595353 | 3.60212605 | 0.96501459 |
| 42 | BE658881 | 3.954686528 | 0.27741121 | 1.88936137 |
| 43 | AW459852 | 2.172823071 | 0.12099984 | 2.09419822 |
| 44 | BU761457 | 3.897946544 | 18.4130026 | 1.27165266 |
| 45 | BU761764 | 5.880074724 | 1.1706269 | 1.6027114 |
| 46 | CB063558 | 2.30019111 | 5.6008094 | 2.04036275 |
| 47 | BI967585 | 2.27451735 | 1.70729339 | 0.50600516 |
| 48 | BF070218 | 3.582174165 | 2.61411208 | 1.5118947 |
| 49 | BI970890 | 2.476691576 | 1.20762874 | 1.38105521 |
| 50 | BI972938 | 3.803601179 | 1.62313275 | 1.35083956 |
| 51 | BQ473657 | 3.265947707 | 2.62538985 | 2.16894329 |
| 52 | CA783329 | 3.61154719 | 7.7510692 | 0.78218675 |
| 53 | BI784829 | 2.917788554 | 5.49343803 | 0.74028789 |
| 54 | BI786091 | 4.256920675 | 0.55810224 | 14.0406907 |
| 55 | BQ786702 | 6.11243033 | 8.00622041 | 1.8724372 |
| 56 | BM188078 | 5.347282485 | 1.471782 | 0.6766539 |
| 57 | BG790575 | 2.130840142 | 16.3768237 | 0.59244221 |
| 58 | BM891713 | 2.627768053 | 0 | 2.0252528 |
| 59 | CD391920 | 5.01907607 | 9.76984495 | 1.69402246 |
| 60 | BI893143 | 2.349057984 | 0 | 0 |
| 61 | BM094926 | 2.10562882 | 0.37615956 | 0.9078373 |
| 62 | BM094932 | 2.04661982 | 1.66278157 | 1.52008079 |
| 63 | D26092 | Endo control | 1 | 1 |
| 64 | J01298 | Endo control | 1.29685184 | 0.49968529 |

Table 7 lists additional soybean root related, drought related transcription factors that are up- or down-regulated in response to drought condition.

TABLE 7
List of the root related, drought related transcription factors and control
transcripts with the well information

| | | | | |
|---|---|---|---|---|
| | | | Fold | Root |
| Well # | TF name | gene function | Change | Drought |

Preferentially expressed in roots under drought stress

| | | | | |
|---|---|---|---|---|
| 1 | TC205125 | homeodomain transcription factor | 11206.16 | Increase |
| 6 | S15940089 | Zinc finger protein | 4.838342 | Increase |
| 10 | S4864621 | other transcription factor families | 64633.02 | Increase |
| 11 | TC206208 | YABBY2-like transcription factor | 16.8259 | Increase |
| 15 | TC206511 | other transcription factor families | 2.094395 | Increase |
| 16 | S4981395 | other transcription factor families | 287.0654 | Increase |
| 25 | S4914293 | Zinc finger protein | 3.250378 | Increase |
| 32 | S21537971 | other transcription factor families | 6.666005 | Increase |
| 41 | S5142323 | other transcription factor families | 8.709554 | Increase |
| 54 | S21539162 | other transcription factor families | 4.26547 | Increase |
| 55 | TC208789 | MADS box transcription factor | 5.405061 | Increase |
| 62 | S4911726 | putative transcription factor | 1.780905 | Increase |
| 65 | TC209970 | bZIP transcription factor | 4.86728 | Increase |
| 80 | S4898613 | Zinc finger protein | −45.2693 | Decrease |
| 81 | S4875857 | zinc finger protein | 8.182562 | Increase |
| 85 | S4932151 | DNA-binding protein | 15.54086 | Increase |
| 93 | S5146255 | putative transcription factor | 10.16303 | Increase |
| 94 | S4932942 | CHP-rich | 4.51783 | Increase |
| 99 | TC211088 | putative transcription factor | 4.930426 | Increase |
| 103 | TC211951 | MYB domain transcription factor | 8.909314 | Increase |
| 105 | TC211971 | AP2/EREBP, APETALA2/Ethylene-responsive element binding | 25.6248 | Increase |
| | | protein family | | |
| 115 | TC214232 | Cyclic-AMP-dependent transcription factor | 8.449923 | Increase |
| 119 | TC214990 | MYB domain transcription factor | −18.893 | Decrease |
| 126 | S21539727 | homeodomain transcription factor | 6.347033 | Increase |
| 127 | S4885901 | putative transcription factor | 7.898513 | Increase |
| 136 | S21566748 | myb-related protein | −1.74946 | Decrease |
| 140 | S21566080 | Zinc finger protein | 2.456977 | Increase |
| 142 | S21567785 | WRKY domain transcription factor | 5.92074 | Increase |
| 146 | DQ055133 | Glycine max DREB3 | 2.523947 | Increase |
| 147 | TC215663 | other transcription factor families | −2.3001 | Decrease |
| 149 | TC215913 | MYB domain transcription factor | 3.379221 | Increase |
| 151 | TC216048 | other transcription factor families | 7.061372 | Increase |
| 152 | S23070183 | DNA binding protein | 6.046817 | Increase |
| 153 | TC216103 | bZIP transcription factor | −10.9042 | Decrease |
| 162 | S4866988 | other transcription factor families | 73.15146 | Increase |
| 171 | S4925034 | other transcription factor families | 5.185675 | Increase |
| 172 | S21538195 | WRKY domain transcription factor | 44.60338 | Increase |
| 173 | S23070894 | SBP, Squamosa promoter binding protein | −1.52992 | Decrease |
| 175 | S4950242 | DNA-binding protein | 10.8754 | Increase |
| 178 | S21538802 | other transcription factor families | 3.248115 | Increase |
| 179 | S4901375 | EIN3 + EIN3-like(EIL) transcription factor | 17.97298 | Increase |
| 180 | S21540792 | Zinc finger protein | 3.019452 | Increase |
| 190 | S21565790 | putative transcription factor | 5.64075 | Increase |
| 193 | AY974352 | Glycine max NAC4 | −5.82879 | Decrease |
| 200 | S21538617 | MADS box transcription factor | 2.645173 | Increase |
| 201 | TC220047 | putative transcription factor | 4.425233 | Increase |
| 203 | TC220458 | bZIP transcription factor | −2.2654 | Decrease |
| 205 | TC220597 | WRKY domain transcription factor | 5.577539 | Increase |
| 206 | S4912250 | DNA-binding protein | 1.563624 | Increase |
| 209 | TC221650 | bZIP transcription factor | 3.294681 | Increase |
| 222 | S23072065 | MYB domain transcription factor | 10.55804 | Increase |
| 224 | S4896043 | MYB domain transcription factor | 10.08066 | Increase |
| 227 | S4907367 | MADS box transcription factor | 368.2633 | Increase |
| 230 | S23062231 | Zinc finger protein | 1.869604 | Increase |
| 231 | S21539774 | other transcription factor families | −1.78122 | Decrease |
| 238 | S23069233 | putative transcription factor | 4.137847 | Increase |
| 249 | TC225042 | other transcription factor families | 2.196565 | Increase |
| 250 | S4870629 | MYB domain transcription factor | 12.09642 | Increase |
| 251 | TC225047 | other transcription factor families | −4.23604 | Decrease |
| 256 | DQ055134 | Glycine max C2H2 | 8.017523 | Increase |
| 262 | S5129107 | other transcription factor families | 3.352282 | Increase |
| 267 | S15850208 | hunchback protein like | 4.083246 | Increase |
| 272 | S4909265 | putative transcription factor | 15.51433 | Increase |
| 282 | S4911235 | other transcription factor families | 2.575462 | Increase |
| 288 | S22951753 | hunchback protein like | 4.764069 | Increase |
| 292 | S4862202 | other transcription factor families | 2.192659 | Increase |
| 300 | S5146307 | putative transcription factor | 3.136905 | Increase |
| 305 | Z46956 | Glycine max HSTF5 | 2.429612 | Increase |
| 306 | S4904949 | RING zinc finger protein | 4.276327 | Increase |
| 319 | J01298 | Glycine max ACT1 | 3317.992 | Increase |
| 326 | S22952905 | putative transcription factor | 1.838091 | Increase |
| 339 | TC232307 | putative transcription factor | 4.302425 | Increase |
| 341 | TC232363 | MYB domain transcription factor | 10.08527 | Increase |
| 342 | S4877094 | Zinc finger protein | 3.108471 | Increase |
| 343 | TC232817 | putative transcription factor | 1.84859 | Increase |
| 357 | TC235019 | other transcription factor families | −4.2854 | Decrease |
| 359 | | | −4.05153 | Decrease |
| 364 | S21537216 | MYB domain transcription factor | −1.86593 | Decrease |
| 368 | S21540786 | General Transcription | 8.493241 | Increase |
| 374 | S21566054 | G2-like transcription factor, GARP | 3.81518 | Increase |
| 386 | S15849836 | DNA-binding protein | 7.890462 | Increase |
| 387 | S23061430 | LUG | 4.831874 | Increase |
| 388 | S15850391 | other transcription factor families | 5.091384 | Increase |
| 389 | S23061682 | Alfin-like | 3.198659 | Increase |
| 401 | S23063489 | C3H zinc finger | 7.364133 | Increase |
| 407 | S23064915 | CCAAT box binding factor | 4.978799 | Increase |
| 413 | S4877491 | MYB domain transcription factor | 3.24489 | Increase |
| 423 | S4882183 | DNA-binding protein | 3.987868 | Increase |
| 426 | S5002246 | other transcription factor families | 8.419645 | Increase |
| 438 | S18531023 | Zinc finger protein | 3.771058 | Increase |
| 447 | S23067564 | MYB domain transcription factor | 5.655465 | Increase |
| 450 | S21537821 | SET-domain transcriptional regulator family | 3.259263 | Increase |
| 451 | S23068300 | myb-related protein | 9.987982 | Increase |
| 454 | S21538405 | Zinc finger protein | 5.684593 | Increase |
| 456 | S21539619 | other transcription factor families | 7.193817 | Increase |
| 457 | S4884782 | RING zinc finger protein | 2.513477 | Increase |
| 459 | S4884795 | putative transcription factor | 2.273172 | Increase |
| 460 | S5019221 | putative transcription factor | 2.681338 | Increase |
| 461 | S4885448 | other transcription factor families | 4.713803 | Increase |
| 468 | S5026438 | General Transcription | 4.021517 | Increase |
| 471 | S4891443 | bZIP transcription factor | 3.238835 | Increase |
| 486 | S21565183 | bHLH, Basic Helix-Loop-Helix | 2.244631 | Increase |
| 487 | S23070876 | General Transcription | 7.075226 | Increase |
| 489 | S23071068 | TCP transcription factor | 5.322845 | Increase |
| 493 | S23071477 | bHLH, Basic Helix-Loop-Helix | 6.724547 | Increase |
| 504 | S22951976 | Aux/IAA | 5.278411 | Increase |
| 505 | S4895927 | putative DNA-binding protein | 5.299699 | Increase |
| 513 | S4897794 | bHLH, Basic Helix-Loop-Helix | 4.477768 | Increase |
| 518 | S5075763 | HB, Homeobox transcription factor | 17.40339 | Increase |
| 526 | S5076266 | bZIP transcription factor | 14.63446 | Increase |
| 530 | S22952226 | Trihelix, Triple-Helix transcription factor | 3.24605 | Increase |
| 538 | S22953062 | WRKY domain transcription factor | 2.514294 | Increase |
| 540 | S23061205 | Leucine zipper transcription factor | 6.660365 | Increase |
| 541 | S4869132 | TUB transcription factor | 2.039763 | Increase |
| 542 | S23061455 | Aux/IAA | 15.93303 | Increase |
| 546 | S23061550 | bHLH, Basic Helix-Loop-Helix | 4.828178 | Increase |
| 547 | S4875111 | Aux/IAA | 3.263079 | Increase |
| 550 | S23061947 | Trihelix, Triple-Helix transcription factor | 9.147663 | Increase |
| 557 | S4900633 | other transcription factor families | 6.366285 | Increase |
| 558 | S5088770 | other transcription factor families | 3.60347 | Increase |
| 559 | S4901877 | other transcription factor families | 3.414657 | Increase |
| 564 | S5100831 | Zinc finger protein | 1.990323 | Increase |
| 567 | S4904547 | other transcription factor families | 1.98464 | Increase |
| 570 | S5103646 | Agamous like | 4.954743 | Increase |
| 578 | S23062909 | bHLH, Basic Helix-Loop-Helix | 12.34281 | Increase |
| 584 | S23063261 | myb-related protein | 15.35067 | Increase |
| 592 | S23064130 | General Transcription | 4.930358 | Increase |
| 596 | S23064932 | MYB domain transcription factor | 3.246497 | Increase |
| 598 | S23065007 | other transcription factor families | 7.825335 | Increase |
| 599 | S4888307 | ARR | 4.308908 | Increase |
| 603 | S4908810 | C2H2 zinc finger | 3.976952 | Increase |
| 606 | S5130128 | DNA-binding protein | 9.46924 | Increase |
| 607 | S4910460 | MYB domain transcription factor | 3.567659 | Increase |
| 609 | S4910851 | EIN3 + EIN3-like(EIL) transcription factor | 1.553793 | Increase |
| 620 | S5146158 | bZIP transcription factor | 12.02518 | Increase |
| 621 | S4913507 | Zinc finger protein | 3.82379 | Increase |
| 625 | S4891278 | bHLH, Basic Helix-Loop-Helix | 3.25324 | Increase |
| 627 | S4891674 | MADS box transcription factor | 2.409738 | Increase |
| 629 | S4892093 | AP2/EREBP, APETALA2/Ethylene-responsive element binding | −3.3456 | Decrease |
| | | protein family | | |
| 630 | S23066857 | Bromodomain proteins | 8.293166 | Increase |
| 640 | S23070418 | C2H2 zinc finger | 10.62733 | Increase |
| 653 | S4917467 | Zinc finger protein | 24.3013 | Increase |
| 655 | S4917546 | MYB domain transcription factor | 3.082696 | Increase |
| 666 | S6675518 | putative transcription factor | 4.461472 | Increase |
| 674 | S23071935 | other transcription factor families | 3.704373 | Increase |
| 678 | S4861946 | AP2/EREBP, APETALA2/Ethylene-responsive element binding | 2.403874 | Increase |
| | | protein family | | |
| 688 | S4867907 | putative transcription factor | 103.7044 | Increase |
| 698 | S5035170 | EIN3 + EIN3-like(EIL) transcription factor | 3.675418 | Increase |
| 707 | S4948369 | Zinc finger protein | 15.55212 | Increase |
| 711 | S4953170 | other transcription factor families | 5.62144 | Increase |
| 718 | S5126262 | MYB domain transcription factor | 9.556359 | Increase |
| 721 | S4980774 | Chromatin remodeling complex subunit | 11.08125 | Increase |
| 723 | S4981647 | ARF, Auxin Response Factor | 6.775763 | Increase |
| 726 | S4872717 | DNA-binding protein | 3.506245 | Increase |
| 728 | S4872880 | other transcription factor families | 8.086666 | Increase |
| 740 | S4875903 | WRKY domain transcription factor | 7.377872 | Increase |
| 744 | S4876683 | ARF, Auxin Response Factor | 4.451186 | Increase |
| 745 | S4967941 | MADS box transcription factor | 4.636514 | Increase |
| 753 | S4976159 | AT-rich interaction domain containing transcription factor | 8.441762 | Increase |
| 755 | S4980388 | Chromatin remodeling complex subunit | 1.940131 | Increase |
| 764 | S5146871 | Aux/IAA | −4.69505 | Decrease |
| 164 | AY974349 | Glycine max NAC1 | 34.31886 | Increase |
| 199 | DQ028773 | Glycine max NAC5 | 5.514578 | Increase |
| 720 | S5146166 | NAC domain transcription factor | 3.189606 | Increase |
| 177 | AY974351 | Glycine max NAC3 | 1.004904 | Similar |
| 704 | S5050636 | NAC domain transcription factor | 3.678247 | Increase |
| 165 | DQ028770 | Glycine max NAC2 | 2.248117 | Increase |
| 204 | DQ028774 | Glycine max NAC6 | 16.47516 | Increase |
| 384 | S22952239 | NAC domain transcription factor | 12.28312 | Increase |
| 501 | S4863935 | CCAAT box binding factor | 10.82859 | Increase |

Preferentilally expressed in roots

| | | |
|---|---|---|
| 3 | TC205627 | bZIP transcription factor |
| 7 | TC205929 | AP2 transcription factor like |
| 14 | S4930680 | DNA-binding protein |
| 17 | TC206902 | AP2 transcription factor like |
| 18 | S4882983 | MYB domain transcription factor |
| 22 | S4966677 | EIN3 + EIN3-like(EIL) transcription factor |
| 24 | S4904584 | WRKY domain transcription factor |
| 50 | S5011331 | other transcription factor families |
| 83 | S5046001 | MYB domain transcription factor |
| 90 | S4981738 | Zinc finger protein |
| 123 | S4879817 | Zinc finger protein |
| 130 | DQ054363 | Glycine max DREB2 gene |
| 155 | TC216155 | bZIP transcription factor |
| 191 | S23068684 | bZIP transcription factor |
| 215 | TC223128 | WRKY domain transcription factor |
| 244 | S5045942 | Zinc finger protein |
| 259 | TC225723 | WRKY domain transcription factor |

House keeping/controls

Gmub12
UBI
Tub
ELF
Scof

### Example 10

### Sequences of Soybean Transcription Factors Belonging to the Different Families

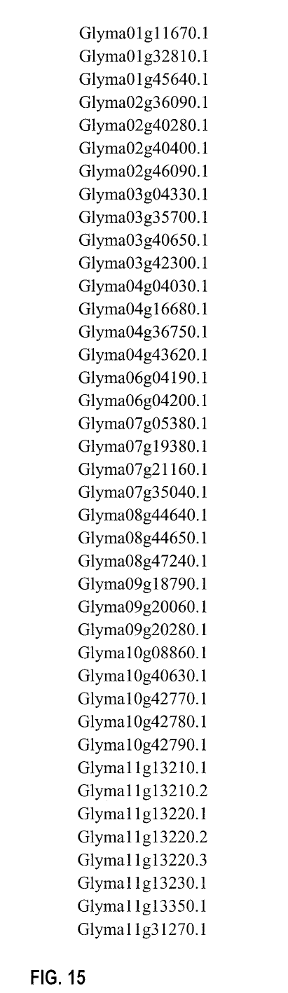

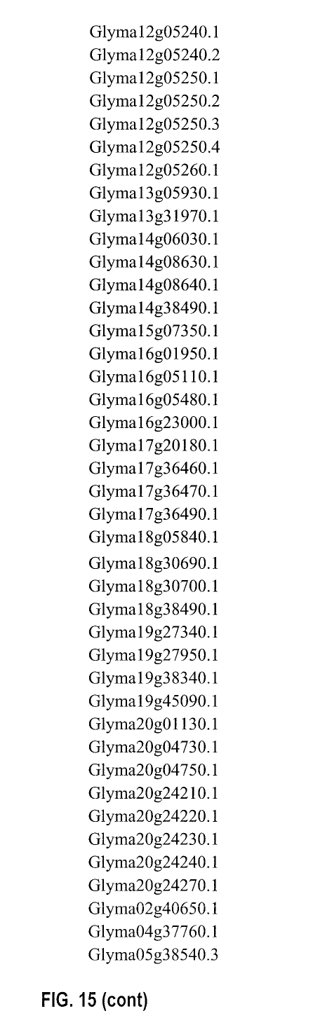

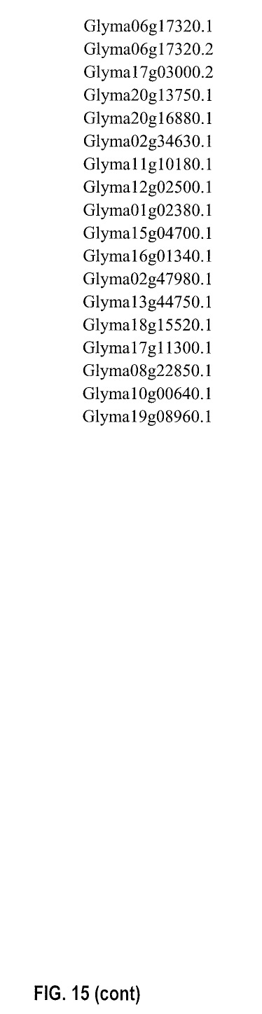

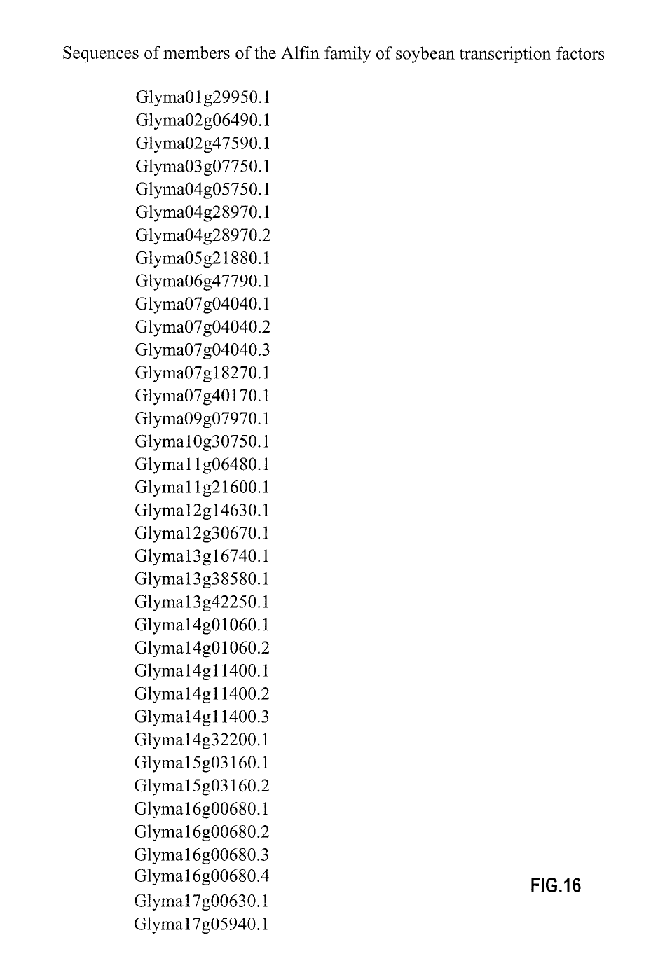

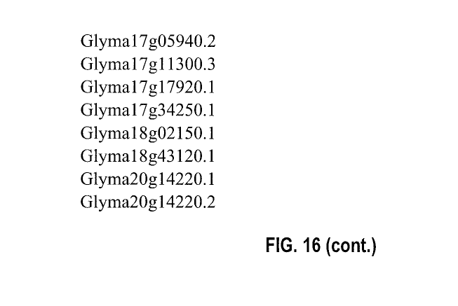

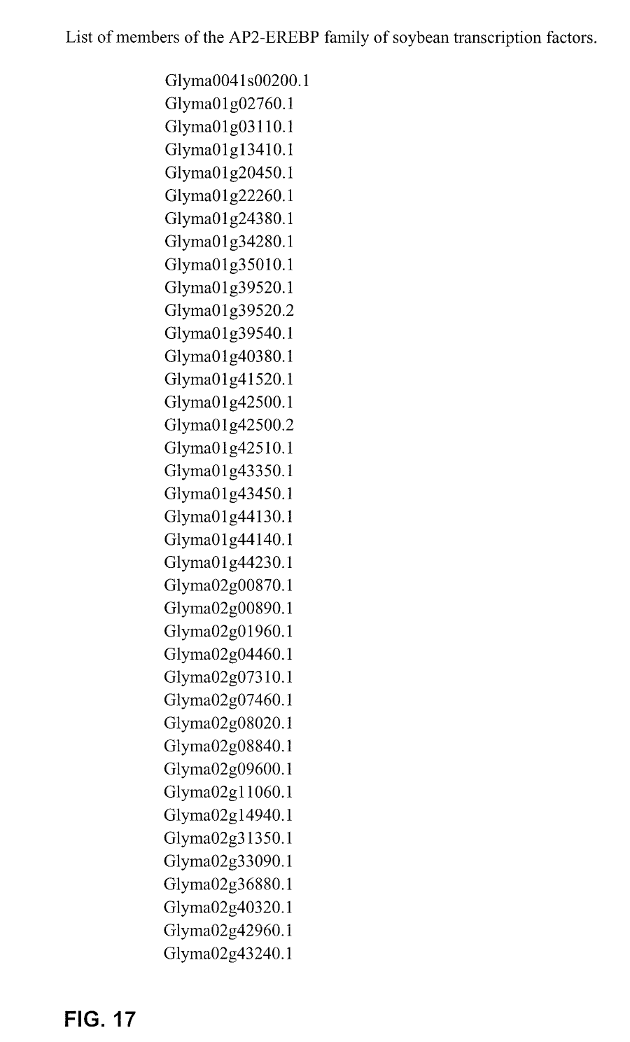

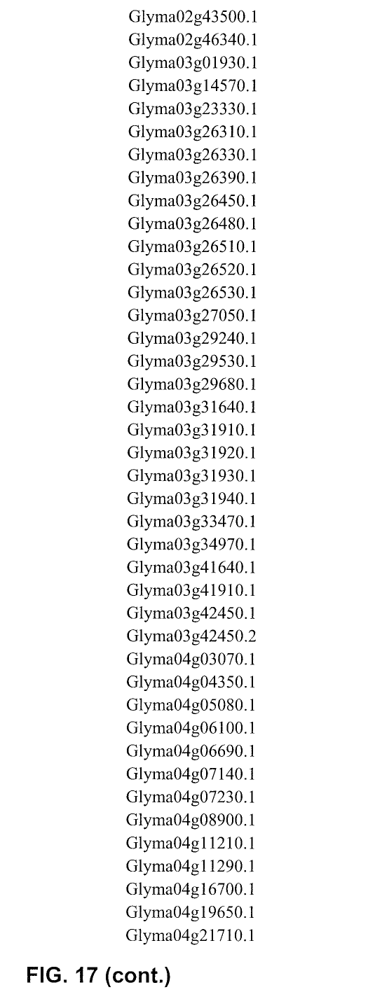

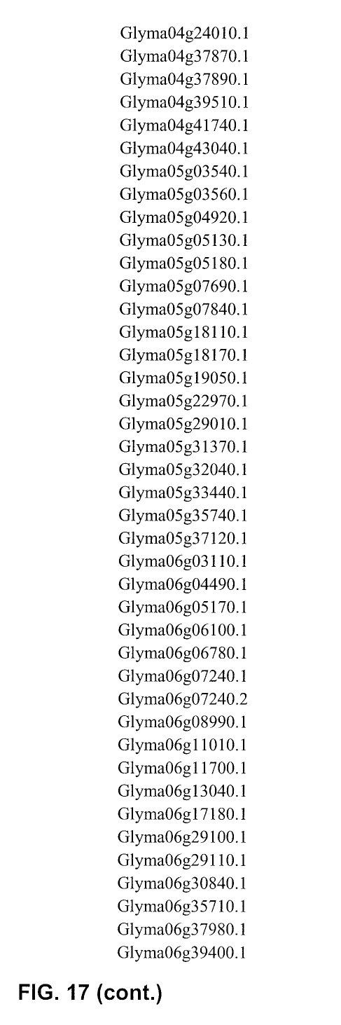

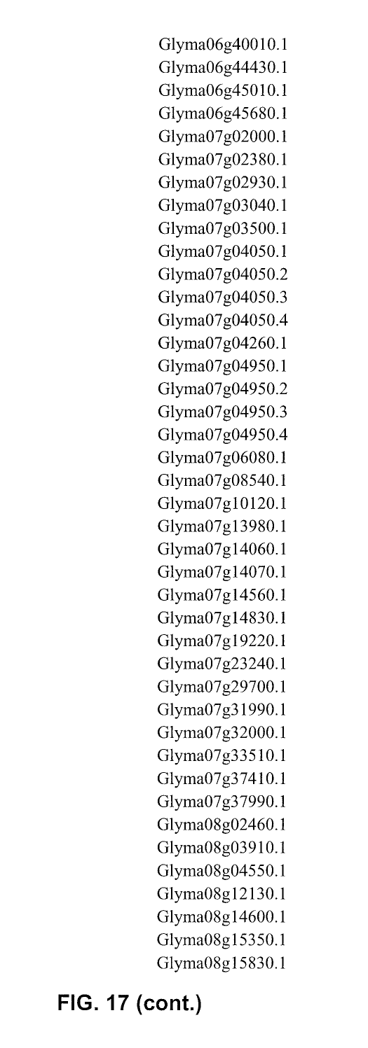

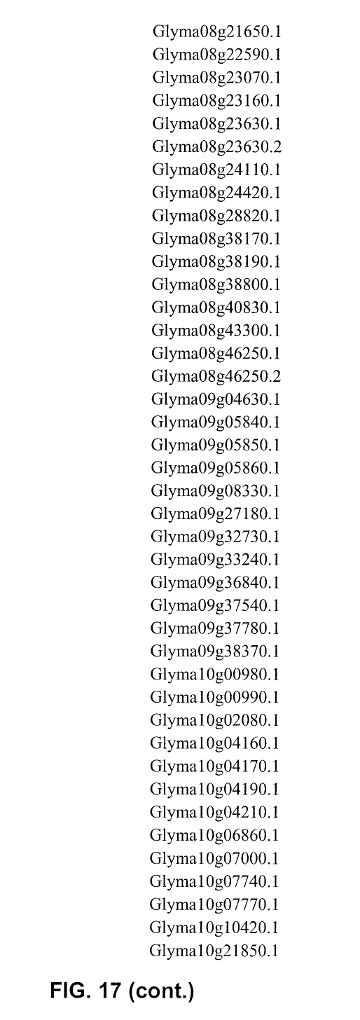

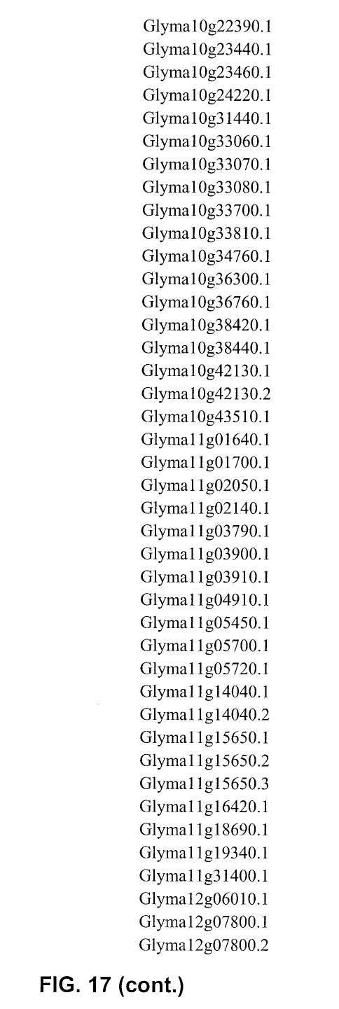

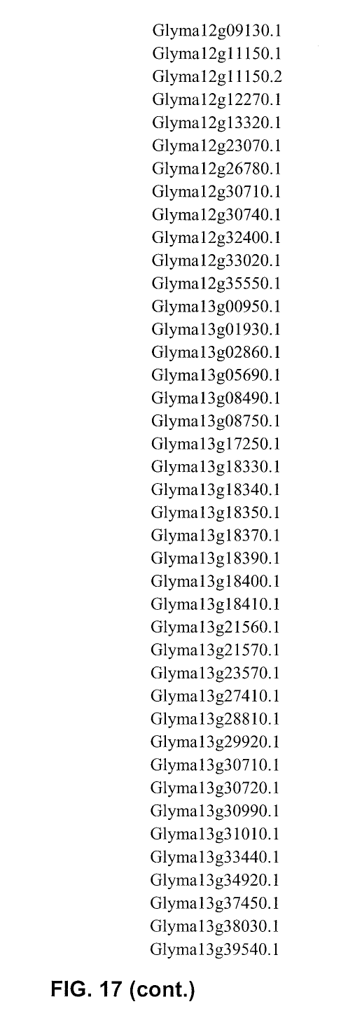

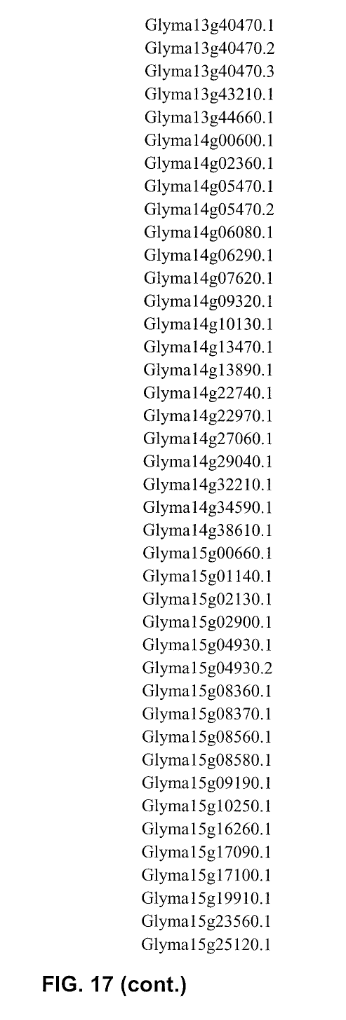

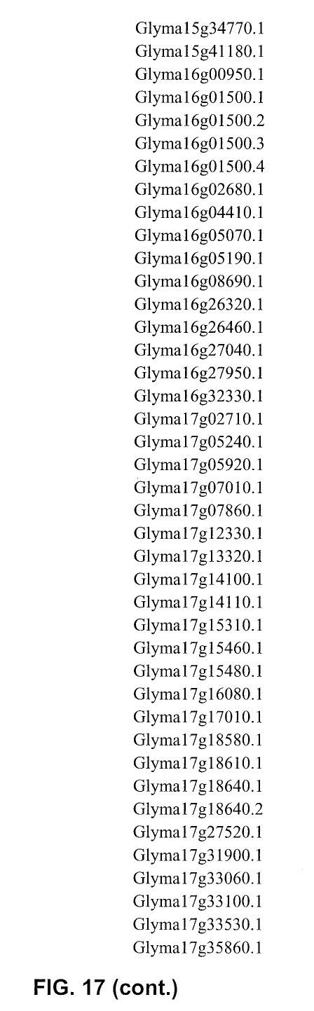

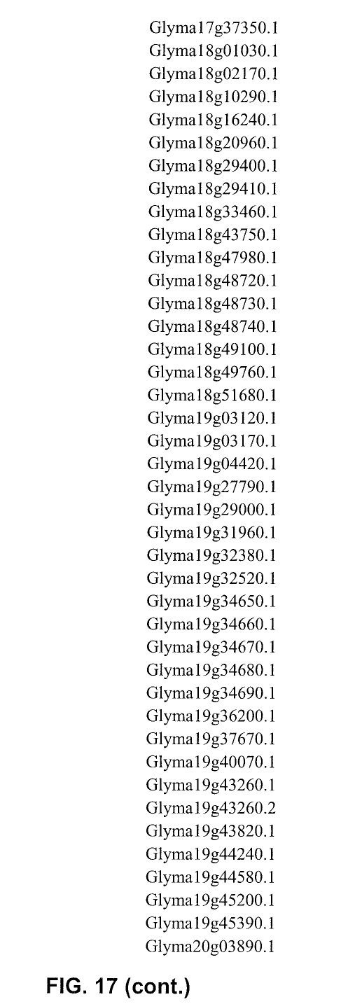

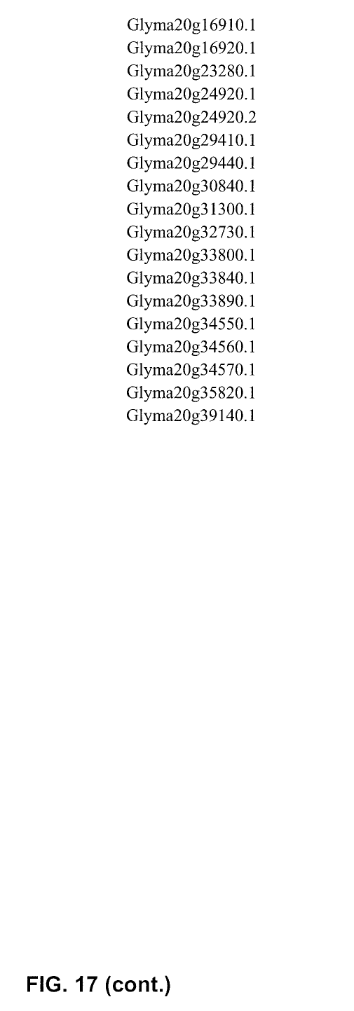

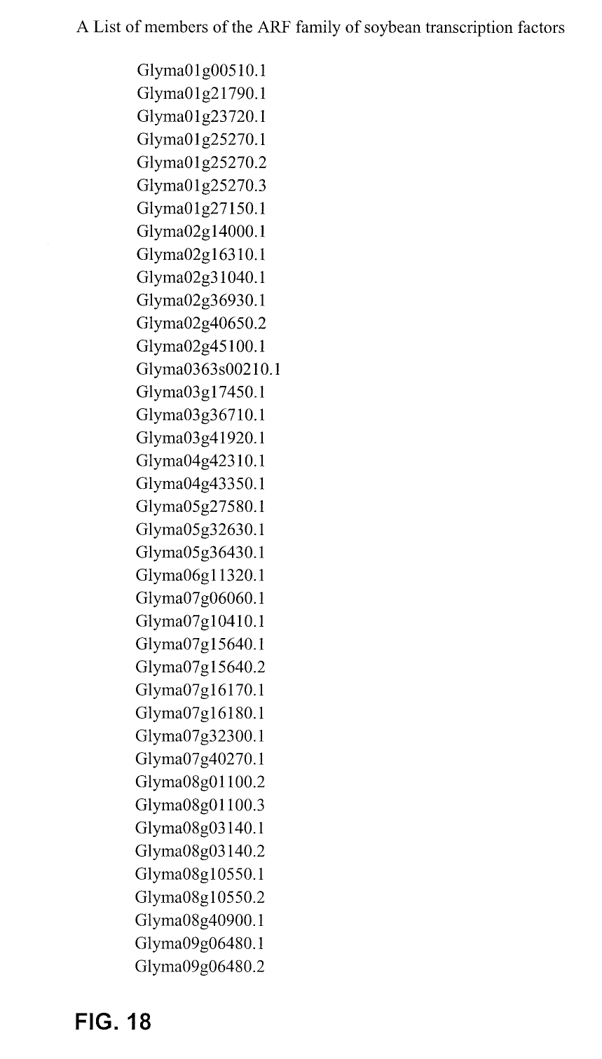

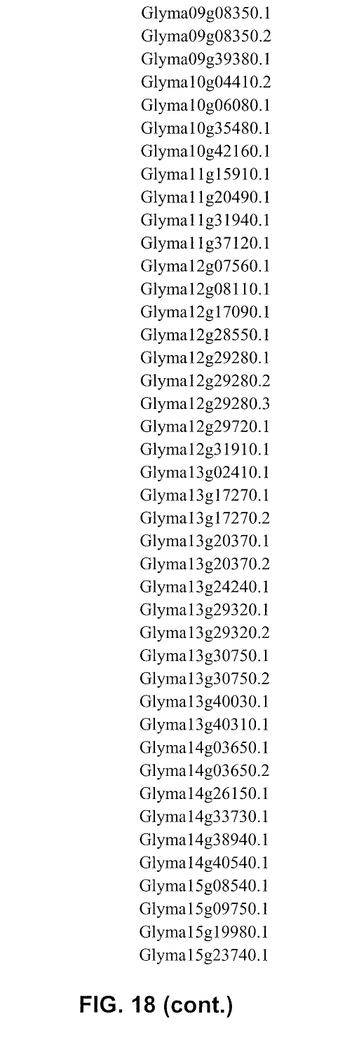

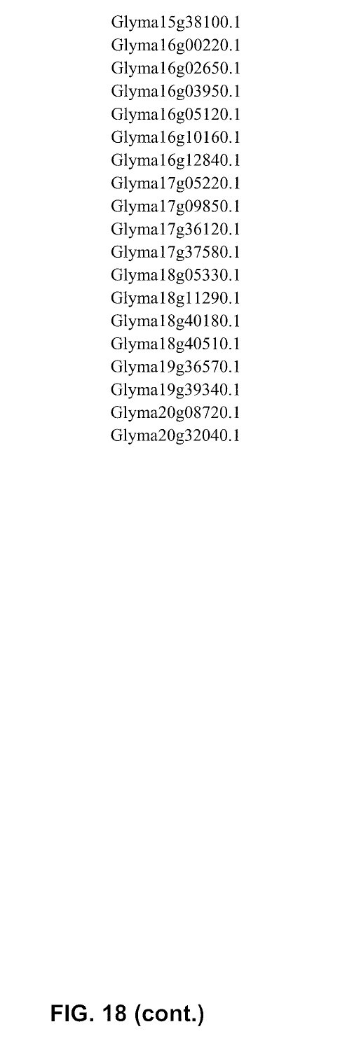

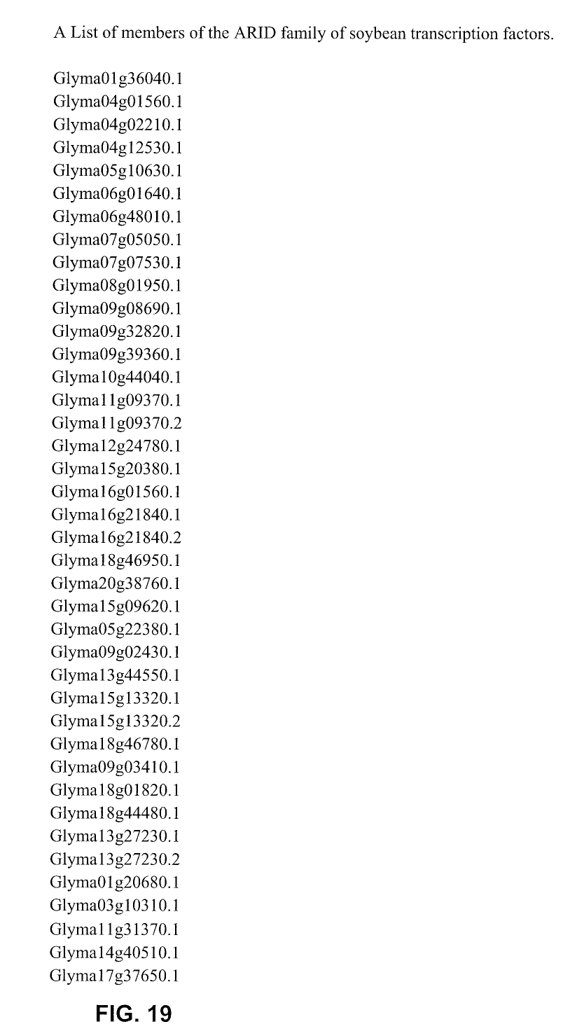

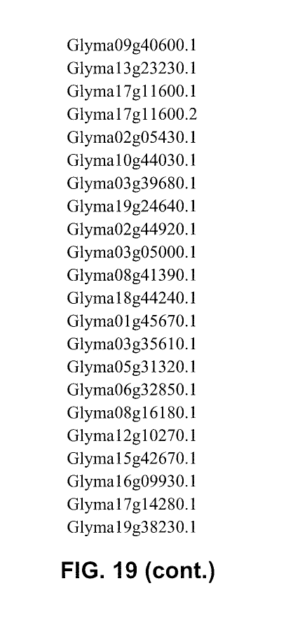

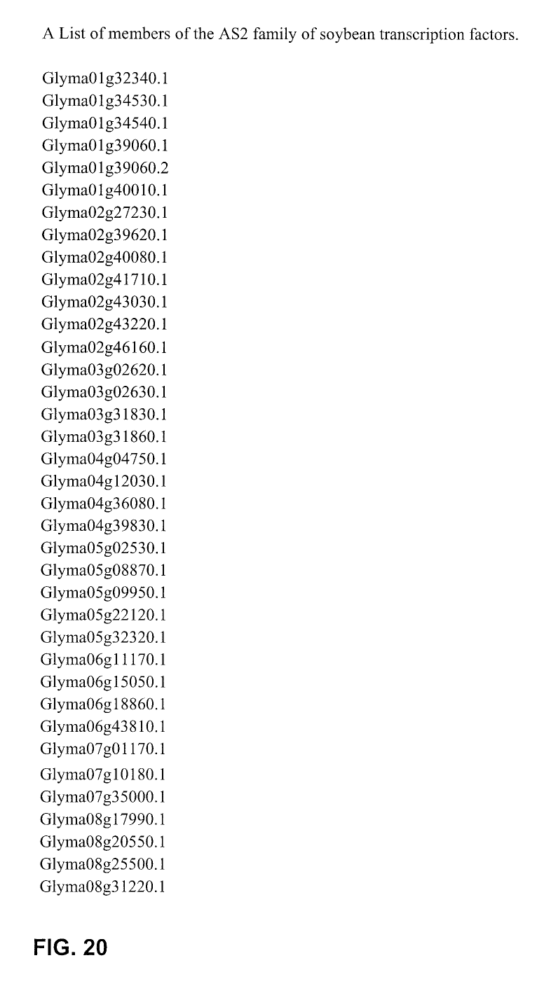

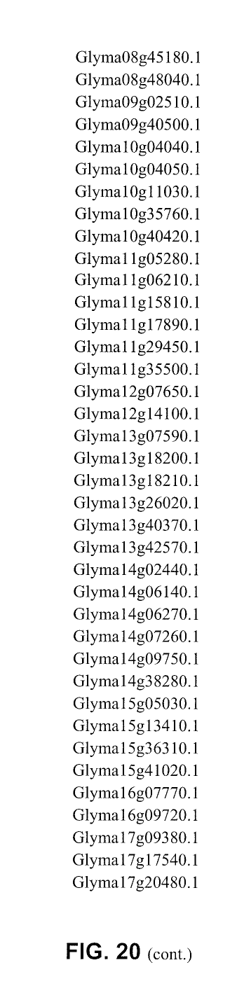

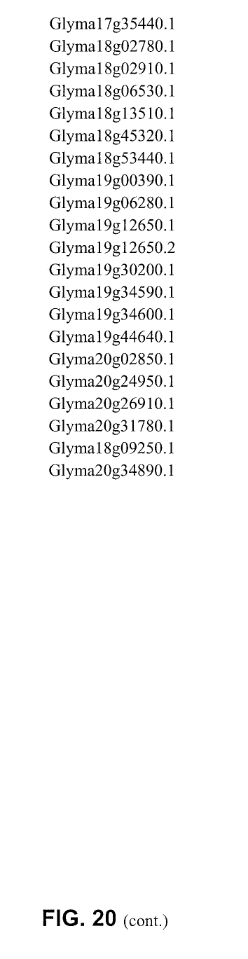

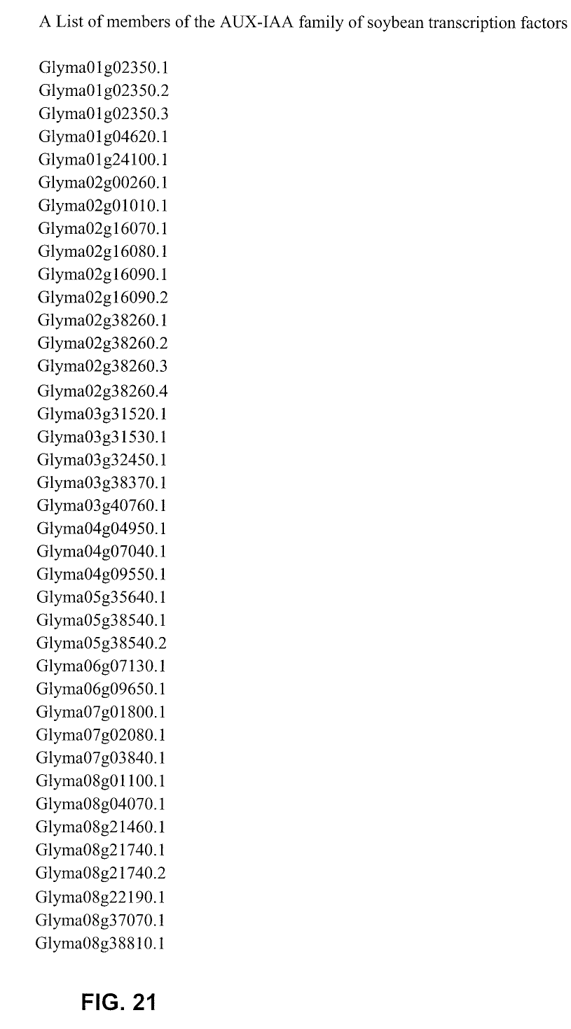

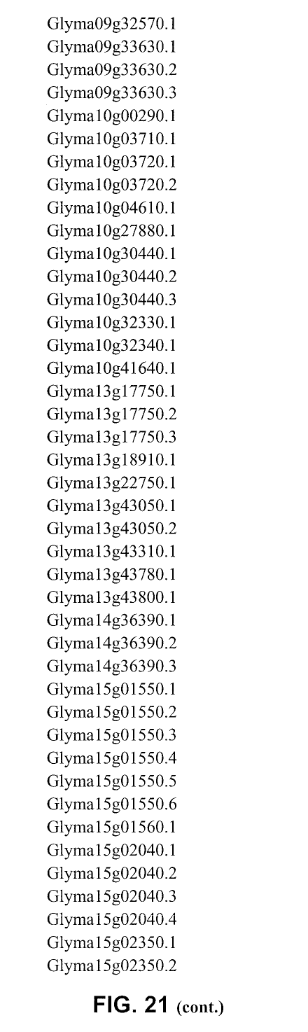

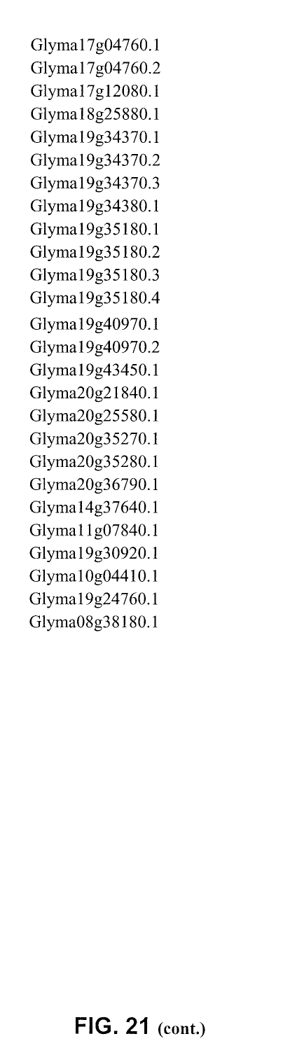

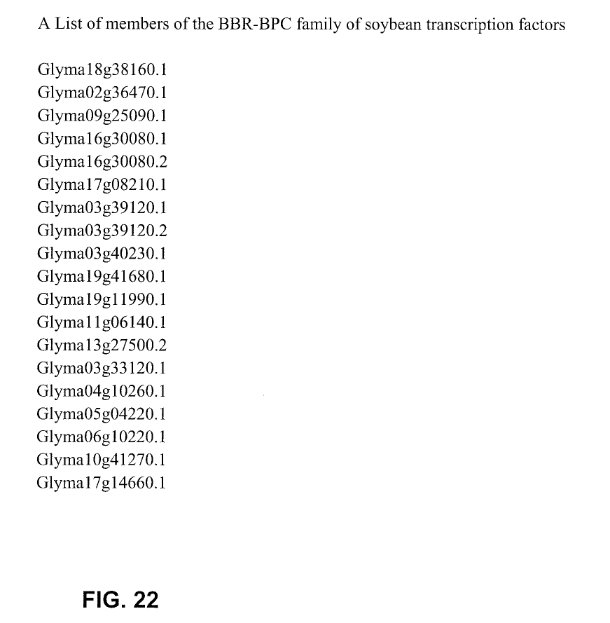

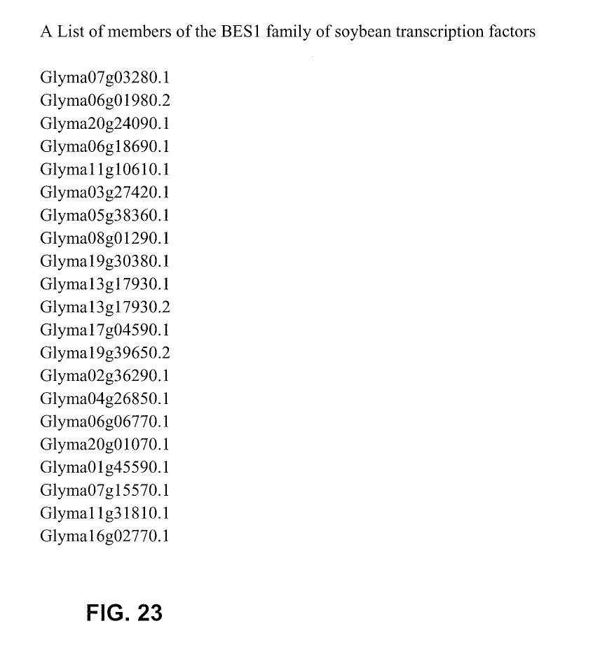

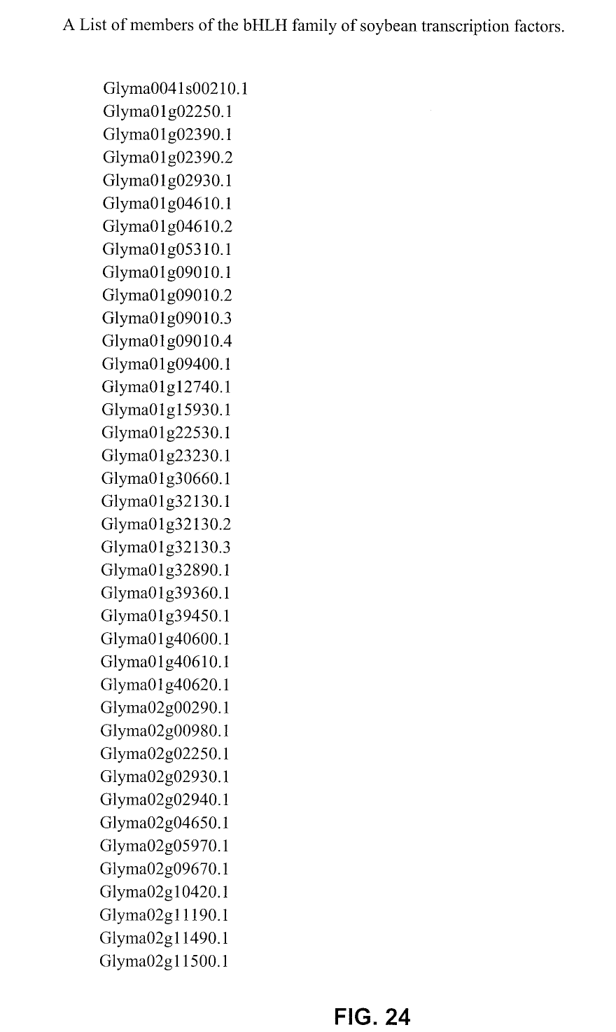

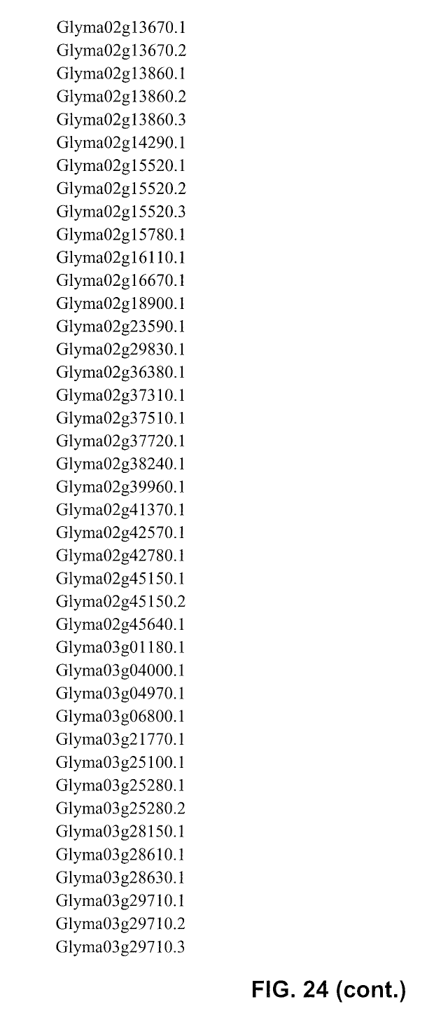

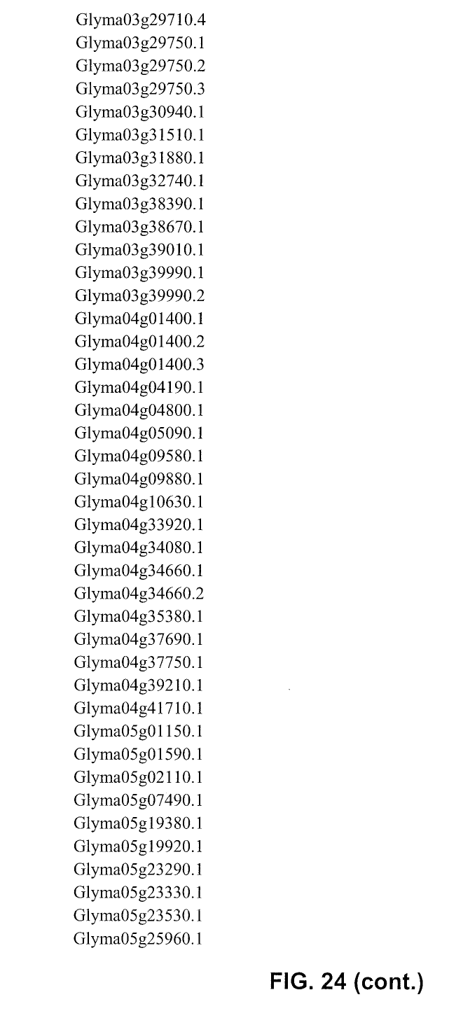

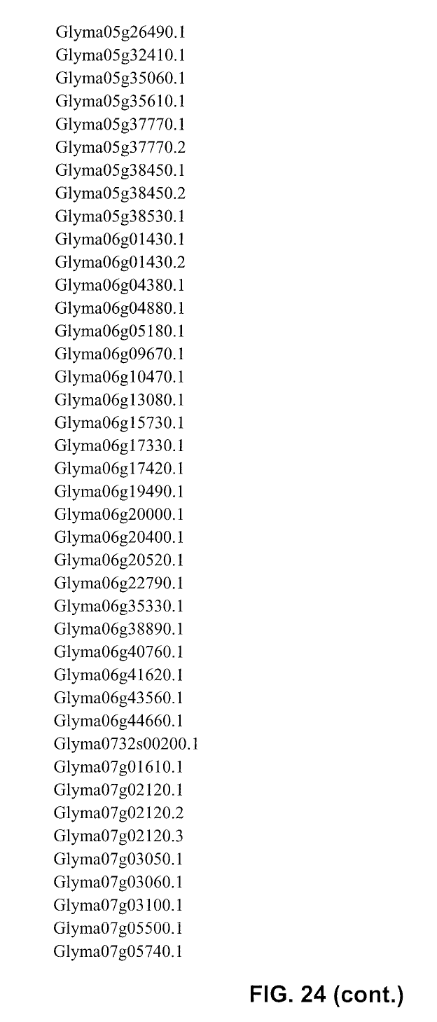

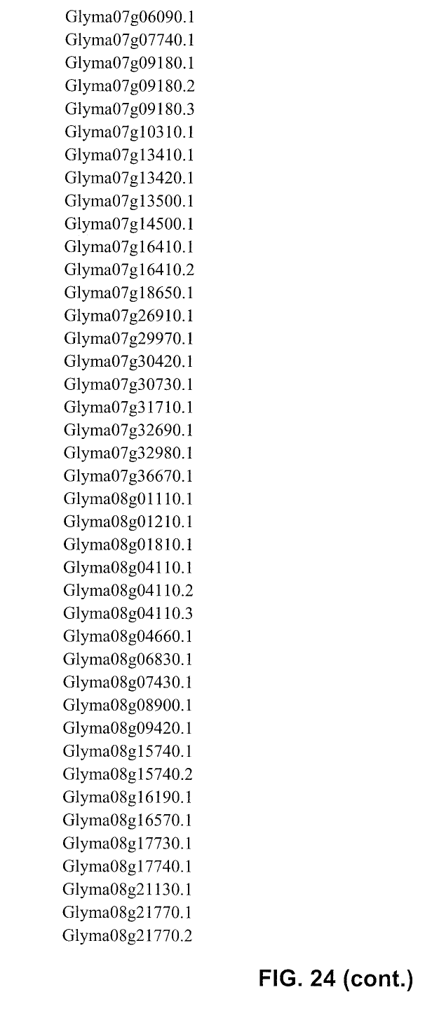

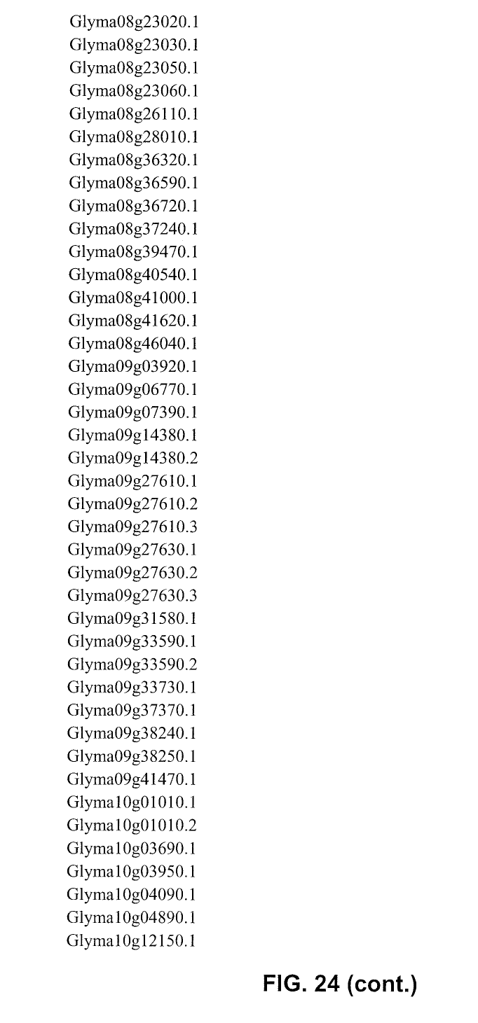

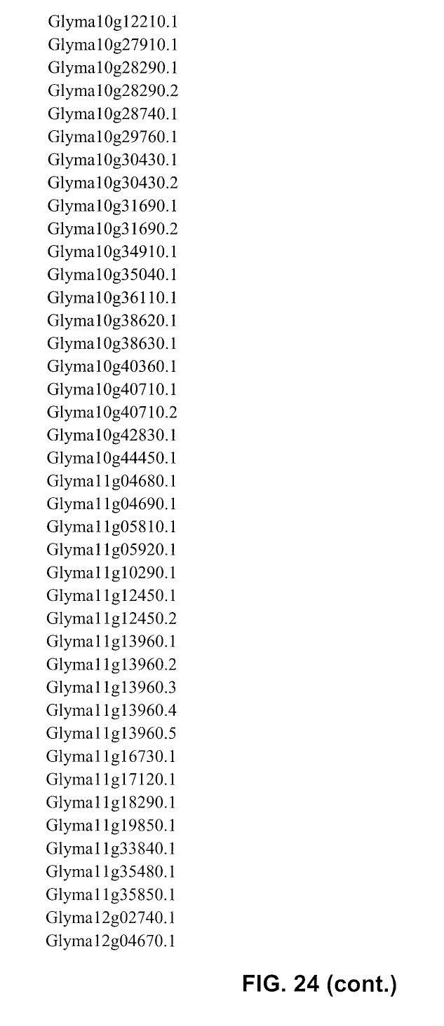

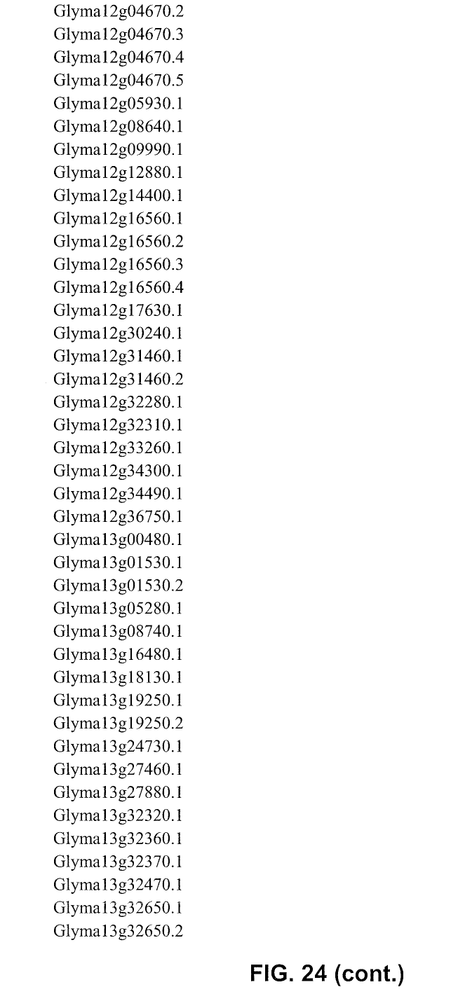

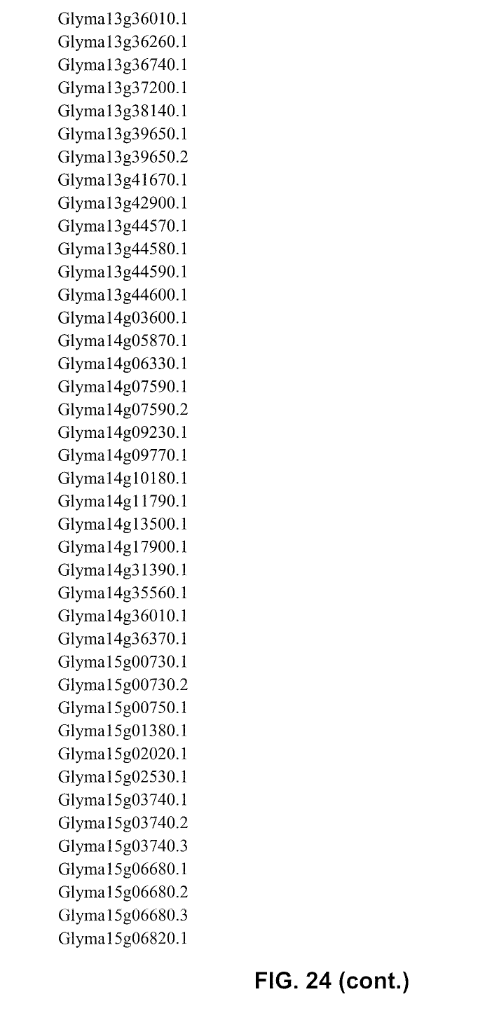

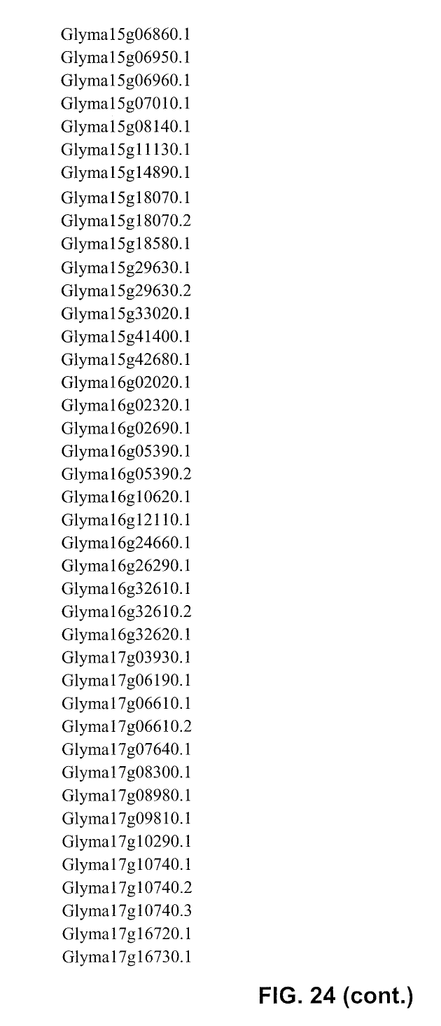

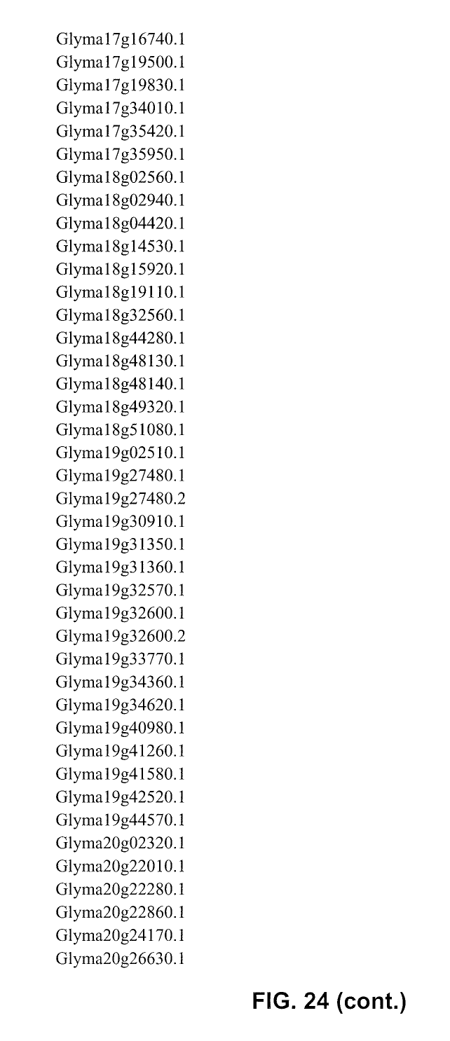

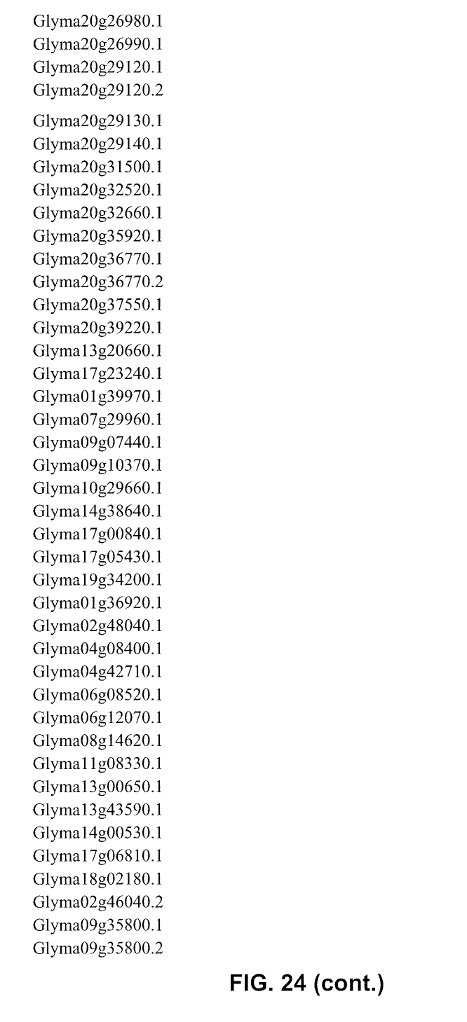

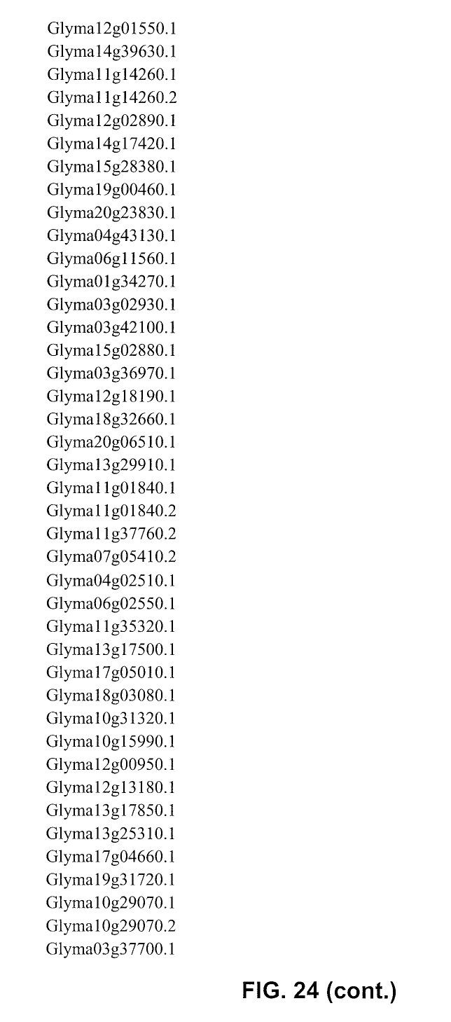

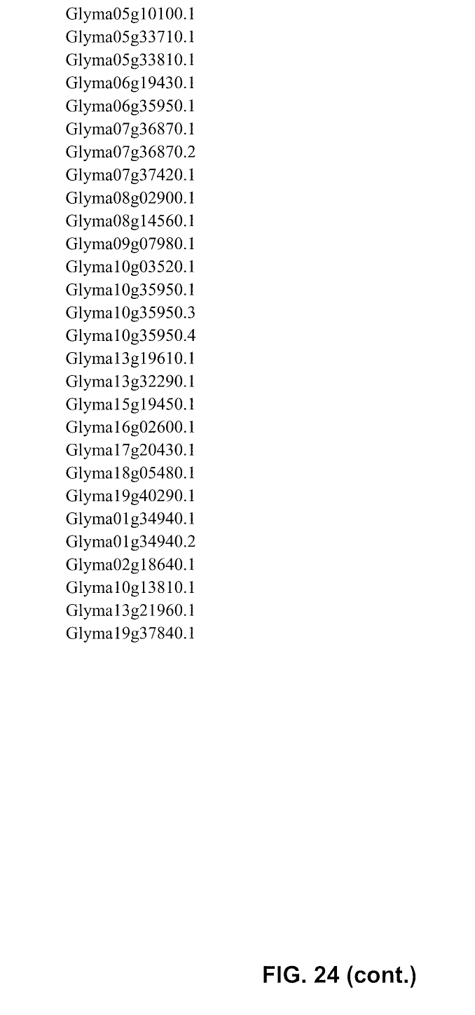

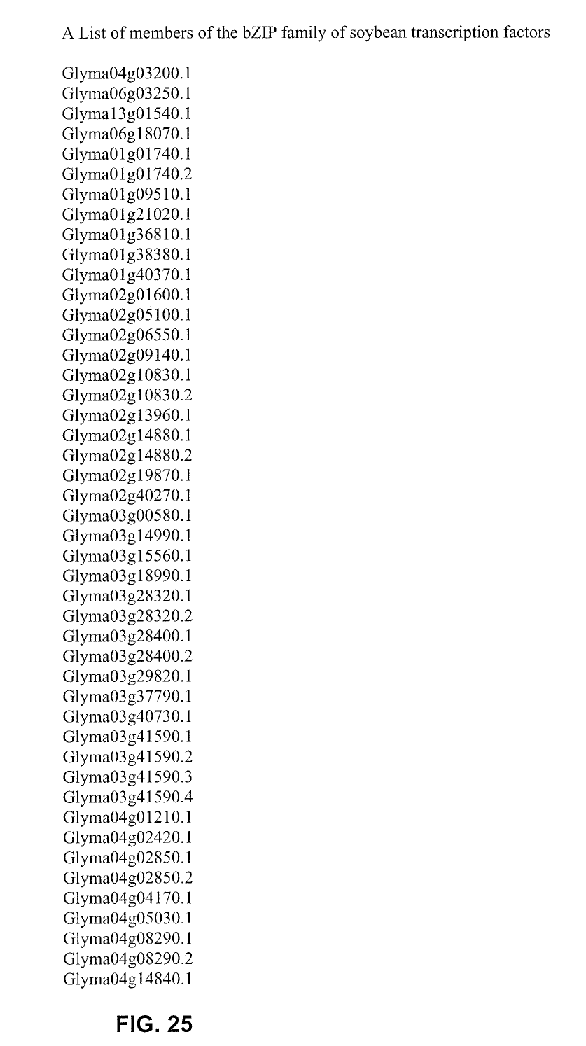

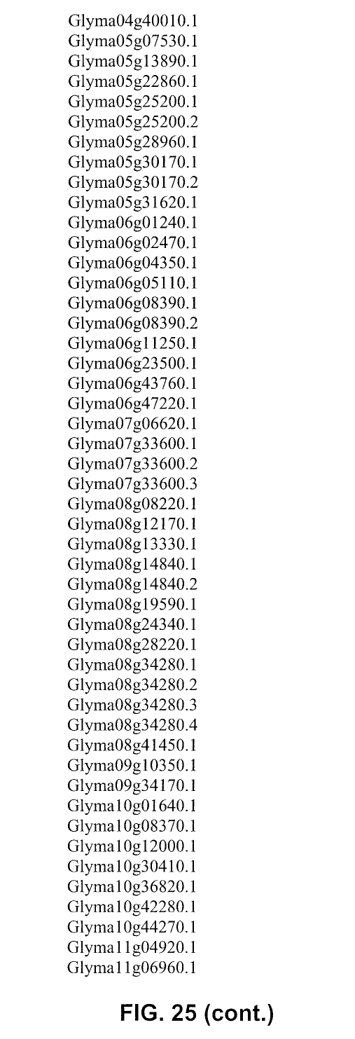

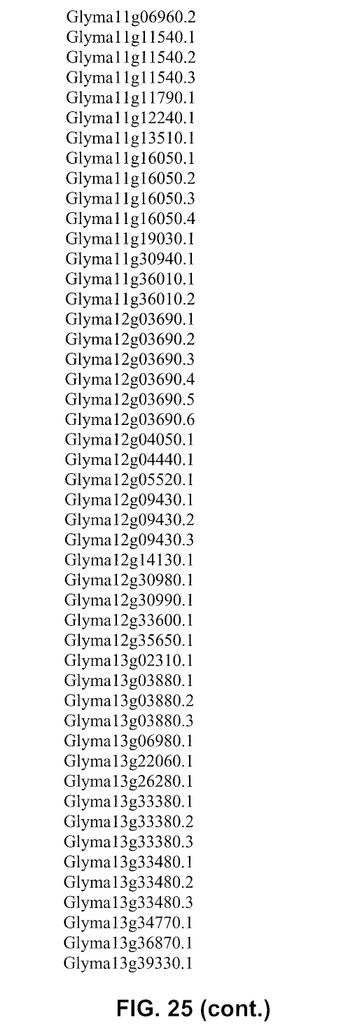

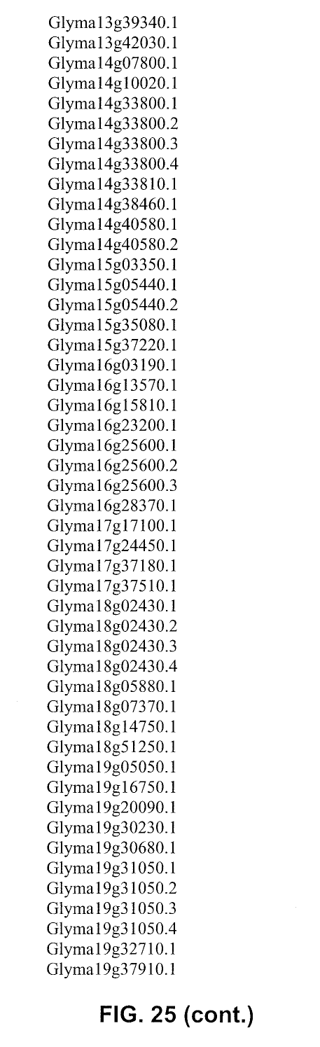

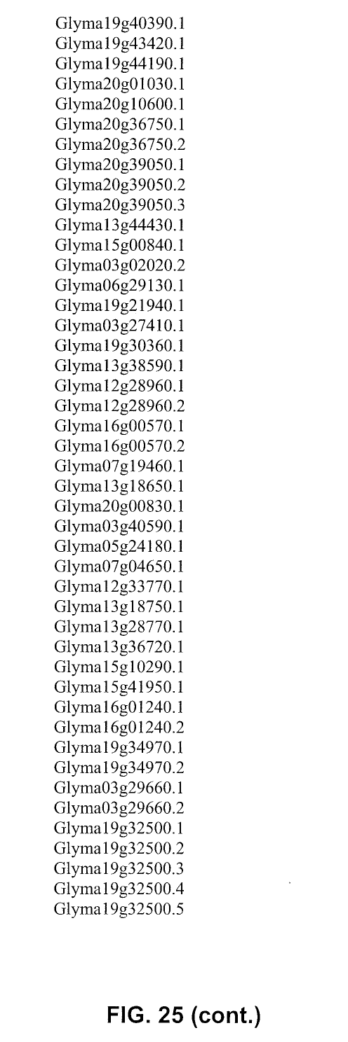

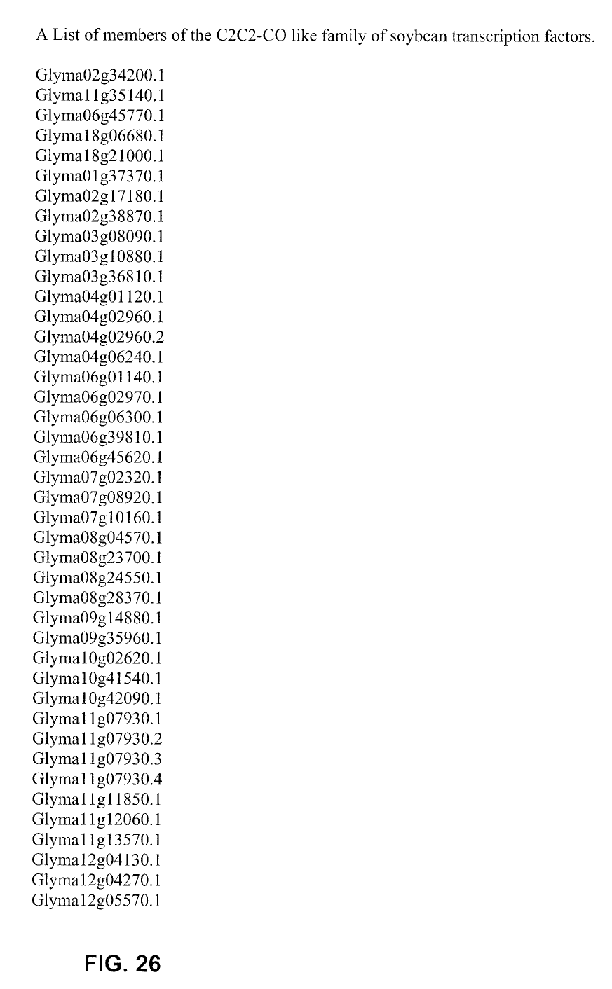

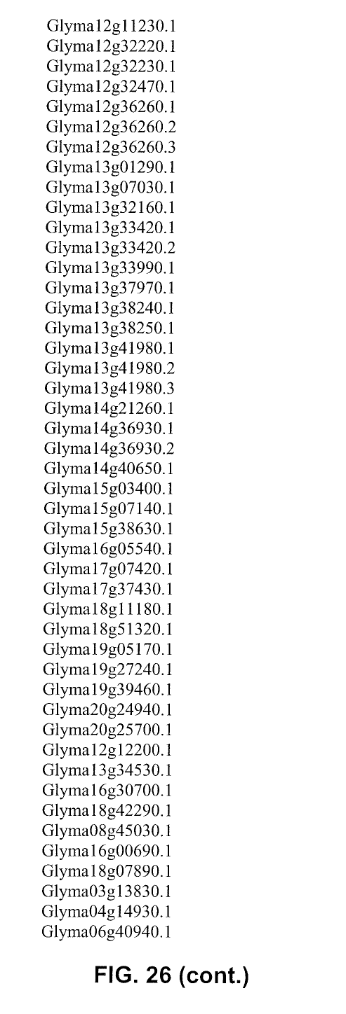

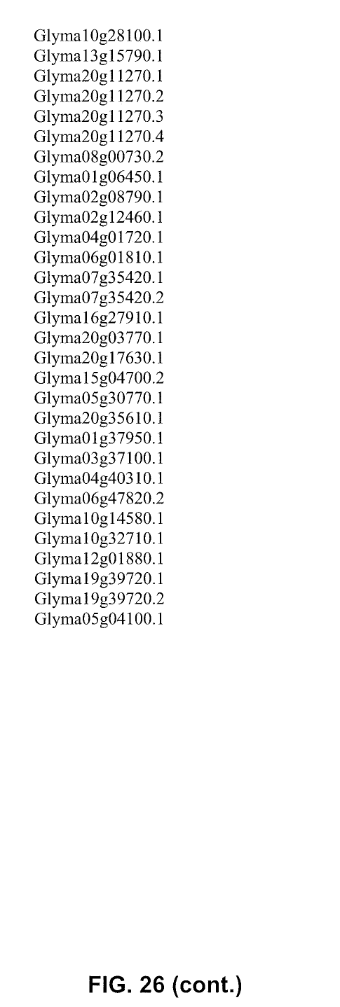

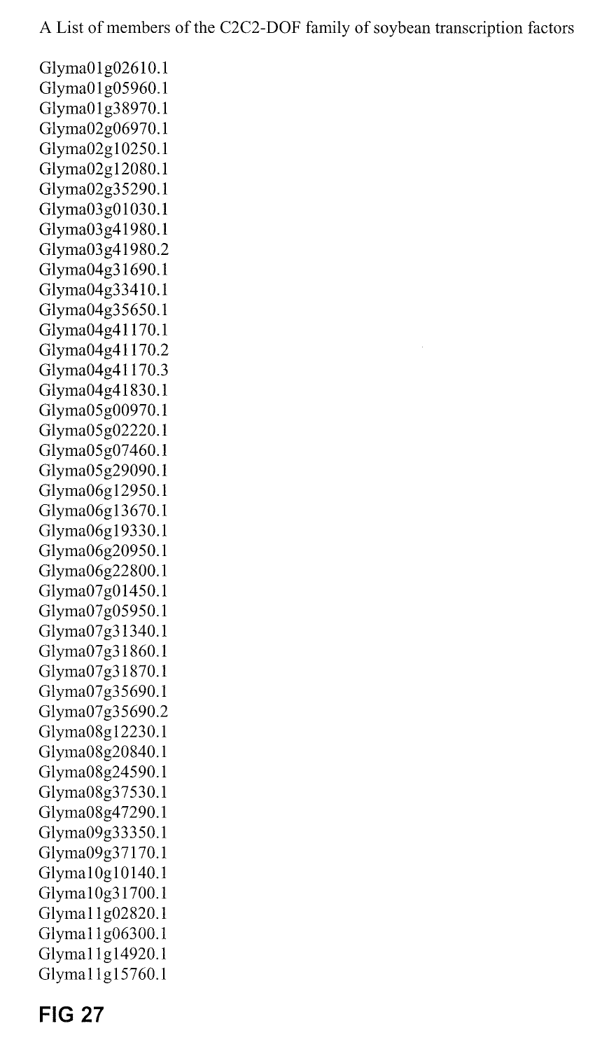

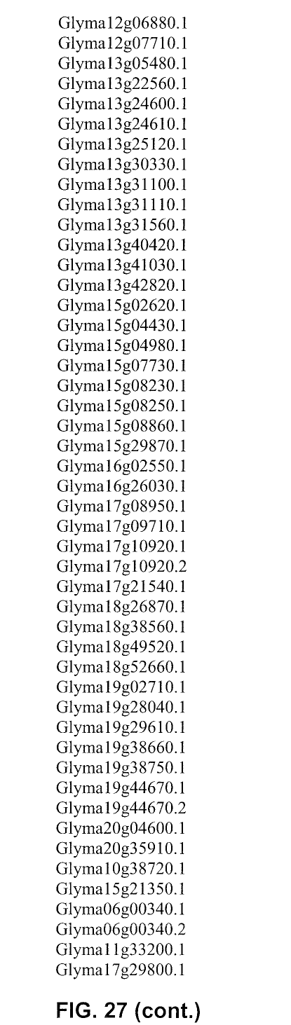

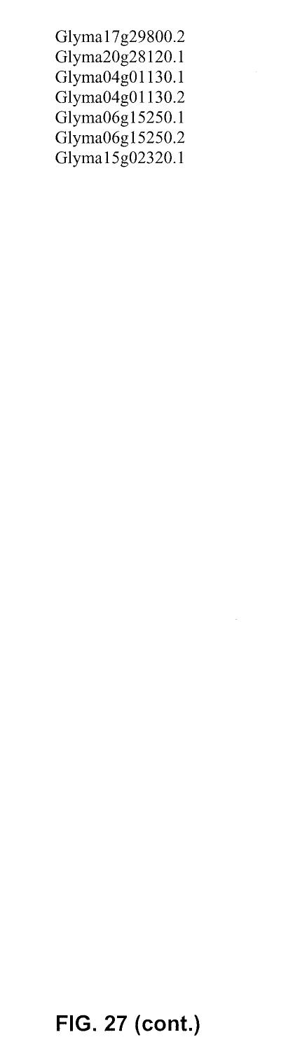

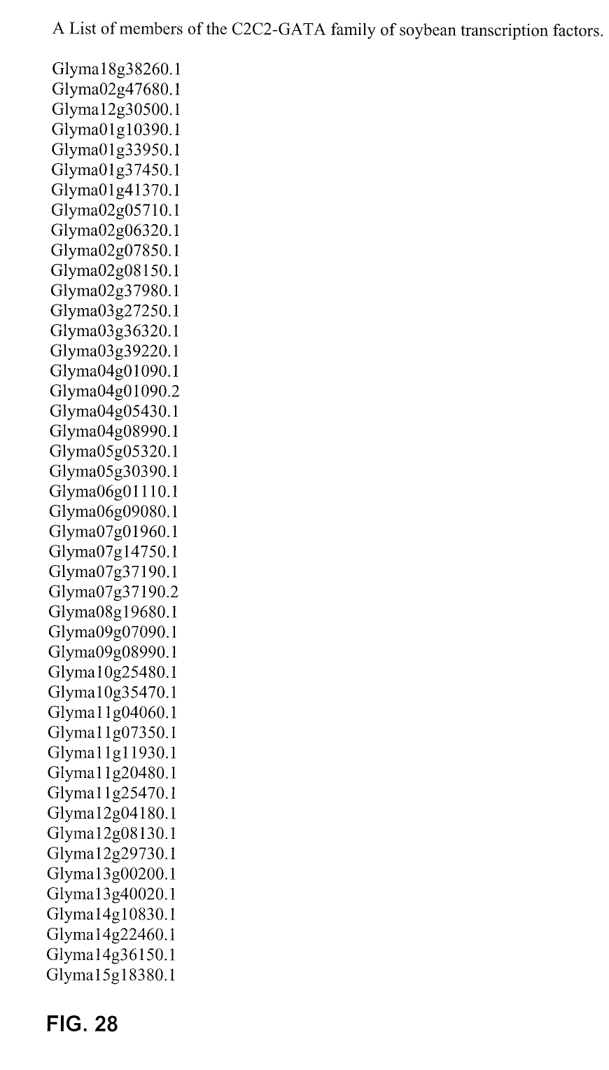

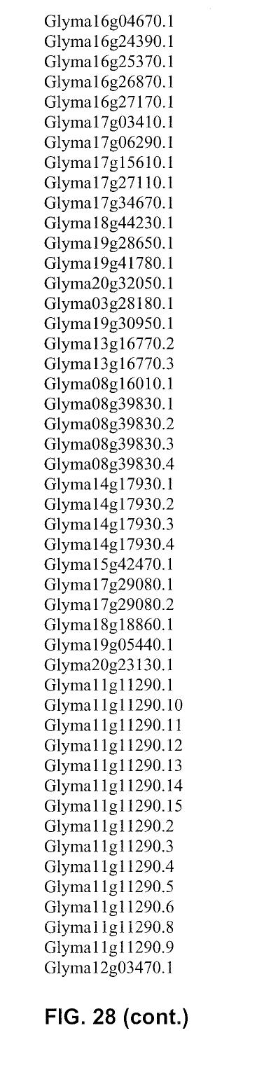

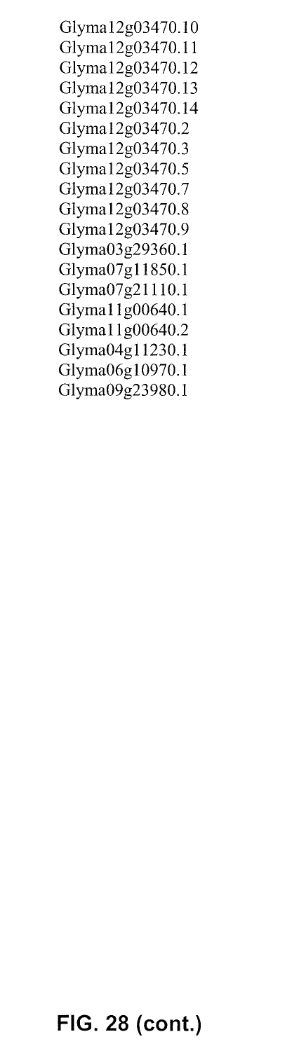

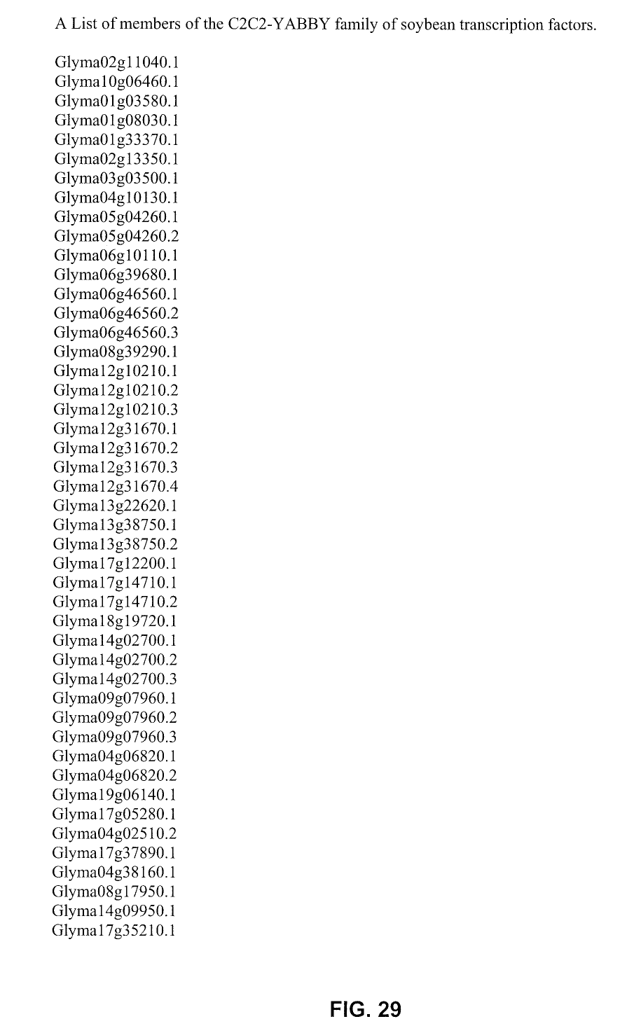

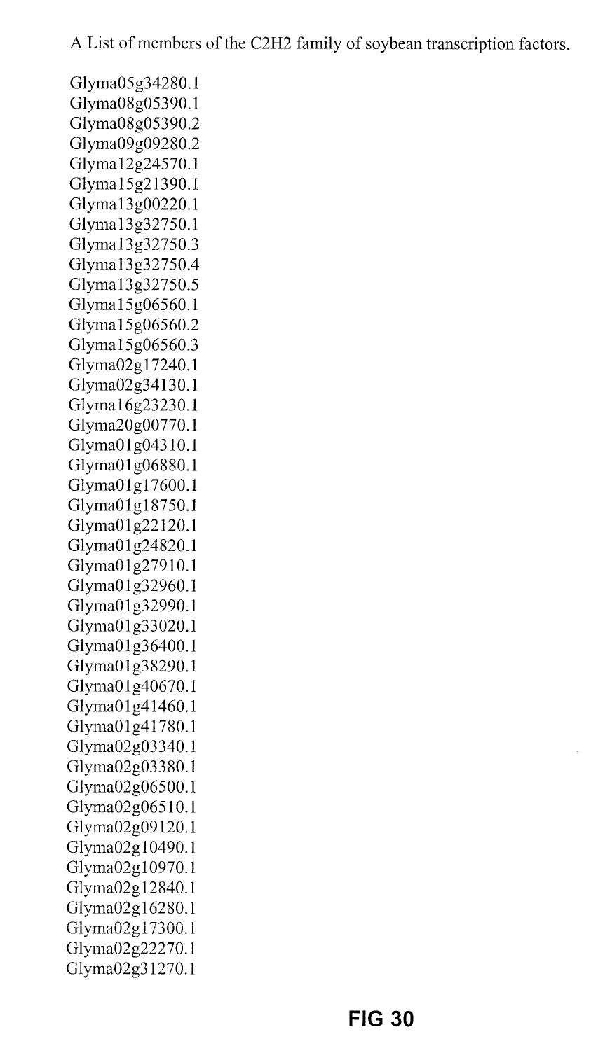

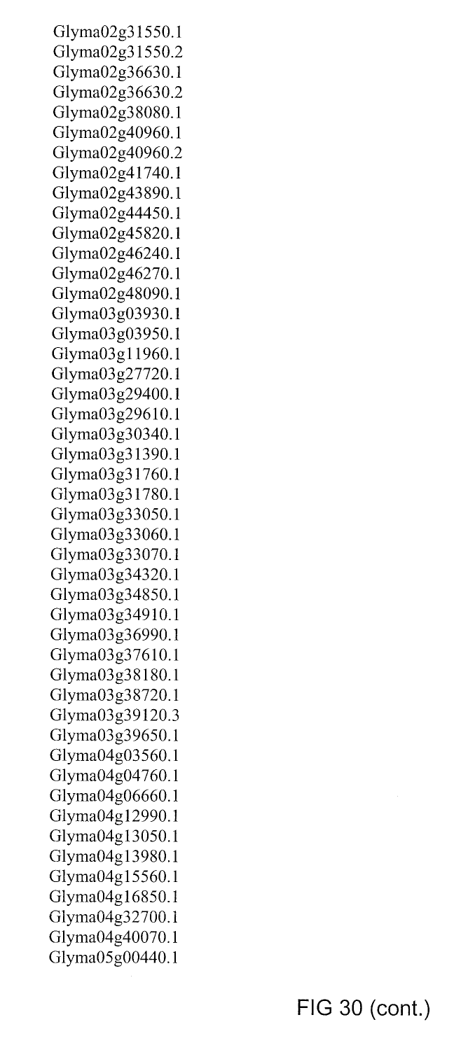

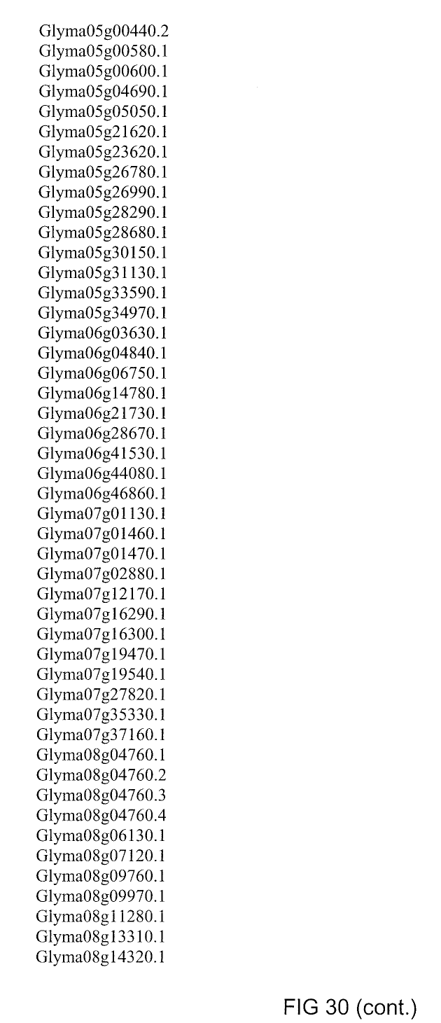

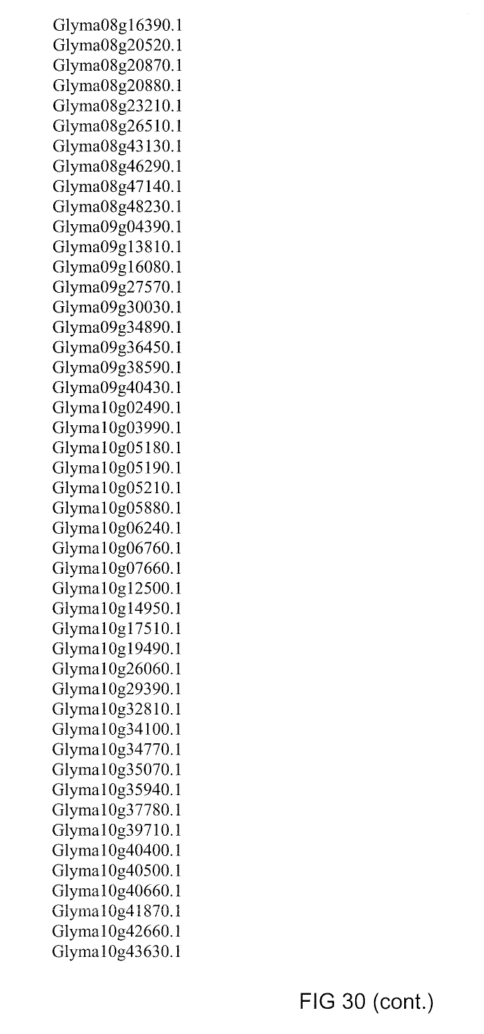

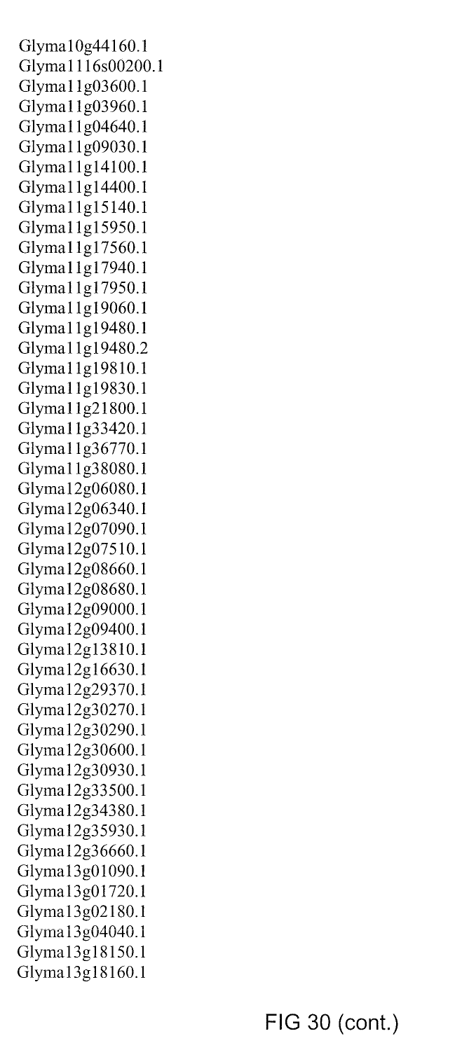

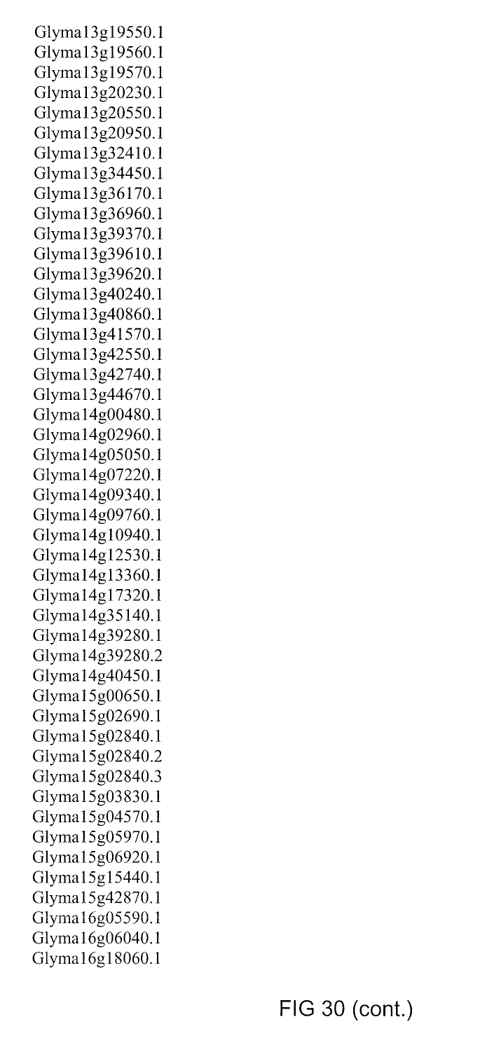

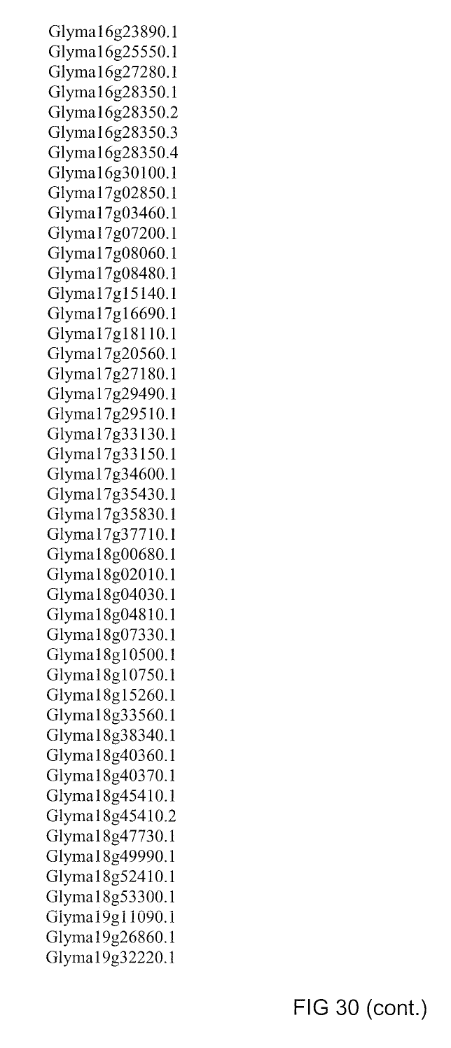

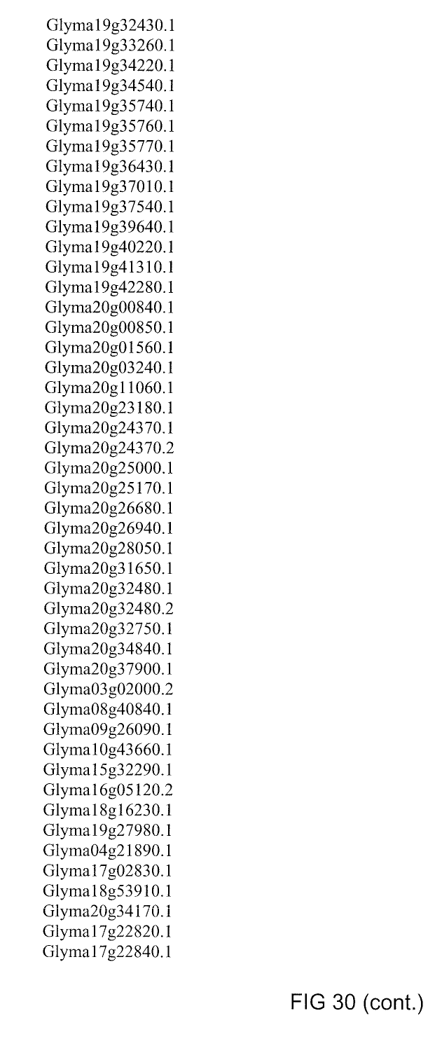

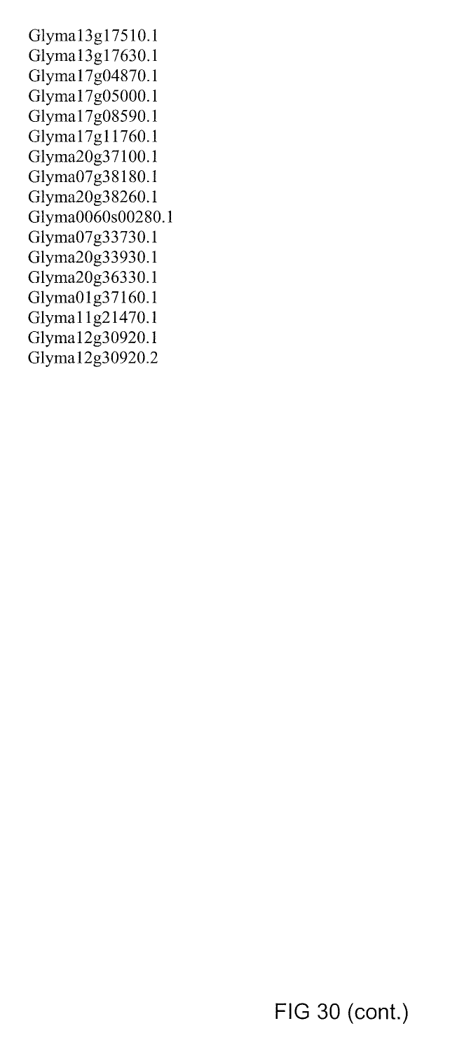

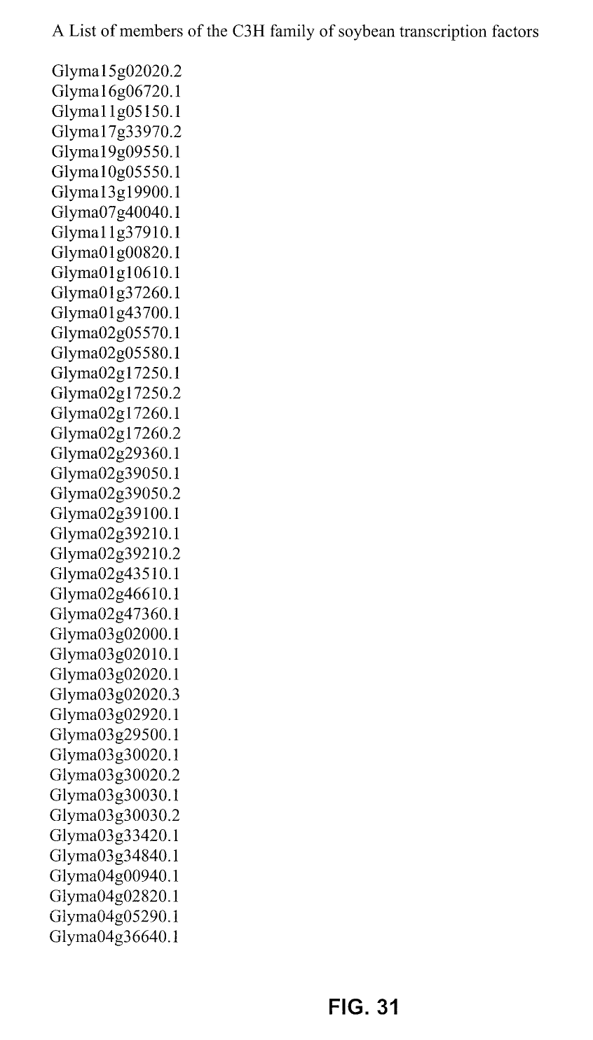

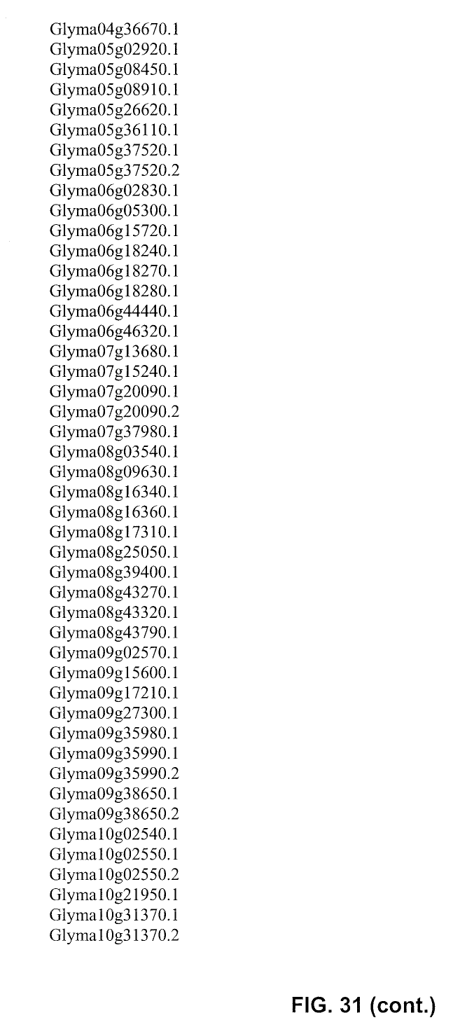

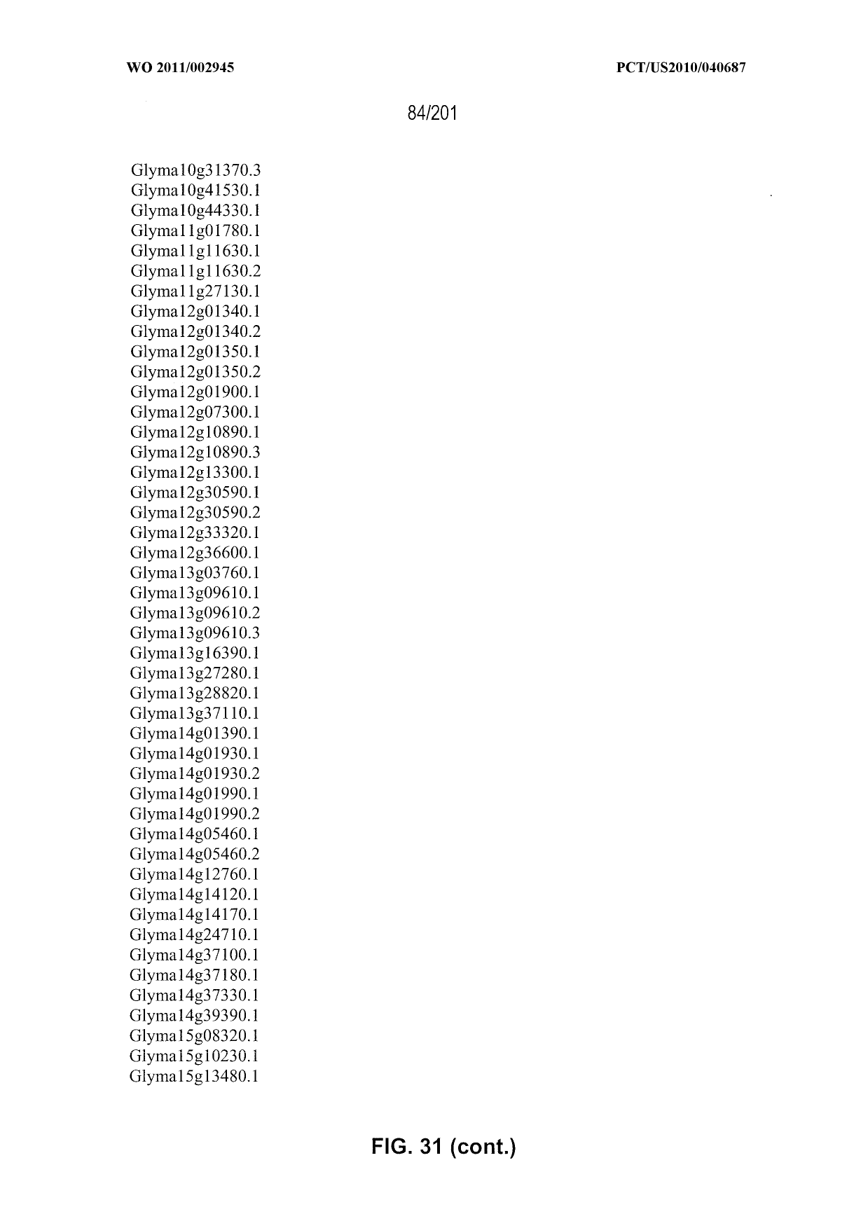

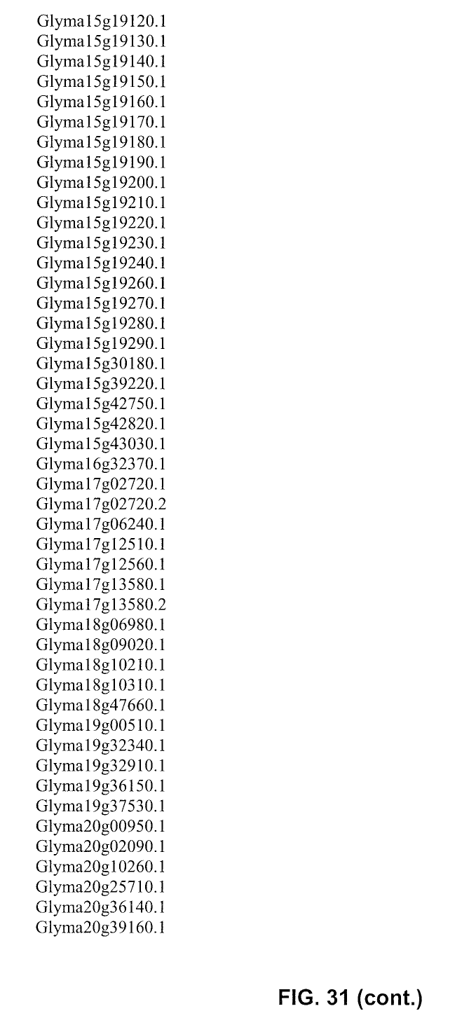

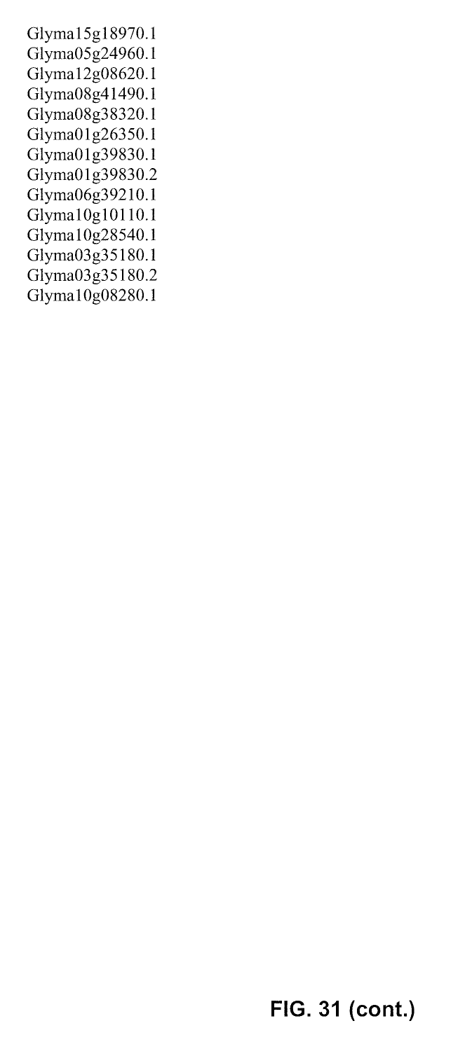

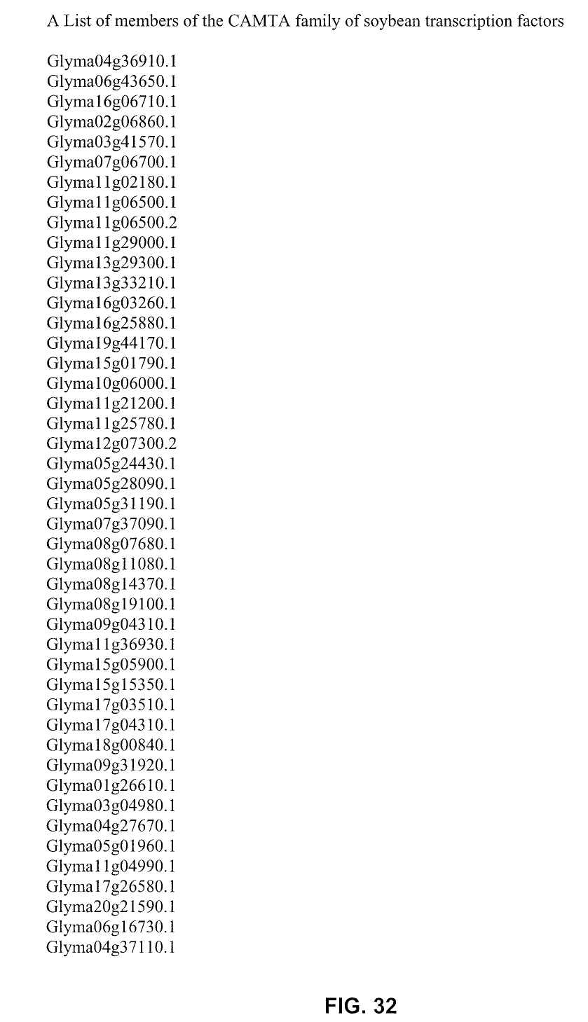

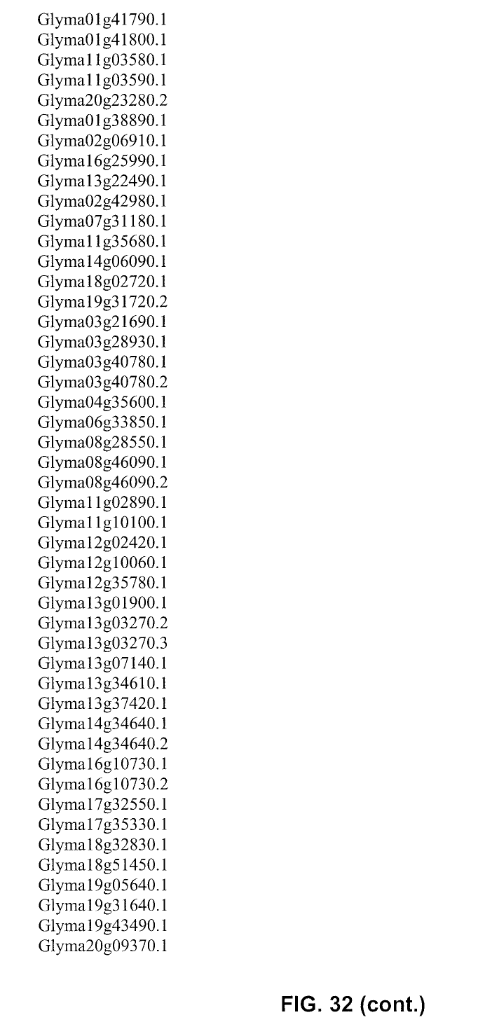

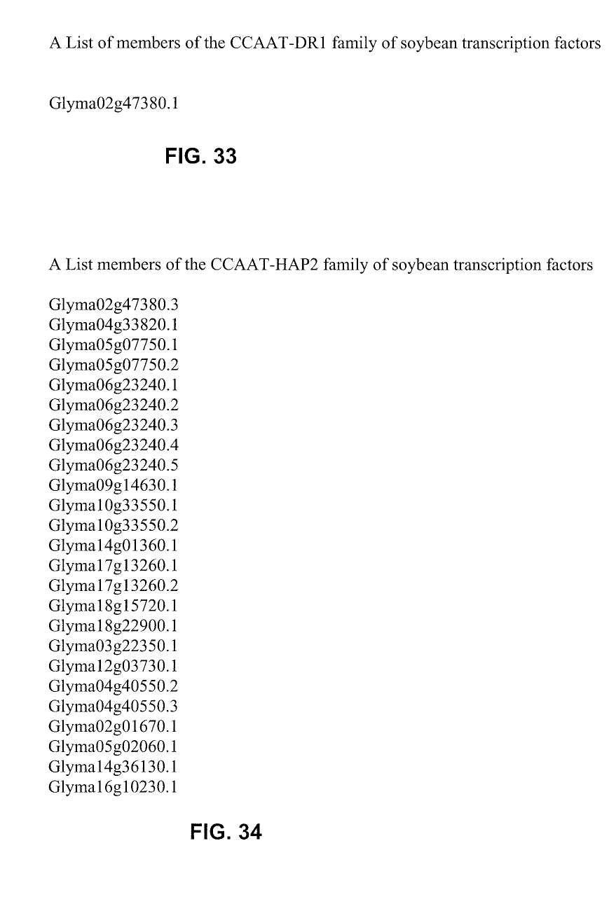

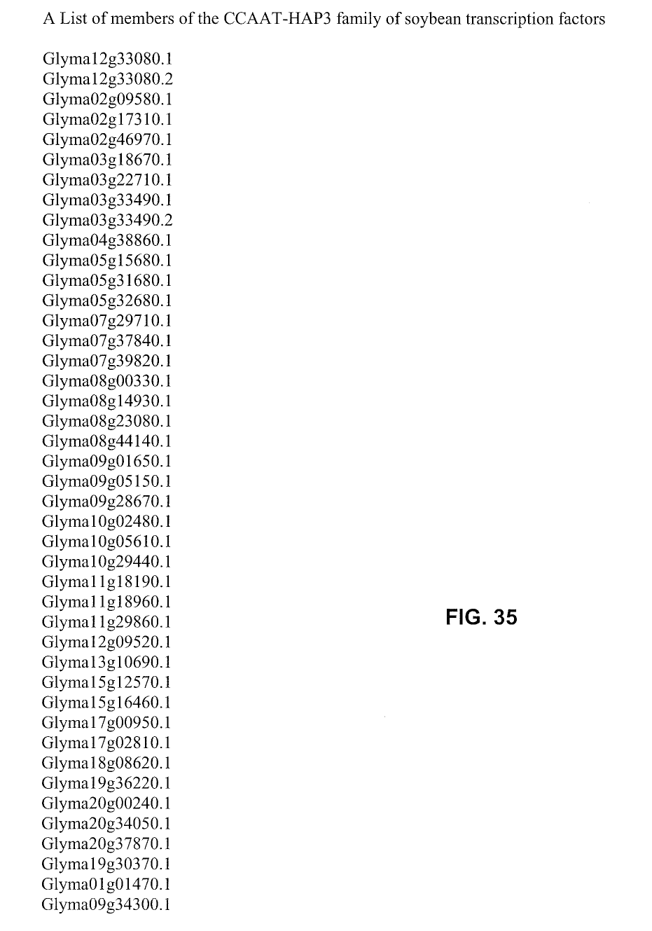

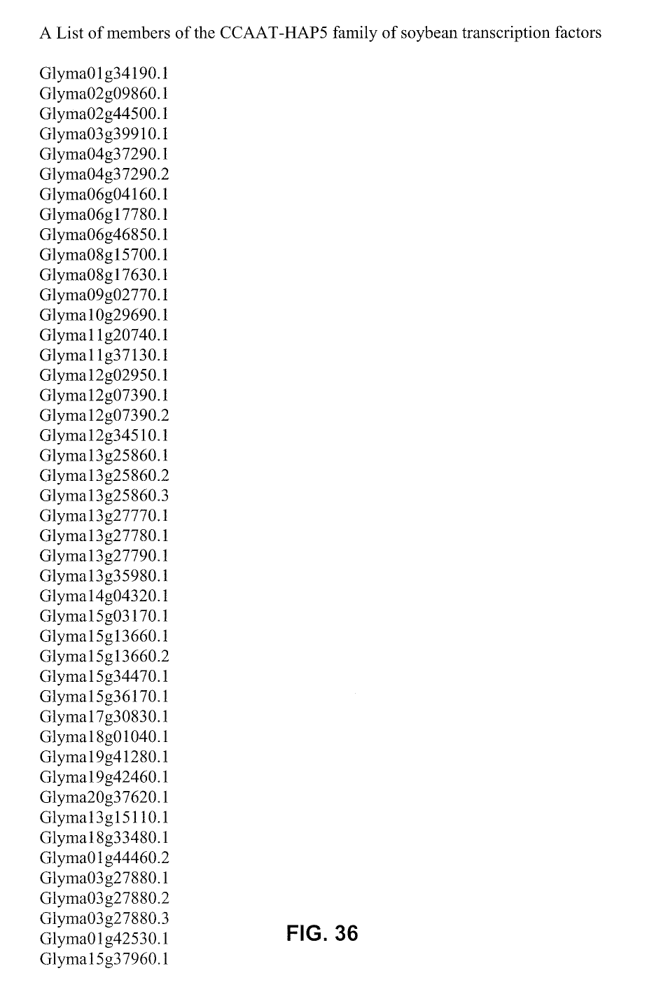

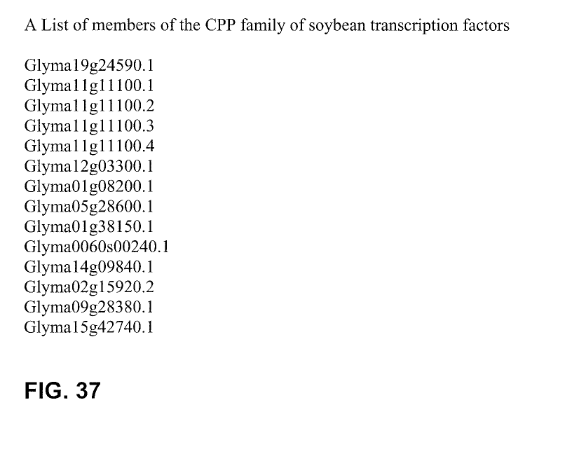

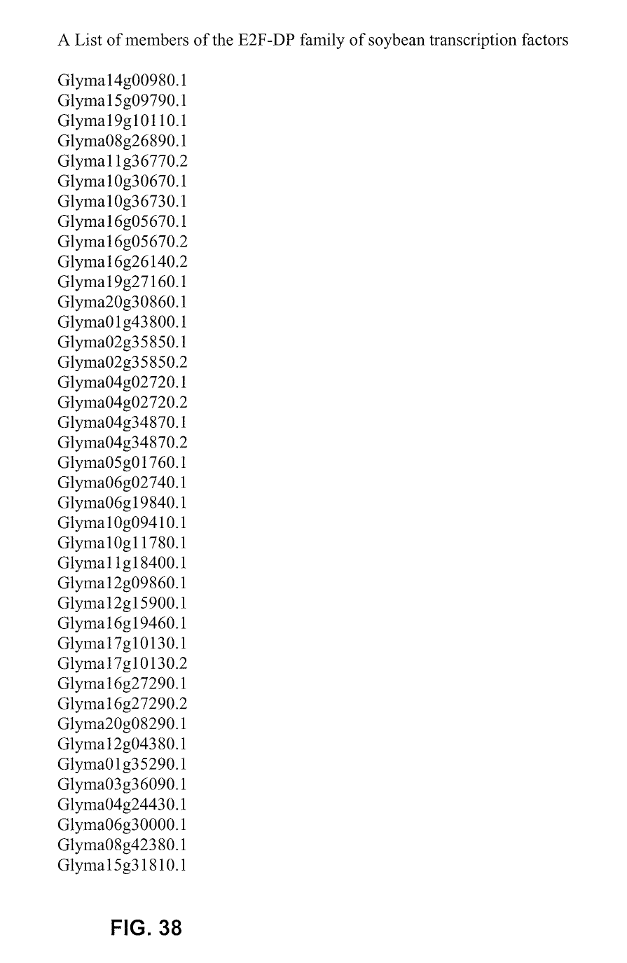

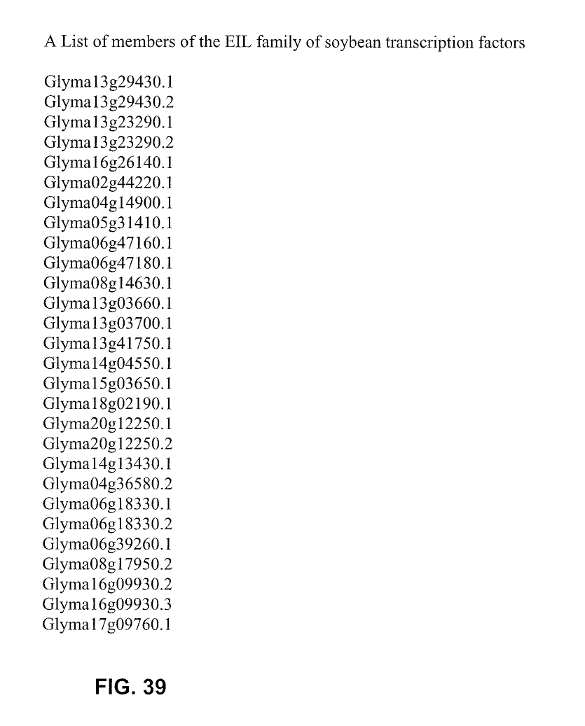

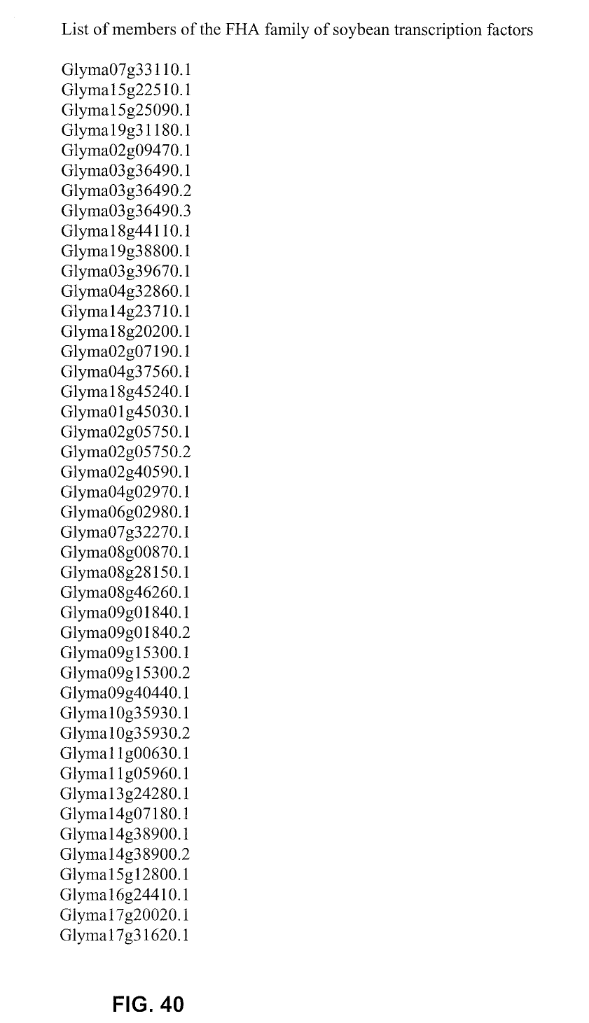

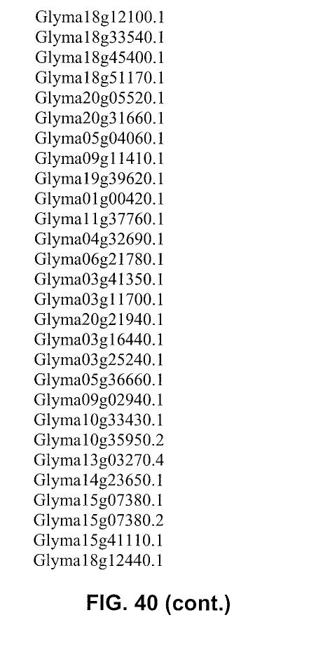

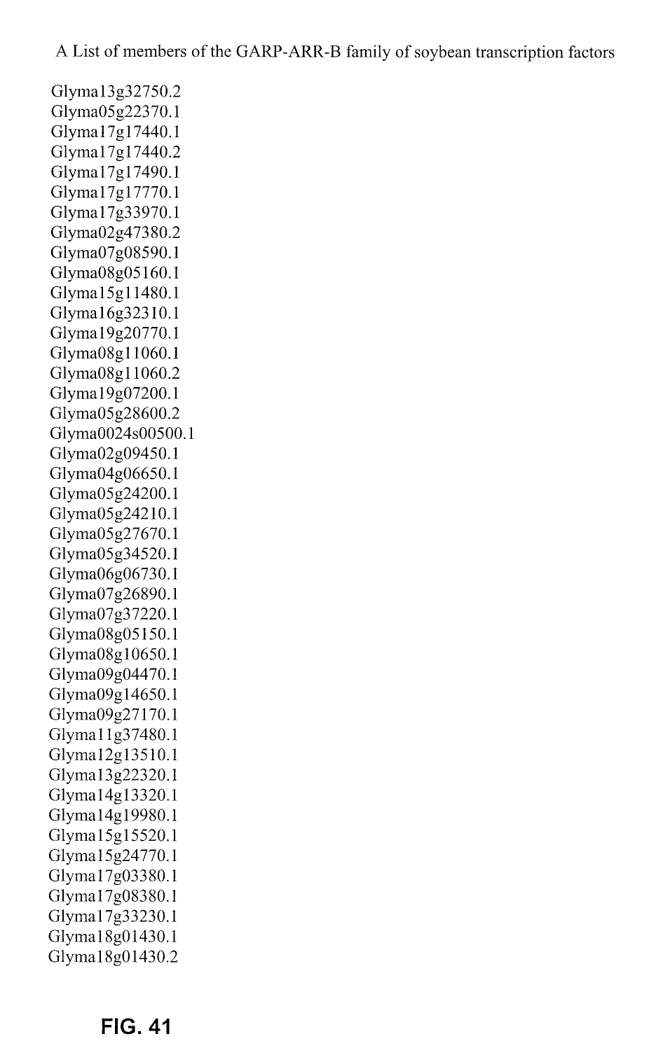

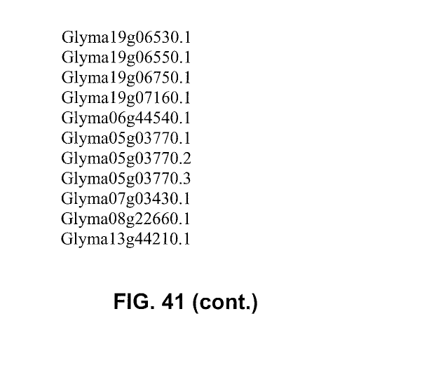

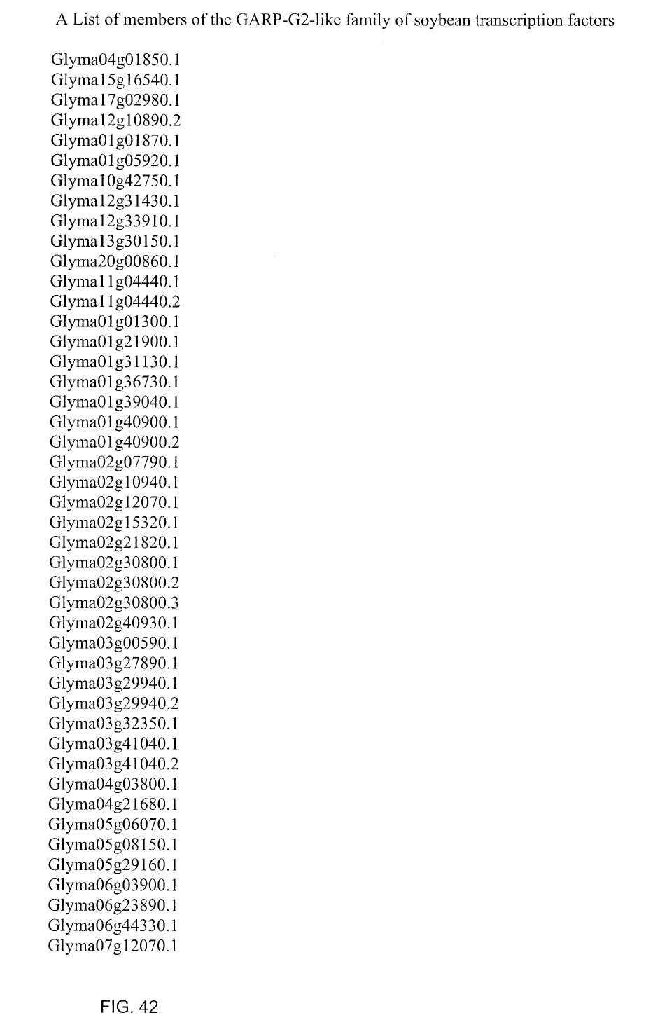

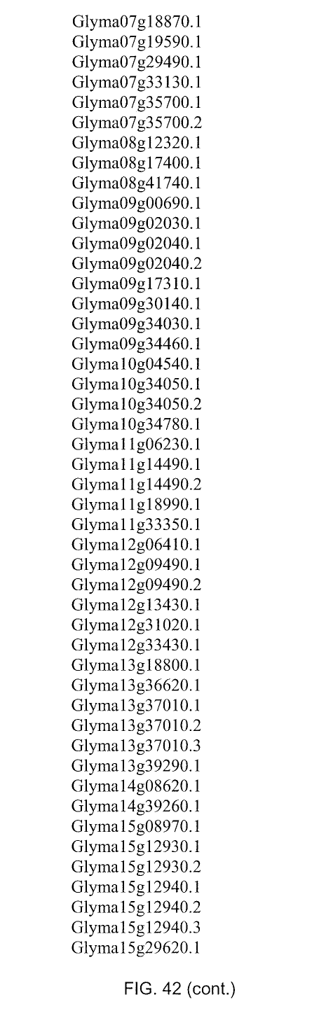

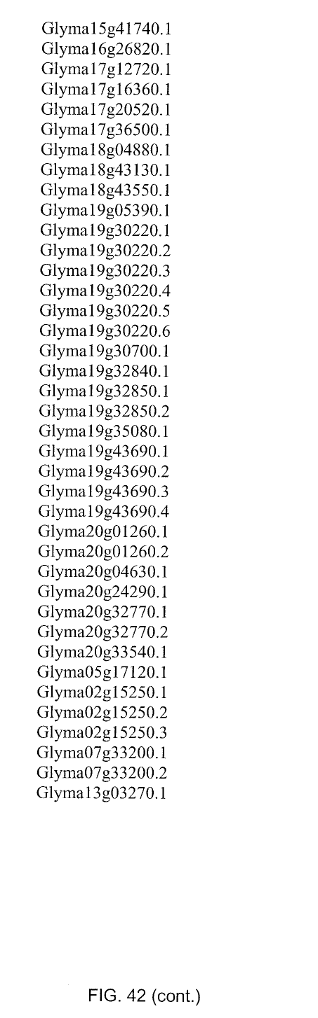

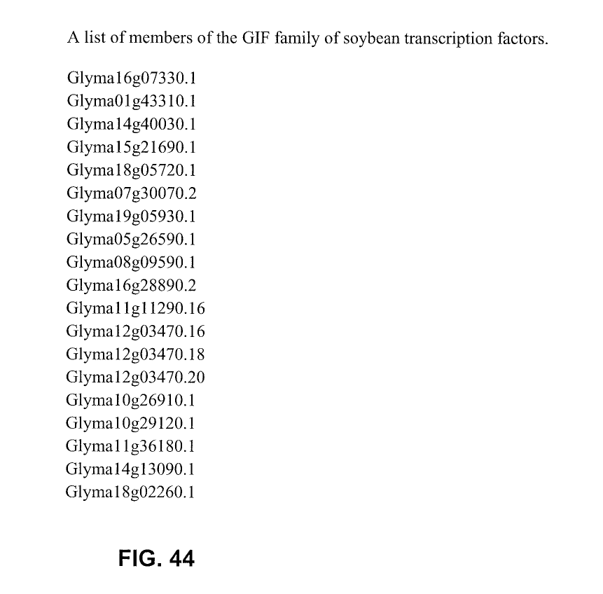

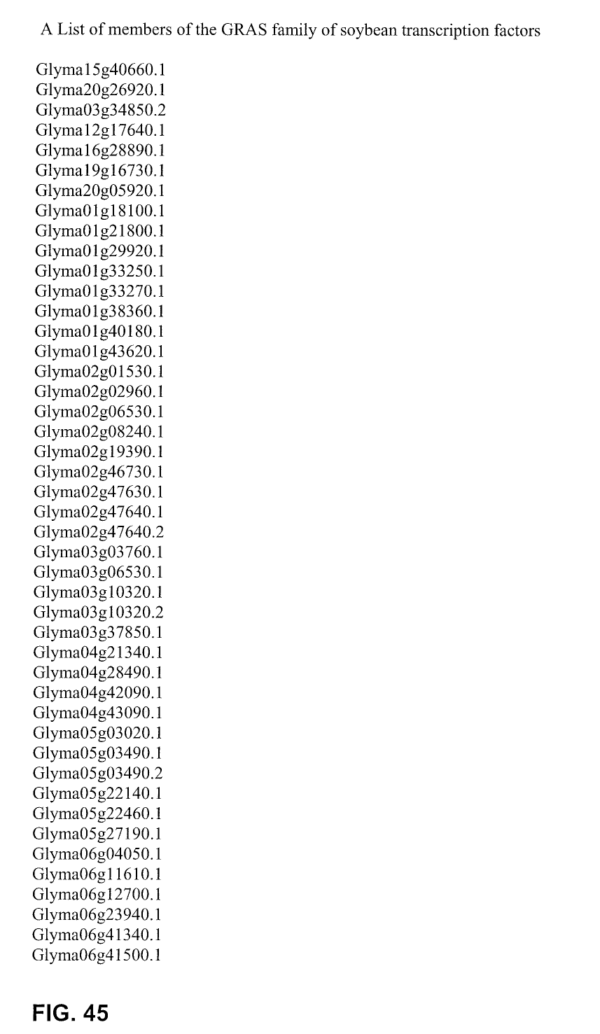

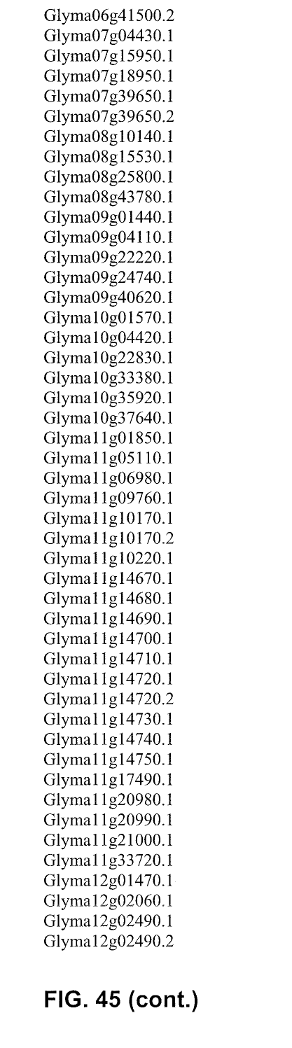

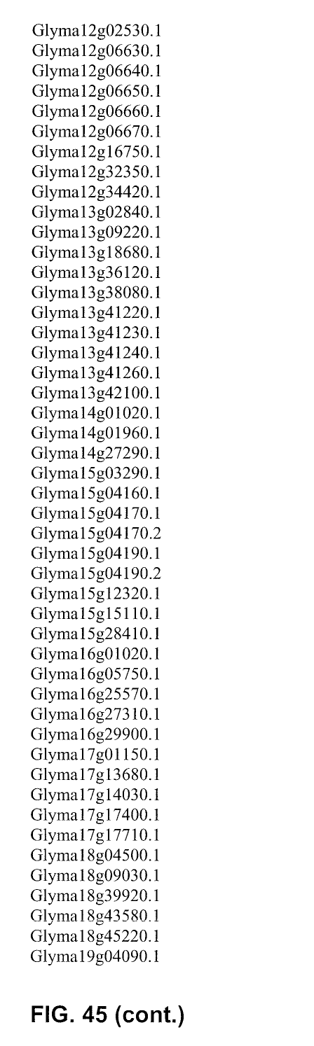

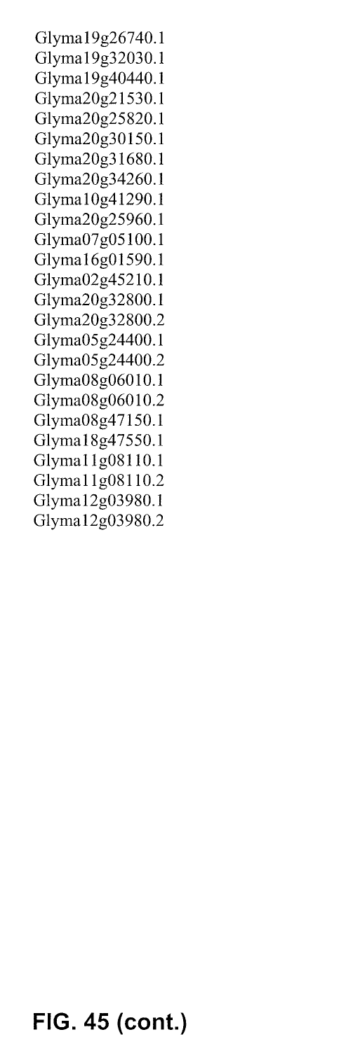

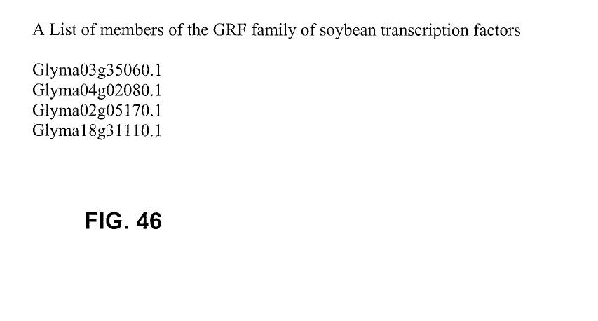

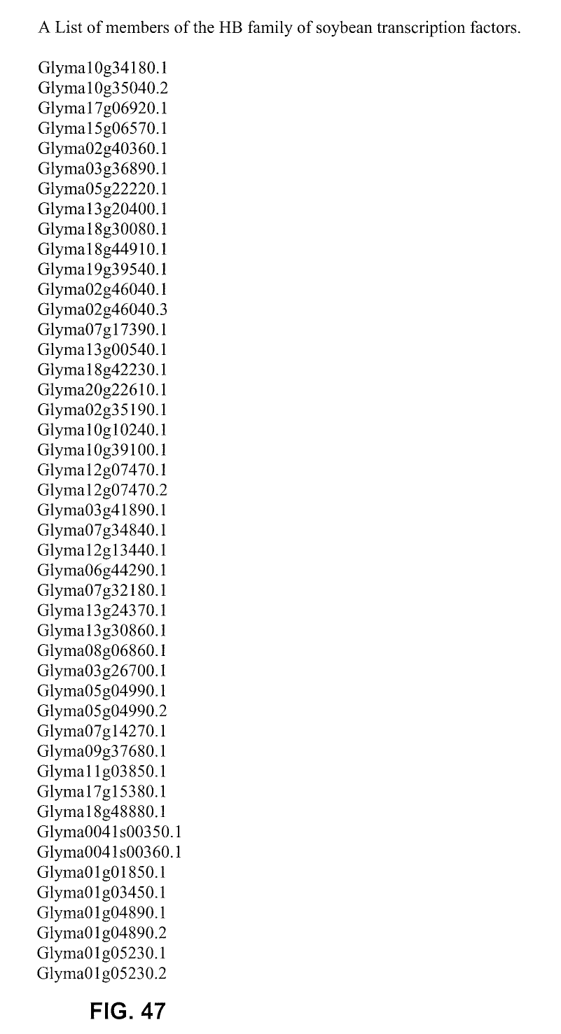

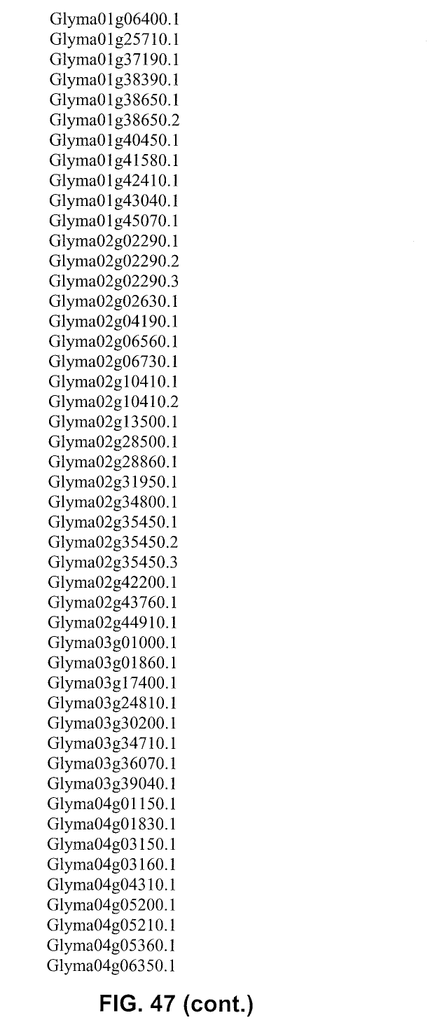

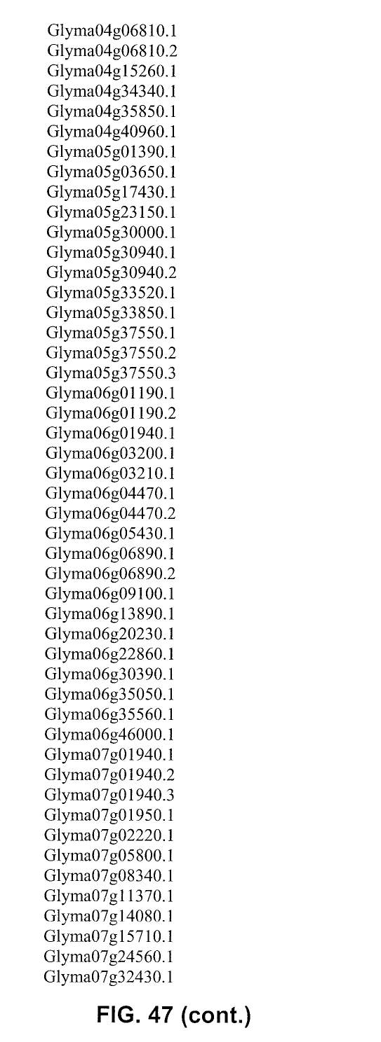

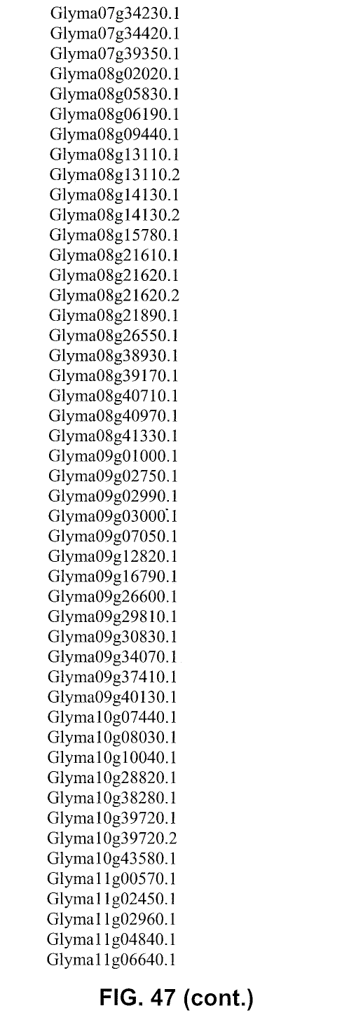

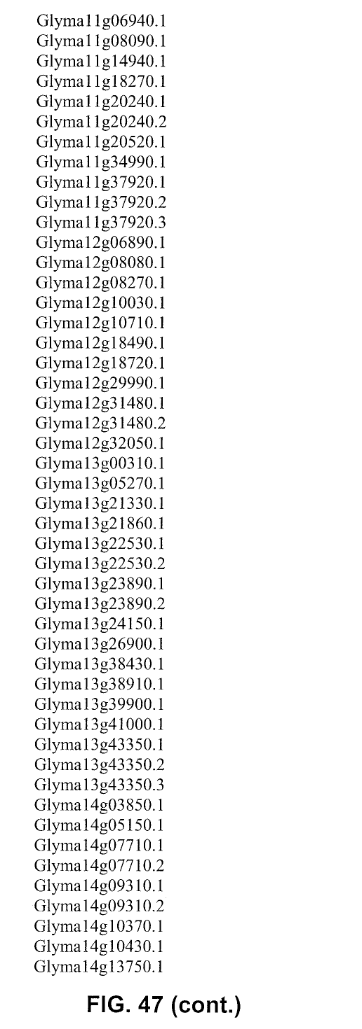

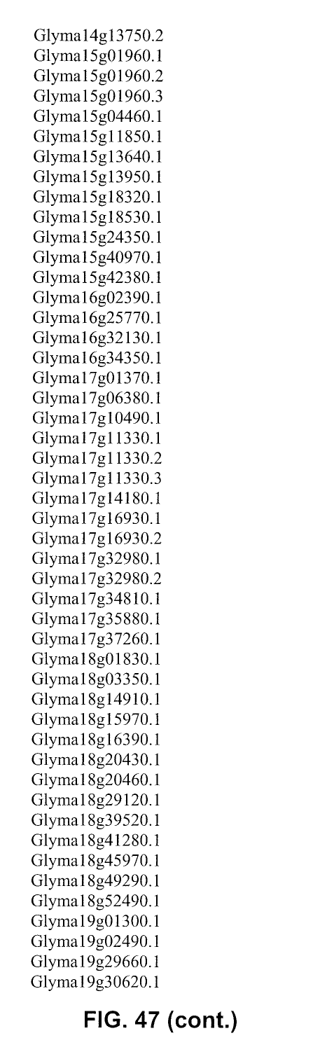

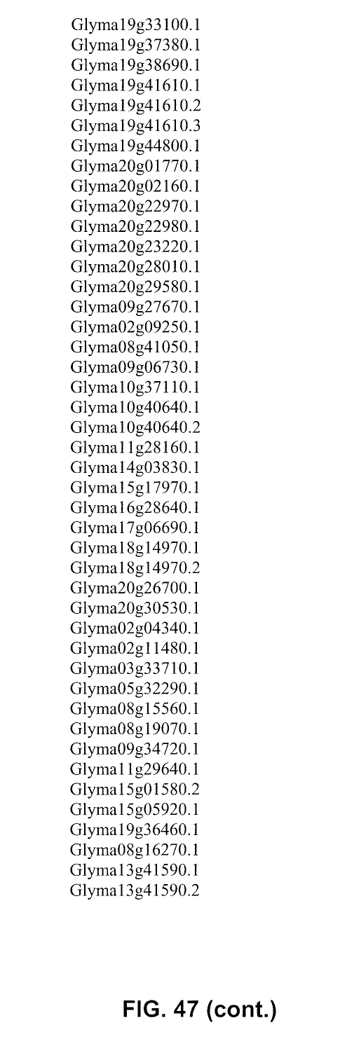

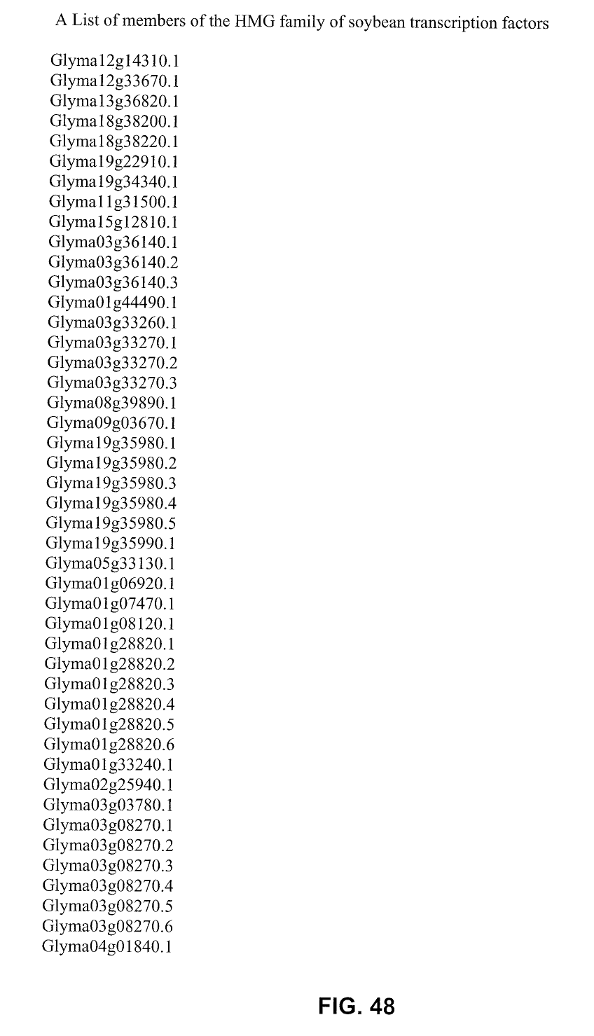

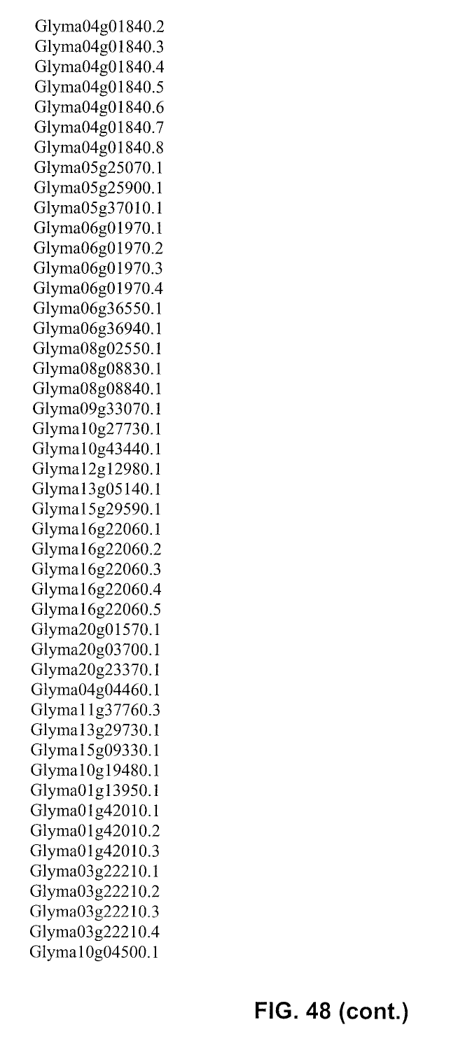

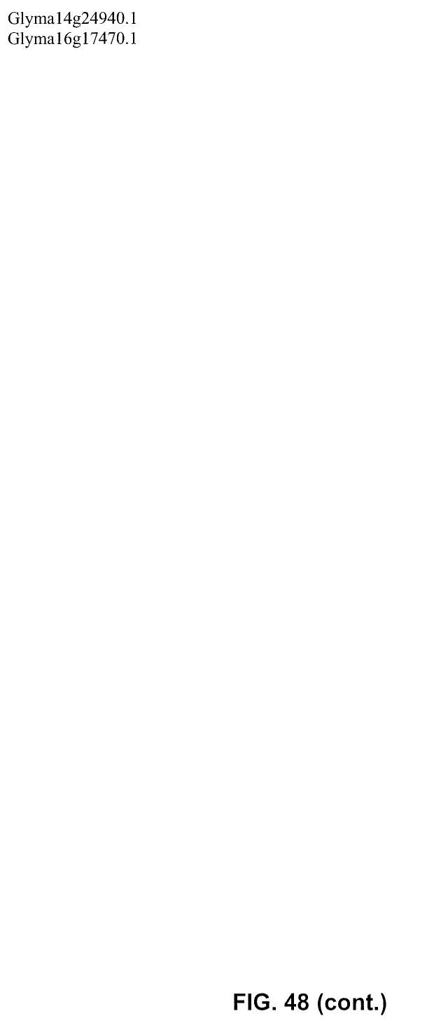

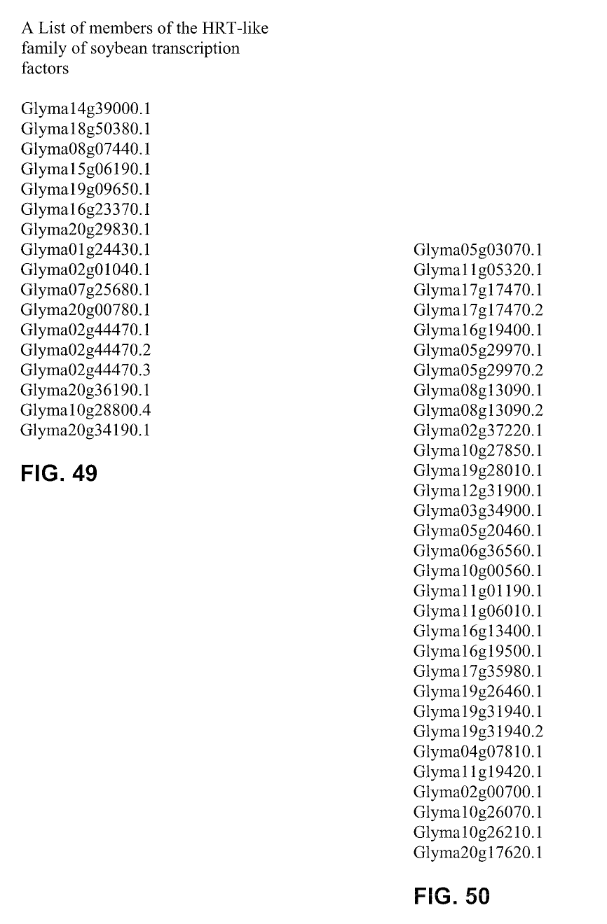

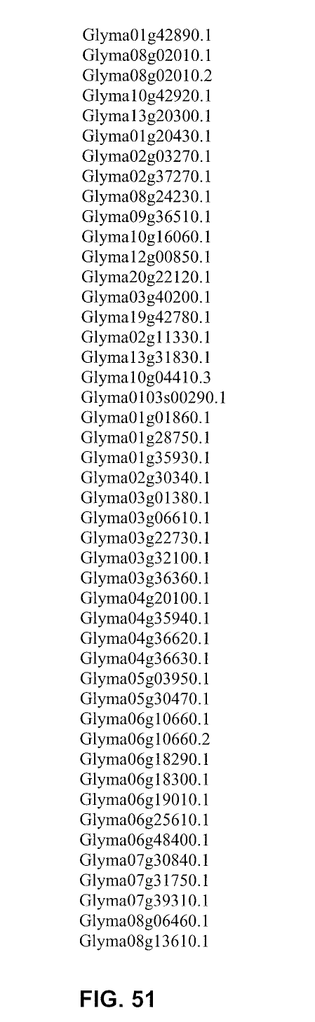

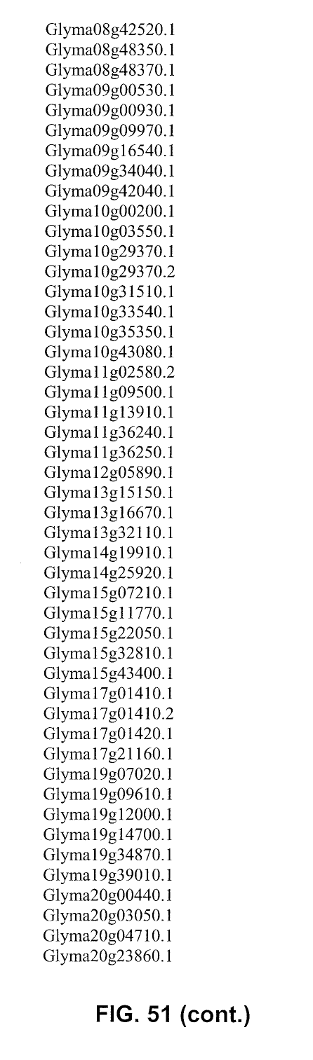

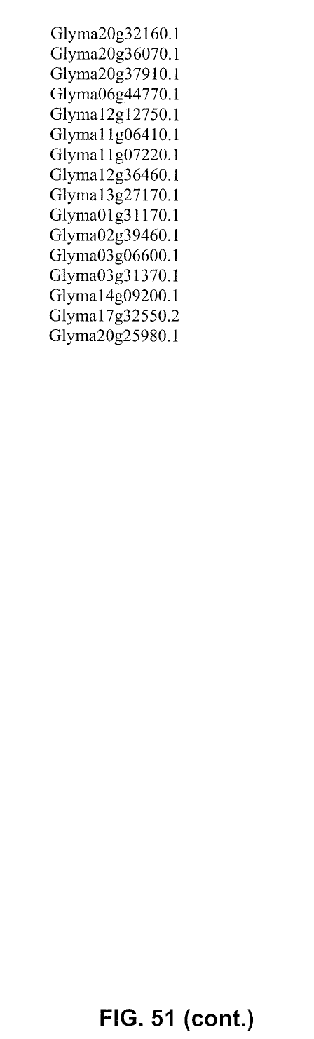

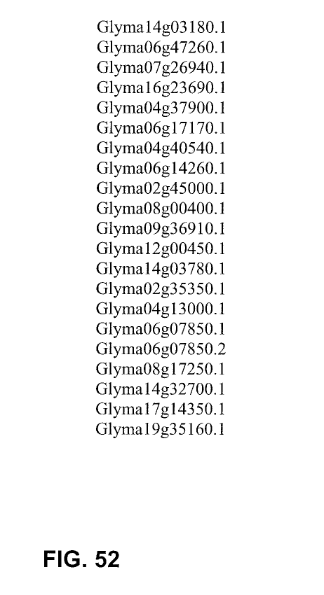

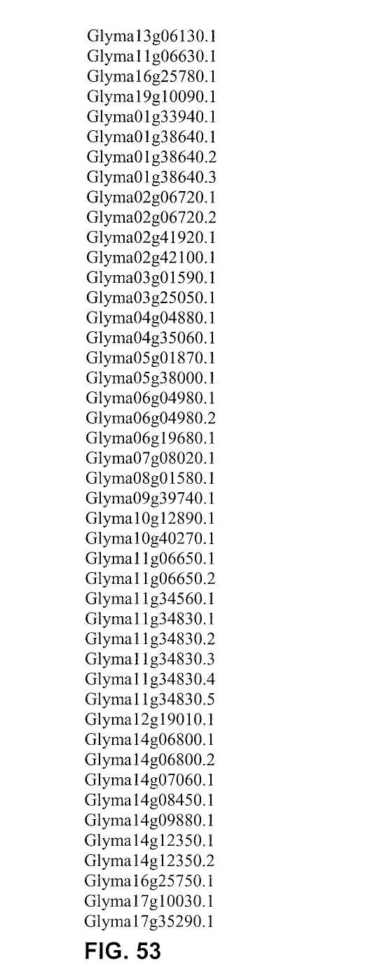

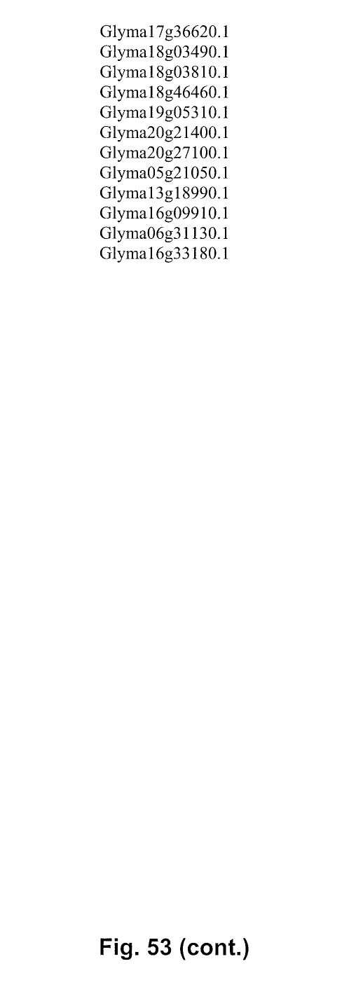

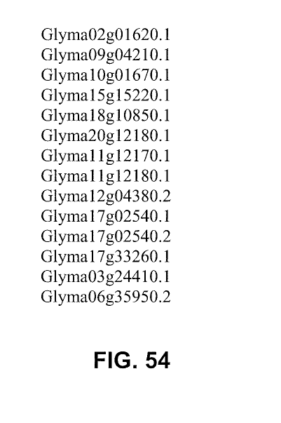

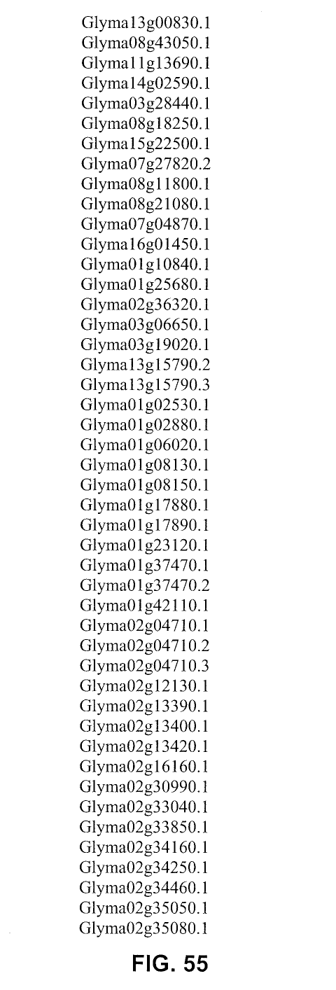

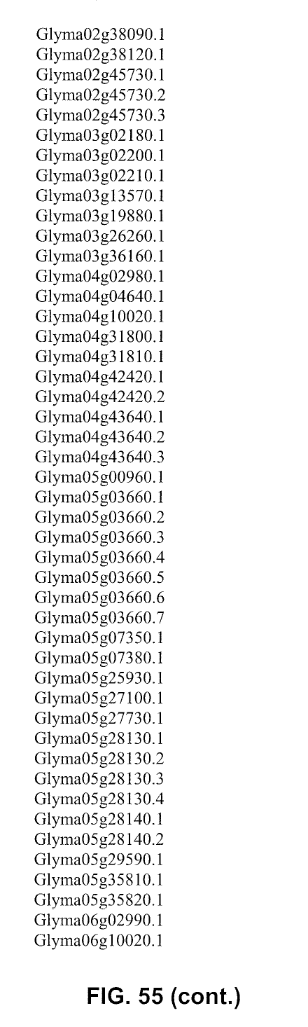

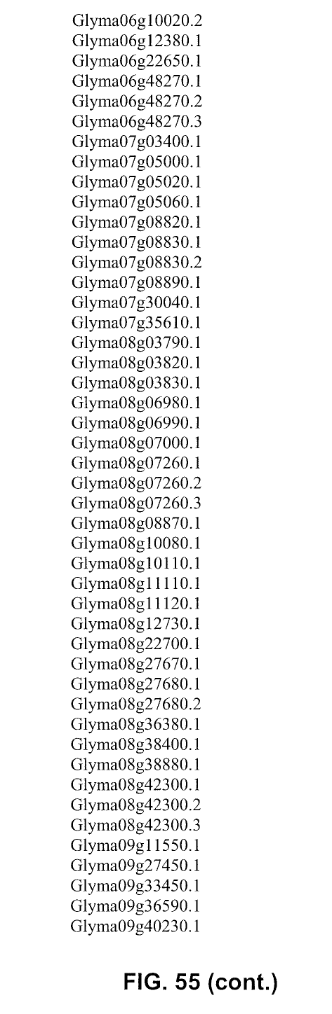

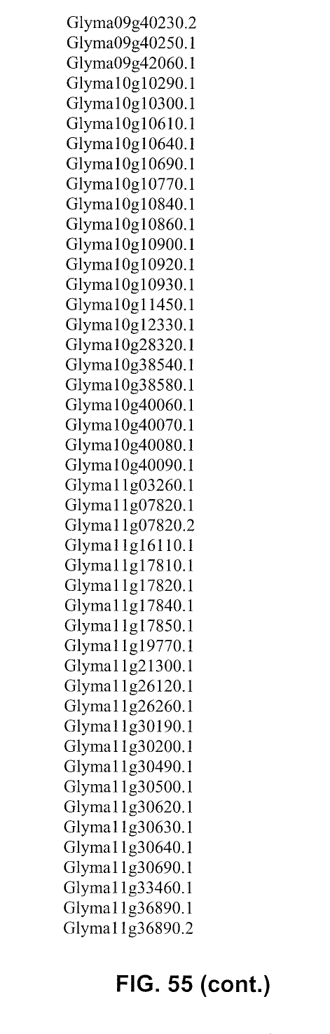

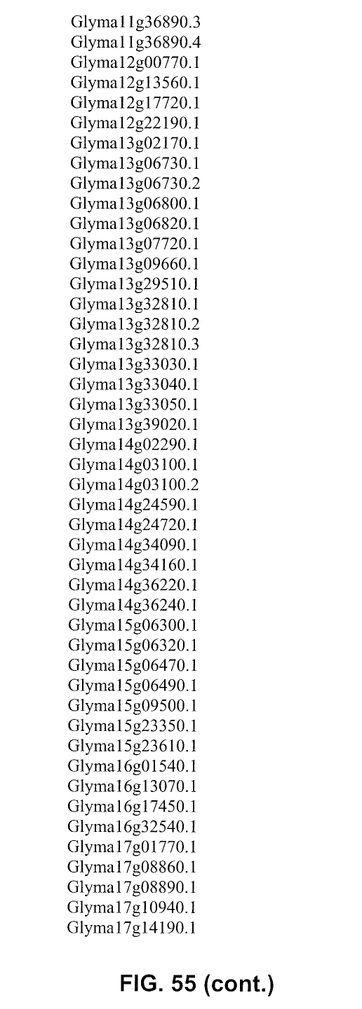

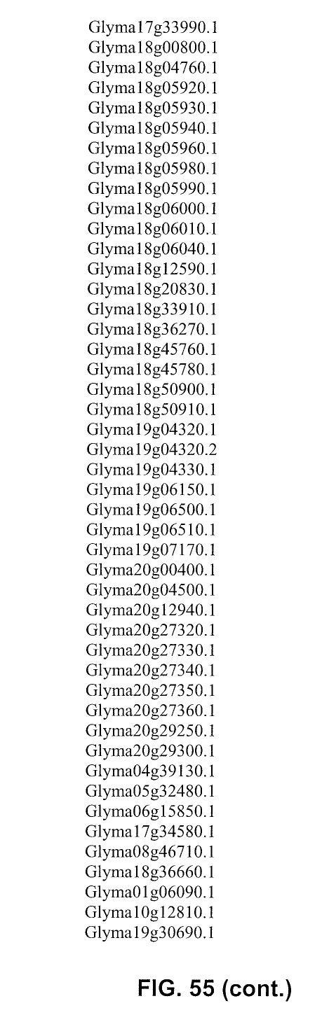

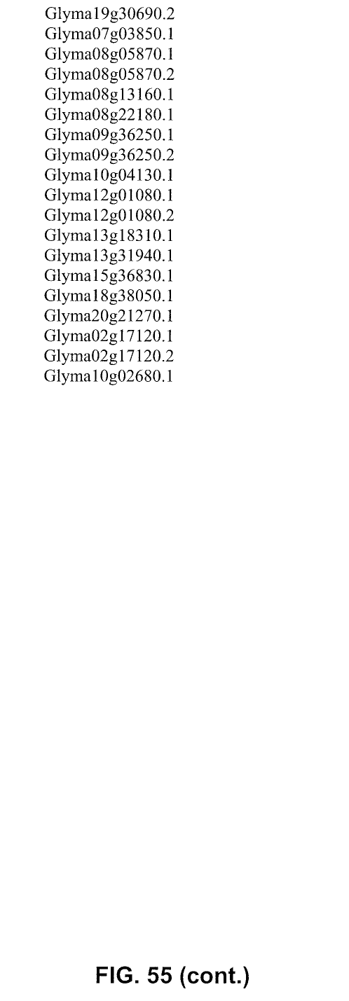

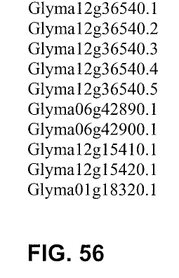

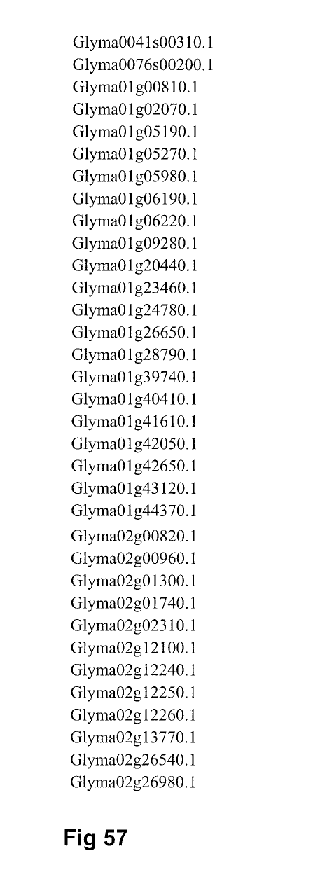

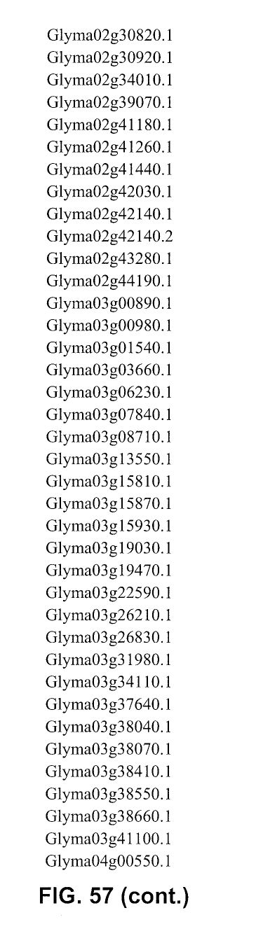

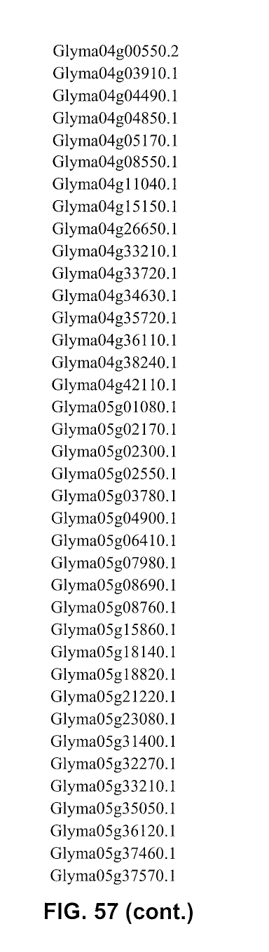

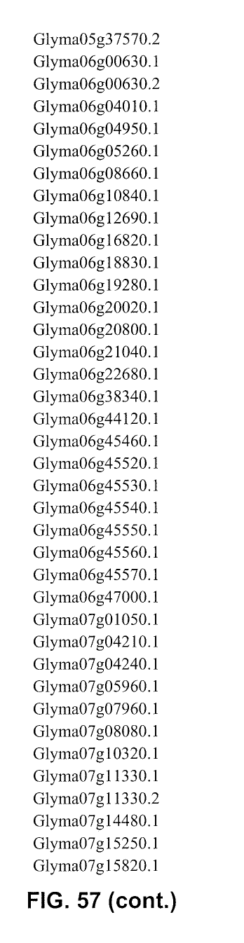

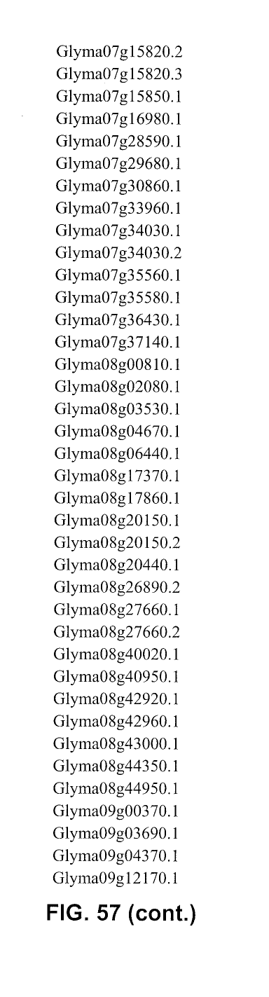

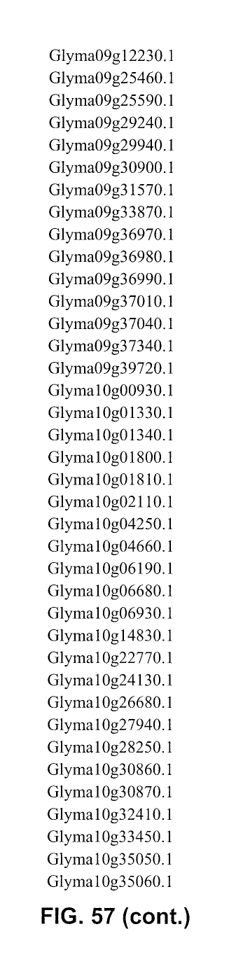

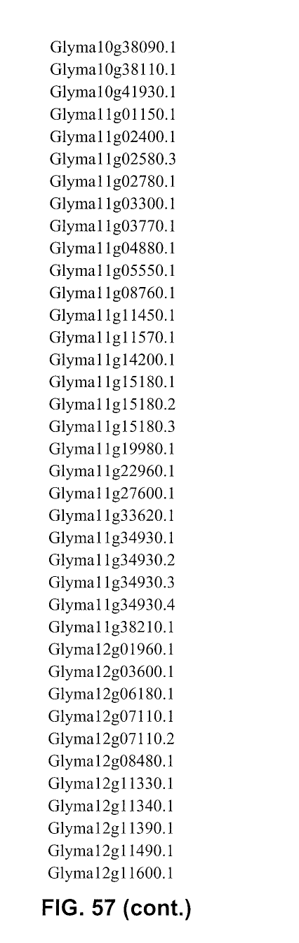

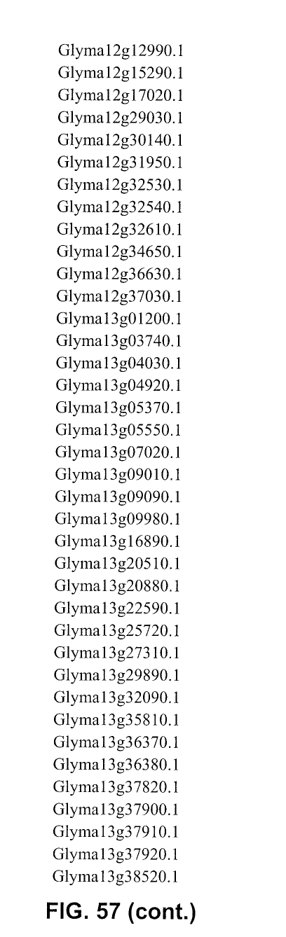

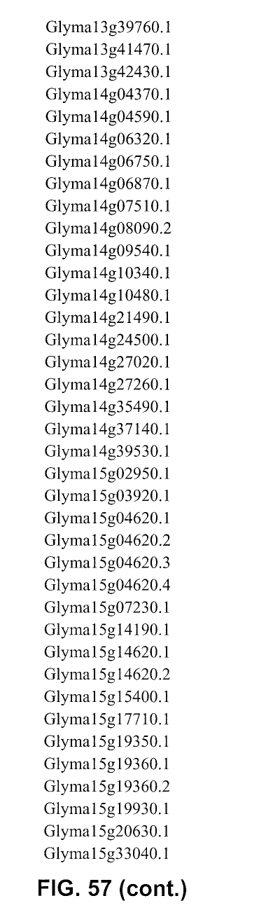

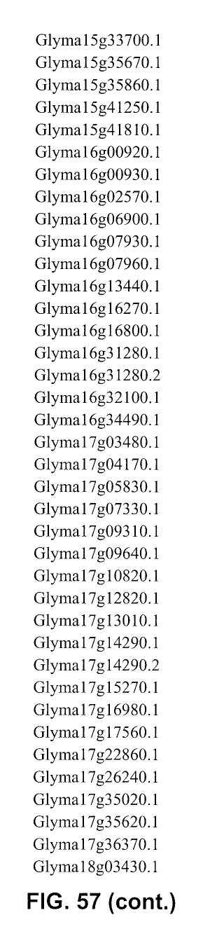

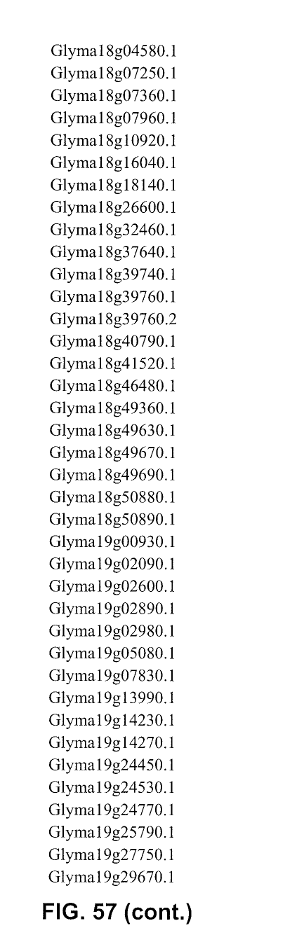

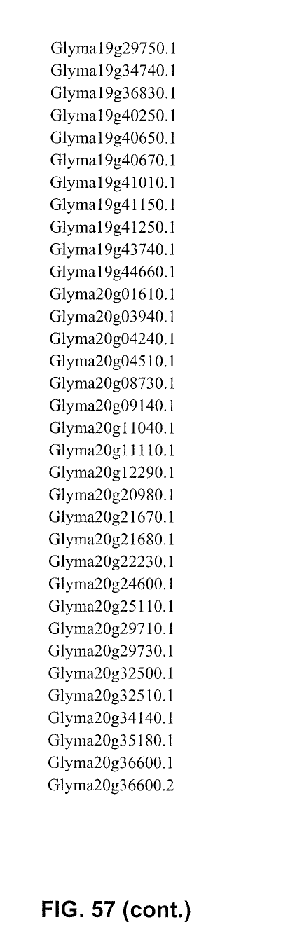

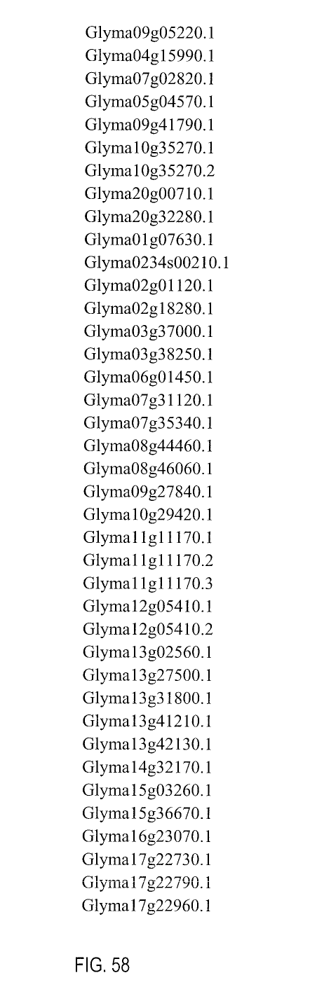

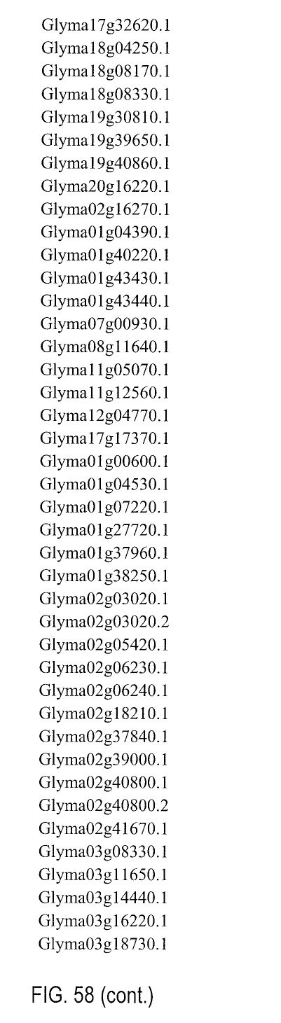

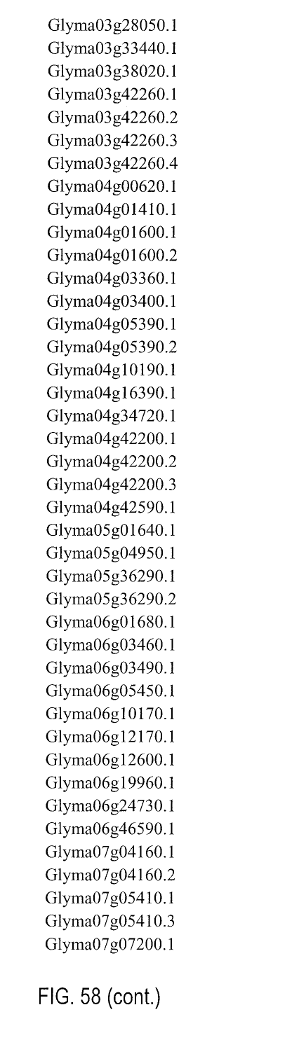

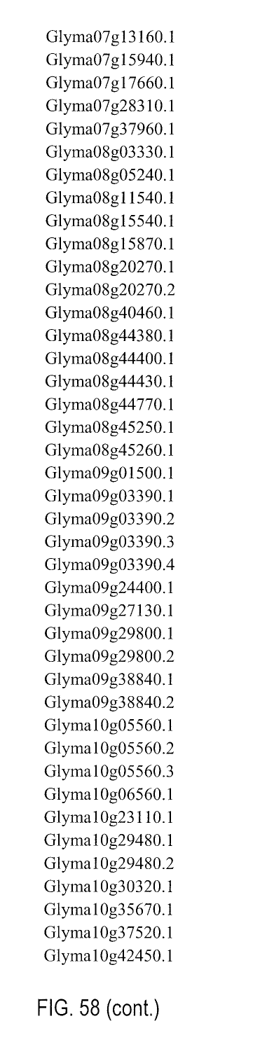

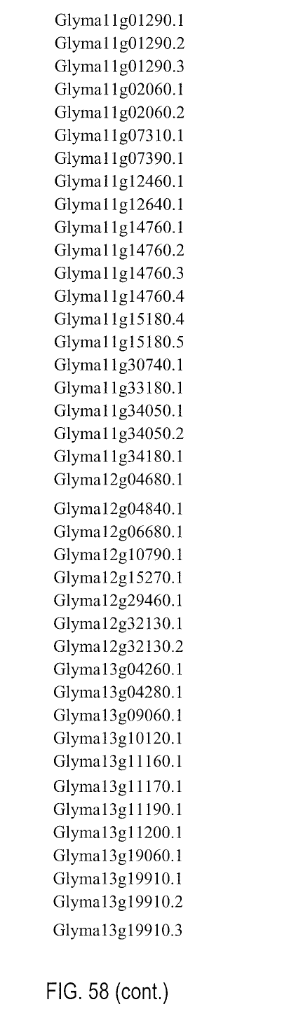

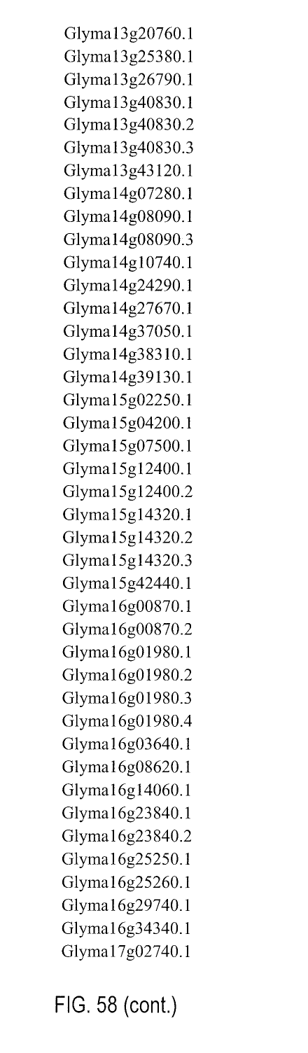

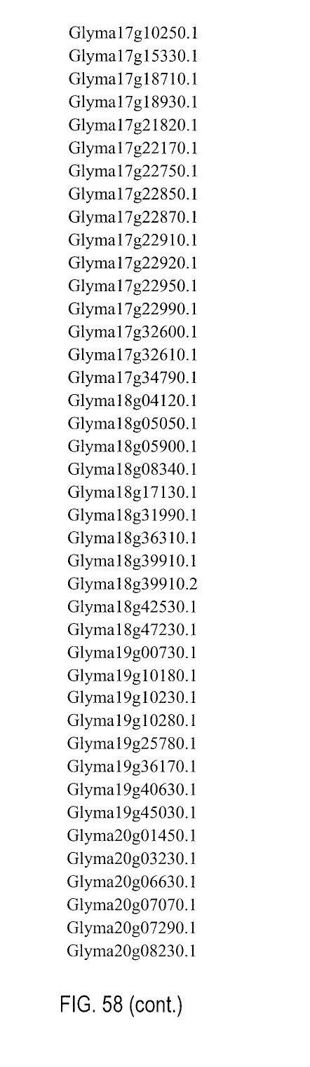

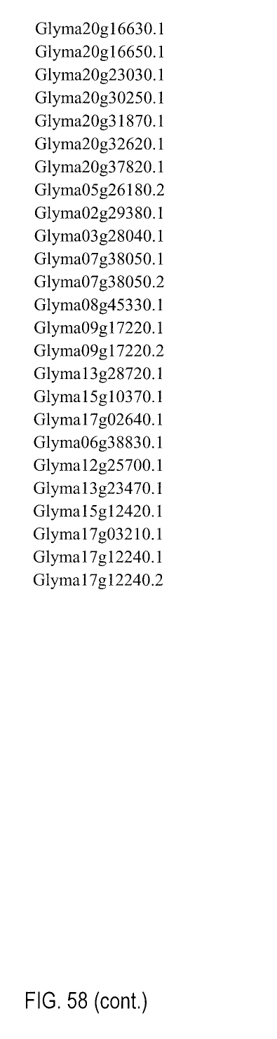

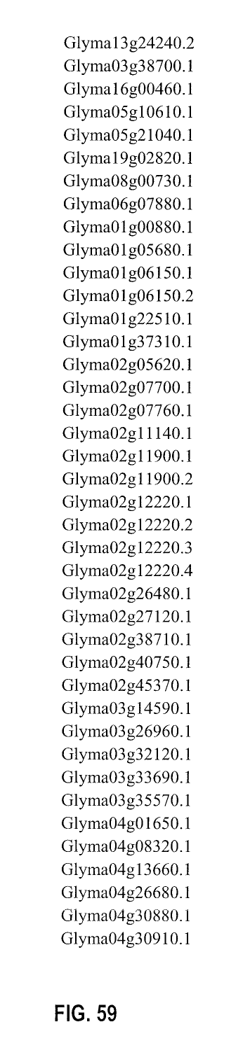

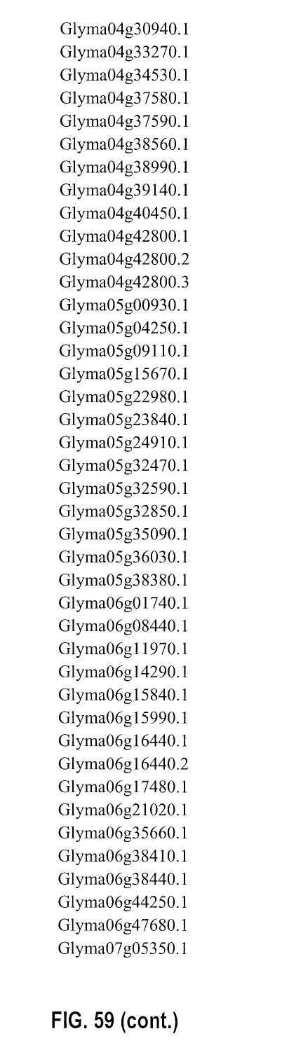

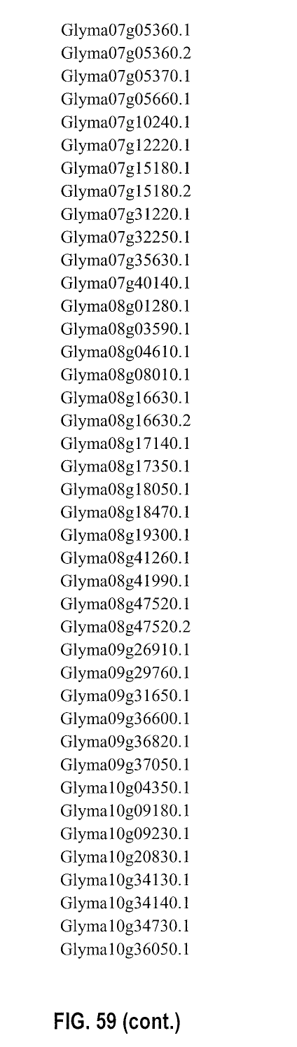

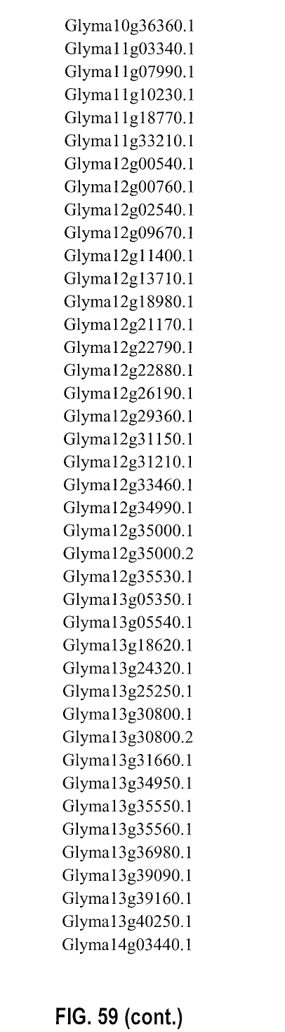

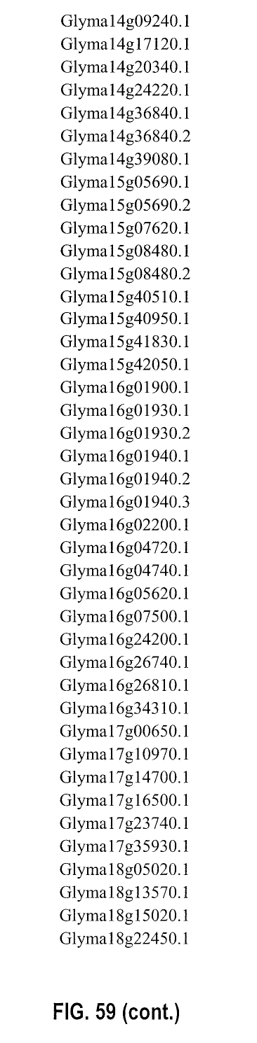

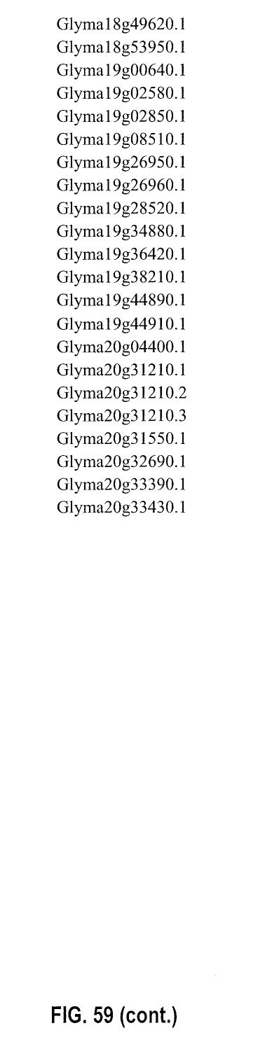

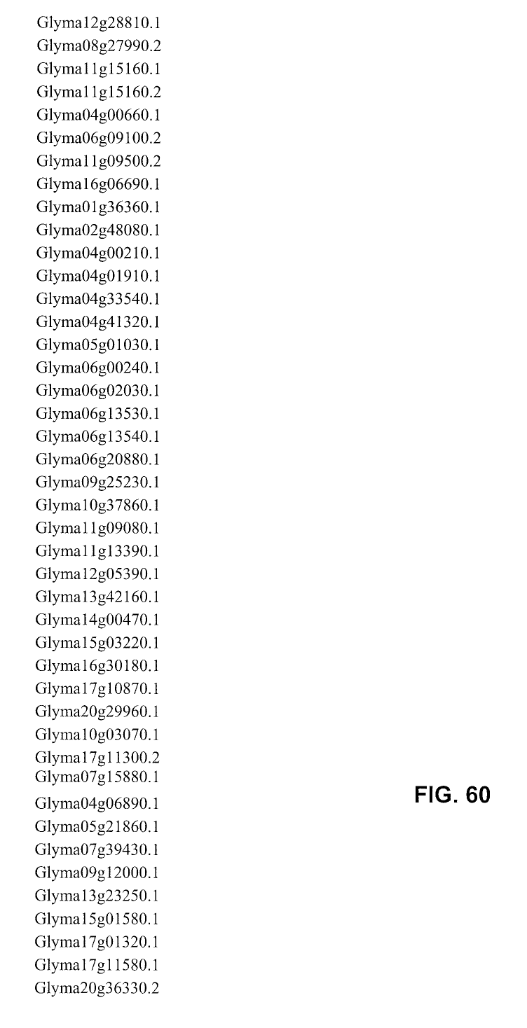

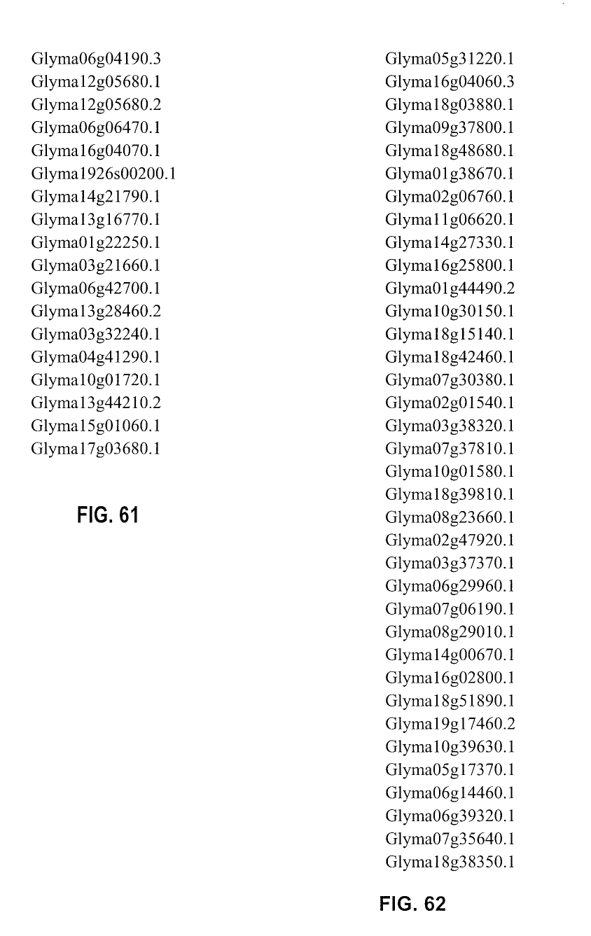

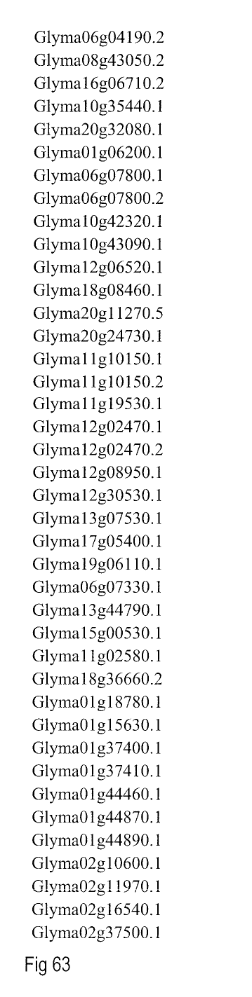

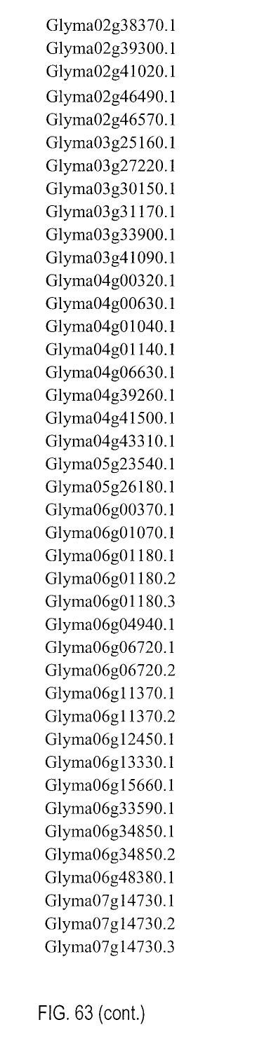

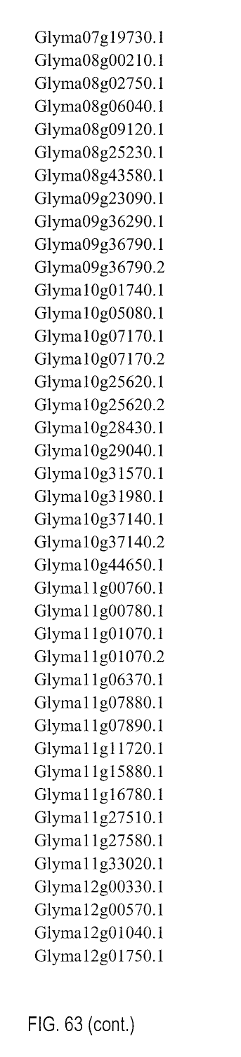

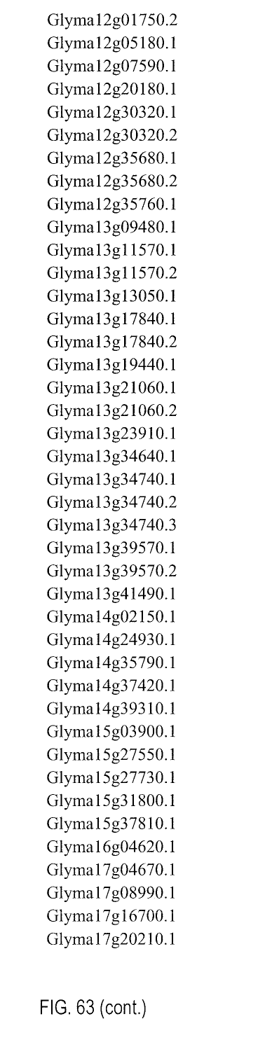

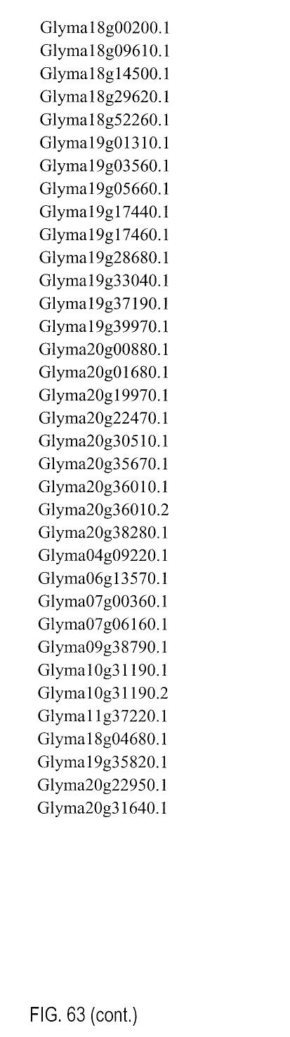

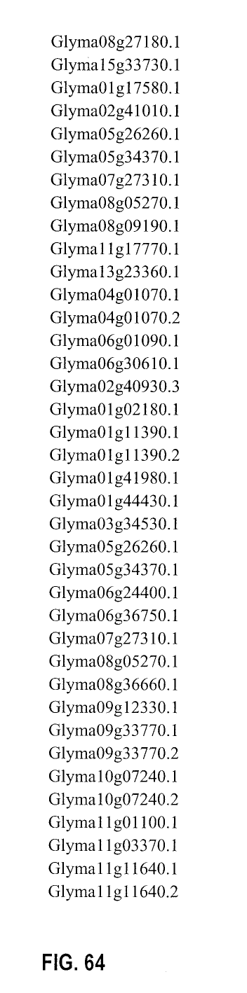

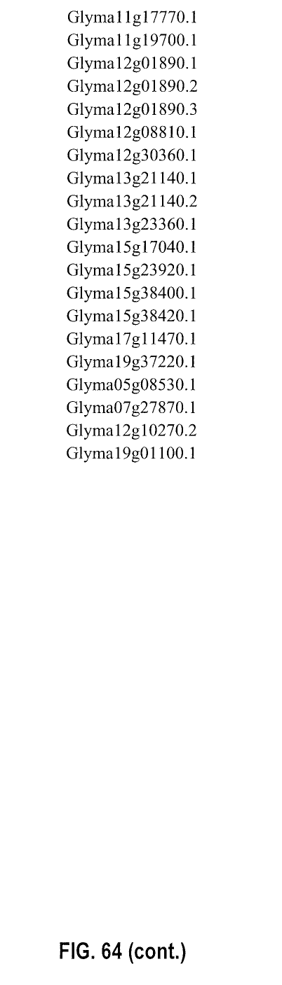

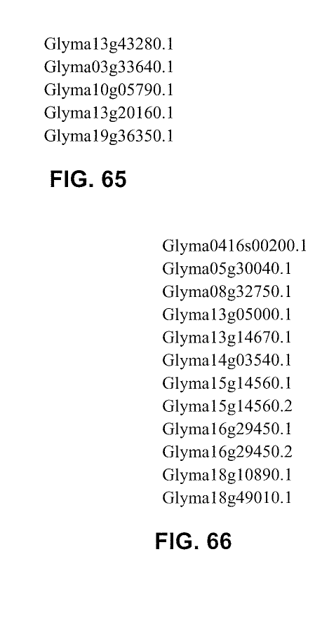

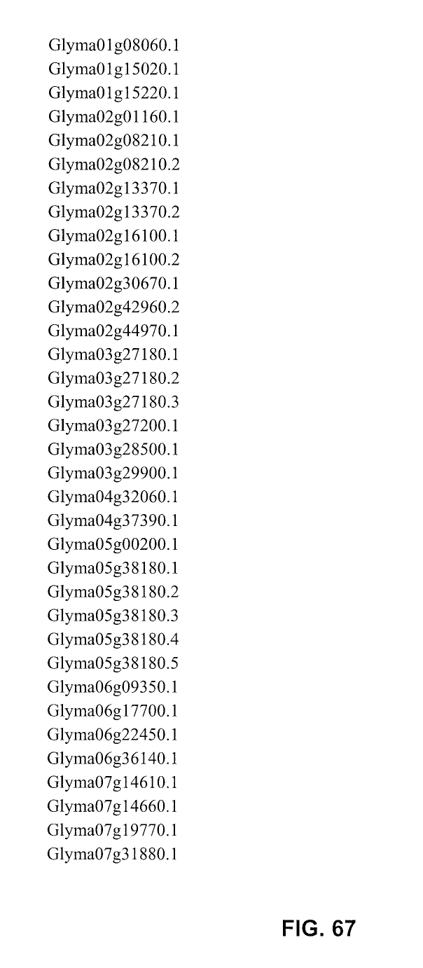

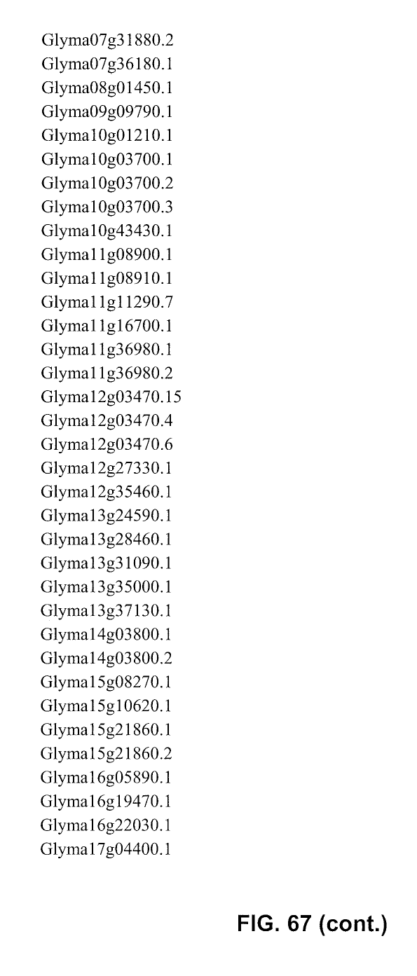

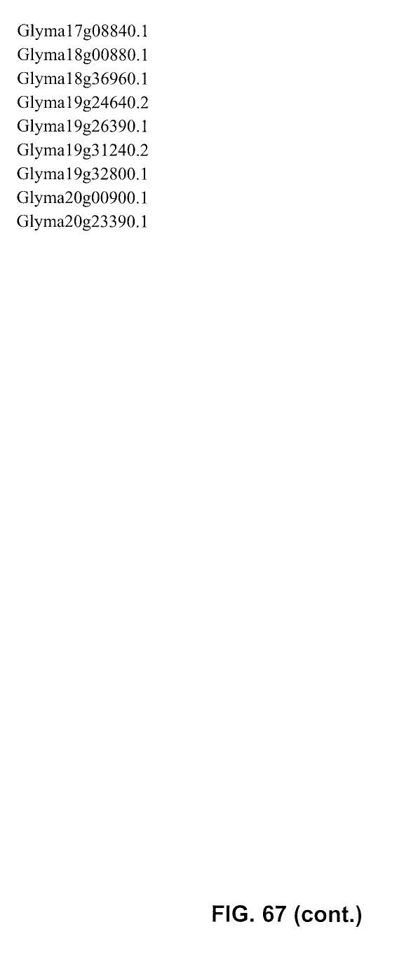

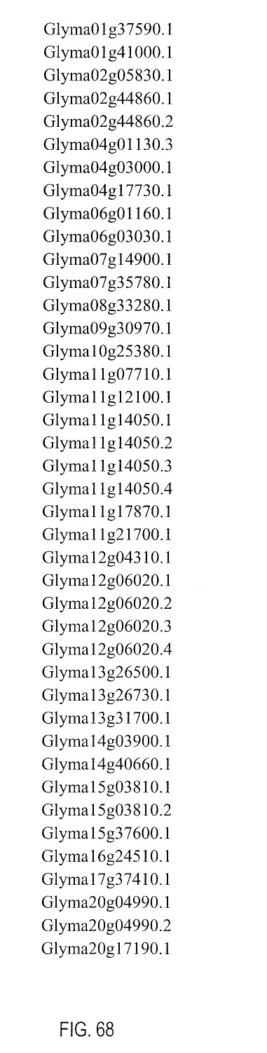

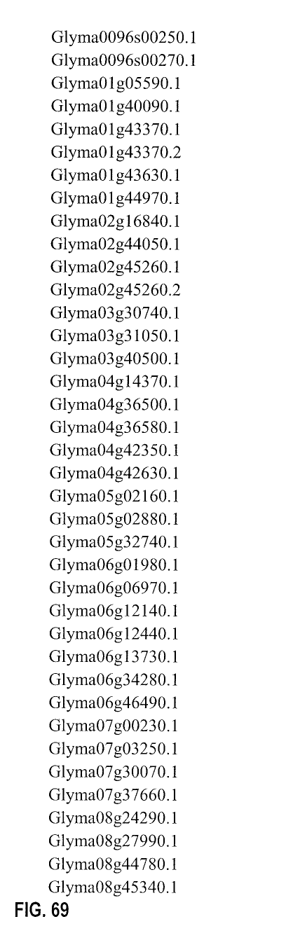

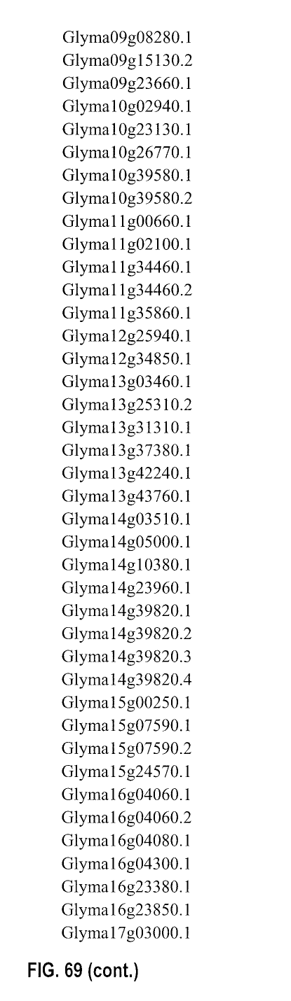

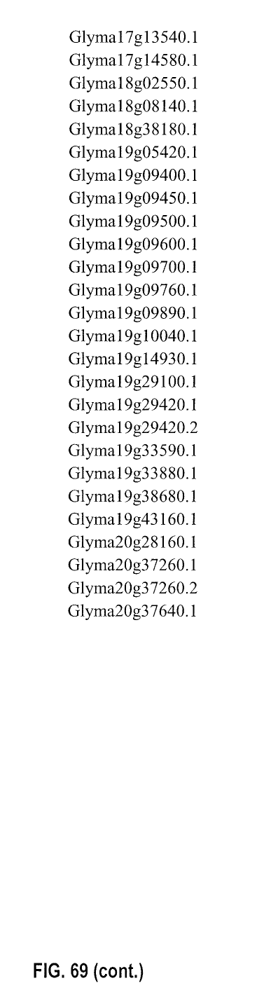

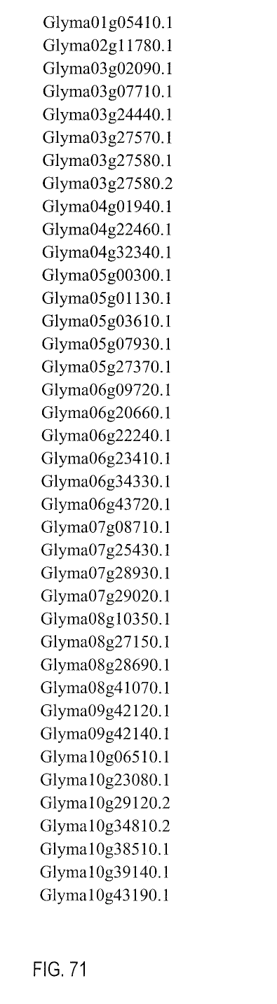

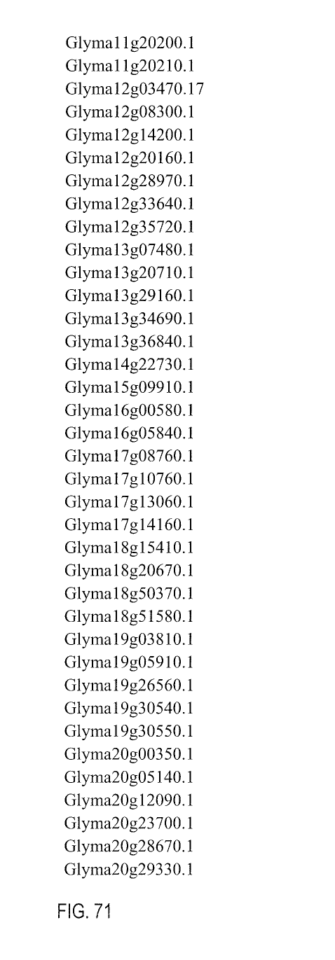

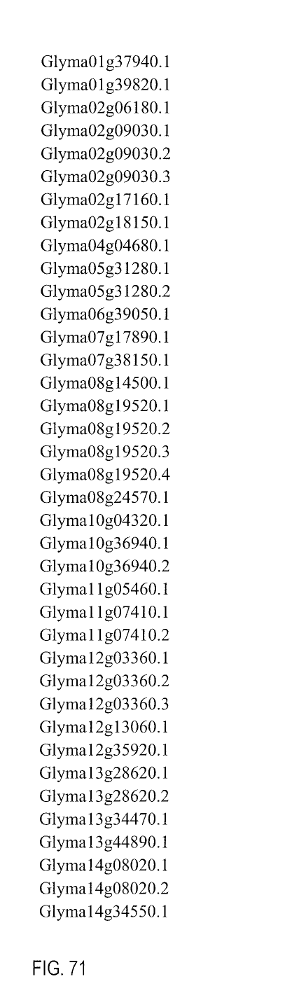

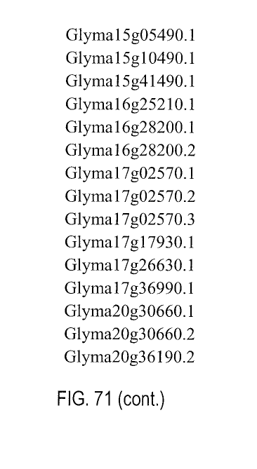

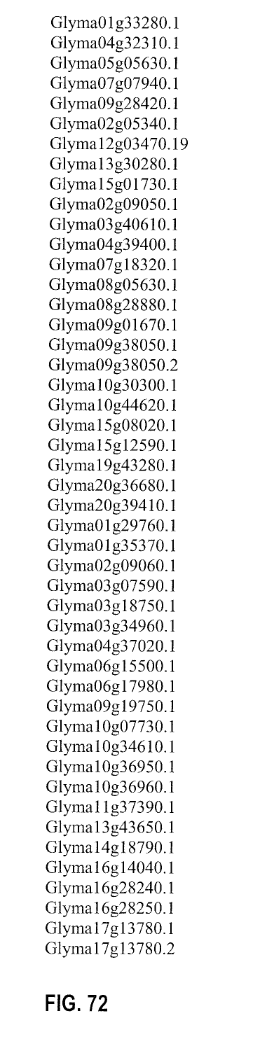

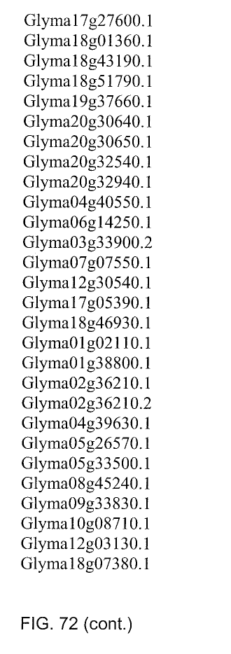

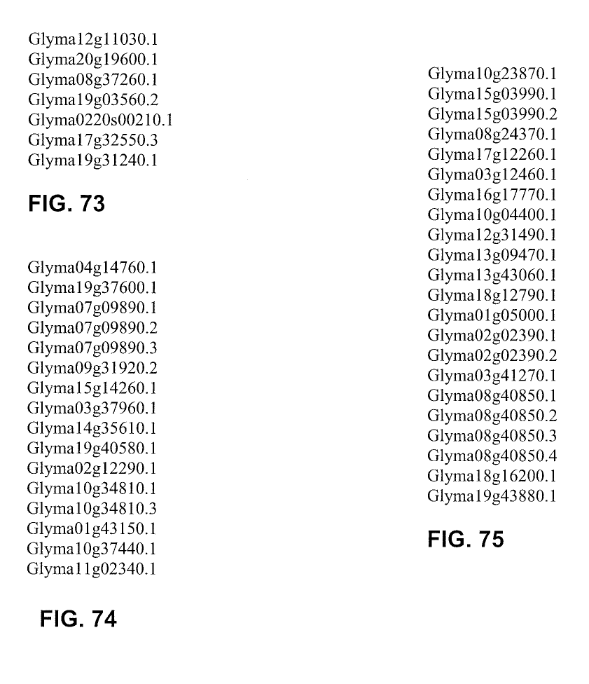

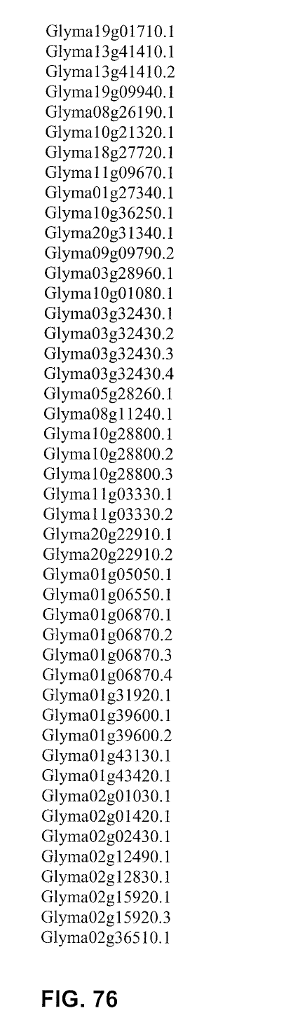

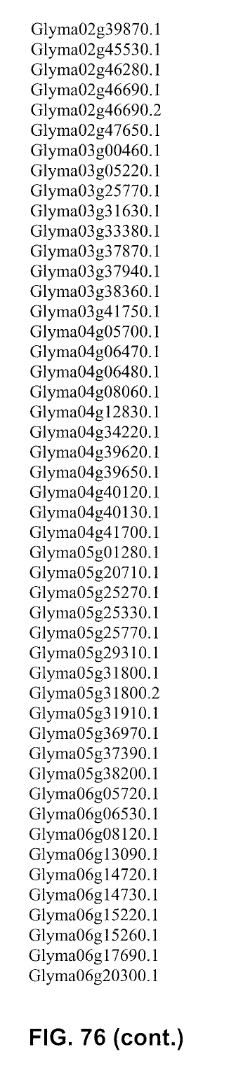

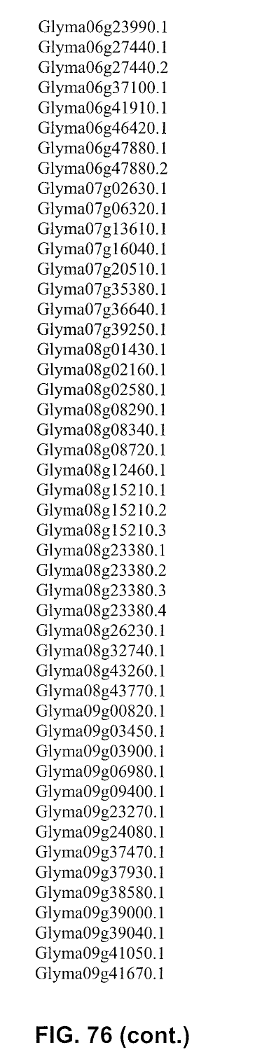

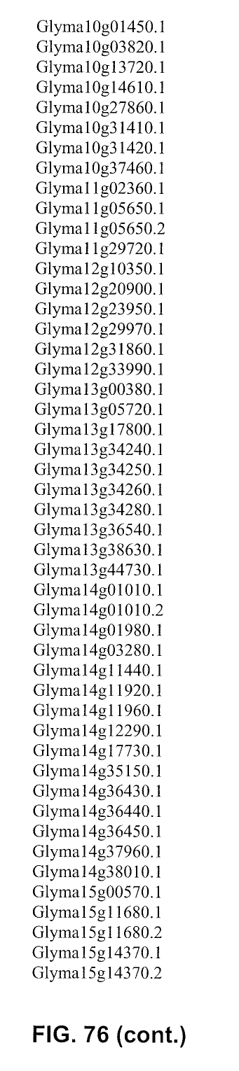

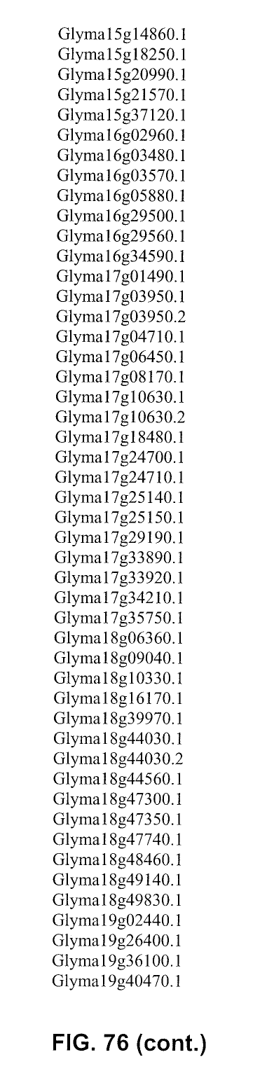

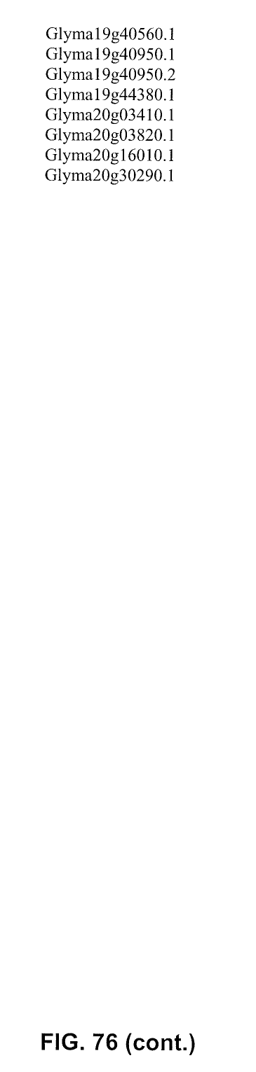

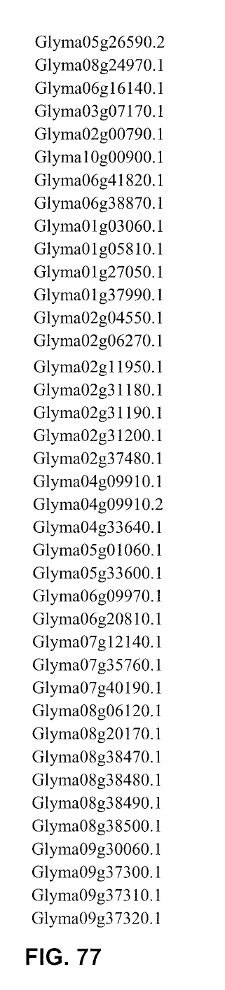

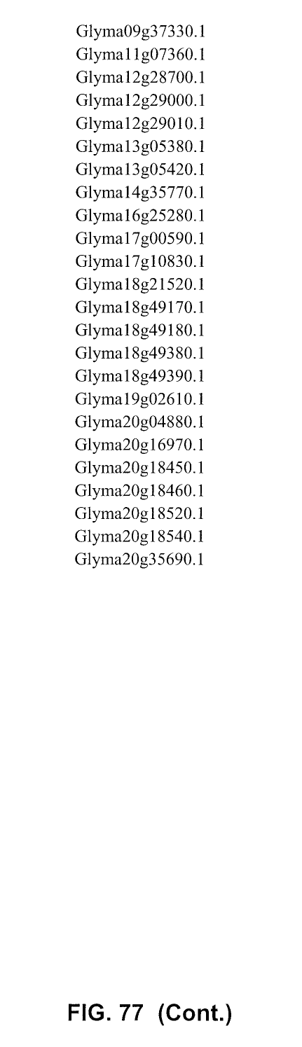

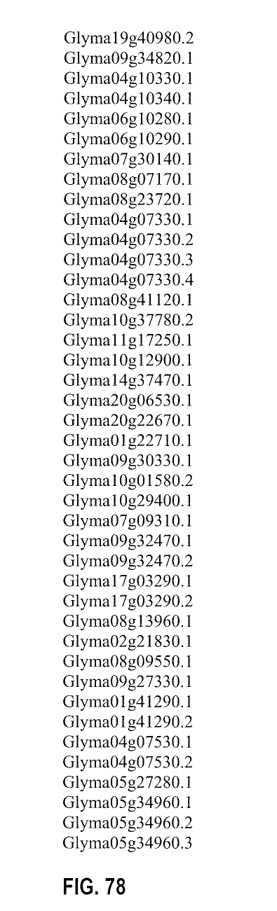

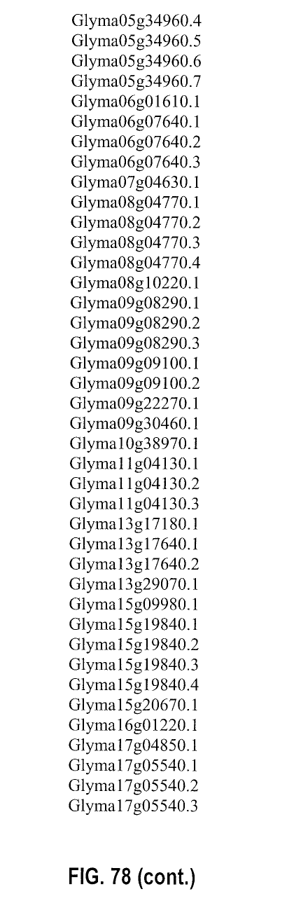

Soybean transcription factors belonging to different families are shown in FIG. 1. The Soybean Database Identification numbers of members of these families are shown in FIGS. 15-78. The sequences of the genes coding for these proteins and the proteins themselves may be obtained from the Soybean Genome Databases maintained by the University of Missouri at Columbia which may be accessed freely by the general public. The links for some of these databases are listed below:

http://casp.rnet.missouri.edu/soydb
http://www.phytozome.net/soybean.php and
http://www.phytozome.net/cgi-bin/gbrowse/soybean/?start=5935000; stop=6024999; ref=Gm01; width=800; version=100;
cache=on; drag and drop=on; show_tooltips=on; grid=on; label=Transcripts-Glycine_max_est-Gmax_PASA_assembly

The sequences of all genes or proteins listed in this disclosure or those referenced by PublicID, GenBank ID, or soybean gene ID are hereby incorporated by reference into this disclosure as if fully reproduced herein.

### Example 11

### Bioinformatic Analysis of Soybean Transcription Factors to Identify the Enrichment or Depletion of Specific Transcription Factor Families in Soybean when Compared to Other Model Plant Species

The amino acid sequences of the TFs in each 64 Arabidopsis TF families were downloaded from DATF (Guo, et al., 2005) and the sequences were aligned by a multiple sequence alignment tool MUSCLE (Edgar, 2004). A hidden Markov model was trained for each Arabidopsis family by SAM (Hughey and Krogh, 1995) using the multiple sequence alignment. Each of the 6,690 soybean TFs was aligned individually to each of the 64 hidden Markov models and then was assigned to the TF family whose hidden Markov model generated the lowest e-value. This e-value indicates the fitness between the query TF sequence and the hidden Markov model, with smaller e-value indicating better fitness between them. Out of the entire soybean TFs, the highest e-value was 0.305 on one soybean TF, and a total of 166 soybean TFs had an e-value between 0.1-0.4, which indicates most of the soybean TFs had a confident classification to one of the 64 TF families from Arabidopsis.

Comparisons of TF numbers in each TF family between soybean and Arabidopsis: The numbers of transcription factors in each of the 64 families for soybean and Arabidopsis were compared (Table 1). For each family, the TF number of soybean was divided by the one in Arabidopsis. A higher ratio shows the families have an enriched number of soybean transcriptions as compared to Arabidopsis. Based on TAIR version 8 (Rhee, et al., 2003), Arabidopsis has 32,825 proteins, while soybean has 75,778 proteins based on the soybean genome sequencing completed in early 2008 by the Department of Energy-Joint Genome Institute (Schmutz, et al., 2009). Therefore, the soybean gene number is about two times bigger than Arabidopsis, and the >2.3 ratio (75,778/32,825) in Table 1 shows enrichment in soybean after considering the genome size difference between these two species.

TABLE 8
The comparisons of number of transcription factors (gene models)
in every soybean and Arabidopsis TF family, ranked by the
ratio of soybean sequence number divided by the Arabidopsis
sequence number.

| | Soybean | Arabidopsis | |
|---|---|---|---|
| Family Name | Num. | Num. | Ratio |
| GeBP | 12 | 21 | 0.6 |
| BBR-BPC | 12 | 13 | 0.9 |
| HSF | 30 | 24 | 1.2 |
| PcG | 51 | 44 | 1.2 |
| GRF | 14 | 9 | 1.6 |
| NIN-like | 28 | 16 | 1.8 |
| NAC | 221 | 117 | 1.9 |
| S1Fa-like | 6 | 3 | 2 |
| bZIP | 237 | 107 | 2.2 |
| AS2 | 100 | 45 | 2.2 |
| CCAAT-DR1 | 12 | 5 | 2.4 |
| MADS | 279 | 118 | 2.4 |
| C2C2-DOF | 105 | 43 | 2.4 |
| SRS | 31 | 13 | 2.4 |
| CCAAT-HAP5 | 47 | 19 | 2.5 |
| CCAAT-HAP3 | 45 | 18 | 2.5 |
| E2F-DP | 37 | 15 | 2.5 |
| C2H2 | 372 | 145 | 2.6 |
| BES1 | 34 | 13 | 2.6 |
| AP2-EREBP | 425 | 159 | 2.7 |
| ZIM | 76 | 27 | 2.8 |
| GARP-G2-like | 157 | 56 | 2.8 |
| TCP | 75 | 27 | 2.8 |
| Trihelix | 80 | 29 | 2.8 |
| LUG | 20 | 7 | 2.9 |
| bHLH | 487 | 158 | 3.1 |
| C2C2-CO-like | 142 | 46 | 3.1 |
| AUX-IAA | 105 | 34 | 3.1 |
| C3H | 211 | 69 | 3.1 |
| HB | 304 | 98 | 3.1 |
| MYB-related | 211 | 65 | 3.2 |
| CPP | 29 | 9 | 3.2 |
| PHD | 215 | 65 | 3.3 |
| Alfin | 31 | 9 | 3.4 |
| SBP | 91 | 27 | 3.4 |
| C2C2-GATA | 104 | 30 | 3.5 |
| MYB | 574 | 165 | 3.5 |
| ZD-HD | 59 | 17 | 3.5 |
| ARF | 129 | 34 | 3.8 |
| TLP | 62 | 16 | 3.9 |
| EIL | 24 | 6 | 4 |
| HMG | 75 | 17 | 4.4 |
| ULT | 9 | 2 | 4.5 |
| CCAAT-HAP2 | 23 | 5 | 4.6 |
| MBF1 | 14 | 3 | 4.7 |
| GRAS | 164 | 35 | 4.7 |
| GARP-ARR-B | 53 | 11 | 4.8 |
| LIM | 86 | 18 | 4.8 |
| FHA | 93 | 17 | 5.5 |
| PLATZ | 60 | 11 | 5.5 |
| JUMONJI | 112 | 20 | 5.6 |
| ARID | 64 | 11 | 5.8 |
| CAMTA | 41 | 7 | 5.9 |
| GIF | 18 | 3 | 6 |
| HRT-like | 12 | 2 | 6 |
| ABI3-VP1 | 101 | 16 | 6.3 |
| C2C2-YABBY | 43 | 6 | 7.2 |
| TAZ | 76 | 10 | 7.6 |
| WRKY | 245 | 30 | 8.2 |
| SAP | 10 | 1 | 10 |
| Whirly | 21 | 2 | 10.5 |
| VOZ | 34 | 2 | 17 |
| NZZ | 18 | 1 | 18 |
| LFY | 34 | 1 | 34 |

The functions of the top 5 and bottom 5 TF families ranked by the TF number ratio between soybean and Arabidopsis are listed in Table 9. The functions are cited from the database DATF (Guo, et al., 2005). As shown in Table 9, soybean TFs are mostly enriched in those families that are involved in reproductions, such as pollen and flower development.

TABLE 9
The brief functions of the top and bottom 5 families ranked
by the ratio of soybean TF number divided by Arabidopsis
TF number.

| Family | ratio | |
|---|---|---|
| GeBP | 0.6 | GL1 enhancer binding protein, acting as a repressor of |
| | | leaf cell fate |
| BBR-BPC | 0.9 | Regulate gene SEEDSTICK (STK), which controls |
| | | ovule identify, and characterized its mechanism of |
| | | action |
| HSF | 1.2 | Heat shock transcription factor, responsible for |
| | | relaying signals of cellular stress to the transcriptional |
| | | apparatus |
| PcG | 1.2 | PcG mutants exhibit posterior transformations in |
| | | embryos and adults caused by depression of homeltic |
| | | loci in flies, and in vertebrates, also regulate non- |
| | | homeotic targets. |
| GRF | 1.6 | Plays a regulatory role in stem elongation |
| SAP | 10 | Involved in the initiation of female gametophyte |
| | | development |
| Whirly | 10.5 | Activate pathogenesis-related genes |
| VOZ | 17 | Control V-PPase for pollen development |
| NZZ | 18 | Develop and control sporangia |
| LFY | 34 | Controls the production of flowers |

### Example 12

### Tissue Specific and Nodulation Related Expression Pattern of Soybean Transcription Factors

qRT-PCR provides one of the most accurate methods to quantify gene expression. Using this technology, the expression of 1034 out of the 5671 transcription factor genes (TF) identified in soybean (18%) was quantified during soybean root nodulation and in different tissues. See Example 2. The entire soybean genome has been published. See e.g., Schmutz et al., 2010. To better understand the regulation of soybean TF gene expression, it is important to note that two duplication events occurred in the soybean genome about 59 and 13 million years ago, respectively. These duplications have led to multiple copies of the same gene in the soybean genome which is also called homeologous genes.

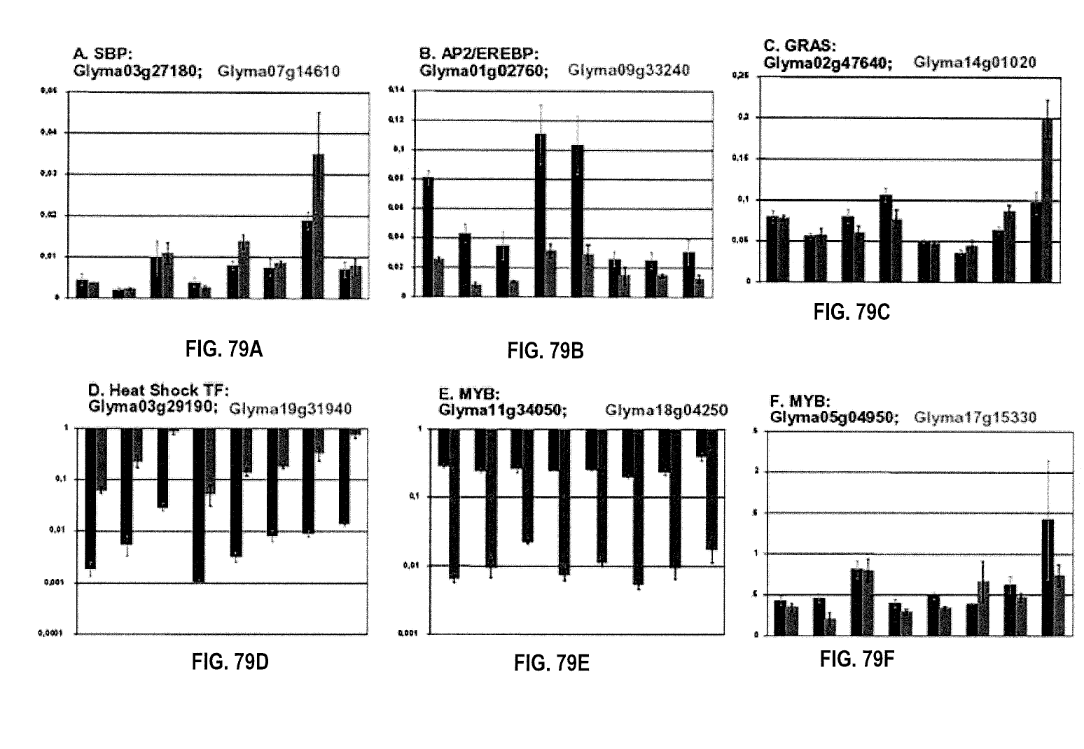

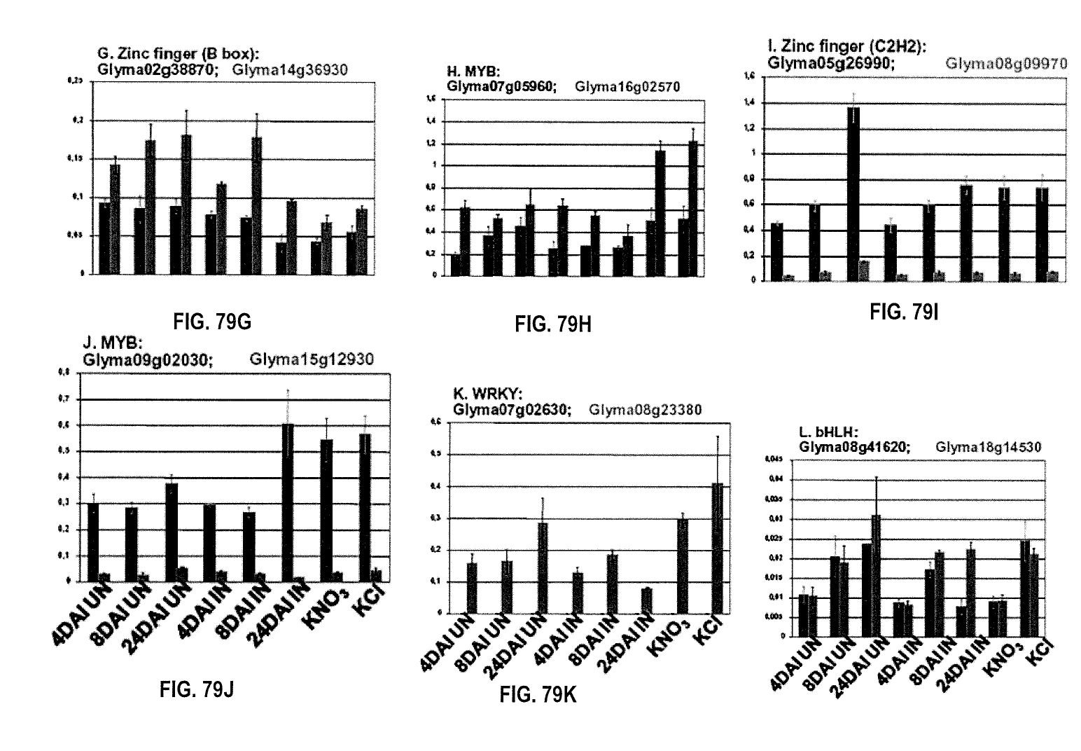

The expression levels of homeologous soybean genes during soybean root nodulation and in response to KCl and KNO₃ were compared using the qRT-PCR data (FIG. 79). The expression of homeologs quantified by qRT-PCR can diverge significantly after duplication of soybean genome. On each graphic, the expression of the two homeologs is indicated in grey and black. Transcription factor transcripts from 4, 8 and 24 days after inoculation (DAI) roots inoculated (IN) or mock-inoculated (UN) with B. japonicum and roots treated with KCl and KNO3 (x-axis) were normalized against the soybean reference gene Cons6 (y-axis).

This analysis unveiled numerous examples of homeologous soybean TF genes showing differential expression (FIG. 79) and the complete extinction of the expression of one of the duplicated genes (FIG. 79-K). Such gene is also called pseudogene.

Despite the value of such analysis, it was frustrating to limit our analysis to a small fraction of the soybean TF genes. The restricted number of soybean TF genes analyzed by qRT-PCR is mainly limited by the design of specific primers for each gene analyzed. Consequently, the use of technologies such as Illumina-Solexa technology allowing the accurate quantification of the transcriptome of the entire set of soybean TF genes is required. Illumina-Solexa technology allows quantifying very accurately the expression of transcripts including low abundant transcripts such as TF gene transcripts and is not restricted to a subset of the soybean genes

Despite the value of such analysis, the number of soybean TF genes that can be analyzed by qRT-PCR is limited by the design and synthesis of specific primers for each gene analyzed. The use of technologies such as Illumina-Solexa technology may allow the accurate quantification of the transcriptome of the entire set of soybean TF genes. Illumina-Solexa technology may enable very accurate quantification of the expression of genes including low-abundance transcripts such as TF gene transcripts and is not restricted to a subset of the soybean genes.

With the help of the Illumina-Solexa technology, a soybean transcriptome atlas has been developed which shows, among others, the expression of the 5671 soybean TF genes across 14 different conditions and/or location, namely, Bradyrhizobium japonicum-inoculated and mock-inoculated root hairs isolated 12, 24 and 48 hours after inoculation, Bradyrhizobium japonicum-inoculated stripped root isolated 48 hours after inoculation (i.e. root devoid of root hair cells), mature nodule, root, root tip, shoot apical meristem, leaf, flower, green pod (Table 10). The upper half of Table 10 shows expression of these genes in 7 conditions/tissues, while the lower half of Table 10 shows expression of the same genes in the remaining 7 conditions/tissues. No transcripts were detected across the 14 conditions tested for 787 soybean TF genes (Table 10). Although this set of conditions is not exhaustive; this result suggests that these 787 genes might be pseudogenes (i.e. genes silenced during their evolution). Such a result confirmed previous reports based on qRT-PCR as described above.

This large scale analysis also enables the identification of soybean TF genes showing a repetitive induction of their expression during root hair cell infection by B. japonicum (Table 11). It is worth noting that some of these soybean TF genes were orthologs to Lotus japonicus and Pisum sativum TF genes that have been previously identified as key-regulators of the root hair infection by rhizobia (Table 11).

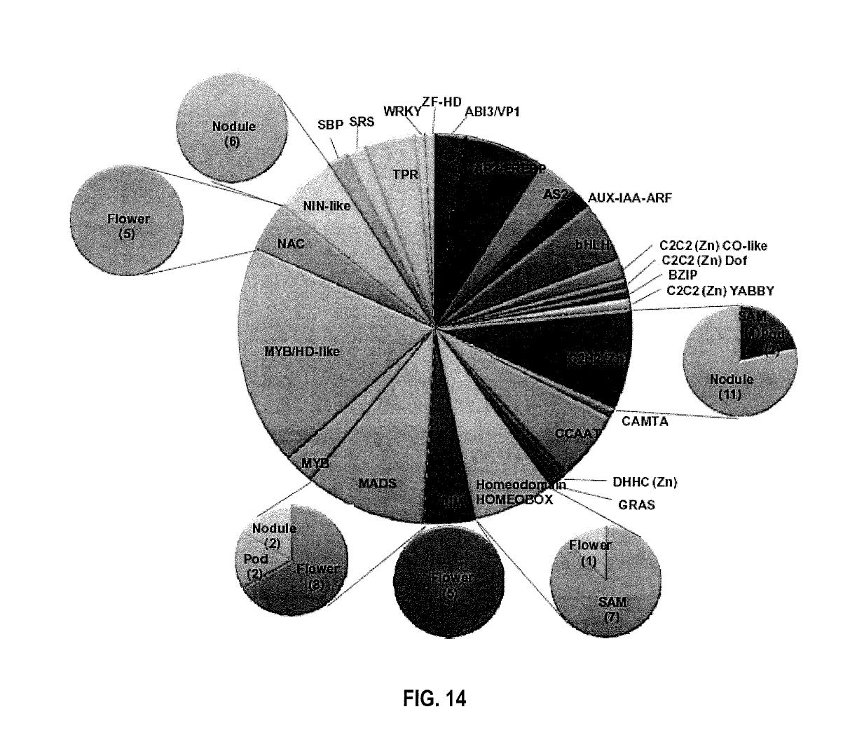

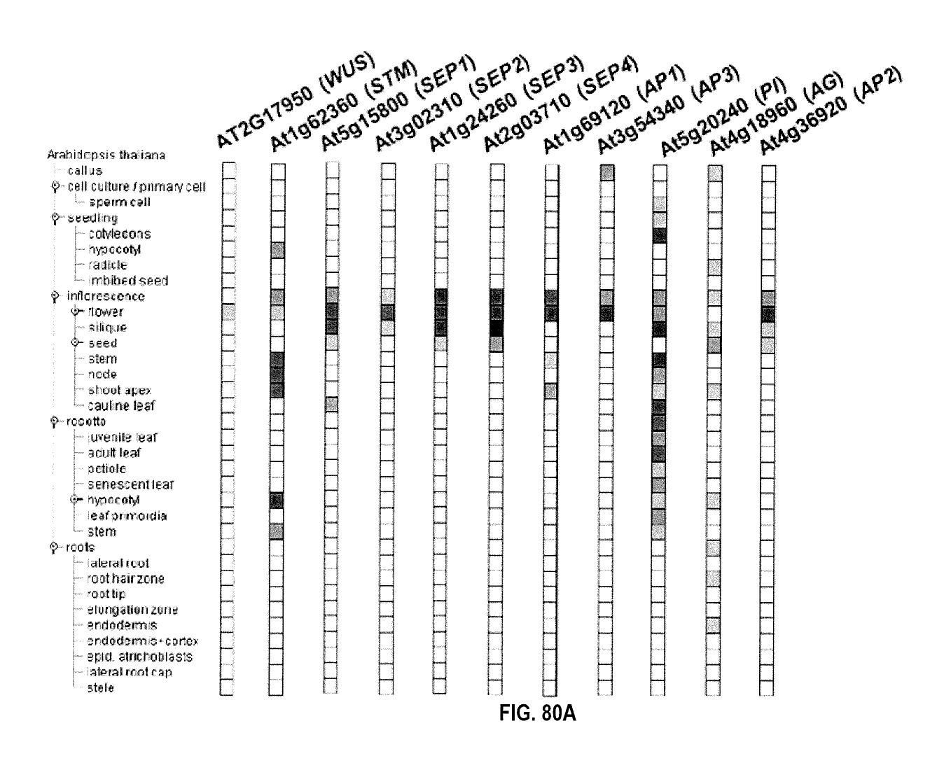

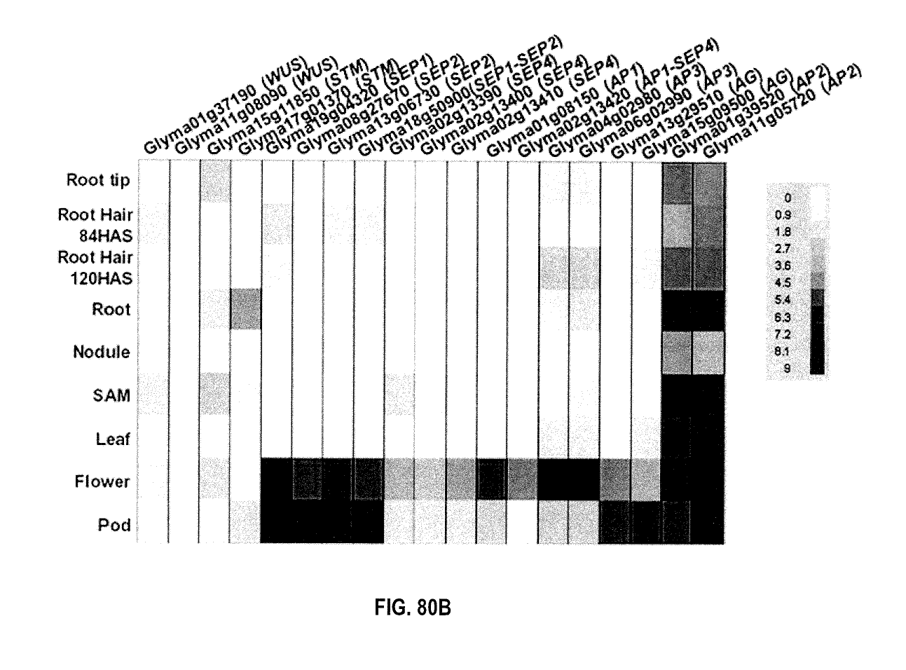

120 soybean TF genes were identified which were expressed at least 10 times more in one soybean tissues when compared to the remaining 9 tissues (i.e. mock-inoculated root hairs isolated 12 and 48 hours after treatment, mature nodule, root, root tip, shoot apical meristem, leaf, flower, green pod. See FIG. 14 and Table 12. By comparing our list to previously published data, we were able to identify the soybean orthologs of Arabidopsis proteins regulating floral development (FIG. 80). Taken together, these analyses confirm the relatively high quality of the soybean TF gene expression profiles as quantified by Illumina-Solexa technology.

Lengthy table referenced here
US20120198587A1-20120802-T00001
Please refer to the end of the specification for access instructions.

Lengthy table referenced here
US20120198587A1-20120802-T00002
Please refer to the end of the specification for access instructions.

Lengthy table referenced here
US20120198587A1-20120802-T00003
Please refer to the end of the specification for access instructions.

Lengthy table referenced here
US20120198587A1-20120802-T00004
Please refer to the end of the specification for access instructions.

### Example 13

### Expression Pattern of Members of Nac Family of Transcription Factors (TFs) and Analysis of the Transgenic Arabidopsis Plants Harboring the Same

NAC transcription factors (TFs) are plant specific transcription factors that have been reported to enhance stress tolerance in number of plant species. The NAC TFs regulate a number of biochemical processes which protect the plants under water-deficit conditions. A comprehensive study of the NAC TF family in Arabidopsis reported that there are 105 putative NAC TFs in this model plant. More than 140 putative NAC or NAC-like TFs have been identified in Rice. The NAC TFs are multi-functional proteins and are involved in a wide range of processes such as abiotic and biotic stress responses, lateral root and plant development, flowering, secondary wall thickening, anther dehiscence, senescence and seed quality, among others.

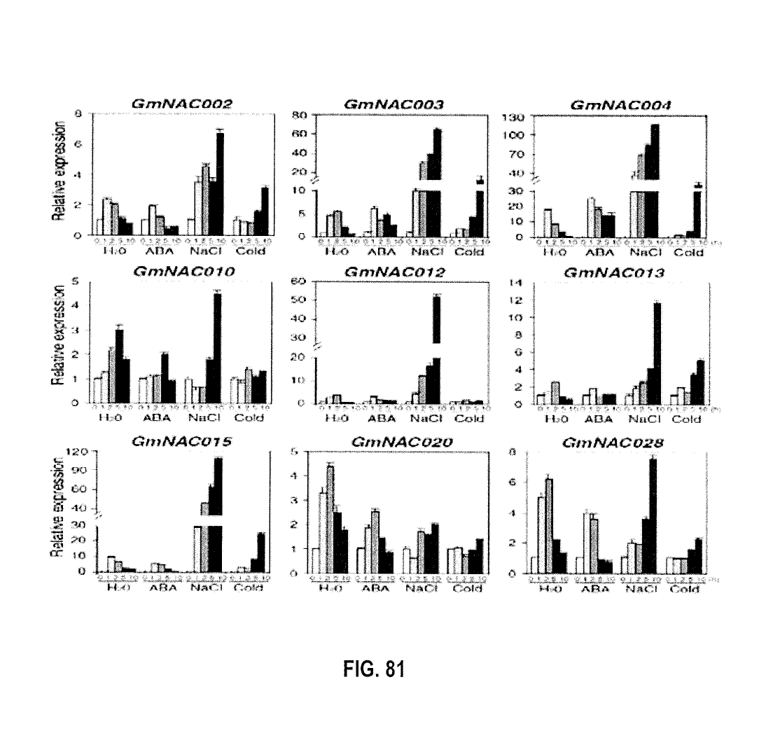

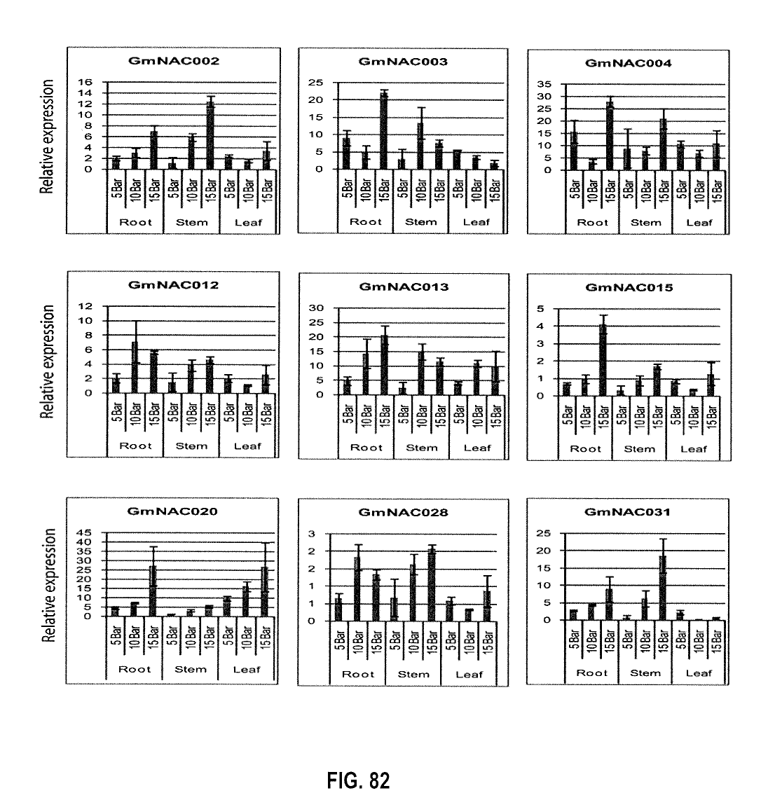

170 potential NACs were identified through the soybean genome sequence analysis. Full length sequence information of 41 GmNACs are available at present and 31 of them are cloned. Quantitative real time PCR experiments were conducted to identify tissue specific and stress specific NAC transcription factors in soybean and the results are shown in FIGS. 81 and 82. Briefly, soybean seedling tissues were exposed to dehydration, abscisic acid (ABA), sodium chloride (NaCl) and cold stresses for 0, 1, 2, 5 and 10 hours and the total RNAs were extracted for this study. The cDNAs were generated from the total RNAs and the gene expression studies were conducted using ABI 7990HT sequence detection system and delta delta Ct method.

The drought response of these genes was studied, and the results are shown in FIG. 84. Briefly, drought stress was imposed by withholding water and the root, leaf and stem tissues were collected after the tissue water potential reaches 5 bar, 10 bar and 15 bar (representing various levels of water stress). Total RNAs were extracted from these tissues and the gene expression studies were conducted using the ABI 7900 HT sequence detection system. These experiments revealed tissue specific and stress specific NAC TFs and the expression pattern of these specific NAC family members.

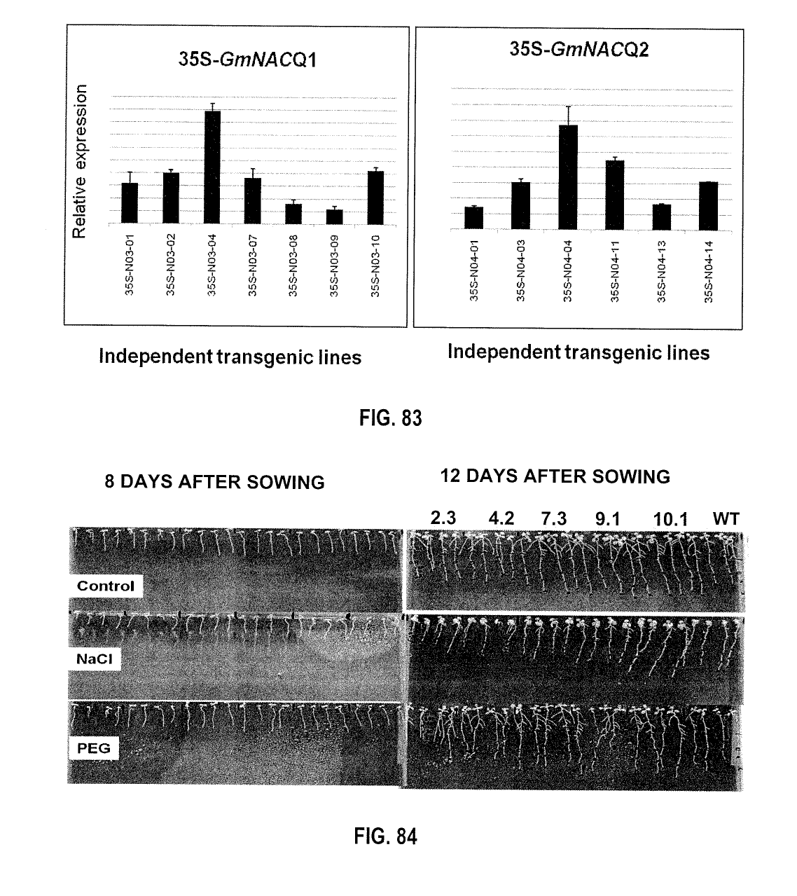

A number of NAC TFs were cloned and expressed in the Arabidopsis plants to study the biological functions in-planta. Transgenic Arabidopsis plants were developed and assayed for various physiological, developmental and stress related characteristics. Two of the major gene constructs (following gene cassettes) were utilized for the transgene expression in Arabidopsis plants. One is CaMV35S Promoter-GmNAC3gene-NOS terminator, the other construct is CaMV35S Promoter-GmNAC4gene-NOS terminator. The coding sequence of the GmNAC3 gene is listed as SEQ ID No. 2299, while the coding sequence of the GmNAC4 gene is listed as SEQ ID No. 2300. For the transgenic experiments, the Arabidopsis ecotype Columbia was transformed with the above gene constructs using floral dip method and the transgenic plants were developed. Independent transgenic plants were assayed for the transgene expression levels using qRT-PCR methods (FIG. 83). (Q1 is the independent transgenic lines expressing GmNAC3 and Q2 is the independent transgenic lines expressing GmNAC4).

Examination of the transgenic plants revealed that the transgenic plants showed improved root growth and branching as compared to controls (FIG. 84). Because the root system plays an important role in drought response, these transgenic plants have the potential for drought tolerance. These DRG candidates and the constructs may be used to produce transgenic soybean plants expressing these genes. The DRG candidate genes may also be placed under control of a tissue specific promoter or a promoter that is only turned on during certain developmental stages. For instance, a promoter that is on during the growth phase of the soybean plant, but not during later stage when seeds are being formed.

A trend towards the enhanced root branching (more lateral roots) was observed under simulated drought stress conditions using the poly ethylene glycol (PEG) containing growth medium. Major observations during these studies include, for example, GmNACC3 and GmNACC4 are differentially expressed in soybean root, and both seemed to be expressed at a higher level in the root. It is likely that the proteins encoded by the transgenes in GmNACQ1 and GmNACQ2 help regulate lateral root development in transgenic Arabidopsis plants.

### Example 14

### Transgenic Arabidopsis Plants with GmC2H2 Transcription Factor and GmDOF27 Transcription Factor Shows Better Plant Growth and Development Characteristics

To identify other proteins that may be beneficial to a host plant, Arabidopsis transgenic plants with the following gene constructs were generated: (a) CaMV35S Promoter-GmC2H2 gene-NOS terminator; and (b) CaMV35S Promoter-GmDOF27 gene-NOS terminator. The coding sequence of the GmC2H2 gene is listed as SEQ ID No. 2301, while the coding sequence of the GmDOF27 gene is listed as SEQ ID No. 2302. The homozygous transgenic lines (T3 generation) were developed and the physiological assays were conducted, including, for example, examination of root and shoot growth, stress tolerance, and yield characteristics.

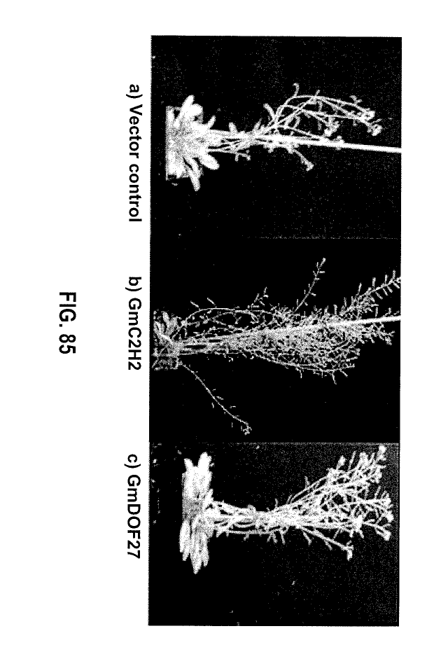

FIG. 85 shows comparison of the vector control and transgenic plants morphology at the reproductive stage. There appeared to be distinct differences between the control and transgenic Arabidopsis plants in shoot growth and flowering and silique intensity. Further analysis is conducted to examine the biomass changes, root growth and seed yield characteristics under well watered and water stressed conditions.

While the foregoing instrumentalities have been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

### REFERENCES

In addition to those references that are cited in full in the text, additional information for those abbreviated citations is listed below:
Boyer, J S, 1983, Environmental stress and crop yields. In C. D. Raper and P. J. Kramer (ed) Crop reactions to water and temperature stresses In humid, temperature climates. Westview press, Boulder, Colo. pp 3-7.
Muchow R C, Sinclair T R. 1988. Water and nitrogen limitations In soybean grain production. II. Field and model analyses. Field Crop Res. 15:143-158.
Specht J E, Hume D J, Kumind S V. 1999. Soybean yield potential-A genetic physiological perspective. Crop Science 39:1560-1570.
Wang W, Vinocur B, Altman A: Plant responses to drought, salinity and extreme temperatures: towards genetic engineering for stress tolerance. Planta 2003, 218:1-14.
Vinocur, B, Altman A: Recent advances in engineering plant tolerance to abiotic stress: achievements and limitations. Curr Opin Biotech 2005, 16:123-32.
Chaves M M, Oliveire M M: Mechanisms underlying plant resilience to water deficits: prospects for water-saving agriculture. J Exp Bot 2004, 55; 2365-2384.
Shinozaki K, Yamaguchi-Shinozaki K, Seki M: Regulatory network of gene expression in the drought and cold stress responses. Curr Opin Plant Biol 2003, 6:410-417.
Schena M, Shalon D, Davis R W, Brown PO (1995) Quantitative monitoring of gene expression patterns with a complementary DNA microarray. Science 270: 467-470
Shalon D, Smith S, Brown P (1990) A DNA microarray system for analyzing complsx DNA samples using two-color fluorescent probe hybridization. Genome Res. 8: 639-645.
Bray E A: Genes commonly regulated by water-deficit stress in Arabidopsis thaliana. J Exp Bot 2004, 55:2331-2341.
Denby K, Gehring C: Engineering drought and salinity tolerance in plants: lessons from genome-wide expression profiling In Arabidopsis. Trends in Plant Sci 2005, 23547-552.
Shinozaki K, Yamaguchi-Shinozaki K: Molecular responses to drought and cold stress. Curr Opin Biotech 1996, 7:181-167
Shinozaki. K, and Yamaguchi-Shinozaki, K: Molecular responses to dehydration and low temperature; differences and cross-talk between two stress signaling pathways. Curr Opin Plant Biol 2000, 3:217-223.
Seki M, Narusaka M, Abe H, Kasuga M, Yamaguchi-Shinozaki K, Carninci P, Hayashizaki Y, Shinozaki K: Monitoring the expression pattern of 1300 Arabidopsis genes under drought and cold stresses by using a full-length cDNA microarray. Plant Cell 2001, 13:61-72.
Fowler S, Thomashow M F: Arabidopsis transcriptome profiling indicates that multiple regulatory pathways are activated during cold acclimation In addition to the CBF cold response pathway, Plant Cell 2002, 14:1875-1690.
Maruyama K, Sakuma Y, Kasuga M, Ito Y, Seki M, Goda H, Shimada Y, Yoshida S, Shinozaki K, Yamaguchi-Shinozaki K: identification of cold-inducible downstream genes of the Arabidopsis DREB1A/CBF3 transcriptional factor using two microarray systems. Plant J 2004, 38:982-993.
Edgar, R. (2004) MUSCLE: multiple sequence alignment with high accuracy and high throughput, Nucleic Acids Research, 32, 1792-1797.
Guo, A., He, K., Liu, D., Bai, S., Gu, X., Wei, L. and Luo, J. (2005) DATF: a database of Arabidopsis transcription factors, Bioinformatics, 21, 2568-2569.
Hughey, R. and Krogh, A. (1995) SAM: sequence alignment and modeling software system. In, Technical Report: UCSC—CRL-95-07. University of California at Santa Cruz.
Rhee, S., Beavis, W., Berardini, T., Chen, G., Dixon, D., Doyle, A., Garcia-Hernandez, M., Huala, E., Lander, G., Montoya, M., Miller, N., Mueller, L., Mundodi, S., Reiser, L., Tacklind, J. and Weems, D. (2003) The Arabidopsis Information Resource (TAIR): a model organism database providing a centralized, curated gateway to Arabidopsis biology, research materials and community, Nucleic Acids Research, 224-228.
Schmutz, J., Cannon, S., Schlueter, J et al. (2010) Genome sequence of the paleopolyploid soybean (Glycine max (L.) Merr.). Nature, 463 (7278):178-183.

LENGTHY TABLES
The patent application contains a lengthy table section. A copy of the table is available in electronic form from the USPTO web site (http://seqdata.uspto.gov/?pageRequest=docDetail&DocID=US20120198587A1). An electronic copy of the table will also be available from the USPTO upon request and payment of the fee set forth in 37 CFR 1.19(b)(3).

We claim:

1. A method for generating a transgenic plant from a host plant, said transgenic plant being more tolerant to an adverse condition when compared to the host plant, said method comprising a step of altering the expression levels of a transcription factor or fragment thereof, said adverse condition being at least one condition where one or more of an environmental conditions is too high or too low, said environmental condition being selected from a group consisting of water, salt, acidity, temperature and combination thereof, the expression of said transcription factor being upregulated or downregulated in an organism in response to said adverse condition.

2. The method of claim 1, wherein said organism is a second plant that is different from said host plant.

3. The method of claim 1, wherein said transcription factor is exogenous to said host plant.

4. The method of claim 1, wherein said transcription factor is derived from a plant that is genetically different from the host plant.

5. The method of claim 4, wherein said transcription factor is derived from a plant belonging to the same species as the host plant.

6. The method of claim 1, wherein the transcription factor is encoded by a coding sequence selected from the group consisting of the polynucleotide sequence of SEQ ID. No. 2299, SEQ ID. No. 2300, SEQ ID. No. 2301, and SEQ ID. No. 2302.

7. The method of claim 1, wherein the coding sequence of said transcription factor or a fragment thereof is operably linked to a promoter for regulating expression of said polypeptide.

8. The method of claim 7, wherein the promoter is derived from another gene that is different from the gene encoding said transcription factor.

9. The method of claim 2, wherein the expression of said transcription factor is upregulated or downregulated in said second plant in response to said adverse condition by at least a two-fold changes in expression levels.

10. A method for generating a transgenic plant from a host plant, said transgenic plant being more tolerant to an adverse condition when compared to the host plant, said method comprising the steps of: (a) introducing into a plant cell a construct comprising a regulatory sequence and a coding sequence encoding a first polypeptide, said regulatory sequence being at least 90% identical to the promoter sequence of a second polypeptide, wherein the second polypeptide is a transcription factor, the expression of said transcription factor being upregulated or downregulated in an organism in response to said adverse condition, said adverse condition being at least one condition where one or more of an environmental condition is too high or too low, said environmental condition being selected from a group consisting of water, salt, acidity, temperature and combination thereof, and (b) generating a transgenic plant expressing said first polypeptide.

11. The method of claim 10, wherein the coding sequence is operably linked to the regulatory sequence whereby the expression of the first polypeptide is regulated by the regulatory sequence.

12. The method of claim 10, wherein said organism is a second plant that is different from said host plant.

13. The method of claim 10, wherein the regulatory sequence is a promoter that is at least one member selected from the group consisting of a cell-specific promoter, a tissue specific promoter, an organ specific promoter, a constitutive promoter, and an inducible promoter.

14. The method according to claim 13, wherein at least a portion of said coding sequence is oriented in an antisense direction relative to said promoter within said construct.

15. The method of claim 10, wherein the adverse condition is drought.

16. A transgenic plant generated from a host plant using the method of claim 1, or claim 10, said transgenic plant exhibiting increased tolerance to the adverse condition as compared to the host plant.

17. The transgenic plant of claim 16, wherein the transcription factor is encoded by a coding sequence selected from the group consisting of the polynucleotide sequence of SEQ ID. No. 2299, SEQ ID. No. 2300, SEQ ID. No. 2301, and SEQ ID. No.

18. The transgenic plant of claim 17, wherein the coding region of the transcription factor is operably linked to a promoter for regulating expression of said transcription factor.

19. The transgenic plant of claim 18, wherein the promoter is at least one member selected from the group consisting of a cell-specific promoter, a tissue specific promoter, an organ specific promoter, a constitutive promoter, and an inducible promoter.

20. The transgenic plant of claim 16, wherein the host plant is selected from the group consisting of soybean, corn, wheat, rice, cotton, sugar cane, and Arabidopsis.
